(19) United States
(12) Patent Application Publication
(10) Pub. No.: US 2024/0408222 A1
(43) Pub. Date: Dec. 12, 2024

(54) COMPOUNDS, PHARMACEUTICAL COMPOSITIONS, AND METHODS FOR THE TREATMENT, PREVENTION, OR MANAGEMENT OF HYPERPROLIFERATIVE DISORDER

(71) Applicants: VINCERX PHARMA GMBH, Monheim am Rhein (DE); VINCERX PHARMA, INC., Palo Alto, CA (US)

(72) Inventors: Hans-Georg LERCHEN, Leverkusen (DE); Beatrix STELTE-LUDWIG, Wülfrath (DE); Mareike WIEDMANN, Monheim am Rhein (DE); Andreas LERCHEN, Münster (DE); Anne-Sophie REBSTOCK, Champagne au Mont d'Or (FR); Johannes KOEBBERLING, Neuss (DE); Harvey WONG, Vancouver, BC (CA)

(21) Appl. No.: 18/698,333

(22) PCT Filed: Oct. 3, 2022
(86) PCT No.: PCT/IB2022/000556

§ 371 Date: Apr. 3, 2024

Related U.S. Application Data
(60) Provisional application No. 63/252,116, filed on Oct. 4, 2021.

(51) Int. Cl. A61K 47/65; A61K 31/444; A61K 31/506; A61K 31/53; A61P 35/00

(52) U.S. Cl. CPC A61K 47/65; A61K 31/444; A61K 31/506; A61K 31/53; A61P 35/00

(57) ABSTRACT

Disclosed herein are compounds, pharmaceutical compositions, and methods for treating, preventing or managing diseases and conditions including hyperproliferative disorders such as cancer in humans and other mammals. Compounds disclosed herein are PTEFb inhibitor prodrugs, conjugated to an integrin binding moiety via cleavable linkers and/or functional spacers.

### CROSS-REFERENCE

This International Patent Application claims the benefit of U.S. Provisional Patent Application No. 63/252,116, filed Oct. 4, 2021, which is incorporated herein by reference in its entirety.

### BACKGROUND OF THE INVENTION

This application relates to compounds, pharmaceutical compositions, processes for preparation thereof, and the use thereof for treating, preventing, or managing diseases and conditions including hyperproliferative disorders, such as, cancer in humans and other mammals.

### SUMMARY OF THE INVENTION

The invention relates to novel compounds (or “conjugates”) comprising one or more binder molecules or derivatives thereof with one or more molecules of an active component, wherein the active component is a CDK9 kinase inhibitor (e.g., a P-TEFb inhibitor), which is conjugated to one more binder molecules (e.g., an integrin binder (e.g., an αᵥβ₃ integrin binder)) via a linker peptide EL as described and defined herein, and methods for their preparation, their use for the treatment and/or prophylaxis of disorders, in particular of hyper-proliferative disorders.

In WO 2017/060322, small molecule drug conjugates have been described with binding peptides or proteins, which are internalized upon binding. Small molecule drug conjugates may show favorable features over ADCs such as e. g. higher tumor penetration (Cazzamalli et al., J. Am. Chem. Soc. 2018, 140, 1617).

The current invention describes conjugates with small molecule binders, which release the drug extracellularly in the tumor microenvironment (TME) upon cleavage by enzymes present in TME. Compounds described herein may comprise small molecule binders (e.g., an integrin binder), conjugated via an enzymatically cleavable linker and/or a polymeric linker to a CDK9 inhibitor, which, in some embodiments, is retained in an acidic TME.

To increase the therapeutic window of CDK9 inhibitors and to achieve a tumor targeting of this efficacious class of anti-tumor compounds, a novel class of CDK9 inhibitor prodrugs has been developed and this is described in the current invention. The inventive prodrug compounds may comprise, but are not limited to, one or more αᵥβ₃ integrin binding moieties which are linked to a CDK9 inhibitor via a peptide moiety EL, which is cleaved by proteases present in the tumor microenvironment (TME) to release the parent CDK9 inhibitor compound at the site of action. Enzymes present in the TME include, but are not limited to, neutrophil elastase, cathepsins such as cathepsin B and legumain.

In a particular embodiment, the current invention describes conjugates where the peptide moiety (EL) is linked to a sulfoximine moiety present in the CDK9 inhibitor. It was found that conjugates of the current invention containing substrate peptides of tumor stromal enzymes, such as neutrophil elastase in position P1-P3 and the sulfoximine moiety of the CDK9 inhibitor molecule in P1′ are particularly stable in plasma, however, are cleaved surprisingly well by these enzymes to release the CDK9 inhibitor at its site of action. While the αvβ3-linker-CDK9 conjugates showed only weak cytotoxic activity in the absence of the respective cleavage enzyme, the cytotoxic activity was significantly increased in the presence of tumor-associated enzymes such as neutrophil elastase.

In one aspect, provided herein is a compound, or a pharmaceutically acceptable salt thereof, comprising a CDK-9 (e.g., PTEFb) inhibitor conjugated to one or more integrin binders via a linker. In some embodiments, the linker is enzymatically cleavable.

In some embodiments, provided herein is a compound, or a pharmaceutically acceptable salt thereof, according to the formula:

IN-L-EL-PT
wherein:
PT is a PTEFb inhibitor;
EL is a peptide linker, optionally further comprising a self-immolative group;
L is a linker, optionally further comprising a second integrin binder; and
IN is an integrin binder.

In some embodiments, the compound is of the formula:

IN-L-AA¹-AA²-(AA³)₀₋₁-(SIL)₀₋₁-PT
wherein:
PT is a PTEFb inhibitor;
SIL is a self-immolative linker;
AA¹ is an amino acid;
AA² is an amino acid;
AA³ is an amino acid;
L is a linker, optionally further comprising a second integrin binder; and
IN is an integrin binder.

In some embodiments, the compound has the structure:

IN-L-AA¹-AA²-AA³SIL-PT,

IN-L-AA¹-AA²-AA³-PT,

IN-L-AA¹-AA²-SIL-PT, or

IN-L-AA¹-AA²-PT;
wherein:
PT is a PTEFb inhibitor;
SIL is a self-immolative linker;
AA¹ is an amino acid;
AA² is an amino acid;
AA³ is an amino acid;
L is a linker, optionally further comprising a second integrin binder; and
IN is an integrin binder.

In some embodiments, the compound has the structure:

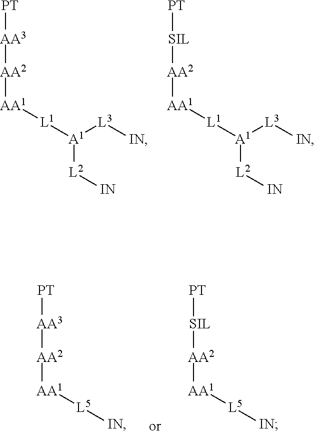

wherein:
PT is a PTEFb inhibitor;
SIL is a self-immolative linker (e.g., a PABC linker);
each AA¹, AA², and AA³ is independently an amino acid,
each L¹, L², L³, and L⁵ is independently a bivalent linker,
A¹ is a trivalent linker; and
IN is an integrin binder.

In some embodiments, IN is a peptidic or peptidomimetic integrin binder. In some embodiments, IN is small molecule integrin binder. In some embodiments, IN is a small molecule αᵥβ₃ integrin binder. In some embodiments, EL is enzymatically-cleavable. In some embodiments, EL is cleavable by cathepsin B, legumain, or neutrophil elastase. In some embodiments, the group -AA¹-AA²-(AA³)₀₋₁-(SIL)₀₋₁- is cleaved by cathepsin B, legumain, or neutrophil elastase, wherein each AA is an amino acid and SIL is an optional self-immolative linker.

In some embodiments, the compound has the structure:

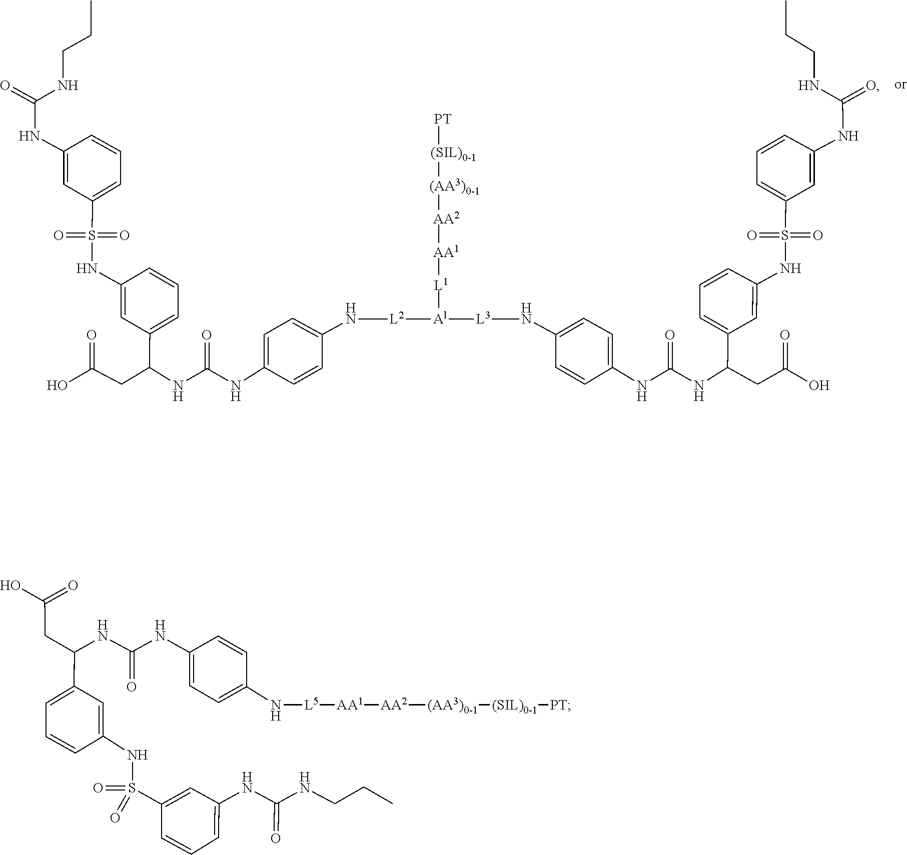

wherein:
PT is a PTEFb inhibitor;
each AA¹, AA², and AA³ is independently an amino acid,
SIL is a self-immolative linker (e.g., a PABC linker);
L¹, L², L³, and L⁵ are bivalent linkers,
A¹ is a trivalent linker; and
IN is an integrin binder.

In still another aspect, described herein is a compound, or a pharmaceutically acceptable salt thereof, or a stereoisomer or mixture of stereoisomers thereof, or a pharmaceutical composition thereof, for use as a medicament. In still another aspect, described herein is a compound, or a pharmaceutically acceptable salt thereof; or a stereoisomer or mixture of stereoisomers thereof, or a pharmaceutical composition thereof, for use in a method of treating a disease or disorder disclosed herein. In some embodiments, the disease or disorder is a hyperproliferative disorder. In some embodiments, the disease or disorder is a cancer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows the stability of a representative compound in rat plasma at 37° C. for 4 h.

FIG. 2 shows the stability of a representative compound in human plasma at 37° C. for 4 h.

FIG. 3 shows the stability of a representative compound in buffer pH 7.4 at 37° C. for 24 h.

FIG. 4 shows detection of metabolite from Compounds 7 and 12 in the absence of cathepsin B (control).

FIG. 5 shows detection of metabolite after incubating Compounds 7 and 12 with cathepsin B (test).

FIG. 6 shows detection of metabolite from Compounds 8 and 13 in the absence of legumain (control).

FIG. 7 shows detection of metabolite after incubating Compounds 8 and 13 with legumain (test).

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Existing chemotherapy causes serious side effects due to toxic action of administered chemotherapeutics on proliferating cells of other normal tissues. In the past, various attempts to improve the selectivity of chemotherapeutics have been made, including, the synthesis of prodrugs which could be released more or less selectively in the target tissue, for example, by change of the pH (DE-A 42 29903), by enzymes (EP-A 511917 and EP-A 595133), or by antibody-enzyme conjugates (WO 88/07378; U.S. Pat. No. 4,975,278; and EP-A 595133). However, some of the issues with these approaches include the lack of stability of the conjugates in other tissues and organs and the ubiquitous distribution of the active compounds, resulting in extracellular release of the active compounds in the tumor tissue. To address these issues, Ruoslahti et al. demonstrated conjugates on which a tumor-targeting molecule is linked to a functional unit, such as, a cytostatic or a detectable label (WO 1998010795). The linkage was carried out such that the integrin antagonists (e.g., peptides possessing a RGD sequence (arginine-glycine-aspartate) and the functional unit were directly bonded to one another with retention for their respective properties. Although these conjugates displayed a higher concentration selectively in the immediate vicinity of tumor cells by binding of the entity addressing a tumor, the cytostatic could not be released into the intracellular space of the tumor tissue.

In other studies, examples of conjugates composed of a cleavable linker(s) in contact with enzymes (e.g., anti-turmo prodrug selectively cleavable by collagenase (IV) and elastase (WO 00/69472), conjugates of Auristatins linked to an αᵥβ₃ integrin targeting moiety via a legumain-cleavable linker (Y. Liu et al. Mol. Pharm. 2012, 9, 168), and conjugates of a cytotoxic agent and a peptide linker which can be cleaved by elastase (EP 1 238 678) were disclosed. Gennari et al describe taxane derivatives linked to a cyclopeptidic RGD integrin recognition motif and a diketopiperazine (DKP) scaffold via elastase cleavable linker containing a self-immolative spacer unit (Chem. Eur. J. 2019, 25, 1696-1700). However, particular challenges of such conjugates included poor solubility in intravenous administration in appropriate vehicles; inefficient tumor penetration of intact conjugates; low stability in plasma enough to avoid systemic de-conjugation; inefficient binding to the targeted receptors in tumor microenvironment; inefficient cleavage by enzymes present in tumor microenvironment, and low stability and cellular permeability of cleaved toxophore moieties enough to enhance tumor cell uptake versus re-distribution.

The invention disclosed herein relates to novel conjugates of a binder or a derivative thereof with one or more molecules of an active component, such as, CDK9 kinase inhibitor, which is conjugated to the binder via a enzymatically-cleavable linker, which can release the active component extracellularly in the tumor microenvironement, and methods for their preparation, their use for the treatment or prophylaxis of disorders, in particular, of hyper-proliferative disorders.

### Compounds

Cancer cells overexpress certain enzymes, such as, serine proteases, including neutrophil elastase, and cysteine proteases including legumain, or cathepsin B. Conjugates described herein are enzymatically cleavable by a serine protease or cysteine protease, including neutrophil elastase, legumain, or cathepsin. In some embodiments, conjugates described herein are selectively cleavable in the microenvironment of cancer cells, with less cleavage taking place in the circulation or in healthy tissues. In some embodiments, a conjugate described herein is an anti-cancer compound after activation by a tumor-associated enzyme, such as, neutrophil elastase, legumain, or cathepsin B. In some embodiments, a conjugate described herein is non-toxic, in therapeutic concentrations, in the absence of said activation. The anti-cancer compounds described herein are inhibitors of PTEFb or CDK9. These agents may also be conjugated, e.g., via an enzymatically cleavable linker or a spacer, to an integrin binder. Integrin binders for use in the present disclosure include any agent that can bind to an integrin receptor (e.g., an αᵥβ₃ integrin receptor). Examples include, but are not limited to, small molecules, peptides, proteins, and the like. In some embodiments, the integrin binder is tumor binding moiety. In some embodiments, the integrin binding moiety binds to an integrin receptor that is alpha-v beta-3 (or alternatively: αᵥβ₃, aᵥb₃, or even avb3). In some embodiments, a conjugate described herein acts as a prodrug, e.g., an enzymatically cleavable prodrug, and one of the cleaved components is a PTEFb inhibitor described herein.

In some embodiments, provided herein is a compound, or a method of treating a disease (e.g., a CDK9/PTEFb-related disease) with a compound, the compound comprising a sulfoximine-linked CDK9/PTEFb inhibitor conjugated to an integrin binder via an enzymatically-cleavable linker.

In some embodiments, the enzymatically-cleavable linker comprises (i) a peptide that is cleavable by a disease-associated enzyme (e.g., neutrophil elastase, legumain, or cathepsin B); and/or (ii) a polymeric spacer (e.g., a polyethylene glycol (PEG) or polyethylenimine (PEI) spacer). In some embodiments, the peptide is bonded to the active agent (e.g., PTEFb inhibitor) via a sulfoximine linkage. In some embodiments, the peptide and active agent are conjugated via a self-immolative linker. In some embodiments, the disease-associated enzyme is a cancer-associated enzyme. In some embodiments, the disease-associated enzyme is a protease. In some embodiments, the disease-associated enzyme is a tumor-associated protease. In some embodiments, the enzymatically-cleavable linker comprises (i) a peptide that is cleavable by a disease-associated enzyme (e.g., neutrophil elastase, legumain, or cathepsin B); (ii) one or more (e.g., 1 to 10) polymeric spacers (e.g., polyethylene glycol (PEG) or polyethylenimine (PEI) spacers, or a combination thereof); and (iii) one or more branching units (e.g., an amino acid or a derivative thereof such as described herein in A¹).

In an aspect, provided herein is a compound, or a pharmaceutically acceptable salt thereof; or a stereoisomer or mixture of stereoisomers thereof, having a structure represented by any one of Formulae (01)-(05):

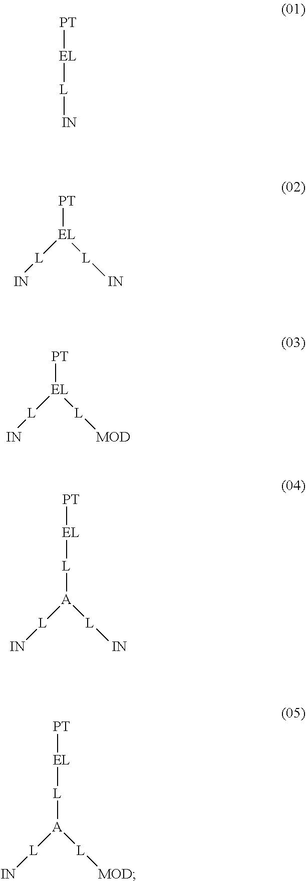

wherein:
PT is a PTEFb inhibitor;
EL is an enzymatically-cleavable linker;
L is a stable linker;
A is a trivalent linker;
IN is an integrin binder; and
MOD is a physicochemical or pharmacokinetic modulator.

In some embodiments, EL is a dipeptide having the formula -AA¹-AA²-, or a tripeptide having the formula -AA¹-AA²-AA³-, wherein each AA¹, AA², and AA³ is independently an amino acid, or a derivative thereof. In some embodiments, EL further comprises a self-immolative linker (SIL). In some embodiments, provided herein is a compound of any one of Formulae (01)-(05), wherein EL has the formula:

*-AA¹-AA²-(AA³)₀₋₁-(SIL)₀₋₁-**;
wherein:
*is a bond to L (e.g., L¹, L⁴, L⁵, L⁶, or L⁷);
** is a bond to PT;
each AA¹, AA², and AA³ is independently an amino acid; and SIL is a self-immolative linker.

In some embodiments, EL is:

*-AA¹-AA²-AA³-SIL-**, *-AA¹-AA²-AA³-**,

*-AA¹-AA²-SIL-**, or *-AA¹-AA²**;

wherein * is a bond to L (e.g., L¹, L⁴, L⁵, L⁶, or L⁷); ** is a bond to PT; AA¹, AA², and AA³ are each independently an amino acid, or a derivative thereof, and SIL is a self-immolative linker.

In some embodiments, EL is enzymatically cleavable. In some embodiments, EL is enzymatically cleavable and SIL is absent. In some embodiments, EL is not enzymatically cleavable. In some embodiments, EL is not enzymatically cleavable, and SIL is present. In some embodiments, SIL is

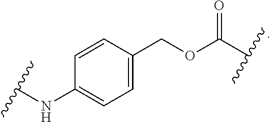 [SMILES: *Nc1ccc(COC(*)=O)cc1]

In another aspect, provided herein is a compound, or a pharmaceutically acceptable salt thereof; or a stereoisomer or mixture of stereoisomers thereof, having a structure represented by Formula (A), Formula (C), or Formula (E):

PT-EL-L¹-A¹(L²-IN)(L³-IN) Formula (A)

PT-EL-L⁵-IN Formula (C)

PT-EL-L¹-A¹(L²-IN)(L³-MOD) Formula (E)
wherein, in each instance:
PT is a monovalent radical of a PTEFb inhibitor (e.g., a sulfoximine-containing (e.g., sulfoximine-linked) small molecule PTEFb inhibitor (e.g., a macrocyclic PTEFb inhibitor));
EL is an enzymatically-cleavable linker (e.g., an elastase-cleavable linker);
L¹, L², L³, and L⁵ are each bivalent linkers (e.g., substituted or unsubstituted C₁₋₃₀ alkyl, substituted or unsubstituted heteroalkyl, substituted or unsubstituted aralkyl, substituted or unsubstituted heteroaralkyl, substituted or unsubstituted heteroalkyl-aryl, or substituted or unsubstituted heteroalkyl-heteroaryl linkers);
IN is in each instance, an integrin binder;
MOD is a physicochemical or pharmacokinetic modulating group (e.g., a polar or ionizable group (e.g., an amine or a carboxylic acid)); and
A¹ is a trivalent linker (e.g., a trivalent radical containing 1 to 100 non-hydrogen atoms, optionally containing alkyl, heteroalkyl, carbocyclic, and/or heterocyclic groups, or any combination thereof).

In some embodiments, EL is cleavable by neutrophil elastase. In some embodiments, EL is a tripeptide, having the formula -AA¹-AA²-AA³-, wherein each AA¹, AA², and AA³ is independently an amino acid (including N-alkyl amino acids). Each of AA¹, AA², and AA³ as used herein can be any naturally occurring or modified amino acid known in the art. For example, in some embodiments, each of AA¹, AA², and AA³ is selected from Ala, Arg, Asn, Asp, Cys, Glu, Gln, Gly, His, Ile, Leu, Lys, Met, Phe, Pro, Ser, Thr, Trp, Tyr, Val, Abu (2-aminobutyric acid, or homoalanine), Nva (norvaline), Nle (norleucine), Orn (ornithine), and Cit (citrulline).

In some embodiments, AA¹ is Abu, Ala, Asp, Asp*, Asn, Glu, Glu* Gly, His, or Nva. As used herein, Asp* or Glu* indicate that the side-chain carboxylic acid is modified with an ester prodrug (e.g., an alkylamine ester or a heteroalkyl-IN ester, defined herein as R¹). In some embodiments, AA¹ is L-Abu, L-Ala, L-Asp, L-Asp*, L-Asn, L-Glu, L-Glu* L-Gly, L-His, or L-Nva. In some embodiments, AA² is Pro. In some embodiments, AA² is L-Pro. In some embodiments, AA³ is Ile, Leu, Val, or Ala. In some embodiments, AA³ is L-Ile, L-Leu, L-Val, or L-Ala. In some embodiments, EL is a tripeptide having the formula: -L-Asp-L-Pro-L-Ala, -L-Asp-L-Pro-L-Val, -L-Asn-L-Pro-L-Val, -Gly-L-Pro-L-Val-, -L-Ala-L-Pro-L-Val-, -L-Nva-L-Pro-L-Val-, -L-His-L-Pro-L-Val-, -L-Asp-L-Pro-L-Ile, -L-Asn-L-Pro-L-Ile, -Gly-L-Pro-L-Ile-, -L-Ala-L-Pro-L-Ile-, -L-Nva-L-Pro-L-Ile-, -L-His-L-Pro-L-Ile-, -L-Asp-L-Pro-L-Leu, -L-Asn-L-Pro-L-Leu, -Gly-L-Pro-L-Leu-, -L-Ala-L-Pro-L-Leu-, -L-Nva-L-Pro-L-Leu-, or -L-His-L-Pro-L-Leu-.

In some embodiments, EL is a tripeptide having the formula Gly-L-Pro-L-Val, L-Asp-L-Pro-L-Val or L-Asn-L-Pro-L-Val. In some embodiments, EL is a tripeptide having the formula L-Asp-L-Pro-L-Val or L-Asn-L-Pro-L-Val. In some embodiments, EL is a tripeptide having the formula Gly-L-Pro-L-Val. In some embodiments, EL is a tripeptide having the formula L-Asp*-L-Pro-L-Val. In some embodiments, EL is a tripeptide having the formula L-Asp-L-Pro-L-Val. (i.e., “EL-1a”). In some embodiments, EL is a tripeptide having the formula L-Asn-L-Pro-L-Val (i.e., “EL-1b”).

In some embodiments, EL is a tripeptide having the formula L-Glu-L-Pro-L-Val. In some embodiments, EL is a tripeptide having the formula L-Glu*-L-Pro-L-Val. As used herein, Asp* or Glu* indicate the side-chain carboxylic acid is modified with an ester prodrug (e.g., an alkylamine ester or a heteroalkyl-IN ester).

In some embodiments, provided herein is a compound, or a pharmaceutically acceptable salt thereof; or a stereoisomer or mixture of stereoisomers thereof, having a structure represented by Formula (I) or Formula (I′):

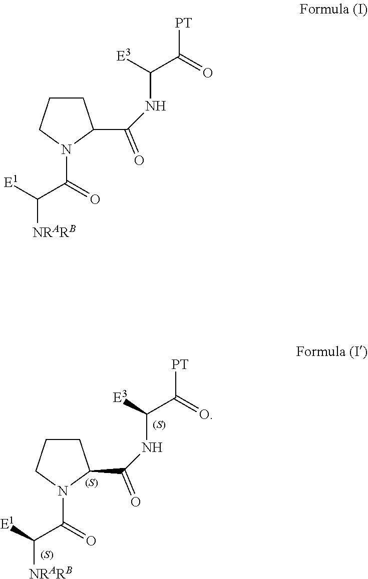

In some embodiments, provided herein is a compound, or a pharmaceutically acceptable salt thereof; or a stereoisomer or mixture of stereoisomers thereof, having a structure represented by Formula (I) or Formula (I′):

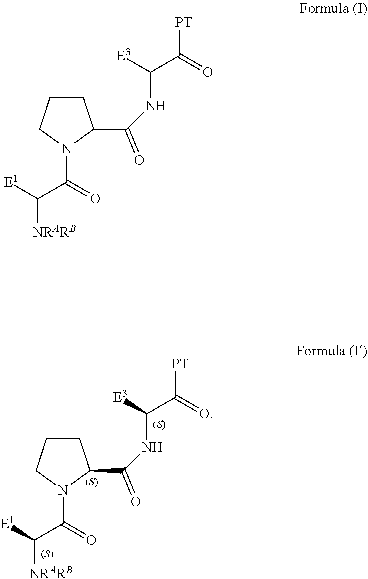

wherein:
PT is a monovalent radical of a PTEFb inhibitor;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
E³ is —CH₃, CH(CH₃)₂, CH₂CH(CH₃)₂, or CH(CH₃)CH₂CH₃;
R^{A} is hydrogen or C₁₋₆ alkyl;
R^{B} is -L¹-A¹(L²-(IN))(L³-(IN)), L⁵-IN, or -L¹-A¹(L²-(IN))(L³-(MOD));
   wherein:
   MOD is a physicochemical or pharmacokinetic modulating group;
   IN is an integrin binder;
   A¹ is a trivalent linker (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl));
   each of L¹, L², L³, and L⁵, is independently a bivalent linker (e.g., a substituted or unsubstituted C₁₋₆₀ alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker);
   R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl.

In some embodiments, provided herein is a compound, or a pharmaceutically acceptable salt thereof; or a stereoisomer or mixture of stereoisomers thereof, having a structure of Formula (I-A) or Formula (I-A′):

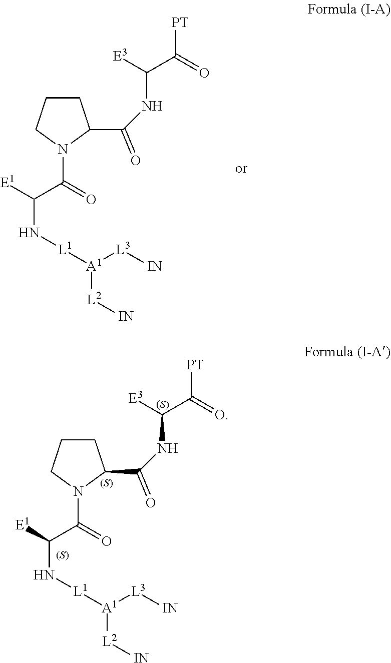

In some embodiments, provided herein is a compound, or a pharmaceutically acceptable salt thereof; or a stereoisomer or mixture of stereoisomers thereof, having a structure of Formula (I-A) or Formula (I-A′), wherein:
PT is a monovalent radical of a PTEFb inhibitor;
E³ is —CH₃, —CH(CH₃)₂, —CH₂CH(CH₃)₂, or —CH(CH₃)CH₂CH₃;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
IN is in each instance, independently, a monovalent radical of an integrin binder;
A¹ is a trivalent linker (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl));
each of L¹, L², and L³ is independently a substituted or unsubstituted C₁₋₃₀ alkyl, or substituted or unsubstituted heteroalkyl; and
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl.

In some embodiments, provided herein is a compound, or a pharmaceutically acceptable salt thereof; or a stereoisomer or mixture of stereoisomers thereof, having a structure of Formula (I-C):

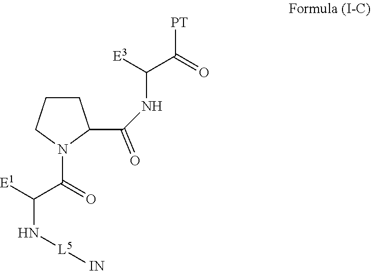

wherein E³ is —CH₃, —CH(CH₃)₂, —CH₂CH(CH₃)₂, or —CH(CH₃)CH₂CH₃.

In some embodiments, E³ is —CH(CH₃)₂. In some embodiments, E³ is —CH₂CH(CH₃)₂. In some embodiments, E³ is —CH(CH₃)CH₂CH₃. In some embodiments, E³ is —CH₃.

In some embodiments, provided herein is a compound, or a pharmaceutically acceptable salt thereof; or a stereoisomer or mixture of stereoisomers thereof, having a structure of Formula (I-C) or Formula (I-C′):

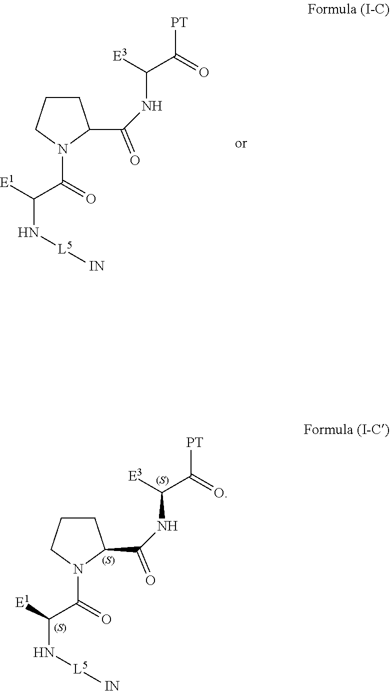

In some embodiments, provided herein is a compound, or a pharmaceutically acceptable salt thereof; or a stereoisomer or mixture of stereoisomers thereof, having a structure of Formula (I-C) or Formula (I-C′):

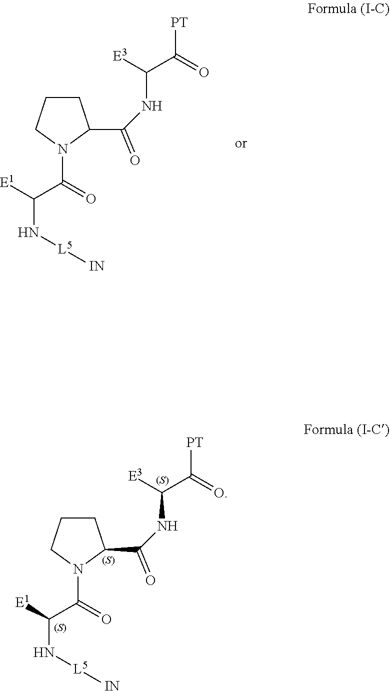

wherein:
PT is a monovalent radical of a PTEFb inhibitor;
E³ is —CH₃, —CH(CH₃)₂, —CH₂CH(CH₃)₂, or —CH(CH₃)CH₂CH₃;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
IN is in each instance, independently, a monovalent radical of an integrin binder;
L⁵ is a substituted or unsubstituted alkyl, or substituted or unsubstituted heteroalkyl;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
wherein; L⁶ is a substituted or unsubstituted C₁₋₃₀ alkyl, or substituted or unsubstituted heteroalkyl.

In some embodiments, provided herein is a compound of Formula (I-C) or Formula (I-C′), or a pharmaceutically acceptable salt thereof, or a stereoisomer or mixture of stereoisomers thereof, wherein L⁵ is a linker having a structure represented by the formula:

—(CO)ₘ(CH₂)ₙ(OC₂₋₆ alkyl)ₒ(NH)ₚ(CO)_{q}—; (i)

—(CO)ᵣ(CH₂)ₛ(NR¹⁰C₂₋₆ alkyl)ₜ(NR¹¹)ᵤ(CO)ᵥ—; or (ii)

—(CO)ᵣ(CH₂)ₛ(NR¹⁰C(O)C₁₋₆ alkyl)ₜ(NR¹¹)ᵤ(CO)ᵥ—; (iii)
wherein:
R¹⁰ is, in each instance, independently selected from hydrogen or C₁₋₃ alkyl;
R¹¹ is, in each instance, independently selected from hydrogen or C₁₋₃ alkyl;
m is 0 or 1;
n is 0 to 10;
o is 1 to 10;
p is 0 or 1;
q is 0 or 1;
r is 0 or 1;
s is 0 to 10;
t is 1 to 10;
u is 0 or 1; and
v is 0 or 1.

In some embodiments, provided herein is a compound, or a pharmaceutically acceptable salt thereof; or a stereoisomer or mixture of stereoisomers thereof, having a structure of Formula (I-C1), Formula (I-C2), or Formula (I-C3):

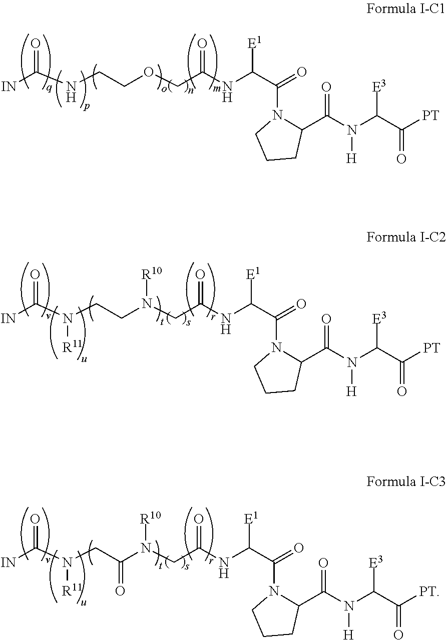

In some embodiments, provided herein is a compound, or a pharmaceutically acceptable salt thereof; or a stereoisomer or mixture of stereoisomers thereof, having a structure of Formula (I-C1) wherein:
R¹⁰ is —CH₃;
R¹¹ is hydrogen or —CH₃;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
E³ is —CH₃, —CH(CH₃)₂, —CH₂CH(CH₃)₂, or —CH(CH₃)CH₂CH₃;
R¹ is C₁₋₆ alkyl substituted with —NH₂, —N(CH₃)₂, or —N(CH₃)₃⁺;
IN is a monovalent radical of an integrin binder
m is 1;
n is 2 to 4;
o is 1 to 6;
p is 1;
q is 1;
r is 1;
s is 1 to 4;
t is 1 to 6;
u is 1; and
v is 1.

In some embodiments, provided herein is a compound, or a pharmaceutically acceptable salt thereof; or a stereoisomer or mixture of stereoisomers thereof, having a structure of Formula (I-E) or Formula (I-E′):

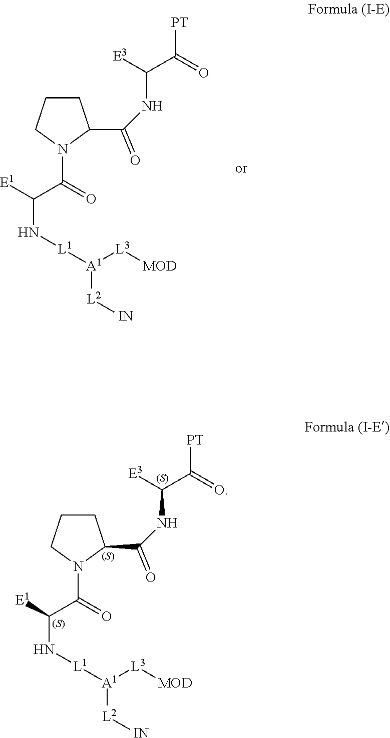

In some embodiments, provided herein is a compound, or a pharmaceutically acceptable salt thereof; or a stereoisomer or mixture of stereoisomers thereof, having a structure of Formula (I-E) or Formula (I-E′), wherein:
PT is a PTEFb inhibitor;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
E³ is —CH₃, —CH(CH₃)₂, —CH₂CH(CH₃)₂, or —CH(CH₃)CH₂CH₃;
IN is an integrin binder;
MOD is a physicochemical or pharmacokinetic modulating group;
A¹ is a trivalent linker (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl));
L¹, L², and L³, are each bivalent linkers (e.g., a substituted or unsubstituted C₁₋₃₀ alkyl, or substituted or unsubstituted heteroalkyl);
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl.

In some embodiments, provided herein is a compound, or a pharmaceutically acceptable salt thereof; or a stereoisomer or mixture of stereoisomers thereof, having a structure of Formula (II-A):

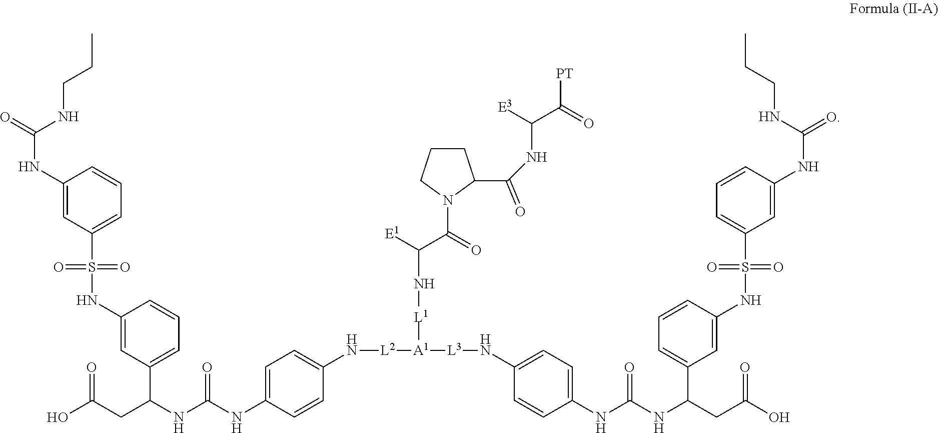

In some embodiments, provided herein is a compound, or a pharmaceutically acceptable salt thereof; or a stereoisomer or mixture of stereoisomers thereof, having a structure of Formula (II-A), wherein:
PT is a monovalent radical of a PTEFb inhibitor;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
E³ is —CH₃, —CH(CH₃)₂, —CH₂CH(CH₃)₂, or —CH(CH₃)CH₂CH₃;
A¹ is a trivalent linker (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl));
each of L¹, L², and L³ is independently a bivalent linker (e.g., a substituted or unsubstituted C₁₋₆₀ alkyl, or substituted or unsubstituted 1-60 heteroalkyl, optionally substituted or intersected by heteroaryl);
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
wherein:
L⁶ is a substituted C₁₋₃₀ alkyl, or substituted or unsubstituted heteroalkyl; and
IN is a monovalent radical of an integrin binder.

In some embodiments, provided herein is a compound, or a pharmaceutically acceptable salt thereof; or a stereoisomer or mixture of stereoisomers thereof, having a structure of Formula (II-A), wherein:
PT is a monovalent radical of a PTEFb inhibitor;
E¹ is hydrogen, —CH₂C(O)NH₂, —CH₂C(O)OH, —CH₂C(O)OR¹;
E³ is —CH₃, —CH(CH₃)₂, —CH₂CH(CH₃)₂, or —CH(CH₃)CH₂CH₃;
R¹ is C₁₋₆ alkyl substituted with —NHL⁶-IN, —N(C₁₋₆alkyl)₂, or —N(C₁₋₆alkyl)₃⁺;
L¹ is —C(O)—C₂₋₆ alkyl-[O—C₂₋₆ alkyl]₁₋₈-NH*,
   —C(O)—C₁₋₆ alkyl-[N(CH₃)—C₂₋₆ alkyl]₁₋₈-NH—*,
   —C(O)—C₁₋₆ alkyl-[N(CH₃)—C₂₋₆ alkyl]₁₋₈-N(CH₃)—*;
   —C(O)—C₁₋₆ alkyl-[N(CH₃)C(O)—C₁₋₆ alkyl]₁₋₈-NH—*; or
   —C(O)—C₁₋₆ alkyl-[N(CH₃)C(O)—C₁₋₆ alkyl]₁₋₈-N(CH₃)—*;
A¹ is *C(O)—C₀₋₆ alkyl-Y(C₀₋₆ alkyl-NH**)(C₀₋₆ alkyl-NH***),
   C(O)—C₀₋₆ alkyl-C(O)NH—Y(C₀₋₆ alkyl-NH**)(C₀₋₆ alkyl-NH**),
   C(O)—C₀₋₆ alkyl-C(O)NH—Y(C₀₋₆ alkyl-NH**)(C₀₋₆ alkyl-C(O)***),
   *C(O)—C₀₋₆ alkyl-C(O)NH—Y(C₀₋₆ alkyl-C(O)**)(C₀₋₆ alkyl-C(O)***), or
   *C(O)—C₀₋₆ alkyl-C(O)NH—Y(C₀₋₆ alkyl-C(O)NH—C₀₋₆ alkyl-NH**)(C₀₋₆ alkyl-C(O)NH—C₀₋₆ alkyl-NH***);
   wherein:
   Y is CH or N;
   *is a bond between L¹ and A¹;
   ** is a bond between A¹ and L²;
   *** is a bond between A¹ and L³;
L² is C(O)—,
   **C(O)—C₁₋₆ alkyl-NHC(O)—,
   **C(O)—C₁₋₆ alkyl-[O—C₂₋₆ alkyl]₁₋₈-NHC(O)—,
   **C(O)—C₁₋₆ alkyl-[N(CH₃)—C₂₋₆ alkyl]₁₋₈-NHC(O)—,
   **C(O)—C₁₋₆ alkyl-[N(CH₃)—C₂₋₆ alkyl]₁₋₈-N(CH₃)C(O)—;
   **C(O)—C₁₋₆ alkyl-[N(CH₃)C(O)—C₁₋₆ alkyl]₁₋₈-NHC(O)—; or
   **C(O)—C₁₋₆ alkyl-[N(CH₃)C(O)—C₁₋₆ alkyl]₁₋₈-N(CH₃)C(O)—;
L³ is C(O)—,
   ***C(O)—C₁₋₆ alkyl-NHC(O)—,
   ***C(O)—C₁₋₆ alkyl-[O—C₂₋₆ alkyl]₁₋₈-NHC(O)—,
   ***C(O)—C₁₋₆ alkyl-[N(CH₃)—C₂₋₆ alkyl]₁₋₈-NHC(O)—,
   ***C(O)—C₁₋₆ alkyl-[N(CH₃)—C₂₋₆ alkyl]₁₋₈-N(CH₃)C(O)—;
   ***C(O)—C₁₋₆ alkyl-[N(CH₃)C(O)—C₁₋₆ alkyl]₁₋₈-NHC(O)—; or
   ***C(O)—C₁₋₆ alkyl-[N(CH₃)C(O)—C₁₋₆ alkyl]₁₋₈-N(CH₃)C(O)—; and
L⁶ is —C(O)—.

In some embodiments, provided herein is a compound, or a pharmaceutically acceptable salt thereof; or a stereoisomer or mixture of stereoisomers thereof, having a structure of Formula (II-A), wherein:
E¹ is hydrogen, —CH₂C(O)NH₂ or —CH₂C(O)OH;
E³ is —CH₃ or —CH(CH₃)₂;
L¹ is —C(O)—C₂₋₄ alkyl-[O—C₂₋₄ alkyl]₂₋₄-NH*,
   —C(O)—C₁₋₄ alkyl-[N(CH₃)—C₂₋₄ alkyl]₂₋₄-NH—*,
   —C(O)—C₁₋₄ alkyl-[N(CH₃)—C₂₋₄ alkyl]₂₋₄-N(CH₃)—*;
A¹ is *C(O)—CH(NH**)(C₁₋₄ alkyl-NH***), or
   *C(O)—C₁₋₄ alkyl-C(O)NH—CH(C₁₋₄ alkyl-C(O)NH—C₂₋₄ alkyl-NH**)(C(O)NH—C₂₋₄ alkyl-NH***);
L² is **C(O)—;
   **C(O)—C₂₋₄ alkyl-NHC(O)—,
   **C(O)—C₂₋₄ alkyl-[O—C₂₋₆ alkyl]₂₋₄-NHC(O)—,
   **C(O)—C₁₋₄ alkyl-[N(CH₃)—C₂₋₆ alkyl]₂₋₄-NHC(O)—, or
   **C(O)—C₁₋₄ alkyl-[N(CH₃)—C₂₋₆ alkyl]₂₋₄-N(CH₃)C(O)—; and
L³ is ***C(O)—,
   ***(C(O)—C₂₋₄ alkyl-NHC(O)—,
   ***C(O)—C₂₋₄ alkyl-[O—C₂₋₆ alkyl]₂₋₄-NHC(O)—,
   ***C(O)—C₁₋₄ alkyl-[N(CH₃)—C₂₋₆ alkyl]₂₋₄-NHC(O)—, or
   ***C(O)—C₁₋₄ alkyl-[N(CH₃)—C₂₋₆ alkyl]₂₋₄-N(CH₃)C(O)—.

In some embodiments, A¹ is an amino acid or a derivative thereof. In some embodiments, A¹ is an amino acid, or A¹ is an amino acid derivative comprising an amino acid, wherein the carboxylate group(s) is/are substituted with heteroalkyl groups. In some embodiments, A¹ is an amino acid derivative comprising an amino acid, wherein the carboxylate group(s) is/are substituted with C₁₋₆ aminoalkyl groups. In some embodiments, A¹ is an amino acid, or A¹ is an amino acid derivative comprising an amino acid, wherein the carboxylate group(s) is/are substituted with C₁₋₆ aminoalkyl groups. In some embodiments, A¹ is an amino acid derivative comprising an amino acid (e.g., Glu or Asp), wherein the carboxylate groups are substituted with C₁₋₆ aminoalkyl groups (e.g., beta-alanine groups). In some embodiments, A¹ is an amino acid. In some embodiments, A¹ is lysine. In some embodiments, A¹ is L-Lys. In some embodiments, A¹ is D-Lys. In some embodiments, A¹ is Glu, or an aminoalkyl derivative thereof. In some embodiments, A¹ is L-Glu or a beta-alanine-substituted derivative thereof. In some embodiments, A¹ is D-Glu or a beta-alanine-substituted derivative thereof.

In some embodiments, A¹ is *C(O)—CH(NH**)(C₁₋₄ alkyl-NH***), wherein * is a bond between L¹ and A¹; ** is a bond between A¹ and L²; and *** is a bond between A¹ and L³. In some embodiments, A¹ is

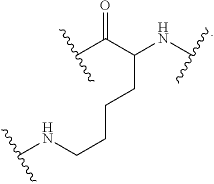 [SMILES: *NCCCCC(N*)C(*)=O]

In some embodiments, A¹ is or

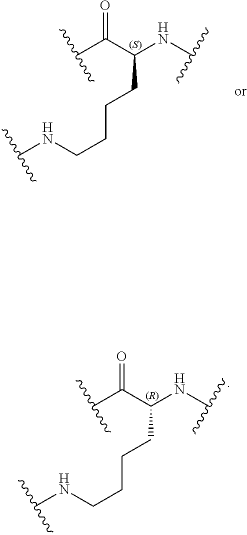

In some embodiments, A¹ is

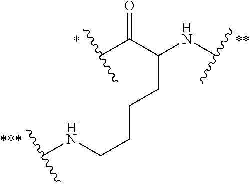 [SMILES: *NCCCCC(N*)C(*)=O]

wherein * is a bond between L¹ and A¹; ** is a bond between A¹ and L²; and *** is a bond between A¹ and L³. In some embodiments, A¹ is

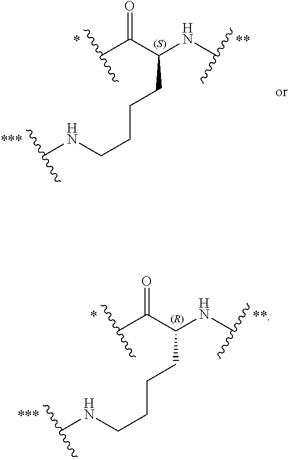

In some embodiments, A¹ is*C(O)—C₁₋₄ alkyl-C(O)NH—CH(C₁₋₄ alkyl-C(O)NH—C₁₋₄ alkyl-NH**)(C(O)NH—C₁₋₄ alkyl-NH***). In some embodiments, A¹ is H

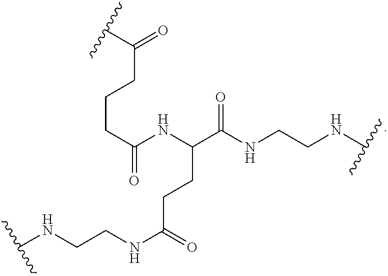 [SMILES: *NCCNC(=O)CCC(NC(=O)CCCC(*)=O)C(=O)NCCN*]

In some embodiments, A¹ is

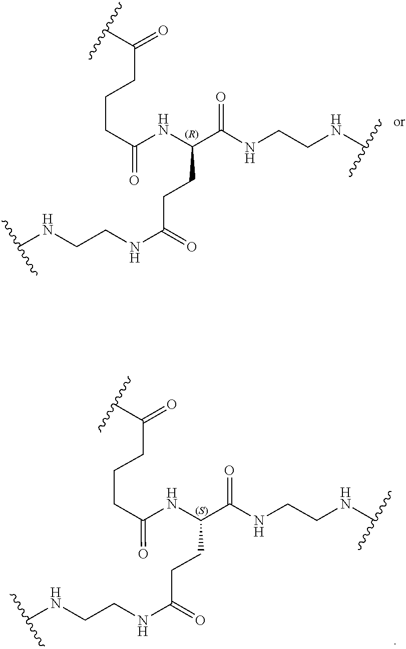

In some embodiments, A¹ is

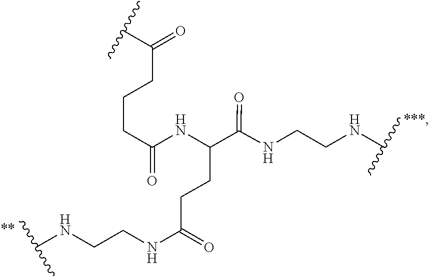 [SMILES: *NCCNC(=O)CCC(NC(=O)CCCC(*)=O)C(=O)NCCN*]

wherein * is a bond between L and A¹; ** is a bond between A¹ and L²; and *** is a bond between A¹ and L³. In some embodiments, A¹ is

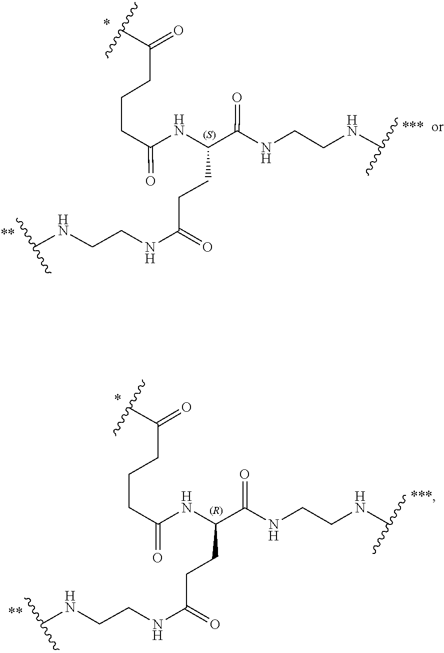

In some embodiments, provided herein is a compound, or a pharmaceutically acceptable salt thereof; or a stereoisomer or mixture of stereoisomers thereof, having a structure of Formula

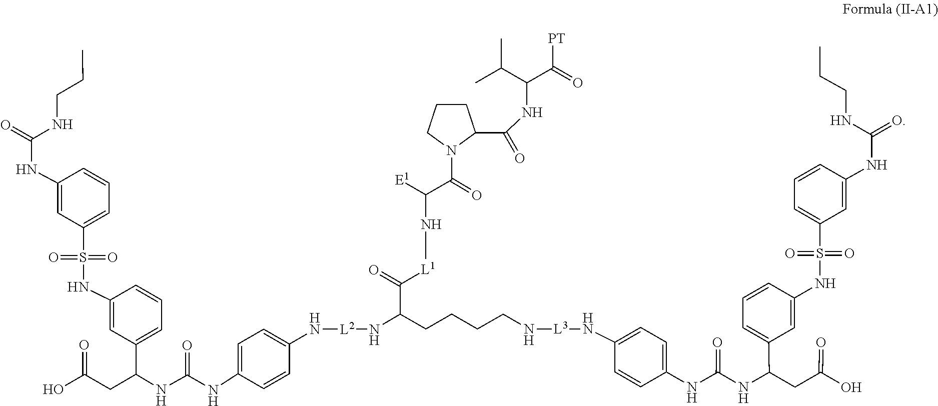

In some embodiments, provided herein is a compound, or a pharmaceutically acceptable salt thereof; or a stereoisomer or mixture of stereoisomers thereof, having a structure of Formula (II-A2):

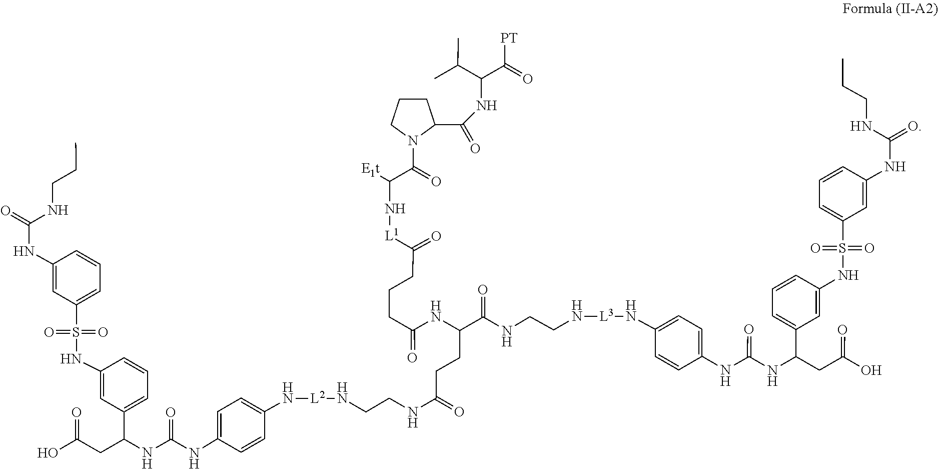

In some embodiments, E¹ is hydrogen, —CH₂C(O)NH₂, —CH₂C(O)OH, —CH₂C(O)OR¹. In some embodiments, E¹ is hydrogen, —CH₂C(═O)OH, or —CH₂C(═O)NH₂. In some embodiments, E¹ is —CH₂C(═O)OH or —CH₂C(═O)NH₂. In some embodiments, E¹ is —CH₂C(═O)OH. In some embodiments, E¹ is —CH₂C(═O)NH₂.

In some embodiments,
E¹ is —CH₂C(O)OH or —CH₂C(O)NH₂;
L¹ is —C(O)—C₂₋₄ alkyl-[O—C₂₋₄ alkyl]₂₋₄-NH*; or
   **C(O)—C₁₋₄ alkyl-[N(CH₃)—C₂₋₆ alkyl]₂₋₄-NHC(O)—,
A¹ is

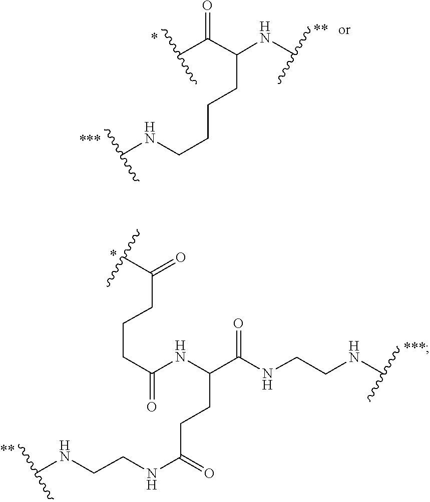 [SMILES: *NCCCCC(N*)C(*)=O ; *NCCNC(=O)CCC(NC(=O)CCCC(*)=O)C(=O)NCCN*]

L² is **C(O)—C₂₋₄ alkyl-[O—C₂₋₄ alkyl]₂₋₄-NHC(O); or
   **C(O)—C₁₋₄ alkyl-[N(CH₃)—C₂₋₆ alkyl]₂₋₄-NHC(O)—, and
L³ is ***C(O)—C₂₋₄ alkyl-[O—C₂₋₄ alkyl]₂₋₄-NHC(O); or
   **C(O)—C₁₋₄ alkyl-[N(CH₃)—C₂₋₆ alkyl]₂₋₄-NHC(O)—.

In some embodiments,
E¹ is —CH₂C(O)NH₂;
L¹ is —C(O)—CH₂—[N(CH₃)CH₂CH₂]₃—NH*;
A¹ is

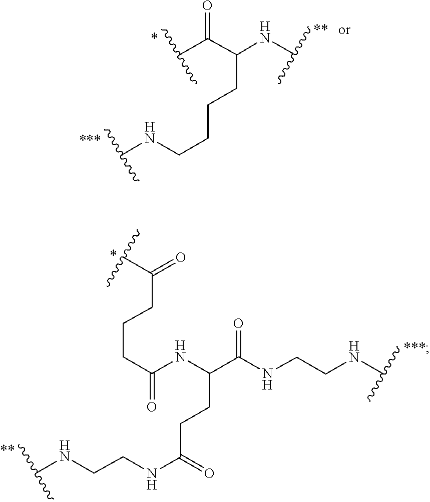 [SMILES: *NCCCCC(N*)C(*)=O ; *NCCNC(=O)CCC(NC(=O)CCCC(*)=O)C(=O)NCCN*]

L² is **C(O)—CH₂CH₂—[OCH₂CH₂]₃—NHC(O); and
L³ is ***C(O)—CH₂CH₂—[OCH₂CH₂]₃—NHC(O).

In some embodiments,
E¹ is —CH₂C(O)OH;
L¹ is —C(O)—CH₂CH₂—[OCH₂CH₂]₃—NH*;

A¹ is

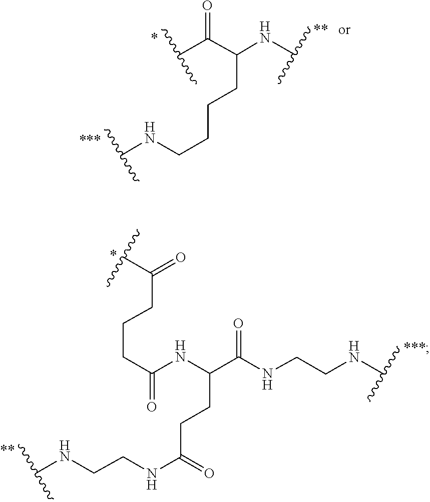 [SMILES: *NCCCCC(N*)C(*)=O ; *NCCNC(=O)CCC(NC(=O)CCCC(*)=O)C(=O)NCCN*]

L² is **C(O)—CH₂CH₂—[OCH₂CH₂]₃—NHC(O); and
L³ is ***C(O)—CH₂CH₂—[OCH₂CH₂]₃—NHC(O).

In some embodiments, provided herein is a compound, or a pharmaceutically acceptable salt thereof; or a stereoisomer or mixture of stereoisomers thereof, having a structure of Formula (II-C):

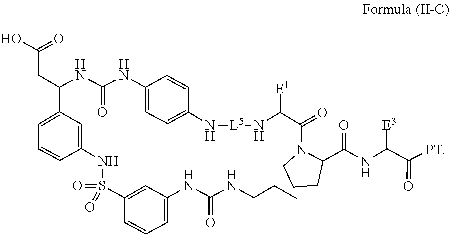

In some embodiments, provided herein is a compound of Formula (II-C), or a pharmaceutically acceptable salt thereof; or a stereoisomer or mixture of stereoisomers thereof, wherein:
PT is a monovalent radical of a PTEFb inhibitor;
E³ is —CH₃, —CH(CH₃)₂, —CH₂CH(CH₃)₂, or —CH(CH₃)CH₂CH₃;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
L⁵ is a bivalent linker (e.g., substituted or unsubstituted alkyl, or substituted or unsubstituted heteroalkyl;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
   wherein;
   L⁶ is a substituted C₁₋₃₀ alkyl, or substituted or unsubstituted heteroalkyl; and
   IN is a monovalent radical of an integrin binder.

In some embodiments, provided herein is a compound, or a pharmaceutically acceptable salt thereof; or a stereoisomer or mixture of stereoisomers thereof, wherein L⁵ is a linker having a structure represented by the formula:

—(CO)ₘ(CH₂)ₙ(OC₂₋₆ alkyl)ₒ(NH)ₚ(CO)_{q}— or (i)

—(CO)ᵣ(CH₂)ₛ(NR¹⁰C₂₋₆ alkyl)ₜ(NR¹¹)ᵤ(CO)ᵥ—; (ii)
wherein:
R¹⁰ is hydrogen or C₁₋₃ alkyl;
R¹¹ is hydrogen or C₁₋₃ alkyl;
m is 0 or 1;
n is 0 to 10;
o is 1 to 10;
p is 0 or 1;
q is 0 or 1;
r is 0 or 1;
s is 0 to 10;
t is 1 to 10;
u is 0 or 1; and
v is 0 or 1.

In some embodiments, provided herein is a compound, or a pharmaceutically acceptable salt thereof; or a stereoisomer or mixture of stereoisomers thereof, wherein:
R¹⁰ is hydrogen or —CH₃;
R¹¹ is hydrogen or —CH₃;
m is 0 or 1;
n is 2 to 4;
o is 1 to 8;
p is 0 or 1;
q is 0 or 1;
r is 0 or 1;
s is 2 to 4;
t is 1 to 8;
u is 0 or 1; and
v is 0 or 1.

In some embodiments, provided herein is a compound wherein:
L⁵ is a linker having a structure represented by the formula:

—(CO)ₘ(CH₂)ₙ(OC₂₋₆ alkyl)ₒ(NH)ₚ(CO)_{q};
wherein:
m is 0 or 1;
n is 2 to 4;
is 1 to 8;
p is 0 or 1;
q is 0 or 1;
In some embodiments, provided herein is a compound wherein:
L⁵ is a linker having a structure represented by the formula:

—(CO)ᵣ(CH₂)ₛ(NR¹⁰C₂₋₆ alkyl)ₜ(NR¹¹)ᵤ(CO)ᵥ—; or

—(CO)ᵣ(CH₂)ₛ(NR¹⁰C(O)C₁₋₆ alkyl)ₜ(NR¹¹)ᵤ(CO)ᵥ—;
wherein:
r is 0 or 1;
s is 1 to 4;
t is 1 to 8;
u is 0 or 1; and
v is 0 or 1.

In some embodiments, provided herein is a compound of Formula, or a pharmaceutically acceptable salt thereof; or a stereoisomer or mixture of stereoisomers thereof, wherein:
E¹ is hydrogen, —CH₂C(O)NH₂ or —CH₂C(O)OH;
L⁵ is a group of the formula: —C(O)—(CH₂)ₙ—(O—C₂₋₆ alkyl)ₒ-NHC(O)—.

In some embodiments, provided herein is a compound of Formula (II-C1):

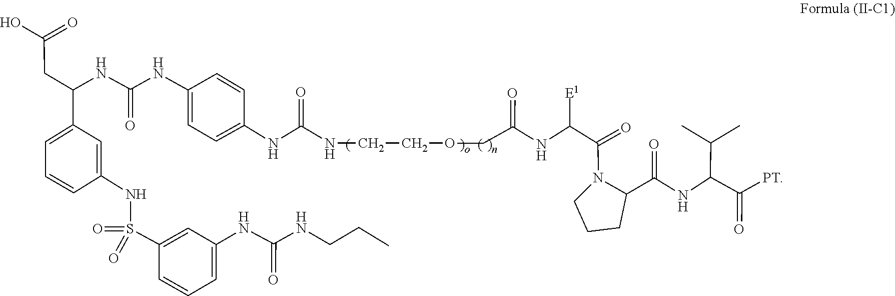

In some embodiments, provided herein is a compound of Formula (II-C1), wherein:
PT is a monovalent radical of a PTEFb inhibitor;
E¹ is hydrogen, —CH₂C(O)NH₂, —CH₂C(O)OH, —CH₂C(O)OR¹, —CH₂CH₂C(O)OH or —CH₂CH₂C(O)OR¹;
   R¹ is C₁₋₆alkyl substituted with —NH₂ or, —NHL⁶-IN, —N(CH₃)₂, or —N(CH₃)₃⁺;
   wherein:
   L⁶ is substituted C₁₋₃₀ alkyl, or substituted or unsubstituted heteroalkyl; and
   IN is a monovalent radical of an integrin binder;
   n is 2 to 4; and
   is 1 to 8.

In some embodiments, provided herein is a compound of Formula (II-C), wherein:
E¹ is hydrogen, —CH₂C(O)NH₂ or —CH₂C(O)OH;
L⁵ is a group of the formula: —C(O)—(CH₂)ₛ—(N(CH₃)—C₂₋₆ alkyl)ₜ-N(R¹¹)C(O)—.

In some embodiments, R¹¹ is hydrogen or —CH₃. In some embodiments, R¹¹ is hydrogen. In some embodiments, R¹¹ is —CH₃. In some embodiments, s is 1 to 10. In some embodiments, s is 1 to 8. In some embodiments, s is 1 to 4. In some embodiments, s is 1. In some embodiments, s is 2. In some embodiments, s is 3. In some embodiments, s is 4. In some embodiments, t is 1 to 10. In some embodiments, t is 1 to 8. In some embodiments, t is 2 to 6. In some embodiments, t is 2. In some embodiments, t is 3. In some embodiments, t is 4. In some embodiments, E¹ is —CH₂C(O)NH₂. In some embodiments, E¹ is —CH₂C(O)NH₂; R¹¹ is hydrogen or —CH₃; s is 1 to 4; and t is 2 to 6.

In some embodiments, provided herein is a compound, or a pharmaceutically acceptable salt thereof; or a stereoisomer or mixture of stereoisomers thereof, having a structure of Formula (II-C2):

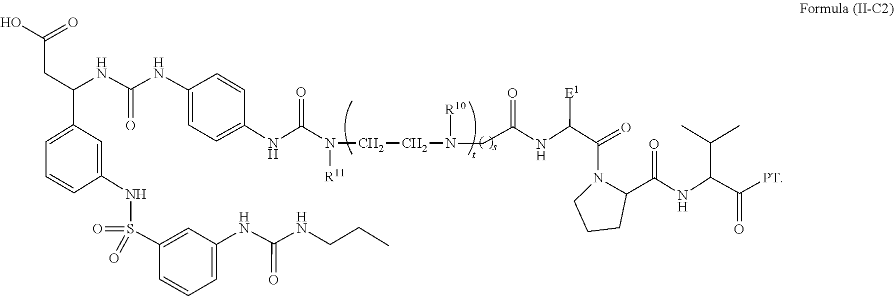

In some embodiments, provided herein is a compound of Formula (II-C2), or a pharmaceutically acceptable salt thereof; or a stereoisomer or mixture of stereoisomers thereof, wherein:
PT is a monovalent radical of a PTEFb inhibitor;
E¹ is hydrogen, —CH₂C(O)NH₂, —CH₂C(O)OH, —CH₂C(O)OR¹, —CH₂CH₂C(O)OH or —CH₂CH₂C(O)OR¹;
   R¹ is C₁₋₆alkyl substituted with —NH₂ or, —NHL⁶-IN, —N(CH₃)₂, or —N(CH₃)₃⁺;
   wherein:
   L⁶ is substituted C₁₋₃₀ alkyl, or substituted or unsubstituted heteroalkyl; and
   IN is a monovalent radical of an integrin binder;
R¹⁰ is hydrogen or —CH₃;
R¹¹ is hydrogen or —CH₃;
s is 1 to 4; and
t is 1 to 8.

In some embodiments, provided herein is a compound, or a pharmaceutically acceptable salt thereof; or a stereoisomer or mixture of stereoisomers thereof, having a structure of Formula (II-C3):

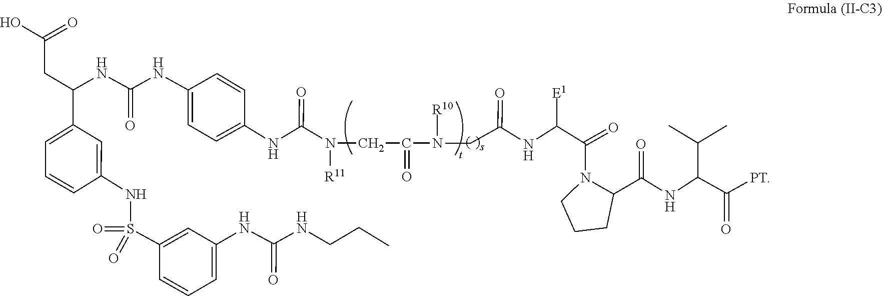

In some embodiments, provided herein is a compound of Formula (II-C3), or a pharmaceutically acceptable salt thereof; or a stereoisomer or mixture of stereoisomers thereof, wherein:
PT is a monovalent radical of a PTEFb inhibitor;
E¹ is hydrogen, —CH₂C(O)NH₂, —CH₂C(O)OH, —CH₂C(O)OR¹, —CH₂CH₂C(O)OH or —CH₂CH₂C(O)OR¹;
   R¹ is C₁₋₆alkyl substituted with —NH₂ or, —NHL⁶-IN, —N(CH₃)₂, or —N(CH₃)₃⁺;
   wherein:
   L⁶ is substituted C₁₋₃₀ alkyl, or substituted or unsubstituted heteroalkyl; and
   IN is a monovalent radical of an integrin binder;
R¹⁰ is hydrogen or —CH₃;
R¹¹ is hydrogen or —CH₃;
s is 1 to 4; and
t is 1 to 8.

In some embodiments, E¹ is —CH₂C(O)OR¹ and R¹ is a substituted or unsubstituted C₂-6alkyl. In some embodiments, R¹ is substituted C₂₋₆alkyl, wherein the alkyl is substituted with one or more groups independently selected from deuterium, halogen, —C₁₋₆alkyl, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), and oxo; wherein L⁶ is a substituted C₁₋₃₀ alkyl, or substituted or unsubstituted heteroalkyl. In some embodiments, R¹ is substituted C₂₋₆alkyl, wherein the alkyl is substituted with one or more groups independently selected from deuterium, halogen, —C₁₋₆alkyl, —CONH₂, —CONH(C₁₋₆alkyl), —NHL⁶-IN, —N(C₁₋₆alkyl)₂, —N(C₁₋₆alkyl)₃⁺, —NHCO(IN), and oxo; wherein L⁶ is —C(O)—. In some embodiments, R¹ is substituted C₂₋₆alkyl, wherein the alkyl is substituted with one or more groups independently selected from —NHL⁶-IN, —N(C₁₋₆alkyl)₂, —N(C₁₋₆alkyl)₃⁺; wherein L⁶ is —C(O)—. In some embodiments, R¹ is —C₂₋₆alkyl-NHC(O)—IN, —C₂₋₆alkyl-N(C₁₋₃alkyl)₂, or —C₂₋₆alkyl-N(C₁₋₃alkyl)_{3+.}

In some embodiments, R¹ is —C₂₋₆alkyl-NHC(O)—IN, —C₂₋₆alkyl-N(CH₃)₂, or —C₂₋₆alkyl-N(CH₃)₃⁺. In some embodiments, R¹ is —C₂₋₆alkyl-NHC(O)—IN. In some embodiments, R¹ is —C₂ alkyl-NHC(O)—IN. In some embodiments, R¹ is —C₃ alkyl-NHC(O)—IN. In some embodiments, R¹ is —C₄ alkyl-NHC(O)—IN. In some embodiments, R¹ is —C₅ alkyl-NHC(O)—IN. In some embodiments, R¹ is —C₆ alkyl-NHC(O)—IN. In some embodiments, R¹ is —C₂₋₆alkyl-N(CH₃)₂. In some embodiments, R¹ is —C₂ alkyl-N(CH₃)₂. In some embodiments, R¹ is —C₃ alkyl-N(CH₃)₂. In some embodiments, R¹ is —C₄ alkyl-N(CH₃)₂. In some embodiments, R¹ is —C₅ alkyl-N(CH₃)₂. In some embodiments, R¹ is —C₆ alkyl-N(CH₃)₂. In some embodiments, R¹ is —C₂₋₆alkyl-N(CH₃)₃⁺. In some embodiments, R¹ is —C₂ alkyl-N(CH₃)₃⁺. In some embodiments, R¹ is —C₃ alkyl-N(CH₃)₃⁺. In some embodiments, R¹ is —C₄ alkyl-N(CH₃)₃⁺. In some embodiments, R¹ is —C₅ alkyl-N(CH₃)₃⁺. In some embodiments, R¹ is —C₆ alkyl-N(CH₃)₃⁺.

In some embodiments, provided herein is a compound, or a pharmaceutically acceptable salt thereof; or a stereoisomer or mixture of stereoisomers thereof, having a structure of Formula (II-C1), Formula (II-C2), or Formula (II-C3):

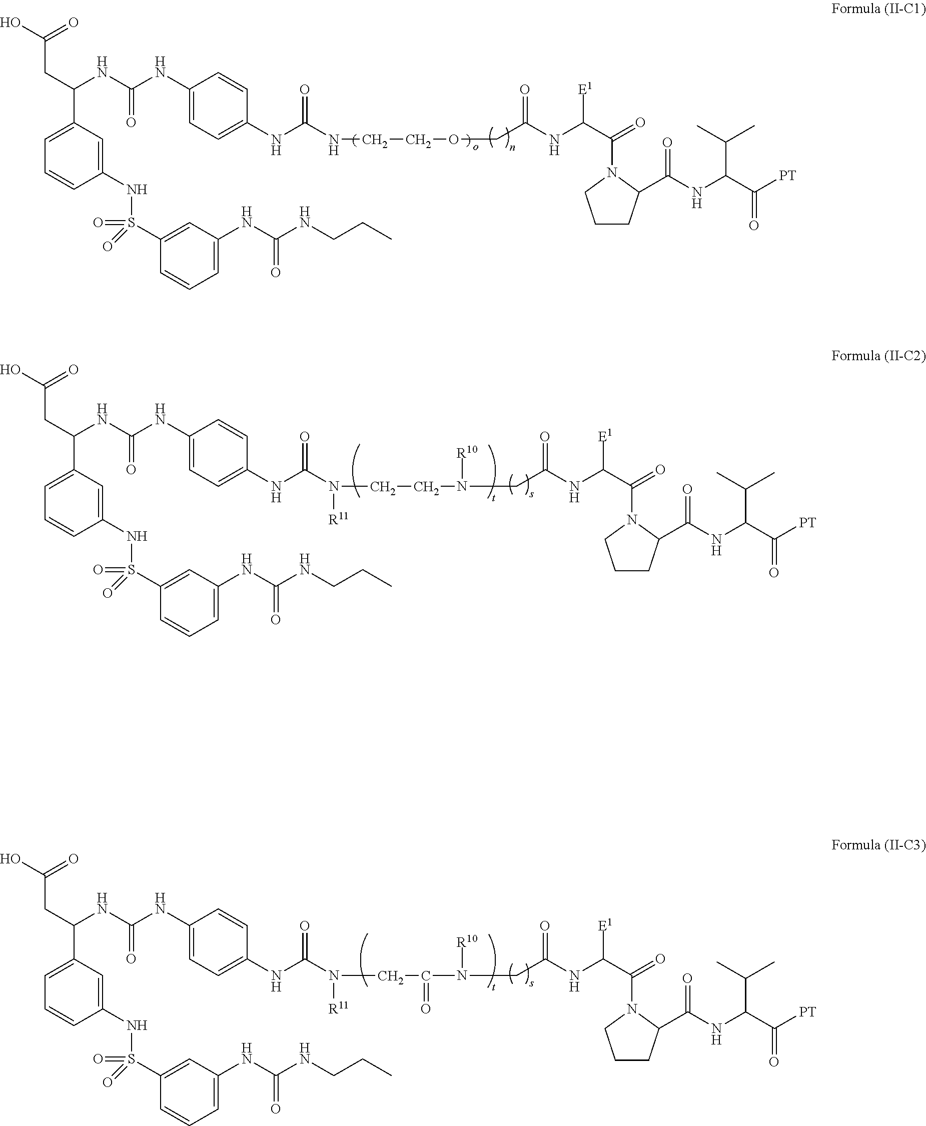

wherein:
   R¹⁰ is hydrogen or —CH₃;
   R¹¹ is hydrogen or —CH₃;
   n is 2 to 4;
   is 1 to 8;
   s is 1 to 4;
   t is 1 to 8;
   E¹ is —H, —CH₂C(═O)OH, —CH₂C(═O)NH₂, or —CH₂C(═O)OR¹; and
   R¹ is —C₁₋₆alkyl-NH₂, —C₁₋₆alkyl-N(CH₃)₂, —C₁₋₆alkyl-N(CH₃)₃⁺, or —C₁₋₆alkyl-NH—C(O)—IN.

In some embodiments, provided herein is a compound, or a pharmaceutically acceptable salt thereof; or a stereoisomer or mixture of stereoisomers thereof, having a structure of Formula (II-C1) or Formula (II-C2):
wherein:
   R¹⁰ is —CH₃;
   R¹¹ is hydrogen or —CH₃; and
E¹ is —H, —CH₂C(═O)OH, or —CH₂C(═O)NH₂.

In some embodiments, E¹ is —CH₂C(═O)OH or —CH₂C(═O)NH₂.

In some embodiments, provided herein is a compound, or a pharmaceutically acceptable salt thereof; or a stereoisomer or mixture of stereoisomers thereof, having a structure of Formula (II-A):

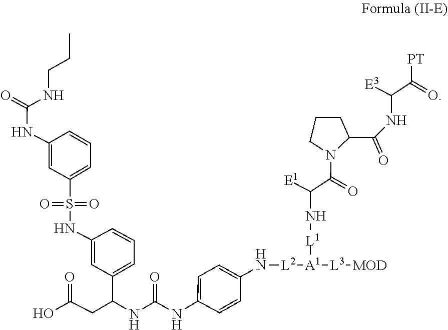

In some embodiments, provided herein is a compound, or a pharmaceutically acceptable salt thereof; or a stereoisomer or mixture of stereoisomers thereof, having a structure of Formula (II-A), wherein:
PT is a monovalent radical of a PTEFb inhibitor;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
E³ is —CH₃, —CH(CH₃)₂, —CH₂CH(CH₃)₂, or —CH(CH₃)CH₂CH₃;
A¹ is a trivalent radical (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl, heteroalkyl-heteroaryl));
each of L¹, L², and L³ is independently a substituted or unsubstituted C₁₋₃₀ alkyl, or substituted or unsubstituted heteroalkyl;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
   wherein:
   L⁶ is a substituted C₁₋₃₀ alkyl, or substituted or unsubstituted heteroalkyl;
   IN is a monovalent radical of an integrin binder; and
   MOD is a physicochemical or pharmacokinetic modulator.

In some embodiments, MOD is —COOH, —COONa —COOCH₃, —NH₂, —N(CH₃)₂, —N(CH₃)₃, —NC(═NH)NH₂, —OH, or —OCH₃. In some embodiments, MOD is —COOH, —NH₂, —N(CH₃)₂, —N(CH₃)₃⁺, —N(═NH)NH₂, or —OH. In some embodiments, MOD is an anion-forming physicochemical or pharmacokinetic modulator. In some embodiments, MOD is —COOH or —OH (i.e., —COO⁻ or —O⁻, or a salt thereof). In some embodiments, MOD is —COOH, or a salt thereof. In some embodiments, MOD is —OH, or a salt thereof. In some embodiments, MOD is a cation-forming physicochemical modulator. In some embodiments, MOD is —NH₂, —N(CH₃)₂, or —N(CH₃)₃⁺. In some embodiments, MOD is a physicochemical modulator comprising a polar amino acid, or a derivative thereof. In some embodiments, MOD is or comprises a polyamine or polyamide. In some embodiments, MOD is or comprises a polypeptide (e.g., a natural polypeptide or an unnatural polypeptide).

In some embodiments, provided herein is a compound, or a pharmaceutically acceptable salt thereof; or a stereoisomer or mixture of stereoisomers thereof, having a structure of Formula (II-E), wherein:
E¹ is hydrogen, —CH₂C(O)NH₂ or —CH₂C(O)OH;
E³ is —CH₃ or —CH(CH₃)₂;
L¹ is —C(O)—C₂₋₄ alkyl-[O—C₂₋₄ alkyl]₂₋₄-NH*,
   —C(O)—C₁₋₄ alkyl-[N(CH₃)—C₂₋₄ alkyl]₂₋₄-NH—*,
   —C(O)—C₁₋₄ alkyl-[N(CH₃)—C₂₋₄ alkyl]₂₋₄-N(CH₃)—*;
A¹ is *C(O)—CH(NH**)(C₁₋₄ alkyl-NH***), or
   *C(O)—C₁₋₄ alkyl-C(O)NH—CH(C₁₋₄ alkyl-C(O)NH—C₂₋₄ alkyl-NH**)(C(O)NH—C₂₋₄ alkyl-NH***);
L² is **C(O)—;
   **C(O)—C₂₋₄ alkyl-NHC(O)—,
   **C(O)—C₂₋₄ alkyl-[O—C₂₋₆ alkyl]₂₋₄-NHC(O)—,
   **C(O)—C₁₋₄ alkyl-[N(CH₃)—C₂₋₆ alkyl]₂₋₄-NHC(O)—, or
   **C(O)—C₁₋₄ alkyl-[N(CH₃)—C₂₋₆ alkyl]₂₋₄-N(CH₃)C(O)—;
L³ is ***C(O)—,
   ***C(O)—C₂₋₄ alkyl-NH—,
   ***C(O)—C₂₋₄ alkyl-[O—C₂₋₆ alkyl]₂₋₄-NH—,
   ***C(O)—C₁₋₄ alkyl-[N(CH₃)—C₂₋₆ alkyl]₂₋₄-NH—, or
   ***C(O)—C₁₋₄ alkyl-[N(CH₃)—C₂₋₆ alkyl]₂₋₄-N(CH₃)—; and
MOD is —COOH.

In some embodiments, A¹ is an amino acid or a derivative thereof. In some embodiments, A¹ is an amino acid, or A¹ is an amino acid derivative comprising an amino acid, wherein the carboxylate group(s) is/are substituted with heteroalkyl groups. In some embodiments, A¹ is an amino acid derivative comprising an amino acid, wherein the carboxylate group(s) is/are substituted with C₁₋₆ aminoalkyl groups. In some embodiments, A¹ is an amino acid, or A¹ is an amino acid derivative comprising an amino acid, wherein the carboxylate group(s) is/are substituted with C₁₋₆ aminoalkyl groups. In some embodiments, A¹ is an amino acid derivative comprising an amino acid (e.g., Glu or Asp), wherein the carboxylate groups are substituted with C₁₋₆ aminoalkyl groups (e.g., beta-alanine groups). In some embodiments, A¹ is an amino acid. In some embodiments, A¹ is lysine. In some embodiments, A¹ is L-Lys. In some embodiments, A¹ is D-Lys. In some embodiments, A¹ is Glu, or an aminoalkyl derivative thereof. In some embodiments, A¹ is L-Glu or a beta-alanine-substituted derivative thereof. In some embodiments, A¹ is D-Glu or a beta-alanine-substituted derivative thereof.

In some embodiments, A¹ is *C(O)—CH(NH**)(C₁₋₄ alkyl-NH***), wherein * is a bond between L¹ and A¹; ** is a bond between A¹ and L²; and *** is a bond between A¹ and L³. In some embodiments, A¹ is

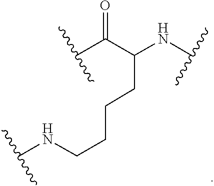 [SMILES: *NCCCCC(N*)C(*)=O]

In some embodiments, A¹ is

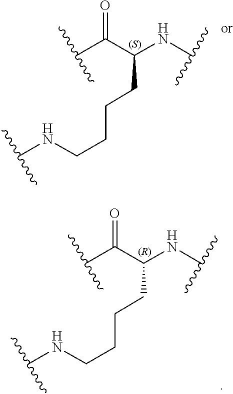

In some embodiments, A¹ is

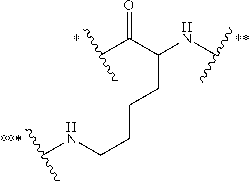 [SMILES: *NCCCCC(N*)C(*)=O]

wherein * is a bond between L¹ and A¹; ** is a bond between A¹ and L²; and *** is a bond between A¹ and L³. In some embodiments, A¹ is

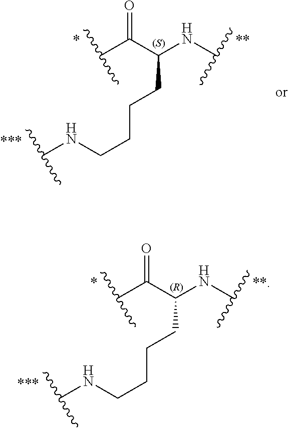

In some embodiments, L¹ is selected from:

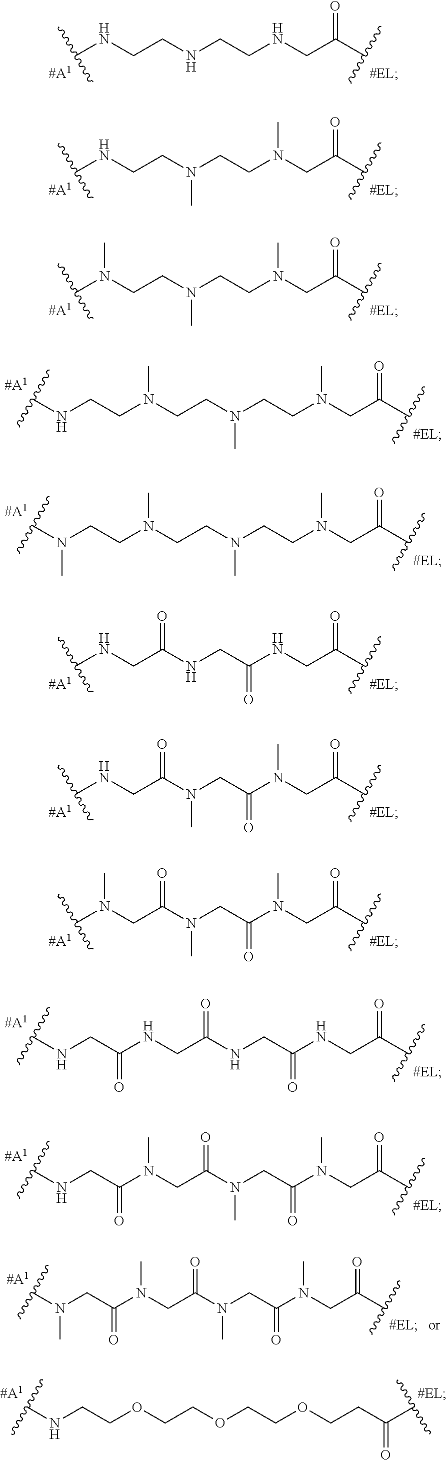

wherein #EL denotes a bond to EL; and #A¹ denotes a bond to A¹.

In some embodiments, L² and L³ are each independently selected from:

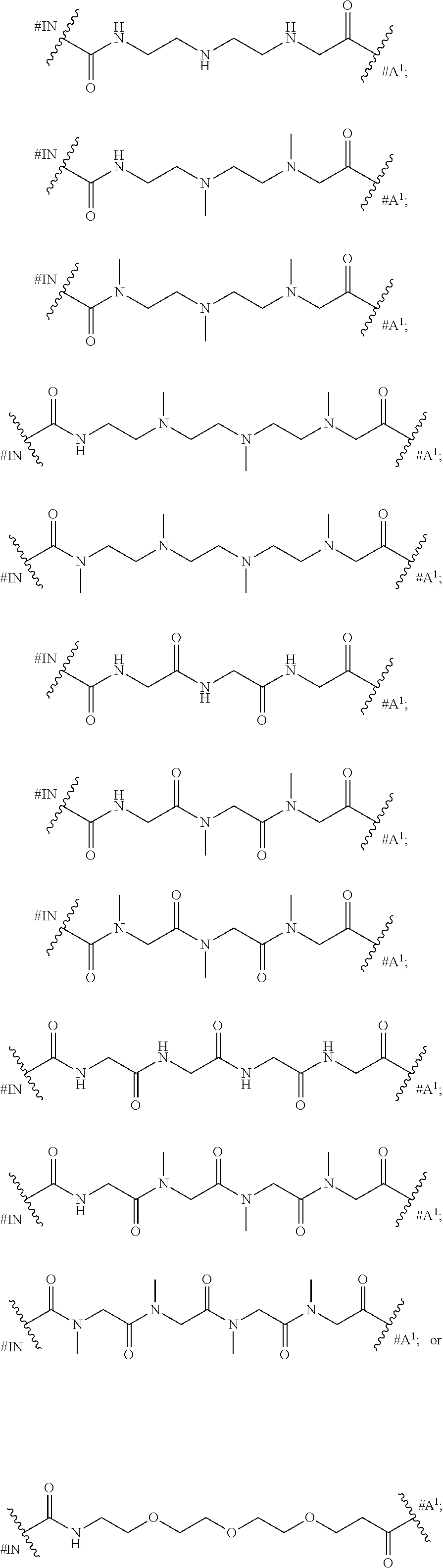

wherein #IN denotes a bond to IN or MOD; and #^{A} denotes a bond to A¹.

In some embodiments, PT is a radical of a PTEFb inhibitor. In some embodiments, PT is a radical of a macrocyclic or polycyclic small molecule PTEFb inhibitor. In some embodiments, PT is a radical of a polycyclic small molecule PTEFb inhibitor. In some embodiments, PT is a radical of a macrocyclic small molecule PTEFb inhibitor.

In some embodiments, PT is a radical of a sulfoximine-linked PTEFb inhibitor. In some embodiments, PT is a radical of a sulfoximine-linked macrocyclic or polycyclic small molecule PTEFb inhibitor. In some embodiments, PT is a radical of a sulfoximine-linked polycyclic small molecule PTEFb inhibitor. In some embodiments, PT is a radical of a sulfoximine-linked macrocyclic small molecule PTEFb inhibitor.

In some embodiments, PT is a radical of a PTEFb inhibitor having a structure represented by the formula:

—(SFX)-(L^{A})-(Ring A)-(L^{B})-(Ring B)-(L^{C})-(Ring C)-(L^{D})-; (iv)
wherein:
SFX is a sulfoximine moiety (e.g., —N═S(═O)(C₁₋₆alkyl)-)
L^{A} is a bond or C₁₋₆alkyl;
Ring A is an optionally substituted carbocycle or optionally substituted heterocycle (e.g., optionally substituted phenyl or optionally substituted heteroaryl);
L^{B} is a bond, optionally substituted C₁₋₆alkyl, or optionally substituted heteroalkyl (e.g., —CH₂—, —C(═O)NH— —NH—, —N(CH₃)—, —NHC(═O)—, —O—, or —S—;);
Ring B is an optionally substituted carbocycle or optionally substituted heterocycle (e.g., optionally substituted phenyl or optionally substituted heteroaryl);
L^{C} is a bond, optionally substituted C₁₋₆alkyl, or optionally substituted heteroalkyl (e.g., —CH₂—, —C(═O)NH— —NH—, —N(CH₃)—, —NHC(═O)—, —O—, or —S—;);
Ring C is an optionally substituted carbocycle or optionally substituted heterocycle (e.g., optionally substituted phenyl or optionally substituted heteroaryl); and
L^{D} is absent; or an optionally substituted heteroalkyl bonded to Ring A, thereby forming an optionally substituted macrocyclic Ring D (e.g., a ring comprising 12 to 20 atoms selected from C, N, O, and S).

In some embodiments, PT has a structure represented by the formula:

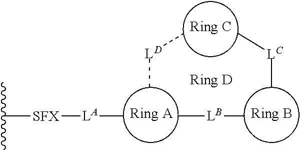

wherein if LD is present, it connects Ring C and Ring A to form a macrocyclic Ring D.

In some embodiments, LD is absent. In some embodiments, LD is an optionally substituted heteroalkyl group. In some embodiments, LD is a heteroalkyl group comprising 2 to 12 atoms selected from C, N, O, and S. In some embodiments, LD is a heteroalkyl group of the formula —O—(C₁₋₆alkyl)-O—, —NH—(C₁₋₆alkyl)-O—, or —NH—(C₁₋₆alkyl)-NH—. In some embodiments, LD is a heteroalkyl group of the formula —O—(C₁₋₆alkyl)-O—. In some embodiments, L^{C} is a bond. In some embodiments, L^{B} is —CH₂—, —C(═O)NH— —NH—, —N(CH₃)—, —NHC(═O)—, —O—, or —S—. In some embodiments, L^{B} is —CH₂—, —NH—, —O—, or —S—. In some embodiments, L^{B} is —CH₂—. In some embodiments, L^{B} is —NH—. In some embodiments, L^{B} is —O—. In some embodiments, L^{A} is C₁₋₆ alkyl. In some embodiments, L^{A} is —CH₂.

In some embodiments, PT is a radical of a PTEFb inhibitor having a structure represented by the formula:

—(SFX)-(L^{A})-(Ring A)-(L^{B})-(Ring B)-(L^{C})-(Ring C)-(L^{D})-; (iv)
wherein:
SFX is a sulfoximine moiety (e.g., —N═S(═O)(C₁₋₆alkyl)-)
L^{A} is —CH₂—;
L^{B} is —CH₂—, —C(═O)NH— —NH—, —N(CH₃)—, —NHC(═O)—, —O—, or —S—;
L^{C} is a bond; and
L^{D} is absent.

In some embodiments, PT is a radical of a PTEFb inhibitor having a structure represented by the formula:

—(SFX)-(L^{A})-(Ring A)-(L^{B})-(Ring B)-(L^{C})-(Ring C)-(L^{D})-; (iv)
wherein:
SFX is a sulfoximine moiety (e.g., —N═S(═O)(C₁₋₆alkyl)-)
L^{A} is —CH₂—;
L^{B} is —CH₂—, —C(═O)NH— —NH—, —N(CH₃)—, —NHC(═O)—, —O—, or —S—;
L^{C} is a bond; and
L^{D} is a linker having the formula —O—(C₁₋₆alkyl)-O— or —O—(C₁₋₆alkyl)-NH—, linking Ring C to Ring A, thereby forming an optionally substituted macrocyclic Ring D.

In some embodiments, PT has a structure represented by the formula:

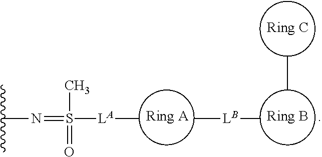

In some embodiments, PT has a structure represented by the formula:

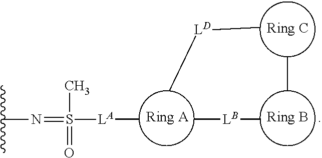

In some embodiments, L^{B} is —CH₂—, —C(═O)NH— —NH—, —N(CH₃)—, —NHC(═O)—, —O—, or —S—. In some embodiments, L^{B} is —O—, —NH—, —N(CH₃)—, —CH₂—, or —S—. In some embodiments, L^{B} is —NH—. In some embodiments, L^{A} is C₁₋₆ alkyl. In some embodiments, L^{A} is methylene, ethylene, or propylene. In some embodiments, L^{A} is —CH₂—. In some embodiments, LD is a linker having the formula —O—(C₁₋₆alkyl)-O— or —O—(C₁₋₆alkyl)-NH—. In some embodiments, LD is a linker having the formula —O—(C₁₋₁₀alkyl)-O— or —O—(C₁₋₁₀alkyl)-NH—. In some embodiments, LD is a linker having the formula —O-(heteroalkyl)-O— or —O-(heteroalkyl)-NH—.

In some embodiments, Ring A is a carbocycle or heterocycle. In some embodiments, Ring A is a six-membered carbocycle or heterocycle. In some embodiments, Ring A is an optionally substituted phenyl or optionally substituted six-membered heteroaryl (e.g., pyridine, pyrazine, pyridazine, pyrimidine, triazine, or tetrazine). In some embodiments, Ring A is an optionally substituted phenyl or an optionally substituted pyridine.

In some embodiments, Ring B is a carbocycle or heterocycle. In some embodiments, Ring B is a six-membered carbocycle or heterocycle. In some embodiments, Ring B is an optionally substituted phenyl or optionally substituted six-membered heteroaryl (e.g., pyridine, pyrazine, pyridazine, pyrimidine, triazine, or tetrazine). In some embodiments, Ring B is an optionally substituted phenyl or an optionally substituted pyridine. In some embodiments, Ring B is an optionally substituted six-membered heteroaryl. In some embodiments, Ring B is an optionally substituted pyridine, pyrimidine, or triazine.

In some embodiments, Ring C is a carbocycle or heterocycle. In some embodiments, Ring C is a six-membered carbocycle or heterocycle. In some embodiments, Ring C is an optionally substituted phenyl or optionally substituted six-membered heteroaryl (e.g., pyridine, pyrazine, pyridazine, pyrimidine, triazine, or tetrazine). In some embodiments, Ring C is an optionally substituted phenyl or an optionally substituted pyridine.

In some embodiments, PT is a radical of a PTEFb inhibitor having a structure represented by the formula:

—(SFX)-(L^{A})-(Ring A)-(L^{B})-(Ring B)-(L^{C})-(Ring C)-(L^{D})-; (iv)
wherein:
SFX is a sulfoximine moiety (e.g., —N═S(═O)(C₁₋₆alkyl)-)
L^{A} is —CH₂—;
Ring A is optionally substituted phenyl or optionally substituted pyridine;
L^{B} —CH₂—, —C(═O)NH— —NH—, —N(CH₃)—, —NHC(═O)—, —O—, or —S—;
Ring B is an optionally substituted pyridine, optionally substituted pyrimidine, or optionally substituted triazine;
L^{C} is a bond;
Ring C is an optionally substituted phenyl; and
L^{D} is absent, or a linker having the formula —O—(C₁₋₆alkyl)-O—, or —O—(C₁₋₆alkyl)-NH—, wherein the linker conjoins Ring C and Ring A, forming a macrocyclic Ring D.

In some embodiments, provided herein is a compound, or a pharmaceutically acceptable salt thereof; or a stereoisomer or mixture of stereoisomers thereof, having a structure of Formula (III-A), Formula (III-C), or Formula (III-E):

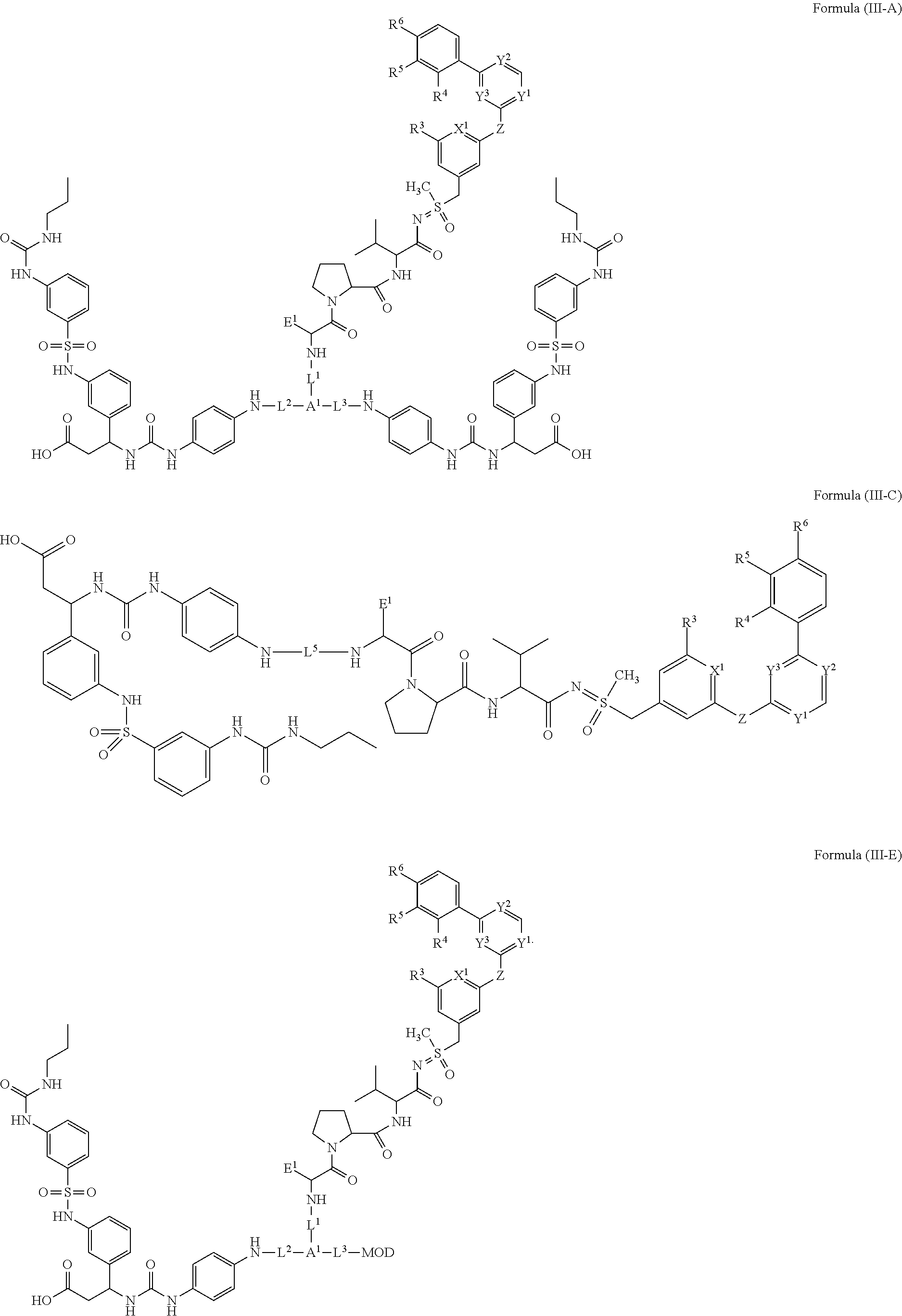

In some embodiments, each of L¹, L², L³, L⁵, and L⁶, is independently a simple spacer (defined herein) or a polymeric spacer (defined herein).

In some embodiments, each of L¹, L², and L³ is a simple spacer (defined herein) or a polymeric spacer having a structure represented by formula (i), (ii), or (iii) below:

—(CO)ₘ(CH₂)ₙ(OC₂₋₆ alkyl)ₒ(NH)ₚ(CO)_{q}—; (i)

—(CO)ᵣ(CH₂)ₛ(NR¹⁰C₂₋₆ alkyl)ₜ(NR¹¹)ᵤ(CO)ᵥ—; or (ii)

—(CO)ᵣ(CH₂)ₛ(NR¹⁰C(O)—C₁₋₆ alkyl)ₜ(NR¹¹)ᵤ(CO)ᵥ—; (iii)
wherein:
R¹⁰ is, in each instance, independently selected from hydrogen or C₁₋₃ alkyl;
R¹¹ is, in each instance, independently selected from hydrogen or C₁₋₃ alkyl;
m is 0 or 1;
n is 0 to 10;
o is 1 to 10;
p is 0 or 1;
q is 0 or 1;
r is 0 or 1;
s is 0 to 10;
t is 1 to 10;
u is 0 or 1;
v is 0 or 1.

In some embodiments, provided herein is a compound, or a pharmaceutically acceptable salt thereof; or a stereoisomer or mixture of stereoisomers thereof, having a structure of Formula (III-A), Formula (III-C) or wherein:
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl;
or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl;
or R³ and R⁵ are taken together to form a bivalent radical of the formula —O—C₂₋₁₀ alkyl-O—, —NH—C₂₋₁₀ alkyl-O—, or —NH—C₂₋₁₀ alkyl-NH—; and
R⁶ is hydrogen, halogen, —OH, or —O—C₁₋₄alkyl.

In some embodiments, provided herein is a compound, or a pharmaceutically acceptable salt thereof; or a stereoisomer or mixture of stereoisomers thereof, having a structure of Formula (III-A):

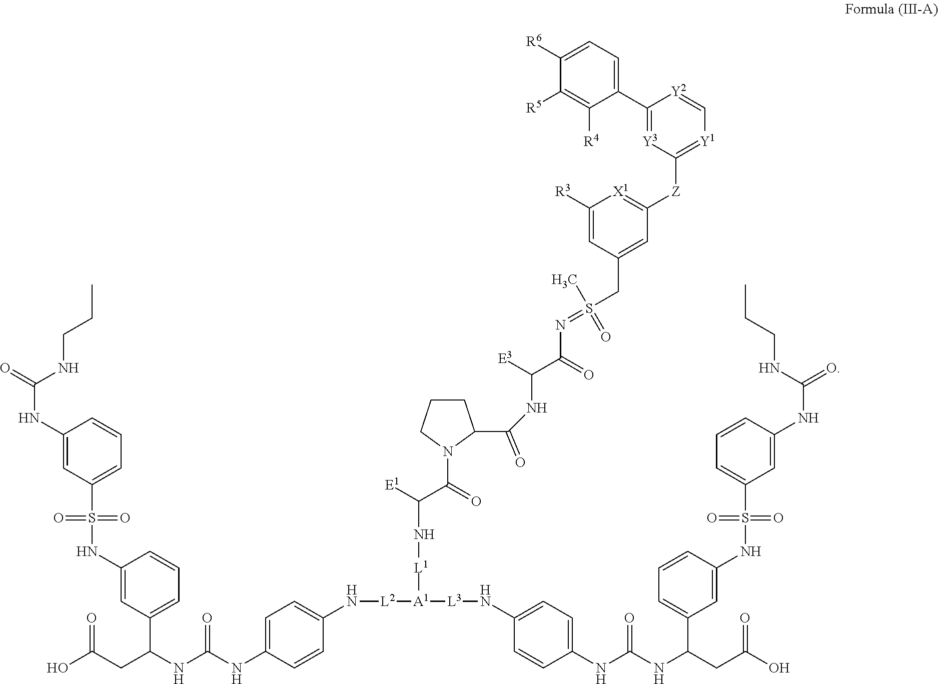

In some embodiments, provided herein is a compound, or a pharmaceutically acceptable salt thereof; or a stereoisomer or mixture of stereoisomers thereof, having a structure of Formula (III-A), wherein:
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl;
or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl;
or R³ and R⁵ are taken together to form a bivalent radical of the formula —O—C₂₋₁₀ alkyl-O—, —NH—C₂₋₁₀ alkyl-O—, or —NH—C₂₋₁₀ alkyl-NH—;
R⁶ is hydrogen, halogen, —OH, or —O—C₁₋₄alkyl;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
E³ is —CH₃, —CH(CH₃)₂, —CH₂CH(CH₃)₂, or —CH(CH₃)CH₂CH₃;
A¹ is a trivalent radical (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl));
each of L¹, L², and L³ is independently a substituted or unsubstituted C₁₋₃₀ alkyl, or substituted or unsubstituted heteroalkyl;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
   wherein:
   L⁶ is a substituted C₁₋₃₀ alkyl, or substituted or unsubstituted heteroalkyl; and
   IN is a monovalent radical of an integrin binder.

In some embodiments, provided herein is a compound, or a pharmaceutically acceptable salt thereof; or a stereoisomer or mixture of stereoisomers thereof, having a structure of Formula (III-A), wherein:
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl;
or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl;
or R³ and R⁵ are taken together to form a bivalent radical of the formula —O—C₂₋₁₀ alkyl-O—, —NH—C₂₋₁₀ alkyl-O—, or —NH—C₂₋₁₀ alkyl-NH—;
R⁶ is hydrogen, halogen, —OH, or —O—C₁₋₄alkyl;
E¹ is hydrogen, —CH₂C(O)NH₂ or —CH₂C(O)OH;
L¹ is —C(O)—C₂₋₄ alkyl-[O—C₂₋₄ alkyl]₂₋₄-NH*,
   —C(O)—C₁₋₄ alkyl-[N(CH₃)—C₂₋₄ alkyl]₂₋₄-NH—*,
   —C(O)—C₁₋₄ alkyl-[N(CH₃)—C₂₋₄ alkyl]₂₋₄-N(CH₃)—*;
A¹ is *C(O)—CH(NH**)(C₁₋₄ alkyl-NH***), or
   *C(O)—C₁₋₄ alkyl-C(O)NH—CH(C₁₋₄ alkyl-C(O)NH—C₂₋₄ alkyl-NH**)(C(O)NH—C₂₋₄ alkyl-NH***);
L² is **C(O)—;
   **C(O)—C₂₋₄ alkyl-NHC(O)—,
   **C(O)—C₂₋₄ alkyl-[O—C₂₋₆ alkyl]₂₋₄-NHC(O)—,
   **C(O)—C₁₋₄ alkyl-[N(CH₃)—C₂₋₆ alkyl]₂₋₄-NHC(O)—, or
   **C(O)—C₁₋₄ alkyl-[N(CH₃)—C₂₋₆ alkyl]₂₋₄-N(CH₃)C(O)—; and
L³ is ***C(O)—
   ***(C(O)—C₂₋₄ alkyl-NHC(O)—,
   ***C(O)—C₂₋₄ alkyl-[O—C₂₋₆ alkyl]₂₋₄-NHC(O)—,
   ***C(O)—C₁₋₄ alkyl-[N(CH₃)—C₂₋₆ alkyl]₂₋₄-NHC(O)—, or
   ***C(O)—C₁₋₄ alkyl-[N(CH₃)—C₂₋₆ alkyl]₂₋₄-N(CH₃)C(O)—.

In some embodiments, provided herein is a compound, or a pharmaceutically acceptable salt thereof; or a stereoisomer or mixture of stereoisomers thereof, having a structure of Formula (III-A1):

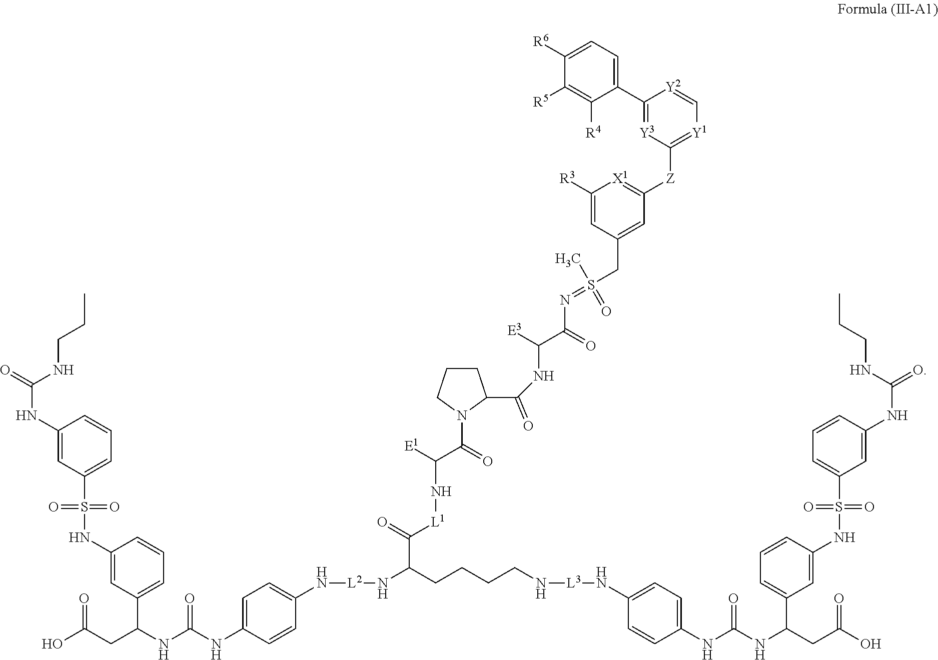

In some embodiments, provided herein is a compound, or a pharmaceutically acceptable salt thereof; or a stereoisomer or mixture of stereoisomers thereof, having a structure of Formula (III-A2):

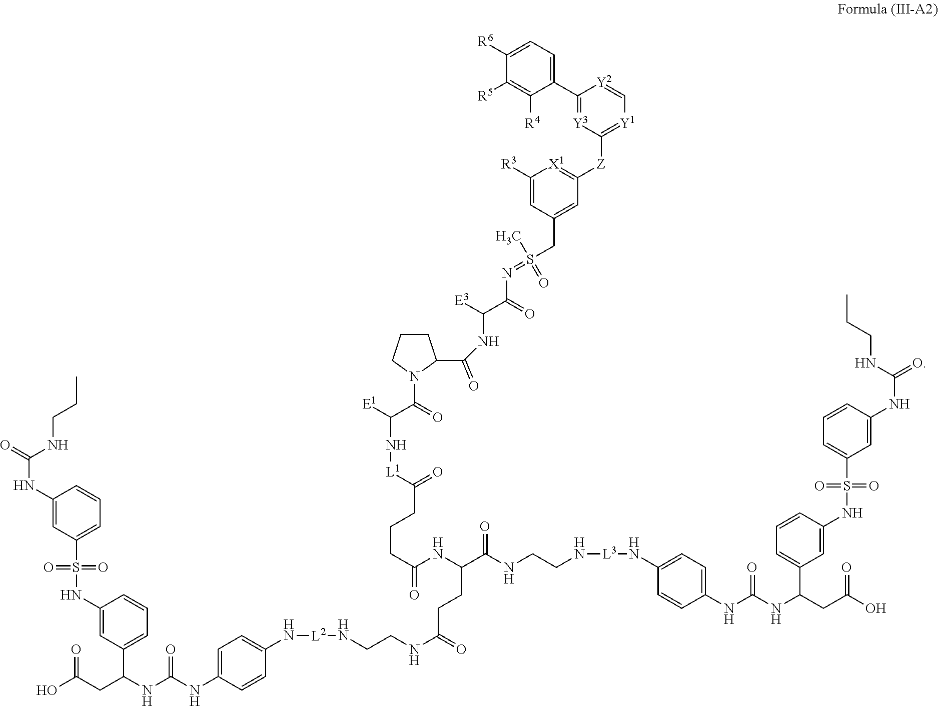

In some embodiments,
E¹ is hydrogen, —CH₂C(O)OH, or —CH₂C(O)NH₂;
L¹ is —C(O)—C₂₋₄ alkyl-[O—C₂₋₆ alkyl]₁₋₈-NH; or
   —C(O)—C₁₋₄ alkyl-[N(CH₃)—C₁₋₆ alkyl]₁₋₈-NH—,
L² is —C(O)—C₂₋₄ alkyl-[O—C₂₋₆ alkyl]₁₋₈-NHC(O); or
   —C(O)—C₁₋₄ alkyl-[N(CH₃)—C₁₋₆ alkyl]₁₋₈-NHC(O)—, and
L³ is —C(O)—C₂₋₄ alkyl-[O—C₂₋₆ alkyl]₁₋₈-NHC(O); or
   —C(O)—C₁₋₄ alkyl-[N(CH₃)—C₁₋₆ alkyl]₁₋₈-NHC(O)—.

In some embodiments,
E¹ is hydrogen, —CH₂C(O)OH, or —CH₂C(O)NH₂;
L¹ is —C(O)—C₂₋₄ alkyl-[O—C₂₋₄ alkyl]₂₋₄-NH; or
   —C(O)—C₁₋₄ alkyl-[N(CH₃)—C₁₋₄ alkyl]₂₋₄-NH—,
L² is —C(O)—C₂₋₄ alkyl-[O—C₂₋₄ alkyl]₂₋₄-NHC(O); or
   —C(O)—C₁₋₄ alkyl-[N(CH₃)—C₂₋₄ alkyl]₂₋₄-NHC(O)—, and
L³ is —C(O)—C₂₋₄ alkyl-[O—C₂₋₄ alkyl]₂₋₄-NHC(O); or
   —C(O)—C₁₋₄ alkyl-[N(CH₃)—C₂₋₄ alkyl]₂₋₄-NHC(O)—.

In some embodiments,
E¹ is hydrogen;
L¹ is —C(O)—CH₂—[N(CH₃)CH₂CH₂]₃—NH*;
L² is **C(O)—CH₂CH₂—[OCH₂CH₂]₃—NHC(O)—; and
L³ is ***C(O)—CH₂CH₂—[OCH₂CH₂]₃—NHC(O)—.

In some embodiments,
E¹ is —CH₂C(O)NH₂;
L¹ is —C(O)—CH₂—[N(CH₃)CH₂CH₂]₃—NH*;
L² is **C(O)—CH₂CH₂—[OCH₂CH₂]₃—NHC(O)—; and
L³ is ***C(O)—CH₂CH₂—[OCH₂CH₂]₃—NHC(O)—.

In some embodiments,
E¹ is —CH₂C(O)OH;
L¹ is —C(O)—CH₂CH₂—[OCH₂CH₂]₃—NH*;
L² is **C(O)—CH₂CH₂—[OCH₂CH₂]₃—NHC(O)—; and
L³ is ***C(O)—CH₂CH₂—[OCH₂CH₂]₃—NHC(O)—.

In some embodiments,
X¹ is CH or N;
Y¹ is CH or N;
Y² is CF or N;
Y³ is CH or N;
Z is —NH—;
R³ is hydrogen; and
R⁴ is —OCH₃; or
R³ and R⁴ are taken together to form —O—C₂₋₁₀ alkyl-O— or —NH—C₂₋₁₀ alkyl-O—; and
R⁵ is hydrogen, halogen, —OH, or —O—C₁₋₄alkyl; or
R³ and R⁵ are taken together to form a bivalent radical of the formula —O—C₂₋₁₀ alkyl-O—, —NH—C₂₋₁₀ alkyl-O—, or —NH—C₂₋₁₀ alkyl-NH—; and
R⁶ is halogen.

In some embodiments,
X¹ is CH or N;
Y² is CF or N;
Y³ is CH or N;
R³ is hydrogen;
R⁴ is —OCH₃; or
R³ and R⁴ are taken together to form —O—C₂₋₁₀ alkyl-O—;
R⁵ is hydrogen; or
R³ and R⁵ are taken together to form —O—C₂₋₁₀ alkyl-NH—;
E¹ is —CH₂C(O)OH or —CH₂C(O)NH₂;
L¹ is —C(O)—CH₂CH₂—[OCH₂CH₂]₃—NH— or —C(O)—CH₂—[N(CH₃)CH₂CH₂]₃—NH—;
L² is —C(O)—CH₂CH₂—[OCH₂CH₂]₃—NHC(O)—; and
L³ is —C(O)—CH₂CH₂—[OCH₂CH₂]₃—NHC(O)—.

In some embodiments, provided herein is a compound, or a pharmaceutically acceptable salt thereof; or a stereoisomer or mixture of stereoisomers thereof, having a structure of Formula (III-A3), Formula (III-A4), or Formula (III-A5):

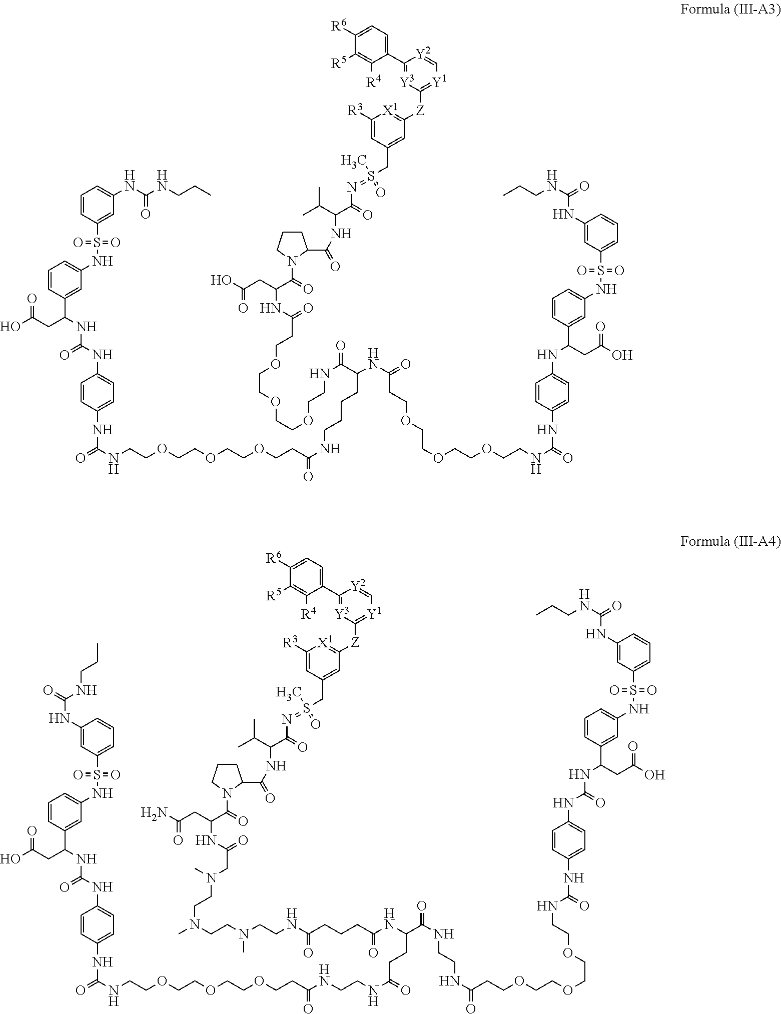

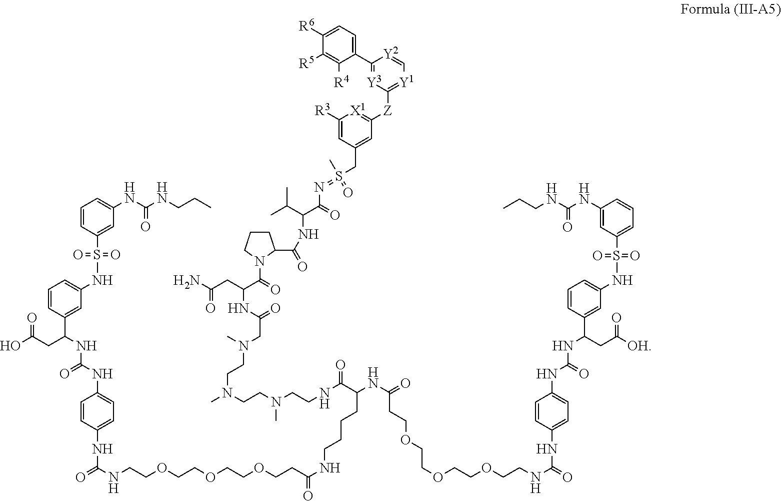

In some embodiments, provided herein is a compound that is:

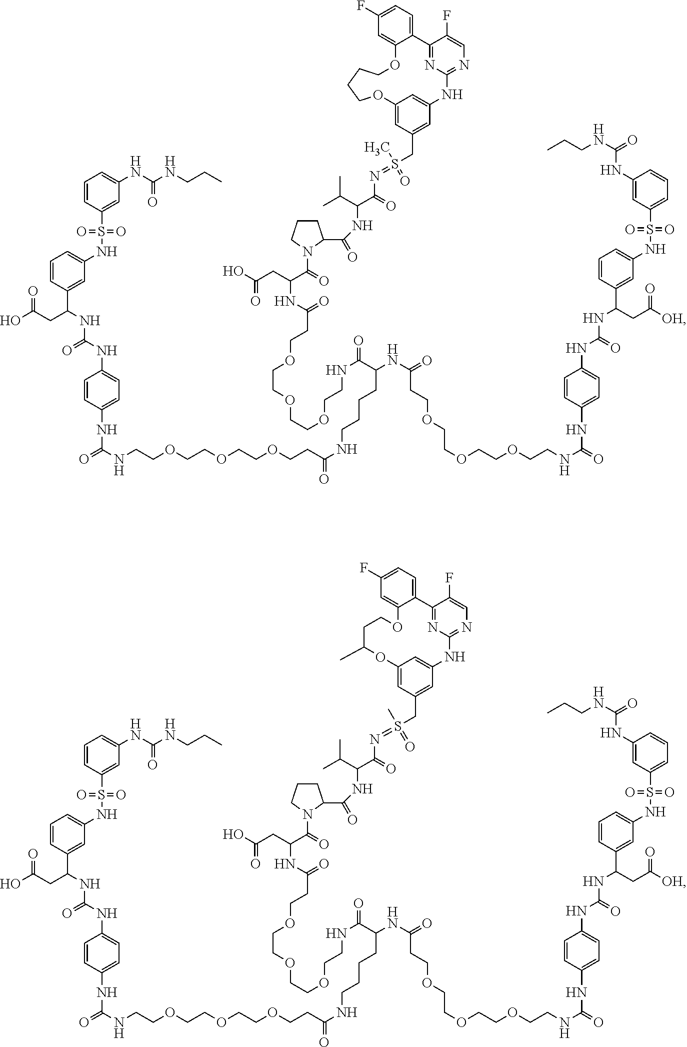

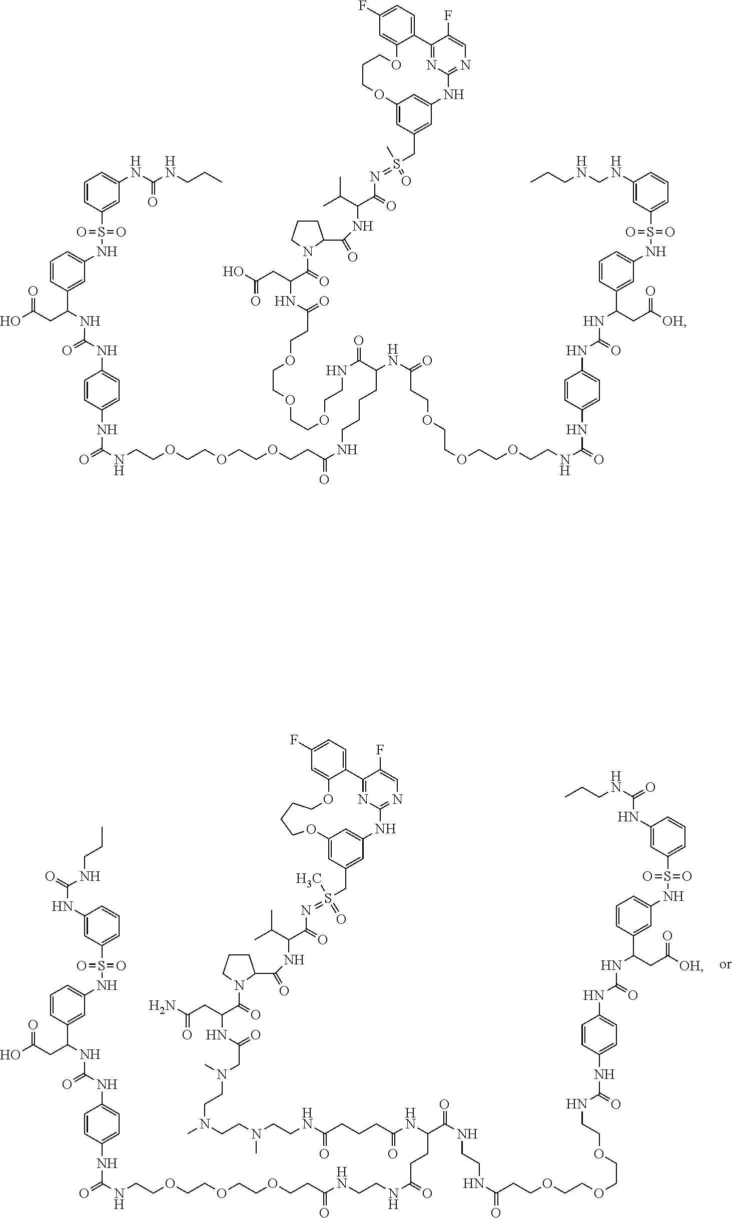

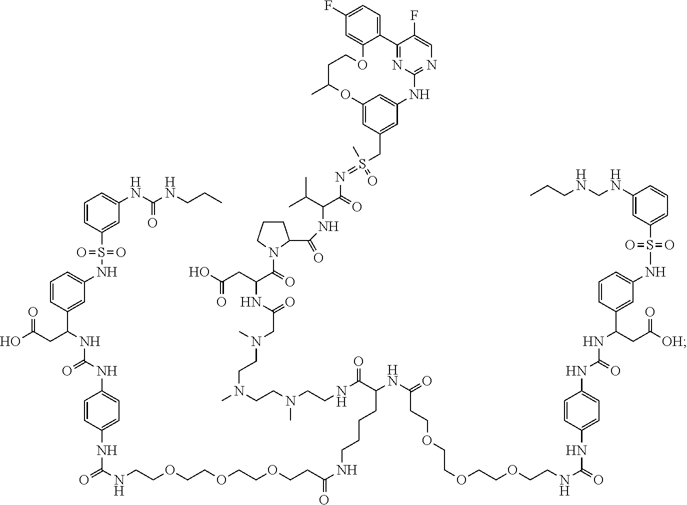 [SMILES: CCCNCNc1cccc(S(=O)(=O)Nc2cccc(C(CC(=O)O)NC(=O)Nc3ccc(NC(=O)NCCOCCOCCOCCC(=O)NC(CCCCNC(=O)CCOCCOCCOCCNC(=O)Nc4ccc(NC(=O)NC(CC(=O)O)c5cccc(NS(=O)(=O)c6cccc(NC(=O)NCCC)c6)c5)cc4)C(=O)NCCN(C)CCN(C)CCN(C)CC(=O)NC(CC(=O)O)C(=O)N4CCCC4C(=O)NC(C(=O)N=S(C)(=O)Cc4cc5cc(c4)OC(C)CCOc4cc(F)ccc4-c4nc(ncc4F)N5)C(C)C)cc3)c2)c1]

or a pharmaceutically acceptable salt thereof; or a stereoisomer or mixture of stereoisomers thereof.

In some embodiments, provided herein is a compound, or a pharmaceutically acceptable salt thereof; or a stereoisomer or mixture of stereoisomers thereof, having a structure of Formula (III-B):

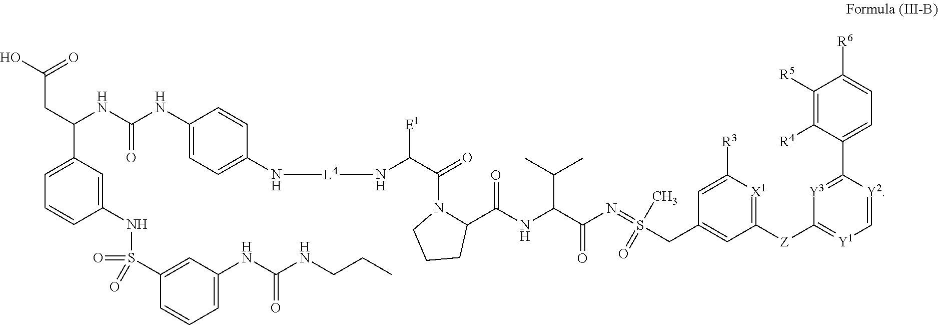

In some embodiments, provided herein is a compound, or a pharmaceutically acceptable salt thereof; or a stereoisomer or mixture of stereoisomers thereof, having a structure of Formula (III-B), wherein:
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
   wherein:
   L⁶ is a substituted C₁₋₃₀ alkyl, or substituted or unsubstituted heteroalkyl; and
   IN is a monovalent radical of an integrin binder
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl;
or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl;
or R³ and R⁵ are taken together to form a bivalent radical of the formula —O—C₂₋₁₀ alkyl-O—, —O—C₂₋₁₀ alkyl-NH—, or —NH—C₂₋₁₀ alkyl-NH—;
R⁶ is hydrogen, halogen, —OH, or —O—C₁₋₄alkyl;
L⁴ is a group of the formula:

—(CO)ᵣ—(CH₂)ₛ—(NR¹⁰—C₂₋₆ alkyl)ₜ-(NR¹¹)ᵤ—(CO)ᵥ—; or

—(CO)ᵣ—(CH₂)ₛ—(NR¹⁰—C(O)C₁₋₆ alkyl)ₜ-(NR¹¹)ᵤ—(CO)ᵥ—;
wherein:
R¹⁰ is hydrogen or C₁₋₃ alkyl;
R¹¹ is hydrogen or C₁₋₃ alkyl;
r is 0 or 1;
s is 0-10;
t is 1-10;
u is 0 or 1; and
v is 0 or 1.

In some embodiments, provided herein is a compound, or a pharmaceutically acceptable salt thereof; or a stereoisomer or mixture of stereoisomers thereof, having a structure of Formula (III-B1):

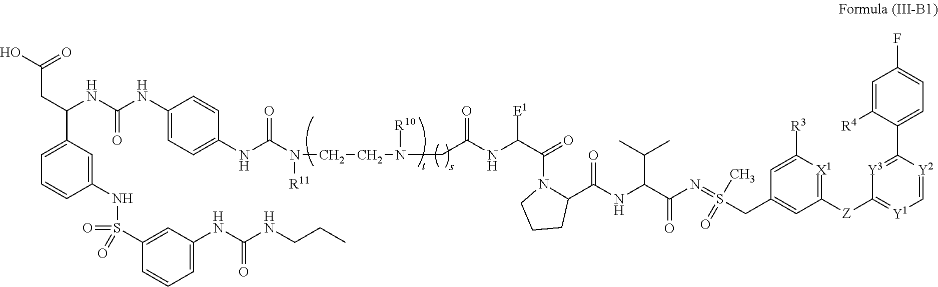

In some embodiments, provided herein is a compound of Formula (II-B1), or a pharmaceutically acceptable salt thereof; or a stereoisomer or mixture of stereoisomers thereof, wherein:
E¹ is hydrogen, —CH₂C(O)NH₂, —CH₂C(O)OH, —CH₂C(O)OR¹, —CH₂CH₂C(O)OH or —CH₂CH₂C(O)OR¹;
   R¹ is C₁₋₆alkyl substituted with —NH₂ or, —NHL⁶-IN, —N(CH₃)₂, or —N(CH₃)₃⁺;
   wherein:
   L⁶ is substituted C₁₋₃₀ alkyl, or substituted or unsubstituted heteroalkyl; and
   IN is a monovalent radical of an integrin binder;
R¹⁰ is hydrogen or —CH₃;
R¹¹ is hydrogen or —CH₃;
s is 1 to 4; and
t is 1 to 8.

In some embodiments, provided herein is a compound, or a pharmaceutically acceptable salt thereof; or a stereoisomer or mixture of stereoisomers thereof, having a structure of Formula (III-B1), wherein:
E¹ is hydrogen, —CH₂C(O)OH, or —CH₂C(O)NH₂;
R¹⁰ is —CH₃;
R¹¹ is hydrogen or —CH₃;
s is 1 to 4; and
t is 2 to 4.

In some embodiments,
X¹ is CH or N;
Y² is CF or N;
Y³ is CH or N;
R³ is hydrogen; and
R⁴ is —OCH₃; or
R³ and R⁴ are taken together to form —O—C₂₋₁₀ alkyl-O— or —O—C₂₋₁₀ alkyl-NH—; and
R⁵ is hydrogen; or
R³ and R⁵ are taken together to form —O—C₂₋₁₀ alkyl-O— or —O—C₂₋₁₀ alkyl-NH—;

In some embodiments,
X¹ is CH or N;
Y² is CF or N;
Y³ is CH or N;
R³ is hydrogen;
R⁴ is —OCH₃; or
R³ and R⁴ are taken together to form —O—C₂₋₁₀ alkyl-O— or —O—C₂₋₁₀ alkyl-NH—;
R⁵ is hydrogen; or
R³ and R⁵ are taken together to form —O—C₂₋₁₀ alkyl-O— or —O—C₂₋₁₀ alkyl-NH—;
E¹ is —CH₂C(O)NH₂;
R¹⁰ is —CH₃;
R¹¹ is hydrogen or —CH₃;
s is 1 to 4; and
t is 2 to 4.

In some embodiments, provided herein is a compound, or a pharmaceutically acceptable salt thereof; or a stereoisomer or mixture of stereoisomers thereof, having a structure of Formula (III-C):

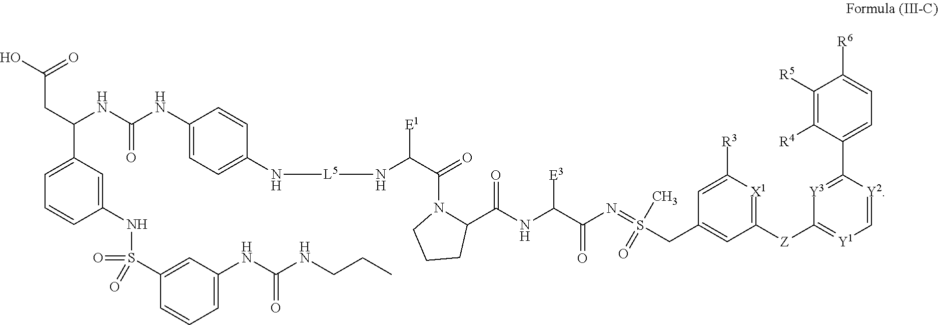

In some embodiments, provided herein is a compound, or a pharmaceutically acceptable salt thereof; or a stereoisomer or mixture of stereoisomers thereof, having a structure of Formula (III-C), wherein:
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
   wherein:
   L⁶ is a substituted C₁₋₃₀ alkyl, or substituted or unsubstituted heteroalkyl; and
   IN is a monovalent radical of an integrin binder
E³ is —CH₃, —CH(CH₃)₂, —CH₂CH(CH₃)₂, or —CH(CH₃)CH₂CH₃;
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl;
or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl;
or R³ and R⁵ are taken together to form a bivalent radical of the formula —O—C₂₋₁₀ alkyl-O—, —O—C₂₋₁₀ alkyl-NH—, or —NH—C₂₋₁₀ alkyl-NH—;
R⁶ is hydrogen, halogen, —OH, or —O—C₁₋₄alkyl; and
L⁵ is a substituted or unsubstituted alkyl, or substituted or unsubstituted heteroalkyl.

In some embodiments, provided herein is a compound, or a pharmaceutically acceptable salt thereof; or a stereoisomer or mixture of stereoisomers thereof, wherein L⁵ is a linker having a structure represented by the formula:

—(CO)ₘ(CH₂)ₙ(OC₂₋₆ alkyl)ₒ(NH)ₚ(CO)_{q}— or (i)

—(CO)ᵣ(CH₂)ₛ(NR¹⁰C₂₋₆ alkyl)ₜ(NR¹¹)ᵤ(CO)ᵥ—; (ii)
wherein:
R¹⁰ is hydrogen or C₁₋₃ alkyl;
R¹¹ is hydrogen or C₁₋₃ alkyl;
m is 0 or 1;
n is 0 to 10;
o is 1 to 10;
p is 0 or 1;
q is 0 or 1;
r is 0 or 1;
s is 0 to 10;
t is 1 to 10;
u is 0 or 1; and
v is 0 or 1.

In some embodiments, provided herein is a compound, or a pharmaceutically acceptable salt thereof; or a stereoisomer or mixture of stereoisomers thereof, wherein:
L⁵ is a linker having a structure represented by the formula:

—(CO)ₘ(CH₂)ₙ(OC₂₋₆ alkyl)ₒ(NH)ₚ(CO)_{q};
wherein:
m is 0 or 1;
n is 2 to 4;
is 1 to 8;
p is 0 or 1; and
q is 0 or 1.

In some embodiments, provided herein is a compound, or a pharmaceutically acceptable salt thereof; or a stereoisomer or mixture of stereoisomers thereof, wherein:
L⁵ is a linker having a structure represented by the formula:

—(CO)ᵣ—(CH₂)ₛ—(NR¹⁰—C₂₋₆ alkyl)ₜ-(NR¹¹)ᵤ—(CO)ᵥ—; or

—(CO)ᵣ—(CH₂)ₛ—(NR¹⁰C(O)—C₁₋₆ alkyl)ₜ-(NR¹¹)ᵤ—(CO)ᵥ—; or
wherein:
R¹⁰ is hydrogen or C₁₋₃ alkyl;
R¹¹ is hydrogen or C₁₋₃ alkyl;
r is 0 or 1;
s is 2 to 4;
t is 1 to 8;
u is 0 or 1; and
v is 0 or 1.

In some embodiments, provided herein is a compound, or a pharmaceutically acceptable salt thereof; or a stereoisomer or mixture of stereoisomers thereof, having a structure of Formula (III-C1):

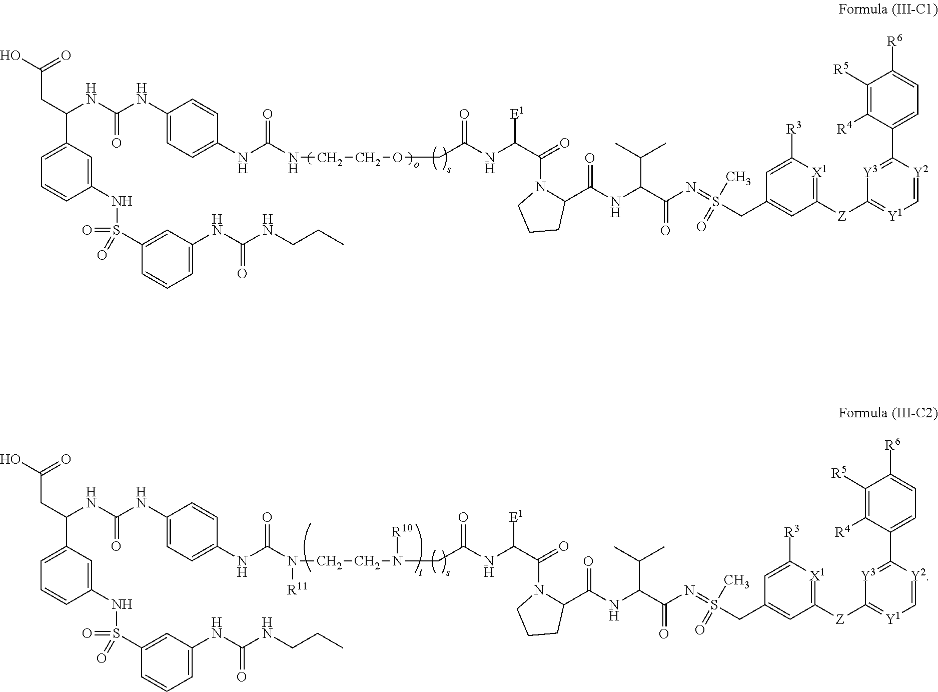

In some embodiments, provided herein is a compound of Formula (II-C1) or Formula (II-C2), or a pharmaceutically acceptable salt thereof, or a stereoisomer or mixture of stereoisomers thereof, wherein:
E¹ is hydrogen, —CH₂C(O)NH₂, —CH₂C(O)OH, —CH₂C(O)OR¹, —CH₂CH₂C(O)OH or —CH₂CH₂C(O)OR¹;
   R¹ is C₁₋₆alkyl substituted with —NH₂ or, —NHL⁶-IN, —N(CH₃)₂, or —N(CH₃)₃⁺;
   wherein:
   L⁶ is substituted C₁₋₃₀ alkyl, or substituted or unsubstituted heteroalkyl; and
   IN is a monovalent radical of an integrin binder;
R¹⁰ is hydrogen or —CH₃;
R¹¹ is hydrogen or —CH₃;
n is 2 to 4;
o is 1 to 8
s is 1 to 4; and
t is 1 to 8.

In some embodiments,
X¹ is CH or N;
Y² is CF or N;
Y³ is CH or N;
R³ is hydrogen;
R⁴ is —OCH₃; or
R³ and R⁴ are taken together to form —O—C₂₋₁₀ alkyl-O— or —O—C₂₋₁₀ alkyl-NH—;
R⁵ is hydrogen; or
R³ and R⁵ are taken together to form —O—C₂₋₁₀ alkyl-O— or —O—C₂₋₁₀ alkyl-NH—;
E¹ is hydrogen, —CH₂C(O)OH or —CH₂C(O)NH₂;
R¹⁰ is —CH₃;
R¹¹ is hydrogen or —CH₃;
n is 2 to 4;
o is 2 to 4;
s is 1 to 4; and
t is 2 to 4.

In some embodiments, provided herein is a compound that is:

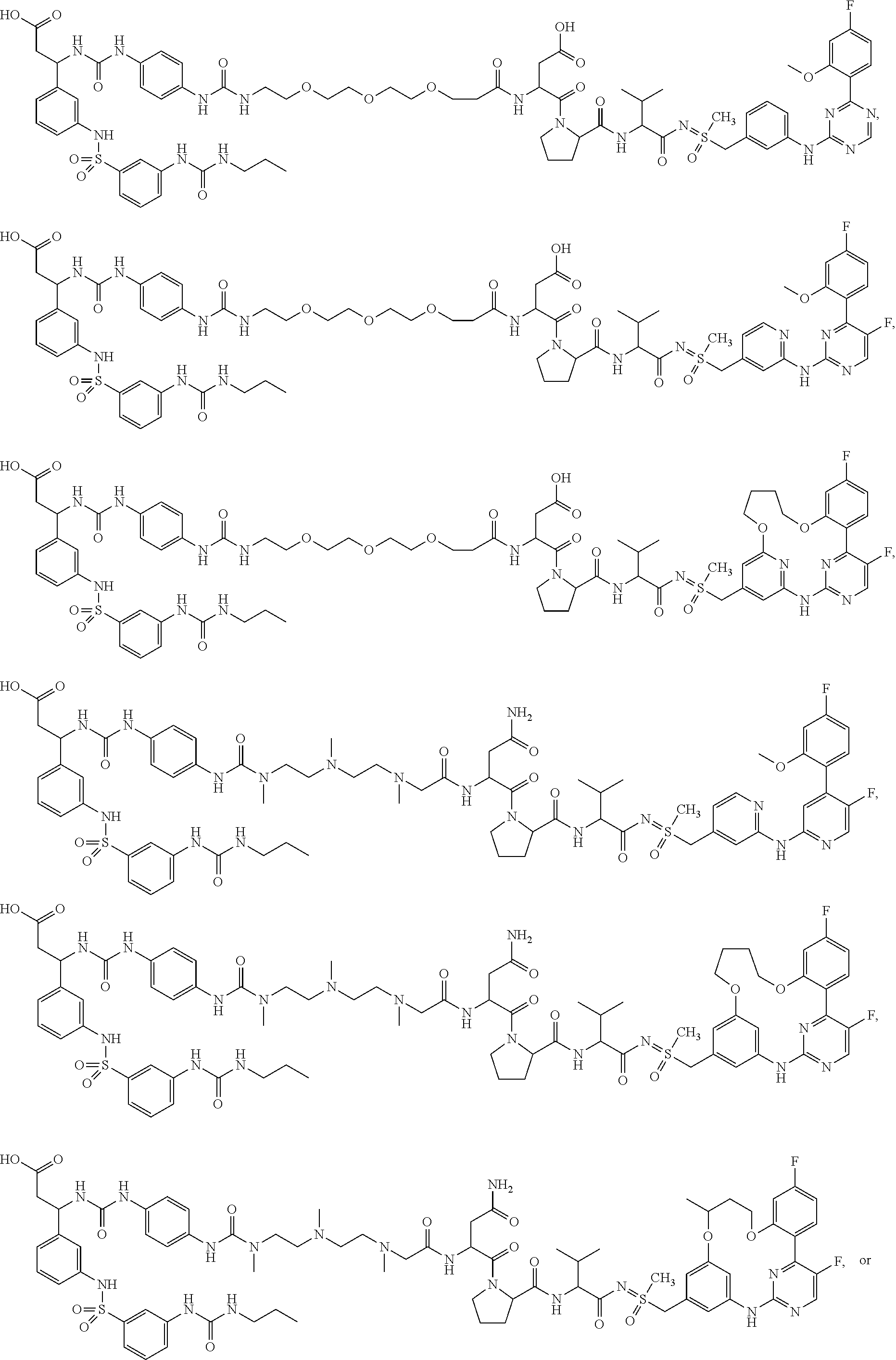

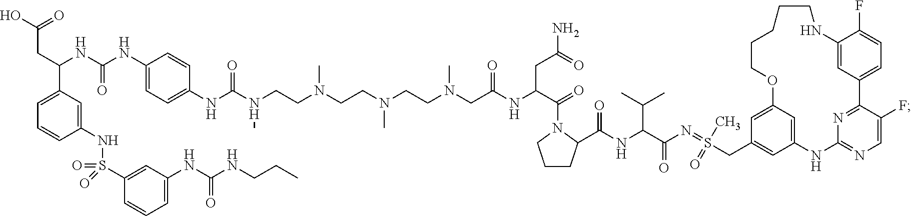 [SMILES: CCCNC(=O)Nc1cccc(S(=O)(=O)Nc2cccc(C(CC(=O)O)NC(=O)Nc3ccc(NC(=O)N(C)CCN(C)CCN(C)CCN(C)CC(=O)NC(CC(N)=O)C(=O)N4CCCC4C(=O)NC(C(=O)N=S(C)(=O)Cc4cc5cc(c4)OCCCCCNc4cc(ccc4F)-c4nc(ncc4F)N5)C(C)C)cc3)c2)c1]

or a pharmaceutically acceptable salt thereof, or a stereoisomer or mixture of stereoisomers thereof.

In some embodiments, provided herein is a compound, or a pharmaceutically acceptable salt thereof, or a stereoisomer or mixture of stereoisomers thereof, having a structure of Formula (V):

IN-L-EL-PT Formula (V)
wherein:
PT is a PTEFb inhibitor;
EL is a peptide linker (e.g., an enzymatically-cleavable linker), optionally further comprising a self-immolative group;
L is a linker (e.g., -L¹A¹(L²-)(L³IN) or L⁵; wherein L¹, L², L³, and L⁵ are bivalent linkers, and A¹ is a trivalent linker); and
IN is an integrin binder.

In some embodiments, EL is a dipeptide having the formula -AA¹-AA²-, or a tripeptide having the formula -AA¹-AA²-AA³-, wherein each AA¹, AA², and AA³ is independently an amino acid, or a derivative thereof. In some embodiments, EL further comprises a self-immolative linker (SIL). In some embodiments, provided herein is a compound of any one of Formulae (01)-(05), wherein EL has the formula:

*-AA¹-AA²-(AA³)₀₋₁-(SIL)₀₋₁-**;
wherein:
each AA¹, AA², and AA³ are independently an amino acid, or a derivative thereof;
SIL is a self-immolative linker;
* is a bond to L (e.g., L¹, L⁴, L⁵, L⁶, or L⁷); and
** is a bond to PT.

In some embodiments, provided herein is a compound wherein EL is:

*-AA¹-AA²-AA³-SIL-** *-AA¹-AA²-AA³-**

*-AA¹-AA²-SIL-**, or *-AA¹-AA²-**

wherein * is a bond to L (e.g., L¹, L⁴, L⁵, L⁶, or L⁷); ** is a bond to PT; AA¹, AA², and AA³ are each independently an amino acid, or a derivative thereof, and SIL is a self-immolative linker.

In some embodiments, provided herein is a compound, or a pharmaceutically acceptable salt thereof; or a stereoisomer or mixture of stereoisomers thereof, having a structure of Formula (V-0):

IN-L-AA¹-AA²-(AA³)₀₋₁-(SIL)₀₋₁-PT Formula (V-0)
wherein:
PT is a PTEFb inhibitor;
SIL is an optional self-immolative linker;
AA¹ is an amino acid;
AA² is an amino acid;
AA³ is an optional amino acid;
L is a linker (e.g., -L¹A¹(L²-)(L³IN) or L⁵; wherein L¹, L², L³, and L⁵ are bivalent linkers, and A¹ is a trivalent linker); and
IN is an integrin binder.

In some embodiments, provided herein is a compound, or a pharmaceutically acceptable salt thereof; or a stereoisomer or mixture of stereoisomers thereof, having a structure of Formula (V-1), Formula (V-2), Formula (V-3), or Formula (V-4):

IN-L-AA¹-AA²-AA³SIL-PT Formula (V-1)

IN-L-AA¹-AA²-AA³-PT Formula (V-2)

IN-L-AA¹-AA²-SIL-PT Formula (V-3)

IN-L-AA¹-AA²-PT Formula (V-4)
wherein:
PT is a PTEFb inhibitor;
SIL is a self-immolative linker;
AA¹ is an amino acid;
AA² is an amino acid;
AA³ is an amino acid;
L is a linker (e.g., -L¹A¹(L²-)(L³IN) or L⁵; wherein L¹, L², L³, and L⁵ are bivalent linkers, and A¹ is a trivalent linker); and
IN is an integrin binder.

In another aspect, provided herein is a compound, or a pharmaceutically acceptable salt thereof; or a stereoisomer or mixture of stereoisomers thereof, having a structure represented by Formula (V-A), Formula (V-C), or Formula (V-E):

PT-EL-L¹-A¹(L²-IN)(L³-IN) Formula (V-A)

PT-EL-L⁵-IN Formula (V-C)

PT-EL-L¹-A¹(L²-IN)(L³-MOD) Formula (V-E)
wherein, in each instance:
PT is a monovalent radical of a PTEFb inhibitor (e.g., a sulfoximine-containing (e.g., sulfoximine-linked) small molecule PTEFb inhibitor (e.g., a macrocyclic PTEFb inhibitor));
EL is a peptide linker (e.g., an enzyme-cleavable linker);
L¹, L², L³, and L⁵ are each linkers (e.g., substituted or unsubstituted C₁₋₃₀ alkyl, or substituted or unsubstituted heteroalkyl linkers);
IN is in each instance, independently, a monovalent radical of an integrin binder;
MOD is a physicochemical or pharmacokinetic modulating group; and
A¹ is a trivalent linker (e.g., a trivalent radical containing 1 to 100 non-hydrogen atoms, optionally containing alkyl, heteroalkyl, carbocyclic, and/or heterocyclic groups, or any combination thereof).

In some embodiments, provided herein is a compound, or a pharmaceutically acceptable salt thereof; or a stereoisomer or mixture of stereoisomers thereof, having a structure of Formula (V-A2), Formula (V-A3), Formula (V-C₂), or Formula (V-C₃):

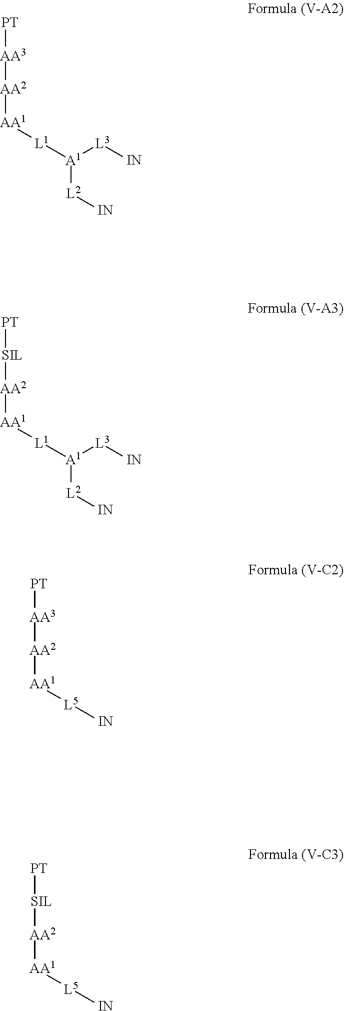

wherein:
PT is a PTEFb inhibitor;
each AA¹, AA², and AA³ is independently an amino acid,
SIL is a self-immolative linker (e.g., a PABC linker);
L¹, L², L³, and L⁵ are bivalent linkers,
A¹ is a trivalent linker; and
IN is an integrin binder.

Each of AA¹ and AA² as used herein can be any naturally occurring or modified amino acid known in the art. For example, in some embodiments, each of AA¹ and AA² is selected from Ala, Arg, Asn, Asp, Cys, Glu, Gln, Gly, His, Ile, Leu, Lys, Met, Phe, Pro, Ser, Thr, Trp, Tyr, Val, Abu (2-aminobutyric acid, or homoalanine), Nva (norvaline), Nle (norleucine), Orn (ornithine), and Cit (citrulline).

In some embodiments, AA¹ is Ala, Leu, Phe, Val. In some embodiments, AA² is Ala, Cit, or Lys. In some embodiments, EL has the formula: -Val-Cit-, -Phe-Cit-, -Leu-Cit-, -Val-Ala-, -Phe-Lys-, -Ala-Lys-, or -Val-Lys-. In some embodiments, EL has the formula: -L-Val-L-Cit-, L-Phe-L-Cit-, -L-Leu-L-Cit-, -L-Val-L-Ala-, -L-Phe-L-Lys-, -L-Ala-L-Lys-, or -L-Val-L-Lys-.

In some embodiments, AA¹ is Ala, Leu, Phe, Val; and AA² is Ala, Cit, or Lys. In some embodiments, EL (i.e., -AA¹-AA²-) is -L-Val-L-Cit-, -L-Phe-L-Cit-, -L-Leu-L-Cit-, -L-Val-L-Ala-, -L-Phe-L-Lys-, -L-Ala-L-Lys-, or -L-Val-L-Lys-. In some embodiments, EL (i.e., -AA¹-AA²-) is -L-Val-L-Cit- (i.e., “EL-2a”).

In some embodiments, SIL is absent. In some embodiments, EL is enzymatically cleavable. In some embodiments, EL is enzymatically cleavable and SIL is absent.

In some embodiments, EL is not enzymatically cleavable. In some embodiments, EL is not enzymatically cleavable, and SIL is present (i.e., conjoins AA² or AA¹ to PT). In some embodiments, SIL is any self-immolative linker known in the art. In some embodiments, SIL is a para-aminophenyl-benzyloxycarbonyl (PABC) group.

In some embodiments, provided herein is a compound, or a pharmaceutically acceptable salt thereof; or a stereoisomer or mixture of stereoisomers thereof, having a structure of Formula (VI-A) or Formula (VI-C):

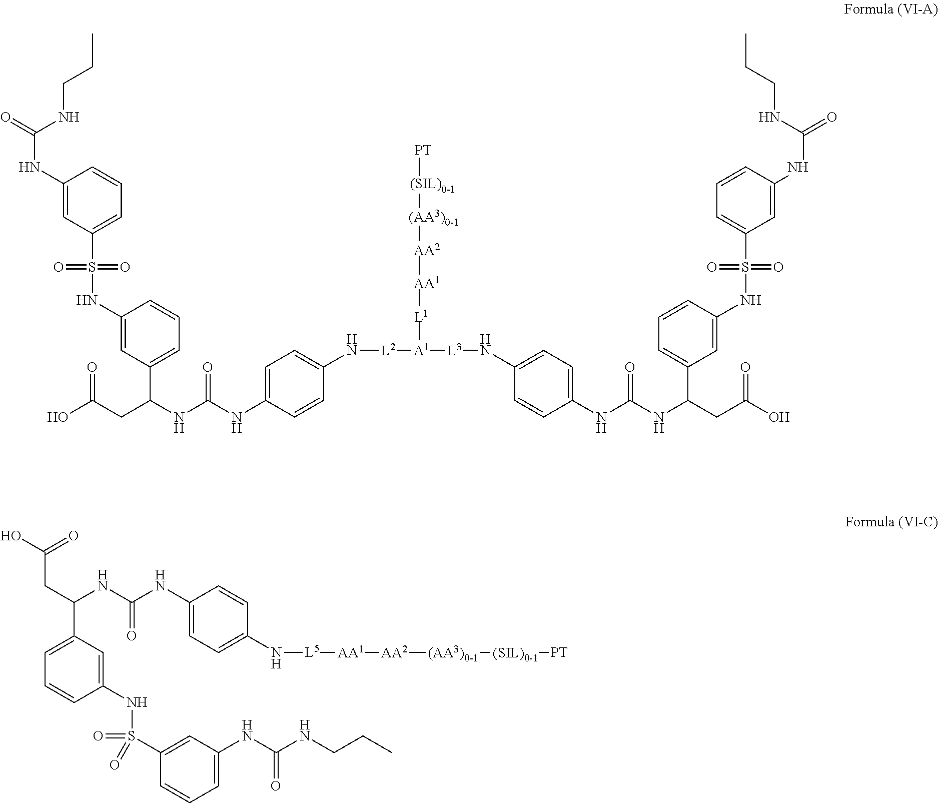

wherein:
PT is a PTEFb inhibitor;
each AA¹, AA², and AA³ is independently an amino acid,
SIL is a self-immolative linker (e.g., a PABC linker);
L¹, L², L³, and L⁵ are bivalent linkers,
A¹ is a trivalent linker; and
IN is an integrin binder.

In some embodiments, provided herein is a compound of Formula (VI-A) or Formula (VI-C), or a pharmaceutically acceptable salt thereof; or a stereoisomer or mixture of stereoisomers thereof, wherein:
PT is a PTEFb inhibitor;
SIL is a self-immolative linker;
AA¹ is Gly, Ala, Asp, Asn, Leu, Phe, or Val;
AA² is Ala, Cit, Lys, or Pro;
AA³ is absent, Gly, Ala, Val, Asn, or Asp;
L⁵ is a linker (e.g., a simple spacer or a polymeric spacer, defined herein).

In some embodiments, provided herein is a compound, or a pharmaceutically acceptable salt thereof; or a stereoisomer or mixture of stereoisomers thereof, having a structure of Formula (VI-C1) or Formula (VI-C2):

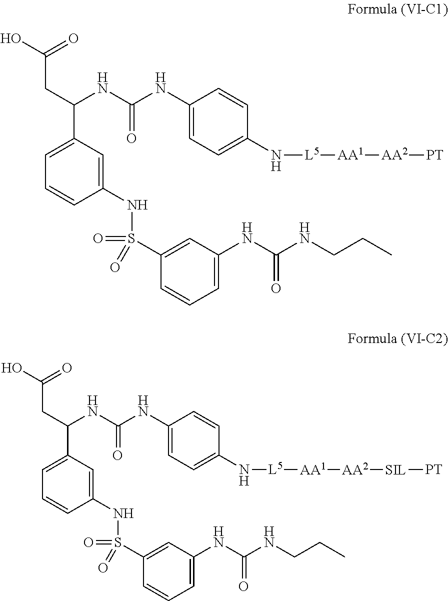

wherein:
PT is a PTEFb inhibitor;
SIL is a self-immolative linker;
AA¹ is Ala, Leu, Phe, or Val;
AA² is Ala, Cit, or Lys; and
L⁵ is a linker (e.g., a simple spacer or a polymeric spacer, defined herein).

In some embodiments, provided herein is a compound of Formula (VI-A) or Formula (VI-C), or a pharmaceutically acceptable salt thereof; or a stereoisomer or mixture of stereoisomers thereof, wherein:
PT is a PTEFb inhibitor;
AA¹ is L-Ala, L-Leu, L-Phe, or L-Val;
AA² is L-Ala, L-Cit, or L-Lys; and
L⁵ is a substituted or unsubstituted alkyl, or substituted or unsubstituted heteroalkyl.

In some embodiments, -AA¹-AA²- is a dipeptide having the sequence: -L-Val-L-Cit-, -L-Phe-L-Cit-, -L-Leu-L-Cit-, -L-Val-L-Ala-, -L-Phe-L-Lys-, -L-Ala-L-Lys-, or -L-Val-L-Lys-.

In some embodiments, provided herein is a compound of Formula (VI-C), or a pharmaceutically acceptable salt thereof; or a stereoisomer or mixture of stereoisomers thereof, wherein:
SIL is a self-immolative linker; AA¹ is L-Val; and AA² is L-Ala or L-Cit; or
SIL is absent; AA¹ is L-Val; and AA² is L-Cit; and
L⁵ is a substituted or unsubstituted alkyl, or substituted or unsubstituted heteroalkyl.

In some embodiments, L⁵ is substituted or unsubstituted heteroalkyl. In some embodiments, L⁵ is a linker having a structure represented by the formula:

—(CO)ₘ(CH₂)ₙ(OC₂₋₆ alkyl)ₒ(NIH)ₚ(CO)_{q}—; (i)

—(CO)ᵣ(CH₂)ₛ(NR¹⁰C₂₋₆ alkyl)ₜ(NR¹¹)ᵤ(CO)ᵥ—; or (ii)

—(CO)ᵣ(CH₂)ₛ(NR¹⁰C(O)C₁₋₆ alkyl)ₜ(NR¹¹)ᵤ(CO)ᵥ—; (iii)
wherein:
   R¹⁰ is hydrogen or C₁₋₃ alkyl;
   R¹¹ is hydrogen or C₁₋₃ alkyl;
   m is 0 or 1;
   n is 0 to 10;
   is 1 to 10;
   p is 0 or 1;
   q is 0 or 1;
   r is 0 or 1;
   s is 0 to 10;
   t is 1 to 10;
   u is 0 or 1; and
   v is 0 or 1.

In some embodiments, provided herein is a compound, or a pharmaceutically acceptable salt thereof; or a stereoisomer or mixture of stereoisomers thereof, wherein:
L⁵ is a linker having a structure represented by the formula:

—(CO)ₘ(CH₂)ₙ(OC₂₋₆ alkyl)ₒ(NH)ₚ(CO)_{q};
wherein:
m is 0 or 1;
n is 2 to 4;
is 1 to 8;
p is 0 or 1;
q is 0 or 1;

In some embodiments, provided herein is a compound, or a pharmaceutically acceptable salt thereof; or a stereoisomer or mixture of stereoisomers thereof, wherein:
L⁵ is a linker having a structure represented by the formula:

—(CO)ᵣ(CH₂)ₛ(NR¹⁰C₂₋₆ alkyl)ₜ(NR¹¹)ᵤ(CO)ᵥ—; or

—(CO)ᵣ(CH₂)ₛ(NR¹⁰C(O)C₁₋₆ alkyl)ₜ(NR¹¹)ᵤ(CO)ᵥ—;
wherein:
R¹⁰ is hydrogen or CH₃;
R¹¹ is hydrogen or CH₃;
r is 0 or 1;
s is 1 to 4;
t is 1 to 8;
u is 0 or 1; and
v is 0 or 1.

In some embodiments, SIL is a PABC linker. In some embodiments, SIL is

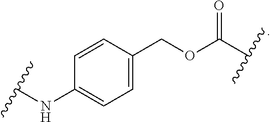 [SMILES: *Nc1ccc(COC(*)=O)cc1]

In some embodiments, PT is a radical of a PTEFb inhibitor having a structure represented by the formula:

—(SFX)-(L^{A})-(Ring A)-(L^{B})-(Ring B)-(L^{C})-(Ring C)-(L^{D})-; (iv)
wherein:
SFX is a sulfoximine moiety (e.g., —N═S(═O)(C₁₋₆alkyl)-)
L^{A} is a bond or C₁₋₆alkyl;
Ring A is an optionally substituted carbocycle or optionally substituted heterocycle (e.g., optionally substituted phenyl or optionally substituted heteroaryl);
L^{B} is a bond, optionally substituted C₁₋₆alkyl, or optionally substituted heteroalkyl (e.g., —CH₂—, —C(═O)NH— —NH—, —N(CH₃)—, —NHC(═O)—, —O—, or —S—;);
Ring B is an optionally substituted carbocycle or optionally substituted heterocycle (e.g., optionally substituted phenyl or optionally substituted heteroaryl);
L^{C} is a bond, optionally substituted C₁₋₆alkyl, or optionally substituted heteroalkyl (e.g., —CH₂—, —C(═O)NH— —NH—, —N(CH₃)—, —NHC(═O)—, —O—, or —S—;);
Ring C is an optionally substituted carbocycle or optionally substituted heterocycle (e.g., optionally substituted phenyl or optionally substituted heteroaryl); and
L^{D} is absent; or an optionally substituted heteroalkyl bonded to Ring A, thereby forming an optionally substituted macrocyclic Ring D (e.g., a ring comprising 12 to 20 atoms selected from C, N, O, and S).

In some embodiments, PT is a PTEFb inhibitor. In some embodiments, PT is a radical of a PTEFb inhibitor having a structure represented by the formula:

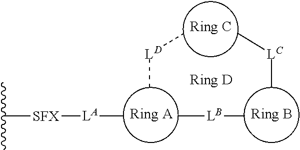

wherein:
SFX is a sulfoximine moiety;
L^{A} is a bond or C₁₋₆alkyl;
Ring A is an optionally substituted phenyl or optionally substituted heteroaryl;
L^{B} is —CH₂—, —C(═O)NH— —NH—, —N(CH₃)—, —NHC(═O)—, —O—, or —S—;
Ring A is an optionally substituted phenyl or optionally substituted heteroaryl;
L^{C} is a bond or —CH₂—, —C(═O)NH— —NH—, —N(CH₃)—, —NHC(═O)—, —O—, or —S—; Ring C is an optionally substituted phenyl or optionally substituted heteroaryl; and
L^{D} is absent; or an optionally substituted heteroalkyl bonded to Ring A.

In some embodiments, provided herein is a compound, or a pharmaceutically acceptable salt thereof; or a stereoisomer or mixture of stereoisomers thereof, having a structure of Formula (VII-C):

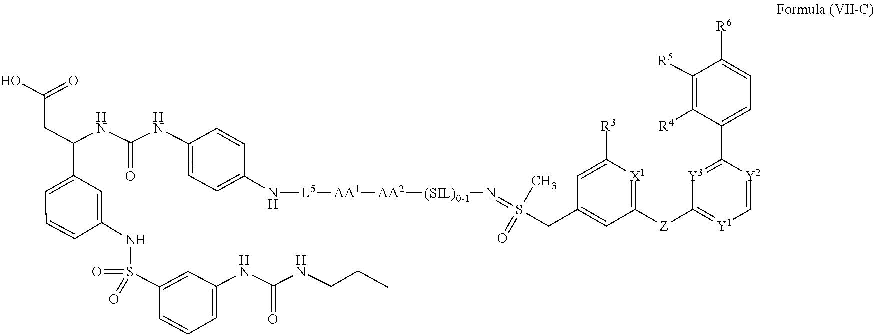

wherein:
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl;
or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl;
or R³ and R⁵ are taken together to form a bivalent radical of the formula —O—C₂₋₁₀ alkyl-O—, —O—C₂₋₁₀ alkyl-NH—, or —NH—C₂₋₁₀ alkyl-NH—;
R⁶ is hydrogen, halogen, —OH, or —O—C₁₋₄alkyl; SIL is a self-immolative linker; and
L⁵ is a linker (e.g., a simple spacer or a polymeric spacer, defined herein).

In some embodiments, L⁵ is:

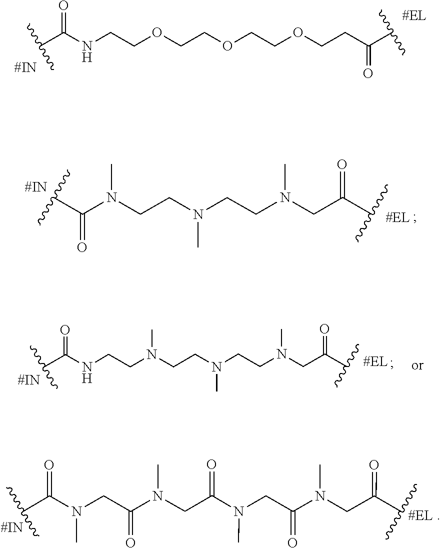 [SMILES: *C(=O)CCOCCOCCOCCNC(*)=O ; *C(=O)CN(C)CCN(C)CCN(C)C(*)=O ; *C(=O)CN(C)CCN(C)CCN(C)CCNC(*)=O ; *C(=O)CN(C)C(=O)CN(C)C(=O)CN(C)C(=O)CN(C)C(*)=O]

In some embodiments, provided herein is a compound, or a pharmaceutically acceptable salt thereof; or a stereoisomer or mixture of stereoisomers thereof, having a structure of Formula (VII-C1), Formula (VII-C2), or Formula (VII-C3):

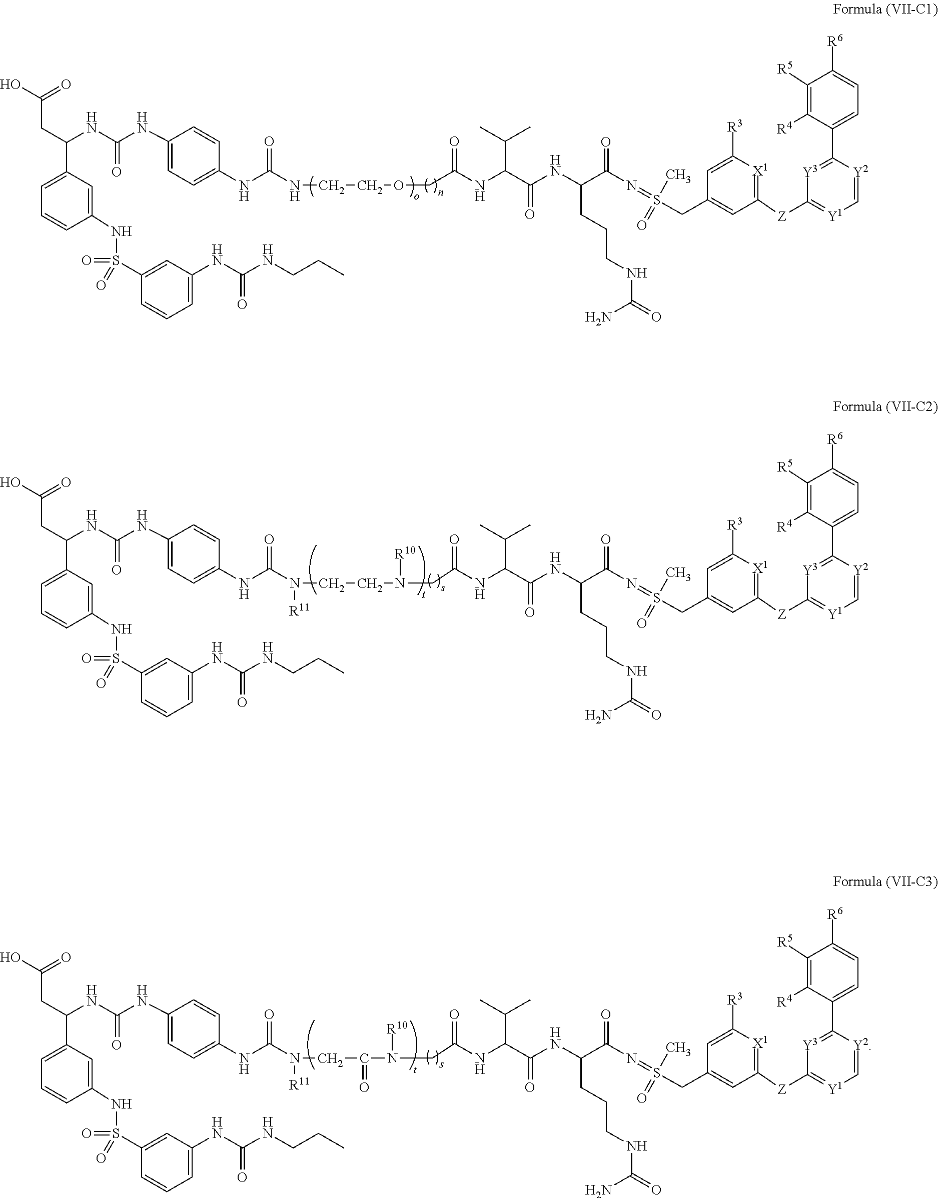

In some embodiments, provided herein is a compound that is:

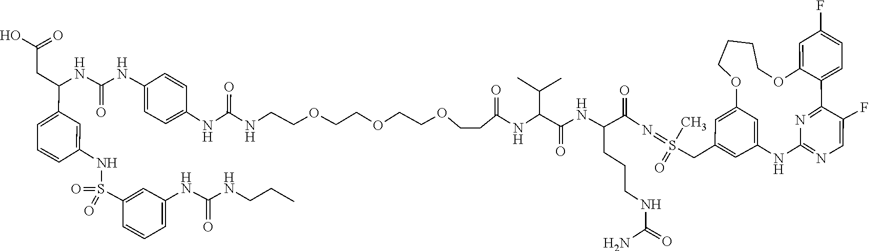 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(C(=O)NC(CCCNC(N)=O)C(=O)N=S(C)(=O)Cc4cc5cc(c4)OCCCCOc4cc(F)ccc4-c4nc(ncc4F)N5)C(C)C)cc3)c2)c1]

or a pharmaceutically acceptable salt thereof; or a stereoisomer or mixture of stereoisomers thereof.

In some embodiments, provided herein is a compound of Formula (VII-C4), Formula (VII-C5), or Formula (VII-C6)

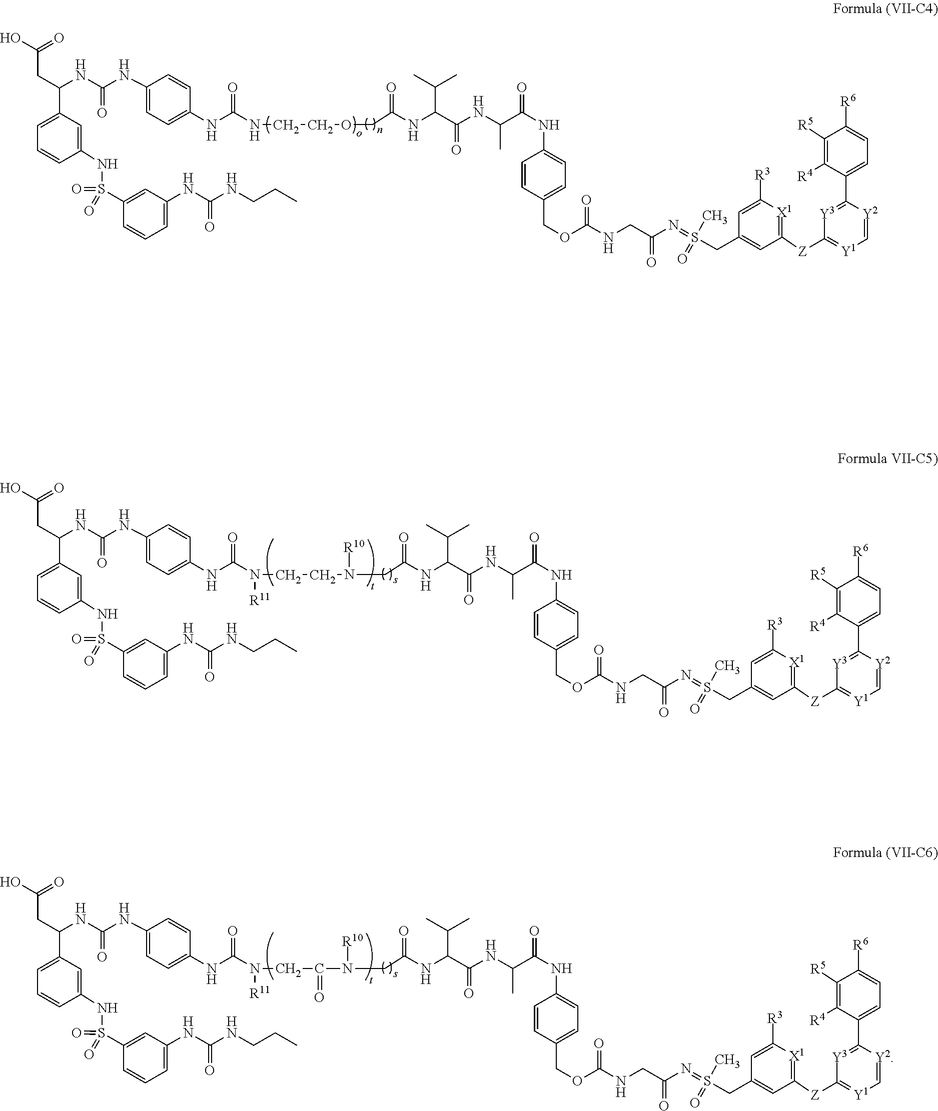

or a pharmaceutically acceptable salt thereof; or a stereoisomer or mixture of stereoisomers thereof, wherein:
SIL is

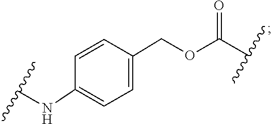 [SMILES: *Nc1ccc(COC(*)=O)cc1]

X¹ is CH or N;
Y¹ is N;
Y² is CF or N;
Y³ is CH or N;
Z is —NH—;
R³ is hydrogen;
R⁴ is —OCH₃; or
R³ and R⁴ are taken together to form —O—C₂₋₁₀ alkyl-O— or —O—C₂₋₁₀ alkyl-NH—;
R⁵ is hydrogen; or
R³ and R⁵ are taken together to form —O—C₂₋₁₀ alkyl-O— or —O—C₂₋₁₀ alkyl-NH—;
R⁶ is halogen;
R¹⁰ is —CH₃;
R¹¹ is hydrogen or —CH₃;
n is 2 to 4;
o is 1 to 8;
s is 1 to 4; and
t is 1 to 8.

In some embodiments, provided herein is a compound, or a pharmaceutically acceptable salt thereof, or a stereoisomer or mixture of stereoisomers thereof, having the structure:

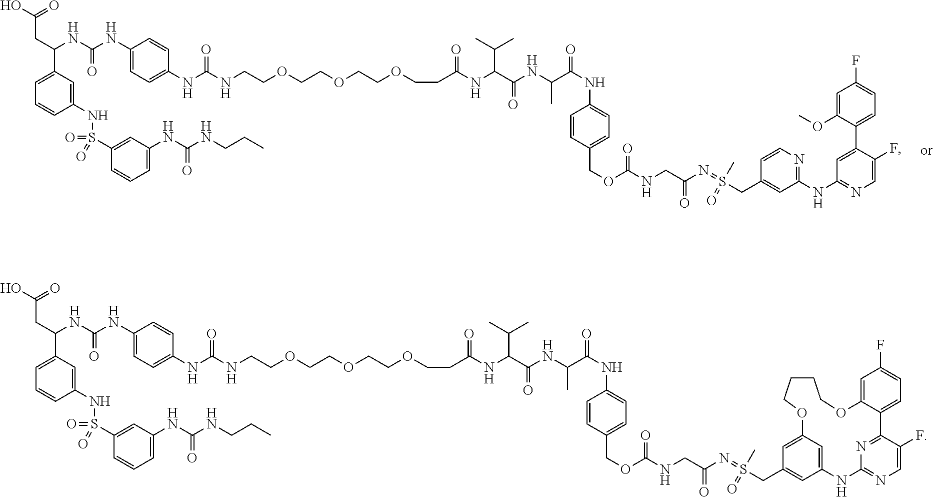 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(C(=O)NC(C)C(=O)Nc4ccc(COC(=O)NCC(=O)N=S(C)(=O)Cc5ccnc(Nc6cc(-c7ccc(F)cc7OC)c(F)cn6)c5)cc4)C(C)C)cc3)c2)c1 ; CCCNC(=O)Nc1cccc(S(=O)(=O)Nc2cccc(C(CC(=O)O)NC(=O)Nc3ccc(NC(=O)NCCOCCOCCOCCC(=O)NC(C(=O)NC(C)C(=O)Nc4ccc(COC(=O)NCC(=O)N=S(C)(=O)Cc5cc6cc(c5)OCCCCOc5cc(F)ccc5-c5nc(ncc5F)N6)cc4)C(C)C)cc3)c2)c1]

In some embodiments, provided herein is a compound, or a pharmaceutically acceptable salt thereof, or a stereoisomer or mixture of stereoisomers thereof, having the structure:

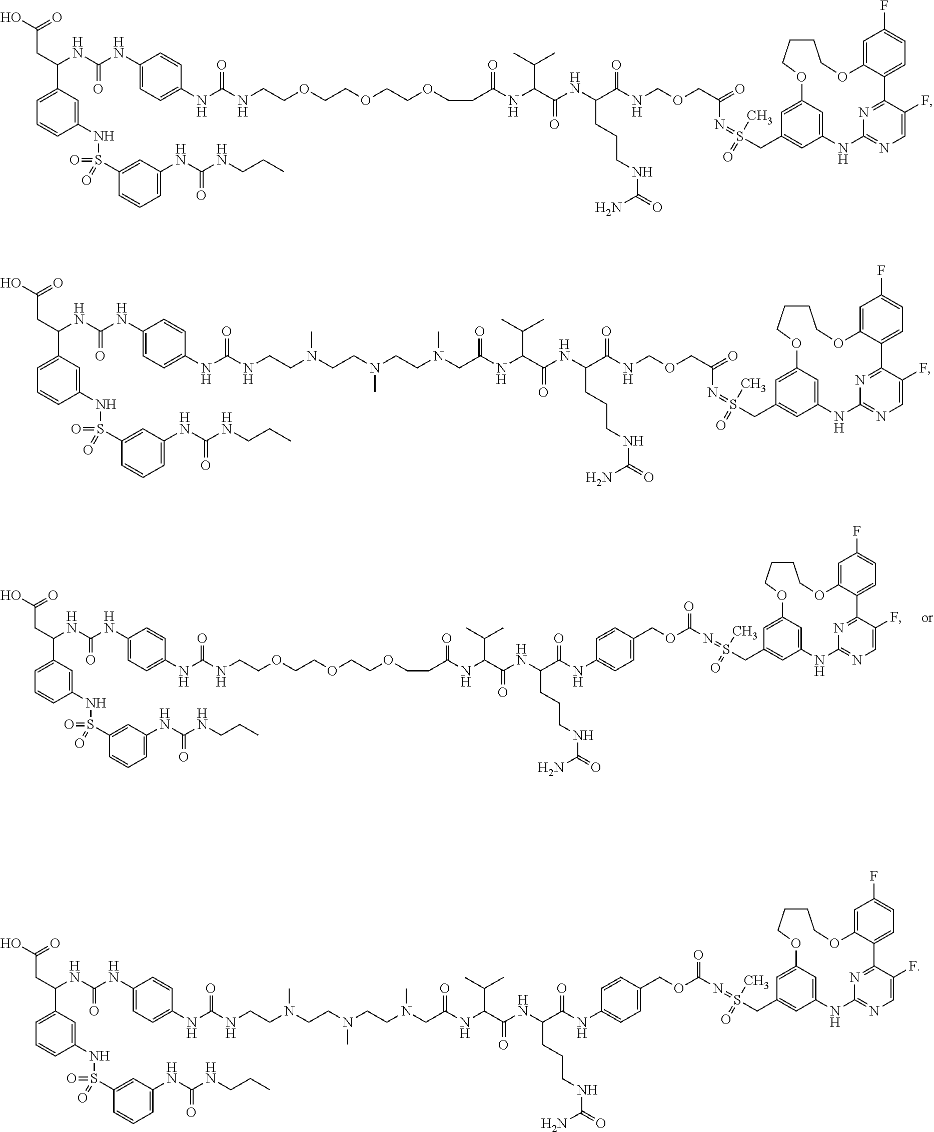

In another aspect, provided herein is a compound, or a pharmaceutically acceptable salt thereof; or a stereoisomer or mixture of stereoisomers thereof, having a structure represented by Formula (A), Formula (C), or Formula (E):

PT-EL-L¹-A(L²-IN)(L³-IN) Formula (A)

PT-EL-L⁵-IN Formula (C)

PT-EL-L¹-A¹(L²-IN)(L³-MOD) Formula (E)
wherein, in each instance:
PT is a monovalent radical of a PTEFb inhibitor (e.g., a sulfoximine-containing (e.g., sulfoximine-linked) small molecule PTEFb inhibitor (e.g., a macrocyclic PTEFb inhibitor));
EL is a legumain-cleavable linker;
L¹, L², L³, are each linkers (e.g., substituted or unsubstituted C₁₋₃₀ alkyl, or substituted or unsubstituted heteroalkyl linkers);
L⁵ is a linker (e.g., a substituted C₁₋₃₀ alkyl, or substituted or unsubstituted heteroalkyl);
IN is in each instance, independently, a monovalent radical of an integrin binder;
MOD is a physicochemical or pharmacokinetic modulating group; and
A¹ is a trivalent linker (e.g., a trivalent radical containing 1 to 100 non-hydrogen atoms, optionally containing alkyl, heteroalkyl, carbocyclic, and/or heterocyclic groups, or any combination thereof).

In some embodiments, EL is a tripeptide, having the formula -AA¹-AA²-AA³-, wherein each AA¹, AA², and AA³ is independently an amino acid (including D-amino acids and/or N-alkyl amino acids).

Each of AA¹, AA², and AA³ as used herein can be any naturally occurring or modified amino acid known in the art. For example, in some embodiments, each of AA¹, AA², and AA³ is selected from Ala, Arg, Asn, Asp, Cys, Glu, Gln, Gly, His, Ile, Leu, Lys, Met, Phe, Pro, Ser, Thr, Trp, Tyr, Val, Abu (2-aminobutyric acid, or homoalanine), Nva (norvaline), Nle (norleucine), Orn (ornithine), and Cit (citrulline).

In some embodiments, the present invention provides compounds having a structure of formulae disclosed herein, or a pharmaceutically acceptable salt thereof, or a stereoisomer or mixture of stereoisomers thereof, wherein EL is a dipeptide, having the formula -AA¹-AA²-. In some embodiments, EL is a dipeptide having the formula -AA¹-AA²-, wherein each AA¹ and AA² is independently an amino acid (including D-amino acids and/or N-alkyl amino acids).

Each of AA¹ and AA² as used herein can be any naturally occurring or modified amino acid known in the art. For example, in some embodiments, each of AA¹ and AA² is selected from Ala, Arg, Asn, Asp, Cys, Glu, Gln, Gly, His, Ile, Leu, Lys, Met, Phe, Pro, Ser, Thr, Trp, Tyr, Val, Abu (2-aminobutyric acid, or homoalanine), Nva (norvaline), Nle (norleucine), Orn (ornithine), and Cit (citrulline). As used herein, a formulae depicting AA¹, AA², and AA³ may be a dipeptide, wherein AA³ is absent.

In some embodiments, each of AA¹, AA², and AA³ is an optionally N-alkylated (e.g., N-methylated) amino acid. In some embodiments, one of AA¹, AA², and AA³ is an N-alkylated (e.g., N-methylated) amino acid. In some embodiments, one of AA¹, AA², and AA³ is N-methyl alanine. In some embodiments, EL is cleaved by legumain more selectively when one of AA¹, AA², and AA³ is N-alkylated (e.g., N-methylated), such as when AA² is N-methyl Ala (e.g., N-methyl L-Ala).

In some embodiments, AA¹ is Ala, N-methyl (“N-Me) Ala, or Asp. In some embodiments, AA² is Ala, N-Me Ala, Asp, Asn, His, or Ser. In some embodiments, AA¹ is L-Ala, N-Me L-Ala, or L-Asp. In some embodiments, AA² is L-Ala, N-Me L-Ala, D-Ala, N-Me D-Ala, L-Asp, D-Asp, L-Asn, D-Asn, L-His, D-His, L-Ser, or D-Ser. In some embodiments, AA¹ is L-Ala or L-Asp. In some embodiments, AA² is L-Ala, N-Me L-Ala, D-Ala, L-Asp, D-Asp, L-Asn, D-His, or D-Ser. In some embodiments, AA³ is Asp or Asn. In some embodiments, AA³ is Asp. In some embodiments, AA³ is Asn. In some embodiments, AA³ is L-Asn. In some embodiments, AA³ is L-Asp. In some embodiments, AA³ is L-Asp or L-Asn.

In some embodiments, EL has the formula: -L-Ala-L-Ala-L-Asp-, -L-Ala-L-Ala-L-Asn-, -L-Ala-L-Asp-L-Asn-, -L-Ala-L-Asn-, or -L-Asp-L-Asn-, wherein each amino acid is, independently of one another, optionally, N-alkylated with C₁₋₃ alkyl.

In some embodiments, EL has the formula: -L-Ala-L-N-Me-Ala-L-Asn-, -L-Ala-D-His-L-Asn-, -L-Ala-D-Asp-L-Asn-, -L-Ala-D-Ala-L-Asn-, or -L-Ala-D-Ser-L-Asn-.

In some embodiments, provided herein is a compound, or a pharmaceutically acceptable salt thereof; or a stereoisomer or mixture of stereoisomers thereof, having a structure of Formula (X-A) or Formula (X-C):

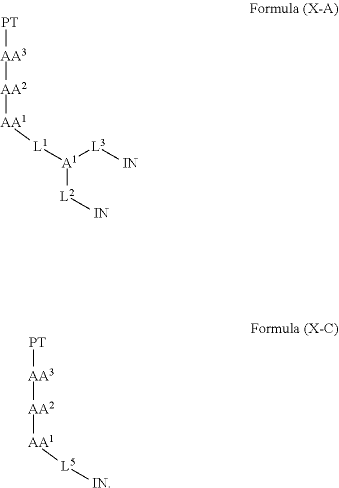

In some embodiments, provided herein is a compound, or a pharmaceutically acceptable salt thereof; or a stereoisomer or mixture of stereoisomers thereof, having a structure of Formula (X-A), Formula (X-B), Formula (X-C) or Formula (X-D), wherein PT, AA³, AA², AA¹, L¹, A¹, L², L³, L⁴, L⁵, L⁷, and IN are each as described herein.

In some embodiments, provided herein is a compound, or a pharmaceutically acceptable salt thereof; or a stereoisomer or mixture of stereoisomers thereof, having a structure of Formula (X-C) wherein:
each AA¹, AA², and AA³ is independently an amino acid, and
L⁵ is a substituted or unsubstituted alkyl, or substituted or unsubstituted heteroalkyl.

In some embodiments, provided herein is a compound, or a pharmaceutically acceptable salt thereof; or a stereoisomer or mixture of stereoisomers thereof, having a structure of Formula (X-C) wherein:
each AA¹ and AA² is independently an amino acid, AA³ is absent; and
L⁵ is a substituted or unsubstituted alkyl, or substituted or unsubstituted heteroalkyl.

In some embodiments, EL (i.e., -AA¹-AA²-AA³-) is -L-Ala-L-Ala-L-Asp-, -L-Ala-L-Ala-L-Asn-, -L-Ala-L-Asp-L-Asn-, -L-Ala-L-Asn-, -L-Asp-L-Asn-, -L-Ala-N-Me L-Ala-L-Asn-, -L-Ala-D-His-L-Asn-, -L-Ala-D-Asp-L-Asn-, -L-Ala-D-Ala-L-Asn-, or -L-Ala-D-Ser-L-Asn-. In some embodiments, EL (i.e., -AA¹-AA²-AA³-) is -L-Ala-L-Ala-L-Asp-, -L-Ala-L-Ala-L-Asn-, -L-Ala-L-Asp-L-Asn, -L-Ala-L-Asn-, or -L-Asp-L-Asn-. In some embodiments, EL (i.e., -AA¹-AA²-AA³-) is -L-Ala-N-Me L-Ala-L-Asn-, -L-Ala-D-His-L-Asn-, -L-Ala-D-Asp-L-Asn-, -L-Ala-D-Ala-L-Asn-, or -L-Ala-D-Ser-L-Asn-. In some embodiments, EL (i.e., -AA¹-AA²-AA³-) is -L-Ala-N-Me L-Ala-L-Asn- (i.e., “EL-3a”).

In some embodiments, provided herein is a compound, or a pharmaceutically acceptable salt thereof; or a stereoisomer or mixture of stereoisomers thereof, having a structure of Formula (XI-C):

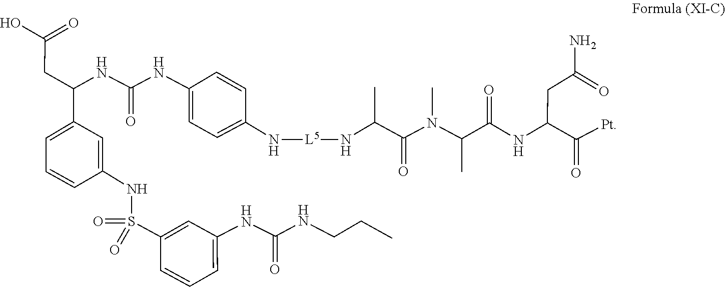

In some embodiments, provided herein is a compound of Formula (XI-C), or a pharmaceutically acceptable salt thereof; or a stereoisomer or mixture of stereoisomers thereof, wherein:
PT is a monovalent radical of a PTEFb inhibitor;
L⁵ is a substituted or unsubstituted alkyl, or substituted or unsubstituted heteroalkyl.

In some embodiments, L⁵ is substituted or unsubstituted heteroalkyl. In some embodiments, L⁵ is a linker having a structure represented by the formula:

—(CO)ₘ(CH₂)ₙ(OC₂₋₆ alkyl)ₒ(NH)ₚ(CO)_{q}—; (i)

—(CO)ᵣ(CH₂)ₛ(NR¹⁰C₂₋₆ alkyl)ₜ(NR¹¹)ᵤ(CO)ᵥ—; or (ii)

—(CO)ᵣ(CH₂)ₛ(NR¹⁰C(O)C₁₋₆ alkyl)ₜ(NR¹¹)ᵤ(CO)ᵥ—; (iii)
wherein:
   R¹⁰ is hydrogen or C₁₋₃ alkyl;
   R¹¹ is hydrogen or C₁₋₃ alkyl;
   m is 0 or 1;
   n is 0 to 10;
   is 1 to 10;
   p is 0 or 1;
   q is 0 or 1;
   r is 0 or 1;
   s is 0 to 10;
   t is 1 to 10;
   u is 0 or 1; and
   v is 0 or 1.

In some embodiments, provided herein is a compound, or a pharmaceutically acceptable salt thereof; or a stereoisomer or mixture of stereoisomers thereof, wherein:
L⁵ is a linker having a structure represented by the formula:

—(CO)ₘ(CH₂)ₙ(OC₂₋₆ alkyl)ₒ(NH)ₚ(CO)_{q};
wherein:
m is 0 or 1;
n is 2 to 4;
is 1 to 8;
p is 0 or 1;
q is 0 or 1;
In some embodiments, provided herein is a compound, or a pharmaceutically acceptable salt thereof; or a stereoisomer or mixture of stereoisomers thereof, wherein:
L⁵ is a linker having a structure represented by the formula:

—(CO)ᵣ(CH₂)ₛ(NR¹⁰C₂₋₆ alkyl)ₜ(NR¹¹)ᵤ(CO)ᵥ—;
wherein:
R¹⁰ is hydrogen or CH₃;
R¹¹ is hydrogen or CH₃;
r is 0 or 1;
s is 1 to 4;
t is 1 to 8;
u is 0 or 1; and
v is 0 or 1.

In some embodiments, provided herein is a compound, or a pharmaceutically acceptable salt thereof; or a stereoisomer or mixture of stereoisomers thereof, having a structure of Formula (XI-C1) or Formula (XI-C2):

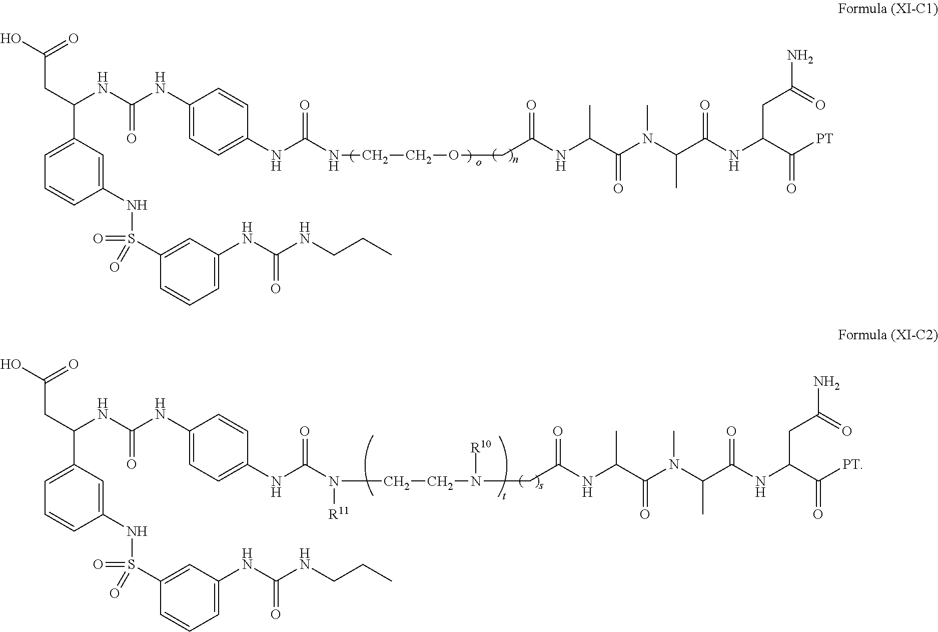

In some embodiments, PT is a radical of a PTEFb inhibitor having a structure represented by the formula:

—(SFX)-(L^{A})-(Ring A)-(L^{B})-(Ring B)-(L^{C})-(Ring C)-(L^{D})-; (iv)
wherein:
SFX is a sulfoximine moiety (e.g., —N═S(═O)(C₁₋₆alkyl)-)
L^{A} is a bond or C₁₋₆alkyl;
Ring A is an optionally substituted carbocycle or optionally substituted heterocycle (e.g., optionally substituted phenyl or optionally substituted heteroaryl);
L^{B} is a bond, optionally substituted C₁₋₆alkyl, or optionally substituted heteroalkyl (e.g., —CH₂—, —C(═O)NH— —NH—, —N(CH₃)—, —NHC(═O)—, —O—, or —S—;);
Ring B is an optionally substituted carbocycle or optionally substituted heterocycle (e.g., optionally substituted phenyl or optionally substituted heteroaryl);
L^{C} is a bond, optionally substituted C₁₋₆alkyl, or optionally substituted heteroalkyl (e.g., —C(═O)NH— —NH—, —N(CH₃)—, —NHC(═O)—, —O—, or —S—);
Ring C is an optionally substituted carbocycle or optionally substituted heterocycle (e.g., optionally substituted phenyl or optionally substituted heteroaryl); and
L^{D} is absent; or an optionally substituted heteroalkyl bonded to Ring A, thereby forming an optionally substituted macrocyclic Ring D (e.g., a ring comprising 12 to 20 atoms selected from C, N, O, and S).

In some embodiments, PT has a structure represented by the formula:

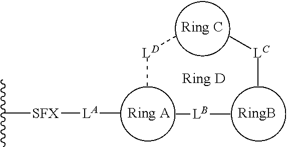

wherein if L^{D} is present, it connects Ring C and Ring A to form a macrocyclic Ring D.

In some embodiments, LD is absent. In some embodiments, LD is an optionally substituted heteroalkyl group. In some embodiments, LD is a heteroalkyl group comprising 2 to 12 atoms selected from C, N, O, and S. In some embodiments, LD is a heteroalkyl group of the formula —O—(C₁₋₆alkyl)-O—, —NH—(C₁₋₆alkyl)-O—, or —NH—(C₁₋₆alkyl)-NH—. In some embodiments, L^{D} is a heteroalkyl group of the formula —O—(C₁₋₆alkyl)-O—. In some embodiments, L^{C} is a bond. In some embodiments, L^{B} is —CH₂—, —C(═O)NH— —NH—, —N(CH₃)—, —NHC(═O)—, —O—, or —S—. In some embodiments, L^{B} is —CH₂—, —NH—, —N(CH₃)—, —O—, or —S—. In some embodiments, L^{B} is —CH₂—, —NH—, —O—, or —S—. In some embodiments, L^{B} is —CH₂—. In some embodiments, L^{B} is —NH—. In some embodiments, L^{B} is —O—. In some embodiments, L^{A} is C₁₋₆ alkyl. In some embodiments, L^{A} is —CH₂.

In some embodiments, provided herein is a compound, or a pharmaceutically acceptable salt thereof; or a stereoisomer or mixture of stereoisomers thereof, having a structure of Formula (XII-C)

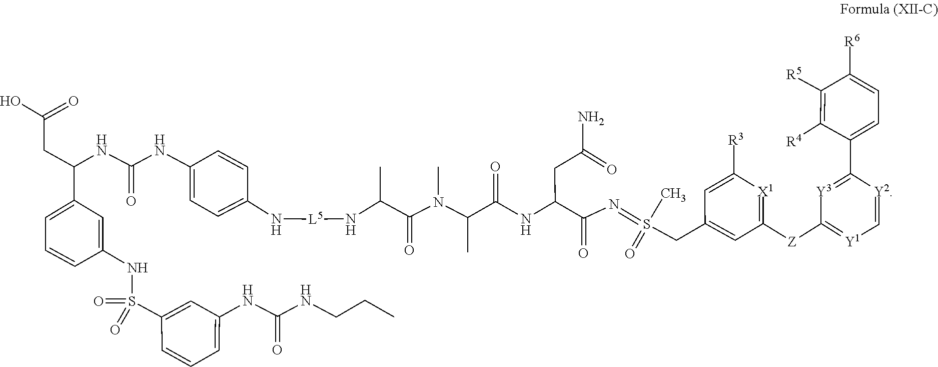

In some embodiments, provided herein is a compound, or a pharmaceutically acceptable salt thereof; or a stereoisomer or mixture of stereoisomers thereof, having a structure of Formula (XII-C), wherein:
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl;
or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl;
or R³ and R⁵ are taken together to form a bivalent radical of the formula —O—C₂₋₁₀ alkyl-O—, —O—C₂₋₁₀ alkyl-NH—, or —NH—C₂₋₁₀ alkyl-NH—;
R⁶ is hydrogen, halogen, —OH, or —O—C₁₋₄alkyl; and
L⁵ is a substituted or unsubstituted alkyl, or substituted or unsubstituted heteroalkyl.

In some embodiments, provided herein is a compound, or a pharmaceutically acceptable salt thereof; or a stereoisomer or mixture of stereoisomers thereof, wherein L⁵ is a linker having a structure represented by the formula:

—(CO)ₘ(CH₂)ₙ(OC₂₋₆ alkyl)ₒ(NH)ₚ(CO)_{q}—; (i)

—(CO)ᵣ(CH₂)ₛ(NR¹⁰C₂₋₆ alkyl)ₜ(NR¹¹)ᵤ(CO)ᵥ—; or (ii)

—(CO)ᵣ(CH₂)ₛ(NR¹⁰C(O)C₁₋₆ alkyl)ₜ(NR¹¹)ᵤ(CO)ᵥ—; (iii)
wherein:
R¹⁰ is hydrogen or CH₃;
R¹¹ is hydrogen or CH₃;
m is 0 or 1;
n is 2 to 4;
o is 1 to 8;
p is 0 or 1;
q is 0 or 1;
r is 0 or 1;
s is 1 to 4;
t is 1 to 8;
u is 0 or 1; and
v is 0 or 1.

In some embodiments, provided herein is a compound, or a pharmaceutically acceptable salt thereof; or a stereoisomer or mixture of stereoisomers thereof, having a structure of Formula (XII-C1) or Formula (XII-C2):

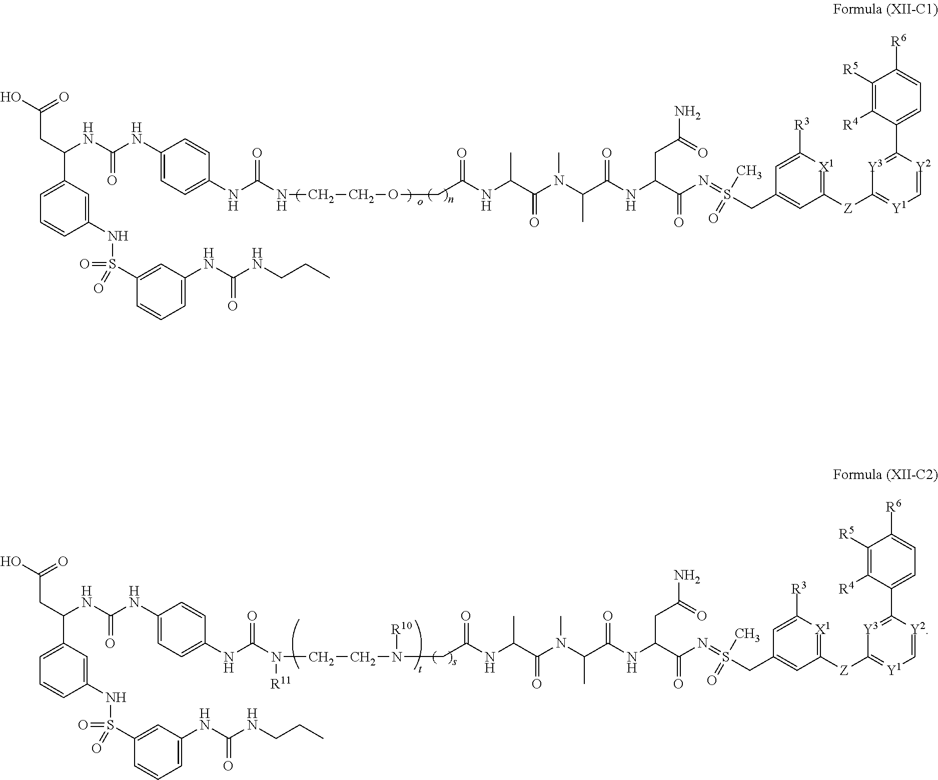

In some embodiments, provided herein is a compound, or a pharmaceutically acceptable salt thereof; or a stereoisomer or mixture of stereoisomers thereof, having a structure of Formula (XII-C1) or Formula (XII-C2), wherein:
X¹ is CH or N;
Y² is CF or N;
Y³ is CH or N;
R³ is hydrogen;
R⁴ is —OCH₃; or
R³ and R⁴ are taken together to form —O—C₂₋₁₀ alkyl-O— or —O—C₂₋₁₀ alkyl-NH—;
R⁵ is hydrogen; or
R³ and R⁵ are taken together to form —O—C₂₋₁₀ alkyl-O— or —O—C₂₋₁₀ alkyl-NH—;
R¹⁰ is —CH₃;
R¹¹ is hydrogen or —CH₃;
n is 2 to 4;
o is 2 to 4;
s is 1 to 4; and
t is 2 to 4.

In some embodiments, provided herein is a compound that is:

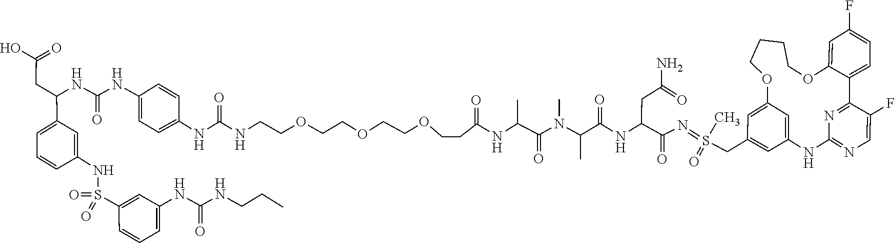 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(C)C(=O)N(C)C(C)C(=O)NC(CC(N)=O)C(=O)N=S(C)(=O)Cc4cc5cc(c4)OCCCCOc4cc(F)ccc4-c4nc(ncc4F)N5)cc3)c2)c1]

or a pharmaceutically acceptable salt thereof; or a stereoisomer or mixture of stereoisomers thereof.
Spacers (L¹, L², L³, L⁴, L⁵, L⁶, and L⁷)

Provided herein are compounds having linkers or spacers (used interchangeably) which serve to create physical space between one or more elements of the compound or conjugate. In some embodiments, the linkers or spacers provide additional utility (e.g., functional linkers). For example, in some embodiments, a linker is provided with a particular length that enables enhanced cleavage of an adjacent enzymatically cleavable moiety (e.g., EL). In some embodiments, a linker is provided with a particular length that reduces steric hinderance and/or enables greater target binding. In some embodiments, a linker is provided in a particular length such that an integrin binder can bind an integrin receptor (e.g., an αᵥβ₃ integrin receptor) with potency (i.e., IC₅₀) that is 1.0⁻⁹ M or lower (e.g., 9E⁻¹⁰, 8E⁻¹⁰, 7E⁻¹⁰, 6E⁻¹⁰, 5E⁻¹⁰, 4E⁻¹⁰, 3E⁻¹⁰, 2E⁻¹⁰, 1E⁻¹⁰, or lower). In some embodiments, a linker is provided in a particular length such that an integrin binder can bind an integrin receptor (e.g., an αᵥβ₃ integrin receptor) with potency (i.e., IC₅₀) that is 1.0⁻¹⁰ M or lower (e.g., 9E⁻¹¹, 8E⁻¹¹, 7E⁻¹¹, 6E⁻¹¹, 5E⁻¹¹, 4E⁻¹¹, 3E⁻¹¹, 2E⁻¹¹, 1E⁻¹¹, or lower). In some embodiments, a linker is provided in a particular length such that an integrin binder can bind an integrin receptor (e.g., an αᵥβ₃ integrin receptor) with potency (i.e., IC₅₀) that is 1.0⁻¹¹ M or lower (e.g., 9E⁻¹², 8E⁻¹², 7E⁻¹², 6E⁻¹², 5E⁻¹², 4E⁻¹², 3E⁻¹², 2E⁻¹², E⁻¹², or lower).

In some embodiments, provided herein is a compound comprising a linker (e.g., L¹, L², L³, L⁴, L⁵, L⁶, and/or L⁷), wherein the linker enhances retention of the compound within a tumor microenvironment. In some embodiments, a linker disclosed herein (e.g., a polyamine or polyamide linker) increases the tumor to plasma ratio of a compound, compared to a reference compound comprising an alkyl or PEG linker.

In some embodiments, a linker is a branched or linear chain of atoms selected from C, N, O, or S (each of which being substituted with hydrogens or bonds so as to fulfill standard valence). As used herein, oxides, e.g., C(O), N-oxides, S(O), S(O)₂, etc.) are considered to be substituted forms of C, N, and S respectively. In some embodiments, a linker is a carbonyl (—C(O)—). In some embodiments, a linker contains six or less (non-hydrogen) atoms. In some embodiments, a linker contains about 10 to about 20 (non-hydrogen) atoms. In some embodiments, a linker contains about 10 to about 20 (non-hydrogen) atoms arranged in a linear chain. In some embodiments, a linker contains about 20 to about 30 (non-hydrogen) atoms. In some embodiments, a linker contains about 20 to about 30 (non-hydrogen) atoms arranged in a linear chain. In some embodiments, a linker contains about 30 to about 40 (non-hydrogen) atoms. In some embodiments, a linker contains about 30 to about 40 (non-hydrogen) atoms arranged in a linear chain. In some embodiments, multiple linkers are present, each of which having a different length. In some embodiments, a linker contains cyclic or branched moieties. In some embodiments, a linker is substituted with one or more alkyl, oxo, amino, or amide groups.

In some embodiments, each of L¹, L², L³, L⁵, L⁶, and L⁷ contains or is terminally substituted with one or more carbonyl groups (—C(O)—), amine groups (e.g., —NH— or —N(CH₃)—), or amide groups (e.g., —C(O)NH—, —C(O)N(CH₃)—, —NHC(O)—, or N(CH₃)C(O)—).

In some embodiments, each of L¹, L², L³, L⁵, L⁶, and L⁷ is a substituted or unsubstituted C₂₋₂₀ alkyl chain that is optionally interrupted one or more times by groups selected from —C(O)—, —C(O)NH—, —C(O)N(CH₃)—, —C(O)O—, —NH—, —NHC(O)—, —N(CH₃)—, —N(CH₃)C(O)—, —NHC(O)NH—, —N(CH₃)C(O)NH—, —O—, —S—, —S(O)—, —S(O)₂—, —S(O)₂NH—, —NHS(O)₂NH—, —NHS(O)₂NHC(O)—, —NHSO₂NHC(O)O—, or any combination thereof.

In some embodiments, L¹, L², L³, L⁴, L⁵, L⁶, and L⁷ is substituted with one or more side-chain groups (e.g., alkylamines, alkylacids, alkylamides, alkylalcohols, etc.). In some embodiments, L¹, L², L³, L⁴, L⁵, L⁶, and L⁷ is substituted with one or more side-chains comprising a carboxylic acid and/or an amine.

In some embodiments, a linker is ionized in a biological system (e.g., within an acidic tumor microenvironment). In some embodiments, an ionized or partially charged (e.g., partially positive or partially negative) moiety or moieties within a linker enhance localization of the compound in a preferred locale (e.g., extracellularly, within a tumor microenvironment).

In some embodiments, a linker (e.g., one or more of L¹, L², L³, L⁵, L⁶, and L⁷) is an optionally substituted polyamine or polyamide linker.

In some embodiments, a polyamine or polyamide linker is a functional linker.

In some embodiments, the polyamine linker of one or more of L¹, L², L³, L⁵, L⁶, and L⁷ forms an aminium ion (or optionally multiple aminium ions) in an acidic tumor microenvironment.

In some embodiments, the polyamine linker of one or more of L¹, L², L³, L⁵, L⁶, and L⁷ is selectively retained in a tumor microenvironment.

In some embodiments, each of L¹, L², L³, L⁴, L⁵, L⁶ and L⁷ is independently a bond, substituted or unsubstituted C₁₋₃₀ alkyl, or substituted or unsubstituted heteroalkyl. In some embodiments, each L¹, L², L³, L⁴, L⁵, L⁶ and L⁷ is independently —C(O)—, substituted or unsubstituted C₂₋₃₀ alkyl, or substituted or unsubstituted heteroalkyl.

In some embodiments, each of L¹, L², L³, L⁴, L⁵, L⁶ and L⁷ is a non-cleavable linker. In some embodiments, each of L¹, L², L³, L⁴, L⁵, L⁶ and L⁷ is a non-cleavable heteroalkyl linker, optionally comprising one or more (e.g., 1 to 10) polymeric subunits (e.g., PEG or PEI). In some embodiments, each of L¹, L², L³, L⁴, L⁵, L⁶ and L⁷ is a non-cleavable polymeric linker. In some embodiments, each of L¹, L², and L³ is a non-cleavable polymeric linker.

In some embodiments, each of L¹, L², L³, L⁴, L⁵, L⁶ and L⁷ is a substituted or unsubstituted C₂₋₂₀ alkyl chain that is optionally interrupted one or more times by groups, each independently selected from —O—, —S—, —NH—, —N(CH₃)—, —C(O)—, —C(O)NH—, —C(O)N(CH₃)—, —C(O)O—, —NHC(O)—, N(CH₃)C(O)—, or —NHC(O)NH—, or any combination thereof.

Preference is given to embodiments wherein each of L¹, L², L³, L⁴, L⁵, L⁶ and L⁷, contains (e.g., is terminally substituted with) one or more carbonyl groups (—C(O)—), amine groups (e.g., —NH— or —N(CH₃)—), or amide groups (e.g., —C(O)NH—, —C(O)N(CH₃)—, —NHC(O)—, or N(CH₃)C(O)—). In some embodiments, spacers such as L¹, L², L³, L⁴, and L⁶ contain one or more polymeric units such as:

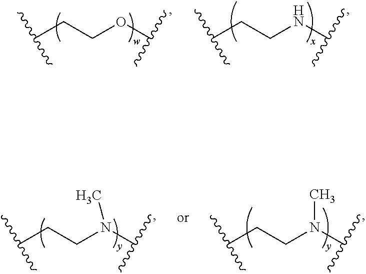

wherein w, x, and y are integers between 0 and 20, preferably between 1 and 10, more preferably between 2 and 6. In some embodiments, w, x, and y are each 2 or 3.

In some embodiments, one or more of L¹, L², L³, L⁴, L⁵, L⁶ and L⁷ is an optionally substituted polyamine linker. In some embodiments, a polyamine linker of L¹, L², L³, L⁴, L⁵, L⁶ and/or L⁷ forms an aminium ion within an acidic tumor microenvironment. In some embodiments, a polyamine linker of L¹, L², L³, L⁴, L⁵, L⁶ and/or L⁷ is retained in a tumor microenvironment. In some embodiments, a polyamine linker of L¹, L², L³, L⁴, L⁵, L⁶ and/or L⁷ enhances retention of a conjugate in a tumor microenvironment. In some embodiments, a polyether linker of L¹, L², L³, L⁵, L⁶ and/or L⁷ enhances the aqueous solubility of a conjugate and/or reduces aggregation. In some embodiments, an extended linker (e.g., L⁷) enhances target binding and/or release of the active agent (e.g., PT) by reducing steric hinderance and/or optimally positioning the integrin binder and PTEFb inhibitor.

In some embodiments, a polyamide linker of L¹, L², L³, L⁴, L⁵, L⁶, and/or L⁷, (e.g., a polysarcosine linker) increases the tumor to plasma ratio of the compound comprising said linker, compared to a reference compound comprising an analogous alkyl or PEG linker.

In some embodiments, L¹, L², L³, L⁴, L⁵ and/or L⁶ is a simple spacer selected from the group consisting of:
—O— —S—
—S(O)₂— —C(O)—
—C₁₋₃₀ alkyl-, —C(O)—C₁₋₃₀ alkyl
— —C₁₋₃₀ alkyl-C(O)—, —C(O)—C₁₋₃₀ alkyl-C(O)—,
—C₁₋₃₀ alkyl-C(O)NH—, —C(O)—C₁₋₃₀ alkyl-C(O)NH—,
—C₁₋₃₀ alkyl-C(O)N(CH₃)—, —C(O)—C₁₋₃₀ alkyl-C(O)N(CH₃)—,
—C₁₋₃₀ alkyl-NH—, —C(O)—C₁₋₃₀ alkyl-NH—,
—C₁₋₃₀ alkyl-NHC(O)—, —C(O)—C₁₋₃₀ alkyl-NHC(O)—,
—C₁₋₃₀ alkyl-N(CH₃)—, —C(O)—C₁₋₃₀ alkyl-N(CH₃)—,
—C₁₋₃₀ alkyl-N(CH₃)C(O)—, —C(O)—C₁₋₃₀ alkyl-N(CH₃)C(O)—,
—C(O)NH—, —C(O)N(CH₃)—
—C(O)NH—C₁₋₃₀ alkyl-, —C(O)N(CH₃)—C₁₋₃₀ alkyl
—C(O)NH—C₁₋₃₀ alkyl-C(O)—, —C(O)N(CH₃)—C₁₋₃₀ alkyl-C(O)—,
—C(O)NH—C₁₋₃₀ alkyl-C(O)NH—, —C(O)N(CH₃)—C₁₋₃₀ alkyl-C(O)NH—,
—C(O)NH—C₁₋₃₀ alkyl-C(O)N(CH₃)—, —C(O)N(CH₃)—C₁₋₃₀ alkyl-C(O)N(CH₃)—,
—C(O)NH—C₁₋₃₀ alkyl-NH—, —C(O)N(CH₃)—C₁₋₃₀ alkyl-NH—,
—C(O)NH—C₁₋₃₀ alkyl-NHC(O)—, —C(O)N(CH₃)—C₁₋₃₀ alkyl-NHC(O)—,
—C(O)NH—C₁₋₃₀ alkyl-N(CH₃)—, —C(O)N(CH₃)—C₁₋₃₀ alkyl-N(CH₃)—,
—C(O)NH—C₁₋₃₀ alkyl-N(CH₃)C(O)—, —C(O)N(CH₃)—C₁₋₃₀ alkyl-N(CH₃)C(O)—,
—NH—, —NHC(O)—,
—NH—C₁₋₃₀ alkyl-, —NHC(O)—C₁₋₃₀ alkyl-,
—NH—C₁₋₃₀ alkyl-C(O)—, —NHC(O)—C₁₋₃₀ alkyl-C(O)—,
—NH—C₁₋₃₀ alkyl-C(O)NH—, —NHC(O)—C₁₋₃₀ alkyl-C(O)NH—,
—NH—C₁₋₃₀ alkyl-C(O)N(CH₃)—, —NHC(O)—C₁₋₃₀ alkyl-C(O)N(CH₃)—,
—NH—C₁₋₃₀ alkyl-NH—, —NHC(O)—C₁₋₃₀ alkyl-NH—,
—NH—C₁₋₃₀ alkyl-NHC(O)—, —NHC(O)—C₁₋₃₀ alkyl-NHC(O)—,
—NH—C₁₋₃₀ alkyl-N(CH₃)—, —NHC(O)—C₁₋₃₀ alkyl-N(CH₃)—,
—NH—C₁₋₃₀ alkyl-N(CH₃)C(O)—, —NHC(O)—C₁₋₃₀ alkyl-N(CH₃C(O)—,
—N(CH₃)—, —N(CH₃)C(O)—,
—N(CH₃)—C₁₋₃₀ alkyl-, —N(CH₃)C(O)—C₁₋₃₀ alkyl-,
—N(CH₃)—C₁₋₃₀ alkyl-C(O)—, —N(CH₃)C(O)—C₁₋₃₀ alkyl-C(O)—,
—N(CH₃)—C₁₋₃₀ alkyl-C(O)NH—, —N(CH₃)C(O)—C₁₋₃₀ alkyl-C(O)NH—,
—N(CH₃)—C₁₋₃₀ alkyl-C(O)N(CH₃)—, —N(CH₃)C(O)—C₁₋₃₀ alkyl-C(O)N(CH₃)—,
—N(CH₃)—C₁₋₃₀ alkyl-NH—, —N(CH₃)C(O)—C₁₋₃₀ alkyl-NH—,
—N(CH₃)—C₁₋₃₀ alkyl-NHC(O)—, —N(CH₃)C(O)—C₁₋₃₀ alkyl-NHC(O)—,
—N(CH₃)—C₁₋₃₀ alkyl-N(CH₃)—, —N(CH₃)C(O)—C₁₋₃₀ alkyl-N(CH₃)—,
—N(CH₃)—C₁₋₃₀ alkyl-N(CH₃)C(O)—, and —N(CH₃)C(O)—C₁₋₃₀ alkyl-N(CH₃C(O)—.

In some embodiments, L¹, L², L³, L⁴, L⁵, L⁶, and/or L⁷ is a compound linker comprising two or more (e.g., one, two, three, or four) simple spacer elements defined above. In some embodiments, the two or more elements of the compound linker are conjoined by an interrupting group (e.g., a sulfamide group (e.g., —NHS(O)₂NH—, —NHS(O)₂NHC(O)—, or —NHS(O)₂NHC(O)O—), or an aryl, heteroaryl or heteroaralkyl (e.g., a substituted triazole, a substituted DBCO, or a combination or derivative thereof). In some embodiments, the compound linker further comprises one or more units selected from the group consisting of:

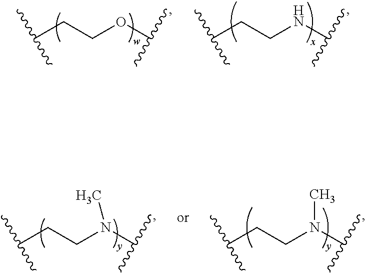

(e.g., wherein y is 1 to 20).

In some embodiments, L¹, L², L³, L⁴, L⁵, L⁶ and/or L⁷ is a combination of two or three simple spacers. In some embodiments, L¹, L², L³, L⁴, L⁵, L⁶ and/or L⁷ is a combination of two or three simple spacers, interrupted by (e.g., conjoined by) an amino acid, a dipeptide, or a tripeptide. In some embodiments, L¹, L², L³, L⁴, L⁵, L⁶ and/or L⁷ is a combination of two or three simple spacers, interrupted by (e.g., conjoined by) a cycloalkyl, heterocycloalkyl, aryl, or heteroaryl. In some embodiments, the interrupting group is a substituted triazole. In some embodiments, the interrupting group is an amino acid. In some embodiments, the interrupting group is: —S—, —S(O)—, —S(O)₂—,

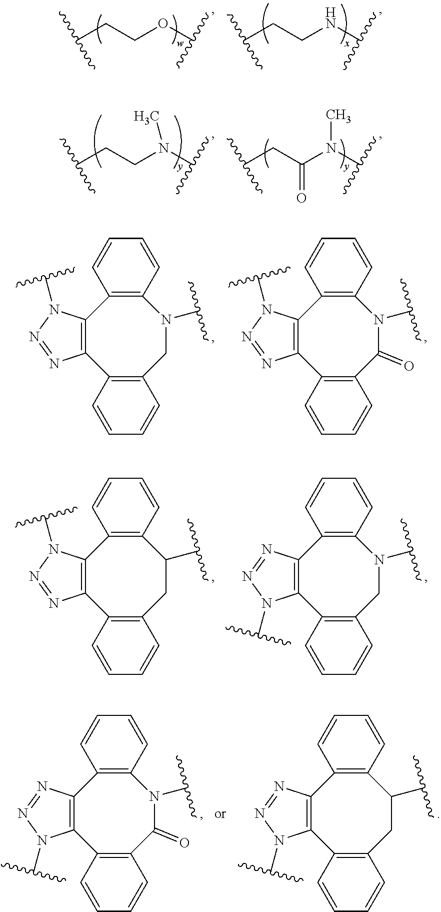

In some embodiments, L¹, L², L³, L⁴, L⁵, L⁶, and/or L⁷ is a substituted or unsubstituted C₂₋₂₀ alkyl chain that is optionally interrupted one or more times by groups selected from —C(O)—, —C(O)NH—, —C(O)N(CH₃)—, —C(O)O—, —NH—, —N(CH₃)—, —NHC(O)—, —N(CH₃)C(O)—, —NHC(O)NH—, —O—, —S—, —S(O)—, —S(O)₂—, carbocyclyl, heterocyclyl, aralkyl, heteroaralkyl, or any combination thereof. In some embodiments, L¹, L², L³, L⁴, L⁵, L⁶, and/or L⁷ is a linker disclosed in WO2016207089, which is incorporated by reference in its entirety. In some embodiments, a linker comprises two simple spacers which are conjoined via an aryl or heteroaryl group (e.g., a triazole, a dibenzocyclooctyne, or a derivative thereof). For example, a linker may comprise a dibenzylcyclooctyne (DBCO) derivative such as a DBCO NHS ester, or a chemical group formed therefrom. For example, an interrupting group may include:

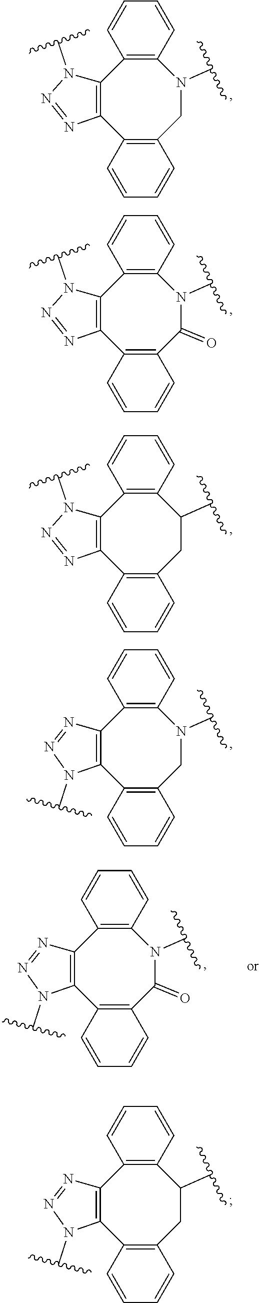

or a derivative thereof, wherein the interrupting group is substituted on each end to form a linker (e.g., L¹, L², L³, L⁴, L⁵, L⁶, and/or L⁷).

In some embodiments, L¹, L², L³, L⁴, L⁵ and/or L⁶ is a polymeric spacer having a structure represented by formula (i), (ii), or (iii) below:

—(CO)ₘ(CH₂)ₙ(OC₁₋₆ alkyl)ₒ(NH)ₚ(CO)_{q}—; (i)

—(CO)ᵣ(CH₂)ₛ(NR¹⁰C₁₋₆ alkyl)ₜ(NR¹¹)ᵤ(CO)ᵥ—; or (ii)

—(CO)ᵣ(CH₂)ₛ(NR¹⁰C(O)C₁₋₆ alkyl)ₜ(NR¹¹)ᵤ(CO)ᵥ—; (iii)
wherein:
R¹⁰ is, in each instance, independently selected from hydrogen or C₁₋₃ alkyl;
R¹¹ is, in each instance, independently selected from hydrogen or C₁₋₃ alkyl;
m is 0 or 1;
n is 0 to 10;
o is 1 to 10;
p is 0 or 1;
q is 0 or 1;
r is 0 or 1;
s is 0 to 10;
t is 1 to 10;
u is 0 or 1;
v is 0 or 1.

In some embodiments, L¹, L², L³, L⁴, L⁵, and/or L⁶ is a polymeric spacer selected from:
—C(O)—C₁₋₆ alkyl-[O—C₁₋₆ alkyl]₁₋₈-NHC(O)—,
—C(O)—C₁₋₆ alkyl-[O—C₁₋₆ alkyl]₁-s-N(CH₃)C(O)—,
—C(O)—C₁₋₆ alkyl-[NH—C₁₋₆ alkyl]₁₋₈-NHC(O)—,
—C(O)—C₁₋₆ alkyl-[NH—C₁₋₆ alkyl]1-s-N(CH₃)C(O)—,
—C(O)—C₁₋₆ alkyl-[N(CH₃)—C₁₋₆ alkyl]₁₋₈-NHC(O)—,
—C(O)—C₁₋₆ alkyl-[N(CH₃)—C₁₋₆ alkyl]1-s-N(CH₃)C(O)—,
—C(O)—C₁₋₆ alkyl-[N(CH₃)C(O)—C₁₋₆ alkyl]₁₋₈-NHC(O)—,
—C(O)—C₁₋₆ alkyl-[N(CH₃)C(O)—C₁₋₆ alkyl]₁₋₈-N(CH₃)C(O)—,
—C(O)—C₁₋₆ alkyl-[O—C₁₋₆ alkyl]₁₋₈-NH—,
—C(O)—C₁₋₆ alkyl-[O—C₁₋₆ alkyl]₁₋₈-N(CH₃)—,
—C(O)—C₁₋₆ alkyl-[NH—C₁₋₆ alkyl]₁₋₈-NH—,
—C(O)—C₁₋₆ alkyl-[NH—C₁₋₆ alkyl]₁₋₈-N(CH₃)—,
—C(O)—C₁₋₆ alkyl-[N(CH₃)—C₁₋₆ alkyl]₁₋₈-NH—,
—C(O)—C₁₋₆ alkyl-[N(CH₃)—C₁₋₆ alkyl]₁₋₈-N(CH₃)—,
—C(O)—C₁₋₆ alkyl-[N(CH₃)C(O)—C₁₋₆ alkyl]₁₋₈-NH—,
—C(O)—C₁₋₆ alkyl-[N(CH₃)C(O)—C₁₋₆ alkyl]₁₋₈-N(CH₃)—,
—C₁₋₆ alkyl-[O—C₁₋₆ alkyl]₁₋₈-NHC(O)—,
—C₁₋₆ alkyl-[O—C₁₋₆ alkyl]₁₋₈-N(CH₃)C(O)—,
—C₁₋₆ alkyl-[NH—C₁₋₆ alkyl]₁₋₈-NHC(O)—,
—C₁₋₆ alkyl-[NH—C₁₋₆ alkyl]₁₋₈-N(CH₃)C(O)—,
—C₁₋₆ alkyl-[N(CH₃)—C₁₋₆ alkyl]₁₋₈-NHC(O)—,
—C₁₋₆ alkyl-[N(CH₃)—C₁₋₆ alkyl]₁₋₈-N(CH₃)C(O)—,
—C₁₋₆ alkyl-[N(CH₃)C(O)—C₁₋₆ alkyl]₁₋₈-NHC(O)—,
—C₁₋₆ alkyl-[N(CH₃)C(O)—C₁₋₆ alkyl]1-s-N(CH₃)C(O)—,
—C₁₋₆ alkyl-[O—C₁₋₆ alkyl]₁₋₈-NH—,
—C₁₋₆ alkyl-[O—C₁₋₆ alkyl]₁₋₈-N(CH₃)—,
—C₁₋₆ alkyl-[NH—C₁₋₆ alkyl]₁₋₈-NH—,
—C₁₋₆ alkyl-[NH—C₁₋₆ alkyl]₁₋₈-N(CH₃)—,
—C₁₋₆ alkyl-[N(CH₃)—C₁₋₆ alkyl]₁₋₈-NH—,
—C₁₋₆ alkyl-[N(CH₃)—C₁₋₆ alkyl]₁₋₈-N(CH₃)—,
—C₁₋₆ alkyl-[N(CH₃)C(O)—C₁₋₆ alkyl]₁₋₈-NH—, and
—C₁₋₆ alkyl-[N(CH₃)C(O)—C₁₋₆ alkyl]₁₋₈-N(CH₃)—.

In some embodiments, a spacer (e.g., L⁷) is an elongated linker having a structure represented by formula (iv) or (v) below:

—(CO)ₐ—(C₀₋₆ alkyl)-(Z¹C₁₋₆ alkyl)_{b}—Z²—(C₀₋₆ alkyl)-(Z³C₁₋₆ alkyl)_{c}—(Z⁴)ₐ—(CO)ₑ—; or (iv)

—(CO)ₐ—(C₀₋₆ alkyl)-(Z¹C(O)C₁₋₆ alkyl)_{b}—Z²—; (v)
wherein:
Z¹ is in each occurrence independently —O—, —NH—, or —N(C₁₋₃ alkyl)-;
Z² is a bond, —C(O)—, —C(O)—(C₁₋₆ alkyl)-, —C(O)—(C₁₋₆ alkyl)-C(O)—, —C(O)—(C₁₋₆ alkyl)-C(O)NH—, —C(O)—(C₁₋₆ alkyl)-C(O)N(CH₃)—, —C(O)NH—, —C(O)NH—(C₁₋₆ alkyl)-C(O)NH—, —NH—, —N(C₁₋₆ alkyl)-, —N(C₁₋₆ alkyl)C(O)—, —NHC(O)—, —NHC(O)NH—, or —NHC(O)—(C₁₋₆ alkyl)-NHC(O)—;
Z³ is in each occurrence independently —O—, —NH—, or —N(C₁₋₃ alkyl)-;
Z⁴ is —O—, —NH—, or —N(C₁₋₃ alkyl)-;
a is 0 or 1;
b is 1 to 10;
c is 1 to 10;
d is 0 or 1; and
e is 0 or 1.

In some embodiments, a spacer (e.g., L⁷) is an elongated spacer selected from the group consisting of:
—C(O)—C₂₋₄ alkyl-[O—C₂₋₄ alkyl]2-4-NHC(O)—C₂₋₄ alkyl-[O—C₂₋₄ alkyl]₂₋₄-NHC(O)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-NHC(O)—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-NHC(O)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-NHC(O)—,
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NHC(O)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-NHC(O)—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-NHC(O)—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-NHC(O)—,
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-NHC(O)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-NHC(O)—,
—C(O)—C₂₋₄ alkyl-[O—C₂₋₄ alkyl]₂₋₄-NHC(O)—C₂₋₄ alkyl-[O—C₂₋₄ alkyl]₂₋₄-N(CH₃)C(O)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)C(O)—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-N(CH₃)C(O)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)C(O)—,
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-N(CH₃)C(O)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄—N(CH₃)C(O)—,
—C(O)—C₁₋₄ alkyl-[N(CH₃)C(O)—C₁₋₄ alkyl]₂₋₄-N(CH₃)C(O)—C₁₋₄ alkyl-[N(CH₃)C(O)—C₁₋₄ alkyl]₂₋₄—N(CH₃)C(O)—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-N(CH₃)C(O)—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-N(CH₃)C(O)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)C(O)—,
—C₂₋₄ alkyl-[O—C₂₋₄ alkyl]₂₋₄-NHC(O)—C₂₋₄ alkyl-[O—C₂₋₄ alkyl]₂₋₄-NHC(O)—,
—C₂₋₄ alkyl-[O—C₂₋₄ alkyl]₂₋₄-N(CH₃)C(O)—C₂₋₄ alkyl-[O—C₂₋₄ alkyl]₂₋₄-NHC(O)—,
—C₁₋₄ alkyl-[NH—C₂₋₄ alkyl]₂₋₄-NHC(O)—C₁₋₄ alkyl-[NH—C₂₋₄ alkyl]₂₋₄-NHC(O)—,
—C₁₋₄ alkyl-[NH—C₂₋₄ alkyl]₂₋₄-N(CH₃)C(O)—C₁₋₄ alkyl-[NH—C₂₋₄ alkyl]₂₋₄-NHC(O)—,
—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NHC(O)—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-NHC(O)—,
—C₂₋₄ alkyl-[O—C₂₋₄ alkyl]₂₋₄-NHC(O)—C₁₋₄ alkyl-[N(CH₃)—C₂₋₄ alkyl]₂₋₄-NHC(O)—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-NHC(O)—,
—C₁₋₄ alkyl-[N(CH₃)—C₂₋₄ alkyl]₂₋₄-NHC(O)—C₂₋₄ alkyl-[O—C₂₋₄ alkyl]₂₋₄-NHC(O)—,
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-NHC(O)—,
—C₂₋₄ alkyl-[O—C₂₋₄ alkyl]₂₋₄-NHC(O)—C₂₋₄ alkyl-[O—C₂₋₄ alkyl]₂₋₄-N(CH₃)C(O)—,
—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)C(O)—,
—C₁₋₄ alkyl-[NH—C₂₋₄ alkyl]₂₋₄-NHC(O)—C₁₋₄ alkyl-[NH—C₂₋₄ alkyl]₂₋₄-N(CH₃)C(O)—,
—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)C(O)—,
—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-N(CH₃)C(O)—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C₂₋₄ alkyl-[O—C₂₋₄ alkyl]₂₋₄-NHC(O)—C₁₋₄ alkyl-[N(CH₃)—C₂₋₄ alkyl]₂₋₄-N(CH₃)C(O)—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C₁₋₄ alkyl-[N(CH₃)—C₂₋₄ alkyl]₂₋₄-NHC(O)—C₂₋₄ alkyl-[O—C₂₋₄ alkyl]₂₋₄-N(CH₃)C(O)—,
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)C(O)—,
—C(O)—C₂₋₄ alkyl-[O—C₂₋₄ alkyl]₂₋₄-NHC(O)—C₂₋₄ alkyl-[O—C₂₋₄ alkyl]₂₋₄-NH—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-NH—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-NH—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-NH—,
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NH—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-NH—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-NH—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-NH—,
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-NH—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-NH—,
—C(O)—C₂₋₄ alkyl-[O—C₂₋₄ alkyl]₂₋₄-NHC(O)—C₂₋₄ alkyl-[O—C₂₋₄ alkyl]₂₋₄-N(CH₃)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-N(CH₃)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)—,
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-N(CH₃)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-N(CH₃)—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)—,
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-N(CH₃)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)—,
—C₂₋₄ alkyl-[O—C₂₋₄ alkyl]₂₋₄-NHC(O)—C₂₋₄ alkyl-[O—C₂₋₄ alkyl]₂₋₄-NH—,
—C₂₋₄ alkyl-[O—C₂₋₄ alkyl]₂₋₄-N(CH₃)C(O)—C₂₋₄ alkyl-[O—C₂₋₄ alkyl]₂₋₄-NH—,
—C₁₋₄ alkyl-[NH—C₂₋₄ alkyl]₂₋₄-NHC(O)—C₁₋₄ alkyl-[NH—C₂₋₄ alkyl]₂₋₄-NH—,
—C₁₋₄ alkyl-[NH—C₂₋₄ alkyl]₂₋₄-N(CH₃)C(O)—C₁₋₄ alkyl-[NH—C₂₋₄ alkyl]₂₋₄-NH—,
—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NH—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-NH—,
—C₂₋₄ alkyl-[O—C₂₋₄ alkyl]₂₋₄-NHC(O)—C₁₋₄ alkyl-[N(CH₃)—C₂₋₄ alkyl]₂₋₄-NH—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-NH—,
—C₁₋₄ alkyl-[N(CH₃)—C₂₋₄ alkyl]₂₋₄-NHC(O)—C₂₋₄ alkyl-[O—C₂₋₄ alkyl]₂₋₄-NH—,
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-NH—,
—C₂₋₄ alkyl-[O—C₂₋₄ alkyl]₂₋₄-NHC(O)—C₂₋₄ alkyl-[O—C₂₋₄ alkyl]₂₋₄-N(CH₃)—,
—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)—,
—C₁₋₄ alkyl-[NH—C₂₋₄ alkyl]₂₋₄-NHC(O)—C₁₋₄ alkyl-[NH—C₂₋₄ alkyl]₂₋₄-N(CH₃)—,
—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)—,
—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-N(CH₃)—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)—,
—C₂₋₄ alkyl-[O—C₂₋₄ alkyl]₂₋₄-NHC(O)—C₁₋₄ alkyl-[N(CH₃)—C₂₋₄ alkyl]₂₋₄-N(CH₃)—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)—,
—C₁₋₄ alkyl-[N(CH₃)—C₂₋₄ alkyl]₂₋₄-NHC(O)—C₂₋₄ alkyl-[O—C₂₋₄ alkyl]₂₋₄-N(CH₃)—, and
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)—.

In some embodiments, a spacer (e.g., L¹, L², L³, L⁴, L⁵, L⁶, or L⁷) further comprises a spanner, wherein the spanner is a linker that conjoins the spacer to an adjacent group (e.g., EL, A¹, IN, MOD). A spanner can be a substituted or unsubstituted alkyl, or a substituted or unsubstituted heteroalkyl linker, typically comprising about 1 to about 20 atoms selected from carbon, nitrogen, and oxygen. Preferably, a spanner is a short alkyl or heteroalkyl group having functional groups at its ends that are configured to form a stable bond (e.g., a peptide bond) with an adjacent group. Examples of spanners include groups such as:
#C(O)—C₁₋₆ alkyl-C(O)—, #C(O)NH—C₁₋₆ alkyl-C(O)—,
#C(O)N(CH₃)—C₁₋₆ alkyl-C(O)—, #NH—C₁₋₆ alkyl-C(O)—,
#NHC(O)—C₁₋₆ alkyl-C(O)—, #N(CH₃)—C₁₋₆ alkyl-C(O)—,
#N(CH₃)C(O)—C₂₋₆ alkyl-C(O)—, #C(O)—C₁₋₆ alkyl-C(O)NH—,
#C(O)NH—C₁₋₆ alkyl-C(O)NH—, #C(O)N(CH₃)—C₁₋₆ alkyl-C(O)NH—,
#NH—C₁₋₆ alkyl-C(O)NH—, #NHC(O)—C₁₋₆ alkyl-C(O)NH—,
#N(CH₃)—C₁₋₆ alkyl-C(O)NH—, #N(CH₃)C(O)—C₁₋₆ alkyl-C(O)NH—,
#C(O)—C₁₋₆ alkyl-C(O)N(CH₃)—, #C(O)NH—C₁₋₆ alkyl-C(O)N(CH₃)—,
#C(O)N(CH₃)—C₁₋₆ alkyl-C(O)N(CH₃)—, #NH—C₁₋₆ alkyl-C(O)N(CH₃)—,
#NHC(O)—C₁₋₆ alkyl-C(O)N(CH₃)—, #N(CH₃)—C₁₋₆ alkyl-C(O)N(CH₃)—,
#N(CH₃)C(O)—C₁₋₆ alkyl-C(O)N(CH₃)—, #C(O)—C₁₋₆ alkyl-NH—,
#C(O)NH—C₁₋₆ alkyl-NH—, #C(O)N(CH₃)—C₁₋₆ alkyl-NH—,
#NH—C₁₋₆ alkyl-NH—, #NHC(O)—C₁₋₆ alkyl-NH—,
#N(CH₃)—C₁₋₆ alkyl-NH—, #N(CH₃)C(O)—C₁₋₆ alkyl-NH—,
#C(O)—C₁₋₆ alkyl-NHC(O)—, #C(O)NH—C₁₋₆ alkyl-NHC(O)—,
#C(O)N(CH₃)—C₁₋₆ alkyl-NHC(O)—, #NH—C₁₋₆ alkyl-NHC(O)—,
#NHC(O)—C₁₋₆ alkyl-NHC(O)—, #N(CH₃)—C₁₋₆ alkyl-NHC(O)—,
#N(CH₃)C(O)—C₁₋₆ alkyl-NHC(O)—, #C(O)—C₁₋₆ alkyl-N(CH₃)—,
#C(O)NH—C₁₋₆ alkyl-N(CH₃)—, #C(O)N(CH₃)—C₁₋₆ alkyl-N(CH₃)—,
#NH—C₁₋₆ alkyl-N(CH₃)—, #NHC(O)—C₁₋₆ alkyl-N(CH₃)—,
#N(CH₃)—C₁₋₆ alkyl-N(CH₃)—, #N(CH₃)C(O)—C₁₋₆ alkyl-N(CH₃)—,
#C(O)—C₁₋₆ alkyl-N(CH₃)C(O)—, #C(O)NH—C₁₋₆ alkyl-N(CH₃)C(O)—,
#C(O)N(CH₃)—C₁₋₆ alkyl-N(CH₃)C(O)—, #NH—C₁₋₆ alkyl-N(CH₃)C(O)—,
#NHC(O)—C₁₋₆ alkyl-N(CH₃)C(O)—, #N(CH₃)—C₁₋₆ alkyl-N(CH₃)C(O)—,
#N(CH₃)C(O)—C₁₋₆ alkyl-N(CH₃)C(O)—, #C(O)—C₁₋₆ alkyl-,
#C(O)NH—C₁₋₆ alkyl-, #C(O)N(CH₃)—C₁₋₆ alkyl-,
#NH—C₁₋₆ alkyl-, #NHC(O)—C₁₋₆ alkyl-,
#N(CH₃)—C₁₋₆ alkyl-, #N(CH₃)C(O)—C₁₋₆ alkyl-;
#C₁₋₆ alkyl-C(O)—, #C₁₋₆ alkyl-C(O)NH—,
#C₁₋₆ alkyl-C(O)N(CH₃)—, #C₁₋₆ alkyl-NH—,
#C₁₋₆ alkyl-NHC(O)—, #C₁₋₆ alkyl-N(CH₃)—, or
#C₁₋₆ alkyl-N(CH₃)C(O)—,
wherein #denotes a bond to the spacer, and - denotes a bond to the adjacent group.

In some embodiments, a spanner is #C(O)—C₂₋₆ alkyl-C(O)— or #NH—C₂₋₆ alkyl-NH—. In some embodiments, a spanner is #C(O)(CH₂CH₂CH₂)C(O)— or #NH(CH₂CH₂)NH—. As used herein spanners are denoted S¹, S², S³, etc. corresponding to the linker to which they are attached. Alternatively, a spanner may be described as a member of A¹, in which case the S¹, S², S³, numbering still pertains to the linker to which A¹ is bonded. In some embodiments, a spanner is a bond. In some embodiments, a spanner is absent.

In some embodiments, L¹ is selected from:

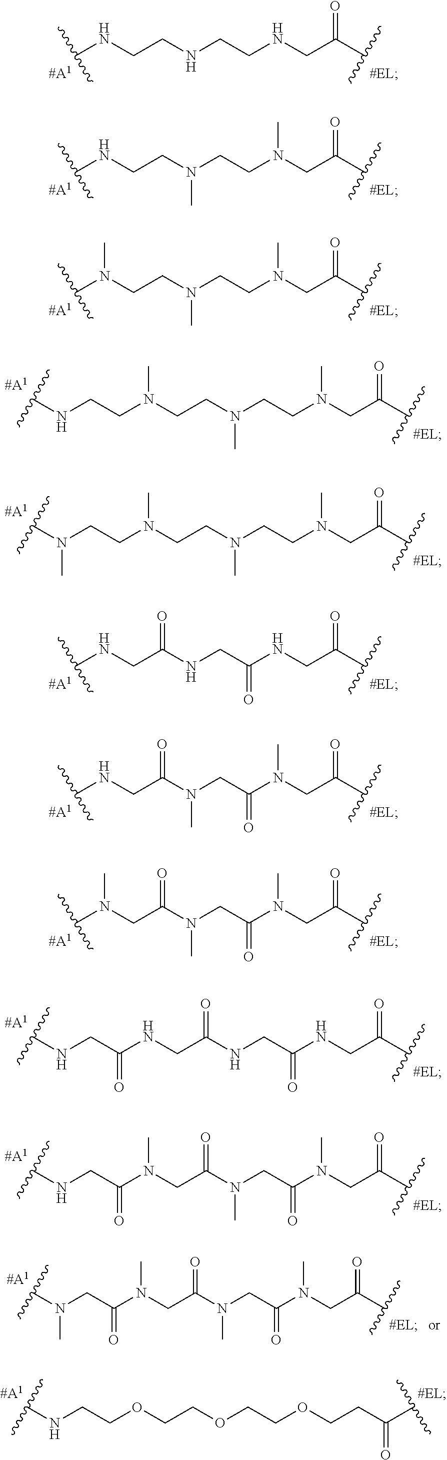

wherein #EL denotes a bond to EL (or SIL); and #A¹ denotes a bond to A¹.

In some embodiments, L² and L³ are each independently selected from:

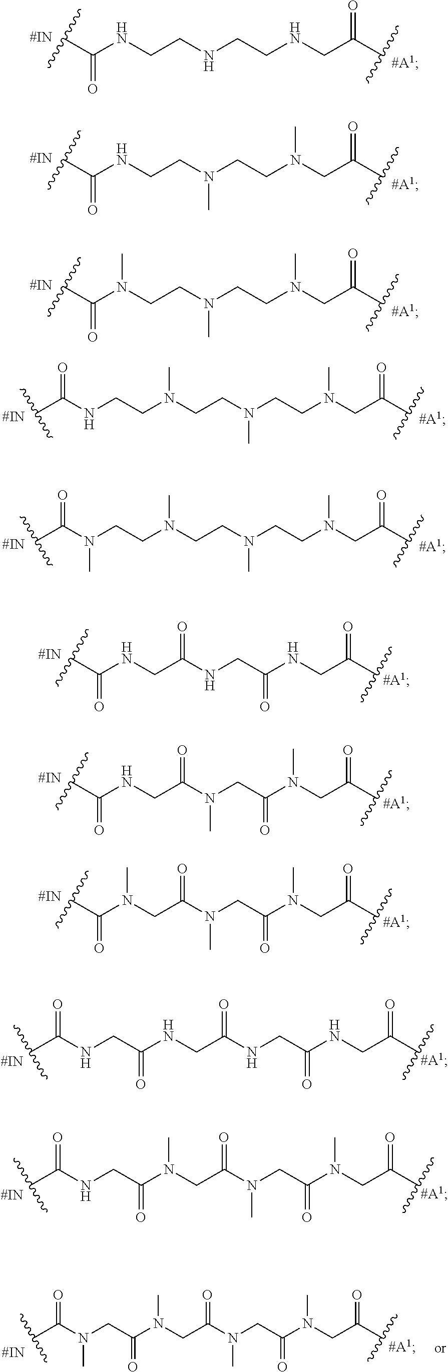

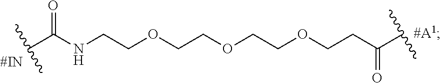 [SMILES: *C(=O)CCOCCOCCOCCNC(*)=O]

wherein #IN denotes a bond to IN or MOD; and #A¹ denotes a bond to A¹.

In some embodiments, L⁴ is:

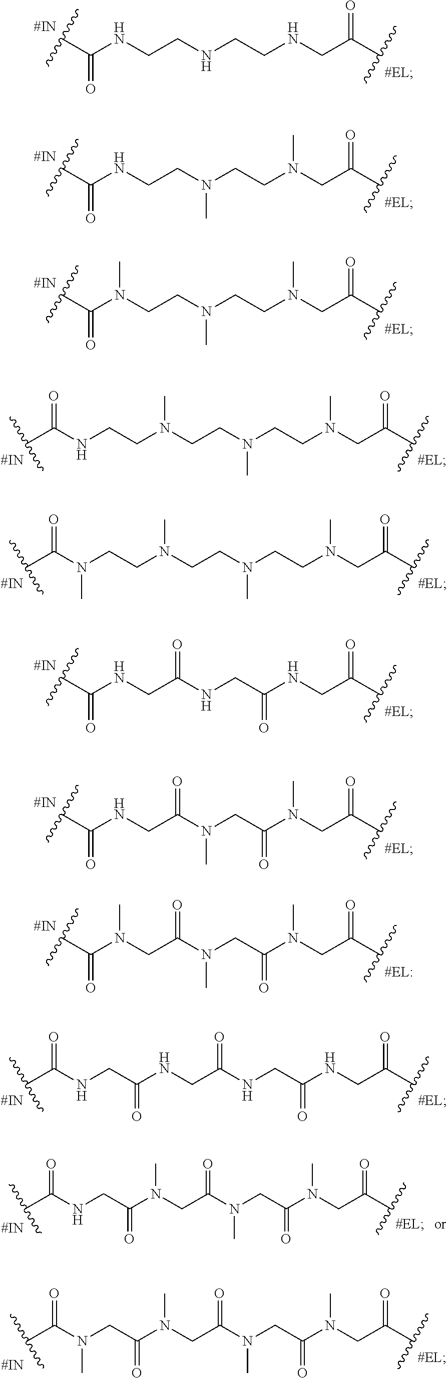

wherein #EL denotes a bond to EL (or SIL); and #IN denotes a bond to IN.

In some embodiments, L⁴ is:

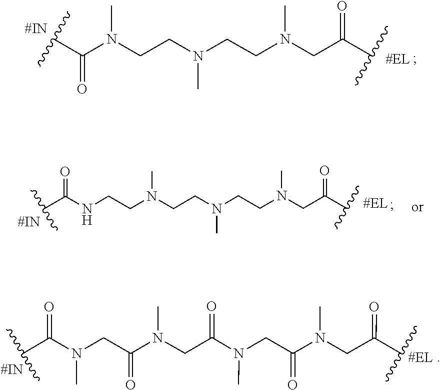 [SMILES: *C(=O)CN(C)CCN(C)CCN(C)C(*)=O ; *C(=O)CN(C)CCN(C)CCN(C)CCNC(*)=O ; *C(=O)CN(C)C(=O)CN(C)C(=O)CN(C)C(=O)CN(C)C(*)=O]

In some embodiments, L⁵ is:

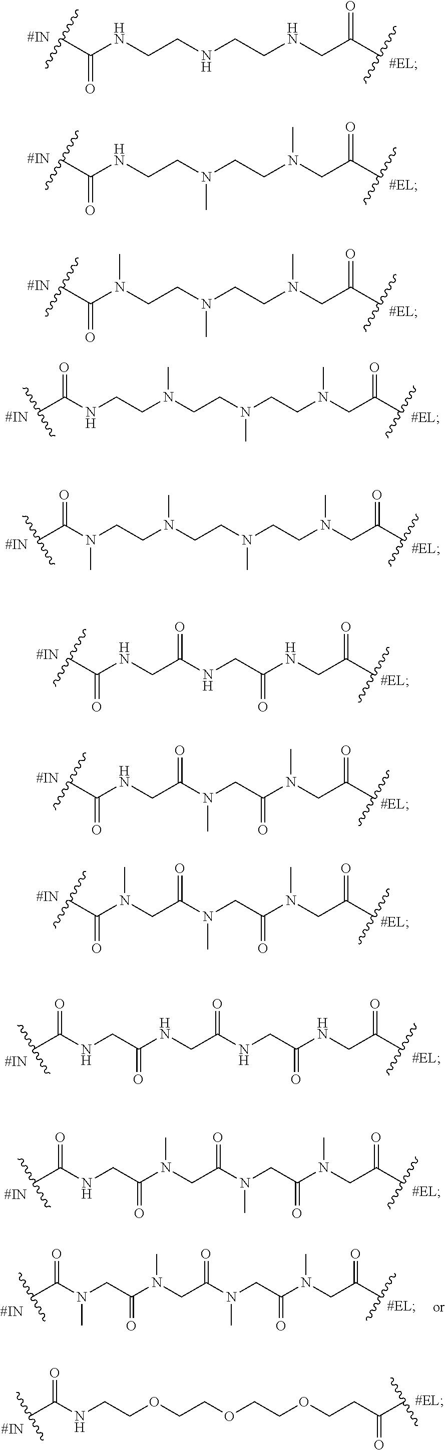

wherein #EL denotes a bond to EL (or SIL); and #IN denotes a bond to IN.

In some embodiments, L⁵ is:

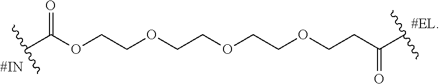 [SMILES: *C(=O)CCOCCOCCOCCOC(*)=O]

In some embodiments, L⁶ is: —C₀₋₁₂ alkyl-C(O)—; —C₀₋₁₂ alkyl-C(O)NH—;

—C₀₋₁₂ alkyl-C(O)N(CH₃)—; —C₀₋₁₂ alkyl-NH—; —C₀₋₁₂ alkyl-NHC(O)—;

—C₀₋₁₂ alkyl-N(CH₃)—; or —C₀₋₁₂ alkyl-N(CH₃)C(O)—;

In some embodiments, L⁶ is: —C(O)—.

In some embodiments, L⁷ is:

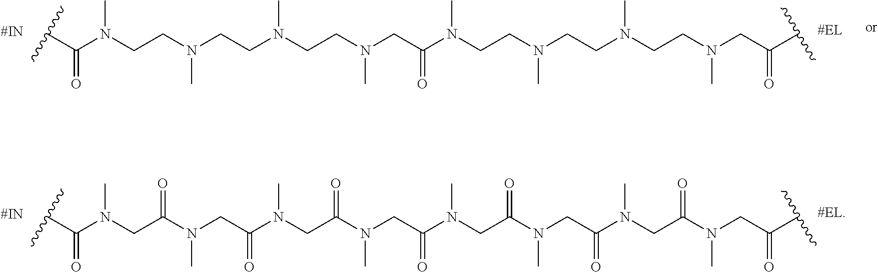 [SMILES: *C(=O)CN(C)CCN(C)CCN(C)CCN(C)C(=O)CN(C)CCN(C)CCN(C)CCN(C)C(*)=O ; *C(=O)CN(C)C(=O)CN(C)C(=O)CN(C)C(=O)CN(C)C(=O)CN(C)C(=O)CN(C)C(=O)CN(C)C(=O)CN(C)C(*)=O]

### Trivalent Radicals (A¹)

In some embodiments, a trivalent radical A¹ is a moiety containing 1 to 100 atoms selected from H, C, N, O, and S, configured to bond to linkers L¹, L², and L³. In some embodiments, A¹ contains a central atom or group (Y) that is N or CH. In some embodiments, the central atom or group (Y) has one or more arms (e.g., alkyl or heteroalkyl chains, optionally substituted with, or interrupted by, one or more groups independently selected from carbonyls (—C(O)—), ethers (—O—), amines (e.g., —NH— or —N(CH₃)—), or amides (e.g., —C(O)NH—, —C(O)N(CH₃)—, —NHC(O)—, or —N(CH₃)C(O)—, alternatively referred to as amide linkers). In some embodiments, A¹ is trivalent heteroalkyl radical. In some embodiments, A¹ is trivalent radical with amide linker arms (e.g., B¹, B², B³). In some embodiments, the trivalent radical is referred to as a branching unit, a branching group. In some embodiments, A of Formula (01) or Formula (05) is A¹. In some embodiments, A¹ is a trivalent heteroaryl, heteroaralkyl, aryl, aralkyl, heteroalkyl-aryl, heteroalkyl-heteroaryl radical. In some embodiments, A¹ contains a central atom or group (Y) that is a carbocycle (e.g., cycloalkyl or aryl) or heterocycle (e.g., heterocycloalkyl or heteroaryl). In some embodiments, (Y) is phenyl, triazinyl, or triazolyl, each of which is optionally substituted by 1-3 heteroalkyl arms.

In some embodiments, A or A¹ is a trivalent linker, optionally comprising arms or spanners, that is configured to conjoin a payload to two integrin binders, or to an integrin binder and a MOD group (e.g., via a spacer L and/or an enzymatic linker EL). In some embodiments, A or A¹ is a trivalent linker. In some embodiments, A or A¹ is a trivalent linker comprising arms B¹, B², and B³, and optionally further comprising spanners S¹, S², and S³. In some embodiments, each arm of the trivalent linker is configured to form an amide bond with an adjacent group (e.g., L¹, L², L, IN, MOD, or EL). In some embodiments, A or A¹ is a trivalent amide linker. In this context, a trivalent amide linker can be a chemical group having three valencies (or capable of forming bonds to 3 agents), and generally composed of alkyl, carbonyl, and amine groups, and preferably capable of forming an amide bond with an adjacent group (e.g., L¹, L², L³, IN, MOD, or EL). In some embodiments, the trivalent radical is a trivalent amide linker. In some embodiments, the trivalent amide linker comprises a central amino acid (e.g., lysine, glutamine, glutamate, etc.), wherein the amino acid forms a bond with an adjacent group (e.g., L¹, L², L³, IN, MOD, or EL), optionally bridged via a linking arm (e.g., B¹, B², B³) and/or a spanner (i.e., S¹, S² or S³). Preferably, a trivalent radical as described herein conjoins a payload (e.g., an enzymatically-cleavable payload (CT)) with two integrin binders, or with an integrin binder and a group MOD, and wherein the payload, integrin binder(s) and MOD may be conjoined via a linker/spacer (L¹, L², or L³).

In some embodiments, A¹ has a structure represented by the formula:

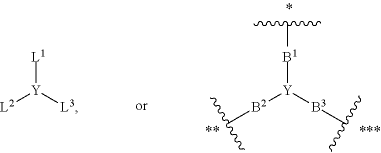

wherein (Y) is N, CH, phenyl, or heteroaryl, and each of B¹, B², and B³ represents an arm that connects to a corresponding spacer (e.g., L¹, L², and L³ respectively) and wherein * denotes a bond to L¹, ** denotes a bond to L², *** denotes a bond to L³.

In some embodiments, A¹ is a trivalent radical of the formula:

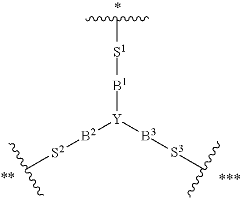

wherein * denotes a bond to L¹, ** denotes a bond to L², and *** denotes a bond to L³, each of S¹, S², S³, is an optionally present spanner (defined herein), each of B¹, B², B³, is an optionally present linking arm (defined herein), and Y is CH or N.

In some embodiments, one or more of S¹, S², and S³ is a bond. In some embodiments, one or more of S¹, S², and S³ is —C(O)—C₂₋₆ alkyl-C(O)—, —N(CH₃)—C₁₋₆ alkyl-N(CH₃)—, —NH—C₁₋₆ alkyl-NH—, or —NH—C₁₋₆ alkyl-N(CH₃)—. In some embodiments, S¹, S², and/or S³ is —C(O)—C₂₋₆ alkyl-C(O)— or —NH—C₁₋₆ alkyl-NH—.

In some embodiments, each B¹, B², and B³, is an amide linker (e.g., an alkyl amide and/or a polyamide linker). In some embodiments, each B¹, B², and B³, is an amide linker comprising 1 to about 32 atoms. In some embodiments, the 1 to 32 atoms are selected from carbon, nitrogen, and oxygen.

In some embodiments, each B¹, B², and B³, is selected from:
—C(O)—;
—C(O)NH—;
—C(O)N(CH₃)—;
—C(O)NH—C₀₋₆ alkyl-C(O)—;
—C(O)NH—C₀₋₆ alkyl-C(O)NH—;
—C(O)NH—C₀₋₆ alkyl-C(O)N(CH₃)—;
—C(O)NH—C₀₋₆ alkyl-NH—;
—C(O)NH—C₀₋₆ alkyl-N(CH₃)—;
—C(O)NH—C₀₋₆ alkyl-NHC(O)—;
—C(O)NH—C₀₋₆ alkyl-N(CH₃)C(O)—;
—C(O)N(CH₃)—C₀₋₆ alkyl-C(O)—;
—C(O)N(CH₃)—C₀₋₆ alkyl-C(O)NH—;
—C(O)N(CH₃)—C₀₋₆ alkyl-C(O)N(CH₃)—;
—C(O)NH—C₀₋₆ alkyl-NH—;
—C(O)NH—C₀₋₆ alkyl-N(CH₃)—;
—C(O)N(CH₃)—C₀₋₆ alkyl-NHC(O)—;
—C(O)N(CH₃)—C₀₋₆ alkyl-N(CH₃)C(O)—;
—C(O)—C₀₋₆ alkyl-;
—C(O)—C₀₋₆ alkyl-C(O)—;
—C(O)—C₀₋₆ alkyl-C(O)NH—;
—C(O)—C₀₋₆ alkyl-C(O)N(CH₃)—;
—C(O)—C₀₋₆ alkyl-NH—;
—C(O)—C₀₋₆ alkyl-N(CH₃)—;
—C(O)—C₀₋₆ alkyl-NHC(O)—;
—C(O)—C₀₋₆ alkyl-N(CH₃)C(O)—;
—C₀₋₆ alkyl-C(O)—;
—C₀₋₆ alkyl-C(O)NH—;
—C₀₋₆ alkyl-C(O)N(CH₃)—;
—C₀₋₆ alkyl-C(O)NH—C₀₋₆ alkyl-C(O)—;
—C₀₋₆ alkyl-C(O)NH—C₀₋₆ alkyl-C(O)NH—;
—C₀₋₆ alkyl-C(O)NH—C₀₋₆ alkyl-C(O)N(CH₃)—;
—C₀₋₆ alkyl-C(O)NH—C₀₋₆ alkyl-NH—;
—C₀₋₆ alkyl-C(O)NH—C₀₋₆ alkyl-N(CH₃)—;
—C₀₋₆ alkyl-C(O)NH—C₀₋₆ alkyl-NHC(O)—;
—C₀₋₆ alkyl-C(O)NH—C₀₋₆ alkyl-N(CH₃)C(O)—;
—C₀₋₆ alkyl-C(O)N(CH₃)—C₀₋₆ alkyl-C(O)—;
—C₀₋₆ alkyl-C(O)N(CH₃)—C₀₋₆ alkyl-C(O)NH—;
—C₀₋₆ alkyl-C(O)N(CH₃)—C₀₋₆ alkyl-C(O)N(CH₃)—;
—C₀₋₆ alkyl-C(O)NH—C₀₋₆ alkyl-NH—;
—C₀₋₆ alkyl-C(O)NH—C₀₋₆ alkyl-N(CH₃)—;
—C₀₋₆ alkyl-C(O)N(CH₃)—C₀₋₆ alkyl-NHC(O)—;
—C₀₋₆ alkyl-C(O)N(CH₃)—C₀₋₆ alkyl-N(CH₃)C(O)—;
—C₀₋₆ alkyl-C(O)—C₀₋₆ alkyl-C(O)—;
—C₀₋₆ alkyl-C(O)—C₀₋₆ alkyl-C(O)NH—;
—C₀₋₆ alkyl-C(O)—C₀₋₆ alkyl-C(O)N(CH₃)—;
—C₀₋₆ alkyl-C(O)—C₀₋₆ alkyl-NH—;
—C₀₋₆ alkyl-C(O)—C₀₋₆ alkyl-N(CH₃)—;
—C₀₋₆ alkyl-C(O)—C₀₋₆ alkyl-NHC(O)—;
—C₀₋₆ alkyl-C(O)—C₀₋₆ alkyl-N(CH₃)C(O)—;
—C₀₋₆ alkyl-NH—;
—C₀₋₆ alkyl-NHC(O)—;
—C₀₋₆ alkyl-NHC(O)—C₁₋₆ alkyl-C(O)—;
—C₀₋₆ alkyl-NHC(O)—C₁₋₆ alkyl-C(O)NH—;
—C₀₋₆ alkyl-NHC(O)—C₁₋₆ alkyl-C(O)N(CH₃)—;
—C₀₋₆ alkyl-NHC(O)—C₁₋₆ alkyl-NH—;
—C₀₋₆ alkyl-NHC(O)—C₁₋₆ alkyl-N(CH₃)—;
—C₀₋₆ alkyl-NHC(O)—C₁₋₆ alkyl-NHC(O)—;
—C₀₋₆ alkyl-NHC(O)—C₁₋₆ alkyl-N(CH₃)C(O)—;
—C₀₋₆ alkyl-N(CH₃)—;
—C₀₋₆ alkyl-N(CH₃)C(O)—;
—C₀₋₆ alkyl-N(CH₃)C(O)—C₁₋₆ alkyl-C(O)—;
—C₀₋₆ alkyl-N(CH₃)C(O)—C₁₋₆ alkyl-C(O)NH—;
—C₀₋₆ alkyl-N(CH₃)C(O)—C₁₋₆ alkyl-C(O)N(CH₃)—;
—C₀₋₆ alkyl-N(CH₃)C(O)—C₁₋₆ alkyl-NH—;
—C₀₋₆ alkyl-N(CH₃)C(O)—C₁₋₆ alkyl-N(CH₃)—;
—C₀₋₆ alkyl-N(CH₃)C(O)—C₁₋₆ alkyl-NHC(O)—; and
—C₀₋₆ alkyl-N(CH₃)C(O)—C₁₋₆ alkyl-N(CH₃)C(O)—.

In some embodiments, A¹ is an amino acid or a derivative thereof. In some embodiments, A¹ is a Lys (e.g., L-Lys or D-Lys), Glu (L-Glu or D-Glu), or Asp (L-Asp or D-Asp), or a derivative thereof (e.g., substituted with one or more arms (e.g., B¹, B², or B³)). In some embodiments, A¹ has one of the following structures:
*C(O)—C₀₋₆ alkyl-Y(C₀₋₆ alkyl-NH**)(C₀₋₆ alkyl-NH**),
*C(O)—C₀₋₆ alkyl-Y(C₀₋₆ alkyl-NH**)(C₀₋₆ alkyl-C(O)***),
*C(O)—C₀₋₆ alkyl-Y(C₀₋₆ alkyl-C(O)**)(C₀₋₆ alkyl-C(O)***),
*C(O)—C₀₋₆alkyl-C(O)NH—Y(C₀₋₆ alkyl-NH**)(C₀₋₆ alkyl-NH**),
*C(O)—C₀₋₆alkyl-C(O)NH—Y(C₀₋₆ alkyl-NH**)(C₀₋₆ alkyl-C(O)***),
*C(O)—C₀₋₆alkyl-C(O)NH—Y(C₀₋₆ alkyl-C(O)**)(C₀₋₆ alkyl-C(O)***),
*C(O)—C₀₋₆ alkyl-C(O)NH—Y(C₀₋₆ alkyl-C(O)NH—C₀₋₆ alkyl-NH**)(C₀₋₆ alkyl-C(O)NH—C₀₋₆ alkyl-NH***),
wherein each of *, **, and *** denotes a bond to a spacer L¹, L², or L³; and Y is N or CH. In some embodiments, Y is lysine, glutamine, or glutamic acid.

In some embodiments, A¹ has one of the following structures:

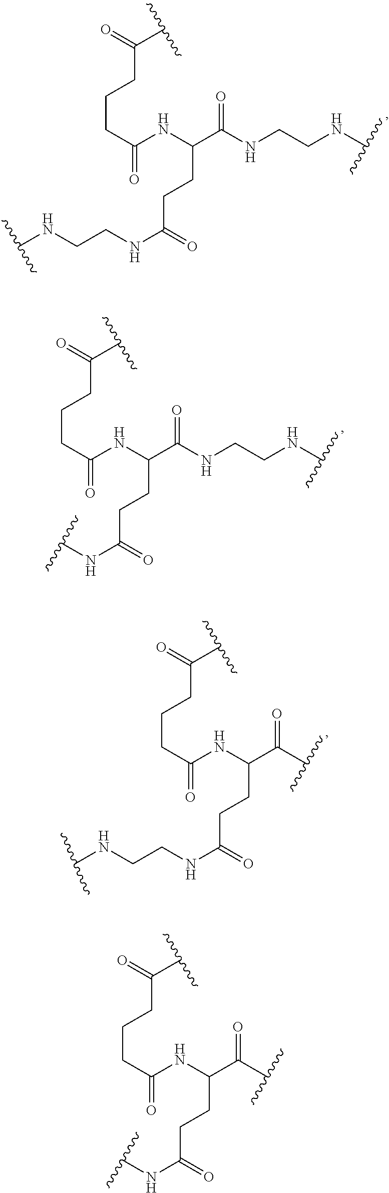 [SMILES: *NCCNC(=O)CCC(NC(=O)CCCC(*)=O)C(=O)NCCN* ; *NCCNC(=O)C(CCC(=O)N*)NC(=O)CCCC(*)=O ; *NCCNC(=O)CCC(NC(=O)CCCC(*)=O)C(*)=O ; *NC(=O)CCC(NC(=O)CCCC(*)=O)C(*)=O]

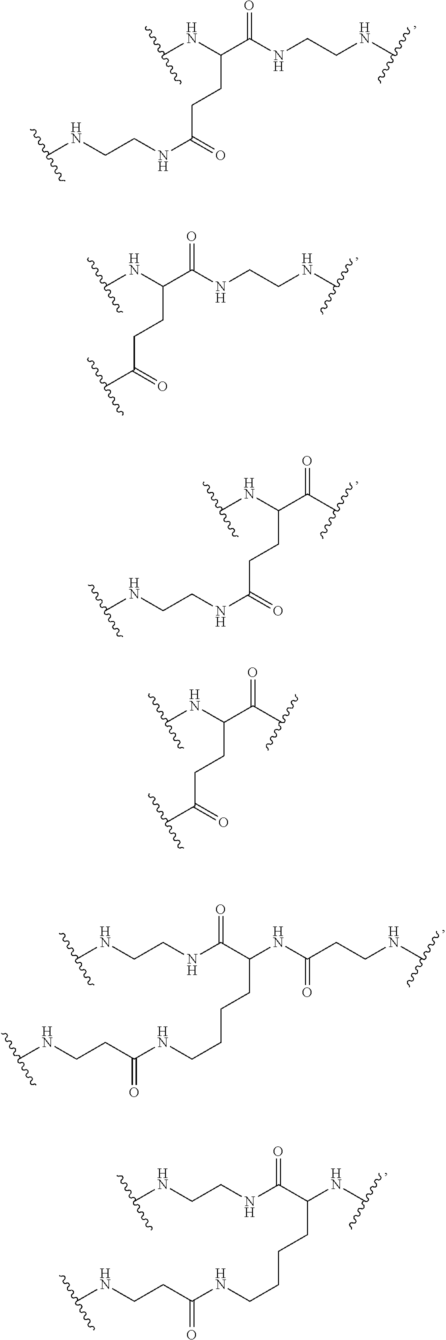 [SMILES: *NCCNC(=O)CCC(N*)C(=O)NCCN* ; *NCCNC(=O)C(CCC(*)=O)N* ; *NCCNC(=O)CCC(N*)C(*)=O ; *NC(CCC(*)=O)C(*)=O ; *NCCNC(=O)C(CCCCNC(=O)CCN*)NC(=O)CCN* ; *NCCNC(=O)C(CCCCNC(=O)CCN*)N*]

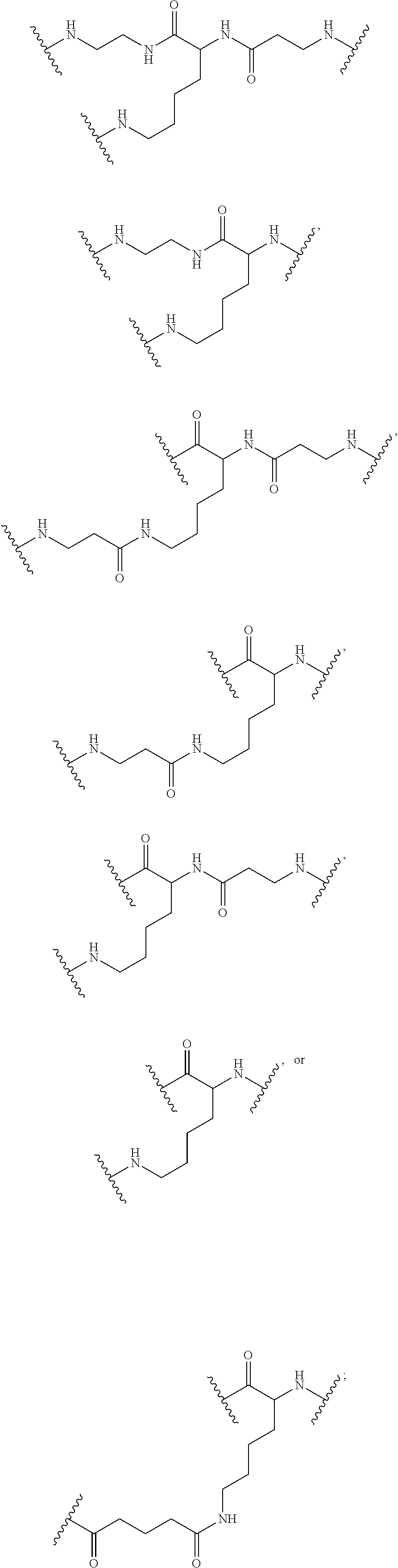

wherein each of *, **, and *** denotes a bond to a spacer L¹, L², or L³.

In some embodiments, each of L¹, L², and L³ is a non-cleavable linker and A¹ is an amino acid or a derivative thereof (e.g., substituted with one or more arms (e.g., B¹, B², or B³)).

In some embodiments, A¹ is of Formula (A¹-1a):

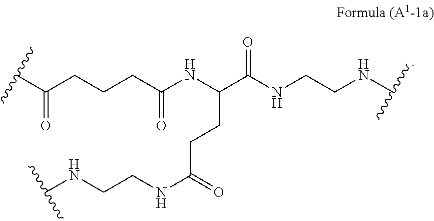 [SMILES: *NCCNC(=O)CCC(NC(=O)CCCC(*)=O)C(=O)NCCN*]

In some embodiments, A¹ is of Formula (A¹-1b):

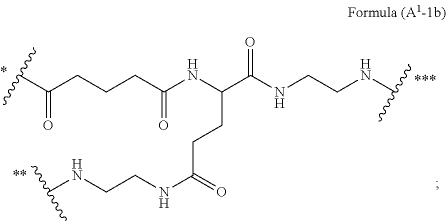 [SMILES: *NCCNC(=O)CCC(NC(=O)CCCC(*)=O)C(=O)NCCN*]

wherein * denotes a bond to L¹, ** denotes a bond to L², *** denotes a bond to L³.

In some embodiments, A¹ is of Formula (A¹-1c) or Formula (A¹-1d):

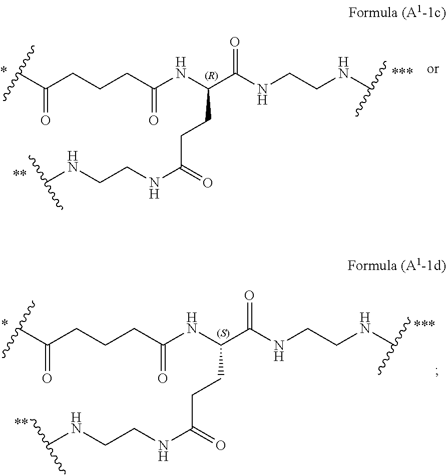

wherein * denotes a bond to L¹, ** denotes a bond to L², *** denotes a bond to L³.

In some embodiments, A¹ is of Formula (A¹-2b):

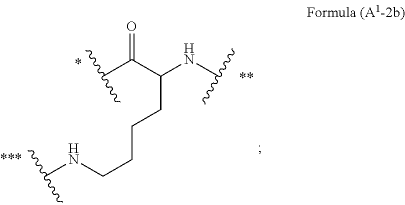 [SMILES: *NCCCCC(N*)C(*)=O]

wherein * denotes a bond to L¹, ** denotes a bond to L², *** denotes a bond to L³.

In some embodiments, A¹ is of Formula (A¹-2c) or Formula (A¹-2d):

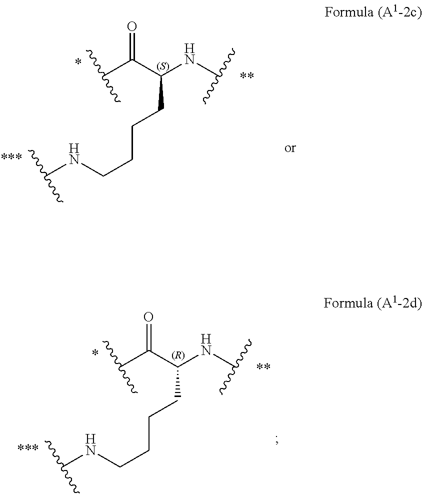

wherein * denotes a bond to L¹, ** denotes a bond to L², *** denotes a bond to L³.

In some embodiments, provided herein is a compound comprising a trivalent linker (A′) and two linker-binder groups (L²IN, L³IN), or a linker-binder and linker-MOD group (L²IN)(L³MOD), wherein the compound comprising the L³IN or L³MOD group has an increased half-life, compared to a compound comprising a bivalent linker. In some embodiments, provided herein is a compound comprising a trivalent linker (A¹) and two linker-binder groups (L²IN, L³IN), or a linker-binder and linker-MOD group (L²IN)(L³MOD), wherein the compound comprising the L³IN or L³MOD group has an increased AUC, compared to a compound comprising a bivalent linker. In some embodiments, provided herein is a compound comprising a trivalent linker (A′) and two linker-binder groups (L²IN, L³IN), or a linker-binder and linker-MOD group (L²IN)(L³MOD), wherein the compound comprising the L³IN or L³MOD group has decreased clearance and/or increased half-life, compared to a compound comprising a bivalent linker.

### Physicochemical or Pharmacokinetic Modulators (MOD)

In some embodiments, the present disclosure provides conjugates comprising a physicochemical or pharmacokinetic modulator (“MOD”). In some embodiments, MOD is a physicochemical modulator. In some embodiments, MOD is a pharmacokinetic modulator. Generally, these terms refer to the same groups and are used interchangeably herein, unless specified otherwise. A group MOD may be connected to the rest of the conjugate via a linker (e.g., a stable liner). In some embodiments, MOD is connected to EL via a linker (e.g., L⁶). In some embodiments, MOD is bonded to A¹ via a linker (e.g., L³). In some embodiments, MOD is a charged or a polar group. Examples of charged or polar groups include hydroxides and alcohols, carboxylates and carboxylic acids, aminiums and amines, guanidiniums and guanidines, and the like. In some embodiments, MOD is or comprises an amine. In some embodiments, MOD is a polyamine group. In some embodiments, MOD is —NR₂ or —NR₃⁺. In some embodiments, MOD is —C₁₋₆ alkyl-NR₂ or —C₁₋₆ alkyl-NR₃⁺ (e.g., —C₁₋₆ alkyl-NH₂, —C₁₋₆ alkyl-NHCH₃, —C₁₋₆ alkyl-N(CH₃)₂, or —C₁₋₆ alkyl-N(CH₃)₃⁺). In some embodiments, MOD is —C₁₋₆ alkyl-NHC(═NH⁺)NH₂. In some embodiments, MOD is a polyacid group. In some embodiments, MOD is —COOH or —COO⁻. In some embodiments, MOD is —C₁₋₆ alkyl-COOR (e.g., —C₁₋₆ alkyl-COOH, —C₁₋₆ alkyl-COO⁻, —C₁₋₆ alkyl-COO⁻Na⁺, or —C₁₋₆ alkyl-COOCH₃.). In some embodiments, MOD is a group that is converted to a polar or charged group in vivo (e.g., MOD is an alkyl ester, which is cleaved in vivo to an alkyl acid, thereby reducing membrane permeability). In some embodiments, MOD is —OH or —O⁻. In some embodiments, MOD is an amino acid. In some embodiments, MOD is a basic amino acid. In some embodiments, MOD is Arg, His, or Lys. In some embodiments, MOD is an acidic amino acid. In some embodiments, MOD is Glu or Asp. In some embodiments, MOD is an unnatural amino acid (e.g., D-Glu or D-Asp). In some embodiments, MOD is a polar amino acid (e.g., Ser, Thr, Asn, Gln). In some embodiments, provided herein is a compound of the formula:

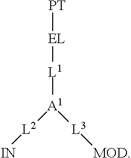

In some embodiments, L³ is a heteroalkyl linker. In some embodiments, L³ is a simple spacer. In some embodiments, L³ is a carbonyl, an acyl linker, an amide linker, or an alkyl linker. In some embodiments, L³ is an optionally substituted alkyl linker (e.g., an optionally substituted alkyl amide linker). In some embodiments, MOD is —OH, —COOH, —NH₂, NHCH₃, —N(CH₃)₂, —(CH₃)₃⁺, or a salt thereof. In some embodiments, L³ is C₁₋₁₂ alkyl. In some embodiments, L³ is NH₁₋₁₂ alkyl. In some embodiments, L³ is NHC(O)—₁₋₁₂ alkyl. In some embodiments, L³ is C(O)—C₁₋₁₂ alkyl. In some embodiments, L³ is a linker (L) or spacer (SP) as defined herein.

In some embodiments, L³ is —(CO)ᵣ(CH₂)ₛ(NR¹⁰C(O)C₁₋₆ alkyl)ₜ(NR¹¹)ᵤ(CO)ᵥ—. In some embodiments, L³ is —[N(CH₃)CH₂C(O)]₁₋₁₂ and MOD is —OH or —O⁻. Examples of L³-MOD include: —[N(CH₃)CH₂C(O)]₄O⁻, —[N(CH₃)CH₂C(O)]₆O⁻, and —[N(CH₃)CH₂C(O)]₈O⁻. In some embodiments, MOD is —OH, —COOH, —NH₂, NHCH₃, —N(CH₃)₂, —(CH₃)₃⁺, or a salt thereof. In some embodiments, L³ is C₁₋₁₂ alkyl. In some embodiments, L³ is NH₁₋₁₂ alkyl. In some embodiments, L³ is NHC(O)—₁₋₁₂ alkyl. In some embodiments, L³ is C(O)—C₁₋₁₂ alkyl. In some embodiments, L³ is as described herein.

### Enzymatically-Cleavable Linker (EL)

In some embodiments, the current invention relates to conjugates in which a peptide moiety is linked to a sulfoximine moiety present in CDK9 inhibitors. Substrate peptides of tumor stromal enzymes, such as, neutrophil elastase, in position P1-P3 and the sulfoximine moiety of the CDK9 inhibitor molecule in P1′ may be cleaved by such enzymes. While the aᵥβ₃-linker-CDK9 conjugates show only weak cytotoxic activity, the cytotoxic activity may be increased significantly in the presence of tumor-associated enzymes, such as, neutrophil elastase.

In an aspect, provided herein is a compound, or a pharmaceutically acceptable salt thereof; or a stereoisomer or mixture of stereoisomers thereof, having a structure represented by any one of Formulae (01)-(05):

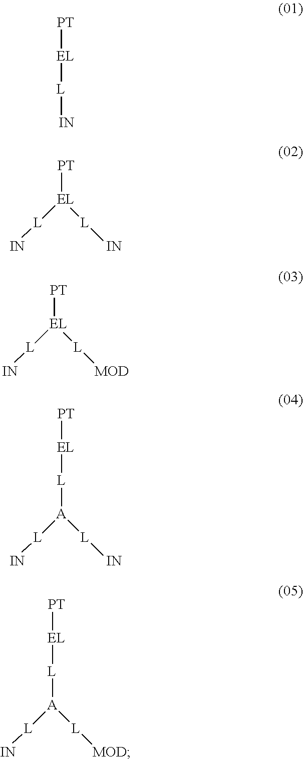

wherein:
PT is a PTEFb inhibitor group;
EL is an enzymatically-cleavable linker;
L is a stable linker;
A is a branching group;
IN is an integrin binding group; and
MOD is a physicochemical or pharmacokinetic modulator group.

In some embodiments, EL is a dipeptide having the formula -AA¹-AA²-, or a tripeptide having the formula AA¹-AA²-AA³-, wherein each AA¹, AA², and AA³ is independently an amino acid, or a derivative thereof. In some embodiments, EL further comprises a self-immolative linker (SIL).

In some embodiments, provided herein is a compound of any one of Formulae (01)-(05), wherein EL has the formula:

*-AA¹-AA²-(AA³)₀₋₁-(SIL)₀₋₁-**;
wherein:
each AA¹, AA², and AA³ is independently an amino acid, or a derivative thereof,
SIL is a self-immolative linker,
is a bond to L (e.g., L¹, L⁴, L⁵, L⁶, or L⁷); and
** is a bond to PT.

In some embodiments, SIL is:

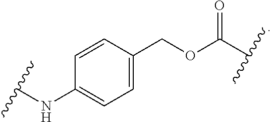 [SMILES: *Nc1ccc(COC(*)=O)cc1]

In some embodiments, EL is a dipeptide of the formula -AA¹-AA²-.

In some embodiments, provided herein is a compound of the formula:

IN-L⁵-AA¹-AA²-PT,
wherein each AA¹, and AA², is independently an amino acid, PT is a PTEFb inhibitor group, IN is an integrin binder, and L⁵ is a linker

In some embodiments, provided herein is a compound of the formula:

IN-L⁵-AA¹-AA²-SIL-PT,
wherein each AA¹ and AA², is independently an amino acid, SIL is a self-immolative linker, PT is a PTEFb inhibitor group, IN is an integrin binder, and L⁵ is a linker

In some embodiments, EL is a tripeptide of the formula -AA¹-AA²-AA³-.

In some embodiments, provided herein is a compound of the formula:

IN-L⁵-AA¹-AA²-AA³-PT,
wherein each AA¹, AA², and AA³ is independently an amino acid, PT is a PTEFb inhibitor group, IN is an integrin binder, and L⁵ is a linker

In some embodiments, provided herein is a compound of the formula:

IN-L⁵-AA¹-AA²-AA³-SIL-PT,
wherein each AA¹, AA², and AA³ is independently an amino acid, SIL is a self-immolative linker, PT is a PTEFb inhibitor group, IN is an integrin binder, and L⁵ is a linker

In some embodiments, the present invention provides compounds having a structure of formulae disclosed herein, or a pharmaceutically acceptable salt thereof, or a stereoisomer or mixture of stereoisomers thereof; wherein EL is cleaved by an enzyme from the class of proteases. In some embodiments, EL is cleaved by a serine protease or a cysteine protease. In some embodiments, EL is cleaved by a serine protease. In some embodiments, EL is cleaved by a cysteine protease. In some embodiments, EL is cleaved by a cathepsin (e.g., cathepsin B or legumain). In some embodiments, EL is cleaved by neutrophil elastase. In some embodiments, EL is cleaved by a tumor-associated enzyme (e.g., an enzyme that is overexpressed in a tumor microenvironment (e.g., by a tumor cell)). In some embodiments, EL is selectively cleaved in a tumor microenvironment. In some embodiments, EL is cleaved intracellularly.

In some embodiments, EL is cleaved by cathepsins, legumain, or neutrophil elastase.

In some embodiments, EL is cleaved by neutrophil elastase.

In some embodiments, EL has the formula: L-Asp-L-Pro-L-Val, L-Asn-L-Pro-L-Val, -Gly-L-Pro-L-Val-, L-Ala-L-Pro-L-Val-, L-Nva-L-Pro-L-Val-, L-His-L-Pro-L-Val-, L-Asp-L-Pro-L-Ile, L-Asn-L-Pro-L-Ile, -Gly-L-Pro-L-Ile-, L-Ala-L-Pro-L-Ile-, L-Nva-L-Pro-L-Ile-, L-His-L-Pro-L-Ile-, L-Asp-L-Pro-L-Leu, L-Asn-L-Pro-L-Leu, -Gly-L-Pro-L-Leu-, L-Ala-L-Pro-L-Leu-, L-Nva-L-Pro-L-Leu-, or L-His-L-Pro-L-Leu-. In some embodiments, EL has the formula L-Asp-L-Pro-L-Val, L-Asn-L-Pro-L-Val, -Gly-L-Pro-L-Val-, L-Ala-L-Pro-L-Val-, L-Nva-L-Pro-L-Val-, L-His-L-Pro-L-Val-, L-Asp-L-Pro-L-Ile, L-Asn-L-Pro-L-Ile, -Gly-L-Pro-L-Ile-, L-Ala-L-Pro-L-Ile-, L-Nva-L-Pro-L-Ile-, L-His-L-Pro-L-Ile-, L-Asp-L-Pro-L-Leu, L-Asn-L-Pro-L-Leu, -Gly-L-Pro-L-Leu-, L-Ala-L-Pro-L-Leu-, L-Nva-L-Pro-L-Leu-, or L-His-L-Pro-L-Leu- is cleavable by neutrophil elastase.

In some embodiments, EL has the formula L-Asp-L-Pro-L-Val, L-Asn-L-Pro-L-Val, -Gly-L-Pro-L-Val-, L-Ala-L-Pro-L-Val-, L-Nva-L-Pro-L-Val-, or L-His-L-Pro-L-Val-. Preference is given to conjugates wherein EL has the formula L-Asp-L-Pro-L-Val, L-Asn-L-Pro-L-Val, or -Gly-L-Pro-L-Val-.

In some embodiments, EL is of Formula (EL-1a):

L-Asp-L-Pro-L-Val Formula (EL-1a).

In some embodiments, EL is of Formula (EL-1b):

L-Asn-L-Pro-L-Val Formula (EL-1b).

Also provided herein are conjugates wherein EL is of the formula L-Asp*-L-Pro-L-Val, wherein the L-Asp* residue is a masked aspartate residue (e.g., is prodrug that is cleaved enzymatically or chemically). In some embodiments, an L-Asp* prodrug contains an inert masking moiety (e.g., an alkylamine or alkylaminium ester). In some embodiments, an L-Asp* prodrug contains a cleavable linkage (e.g., an ester linkage) to an integrin binder. In some embodiments, the alkyl ester prodrug is cleaved in vivo to L-Asp. All metabolites of prodrugs disclosed herein are also considered within the scope of the invention, as are their uses in treating diseases or disorders.

In some embodiments, an L-Asp* prodrug (e.g., wherein EL is L-Asp*-L-Pro-L-Val) is an alkyl ester, wherein the alkyl ester is optionally substituted with -L⁶IN or -NHL⁶IN, wherein L⁶ is a spacer (e.g., a simple spacer, a polymeric spacer, or elongated spacer as described herein) and IN is an integrin binder. In some embodiments, the alkyl ester is substituted with -L⁶IN or -NHL⁶-IN wherein L⁶ is a substituted or unsubstituted C₁₋₃₀ alkyl or substituted or unsubstituted heteroalkyl and IN is an integrin binder.

In some embodiments, EL is of Formula (EL-1c):

Gly-L-Pro-L-Val Formula (EL-1c).

In some embodiments, EL is cleaved by cathepsin.

In some embodiments, EL has the formula: -L-Val-L-Cit-, L-Phe-L-Cit-, -L-Leu-L-Cit-, -L-Val-L-Ala-, -L-Phe-L-Lys-, -L-Ala-L-Lys-, or -L-Val-L-Lys-. In some embodiments, EL has the formula L-Val-L-Cit-, L-Phe-L-Cit-, -L-Leu-L-Cit-, -L-Val-L-Ala-, -L-Phe-L-Lys-, -L-Ala-L-Lys-, or -L-Val-L-Lys is cleavable by cathepsin. In some embodiments, EL has the formula L-Val-L-Cit.

In some embodiments, EL is of Formula (EL-2a):

L-Val-L-Cit Formula (EL-2a).

In some embodiments, EL is of Formula (EL-2a):

L-Val-L-Ala Formula (EL-2a).

In some embodiments, EL is cleaved by legumain.

In some embodiments, EL has the formula: -L-Ala-L-Ala-L-Asp or -L-Ala-L-Ala-L-Asn, -L-Ala-L-Asp-L-Asn, L-Ala-L-Asn, or -L-Asp-L-Asn, wherein each amino acid is, independently of one another, optionally, N-alkylated with C₁₋₃ alkyl. In some embodiments, EL has the formula -L-Ala-L-Ala-L-Asp or -L-Ala-L-Ala-L-Asn, -L-Ala-L-Asp-L-Asn, L-Ala-L-Asn, or -L-Asp-L-Asn, wherein each amino acid is, independently of one another, optionally, N-alkylated with C₁₋₃ alkyl is cleavable by legumain. In some embodiments, EL has the formula: -L-Ala-L-Ala-L-Asp or -L-Ala-L-Ala-L-Asn. In some embodiments, EL has the formula -L-Ala-L-Ala-L-Asp. In some embodiments, EL has the formula -L-Ala-L-Ala-L-Asn.

In some embodiments, EL has the formula: L-Ala-L-N-Me-Ala-L-Asn, L-Ala-D-His-L-Asn, L-Ala-D-Asp-L-Asn, L-Ala-D-Ala-L-Asn, or L-Ala-D-Ser-L-Asn. In some embodiments, EL having the formula L-Ala-L-N-Me-Ala-L-Asn, L-Ala-D-His-L-Asn, L-Ala-D-Asp-L-Asn, L-Ala-D-Ala-L-Asn, or L-Ala-D-Ser-L-Asn is cleavable by legumain. In some embodiments, EL has the formula L-Ala-L-N-Me-Ala-L-Asn. In some embodiments, EL has the formula L-Ala-D-N-Me-Ala-L-Asn.

In some embodiments, EL is of Formula (EL-3a):

L-Ala-L-N-Me-Ala-L-Asn Formula (EL-3a).

### Integrin Binders (IN)

In some embodiments, the present invention provides compounds having a structure of formulae disclosed herein, or a pharmaceutically acceptable salt thereof, or a stereoisomer or mixture of stereoisomers thereof, wherein IN is an integrin binder comprising peptides, proteins, antibodies, or small molecules. In some embodiments, the molecular weight of such peptides, proteins, antimobodies, or small molecules is <1 kD.

In some embodiments, the present invention provides compounds having a structure of formulae disclosed herein, or a pharmaceutically acceptable salt thereof, or a stereoisomer or mixture of stereoisomers thereof, wherein IN is a peptidic or peptidomimetic integrin binder.

In some embodiments, the present invention provides compounds having a structure of formulae disclosed herein, or a pharmaceutically acceptable salt thereof, or a stereoisomer or mixture of stereoisomers thereof, wherein IN is an integrin binder comprising non-peptides, non-antibodies, or small molecules. In some embodiments, molecular weight of such non-peptides, non-proteins, non-antimobodies, or small molecules (e.g., <1 kD). In some embodiments, IN is a small molecule integrin binder. In some embodiments, IN is a small molecule integrin binder having two or more urea groups (—NHC(O)NH—). In some embodiments, IN is a small molecule αᵥβ₃ integrin binder.

In some embodiments, IN is a linear integrin binding peptide. In some embodiments, IN is a macrocyclic integrin binding peptide. In some embodiments, the linear integrin binding peptide is a constrained macrocyclic integrin binding peptide. In some embodiments, IN is a non-peptidic or non-peptidomimetic integrin binder. In some embodiments, IN is a tumor-binding moiety. In some embodiments, IN binds to both tumor and non-tumor cells. In some embodiments, IN binds rapidly dividing cells (e.g., tumor cells or other hyperproliferating cells). In some embodiments, IN is an αᵥβ₃-binding moiety. In some embodiments, IN is IN-1a or IN-1b:

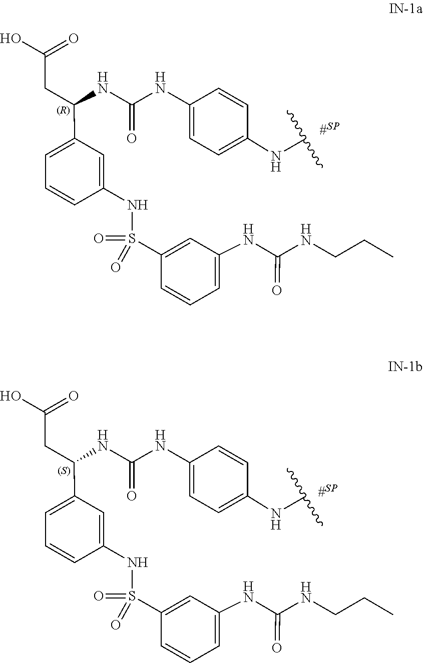

or a pharmaceutically acceptable salt thereof; or a stereoisomer or mixture of stereoisomers thereof; wherein #SP denotes a bond to a spacer, e.g., one of L², L³, L⁴, L⁵, L⁶, or L⁷.

In some embodiments, the (R)-isomer (e.g., IN-1a) is a strongly-binding epimer of an integrin binder, and the (S)-isomer (e.g., IN-1b) is a weakly-binding epimer of an integrin binder. In some embodiments, IN is the strongly binding epimer (e.g., IN-1a). In some embodiments, IN is the weakly-binding epimer (e.g., IN-1b).

In some embodiments, IN has a structure:

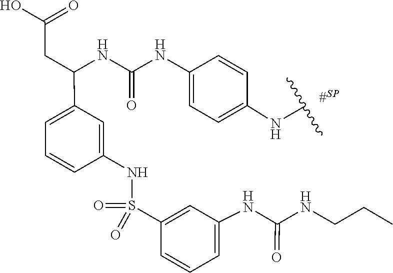 [SMILES: *Nc1ccc(NC(=O)NC(CC(=O)O)c2cccc(NS(=O)(=O)c3cccc(NC(=O)NCCC)c3)c2)cc1]

or a pharmaceutically acceptable salt thereof; or a stereoisomer or mixture of stereoisomers thereof; wherein #^{SP} denotes a bond to a spacer, e.g., one of L², L³, L⁴, L⁵, L⁶, or L⁷.

### PTEFb Inhibitor (PT)

In some embodiments, PT is a radical of a PTEFb inhibitor. In some embodiments, PT is a radical of a macrocyclic or polycyclic small molecule PTEFb inhibitor. In some embodiments, PT is a radical of a polycyclic small molecule PTEFb inhibitor. In some embodiments, PT is a radical of a macrocyclic small molecule PTEFb inhibitor.

In some embodiments, PT is a radical of a sulfoximine-linked PTEFb inhibitor. In some embodiments, PT is a radical of a sulfoximine-linked macrocyclic or polycyclic small molecule PTEFb inhibitor. In some embodiments, PT is a radical of a sulfoximine-linked polycyclic small molecule PTEFb inhibitor. In some embodiments, PT is a radical of a sulfoximine-linked macrocyclic small molecule PTEFb inhibitor.

In some embodiments, PT is a radical of a PTEFb inhibitor having a structure represented by the formula:

—(SFX)-(L^{A})-(Ring A)-(L^{B})-(Ring B)-(L^{C})-(Ring C)-(L^{D})-; (iv)
wherein:
SFX is a sulfoximine moiety (e.g., —N═S(═O)(C₁₋₆alkyl)-)
L^{A} is a bond or C₁₋₆ alkyl;
Ring A is an optionally substituted carbocycle or optionally substituted heterocycle (e.g., optionally substituted phenyl or optionally substituted heteroaryl);
L^{B} is a bond, optionally substituted C₁₋₆alkyl, or optionally substituted heteroalkyl (e.g., —CH₂—, —C(═O)NH— —NH—, —N(CH₃)—, —NHC(═O)—, —O—, or —S—;);
Ring B is an optionally substituted carbocycle or optionally substituted heterocycle (e.g., optionally substituted phenyl or optionally substituted heteroaryl);
L^{C} is a bond, optionally substituted C₁₋₆alkyl, or optionally substituted heteroalkyl (e.g., —CH₂—, —C(═O)NH— —NH—, —N(CH₃)—, —NHC(═O)—, —O—, or —S—;);
Ring C is an optionally substituted carbocycle or optionally substituted heterocycle (e.g., optionally substituted phenyl or optionally substituted heteroaryl); and
L^{D} is absent; or an optionally substituted heteroalkyl bonded to Ring A, thereby forming an optionally substituted macrocyclic Ring D (e.g., a ring comprising 12 to 20 atoms selected from C, N, O, and S).

In some embodiments, PT has a structure represented by the formula:

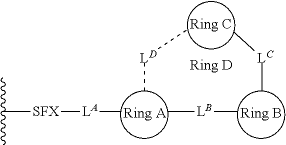

wherein if L^{D} is present, it connects Ring C and Ring A to form a macrocyclic Ring D.

In some embodiments, L^{D} is absent. In some embodiments, L^{D} is an optionally substituted heteroalkyl group. In some embodiments, L^{D} is a heteroalkyl group comprising 2 to 12 atoms selected from C, N, O, and S. In some embodiments, L^{D} is a heteroalkyl group of the formula —O—(C₁₋₆alkyl)-O—, —NH—(C₁₋₆alkyl)-O—, or —NH—(C₁₋₆alkyl)-NH—. In some embodiments, L^{D} is a heteroalkyl group of the formula —O—(C₁₋₆alkyl)-O—. In some embodiments, L^{C} is a bond. In some embodiments, L^{B} is —CH₂—, —C(═O)NH— —NH—, —N(CH₃)—, —NHC(═O)—, —O—, or —S—. In some embodiments, L^{B} is —CH₂—, —NH—, —N(CH₃)—, —O—, or —S—. In some embodiments, L^{B} is —CH₂—, —NH—, —O—, or —S—. In some embodiments, L^{B} is —CH₂—. In some embodiments, L^{B} is —NH—. In some embodiments, L^{B} is —O—. In some embodiments, L^{A} is C₁₋₆ alkyl. In some embodiments, L^{A} is —CH₂.

In some embodiments, PT is a radical of a PTEFb inhibitor having a structure represented by the formula:

—(SFX)-(L^{A})-(Ring A)-(L^{B})-(Ring B)-(L^{C})-(Ring C)-(L^{D})-; (iv)
wherein:
SFX is a sulfoximine moiety (e.g., —N═S(═O)(C₁₋₆alkyl)-)
L^{A} is —CH₂—;
L^{B} —CH₂—, —C(═O)NH— —NH—, —N(CH₃)—, —NHC(═O)—, —O—, or —S—;
L^{C} is a bond; and
L^{D} is absent or —O—(C₁₋₆alkyl)-O—.

In some embodiments, Ring A is a carbocycle or heterocycle. In some embodiments, Ring A is a six-membered carbocycle or heterocycle. In some embodiments, Ring A is an optionally substituted phenyl or optionally substituted six-membered heteroaryl (e.g., pyridine, pyrazine, pyridazine, pyrimidine, triazine, or tetrazine). In some embodiments, Ring A is an optionally substituted phenyl or an optionally substituted pyridine.

In some embodiments, Ring B is a carbocycle or heterocycle. In some embodiments, Ring B is a six-membered carbocycle or heterocycle. In some embodiments, Ring B is an optionally substituted phenyl or optionally substituted six-membered heteroaryl (e.g., pyridine, pyrazine, pyridazine, pyrimidine, triazine, or tetrazine). In some embodiments, Ring B is an optionally substituted phenyl or an optionally substituted pyridine. In some embodiments, Ring B is an optionally substituted six-membered heteroaryl. In some embodiments, Ring B is an optionally substituted pyridine, pyrimidine, or triazine.

In some embodiments, Ring C is a carbocycle or heterocycle. In some embodiments, Ring C is a six-membered carbocycle or heterocycle. In some embodiments, Ring C is an optionally substituted phenyl or optionally substituted six-membered heteroaryl (e.g., pyridine, pyrazine, pyridazine, pyrimidine, triazine, or tetrazine). In some embodiments, Ring C is an optionally substituted phenyl or an optionally substituted pyridine.

In some embodiments, PT is a radical of a PTEFb inhibitor having a structure represented by the formula:

—(SFX)-(L^{A})-(Ring A)-(L^{B})-(Ring B)-(L^{C})-(Ring C)-(L^{D})-; (iv)
wherein:
SFX is a sulfoximine moiety (e.g., —N═S(═O)(C₁₋₆alkyl)-)
L^{A} is —CH₂—;
Ring A is optionally substituted phenyl or optionally substituted pyridine;
L^{B} —NH—
Ring B is an optionally substituted pyridine, optionally substituted pyrimidine, or optionally substituted triazine;
L^{C} is a bond;
Ring C is an optionally substituted phenyl; and
L^{D} is absent or —O—(C₁₋₆alkyl)-O—.

In some embodiments, PT is a CDK9 inhibitor. In some embodiments, PT comprises a sulfoximine moiety. In some embodiments, the sulfoximine moiety of PT is enzymatically cleavable. In some embodiments, the sulfoximine moiety of PT is cleaved (e.g., selectively) by tumor stromal enzymes (e.g., neutrophil elastase).

In some embodiments, the present invention provides compounds having a structure of formulae disclosed herein, wherein PT has a structure of the formula:

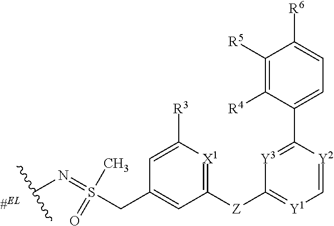

or a pharmaceutically acceptable salt thereof; or a stereoisomer or mixture of stereoisomers thereof;
wherein:
   #^{EL} denotes a bond to EL (or SIL);
   X¹ is CH, CF, or N;
   Y¹ is CH, CF, or N;
   Y² is CH, CF, or N;
   Y³ is CH, CF, or N;
   Z is —CH₂—, —C(═O)NH— —NH—, —N(CH₃)—, —NHC(═O)—, —O—, or —S—;
   R³ is hydrogen, halogen, —OH, or —O—C₁₋₄alkyl;
   R⁴ is hydrogen, halogen, —OH, or —O—C₁₋₄alkyl;
   or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
   R⁵ is hydrogen, halogen, —OH, or —O—C₁₋₄alkyl;
   or R³ and R⁵ are taken together to form a bivalent radical of the formula —O—C₂₋₁₀ alkyl-O—, —NH—C₂₋₁₀ alkyl-O—, or —NH—C₂₋₁₀ alkyl-NH—; and
   R⁶ is hydrogen, halogen, —OH, or —O—C₁₋₄alkyl.

In some embodiments, wherein PT is:

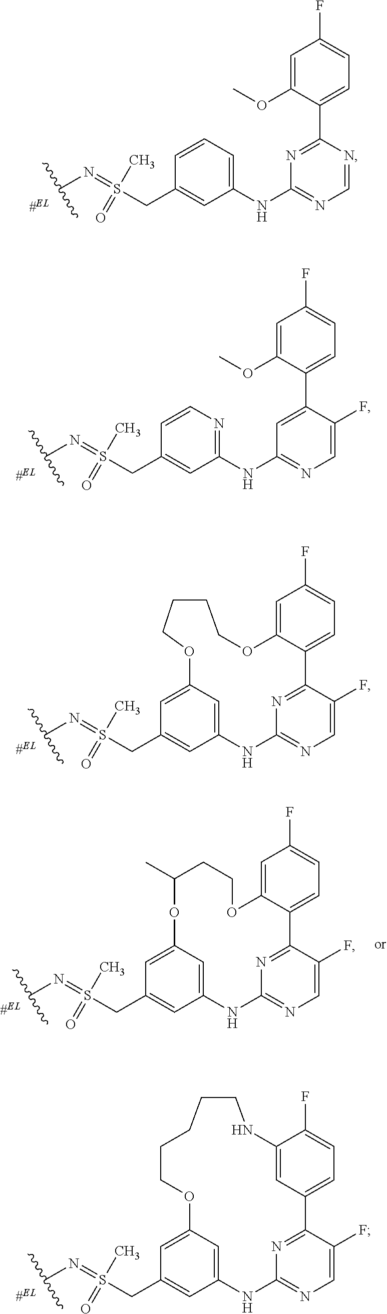 [SMILES: *N=S(C)(=O)Cc1cccc(Nc2ncnc(-c3ccc(F)cc3OC)n2)c1 ; *N=S(C)(=O)Cc1ccnc(Nc2cc(-c3ccc(F)cc3OC)c(F)cn2)c1 ; *N=S(C)(=O)Cc1cc2cc(c1)OCCCCOc1cc(F)ccc1-c1nc(ncc1F)N2 ; *N=S(C)(=O)Cc1cc2cc(c1)OC(C)CCOc1cc(F)ccc1-c1nc(ncc1F)N2 ; "or" ; *N=S(C)(=O)Cc1cc2cc(c1)OCCCCCNc1cc(ccc1F)-c1nc(ncc1F)N2]

wherein #^{EL} denotes a bond to EL or SIL.

In some embodiments, PT is:

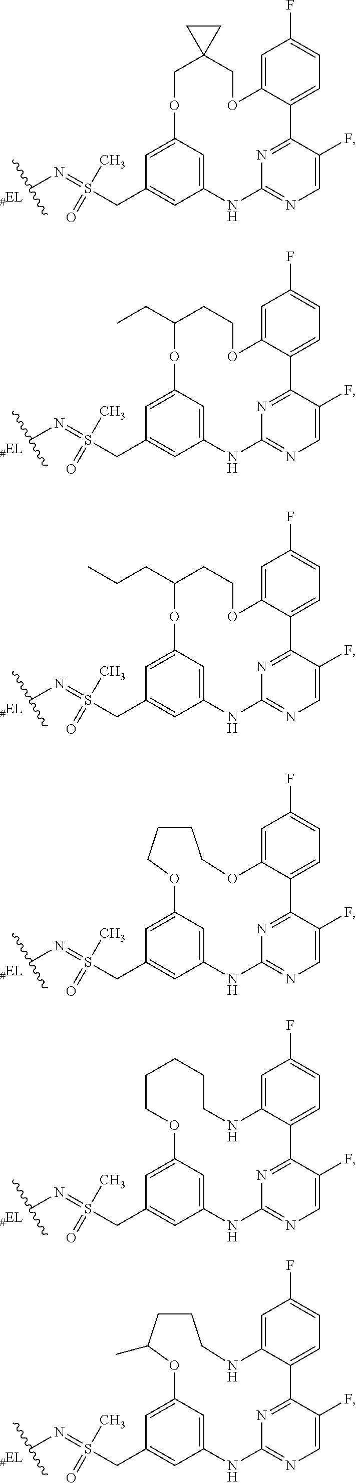

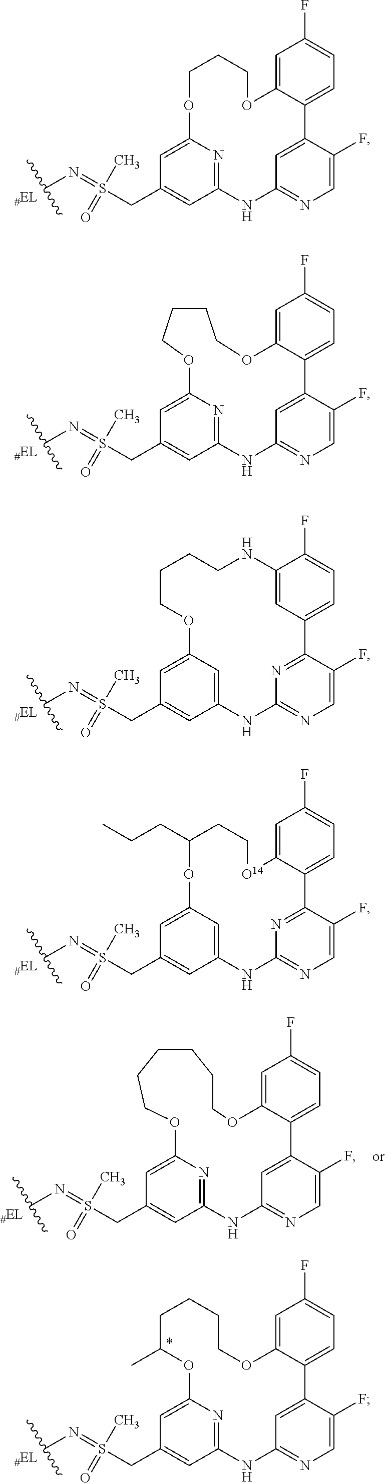

wherein #^{EL} denotes a bond to EL or SIL.

In some embodiments, PT is:

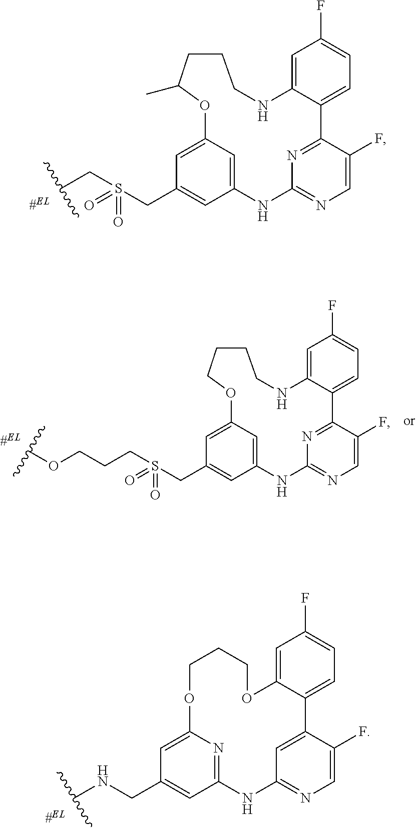 [SMILES: *CS(=O)(=O)Cc1cc2cc(c1)OC(C)CCCNc1cc(F)ccc1-c1nc(ncc1F)N2 ; *OCCCS(=O)(=O)Cc1cc2cc(c1)OCCCCNc1cc(F)ccc1-c1nc(ncc1F)N2 ; "or" ; *NCc1cc2nc(c1)OCCCOc1cc(F)ccc1-c1cc(ncc1F)N2]

In some embodiments, the present invention provides compounds having a structure of formulae disclosed herein, wherein PT is:

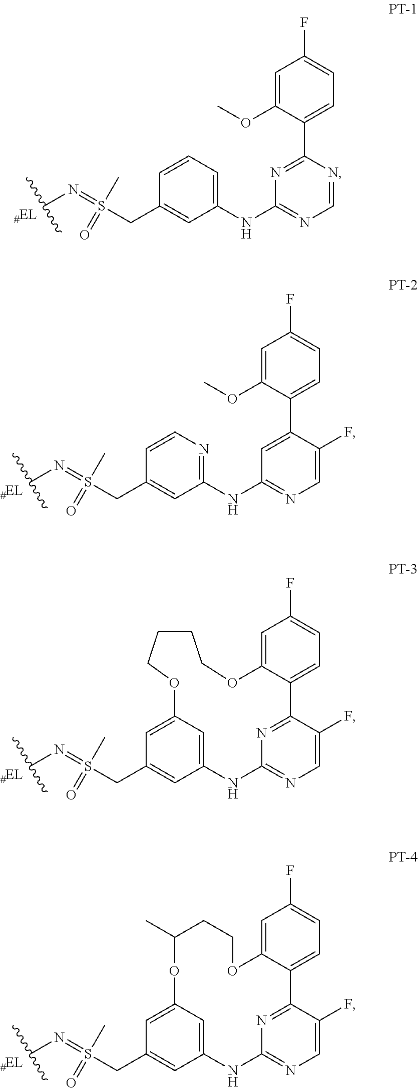 [SMILES: *N=S(C)(=O)Cc1cccc(Nc2ncnc(-c3ccc(F)cc3OC)n2)c1 ; *N=S(C)(=O)Cc1ccnc(Nc2cc(-c3ccc(F)cc3OC)c(F)cn2)c1 ; *N=S(C)(=O)Cc1cc2cc(c1)OCCCCOc1cc(F)ccc1-c1nc(ncc1F)N2 ; *N=S(C)(=O)Cc1cc2cc(c1)OC(C)CCOc1cc(F)ccc1-c1nc(ncc1F)N2]

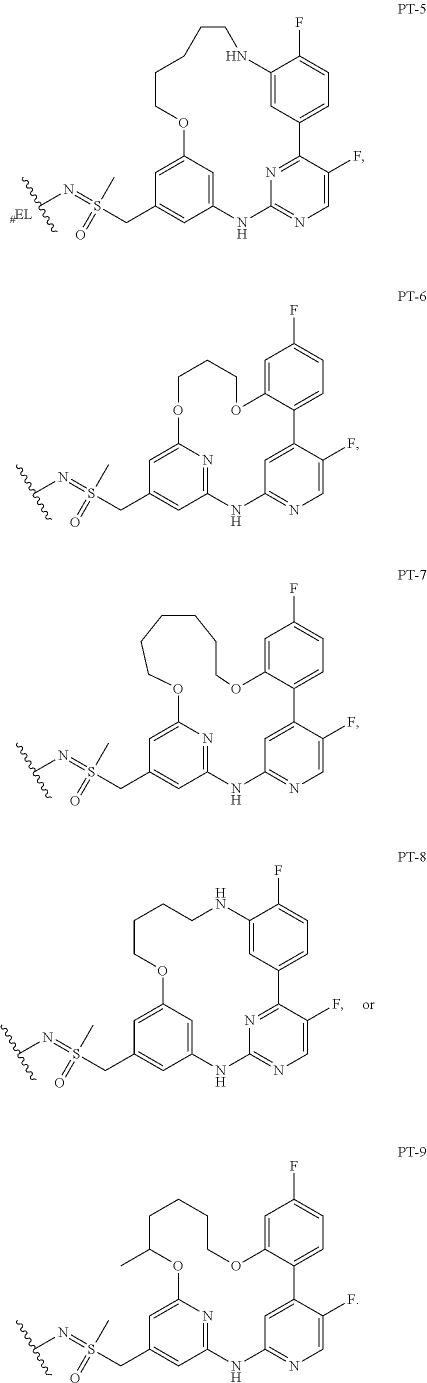 [SMILES: *N=S(C)(=O)Cc1cc2cc(c1)OCCCCCNc1cc(ccc1F)-c1nc(ncc1F)N2 ; *N=S(C)(=O)Cc1cc2nc(c1)OCCCOc1cc(F)ccc1-c1cc(ncc1F)N2 ; *N=S(C)(=O)Cc1cc2nc(c1)OCCCCCCOc1cc(F)ccc1-c1cc(ncc1F)N2 ; *N=S(C)(=O)Cc1cc2cc(c1)OCCCCNc1cc(ccc1F)-c1nc(ncc1F)N2 ; *N=S(C)(=O)Cc1cc2nc(c1)OC(C)CCCCOc1cc(F)ccc1-c1cc(ncc1F)N2]

In some embodiments, the present invention provides compounds having a structure of formulae disclosed herein, wherein PT is:

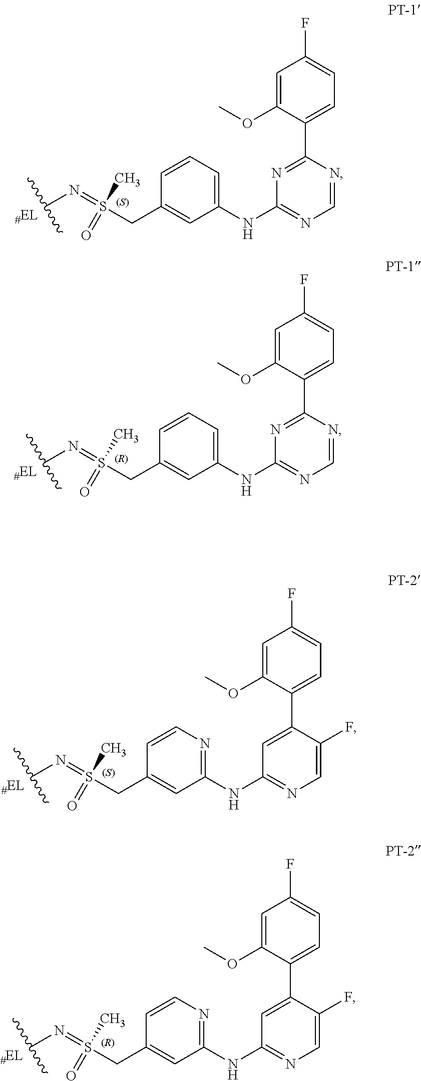

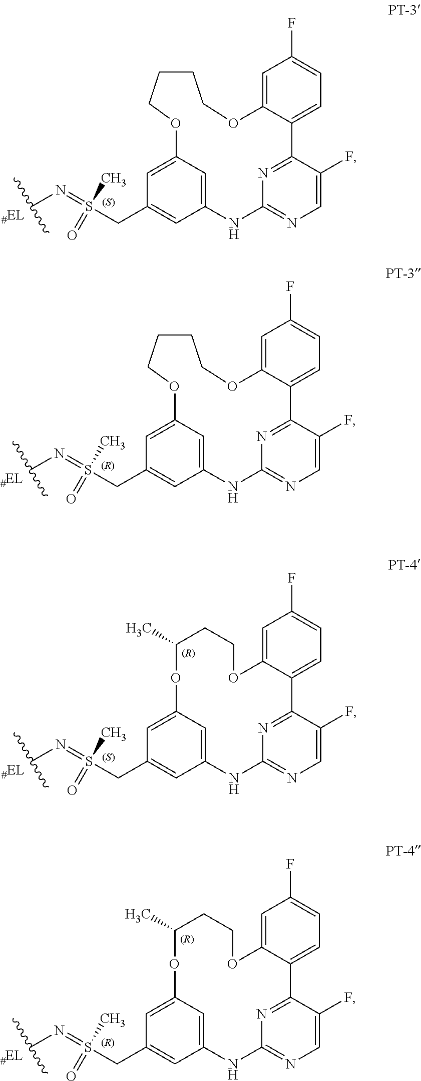

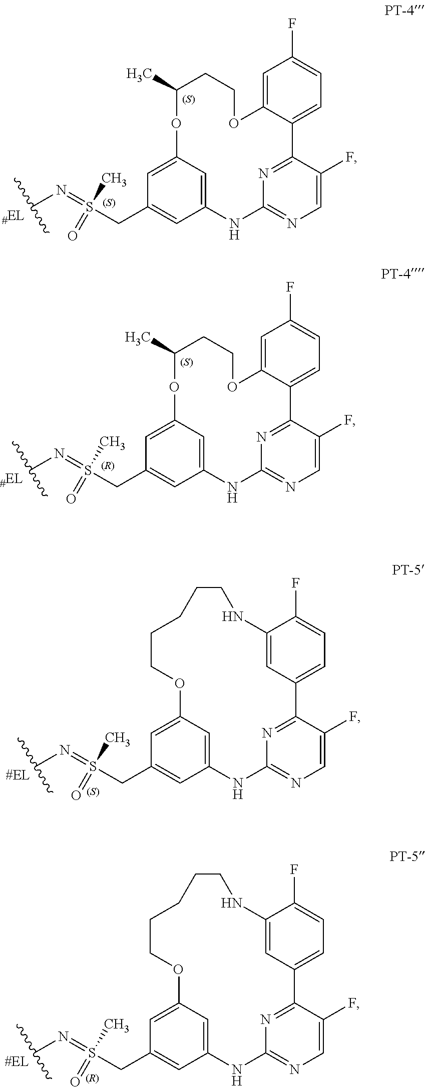

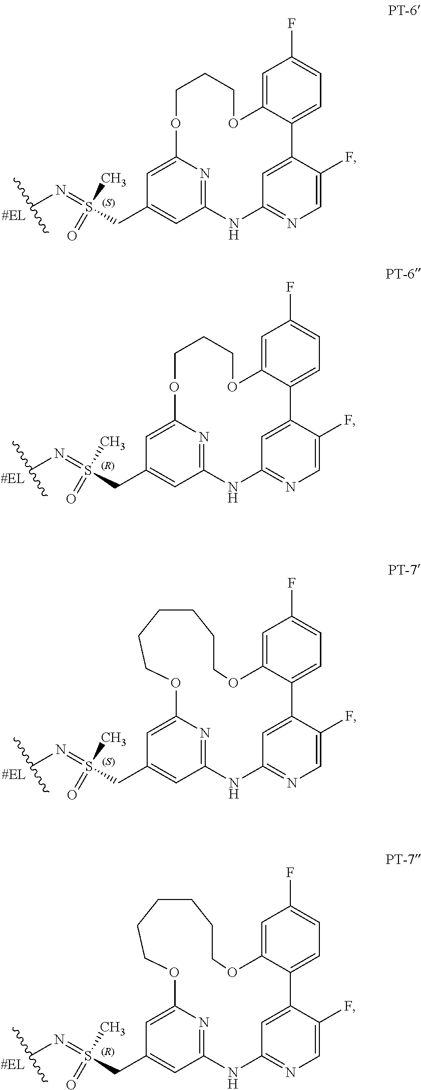

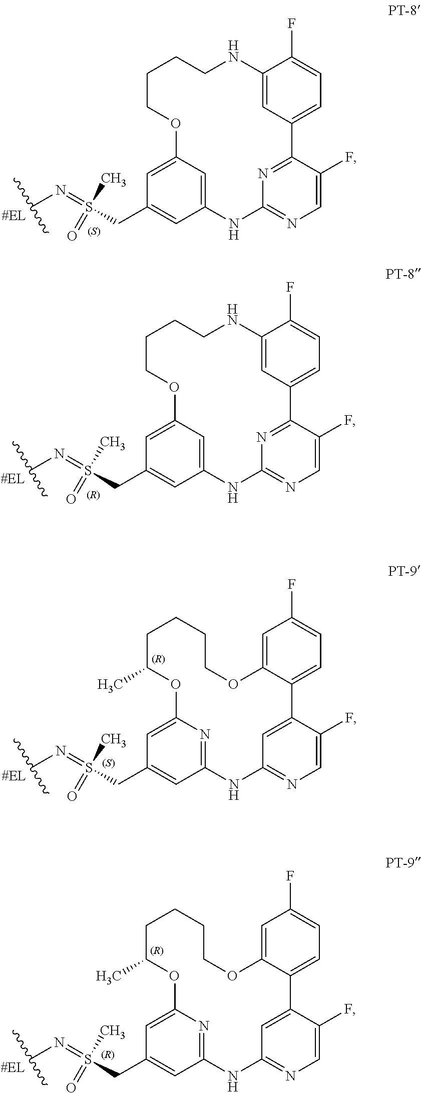

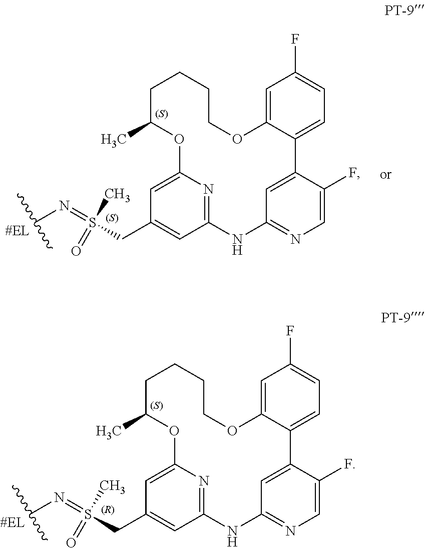

Any combination of the groups described above for the various variables is contemplated herein. Throughout the specification, groups and substituents thereof are chosen by one skilled in the field to provide stable moieties and compounds.

In some embodiments, provided herein is a compound having a structure:

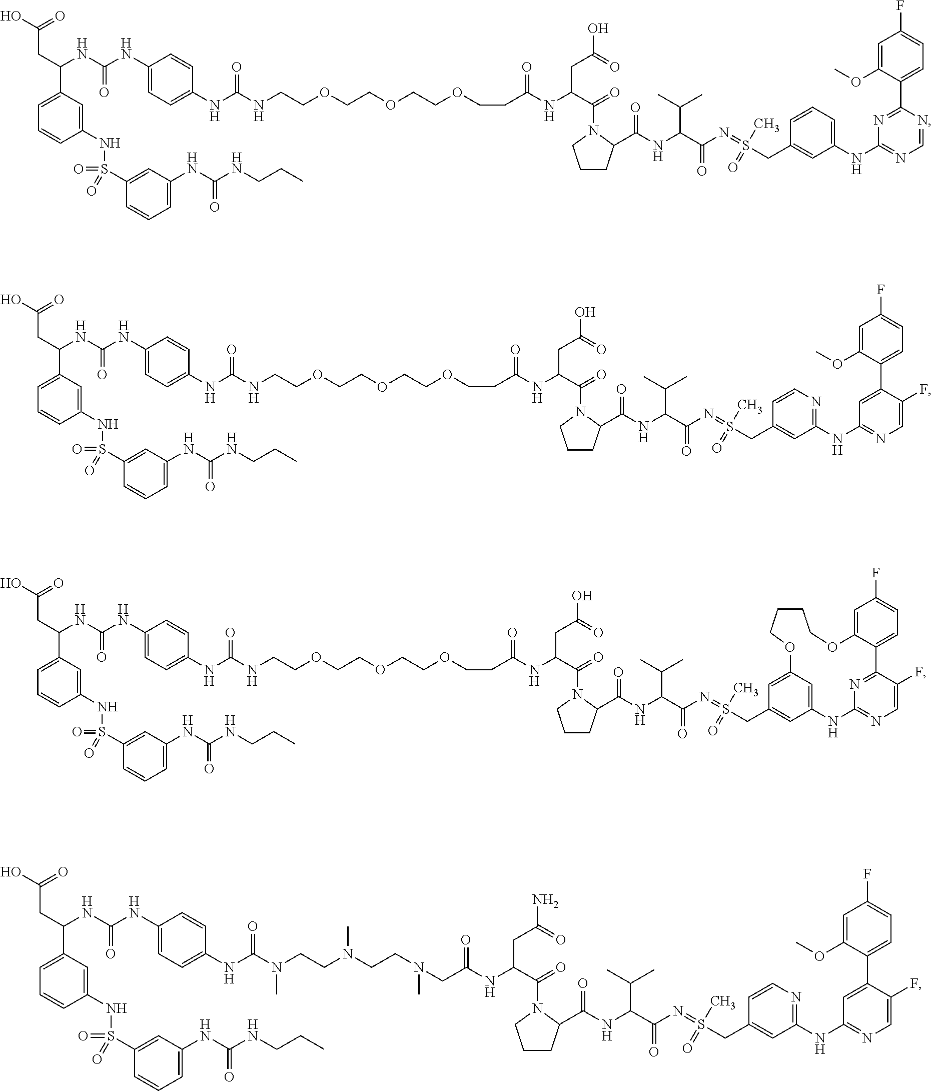

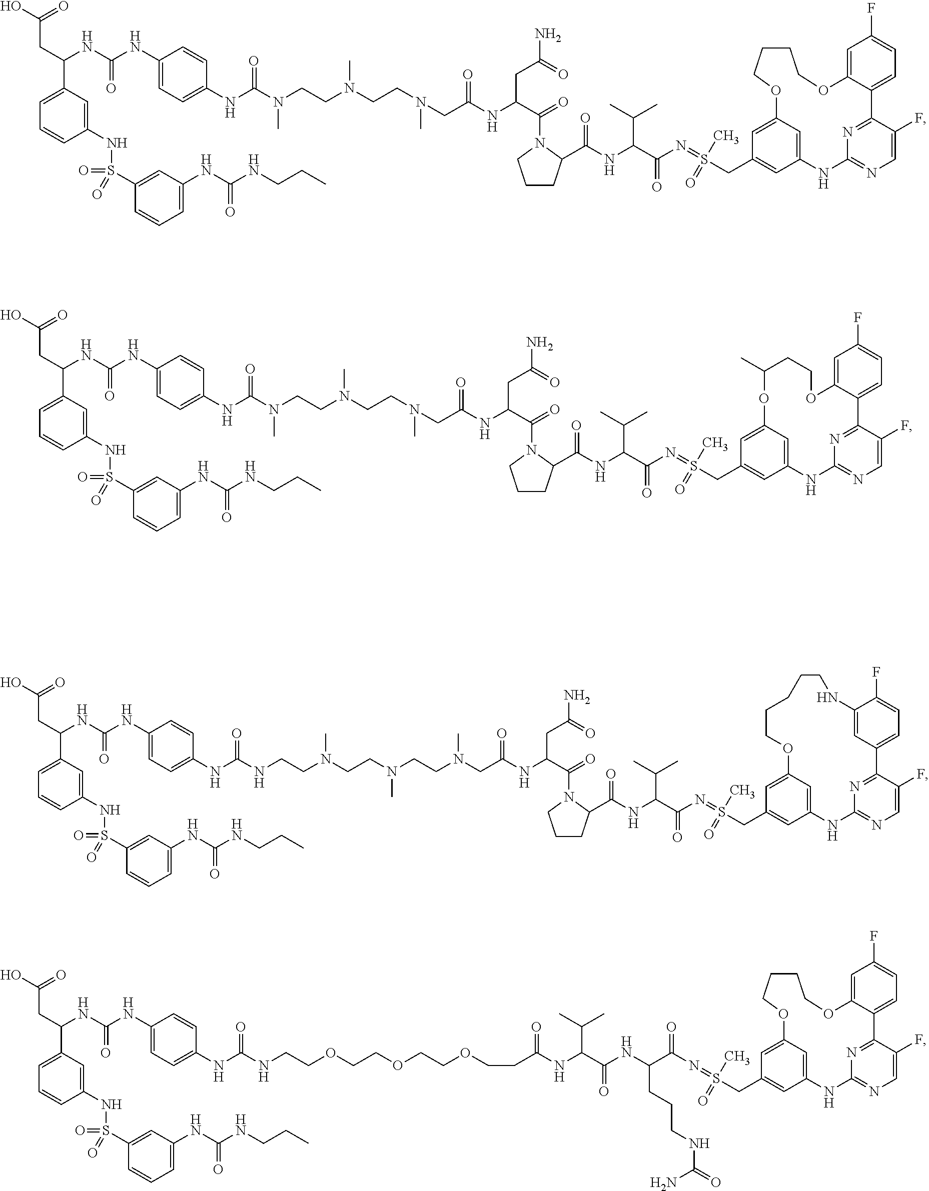

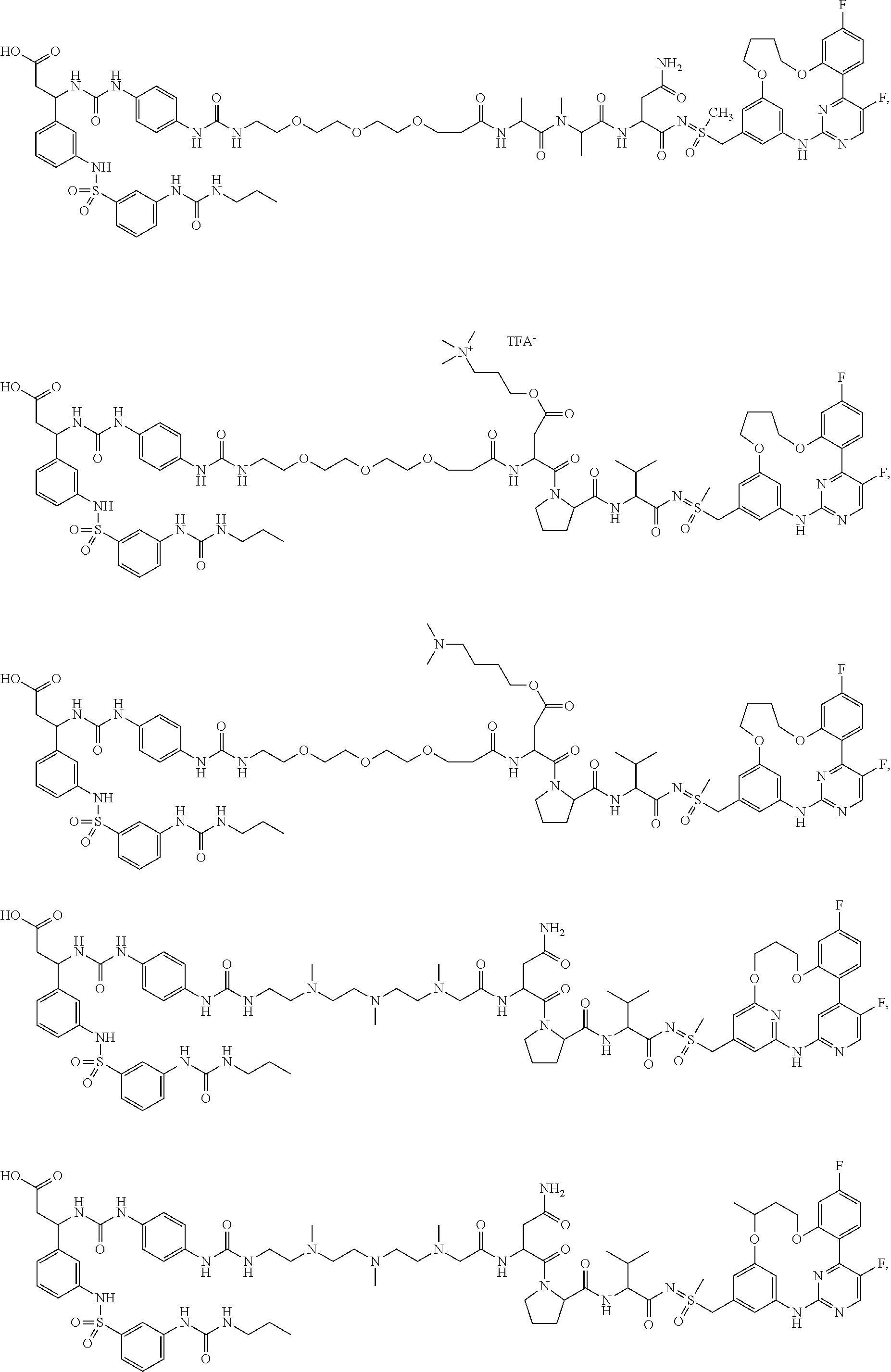

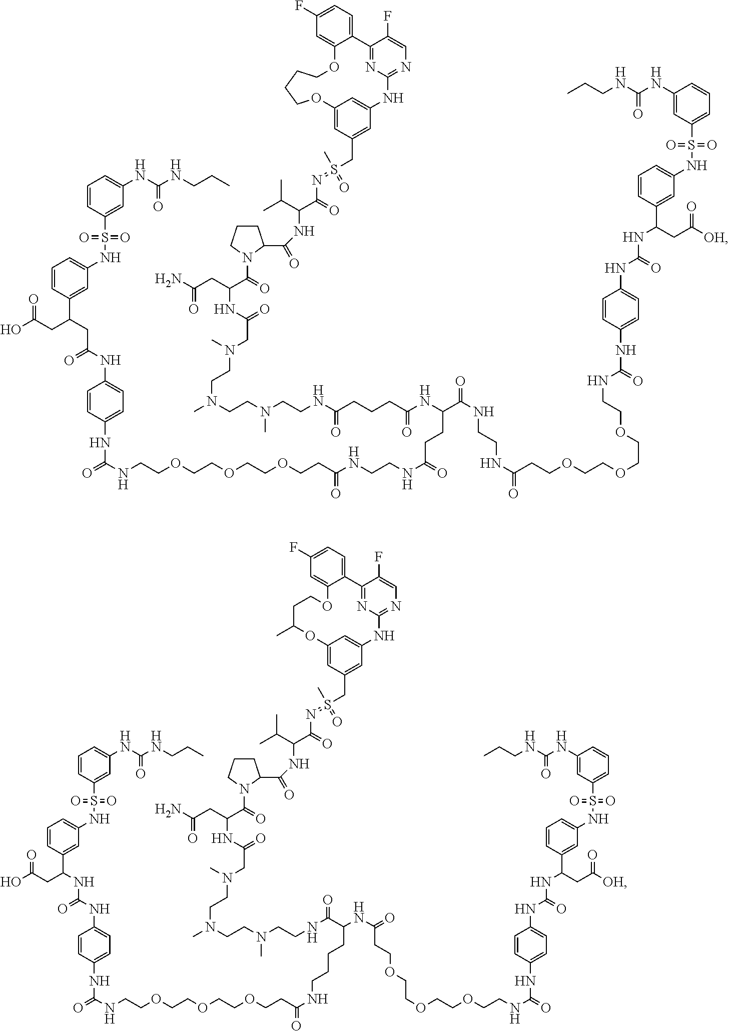

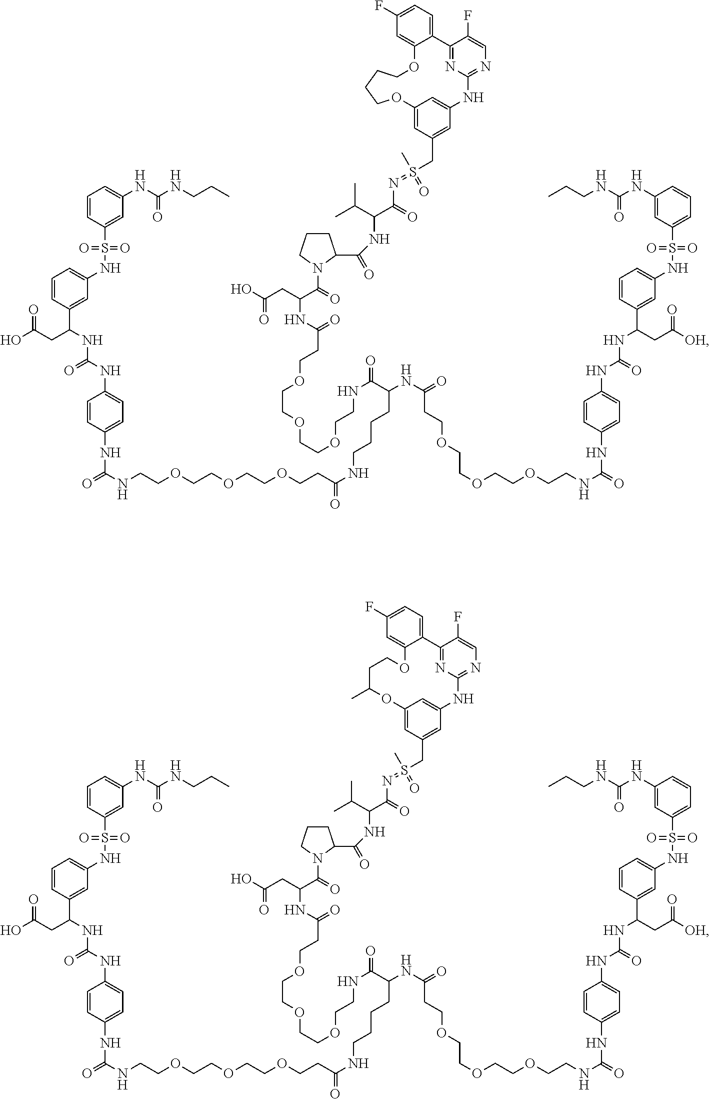

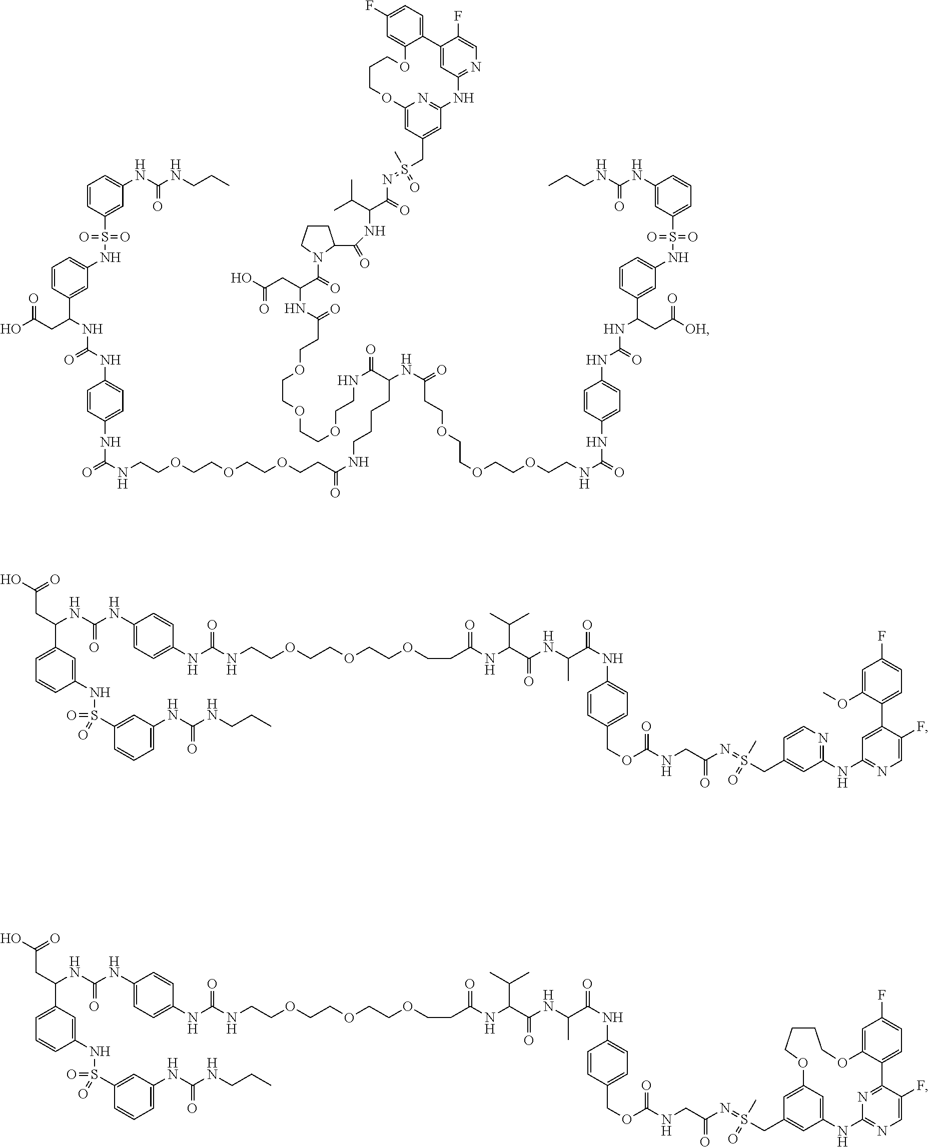

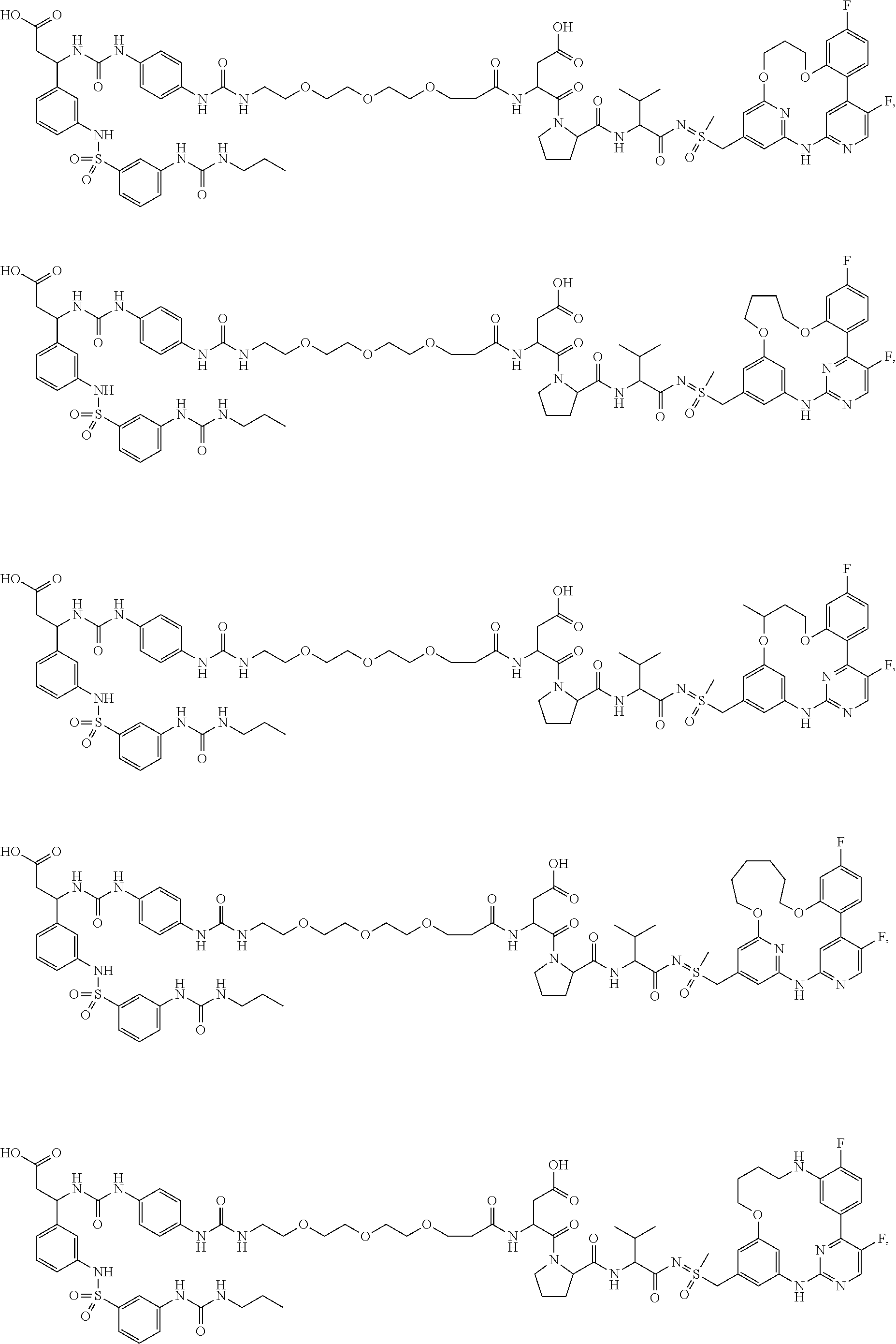

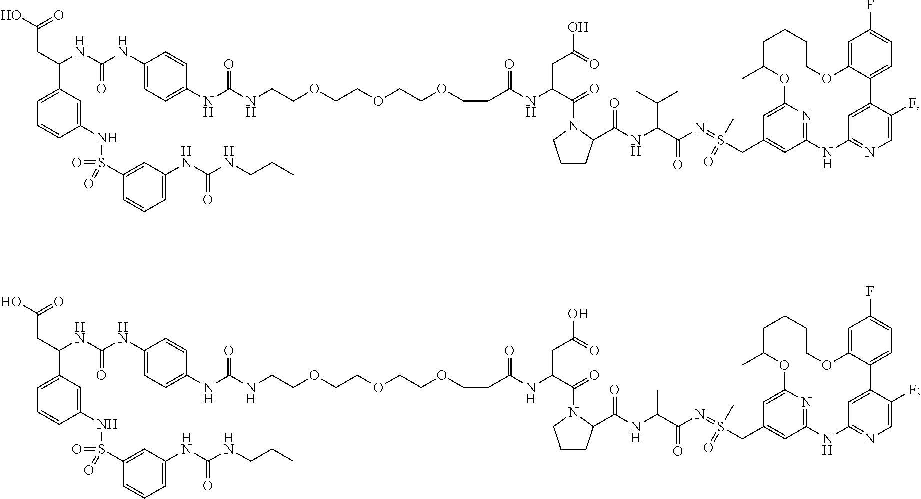 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C(=O)N=S(C)(=O)Cc4cc5nc(c4)OC(C)CCCCOc4cc(F)ccc4-c4cc(ncc4F)N5)C(C)C)cc3)c2)c1 ; CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C)C(=O)N=S(C)(=O)Cc4cc5nc(c4)OC(C)CCCCOc4cc(F)ccc4-c4cc(ncc4F)N5)cc3)c2)c1]

or a stereoisomer thereof; or a mixture of stereoisomers thereof; or an isotopic variant thereof; or
a pharmaceutically acceptable salt or solvate thereof.

### Pharmaceutically Acceptable Salts

In some embodiments, provided herein is a pharmaceutically acceptable salt of a compound whose structure is disclosed herein.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In some embodiments, pharmaceutically acceptable salts are obtained by reacting a conjugate described herein with an acid. In some embodiments, the conjugate described herein (e.g., free base form) is basic and is reacted with an organic acid or an inorganic acid. Inorganic acids include, but not limited to, hydrochloric acid, hydrobromic acid, sulfuric acid, phosphoric acid, nitric acid, and metaphosphoric acid. Organic acids include, but not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (-L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (-L); salicylic acid; sebacic acid; stearic acid; succinic acid; sulfuric acid; tartaric acid (+L); thiocyanic acid; toluenesulfonic acid (p); and undecylenic acid.

In some embodiments, pharmaceutically acceptable salts of the inventive compounds especially include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, formic acid, acetic acid, trifluoroacetic acid, propionic acid, succinic acid, fumaric acid, maleic acid, lactic acid, tartaric acid, malic acid, citric acid, gluconic acid, benzoic acid, and embonic acid.

In some embodiments, a conjugate described herein is prepared as a chloride salt, sulfate salt, bromide salt, mesylate salt, maleate salt, citrate salt or phosphate salt.

In some embodiments, pharmaceutically acceptable salts are obtained by reacting a conjugate described herein with a base. In some embodiments, the conjugate described herein is acidic and is reacted with a base. In such situations, an acidic proton of the conjugate described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt. In some embodiments, the compounds provided herein are prepared as a sodium salt, e.g., a monosodium salt, a disodium salt, a trisodium salt, or a tetrasodium salt. In some embodiments, the salt is a disodium or trisodium salt. In some embodiments, the salt is a trifluoroacetate (TFA) salt, e.g., a mono-TFA, di-TFA, tri-TFA, or tetra-TFA salt.

In some embodiments, pharmaceutically acceptable salts of the inventive compounds also include salts derived from conventional bases, by way of example and with preference alkali metal salts (e.g. sodium and potassium salts), alkaline earth metal salts (e.g. calcium and magnesium salts), zinc salts and ammonium salts derived from ammonia or organic amines having 1 to 20 carbon atoms, by way of example and with preference ethylamine, diethylamine, triethylamine, N,N-ethyldiisopropylamine, monoethanolamine, diethanolamine, triethanolamine, dimethylaminoethanol, diethylaminoethanol, tris(hydroxymethyl)aminomethane, choline, benzalkonium, procaine, dibenzylamine, dicyclohexylamine, N-methylmorpholine, N-methylpiperidine, arginine, lysine, and 1,2-ethylenediamine.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, the context of the invention are described as those forms of the inventive compounds which form a complex in the solid or liquid state by coordination with solvent molecules. Hydrates are a specific form of the solvates in which the coordination is with water. Solvates preferred in the context of the present invention are hydrates.

The methods and formulations described herein include the use of N-oxides (if appropriate), or pharmaceutically acceptable salts of compounds having the structure of Formula (I), as well as active metabolites of these compounds having the same type of activity.

In some embodiments, sites on the organic radicals (e.g. alkyl groups, aromatic rings) of compounds of formulae disclosed herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the present invention also encompasses all suitable isotopic variants of the inventive compounds. An isotopic variant of an inventive compound is understood here to mean a compound in which at least one atom within the inventive compound has been exchanged for another atom of the same atomic number, but with a different atomic mass than the atomic mass which usually or predominantly occurs in nature. Examples of isotopes which can be incorporated into an inventive compound are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, particularly compounds labelled with ³H, ¹⁴C, or ¹⁸F isotopes are suitable for the purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the inventive compounds may therefore possibly also constitute a preferred embodiment of the present invention. Isotopic variants of the inventive compounds can be prepared by commonly used processes known to those skilled in the art, for example by the methods described further down and the procedures described in the working examples, by using corresponding isotopic modifications of the respective reagents or starting compounds.

In some embodiments, provided herein is a compound disclosed herein, or a pharmaceutically acceptable salt thereof, or a stereoisomer or a mixture of stereoisomers thereof, or an isotopic variant thereof (e.g., including but not limited to ¹H, ²H, ³H, ¹¹C, ¹²C, ¹³C, ¹⁴C, ¹³N, ¹⁴N, ¹⁵N, ¹⁴O, ¹⁵O, ¹⁶O, ¹⁷O, ¹⁸O, ¹⁹O, ¹⁷F, ¹⁸F, ¹⁹F, ³²S, ³³S, ³⁵S, ³⁶S, ³⁷S, or ³¹S). In some embodiments, an isotopic variant is a neutron-emitter. In some embodiments, an isotopic variant is an alpha-emitter. In some embodiments, an isotopic variant is a beta-emitter. In some embodiments, an isotopic variant is a positron-emitter (beta-plus decay). In some embodiments, an isotopic variant is an electron-emitter (beta-minus decay). In some embodiments, an isotopic variant is a positron emitter. In some embodiments, provided herein is a method of detecting, observing, identifying, analyzing, or otherwise evaluating a tumor using positron emission tomography (PET), comprising administering a compound, or a pharmaceutically acceptable salt thereof, or a stereoisomer or a mixture of stereoisomers, or an isotopic variant thereof (e.g., a positron-emitter) to a subject that is to be evaluated.

In some embodiments, the compounds disclosed herein possess one or more stereocenters and each stereocenter exists independently in either the R or S configuration. In some embodiments, the conjugate described herein exists in the R configuration. In some embodiments, the conjugate described herein exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formula (I) are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

### Prodrugs

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In some embodiments, a novel class of CDK9 inhibitor prodrugs disclosed herein is to increase the therapeutic window of CDK9 inhibitors and achieve a tumor targeting of this class of anti-tumor compounds. The inventive prodrug conjugates consist of an αᵥβ3 integrin binding moiety which is linked to a CDK9 inhibitor via a peptide moiety, which is cleavable by proteases present in the tumor microenvironment to release the parent CDK9 inhibitor compound at the site of action. Enzymes present in the tumor microenvironment include, but not limited to, neutrophil elastase, and cathepsins such as cathepsin B and legumain.

Prodrugs of the compounds described herein include, but not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (e.g., the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a conjugate described herein as set forth herein are included within the scope of the claims. In some cases, some of the herein-described compounds is a prodrug for another derivative or active compound.

In some embodiments, any one of the hydroxyl group(s), amino group(s), or carboxylic acid group(s) are functionalized in a suitable manner to provide a prodrug moiety. In some embodiments, the prodrug moiety is as described above.

### Metabolite

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

### Pharmaceutical Compositions

The present invention provides a pharmaceutical composition comprising a compound of formulae disclosed herein, or a pharmaceutically acceptable salt thereof, or a stereoisomer or mixture of stereoisomers thereof; and a pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated to provide slow or controlled release of the active ingredient therein. All formulations for oral administration should be in dosages suitable for such administration. The push-fit capsules can contain the active ingredients in admixture with filler such as lactose, binders such as starches, or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

### Compounds for Cancer Treatment

In some embodiments, the present invention provides compounds of formulae disclosed herein, or a pharmaceutically acceptable salt thereof; or a stereoisomer or mixture of stereoisomers thereof, for the treatment of a disease or disorder. In some embodiments, the disease or disorder is a hyperproliferative disorder. In some embodiments, the disease or disorder is an autoimmune disorder.

In some embodiments, the disease or disorder is a cancer. In some embodiments, the cancer is a solid tumor. In some embodiments, the disease or disorder (e.g., cancer) is a hematological malignancy. In some embodiments, the disease or disorder (e.g., cancer (e.g., hematological malignancy)) is a B-cell malignancy. In some embodiments, the disease or disorder (e.g., cancer) is a MYC-driven cancer. In some embodiments, the disease or disorder (e.g., cancer) is a MCL1-driven cancer. In some embodiments, the disease or disorder (e.g., cancer) is a tumor overexpressing MYC, MYB or MCL1 mRNA, or MYC or MCL1 proteins associated therewith. In some embodiments, the disease or disorder (e.g., cancer) is a transcriptionally addicted tumor.

In some embodiments, the disease or disorder is a cancer, wherein the cancer is aggressive non-Hodgkin lymphoma (NHL), double-hit diffuse large B-cell lymphoma (DH-DLBCL), high grade B-cell lymphoma (HGBCL), transformed follicular lymphoma (FL), mantle cell lymphoma (MCL), chronic lymphocytic leukemia (CLL), small lymphocytic lymphoma (SLL), or Richter syndrome (RS). In some embodiments, the disease or disorder is a cancer, wherein the cancer is relapsed/refractory (r/r) aggressive non-Hodgkin lymphoma (r/r NHL), relapsed/refractory double-hit diffuse large B-cell lymphoma (r/r DH-DLBCL), relapsed/refractory high grade B-cell lymphoma (r/r HGBCL), relapsed/refractory transformed follicular lymphoma (r/r FL), relapsed/refractory mantle cell lymphoma (r/r MCL), relapsed/refractory chronic lymphocytic leukemia (r/r CLL), relapsed/refractory small lymphocytic lymphoma (r/r SLL), or relapsed/refractory Richter syndrome (r/r RS).

In some embodiments, the disease or disorder (e.g., cancer) is ovarian cancer, breast cancer, or prostate cancer. In some embodiments, the disease or disorder (e.g., cancer) is advanced ovarian cancer, triple negative breast cancer, or castration-resistant neuroendocrine prostate cancer. In some embodiments, the disease or disorder (e.g., cancer) is neuroblastoma. In some embodiments, the disease or disorder (e.g., cancer) is osteosarcoma. In some embodiments, the disease or disorder (e.g., cancer) is melanoma. In some embodiments, the disease or disorder is an ophthalmic condition. In some embodiments, the disease or disorder (e.g., ophthalmic condition) is macular degeneration. In some embodiments, the disease or disorder (e.g., ophthalmic condition or cancer) is uveal melanoma. In some embodiments, the disease or disorder is cardiovascular condition. In some embodiments, the disease or disorder (e.g., cardiovascular condition) is cardiac hypertrophy.

### Methods of Dosing and Treatment Regimens

The present invention relates to a method for using the compounds and compositions thereof and to treat mammalian hyper-proliferative disorders. This method comprises administering to a mammal in need thereof, including a human, an amount of the compound, which is effective to treat the disorder. Hyper-proliferative disorders include, but not limited to, solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but not limited to, invasive ductal carcinoma, invasive lobular carcinoma, ductal carcinoma in situ, and lobular carcinoma in situ.

Examples of cancers of the respiratory tract include, but, not limited to, small-cell and non-small-cell lung carcinoma, as well as bronchial adenoma and pleuropulmonary blastoma.

Examples of brain cancers include, but not limited to, brain stem and hypothalmic glioma, cerebellar and cerebral astrocytoma, medulloblastoma, ependymoma as well as neuroectodermal and pineal tumor.

Tumors of the male reproductive organs include, but not limited to, prostate and testicular cancer. Tumors of the female reproductive organs include, but not limited to, endometrial, cervical, ovarian, vaginal, and vulvar cancer, as well as sarcoma of the uterus.

Tumors of the digestive tract include, but not limited to, anal, colon, colorectal, esophageal, gallbladder, gastric, pancreatic, rectal, small intestine, and salivary gland cancers.

Tumors of the urinary tract include, but not limited to, bladder, penile, kidney, renal pelvis, ureter, and urethral cancers.

Eye cancers include, but not limited to, intraocular melanoma and retinoblastoma.

Examples of liver cancers include, but not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but not limited to, squamous cell carcinoma, Kaposi's sarcoma, malignant melanoma, Merkel cell skin cancer, and non-melanoma skin cancer.

Head-and-neck cancers include, but not limited to, laryngeal/hypopharyngeal/nasopharyngeal/oropharyngeal cancer, and lip and oral cavity cancer.

Lymphomas include, but not limited to, AIDS-related lymphoma, non-Hodgkin's lymphoma, cutaneous T-cell lymphoma, Hodgkin's disease, and lymphoma of the central nervous system.

Sarcomas include, but not limited to, sarcoma of the soft tissue, osteosarcoma, malignant fibrous histiocytoma, lymphosarcoma, and rhabdomyosarcoma.

Leukemias include, but not limited to, acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals and can be treated by administering pharmaceutical compositions of the present invention.

Based upon existing standard laboratory techniques to evaluate compounds useful for the treatment of hyper-proliferative disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

In some embodiments, the present invention provides a method of treating a disease or disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a disease or disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a hyperproliferative disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating an autoimmune disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer in a subject, comprising administering a therapeutically effective amount of a compound of formulae disclosed herein, or a pharmaceutically acceptable salt thereof, or a stereoisomer or mixture of stereoisomers thereof; or a pharmaceutical composition thereof, to an individual in need thereof.

In some embodiments, the present invention provides a method of treating a solid cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a MYC, MYB, or MCL1-driven cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a hematological malignancy in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a B-cell malignancy in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a MYC-driven cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a MCL1-driven cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a tumor overexpressing MYC, MYB or MCL1 mRNA, or MYC or MCL1 proteins associated therewith in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a tumor overexpressing MYC, MYB or MCL1 mRNA, or MYC or MCL1 proteins associated therewith in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a transcriptionally addicted tumor in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating aggressive non-Hodgkin lymphoma (NHL), double-hit diffuse large B-cell lymphoma (DH-DLBCL), high grade B-cell lymphoma (HGBCL), transformed follicular lymphoma (FL), mantle cell lymphoma (MCL), chronic lymphocytic leukemia (CLL), small lymphocytic lymphoma (SLL), or Richter syndrome (RS) in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating relapsed/refractory (r/r) aggressive non-Hodgkin lymphoma (r/r NHL), relapsed/refractory double-hit diffuse large B-cell lymphoma (r/r DH-DLBCL), relapsed/refractory high grade B-cell lymphoma (r/r HGBCL), relapsed/refractory transformed follicular lymphoma (r/r FL), relapsed/refractory mantle cell lymphoma (r/r MCL), relapsed/refractory chronic lymphocytic leukemia (r/r CLL), relapsed/refractory small lymphocytic lymphoma (r/r SLL), or relapsed/refractory Richter syndrome (r/r RS) in a subject, comprising administering a therapeutically effective amount of a compound of formulae disclosed herein, or a pharmaceutically acceptable salt thereof; or a stereoisomer or mixture of stereoisomers thereof, or a pharmaceutical composition thereof, to an individual in need thereof.

In some embodiments, the present invention provides a method of treating ovarian cancer, breast cancer, or prostate cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating advanced ovarian cancer, triple negative breast cancer, or castration-resistant neuroendocrine prostate cancer in a subject, comprising administering a therapeutically effective amount of a compound of formulae disclosed herein, or a pharmaceutically acceptable salt thereof, or a stereoisomer or mixture of stereoisomers thereof; or a pharmaceutical composition thereof, to an individual in need thereof.

In some embodiments, the present invention provides a method of treating neuroblastoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating osteosarcoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating melanoma in a subject, comprising administering a therapeutically effective amount of a compound of formulae disclosed herein, or a pharmaceutically acceptable salt thereof, or a stereoisomer or mixture of stereoisomers thereof; or a pharmaceutical composition thereof, to an individual in need thereof.

In some embodiments, the present invention provides a method of treating an ophthalmic condition in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating macular degeneration in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cardiovascular condition in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating cardiac hypertrophy in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

The total amount of the active compound to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

The present invention further provides the use of the compound of the invention for the preparation of a pharmaceutical compositions for the treatment of the aforesaid disorders.

### Administration

It is possible for the compounds according to the invention to have systemic or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly or in a modified manner, such as, for example, tablets (e.g., uncoated or coated tablets, for example with enteric or controlled release coatings that dissolve with a delay or are insoluble), orally-disintegrating tablets, films/wafers, films/lyophilisates, capsules (e.g., hard or soft gelatine capsules), sugar-coated tablets, granules, pellets, powders, emulsions, suspensions, aerosols or solutions. It is possible to incorporate the compounds according to the invention in crystalline or amorphised or dissolved form into said dosage forms.

Parenteral administration can be affected with avoidance of an absorption step (for example intravenous, intraarterial, intracardial, intraspinal or intralumbal) or with inclusion of absorption (for example, intramuscular, subcutaneous, intracutaneous, percutaneous or intraperitoneal). Administration forms which are suitable for parenteral administration are, inter alia, preparations for injection and infusion in the form of solutions, suspensions, emulsions, lyophilisates or sterile powders. In some embodiments, a compound disclosed herein is administered via injection (e.g., parenteral injection). In some embodiments, a compound disclosed herein is formulated for injection. In some embodiments, a compound disclosed herein is formulated with a pharmaceutically acceptable excipient (e.g., a carrier or vehicle, e.g., an aqueous vehicle)) for injection. In some embodiments, the injection is intravenous. In some embodiments, the intravenous injection is an intravenous infusion.

Examples which are suitable for other administration routes are pharmaceutical forms for inhalation (e.g., powder inhalers, nebulizers, etc.), nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (e.g., lotions, mixture agitandae, etc.), lipophilic suspensions, emulsions, ointments, creams, transdermal therapeutic systems (e.g., patches, etc.), milk, pastes, foams, dusting powders, implants or stents.

The compounds according to the invention can be incorporated into the stated administration forms. This can be affected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- or odour-masking agents.

The present invention furthermore relates to a pharmaceutical composition which comprise at least one compound according to the invention, conventionally together with one or more pharmaceutically suitable excipient(s), and to their use according to the present invention.

### Combination Treatments

In certain instances, it is appropriate to administer at least one conjugate described herein, or a pharmaceutically acceptable salt thereof, in combination with one or more other therapeutic agents.

In accordance with another aspect, the present invention covers pharmaceutical compositions, in particular, medicines, comprising at least one compound disclosed herein in the present invention and at least one or more further active ingredients, in particular, for the treatment or prophylaxis of a hyperproliferative disorder.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known active ingredients for the treatment and/or prophylaxis of a hyperproliferative disorder.

Examples of active ingredients for the treatment and/or prophylaxis of a hyperproliferative disorder include, but not limited to, 131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, itraconazole, ixabepilone, ixazomib, lanreotide, lansoprazole, lapatinib, Iasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polatuzumab, polyvinylpyrrolidone+sodium hyaluronate, polatuzumab vedotin, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, venetoclax, vinblastine, vincristine, vindesine, vinflunine, vinorelbine, vismodegib, vorinostat, vorozole, yttrium-90 glass microspheres, zanubrutinib, zinostatin, zinostatin stimalamer, zoledronic acid, and zorubicin.

In one embodiment, the therapeutic effectiveness of one of the compounds described herein is enhanced by administration of an adjuvant (e.g., by itself the adjuvant has minimal therapeutic benefit, but in combination with another therapeutic agent, the overall therapeutic benefit to the patient is enhanced). In some embodiments, the benefit experienced by a patient is increased by administering one of the compounds described herein with another agent (which also includes a therapeutic regimen) that also has therapeutic benefit.

In one specific embodiment, a conjugate described herein, or a pharmaceutically acceptable salt thereof, is co-administered with a second therapeutic agent, wherein the conjugate described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The conjugates described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Definitions

Unless otherwise defined, all of the technical terms used herein have the same meaning as commonly understood by one of ordinary skill in the art in the field to which this disclosure belongs.

Unless otherwise stated, the following terms used in this application have the definitions given below.

The section headings used herein are for organizational purposes only and are not to be construed as limiting the subject matter described.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, unrecited additives, components, integers, elements or method steps.

As used herein, the term “about” a number refers to that number plus or minus 10% of that number. The term ‘about’ when used in the context of a range refers to that range minus 10% of its lowest value and plus 10% of its greatest value.

Whenever the term “at least”, “greater than”, or “greater than or equal to” precedes the first numerical value in a series of two or more numerical values, the term “at least”, “greater than,” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than”, “less than”, or “less than or equal to” precedes the first numerical value in a series of two or more numerical values, the term “no more than”, “less than”, or “less than or equal to” applies to each of the numerical values in that series of numerical values. For example, less than or equal to 3, 2, or 1 is equivalent to less than or equal to 3, less than or equal to 2, or less than or equal to 1.

The use of the term “including” as well as other forms, such as “include”, “includes,” and “included,” is not limiting.

The phrase “one or more pharmaceutically acceptable excipients” is used herein to refer that one pharmaceutically acceptable excipient or more than one pharmaceutically acceptable excipient may be used in any combination. The number of pharmaceutically acceptable excipients to be used may be at the discretion of a person skilled in the art, and they may be of different types.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “specific pH” herein refers to a desired pH value of a solvent or a solution comprising CocE obtained by adding a pharmaceutically acceptable excipient.

The term “% wt” is used to describe the weight percentage of one component in a mixture of components.

The term “a trace” herein refers more than, but close to about 0%.

The term “about” herein refers to ±10%, +20%, ±30%, +40%, or ±50%, or to the nearest significant figure.

An “amino acid” refers to a natural amino acid or a non-natural amino acid. The natural amino acid is a naturally-occurring amino acid, including, but not limited to, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan. The non-natural amino acid may be incorporated into peptides for many different reasons, for instance, to increase the activity or selectivity and plasma stability of peptides or to induce or stabilize secondary structure (e.g., helices, sheets, or turns). The non-natural amino acid includes, but not limited to, N-alkyl (e.g., N-methyl) amino acids, stereoisomers (D-amino acids), homo amino acids, alpha-methyl amino acids, beta¹ amino acids, beta² amino acids, beta³ amino acids, peptoides, aminocyclohexanecarboxylate (ACHC), and non-protein amino acids, such as, citrulline, ornithine, homoalanine, norvaline, norleucine, etc. In some embodiment, the non-natural amino acid includes N-methyl alanine and citrulline.

As used herein, C₁-Cₓ includes C₁-C₂, C₁-C₃ . . . C₁-Cₓ. By way of example only, a group designated as “C₁-C₆” indicates that there are one to six carbon atoms in the moiety, e.g., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, e.g., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, e.g. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

An “hydroxyalkyl” refers to an alkyl in which one hydrogen atom is replaced by a hydroxyl. In some embodiments, a hydroxyalkyl is a C₁-C₄hydroxyalkyl. Typical hydroxyalkyl groups include, but not limited to, —CH₂OH, —CH₂CH₂OH, —CH₂CH₂CH₂OH, —CH₂CH₂CH₂CH₂OH, and the like.

An “aminoalkyl” refers to an alkyl in which one hydrogen atom is replaced by an amino. In some embodiments, aminoalkyl is a C₁-C₄aminoalkyl. Typical aminoalkyl groups include, but not limited to, —CH₂NH₂, —CH₂CH₂NH₂, —CH₂CH₂CH₂NH₂, —CH₂CH₂CH₂CH₂NH₂, and the like.

The term “alkenyl” refers to a type of alkyl group in which at least one carbon-carbon double bond is present. In one embodiment, an alkenyl group has the formula —C(R)=CR₂, wherein R refers to the remaining portions of the alkenyl group, which may be the same or different. In some embodiments, R is H or an alkyl. In some embodiments, an alkenyl is selected from ethenyl (or vinyl), propenyl (or allyl), butenyl, pentenyl, pentadienyl, and the like. Non-limiting examples of an alkenyl group include —CH═CH₂, —C(CH₃)=CH₂, —CH═CHCH₃, —C(CH₃)=CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term, “heteroalkyl” generally refers to a straight-chain and/or branched hydrocarbon chain which has 1 to 30 carbon atoms and may be interrupted once or more than once by one or more of the groups —O—, —S—, —C(═O)—, —S(═O)—, —S(═O)₂—, —NR^{y}—, —NR^{y}C(═O)—, —C(═O)—NR^{y}—, —NR^{y}NR^{y}—, —S(═O)₂—NR^{y}NR^{y}—, —C(═O)—NR^{y}NR^{y}—, —CR^{x}=N—O—, and where the hydrocarbon chain including the side chains, if present, may be substituted by —NH—C(═O)—NH₂, —C(═O)—OH, —OH, —NH₂, —NH—C(═NNH₂)—, sulfonamide, sulfone, sulfoxide, sulfonic acid, sulfamide, or a combination thereof. In this context, R^{y} in each case is —H, phenyl, C₁-C₁₀-alkyl, C₂-C₁₀-alkenyl or C₂-C₁₀-alkynyl, which may in turn be substituted in each case by —NHC(O)NH₂, —COOH, —OH, —NH₂, —NH—C(═NNH₂)—, sulfonamide, sulfone, sulfoxide, sulfonic acid, sulfamide, or a combination thereof. In this context, R^{x} is —H, C₁-C₃-alkyl or phenyl. The terms “heteroalkyl-aryl” and “heteroalkyl-heteroaryl” as used herein generally refer to a heteroalkyl group (as defined above) substituted with an aromatic carbocycle or an aromatic heterocycle respectively; and wherein each is optionally substituted.

The term “aromatic” refers to a planar ring having a delocalized π-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (or rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (or an arylene group). As used herein, the term “aralkyl” generally refers to a monocyclic aromatic carbocycle (e.g., phenyl), to which a C₁₋₄-alkyl group is bonded. Illustrative aralkyl groups include benzyl and ethylphenyl.

The term “cycloalkyl” refers to a monocyclic or polycyclic aliphatic, non-aromatic radical, wherein each of the atoms forming the ring (or skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl. In some embodiments, a cycloalkyl is a C₃-C₄cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl. The term “heteroaralkyl” as used herein generally refers to a lower alkyl group (e.g., C₁₋₆ alkyl) that is substituted with a heteraryl group. Illustrative examples include —CH₂-pyridine, —CH₂CH₂-triazole, etc.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In some embodiments, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

In some embodiments, each substituted alkyl, substituted fluoroalkyl, substituted heteroalkyl, substituted carbocycle, and substituted heterocycle is substituted with one or more R⁵ groups independently selected from the group consisting of halogen, C₁-C₆alkyl, monocyclic carbocycle, monocyclic heterocycle, —CN, —OR²¹, —CO₂R²1, —C(═O)N(R²¹)₂, —N(R²¹)₂, —NR²¹C(═O)R²², —SR²¹, —S(═O)R²², —SO₂R²², or —SO₂N(R²¹)₂; each R²¹ is independently selected from hydrogen, C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl; or two R²¹ groups are taken together with the N atom to which they are attached to form a N-containing heterocycle; each R²² is independently selected from C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl.

“Small molecule,” as used herein, refers to any molecule having a molecular weight of about 1000 kDa or less. In some embodiments, a moiety within a compound described herein is referred to as a small molecule, meaning that moiety has a molecular weight of about 1000 kDa or less. Small molecules, as used herein, excludes proteins or antibodies, but may comprise peptides or amino acids. In some instances, a compound described herein is a conjugate of two small molecule moieties. Therefore, compounds described herein may be referred to as “small molecule drug conjugates” (SMDCs) or “small molecule prodrug conjugates” (SMPCs). For example, a SMPC may contain a cleavable group (e.g., enzymatically or chemically cleavable) such as an ester which, upon cleavage, results in a SMDC. In some examples, an SMPC (sometimes referred to as a prodrug) described herein can first be cleaved (e.g., in plasma) before an enzyme can efficiently recognize and cleave the SMDC, thus liberating the active agent(s) in two steps. In some embodiments, a SMPC enables slow conversion to the SMDC, which is cleaved more rapidly (i.e., in the presence of a suitable enzyme (e.g., a tumor associated enzyme such as elastase, legumain, or cathepsin)), thus enhancing the therapeutic window or reducing side effects.

“Pharmaceutically acceptable,” as used herein, refers a material, such as a carrier or diluent, which does not abrogate the biological activity or properties of the compound, and is relatively nontoxic at the concentration or amount used, e.g., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but not limited to, the interactions of an agonist, partial agonist, an inverse agonist, antagonist, degrader, or combinations thereof. In some embodiments, a modulator is an antagonist. In some embodiments, a modulator is an inhibitor.

The terms “administer,” “administering”, “administration,” and the like, as used herein, refer to the methods that may be used to enable delivery of compounds or compositions to the desired site of biological action. These methods include, but not limited to, oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a conjugate described herein, or a pharmaceutically acceptable salt thereof, and a co-agent, are both administered to a patient simultaneously in the form of a single entity or dosage. The term “non-fixed combination” means that the active ingredients, e.g. a conjugate described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating”, or “treatment,” as used herein, include alleviating, abating or ameliorating at least one symptom of a disease or condition, preventing additional symptoms, inhibiting the disease or condition, e.g., arresting the development or progression of the disease or condition, relieving the disease or condition, causing regression of the disease or condition, relieving a secondary condition caused by the disease or condition, or stopping the symptoms of the disease or condition either prophylactically or therapeutically.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

### EXAMPLES

As used above, and throughout the description of the invention, the following abbreviations, unless otherwise indicated, shall be understood to have the following meanings:

### Abbreviations

786-0 human tumour cell line
A431NS human tumour cell line
A549 human tumour cell line
ABCBl ATP-binding cassette sub-family B member I (synonym for P-gp and MDRl)
abs. absolute
Abu α(alpha)-amino butyric acid
Ac acetyl
ACN acetonitrile
ADC antibody drug conjugate
A2DC antibody 2 drug conjugate
aq. aqueous, aqueous solution
ATP adenosine triphosphate
BCRP breast cancer resistance protein, an efflux transporter
BEP 2-bromo-1-ethylpyridinium tetrafluoroborate
Boc tert-butoxycarbonyl
br. broad (in NMR)
Ex. Example
BxPC3 human tumour cell line
ca. circa, about
C-DAR cysteine drug to antibody ratio (linker attached to cystein residues)
Cl chemical ionization (in MS)
D doublet (in NMR)
D day(s)
TLC thin-layer chromatography
DCI direct chemical ionization (in MS)
DCM dichloromethane
Dd doublet of doublets (in NMR)
DIEA N, N diisopropyl ethyl amine (Hünig's base)
DMAP 4-N,N-dimethylaminopyridine
DME 1,2-dimethoxyethane
DMEM Dulbecco's modified eagle medium (standardized nutrient medium for cell culture)
DMF N,N-dimethylformamide
DMSO dimethyl sulphoxide
DAR drug to antibody ratio
DPBS Dulbecco's phosphate-buffered salt solution
Dt doublet of triplets (in NMR)
DTT DL-dithiothreitol
d. Th. of theory (in chemical yield)
EDC N′-(3-dimethylaminopropyl)-N-ethylcarbodi-imide hydrochloride
EGFR epidermal growth factor receptor
ee: enantiomeric excess
EI electron impact ionization (in MS)
ELISA enzyme-linked immunosorbent assay
eq. equivalent(s)
ESI electrospray ionization (in MS)
FCS foetal calf serum
Fmoc (9H-fluoren-9-ylmethoxy)carbonyl
sat. saturated
GTP guanosine-5′-triphosphate
h hour(s)
HATU O-(7-azabenzotriazol-1-yl)-N,N,N′,N′-tetramethyluronium hexafluoro phosphate
HCT-116 human tumour cell line
HEPES 4-(2-hydroxyethyl)piperazine-1-ethane-sulphonic acid
HOAc acetic acid
HOAt 1-hydroxy-7-azabenzotriazole
HOBt 1-hydroxy-1H-benzotriazole hydrate
HOSu N-hydroxysuccinimide
HPLC high-pressure, high-performance liquid chromatography
HT29 human tumour cell line
IC50 half-maximal inhibitory concentration
i.m. intramuscularly, administration into the muscle
i.v. intravenously, administration into the vein
K-DAR lysine drug to antibody ratio (linker attached to lysine residues)
KPL-4 human tumour cell lines
KU-19-19 human tumour cell line
LC-MS liquid chromatography-coupled mass spectrometry
LLC-PKl cells Lewis lung carcinoma pork kidney cell line
L-MDR human MDRl transfected LLC-PKl cells
LoVo human tumour cell line
m multiplet (in NMR)
Me methyl
MDRl Multidrug resistance protein I
MeCN acetonitrile
min minute(s)
MS mass spectrometry
MTBE methyl tert.-butyl ether
MTT 3-(4,5-dimethylthiazol-2-yl)-2,5-diphenyl-2H-tetrazolium bromide
NCI-H292 human tumour cell line
NCI-H520 human tumour cell line
NMM N-methylmorpholine
NMP N-methyl-2-pyrrolidinone
NMR nuclear magnetic resonance spectrometry
NMRI mouse strain originating from the Naval Medical Research Institute
Nude mice experimental animals
NSCLC non small cell lung cancer
PBS phosphate-buffered salt solution
Pd/C palladium on activated carbon
P-gp P-glycoprotein, a transporter protein
PNGaseF enzyme for cleaving sugar
Quant quantitative (in yield)
quart quartet (in NMR)
quint quintet (in NMR)
RT room temperature
Rt retention time (in HPLC)
s singlet (in NMR)
s.c. subcutaneously, administration under the skin
SCC-4 human tumour cell line
SCC-9 human tumour cell line
SCID mice test mice with severe combined immunodeficiency
SK-HEP-I human tumour cell line
t triplet (in NMR)
T3P® 2,4,6-tripropyl-1,3,5,2,4,6-trioxatriphosphi-nane 2,4,6-trioxide
TBAF tetra-n-butylammonium fluoride
TCEP tris(2-carboxyethyl)phosphine
TEMPO (2,2,6,6-tetramethylpiperidin-1-yl)oxyl
tert tertiary
TFA trifluoroacetic acid
THE tetrahydrofuran
TLC thin-layer chromatography
UV ultraviolet spectrometry
v/v volume to volume ratio (of a solution)
Z benzyloxycarbonyl

The following examples are provided for illustrative purposes only and not to limit the scope of the claims provided herein.

### ANALYTICAL EXAMPLES

### Example A1: Liquid Chromatography-Mass Spectrometry (LC-MS)

Method 1 (LC-MS): System MS: Thermo Scientific FT-MS; System UHPLC+: Thermo Scientific UltiMate 3000; Column: Waters, HSST3, 2.1×75 mm, C18 1.8 μm; Eluent A: 1 l Water+0.01% Formic acid; Eluent B: 1 l Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm/Optimum Integration Path 210-300 nm

Method 2 (LC-MS): System MS: Thermo Scientific FT-MS; System UHPLC+: Thermo Scientific Vanquish; Column: Waters, HSST3, 2.1×75 mm, C18 1.8 μm; Eluent A: 1 l Water+0.01% Formic acid; Eluent B: 1 l Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm

Method 3 (LC-MS): System MS: Waters TOF instrument; System UPLC: Waters Acquity I-CLASS; Column: Waters, HSST3, 2.1×50 mm, C18 1.8 μm; Eluent A: 1 l Water+0.01% Formic acid; Eluent B: 1 l Acetonitrile+0.01% Formic acid; Gradient: 0.0 min 2% B 0.5 min 2% B→7.5 min 95% B→10.0 min 95% B; Oven: 50° C.; Flow: 1.00 ml/min; UV-Detection: 210 nm

Method 4 (LC-MS): Instrument: Waters ACQUITY SQD UPLC System; Column: Waters Acquity UPLC HSS T3 1.8 μm 50×1 mm; Eluent A: 1 l Water+0.25 ml 99% ige Formic acid, Eluent B: 1 l Acetonitrile+0.25 ml 99% ige Formic acid; Gradient: 0.0 min 95% A→6.0 min 5% A→7.5 min 5% A Oven: 50° C.; Flow: 0.35 ml/min; UV-Detection: 210 nm.

Method 5 (LC-MS): System MS: Waters TOF instrument; System UPLC: Waters Acquity I-CLASS; Column: Waters Acquity UPLC HSS T3 1.8 μm 50×1 mm; Eluent A: 1 l Water+0.100 ml 99% ige Formic acid, Eluent B: 1 l Acetonitrile+0.100 ml 99% ige Formic acid; Gradient: 0.0 min 90% A→1.2 min 5% A→2.0 min 5% A Oven: 50° C.; Flow: 0.40 ml/min; UV-Detection: 210 nm.

### SYNTHESIS EXAMPLES

### Example B1: Preparation of (3R)-3-{[(4-{[(4-nitrophenoxy)carbonyl]amino}phenyl)carbamoyl]amino}-3-{3-[({3-[(propylcarbamoyl)amino]phenyl}sulfonyl)amino]phenyl}propanoic acid (Building block 1)

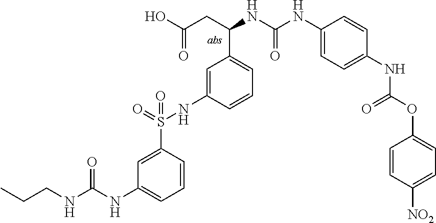 [SMILES: CCCNC(=O)Nc1cccc(S(=O)(=O)Nc2cccc([C@@H](CC(=O)O)NC(=O)Nc3ccc(NC(=O)Oc4ccc([N+](=O)[O-])cc4)cc3)c2)c1]

The synthesis of Building block 1 has been described in WO2020/094471. LC-MS: Rt=0.89 min; MS (ESIpos): m/z=720 [M+H]⁺.

### Example B2: Preparation of (3S)-3-{[(4-{[(4-nitrophenoxy)carbonyl]amino}phenyl)carbamoyl]amino}-3-{3-[({3-[(propylcarbamoyl)amino]phenyl}sulfonyl)amino]phenyl}propanoic acid (Building block 2)

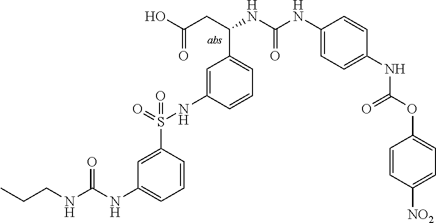 [SMILES: CCCNC(=O)Nc1cccc(S(=O)(=O)Nc2cccc([C@H](CC(=O)O)NC(=O)Nc3ccc(NC(=O)Oc4ccc([N+](=O)[O-])cc4)cc3)c2)c1]

The synthesis of building block 2 has been described in WO2020/094471. LC-MS: Rt=0.89 min; MS (ESIpos): m/z=720 [M+H]⁺.

### Example B3: Preparation of (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (Building block 3)

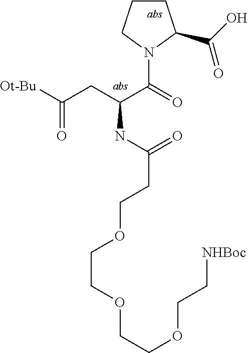 [SMILES: CC(C)(C)OC(=O)C[C@H](NC(=O)CCOCCOCCOCCNC(=O)OC(C)(C)C)C(=O)N1CCC[C@H]1C(=O)O]

Building block 3 was synthesized using classical methods of peptide synthesis starting with the coupling of Z-Asp(OtBu)-OH with benzyl L-prolinate hydrochloride (1:1) in THE in the presence of T3P and DIPEA and subsequent removal of the Z-protecting group as well as the benzyl ester by hydrogenolysis over Pd/C to give (2S)-1-[(2S)-2-amino-4-tert-butoxy-4-oxobutanoyl] pyrrolidine-2-carboxylic acid. This partially protected dipeptide was acylated with tert-butyl{2-[2-(2-{3-[(2,5-dioxopyrrolidin-1-yl)oxy]-3-oxopropoxy}ethoxy)ethoxy]ethyl}carbamate to give the title compound. Tert-butyl{2-[2-(2-{3-[(2,5-dioxopyrrolidin-1-yl)oxy]-3-oxopropoxy}ethoxy) ethoxy]ethyl}carbamate was previously prepared by reacting 2,2-dimethyl-4-oxo-3,8,11,14-tetraoxa-5-azaheptadecan-17-oic acid with N-Hydroxysuccinimide in dioxane in the presence of EDCI. LC-MS: Rₜ=0.81 min; MS (ESIpos): m/z=590 [M+H]⁺.

### Example B4. Preparation of 4-(4-fluoro-2-methoxyphenyl)-N-{3-[(R-methylsulfonimidoyl) methyl]phenyl}-1,3,5-triazin-2-amine (Building bock 4 (PT-1″))

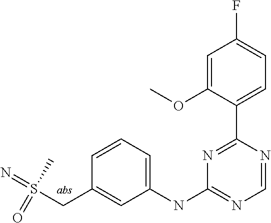 [SMILES: COc1cc(F)ccc1-c1ncnc(Nc2cccc(C[S@](C)(=N)=O)c2)n1]

The synthesis of Building block 4 was described in ChemMedChem (2017), 12(21), 1776-1793.

### Example B5. Preparation of (S)-5-Fluoro-4-(4-fluoro-2-methoxyphenyl)-N-{4-[(S-methylsulfonimidoyl)methyl]pyridin-2-yl}pyridin-2-amine (Building block 5 (PT-2′))

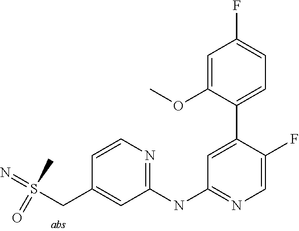 [SMILES: COc1cc(F)ccc1-c1cc(Nc2cc(C[S@@](C)(=N)=O)ccn2)ncc1F]

Building block 5 was synthesized as described in J. Med. Chem. (2021), 64(15), 11651-11674. (S)-5-Fluoro-4-(4-fluoro-2-methoxyphenyl)-N-{4-[(S-methylsulfonimidoyl) methyl]pyridin-2-yl}pyridin-2-amine (3.468 g) Building block 5 (PT-2′) was purified via chiral preparative HPLC [a Sepiatec Prep SFC 100 system with a Chiralpak IC 5 m, 250×30 mm column. Eluent: CO₂/i-PrOH (+0.4% diethylamine) 7:3; flow: 100 mL/min; pressure (outlet): 150 bar; temperature: 40° C.; solution: 3.468 g in 55 mL DCM/MeOH 2:1; detection: UV 254 nm] to give (R)-5-Fluoro-4-(4-fluoro-2-methoxyphenyl)-N-{4-[(S-methylsulfonimidoyl)methyl]pyridin-2-yl}pyridin-2-amine (PT-2″) and Building block 5 (PT-2′). Yield: 1.32 g, 3.26 mmol. HPLC Rt=8.5-10.5 min. ESI-HRMS: m/z [M+H]⁺ calcd for C₁₉H₁₉F₂N₄O₂S: 405.1198, found: 405.1196. [α]_{D}+11.4±0.13 (c 1.00, DMSO).

### Example B6. Preparation of (S)-((1⁴,2⁵-difluoro-5,10-dioxa-3-aza-2(4,2)-pyrimidina-1(1,2),4(1,3)-dibenzenacyclodecaphane-4⁵-yl)methyl)(imino)(methyl)-λ⁶-sulfanone (Building block 6 (PT-3′))

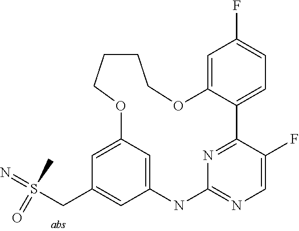 [SMILES: C[S@](=N)(=O)Cc1cc2cc(c1)OCCCCOc1cc(F)ccc1-c1nc(ncc1F)N2]

Building block 6 (PT-3′) was synthesized as described in WO2015/155197 and purified via chiral preparative HPLC to give the title compound as a single stereoisomer.

Example B7. Preparation of (4R or S*)-15,19-difluoro-8-[(R or S*-methanesulfonimidoyl)methyl]-4-methyl-3,4-dihydro-2H,11H-12,16-(azeno)-10,6-(metheno)-1,5,11,13-benzodioxadiazacyclooctadecine (single diastereomer) (Building block 7 (PT-4))

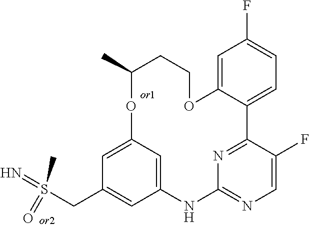 [SMILES: C[C@H]1CCOc2cc(F)ccc2-c2nc(ncc2F)Nc2cc(C[S@@](C)(=N)=O)cc(c2)O1]

Building block 7 was prepared from commercially available intermediates according to the following steps.

Step 1: Synthesis of 2-chloro-5-fluoro-4-(4-fluoro-2-methoxyphenyl)pyrimidine (B7-1)

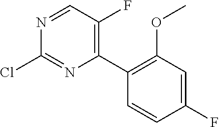 [SMILES: COc1cc(F)ccc1-c1nc(Cl)ncc1F]

Commercially available 2,4-dichloro-5-fluoropyrimidine (100 g, 599 mmol) and (4-fluoro-2-methoxyphenyl)boronic acid (112 g, 659 mmol) were dissolved in 1,2-dimethoxyethane (1797 mL). A 2M K₂CO₃ solution (898 mL) was added, and the reaction was stirred for 10 min at rt under argon. The catalyst, Pd(dppf)C₁₂ (24.5 g, 29.9 mmol), was added and stirring was continued for 2 h at 90° C. The reaction was allowed to cool down to rt and was diluted with ethyl acetate (2000 mL). The organic solution was washed with sat. NaCl solution (500 mL), dried over Na₂SO₄, filtered, and concentrated in vacuo. The crude product was dissolved in DCM and purified by column chromatography (10-100% hexane in DCM), which gave B7-1 (52.5 g, 204.6 mmol, 34%).

Step 2: Synthesis of 2-(2-chloro-5-fluoropyrimidin-4-yl)-5-fluorophenol (B7-2)

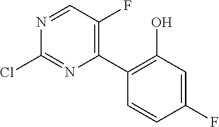 [SMILES: Oc1cc(F)ccc1-c1nc(Cl)ncc1F]

B7-1 (10 g, 39.0 mmol) was dissolved in DCM (100 mL) and cooled to 0° C. A 1M boron tribromide solution (108.3 mL, 108.3 mmol) was added dropwise and the reaction was stirred overnight allowing the solution to come to rt. The reaction was cooled to 0° C. again and sat. NaHCO₃ solution (200 mL) was added in batches. Stirring at rt was continued for 1 h. The layers were separated, and the aqueous phase was extracted with DCM. The combined organic layers were extracted with NaCl solution, dried over sodium sulphate, and concentrated in vacuo at 40° C. to give B7-2 in quantitative yield (9.46 g, 39.0 mmol, 100%).

Step 3: Synthesis of 3-(chloromethyl)-5-nitrophenol (B7-3)

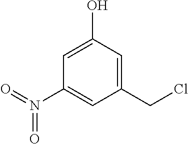 [SMILES: O=[N+]([O-])c1cc(O)cc(CCl)c1]

3-(Hydroxymethyl)-5-nitrophenol (200 g, 1.18 mol) was dissolved in DMF (2000 mL) at 0° C. and thionyl chloride (170 mL, 2.4 mol) was dripped into the solution. The reaction was stirred for 3 h at 10° C. and overnight at rt. The reaction was concentrated in vacuo, followed by the addition of water (2500 mL). This step required additional cooling due to the exothermic reaction. The aqueous solution was extracted with diethyl ether (3×1000 mL) and the combined organic phases were washed with saturated NaCl (1000 mL), dried and concentrated in vacuo to give B7-3 in quantitative yield (222 g, 1.18 mmol, 100%).

Step 4: Synthesis of 3-[(methylsulfanyl)methyl]-5-nitrophenol (B7-4)

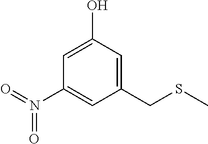

B7-3 (100.6 g, 536 mmol) was dissolved in acetone (910 mL, 12 mol). Sodium thiomethoxide solution (352 mL, 1.2 mol, 21% in water) was dripped into the solution. Due to the exothermic response, the reaction was cooled with an ice bath. The reaction was stirred overnight. Diethyl ether (700 mL) was added, and the reaction was acidified with 2M HCl. Phases were separated and the aqueous phase was extracted with diethyl ether (300 mL). The combined organic phases were washed with brine (2×300 mL), dried and concentrated in vacuo. The crude product was purified by flash column chromatography (0-20% diethyl ether in hexane) to give B7-4 (91.6 g, 459.8 mmol, 86%).

Step 5: Synthesis of 3-{3-[(methylsulfanyl)methyl]-5-nitrophenoxy}butan-1-ol (B7-5)

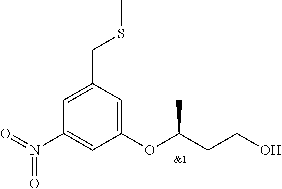 [SMILES: CSCc1cc(O[C@@H](C)CCO)cc([N+](=O)[O-])c1]

B7-4 (6.0 g, 30.1 mmol) and potassium carbonate (12.5 g, 90.3 mmol) were added to DMF (120 mL). 3-Bromobutan-1-ol (6.9 g, 45.1 mmol) was added and the reaction was stirred under nitrogen for 3 h at 100° C. Additional 3-bromobutan-1-ol (1.5 g, 9.8 mmol) was added and the reaction was stirred for a further 1 h at 100° C. Water (150 mL) was added, and the reaction was extracted with diethyl ether (3×250 mL). The organic phase was washed with saturated NaCl solution, filtered, and concentrated in vacuo. The crude product was purified by flash column chromatography (30-60% hexane in diethyl ether) to give B7-5 (7.0 g, 25.8 mmol, 86%). LC-MS: Rₜ=1.05 min; MS (ESIpos): m/z=294 [M+Na]⁺.

Step 6: Synthesis of 2-chloro-5-fluoro-4-[4-fluoro-2-(3-{3-[(methylsulfanyl)methyl]-5-nitrophenoxy}butoxy)phenyl]pyrimidine (B7-6)

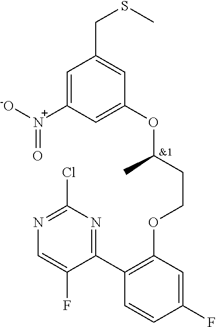

B7-5 (9.3 g, 34.3 mmol), B7-2 (7.6 g, 31.2 mmol) and triphenyl phosphine (9.8 g, 37.4 mmol) were dissolved in THE (80 mL) and cooled to 0° C. A solution of diisopropyl azodicarboxylate (DIAD) (7.6 g, 37.4 mmol) in THE (8 mL) was added in a dropwise fashion and the reaction was left to stir overnight at rt. The reaction was concentrated in vacuo and purified by flash column chromatography (0-30% diethyl ether in hexane) to give B7-6 (12.9 g, 25.7 mmol, 75%).

B7-6 was dissolved in acetone (88 mL) and the mixture of enantiomers was separated (Chiralpak IF, 4.60 mm diameter, 100.0 mm length, solvent CO₂/Methanol 87/13). The desired enantiomer (optical rotation 51.10°±0.16°, 20 C, 589 nm) was obtained with 90.3% ee at Rₜ 9.0-10.4 min and was used for subsequent reactions. LC-MS: Rₜ=1.54 min; MS (ESIpos): m/z=496 [M+H]⁺. Single (+) isomer, absolute stereochemistry unknown.

Step 7: Synthesis of rel-2-chloro-5-fluoro-4-(4-fluoro-2-{[(3R)-3-(3-{[(S)-methylsulfinyl]methyl}-5-nitrophenoxy)butyl]oxy}phenyl)pyrimidine (B7-7)

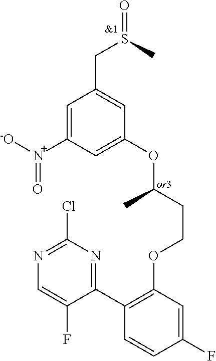

The (+)-enantiomer of B7-6 (2.79 g, 5.63 mmol) was dissolved in acetonitrile (71 mL) and cooled to 0° C. Iron trichloride (91.3 mg, 0.56 mmol) was added while stirring and stirring was continued for 15 min. Hydrogen periodate (3.85 g, 16.88 mmol) was added and stirring was continued for an additional 1.5 h with cooling. Sodium thiosulfate solution was added (200 mL) and the aqueous phase was extracted with diethyl ether (3×200 mL). The organic phase was washed with saturated sodium chloride solution, filtered, and concentrated in vacuo to give B7-7 (2.78 g, 5.38 mmol, 96%). ¹H NMR (400 MHz, DMSO-d6, 300K) δ ppm 1.24-1.35 (m, 3H), 1.92-2.16 (m, 2H), 4.03 (dd, J=12.7, 2.0 Hz, 1H), 4.18-4.28 (m, 3H), 4.62-4.75 (m, 1H), 6.97 (td, J=8.4, 2.4 Hz, 1H), 7.19 (dd, J=11.4, 2.0 Hz, 1H), 7.29 (br s, 1H), 7.50-7.60 (m, 2H), 7.76 (d, J=1.5 Hz, 1H), 8.89 (d, J=1.8 Hz, 1H). LC-MS: Rₜ=1.24 min; MS (ESIpos): m/z=512 [M+H]⁺.

Step 8: Synthesis of tert-butyl {[3-({4-[2-(2-chloro-5-fluoropyrimidin-4-yl)-5-fluorophenoxy]butan-2-yl}oxy)-5-nitrobenzyl](methyl)oxido-sulfanylidene}carbamate (B7-8)

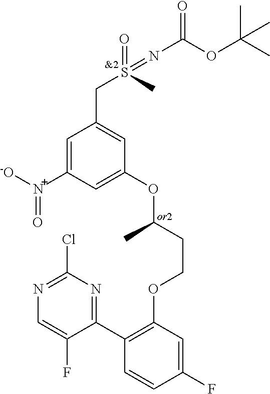

B7-7 (2.78 g, 5.43 mmol), tertbutyl carbamate (1.08 g, 9.23 mmol), magnesium oxide (875.5 mg, 21.7 mmol), rhodium(III)acetate dimer (120.0 mg, 0.27 mmol) and (diacetoxyiodo)benzene (2.62 g, 8.15 mmol) were stirred in DCM (52 mL) for 24 h at 40° C. The reaction was filtered through a thin pad of Celite™, washed with DCM and concentrated in vacuo. The crude product was purified by flash column chromatography (0-50% diethyl ether in hexane) to give B7-8 (2.93 g, 4.54 mmol, 84%).

Step 9: Synthesis of tert-butyl {[3-amino-5-({4-[2-(2-chloro-5-fluoropyrimidin-4-yl)-5-fluorophenoxy]butan-2-yl}oxy)benzyl](methyl)oxido-sulfanylidene}carbamate B7-9

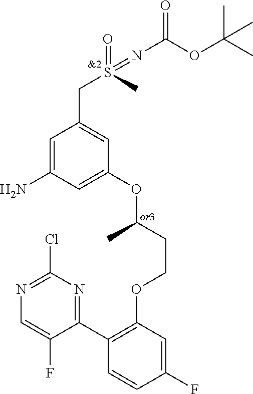 [SMILES: C[C@H](CCOc1cc(F)ccc1-c1nc(Cl)ncc1F)Oc1cc(N)cc(C[S@](C)(=O)=NC(=O)OC(C)(C)C)c1]

B7-8 (5.05 g, 8.05 mmol) was dissolved in MeOH/THF. Platinum on activated carbon (785.5 mg, 0.04 mmol) was added under nitrogen and the reaction was stirred for 3 h under H₂. More platinum on activated carbon (785.5 mg, 0.04 mmol) was added and the reaction was stirred for a further 4 h under H₂ and then overnight under nitrogen. The reaction mixture was filtered through a thin pad of Celite™, was washed with MeOH/THF and the filtrate was concentrated in vacuo to give crude B7-9 (4.59 g, 7.30 mmol, 91%).

Step 10: Synthesis of tert-butyl [{[15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxido-sulfanylidene]carbamate (B7-10)

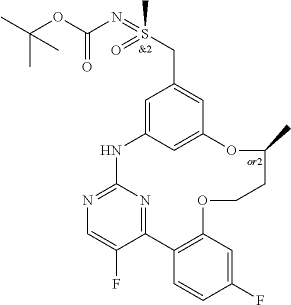 [SMILES: C[C@H]1CCOc2cc(F)ccc2-c2nc(ncc2F)Nc2cc(C[S@](C)(=O)=NC(=O)OC(C)(C)C)cc(c2)O1]

B7-9 (2.59 g, 4.34 mmol) was dissolved in toluene (259 mL) and 1-methyl-2-pyrrolidone (25.9 mL). Chloro(2-dicyclohexylphosphino-2 (358.7 mg, 0.434 mmol), Xphos (206.8 mg, 0.434 mmol) and potassium phosphate (4.60 g, 21.7 mmol) were added and the reaction was stirred for 2.5 h at 130° C. under nitrogen. Water (300 mL) was added to the reaction mixture and it was extracted with diethyl ether (3×200 mL). The organic phase was washed with saturated brine, filtered and concentrated in vacuo. The crude product was purified by flash column chromatography (0-20% diethyl ether in hexane) to give B7-10 (2.16 g, 3.86 mmol, 89%).

B7-10 was dissolved in DCM/MeOH 1:1 (20 mL) and the mixture of enantiomers was separated (Chiralpak IF, 4.60 mm diameter, 100.0 mm length, solvent CO₂/Methanol 69/31). The desired enantiomer (optical rotation 119.0°±0.34°, 20° C., 589 nm) was obtained with 96.0% ee at Rₜ=3.70 min and was used in subsequent reactions.

Step 11: Synthesis of (4R or S*)-15,19-difluoro-8-[(R or S*-methanesulfonimidoyl)methyl]-4-methyl-3,4-dihydro-2H,11H-12,16-(azeno)-10,6-(metheno)-1,5,11,13-benzodioxadiazacyclooctadecine (Building block 7 (PT-4)) (single diastereomer, absolute stereochemistry unknown)

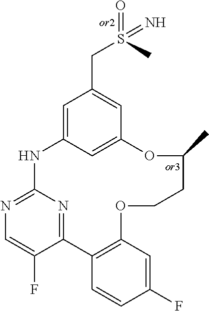 [SMILES: C[C@H]1CCOc2cc(F)ccc2-c2nc(ncc2F)Nc2cc(C[S@@](C)(=N)=O)cc(c2)O1]

B7-10 (140 mg, 0.25 mmol) was dissolved in DCM (3.26 mL). TFA (0.48 mL, 6.24 mmol) was added, and the reaction was stirred for 1.5 h at rt. The reaction was made basic with saturated NaHCO₃ solution and was extracted with DCM (3×20 mL). The organic phase was washed with saturated brine, filtered, and concentrated in vacuo. The crude product was purified by prep. HPLC to give Building block 7 (PT-4) (85 mg, 0.18 mmol, 72%). ¹H-NMR (400 MHz, DMSO-d₆): δ [ppm]=9.82 (s, 2H), 8.68 (d, 2H), 8.67-8.66 (m, 2H), 7.63 (ddd, 2H), 7.38 (d, 1H), 7.35 (d, 1H), 6.93 (t, 2H), 6.73 (t, 2H), 6.47 (dd, 2H), 5.76 (s, 1H), 4.47 (t, 2H), 4.43-4.31 (m, 2H), 4.27-4.22 (m, 2H), 4.21-4.14 (m, 2H), 4.13-4.05 (m, 2H), 3.55 (d, 2H), 2.81 (s, 6H), 2.67 (t, 1H), 2.52-2.52 (m, 1H), 2.40-2.30 (m, 3H), 1.77-1.66 (m, 2H), 1.44 (d, 6H). Single enantiomer, absolute stereochemistry unknown.

Example B8: Preparation of N-{2-[{2-[(tert-butoxycarbonyl)(methyl)amino]ethyl}(methyl)amino]ethyl}-N-methylglycine (Building block 8)

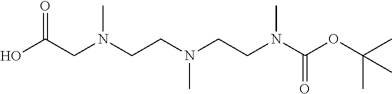 [SMILES: CN(CCN(C)CC(=O)O)CCN(C)C(=O)OC(C)(C)C]

Initially, tert-butyl methyl(2-{methyl[2-(methylamino)ethyl] amino}ethyl)carbamate was obtained starting from commercially available N,N′-dimethyl-N-[2-(methylamino)ethyl]ethane-1,2-diamine. Benzyl bromoacetate (609 mg, 2.66 mmol) was initially dissolved under argon in acetonitrile (25 mL) and potassium carbonate (734 mg, 5.31 mmol) was added. The solution was cooled to 0° C. and a solution of tert-butyl methyl(2-{methyl[2-(methylamino)ethyl] amino}ethyl)carbamate (652 mg, 2.66 mmol) in acetonitrile was added. The batch was stirred at rt for 1 h and subsequently filtered. The filtrate was evaporated in vacuo and the remaining residue was purified by prep. HPLC. Relevant fractions were collected and evaporated to dryness. 665 mg (97% purity, 62% yield) of the protected intermediate benzyl N-{2-[{2-[(tert-butoxycarbonyl)(methyl)amino]ethyl}(methyl)amino]ethyl}-N-methylglycinate were obtained as a colorless oil. This intermediate (687 mg, 1.75 mmol) was dissolved in DCM/methanol and hydrogenated over 10% Pd on charcoal at rt for 2 h. The catalyst was filtered off and the filtrate was concentrated in vacuo to give Building block 8 (555 mg, 61% purity, 64% yield) as a colorless oil. LC-MS: Rₜ=0.74 min; MS (ESIpos): m/z=304 [M+H]⁺.

Example B9: Preparation of N-{2-[{2-[(tert-butoxycarbonyl)(methyl)amino]ethyl}(methyl)amino]ethyl}-N-methylglycyl-L-asparaginyl-L-proline (Building block 9)

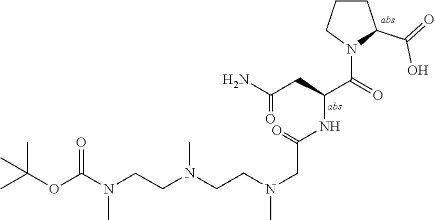 [SMILES: CN(CCN(C)CC(=O)N[C@@H](CC(N)=O)C(=O)N1CCC[C@H]1C(=O)O)CCN(C)C(=O)OC(C)(C)C]

Building block 9 was synthesized using classical peptide synthesis methods starting with the coupling of Boc-Asn with benzyl L-prolinate hydrochloride (1:1) in DMF in the presence of HATU and N,N-Diisopropylethylamine and subsequent removal of the Boc-protecting group with TFA in DCM. This partially protected dipeptide was acylated with N-{2-[{2-[(tert-butoxycarbonyl)(methyl)amino]ethyl}(methyl)amino]ethyl}-N-methylglycine (Building block 8) in DMF in the presence of HATU and N, N-Diisopropylethylamine. In the final step the benzylester was removed by hydrogenolysis over 10% Pd/charcoal. LC-MS: Rₜ=0.81 min; MS (ESIpos): m/z=515 [M+H]⁺

Example B10: Preparation of N-(tert-butoxycarbonyl)-L-alanyl-N-methyl-L-alanine (Building block 10)

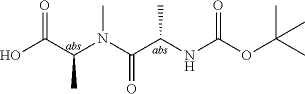 [SMILES: C[C@H](NC(=O)OC(C)(C)C)C(=O)N(C)[C@@H](C)C(=O)O]

Building block 10 was synthesized by coupling 2,5-dioxopyrrolidin-1-yl N-(tert-butoxycarbonyl)-L-alaninate to N-methyl-L-alanine in DMF in the presence of N,N-diisopropylethylamine.

Example B11: Preparation of N-methyl-N-(2,2,8,11-tetramethyl-4-oxo-3-oxa-5,8,11-triazatridecan-13-yl)glycine (Building block 11)

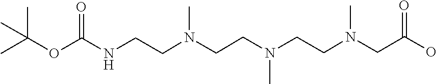

Commercially available N,N′-dimethyl-N-[2-(methylamino) ethyl]ethane-1,2-diamine (500 mg, 3.44 mmol) was dissolved under argon in acetonitrile (50 mL) and potassium carbonate (476 mg, 3.44 mmol) was added. The solution was cooled to 0° C. and a solution of benzyl bromoacetate (394 mg, 1.72 mmol) in acetonitrile was added. The reaction was stirred at rt for 20 h and was filtered. The filtrate was evaporated and the remaining residue was purified by prep. HPLC. Relevant fractions were collected and evaporated to dryness to give 476 mg (99% purity, 34% yield) of the protected intermediate as a colorless oil. This intermediate was alkylated reductively in methanol and 2.5 equivalents of acetic acid with tert-butyl (2-oxoethyl)carbamate in the presence of trihydrido(pyridine)boron. Subsequently, the benzyl ester was removed by hydrogenolysis over 10% Pd/charcoal to give Building block 11. LC-MS: Rₜ=0.47 min; MS (ESIpos): m/z=347 [M+H]⁺. ¹H NMR (600 MHz, DMSO-d₆) δ ppm 1.394 (s, 9H) 2.378 (s, 3H) 2.542 (s, 2H) 2.779 (s, 3H) 2.812 (s, 3H) 2.852-2.944 (m, 4H) 3.145 (br t, J=6.26 Hz, 2H) 3.229 (br t, J=5.87 Hz, 1H) 3.262-3.344 (m, 4H) 3.975 (s, 3H) 7.098 (br t, J=5.09 Hz, 1H)

Example B12: Preparation of N-methyl-N-(2,2,8,11-tetramethyl-4-oxo-3-oxa-5,8,11-triazatridecan-13-yl)glycyl-L-asparaginyl-L-proline (Building block 12)

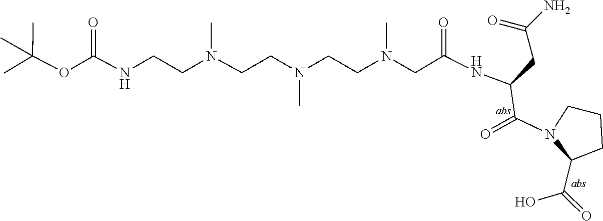 [SMILES: CN(CCNC(=O)OC(C)(C)C)CCN(C)CCN(C)CC(=O)N[C@@H](CC(N)=O)C(=O)N1CCC[C@H]1C(=O)O]

Building block 12 was synthesized using classical peptide synthesis methods starting with the coupling of Boc-Asn with benzyl L-prolinate hydrochloride (1:1) in DMF in the presence of HATU and N, N-diisopropylethylamine and subsequent removal of the Boc-protecting group with TFA in DCM to yield the dipeptide H-L-Asn-L-Pro-OBzl as the trifluoroacetate salt. This partially protected dipeptide was acylated with N-methyl-N-(2,2,8,11-tetramethyl-4-oxo-3-oxa-5,8,11-triazatridecan-13-yl)glycine (Building block 11) in DMF in the presence of HATU and N, N-diisopropylethylamine. In the final step, the benzyl ester was removed by hydrogenolysis over 10% Pd/charcoal, yielding Building block 12. LC-MS: Rₜ=0.54 min; MS (ESIpos): m/z=558 [M+H]⁺. ¹H NMR (600 MHz, DMSO-d₆) δ ppm 1.393 (s) 1.851-1.956 (m) 2.074 (s) 2.129 (br dd, J=12.13, 8.22 Hz) 2.365-2.413 (m) 2.480-2.516 (m) 2.543 (s) 2.768 (s) 2.820 (s) 2.843-2.906 (m) 3.158 (br t, J=6.06 Hz) 3.217 (br t, J=5.87 Hz) 3.284-3.347 (m) 3.383 (dd, J=8.51, 5.77 Hz) 3.613-3.654 (m) 3.793 (br d, J=13.30 Hz) 4.227 (dd, J=8.61, 4.11 Hz) 4.712-4.752 (m) 4.848-4.902 (m) 4.915-4.955 (m) 6.902 (br s) 6.966 (s) 7.103 (br t, J=5.28 Hz) 7.452 (br s) 7.472 (br s) 8.769-8.817 (m) 8.941 (br d, J=7.63 Hz).

Example B13: Preparation of (rac)-3,21-difluoro-10-[(S-methylsulfonimidoyl)methyl]-13-oxa-5,7,19,26-tetraazatetracyclo[18.3.1.1^{2,6}.1^{8,12}]hexacosa-1(24),2(26),3,5,8(25),9,11,20,22-nonaene (Building block 13)

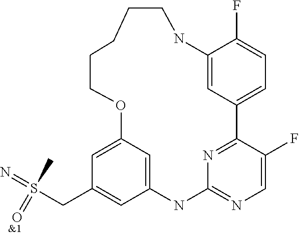 [SMILES: C[S@](=N)(=O)Cc1cc2cc(c1)OCCCCCNc1cc(ccc1F)-c1nc(ncc1F)N2]

The synthesis of Building block 13 has been described in WO2018/177889. ¹H-NMR (400 MHz, DMSO): δ=1.48-1.61 (m, 2H), 1.73-1.88 (m, 41H), 2.80-2.92 (m, 3H), 3.17-3.28 (m, 2H), 3.52-3.64 (m, 1H), 3.96-4.07 (m, 2H), 4.21-4.35 (m, 2H), 5.76-5.84 (m, 1H), 6.57-6.68 (m, 1H), 6.78-6.86 (m, 1H), 7.13-7.24 (m, 2H), 7.49-7.61 (m, 1H), 8.27-8.38 (m, 1H), 8.55-8.64 (m, 1H), 9.80-9.92 (m, 1H).

Examples B14 & B15: Preparation of (R and S)-(5,23-difluoro-8,13-dioxa-19,21,24-triazatetracyclo[18.3.1114,18.02,7]pentacosa-1(23),2,4,6,14,16,18(25),20(24),21-nonaen-16-yl)methyl-imino-methyl-oxo-λ⁶-sulfane (two single enantiomers) (Building block 14, Building block 15)

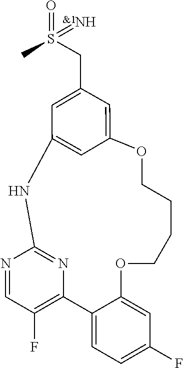 [SMILES: C[S@](=N)(=O)Cc1cc2cc(c1)OCCCCOc1cc(F)ccc1-c1nc(ncc1F)N2]

Building block 14 and Building block 15 were prepared from commercially available intermediates according to the following steps.

Step 1: Synthesis of tert-butyl-dimethyl-[4-[3-(methylsulfinylmethyl)-5-nitro-phenoxy]butoxy]silane (B14-1)

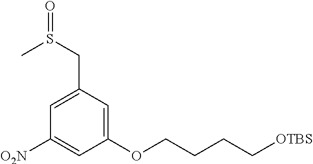 [SMILES: CS(=O)Cc1cc(OCCCCO[Si](C)(C)C(C)(C)C)cc([N+](=O)[O-])c1]

To a solution of 3-(methylsulfinylmethyl)-5-nitro-phenol (22 g, 102.22 mmol, 1 eq), 4-[tert-butyl(dimethyl)silyl]oxybutan-1-ol (20.89 g, 102.22 mmol, 1 eq) and PPh₃ (67.03 g, 255.55 mmol, 2.5 eq) in THE (220 mL) was added DIAD (51.67 g, 255.55 mmol, 49.69 mL, 2.5 eq) at 0° C. under N₂. Then the mixture was stirred at 20° C. for 16 hours. TLC showed that the starting material was consumed completely, and one new main spot had formed. LCMS showed that the starting material was consumed completely, and that the desired MS was detected. The reaction mixture was diluted with ethyl acetate (150 mL). The organic layer was washed with Na₂CO₃ (2×150 mL), NH₄C₁ (3×150 mL), H₂O (150 mL) and brine (150 mL), dried over Na₂SO₄, filtered, and concentrated in vacuo to give the crude product. The crude product was purified by column chromatography (SiO₂, petroleum ether:ethyl acetate=3:1 to 1:5) to give B14-1 (62.4 g, 128.97 mmol, 63.0% yield, 83% purity) as a red oil. ¹H-NMR (400 MHz, CDCl₃): δ=7.76-7.71 (m, 2H), 7.19 (s, 1H), 4.12-3.92 (m, 4H), 3.70 (t, J=6.2 Hz, 2H), 2.54 (s, 3H), 1.96-1.85 (m, 2H), 1.75-1.64 (m, 2H), 0.91 (s, 9H), 0.07 (s, 6H).

Step 2: Synthesis of tert-butyl N-[[3-[4-[tert-butyl(dimethyl)silyl]oxybutoxy]-5-nitro-phenyl]methyl-methyl-oxo-λ⁶-sulfanylidene]carbamate (B14-2)

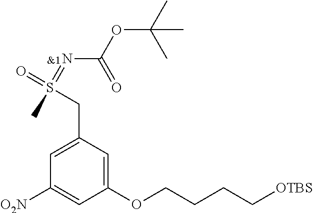 [SMILES: CC(C)(C)OC(=O)N=[S@@](C)(=O)Cc1cc(OCCCCO[Si](C)(C)C(C)(C)C)cc([N+](=O)[O-])c1]

To a solution of B14-1 (20.8 g, 51.79 mmol, 1 eq), tertbutyl carbamate (9.10 g, 77.69 mmol, 1.5 eq), MgO (8.35 g, 207.18 mmol, 2.33 mL, 4 eq) and PhI(OAc)₂ (25.02 g, 77.69 mmol, 1.5 eq) in DCM (200 mL) was added Rh₂(OAc)₄ (572.31 mg, 1.29 mmol, 0.025 eq) at 20° C. under N₂. Then the mixture was stirred at 45° C. for 16 hours. The mixture was filtered and concentrated under reduced pressure to give a residue. The residue was diluted with ethyl acetate (100 mL). The organic layer was washed with NaHCO₃ (100 mL), NH₄C₁ (2×100 mL) and brine (100 mL), dried over Na₂SO₄, filtered, and concentrated under reduced pressure to give a crude product. The crude product was purified by column chromatography (SiO₂, petroleum ether:ethyl acetate=5:1 to 1:1) to give B14-2 (78 g, 125.29 mmol, 80.6% yield, 83% purity) as a yellow gum. ¹H-NMR (400 MHz, CDCl₃): δ=7.84 (s, 1H), 7.77 (t, J=2.0 Hz, 1H), 7.31 (d, J=1.8 Hz, 1H), 4.87-4.76 (m, 2H), 4.09 (t, J=6.4 Hz, 2H), 3.70 (t, J=6.0 Hz, 2H), 3.01 (s, 3H), 1.98-1.83 (m, 2H), 1.76-1.63 (m, 2H), 1.53 (s, 9H), 0.91 (s, 9H), 0.07 (s, 6H).

Step 3: Synthesis of tert-butyl N-[[3-(4-hydroxybutoxy)-5-nitro-phenyl]methyl-methyl-oxo-λ⁶-sulfanylidene]carbamate (B14-3)

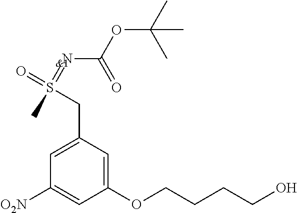 [SMILES: CC(C)(C)OC(=O)N=[S@@](C)(=O)Cc1cc(OCCCCO)cc([N+](=O)[O-])c1]

To a solution of B14-2 (34 g, 65.80 mmol, 1 eq) in THE (340 mL) was added TBAF (1 M, 65.80 mL, 1 eq) at 20° C. under the N₂. Then the mixture was stirred at 20° C. for 1 hour. The reaction mixture was diluted with ethyl acetate (100 mL). The organic layer was washed with NaHCO₃ (100 mL), NH₄C₁ (3×100 mL), H₂O (100 mL) and brine (100 mL), dried over Na₂SO₄, filtered, and concentrated under reduced pressure to give a crude product. The crude product was purified by column chromatography (SiO₂, petroleum ether:ethyl acetate=3:1 to 1:3) to give B14-3 (41.8 g, 97.63 mmol, 70% yield, 94% purity) as a yellow gum. ¹H-NMR (400 MHz, CDCl₃): δ=7.78 (s, 1H), 7.72 (t, J=2.0 Hz, 1H), 6=7.33 (s, 1H), 4.77 (s, 2H), 4.10-4.01 (m, 2H), 3.69 (q, J=6.0 Hz, 2H), 2.96 (s, 3H), 1.93-1.82 (m, 2H), 1.75-1.65 (m, 2H), 1.46 (s, 9H).

Step 4: Synthesis of tert-butyl N-[[3-[4-[2-(2-chloro-5-fluoro-pyrimidin-4-yl)-5-fluoro-phenoxy]butoxy]-5-nitro-phenyl]methyl-methyl-oxo-X⁶-sulfanylidene]carbamate (B14-4)

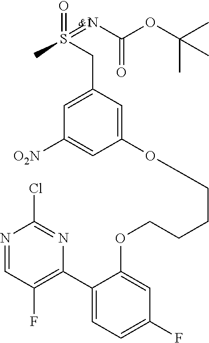 [SMILES: CC(C)(C)OC(=O)N=[S@@](C)(=O)Cc1cc(OCCCCOc2cc(F)ccc2-c2nc(Cl)ncc2F)cc([N+](=O)[O-])c1]

To a solution of B14-3 (10 g, 24.85 mmol, 1 eq) and 2-(2-chloro-5-fluoro-pyrimidin-4-yl)-5-fluoro-phenol (6.03 g, 24.85 mmol, 1 eq) in toluene (100 mL) was added CMBP (11.99 g, 49.69 mmol, 2 eq) at 20° C. under N₂. Then the mixture was stirred at 110° C. for 16 hours. The reaction mixture was diluted with ethyl acetate (50 mL). The organic layer was washed with NH₄C₁ (2×50 mL), H₂O (50 mL) and brine (50 mL), dried over Na₂SO₄, filtered, and concentrated under reduced pressure to give a crude product. The crude product was purified by column chromatography (SiO₂, petroleum ether:ethyl acetate=2:1 to 0:1) to give B14-4 (12.09 g, 17.93 mmol, 72.1% yield, 93% purity) as a yellow foam. ¹H-NMR (400 MHz, CDCl₃): δ=8.58-8.41 (m, 1H), 7.85 (s, 1H), 7.73 (s, 1H), 7.52 (dd, J=6.4, 8.4 Hz, 1H), 7.29 (br d, J=1.6 Hz, 1H), 6.87-6.79 (m, 1H), 6.74 (dd, J=2.2, 10.8 Hz, 1H), 4.83 (s, 2H), 4.18-4.04 (m, 4H), 3.07-3.02 (m, 3H), 2.02-1.92 (m, 4H), 1.56-1.50 (m, 9H).

Step 5: Synthesis of tert-butyl N-[[3-amino-5-[4-[2-(2-chloro-5-fluoro-pyrimidin-4-yl)-5-fluoro-phenoxy]butoxy]phenyl]methyl-methyl-oxo-X⁶-sulfanylidene]carbamate (B14-5)

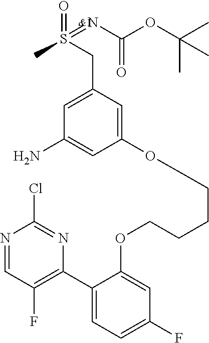 [SMILES: CC(C)(C)OC(=O)N=[S@@](C)(=O)Cc1cc(N)cc(OCCCCOc2cc(F)ccc2-c2nc(Cl)ncc2F)c1]

To a solution of B14-4 (11.5 g, 18.34 mmol, 1 eq) in EtOH (100 mL) and H₂O (20 mL) was added Fe (5.12 g, 91.70 mmol, 5 eq) and NH₄C₁ (4.91 g, 91.70 mmol, 5 eq) at 20° C. under N₂. Then the mixture was stirred at 80° C. for 2 hours. The mixture was filtered through a pad of celite, and the filter cake was washed with ethyl acetate (3×50 mL). The organic layer was concentrated under reduced pressure to remove EtOH. The residue was diluted with ethyl acetate (50 mL). The organic layer was washed with H₂O (50 mL) and brine (50 mL), dried over Na₂SO₄, filtered, and concentrated under reduced pressure to give a crude product. The crude product was purified by column chromatography (SiO₂, petroleum ether:ethyl acetate=2:1 to 1:1) to give B14-5 (13.39 g, 20.86 mmol, 90% yield, 93% purity) as a yellow foam. ¹H-NMR (400 MHz, CDCl₃): δ=8.49-8.42 (m, 1H), 7.56-7.49 (m, 1H), 6.82 (br d, J=2.0 Hz, 1H), 6.76-6.71 (m, 1H), 6.41-6.31 (m, 2H), 6.24-6.20 (m, 1H), 4.61 (s, 2H), 4.17-4.07 (m, 2H), 3.92 (s, 2H), 2.97 (s, 3H), 1.96-1.82 (m, 4H), 1.52 (s, 9H).

Step 6: Synthesis of tert-butyl N-[(5,23-difluoro-8,13-dioxa-19,21,24-triazatetracyclo[18.3.1.114,18.02,7]pentacosa-1(23),2,4,6,14,16,18(25),20(24),21-nonaen-16-yl)methyl-methyl-oxo-X⁶-sulfanylidene]carbamate (B14-6)

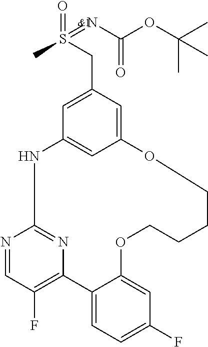 [SMILES: CC(C)(C)OC(=O)N=[S@@](C)(=O)Cc1cc2cc(c1)OCCCCOc1cc(F)ccc1-c1nc(ncc1F)N2]

To a solution of B14-5 (7 g, 11.72 mmol, 1 eq) in toluene (70 mL) and NMP (7 mL) was added K₃PO₄ (12.44 g, 58.62 mmol, 5 eq), XPhos (558.90 mg, 1.17 mmol, 0.1 eq) and XPhos Pd G1 (433.06 mg, 586.19 umol, 0.05 eq). The mixture was stirred at 110° C. for 3 hours under N₂. TLC showed that B14-5 was consumed completely and that many new spots formed. The desired MS was detected by LCMS. The mixture was filtered through a pad of Celite and the filter cake was washed with ethyl acetate (3×50 mL). The organic layer was washed with NaHCO₃ (50 mL), NH₄C₁ (2×50 mL), H₂O (50 mL), brine (50 mL), dried over Na₂SO₄, filtered, and concentrated under reduced pressure to give a crude product. The crude product was purified by column chromatography (SiO₂, petroleum ether:ethyl acetate=3:1 to 1:1) to give B14-6 (3.16 g, 5.58 mmol, 47.6% yield, 99% purity) as a yellow foam. ¹H-NMR (400 MHz, CDCl₃): δ=8.42-8.39 (m, 1H), 8.22 (s, 1H), 7.37-7.29 (m, 1H), 6.83-6.70 (m, 2H), 6.54 (d, J=1.2 Hz, 2H), 4.64 (s, 2H), 4.23 (br d, J=5.8 Hz, 4H), 2.96 (s, 3H), 2.05 (s, 2H), 1.99-1.91 (m, 2H), 1.52 (s, 9H).

Step 7: Synthesis of (5,23-difluoro-8,13-dioxa-19,21,24-triazatetracyclo[18.3.1.114,18.02,7]pentacosa-1(23),2,4,6,14,16,18(25),20(24),21-nonaen-16-yl)methyl-imino-methyl-oxo-λ⁶-sulfane (B14-7)

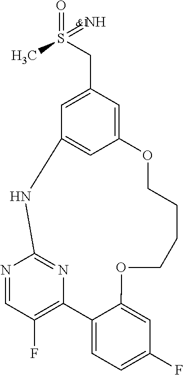 [SMILES: C[S@](=N)(=O)Cc1cc2cc(c1)OCCCCOc1cc(F)ccc1-c1nc(ncc1F)N2]

To a mixture of B14-6 (4 g, 7.14 mmol, 1 eq) in DCM (40 mL) was added TFA (4 mL) in a dropwise fashion. The mixture was stirred at 20° C. for 2 hours. The reaction mixture was concentrated under reduced pressure to give a residue (B14-7), which was used immediately in the next step.

Step 8: Chiral separation of B14-7 to give Building block 14 and building block 15 (two single enantiomers)

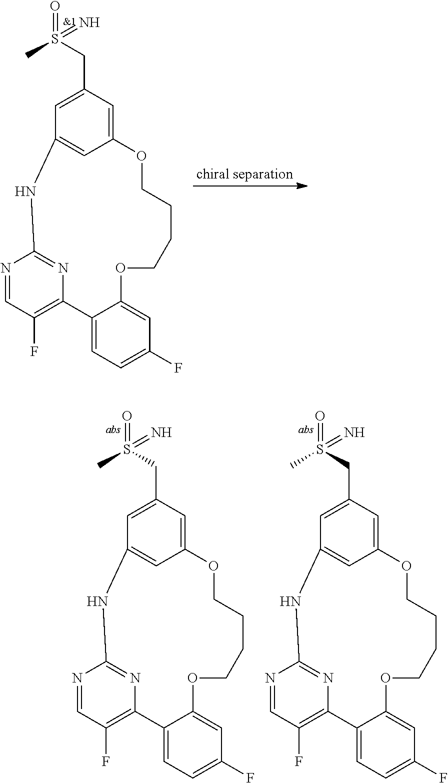

B14-7 was separated by prep-SFC (column: DAICEL CHIRALPAK AD (250 mm*50 mm, 10 um); mobile phase: [ACN/EtOH(0.1% NH₃H₂O)]; B %:60%-60%, 21 min, Rt1=3.552, Rt2=4.801) to give Building block 14 (813.24 mg, 16.8% yield, 99.1% purity) as a light-yellow solid and Building block 15 (690.45 mg, 13.9% yield, 96.5% purity) as an off-white solid. Note: The R/S configuration of these two compounds were not confirmed.

Building block 14: ¹H NMR (400 MHz, DMSO-d₆) δ 9.76 (s, 1H), 8.74-8.59 (m, 1H), 7.95 (s, 1H), 7.39 (ddd, J=3.0, 6.8, 8.6 Hz, 1H), 7.16 (dd, J=2.4, 11.8 Hz, 1H), 6.93-6.83 (m, 1H), 6.68 (s, 1H), 6.51 (s, 1H), 4.26-4.12 (m, 6H), 3.54 (s, 1H), 2.81 (s, 3H), 1.87 (br s, 4H). The desired enantiomer (optical rotation 7.170°±0.00°, 20 C, 589 nm) was obtained with 100% ee at Rₜ 3.60-4.15 min. LC-MS: Rₜ=2.26 min; MS (ESIpos): m/z=461 [M+H]⁺. Single (+) isomer, absolute stereochemistry unknown.

Building block 15: ¹H NMR (400 MHz, DMSO-d₆) δ 9.76 (s, 1H), 8.65 (d, J=1.8 Hz, 1H), 7.95 (s, 1H), 7.46-7.30 (m, 1H), 7.15 (dd, J=1.8, 11.8 Hz, 1H), 6.87 (dt, J=2.0, 8.2 Hz, 1H), 6.74-6.60 (m, 1H), 6.50 (s, 1H), 4.38-4.16 (m, 4H), 4.19-4.03 (m, 2H), 3.68 (br s, 1H), 2.81 (s, 3H), 1.86 (br s, 4H). The desired enantiomer (optical rotation −5.51°±0.28°, 20 C, 589 nm) was obtained with 100% ee at Rₜ 4.60-6.00 min. LC-MS: Rₜ=2.26 min; MS (ESIpos): m/z=461 [M+H]⁺. Single (−) isomer, absolute stereochemistry unknown.

Examples B16, B17, B18 & B19: Preparation of [5,22-difluoro-11-methyl-8,12-dioxa-18,20,23-triazatetracyclo[17.3.1.113,17.02,7]tetracosa-1(22),2,4,6,13,15,17(24),19(23),20-nonaen-15-yl]methyl-imino-methyl oxo-λ⁶-sulfane (four single isomers) (Building block 16, Building block 17, Building block 18, Building block 19)

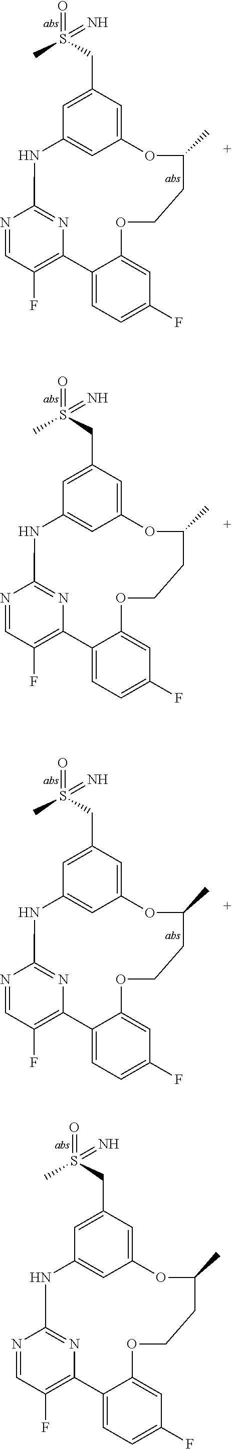 [SMILES: C[C@@H]1CCOc2cc(F)ccc2-c2nc(ncc2F)Nc2cc(C[S@@](C)(=N)=O)cc(c2)O1 ; C[C@@H]1CCOc2cc(F)ccc2-c2nc(ncc2F)Nc2cc(C[S@](C)(=N)=O)cc(c2)O1 ; C[C@H]1CCOc2cc(F)ccc2-c2nc(ncc2F)Nc2cc(C[S@@](C)(=N)=O)cc(c2)O1 ; C[C@H]1CCOc2cc(F)ccc2-c2nc(ncc2F)Nc2cc(C[S@](C)(=N)=O)cc(c2)O1]

Building block 16, 17, 18 and 19 were prepared from commercially available intermediates according to the following steps.

Step 1: Synthesis of 4-[tert-butyl(diphenyl)silyl]oxybutan-2-ol (B16-1)

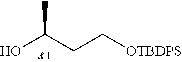

To a solution of butane-1,3-diol (20 g, 221.9 mmol, 1 eq) in DCM (200 mL) was added imidazole (30.22 g, 443.8 mmol, 2 eq) and TBDPSCl (61.0 g, 221.9 mmol, 57.01 mL, 1 eq) at 0° C. The mixture was stirred at 0° C. for 1 hour. The reaction mixture was diluted with DCM (300 mL). The organic layer was washed with water 600 mL (2×300 mL), dried over Na₂SO₄, filtered, and concentrated under reduced pressure to give a residue. The residue was purified by column chromatography (SiO₂, petroleum ether/ethyl acetate=10:1 to 2:1) to give B16-1 (58.2 g, 177.16 mmol, 79.8% yield) as a colorless oil.

¹H NMR (400 MHz, CDCl₃-d) δ 7.73-7.66 (m, 4H), 7.50-7.36 (m, 6H), 4.11 (dt, J=2.8, 6.0 Hz, 1H), 3.88 (s, 2H), 3.28 (d, J=2.6 Hz, 1H), 1.81-1.60 (m, 2H), 1.22 (d, J=6.0 Hz, 3H), 1.06 (s, 9H).

Step 2: Synthesis of tert-butyl-[3-[3-(methylsulfinylmethyl)-5-nitro-phenoxy]butoxy]-diphenyl-silane (B16-2)

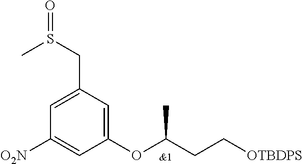

To a solution of 3-(methylsulfinylmethyl)-5-nitro-phenol (28 g, 130.10 mmol, 1 eq), B16-1 (42.74 g, 130.10 mmol, 1 eq) and PPh₃ (85.31 g, 325.24 mmol, 2.5 eq) in THF (500 mL) was added DIAD (65.77 g, 325.24 mmol, 63.24 mL, 2.5 eq) at 0° C. under N₂. The mixture was stirred at 20° C. for 16 hours. The reaction mixture was diluted with EtOAc (500 mL). The organic layer was washed with NaHCO₃ (200 ml), NH₄C₁ (200 mL), H₂O (200 mL) and brine (200 mL), dried over Na₂SO₄, filtered, and concentrated under reduced pressure to give a residue. The residue was purified by column chromatography (SiO₂, petroleum ether/ethyl acetate=5:1 to 0:1) to give B16-2 (62 g, 117.93 mmol, 90.6% yield) as a light-yellow oil. ¹H NMR (400 MHz, CDCl₃-d) δ 7.74-7.53 (m, 6H), 7.50-7.28 (m, 6H), 7.18-7.11 (m, 1H), 4.86-4.73 (m, 1H), 4.01-3.90 (m, 2H), 3.89-3.73 (m, 2H), 2.51 (d, J=5.6 Hz, 3H), 2.07-1.78 (m, 2H), 1.34 (d, J=6.0 Hz, 3H), 1.04 (s, 9H).

Step 3: Synthesis of tert-butyl N-[[3-[3-[tert-butyl(diphenyl)silyl]oxy-1-methyl-propoxy]-5-nitro-phenyl]methyl-methyl-oxo-X⁶-sulfanylidene]carbamate (B16-3)

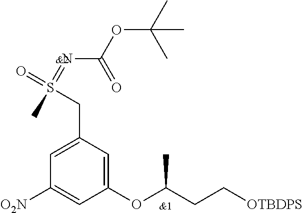

To a mixture of B16-2 (23.5 g, 44.70 mmol, 1 eq), tert-butyl carbamate (7.85 g, 67.05 mmol, 1.5 eq), MgO (7.21 g, 178.80 mmol, 2.01 mL, 4 eq) and PhI(OAc)₂ (21.60 g, 67.05 mmol, 1.5 eq) in DCM (350 mL) was added Rh₂(OAc)₄ (493.92 mg, 1.12 mmol, 0.025 eq) at 20° C. under N₂. Then mixture was stirred at 45° C. for 16 hours. The reaction mixture was filtered and concentrated under reduced pressure to give a residue. The residue was purified by column chromatography (SiO₂, petroleum ether/ethyl acetate=1:1 to 0:1) to give B16-3 (62 g, 91.91 mmol, 68.5% yield, 95% purity) as a light-yellow oil. ¹H NMR (400 MHz, CDCl₃-d) δ 7.82 (d, J=1.4 Hz, 1H), 7.75 (t, J=2.0 Hz, 1H), 7.61 (dd, J=6.6, 17.6 Hz, 4H), 7.48-7.28 (m, 6H), 7.24 (s, 1H), 4.89-4.69 (m, 3H), 3.91-3.73 (m, 2H), 2.97 (s, 3H), 2.04-1.82 (m, 2H), 1.52 (s, 9H), 1.34 (d, J=6.0 Hz, 3H), 1.04 (s, 9H).

Step 4: Synthesis of tert-butyl N-[[3-(3-hydroxy-1-methyl-propoxy)-5-nitro-phenyl]methyl-methyl-oxo-λ⁶-sulfanylidene]carbamate (B16-4)

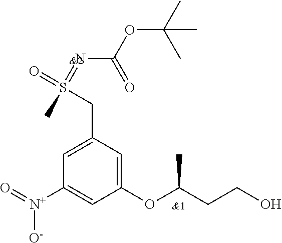 [SMILES: C[C@@H](CCO)Oc1cc(C[S@](C)(=O)=NC(=O)OC(C)(C)C)cc([N+](=O)[O-])c1]

Two reactions were carried out in parallel. To a solution of B16-3 (30 g, 46.81 mmol, 1 eq) in THE (40 mL) was added TBAF (1 M, 46.81 mL, 1 eq) at 20° C. The mixture was stirred at 20° C. for 1 hour. The reaction mixture was concentrated under reduced pressure to give a residue. The two reactions mixture were combined and diluted with EtOAc (200 mL). The organic layer was washed with NH₄C₁ (50 mL), NaHCO₃ (50 mL), dried over Na₂SO₄, filtered, and concentrated under reduced pressure to give a residue. The residue was purified by column chromatography (SiO₂, petroleum ether/ethyl acetate=1:1 to 0:1) to give B16-4 (32 g, 79.51 mmol, 84.9% yield) as a light-yellow oil. ¹H NMR (400 MHz, CDCl₃) δ 7.83-7.79 (m, 2H), 7.42 (d, J=1.6 Hz, 1H), 4.82-4.72 (m, 3H), 3.84-3.72 (m, 2H), 3.07 (d, J=4.4 Hz, 3H), 2.04-1.96 (m, 1H), 1.93-1.81 (m, 1H), 1.51 (s, 9H), 1.39 (dd, J=1.6, 6.0 Hz, 3H).

Step 5: Synthesis of tert-butyl N-[[3-[3-[2-(2-chloro-5-fluoro-pyrimidin-4-yl)-5-fluoro-phenoxy]-1-methyl-propoxy]-5-nitro-phenyl]methyl-methyl-oxo-λ⁶-sulfanylidene]carbamate

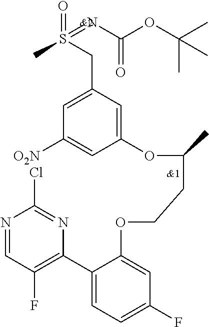 [SMILES: C[C@@H](CCOc1cc(F)ccc1-c1nc(Cl)ncc1F)Oc1cc(C[S@](C)(=O)=NC(=O)OC(C)(C)C)cc([N+](=O)[O-])c1]

To a solution of B16-4 (18 g, 44.72 mmol, 1 eq) and 2-(2-chloro-5-fluoro-pyrimidin-4-yl)-5-fluoro-phenol (10.85 g, 44.72 mmol, 1 eq) in toluene (180 mL) was added 2-(tributyl-λ⁵-phosphanylidene)acetonitrile (16.19 g, 67.09 mmol, 1.5 eq) at 20° C. under N₂. The mixture was stirred at 80° C. for 16 hours. The reaction mixture was diluted with EtOAc (150 mL). The organic layer was washed with H₂O (3×100 mL), NaHCO₃ (3×100 mL), brine (50 mL), dried over Na₂SO₄, filtered, and concentrated under reduced pressure to give a residue. The residue was purified by column chromatography (SiO₂, petroleum ether/ethyl acetate=3:1 to 1:1) to give B16-5 (24 g, 38.27 mmol, 85.5% yield) as a light-yellow oil. ¹H NMR (400 MHz, CDCl₃) δ 8.55 (dd, J=1.5, 3.6 Hz, 1H), 7.85-7.80 (m, 1H), 7.76 (t, J=2.0 Hz, 1H), 7.51 (dd, J=6.5, 8.5 Hz, 1H), 7.34-7.28 (m, 1H), 6.81 (dt, J=2.3, 8.3 Hz, 1H), 6.72 (dd, J=2.0, 10.5 Hz, 1H), 4.84-4.70 (m, 3H), 4.23-4.15 (m, 2H), 2.99 (d, J=4.9 Hz, 3H), 2.19-2.07 (m, 2H), 1.54-1.50 (m, 9H), 1.42-1.35 (m, 3H).

Step 6: Synthesis of tert-butyl N-[[3-amino-5-[3-[2-(2-chloro-5-fluoro-pyrimidin-4-yl)-5-fluoro-phenoxy]-1-methyl-propoxy]phenyl]methyl-methyl-oxo-λ⁶-sulfanylidene]carbamate

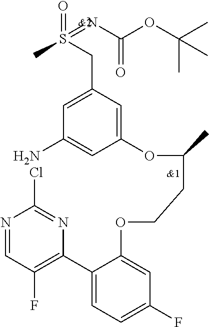 [SMILES: C[C@@H](CCOc1cc(F)ccc1-c1nc(Cl)ncc1F)Oc1cc(N)cc(C[S@](C)(=O)=NC(=O)OC(C)(C)C)c1]

To a solution of B16-5 (22 g, 35.08 mmol, 1 eq) in EtOH (50 mL) and H₂O (10 mL) was added Fe (9.80 g, 175.42 mmol, 5 eq) and NH₄C₁ (9.38 g, 175.42 mmol, 5 eq) at 20° C. The mixture was stirred at 80° C. for 2 hours. The reaction mixture was filtered and concentrated under reduced pressure to give a residue. The residue was diluted with EtOAc (200 mL). The organic layer was washed with H₂O (2×50 mL), brine (50 mL), dried over Na₂SO₄, filtered, and concentrated under reduced pressure to give a residue. The residue was purified by column chromatography (SiO₂, petroleum ether/ethyl acetate=2:1 to 1:1) to give B16-6 (18.5 g, 30.98 mmol, 88.3% yield) as a light-yellow oil. ¹H NMR (400 MHz, CDCl₃) δ 8.50 (dd, J=1.4, 4.6 Hz, 1H), 7.49 (dd, J=6.6, 8.6 Hz, 1H), 6.86-6.67 (m, 2H), 6.39-6.22 (m, 2H), 6.16 (s, 1H), 4.67-4.39 (m, 3H), 4.23-4.04 (m, 2H), 2.91 (d, J=1.4 Hz, 3H), 2.15-1.96 (m, 2H), 1.56-1.47 (m, 9H), 1.28 (d, J=5.8 Hz, 3H).

Step 7: Synthesis of tert-butyl N-[(5,22-difluoro-11-methyl-8,12-dioxa-18,20,23-triazatetracyclo[17.3.1.113,17.02,7]tetracosa-1(22),2,4,6,13,15,17(24),19(23),20-nonaen-15-yl)methyl-methyl-oxo-λ⁶-sulfanylidene]carbamate (B16-7)

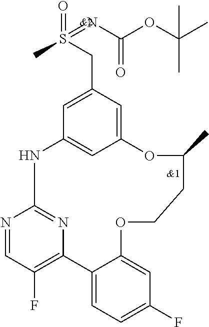 [SMILES: C[C@H]1CCOc2cc(F)ccc2-c2nc(ncc2F)Nc2cc(C[S@](C)(=O)=NC(=O)OC(C)(C)C)cc(c2)O1]

To a solution of B16-6 (13 g, 21.77 mmol, 1 eq) in toluene (90 mL) and NMP (9 mL) were added K₃PO₄ (23.11 g, 108.86 mmol, 5 eq), XPhos (1.04 g, 2.18 mmol, 0.1 eq) and Xhos Pd G1 (1.61 g, 2.18 mmol, 0.1 eq) at 20° C. under N₂. The mixture was stirred at 110° C. for 5 hours. The reaction mixture was diluted with EtOAc (200 mL). The organic layer was washed with H₂O (2×50 mL), dried over Na₂SO₄, filtered, and concentrated under reduced pressure to give a residue. The residue was purified by prep-TLC (SiO₂, petroleum ether/ethyl acetate=1:1.5) to give B16-7 (8.2 g, 14.33 mmol, 65.8% yield, 98% purity) as a light-yellow gum. ¹H NMR (400 MHz, CDCl₃) δ 8.81 (d, J=2.0 Hz, 1H), 8.39 (d, J=3.0 Hz, 1H), 7.67-7.59 (m, 1H), 7.30 (br s, 1H), 6.92-6.72 (m, 2H), 6.59-6.46 (m, 2H), 4.78-4.52 (m, 3H), 4.25-4.14 (m, 2H), 2.98 (d, J=3.8 Hz, 3H), 2.57 (br t, J=12.8 Hz, 1H), 1.86-1.76 (m, 1H), 1.54 (d, J=3.4 Hz, 9H), 1.47 (d, J=6.0 Hz, 3H).

Step 8: Synthesis of (5,22-difluoro-11-methyl-8,12-dioxa-18,20,23-triazatetracyclo[17.3.1.113,17.02,7]tetracosa-1(22),2,4,6,13,15,17(24),19(23),20-nonaen-15-yl)methyl-imino-methyl-oxo-λ⁶-sulfane (B16-8)

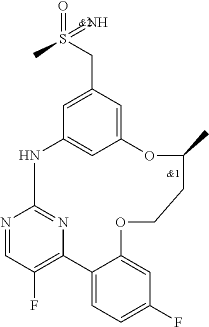 [SMILES: C[C@H]1CCOc2cc(F)ccc2-c2nc(ncc2F)Nc2cc(C[S@@](C)(=N)=O)cc(c2)O1]

To a solution of B16-7 (6 g, 10.70 mmol, 1 eq) in DCM (60 mL) was added TFA (6 mL) at 20° C. The mixture was stirred at 20° C. for 1 hour. The reaction mixture was concentrated under reduced pressure to give crude product B16-8, which was used in the next step.

Step 9: Chiral Separation of B16-8 to Give Building Block 16, Building Block 17, Building Block 18 and Building Block 19

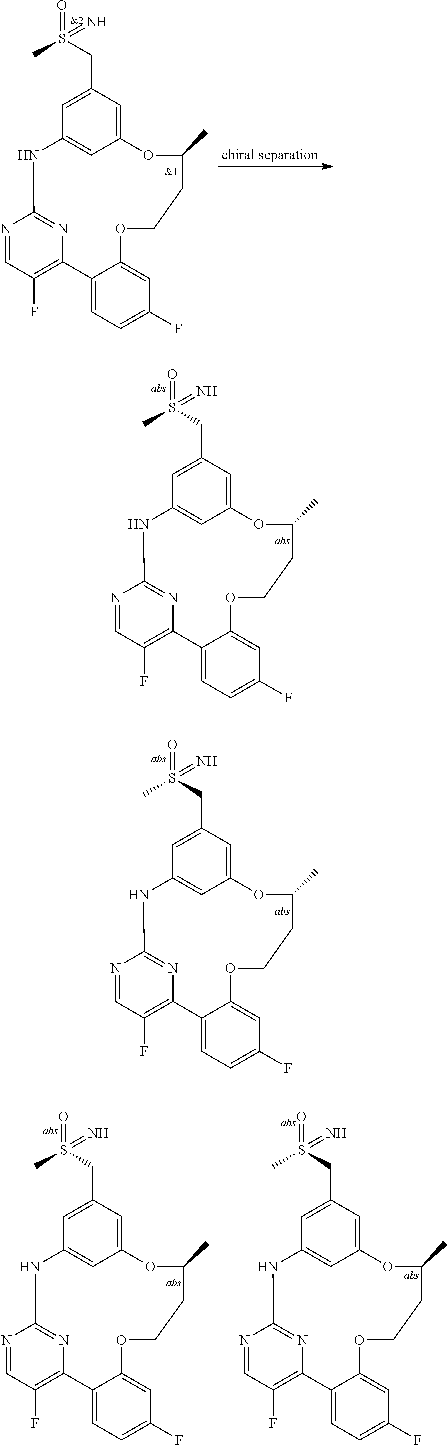

B16-8 was separated by prep-SFC (column: DAICEL CHIRALPAK AS (250 mm*50 mm 10 um); mobile phase: [0.1% NH₃H₂O ETOH]; B %: 60%-60%, 8.4 min) to give P1 (2 isomers), P2 (Building block 16), P3 (Building block 17). Then, the P1 residue was repurified by prep-SFC (column: DAICEL CHIRALPAK AD (250 mm*30 mm, 10 um); mobile phase: [0.1% NH₃H₂O ETOH]; B %: 55%-55%, 7 min) to give P4 (Building block 18) and P5 (Building block 19). P2 (Building block 16) (1.1 g, 2.34 mmol, 21.8% yield, 98% purity) was obtained as a light-yellow solid. P3 (Building block 17) (1 g, 2.13 mmol, 19.8% yield, 98% purity) was obtained as a light yellow solid. P4 (Building block 18) (1.2 g, 2.61 mmol, 24.3% yield) and P5 (Building block 19) (1.3 g, 2.82 mmol, 26.3% yield) was obtained as a light-yellow solid.

Note: The R/S configuration of these four compounds were not confirmed.

Building block 16: ¹H NMR (400 MHz, DMSO-d₆) δ=9.80 (s, 1H), 8.67 (d, J=2.7 Hz, 2H), 7.66-7.57 (m, 1H), 7.35 (dd, J=2.1, 12.1 Hz, 1H), 6.92 (dt, J=2.1, 8.3 Hz, 1H), 6.74 (s, 1H), 6.47 (s, 1H), 4.47 (br t, J=11.0 Hz, 1H), 4.37 (br dd, J=6.1, 9.8 Hz, 1H), 4.22 (q, J=13.4 Hz, 2H), 4.08 (br d, J=10.5 Hz, 1H), 3.55 (s, 1H), 2.82 (s, 3H), 2.48-2.27 (m, 1H), 1.77-1.64 (m, 1H), 1.43 (d, J=6.1 Hz, 3H). The desired enantiomer (optical rotation −112.52°±0.00°, 20 C, 589 nm) was obtained with 96.52% ee at Rₜ 1.45-1.64 min. LC-MS: Rₜ=2.31 min; MS (ESIpos): m/z=461 [M+H]⁺. Single (−) isomer, absolute stereochemistry unknown.

Building block 17: ¹H NMR (400 MHz, DMSO-d₆) δ=9.83 (s, 1H), 8.71-8.66 (m, 2H), 7.62 (ddd, J=4.5, 7.1, 8.5 Hz, 1H), 7.36 (dd, J=2.2, 12.1 Hz, 1H), 6.92 (dt, J=2.2, 8.3 Hz, 1H), 6.75 (s, 1H), 6.49 (s, 1H), 4.47 (br t, J=10.9 Hz, 1H), 4.41-4.31 (m, 3H), 4.13-4.04 (m, 1H), 2.95 (s, 3H), 2.52-2.52 (m, 1H), 2.41-2.29 (m, 2H), 1.77-1.66 (m, 1H), 1.44 (d, J=6.0 Hz, 3H). The desired enantiomer (optical rotation −129.68°±0.70°, 20 C, 589 nm) was obtained with 98.04% ee at Rₜ 1.60-1.83 min. LC-MS: Rₜ=2.32 min; MS (ESIpos): m/z=461 [M+H]⁺. Single (−) isomer, absolute stereochemistry unknown.

Building block 18: ¹H NMR (400 MHz, DMSO-d₆) δ 9.81 (s, 1H), 8.67 (d, J=3.0 Hz, 2H), 7.62 (ddd, J=4.6, 7.0, 8.4 Hz, 1H), 7.35 (dd, J=2.0, 12.0 Hz, 1H), 6.92 (dt, J=2.2, 8.2 Hz, 1H), 6.73 (s, 1H), 6.47 (s, 1H), 4.47 (br t, J=10.8 Hz, 1H), 4.40-4.28 (m, 1H), 4.25-4.17 (m, 2H), 4.08 (br d, J=10.4 Hz, 1H), 3.56 (s, 1H), 2.81 (s, 3H), 2.41-2.27 (m, 1H), 1.81-1.63 (m, 1H), 1.43 (d, J=6.0 Hz, 3H). The desired enantiomer (optical rotation 133.00°±0.00°, 20 C, 589 nm) was obtained with 98.70% ee at Rₜ 2.10-2.80 min. LC-MS: Rₜ=2.33 min; MS (ESIpos): m/z=461 [M+H]⁺. Single (+) isomer, absolute stereochemistry unknown.

Building block 19: ¹H NMR (400 MHz, DMSO-d₆) δ 9.82 (s, 1H), 8.67 (d, J=2.8 Hz, 2H), 7.62 (ddd, J=4.6, 7.0, 8.4 Hz, 1H), 7.36 (dd, J=2.0, 12.0 Hz, 1H), 6.92 (dt, J=2.2, 8.2 Hz, 1H), 6.73 (s, 1H), 6.50-6.38 (m, 1H), 4.47 (br t, J=11.0 Hz, 1H), 4.41-4.32 (m, 1H), 4.28-4.15 (m, 2H), 4.15-4.02 (m, 1H), 3.56 (s, 1H), 2.82 (s, 3H), 2.35 (br t, J=12.6 Hz, 1H), 1.78-1.63 (m, 1H), 1.43 (d, J=6.0 Hz, 3H). The desired enantiomer (optical rotation 120.800±0.00°, 20 C, 589 nm) was obtained with 94.14% ee at Rₜ 2.50-3.05 min. LC-MS: Rₜ=2.33 min; MS (ESIpos): m/z=461 [M+H]⁺. Single (+) isomer, absolute stereochemistry unknown.

Example B20 & Example B21: Preparation of (5,25-difluoro-8,15-dioxa-21,23,27-triazatetracyclo[20.3.1.116,20.02,7]heptacosa-1(25),2,4,6,16,18,20(27),22(26),23-nonaen-18-yl)methyl-imino-methyl-oxo-λ⁶-sulfane (two single enantiomers) (Building block 20, Building block 21)

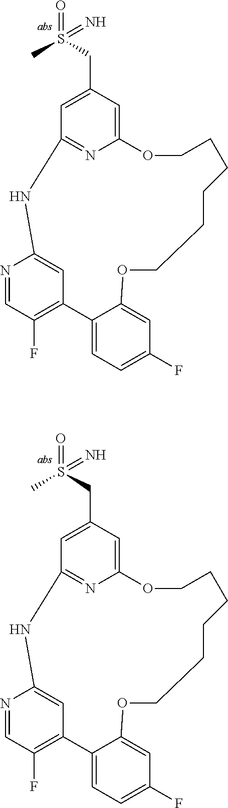 [SMILES: C[S@](=N)(=O)Cc1cc2nc(c1)OCCCCCCOc1cc(F)ccc1-c1cc(ncc1F)N2 ; C[S@@](=N)(=O)Cc1cc2nc(c1)OCCCCCCOc1cc(F)ccc1-c1cc(ncc1F)N2]

Building block 20 and building block 21 were prepared from commercially available intermediates according to the following steps.

Step 1: Synthesis of (2,6-dichloro-4-pyridyl)methyl methanesulfonate (B20-1)

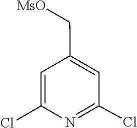 [SMILES: CS(=O)(=O)OCc1cc(Cl)nc(Cl)c1]

To a solution of (2,6-dichloro-4-pyridyl)methanol (43 g, 241.55 mmol, 1 eq) in DCM (645 mL) was added TEA (42.77 g, 422.72 mmol, 58.84 mL, 1.75 eq). The mixture was cooled to 0° C. and MsCl (38.74 g, 338.17 mmol, 26.17 mL, 1.4 eq) was added. The mixture was stirred at 20° C. for 1 hour. LCMS showed that the starting material was consumed, and that the desired MS was detected. The reaction mixture was washed with H₂O (300 mL), NH₄C₁ (200 ml), brine (200 ml), dried over Na₂SO₄, filtered, and concentrated under reduced pressure to give B20-1 (63.3 g, 241.48 mmol, 99.9% yield, 97.7% purity) as a yellow solid. ¹H NMR (400 MHz, CDCl₃-d) δ 7.29-7.24 (m, 2H), 5.20 (s, 2H), 3.11 (s, 3H).

Step 2: Synthesis of 2,6-dichloro-4-(methylsulfanylmethyl)pyridine (B20-2)

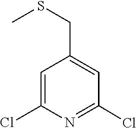

To a solution of B20-1 (63 g, 238.61 mmol, 97% purity, 1 eq) in dioxane (441 mL) and H₂O (63 mL) was added NaSMe (100.34 g, 286.33 mmol, 91.22 mL, 20% purity, 1.2 eq) at 0° C. under N₂. The mixture was stirred at 0° C. for 3 hours. LCMS showed that the starting material was consumed, and the desired MS was detected. The residue was poured into water (300 mL). The aqueous phase was extracted with ethyl acetate (3×100 mL). The combined organic phase was washed with brine (3×100 mL), dried with anhydrous Na₂SO₄, filtered, and concentrated in vacuo. The crude product was purified by column chromatography (SiO₂, petroleum ether:ethyl acetate=0:1 to 24:1) to give B20-2 (99.8 g, crude) as a yellow oil.

¹H NMR (400 MHz, CDCl₃-d) δ 7.23 (s, 2H), 3.59 (s, 2H), 2.03 (s, 3H)

Step 3: Synthesis of 6-[[6-chloro-4-(methylsulfanylmethyl)-2-pyridyl]oxy]hexan-1-ol (B20-3)

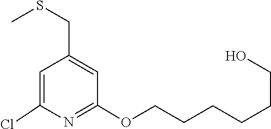 [SMILES: CSCc1cc(Cl)nc(OCCCCCCO)c1]

To a suspension of NaH (2.31 g, 57.66 mmol, 60% purity, 1.2 eq) in THF (100 mL) was added hexane-1,6-diol (14.20 g, 120.13 mmol, 14.34 mL, 2.5 eq) at 0° C. under N₂. The mixture was stirred at 20° C. for 30 min, then B20-2 (10 g, 48.05 mmol, 1 eq) was added to the reaction mixture at 20° C. under N₂. The mixture was stirred at 70° C. for 15.5 h. The reaction mixture was poured into NH₄C₁ (50 mL). The aqueous phase was extracted with ethyl acetate (2×50 mL). The combined organic phase was washed with brine (2×50 mL), dried with anhydrous Na₂SO₄, filtered, and concentrated in vacuo. The crude product was purified by column chromatography (SiO₂, petroleum ether:ethyl acetate=15:1 to 4:1) to give B20-3 (10.8 g, 37.26 mmol, 77.5% yield) as a yellow oil.

¹H NMR (400 MHz, CDCl₃-d) δ 6.87 (s, 1H), 6.57 (s, 1H), 4.28 (t, J=6.6 Hz, 2H), 3.66 (t, J=6.6 Hz, 2H), 3.54 (s, 2H), 2.02 (s, 3H), 1.81-1.72 (m, 2H), 1.66-1.55 (m, 2H), 1.53-1.36 (m, 4H).

Step 3: Synthesis of 4-[2-[6-[[6-chloro-4-(methylsulfanylmethyl)-2-pyridyl]oxy]hexoxy]-4-fluoro-phenyl]-5-fluoro-pyridin-2-amine (B20-4)

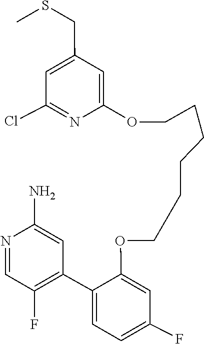 [SMILES: CSCc1cc(Cl)nc(OCCCCCCOc2cc(F)ccc2-c2cc(N)ncc2F)c1]

To a mixture of 2-(2-amino-5-fluoro-4-pyridyl)-5-fluoro-phenol (5.30 g, 23.84 mmol, eq) and B20-3 (7.6 g, 26.22 mmol, 1.1 eq) in THE (70 mL) was added PPh₃ (18.76 g, 71.52 mmol, 3 eq) and DIAD (14.46 g, 71.52 mmol, 13.91 mL, 3 eq) at 0° C. under N₂. The mixture was stirred at 20° C. for 2 hours. The reaction mixture was poured into H₂O (50 mL). The aqueous phase was extracted with ethyl acetate (2×50 mL). The combined organic phase was washed with brine (2×20 mL), dried with anhydrous Na₂SO₄, filtered, and concentrated in vacuo. The residue was purified by column chromatography (SiO₂, petroleum ether:ethyl acetate=16:1 to 2:1) to give B20-4 (10.3 g, crude) as a yellow oil.

¹H NMR (400 MHz, CDCl₃-d) δ 7.95 (s, 1H), 7.24-7.15 (m, 2H), 6.88 (s, 1H), 6.57 (s, 1H), 6.47 (d, J=4.8 Hz, 1H), 6.45-6.36 (m, 1H), 4.41 (s, 2H), 4.26 (t, J=6.6 Hz, 2H), 3.97 (t, J=6.2 Hz, 2H), 3.54 (s, 2H), 2.01 (s, 3H), 1.79-1.70 (m, 4H), 1.44 (td, J=3.6, 7.0 Hz, 4H)

Step 4: Synthesis of 5,25-difluoro-18-(methylsulfanylmethyl)-8,15-dioxa-21,23,27-triazatetracyclo[20.3.1.1\hat{ }{16,20}0.\hat{ }{2,7}]heptacosa-1(25),2,4,6,16,18,20(27),22(26),23-nonaene (B20-5)

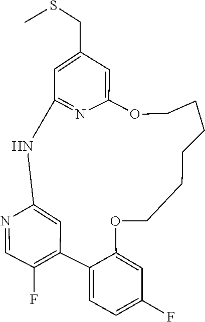 [SMILES: CSCc1cc2nc(c1)OCCCCCCOc1cc(F)ccc1-c1cc(ncc1F)N2]

To a mixture of B20-4 (2.45 g, 4.96 mmol, 1 eq) in toluene (24.5 mL) and NMP (2.45 mL) was added K₃PO₄ (5.26 g, 24.80 mmol, 5 eq), XPhos (236.43 mg, 495.96 umol, 0.1 eq), [2-(2-aminoethyl)phenyl]-chloro-palladium; dicyclohexyl-[2-(2,4,6-triisopropylphenyl)phenyl]phosphane (366.39 mg, 495.96 umol, 0.1 eq) at 20° C. under N₂. The mixture was stirred at 110° C. for 3 hours. The mixture was poured into water (30 mL). The aqueous phase was extracted with ethyl acetate (2×20 mL). The combined organic phase was washed with brine (2×20 mL), dried with anhydrous Na₂SO₄, filtered, and concentrated in vacuo. The residue was purified by column chromatography (SiO₂, petroleum ether:ethyl acetate=24:1 to 3:1) to give B20-5 (2.24 g, crude) as a yellow solid.

¹H NMR (400 MHz, CDCl₃-d) δ 8.18 (d, J=5.6 Hz, 1H), 8.16-8.10 (m, 1H), 7.23-7.15 (m, 2H), 6.77-6.71 (m, 2H), 6.22 (s, 1H), 6.18 (s, 1H), 4.26 (t, J=7.6 Hz, 2H), 4.15 (t, J=5.2 Hz, 2H), 3.52 (s, 2H), 2.03 (s, 3H), 1.76-1.60 (m, 4H), 1.48-1.37 (m, 2H), 1.31-1.21 (m, 2H)

Step 5: Synthesis of (5,25-difluoro-8,15-dioxa-21,23,27-triazatetracyclo[20.3.1.116,20.02,7]heptacosa-1(25),2,4,6,16,18,20(27),22(26),23-nonaen-18-yl)methyl-imino-methyl-oxo-λ⁶-sulfane (B20-6)

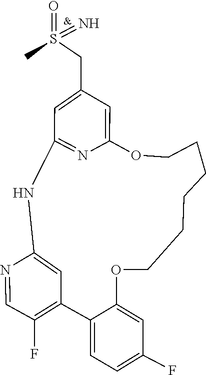 [SMILES: C[S@](=N)(=O)Cc1cc2nc(c1)OCCCCCCOc1cc(F)ccc1-c1cc(ncc1F)N2]

To a mixture of B20-5 (500 mg, 1.09 mmol, 1 eq) in i-PrOH (5 mL) was added PhI(OAc)₂ (1.06 g, 3.28 mmol, 3 eq) and ammonia:carbamic acid (511.90 mg, 6.56 mmol, 6 eq) at 20° C. under N₂. The mixture was stirred at 20° C. for 16 hours. Six reactions were conducted in parallel. The reaction mixtures were combined, diluted with water (50 mL) and extracted with DCM (6×60 mL). The combined organic layers were washed with brine (2×50 mL), dried over Na₂SO₄, filtered, and concentrated under reduced pressure. The residue was purified by column chromatography (SiO₂, petroleum ether/ethyl acetate=1:1 to ethyl acetate:MeOH=50:1) to give B20-6 (1.95 g, 3.99 mmol, 60.87% yield) as a yellow solid.

¹H NMR (400 MHz, CD₃OD-d₄) δ 8.10 (dd, J=1.6, 3.4 Hz, 2H), 7.20 (dd, J=6.8, 8.2 Hz, 1H), 6.94 (dd, J=2.4, 11.2 Hz, 1H), 6.76 (dt, J=2.4, 8.4 Hz, 1H), 6.49 (d, J=1.0 Hz, 1H), 6.27 (d, J=1.0 Hz, 1H), 4.36 (s, 2H), 4.28-4.15 (m, 4H), 2.97 (s, 3H), 1.74-1.55 (m, 4H), 1.45-1.32 (m, 2H), 1.28-1.17 (m, 2H)

Step 6: Chiral Separation of B20-6 to Give Building Block 20 and Building Block 21

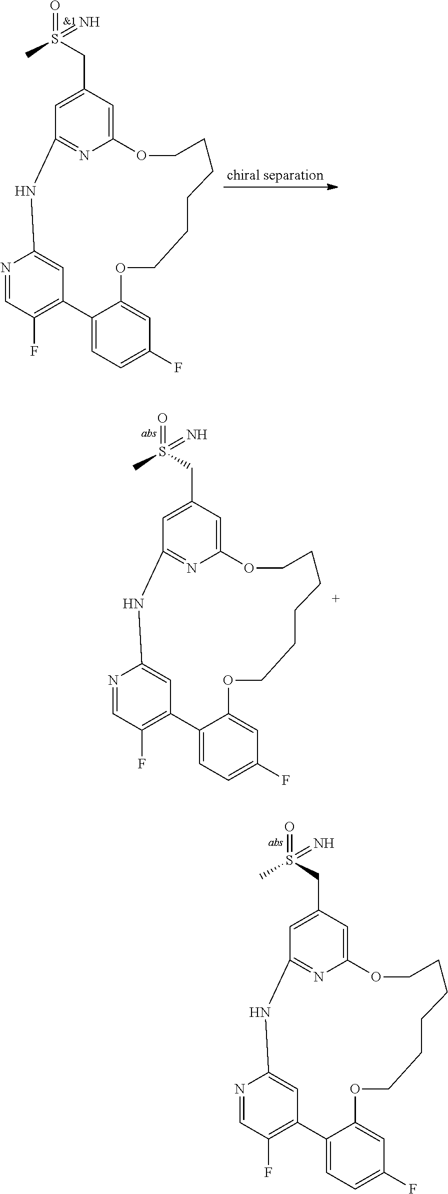

B20-6 was separated by prep-SFC (column: DAICEL CHIRALPAK AD (250 mm*30 mm, 10 um); mobile phase: [0.1% NH₃H₂O ETOH]; B %: 70%-70%, 20 min, Rₜ1=2.242, Rₜ2=2.854) to give Building block 20 (651.31 mg, 1.33 mmol, 33.40% yield) and Building block 21 (742.96 mg, 1.52 mmol, 38.10% yield) as a yellow solid. Note: The R/S configuration of these 2 compounds were not confirmed.

B20: ¹H NMR (400 MHz, CD₃OD-d₄) δ 8.10 (dd, J=1.8, 3.4 Hz, 2H), 7.20 (dd, J=6.8, 8.2 Hz, 1H), 6.94 (dd, J=2.4, 11.2 Hz, 1H), 6.76 (d, J=2.4 Hz, 1H), 6.49-6.47 (m, 1H), 6.27 (s, 1H), 4.35 (s, 2H), 4.25-4.15 (m, 4H), 2.97 (s, 3H), 1.74-1.56 (m, 4H), 1.44-1.30 (m, 2H), 1.30-1.18 (m, 2H). The desired enantiomer (optical rotation 5.580°±0.27°, 20 C, 589 nm) was obtained with 100% ee at Rₜ 2.51-3.60 min. LC-MS: Rₜ=2.53 min; MS (ESIpos): m/z=489 [M+H]⁺. Single (+) isomer, absolute stereochemistry unknown.

B21: ¹H NMR (400 MHz, CD₃OD-d₄) δ 8.10 (dd, J=1.8, 3.4 Hz, 2H), 7.20 (dd, J=6.8, 8.2 Hz, 1H), 6.94 (dd, J=2.4, 11.2 Hz, 1H), 6.76 (dt, J=2.4, 8.4 Hz, 1H), 6.50-6.47 (m, 1H), 6.28-6.26 (m, 1H), 4.36 (s, 2H), 4.29-4.15 (m, 4H), 2.97 (s, 3H), 1.74-1.56 (m, 4H), 1.43-1.33 (m, 2H), 1.30-1.20 (m, 2H). The desired enantiomer (optical rotation −1.57°±0.00°, 20 C, 589 nm) was obtained with 95.98% ee at Rₜ 2.52-3.25 min. LC-MS: Rₜ=2.53 min; MS (ESIpos): m/z=489 [M+H]⁺. Single (−) isomer, absolute stereochemistry unknown.

Example B22 & Example B23: Preparation of (5,22-difluoro-8,12-dioxa-18,20,24-triazatetracyclo[17.3.1.113,17.02,7]tetracosa-1(22),2,4,6,15,17(24),19(23),20-nonaen-15-yl)methyl-imino-methyl-oxo-λ⁶-sulfane (two single enantiomers) (Building block 22, Building block 23)

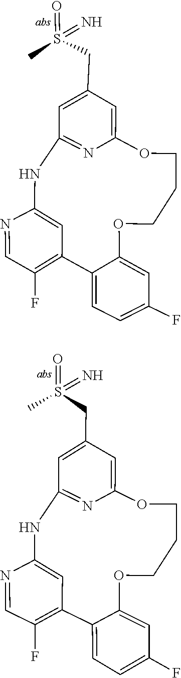

Building block 22 and building block 23 were prepared from commercially available intermediates according to the following steps.

Step 1: Synthesis of 3-[[6-chloro-4-(methylsulfanylmethyl)-2-pyridyl]oxy]propan-1-ol (B22-1)

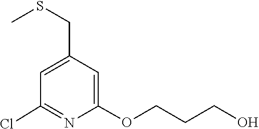

To a solution of propane-1,3-diol (10.97 g, 144.16 mmol, 10.45 mL, 2.5 eq) in THE (144 mL) was added NaH (3.00 g, 74.96 mmol, 60% purity, 1.3 eq) at 0° C. under N₂. The mixture was stirred at 20° C. for 30 min, then 2,6-dichloro-4-(methylsulfanylmethyl)pyridine (12 g, 57.66 mmol, 1 eq) was added to the reaction mixture at 20° C. under N₂. The mixture was stirred at 70° C. for 15.5 h. The reaction mixture was poured into NH₄C₁ (100 mL). The aqueous phase was extracted with ethyl acetate (3×200 mL). The combined organic phase was washed with brine (2×200 mL), dried with anhydrous Na₂SO₄, filtered, and concentrated in vacuo. The crude product was purified by column chromatography (SiO₂, petroleum ether/ethyl acetate=26:1 to 10:1) to give B22-1 (11 g, 44.40 mmol, 77% yield) as a colorless oil. ¹H NMR (400 MHz, CDCl₃) δ 6.90 (s, 1H), 6.59 (d, J=1.0 Hz, 1H), 4.48 (t, J=6.0 Hz, 2H), 3.75 (t, J=5.8 Hz, 2H), 3.54 (s, 2H), 2.45 (br s, 1H), 2.03-1.95 (m, 5H).

Step 2: Synthesis of 4-[2-[3-[[6-chloro-4-(methylsulfanylmethyl)-2-pyridyl]oxy]propoxy]-4-fluoro-phenyl]-5-fluoro-pyridin-2-amine (B22-2)

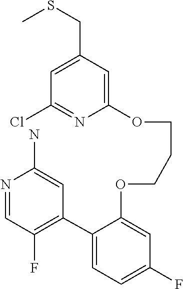 [SMILES: CSCc1cc(Cl)nc(OCCCOc2cc(F)ccc2-c2cc(N)ncc2F)c1]

To a mixture of 3-[[6-chloro-4-(methylsulfanylmethyl)-2-pyridyl]oxy]propan-1-ol (10.43 g, 42.08 mmol, 1.1 eq) and 2-(2-amino-5-fluoro-4-pyridyl)-5-fluoro-phenol (8.5 g, 38.26 mmol, 1 eq) in toluene (100 mL) was added CMBP (27.70 g, 114.77 mmol, 3 eq) at 20° C. under N₂. The mixture was stirred at 110° C. for 16 hours. The reaction mixture was poured into water (40 mL). The aqueous phase was extracted with ethyl acetate (3×50 mL). The combined organic phase was washed with brine (2×50 mL), dried with anhydrous Na₂SO₄, filtered, and concentrated in vacuo. The crude product was purified by column chromatography (SiO₂, petroleum ether/ethyl acetate=20:1 to 8:1) to give B22-2 (16.7 g, 25.87 mmol, 68% yield, 70% purity) as a brown oil. ¹H NMR (400 MHz, CDCl₃-d) δ 7.94 (d, J=2.0 Hz, 1H), 7.21 (dd, J=6.6, 8.8 Hz, 1H), 6.88 (s, 1H), 6.77-6.64 (m, 2H), 6.55 (s, 1H), 6.52-6.43 (m, 1H), 4.46 (s, 2H), 4.42-4.30 (m, 2H), 4.16-4.06 (m, 2H), 3.52 (s, 2H), 2.16 (quin, J=6.0 Hz, 2H), 1.98 (s, 3H).

Step 3: Synthesis of 5,22-difluoro-15-(methylsulfanylmethyl)-8,12-dioxa-18,20,24-triazatetracyclo[17.3.1.1{13,17}.0{2,7}]tetracosa-1(22),2,4,6,13,15,17(24),19(23),20-nonaene

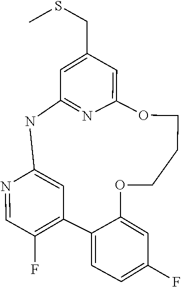

To a mixture of B22-2 (10 g, 15.49 mmol, 70% purity, 1 eq) in toluene (100 mL) and NMP (20 mL) was added K₃PO₄ (16.44 g, 77.45 mmol, 5 eq), XPhos (738.41 mg, 1.55 mmol, 0.1 eq) and Xphos Pd G1 (572.16 mg, 774.48 umol, 0.05 eq) at 20° C. under N₂. The mixture was stirred at 110° C. for 3 hours. The reaction mixture was poured into water (50 mL). The aqueous phase was extracted with ethyl acetate (3×80 mL). The combined organic phase was washed with brine (2×100 mL), dried with anhydrous Na₂SO₄, filtered, and concentrated in vacuo. The crude product was purified by column chromatography (SiO₂, petroleum ether:ethyl acetate=20:1 to 5:1) to give B22-3 (5 g, 12.03 mmol, 77.70% yield) as a light-yellow solid. ¹H NMR (400 MHz, CDCl₃) δ 8.80 (d, J=6.0 Hz, 1H), 8.17 (d, J=2.8 Hz, 1H), 7.61 (ddd, J=3.8, 6.6, 8.4 Hz, 1H), 7.26 (br s, 1H), 6.89-6.56 (m, 2H), 6.22 (s, 2H), 4.68-4.55 (m, 2H), 4.12-3.99 (m, 2H), 3.53 (s, 2H), 2.32-2.18 (m, 2H), 2.05 (s, 3H).

Step 4: Synthesis of (5,22-difluoro-8,12-dioxa-18,20,24-triazatetracyclo[17.3.1.113,17.02,7]tetracosa-1(22),2,4,6,13,15,17(24),19(23),20-nonaen-15-yl)methyl-imino-methyl-oxo-λ⁶-sulfane (B22-4) (racemic mixture)

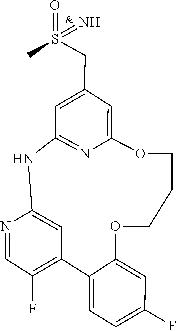 [SMILES: C[S@](=N)(=O)Cc1cc2nc(c1)OCCCOc1cc(F)ccc1-c1cc(ncc1F)N2]

To a mixture of B22-3 (400 mg, 962.80 umol, 1 eq) in DCM (10 mL) was added ammonia:carbamic acid (751.66 mg, 9.63 mmol, 10 eq) and PhI(OAc)₂ (775.28 mg, 2.41 mmol, 2.5 eq) at 20° C. under N₂. The mixture was stirred at 20° C. for 16 hours. 12 reactions were conducted in parallel. The reaction mixture was poured into water (50 mL). The aqueous phase was extracted with DCM (3×80 mL). The combined organic phase was washed with brine (2×80 mL), dried with anhydrous Na₂SO₄, filtered, and concentrated in vacuo. The reaction mixture was purified by column chromatography (SiO₂, petroleum ether/ethyl acetate=1:1 to ethyl acetate:MeOH=50:1) to give B22-4 (2.1 g, 4.47 mmol, 38.6% yield, 95% purity) as a light-yellow solid. ¹H NMR (400 MHz, DMSO-d₆) δ 9.71 (s, 1H), 8.69 (s, 1H), 8.31 (d, J=2.4 Hz, 1H), 7.57 (br s, 1H), 7.08 (dd, J=2.2, 11.4 Hz, 1H), 6.90 (dt, J=2.4, 8.4 Hz, 1H), 6.58 (s, 1H), 6.26 (s, 1H), 4.61-4.41 (m, 2H), 4.34-4.22 (m, 2H), 4.19-4.05 (m, 2H), 3.73 (s, 1H), 2.87 (s, 3H), 2.10 (br d, J=6.0 Hz, 2H).

Step 5: Chiral Separation of B22-4 to Give Building Block 22 and Building Block 23

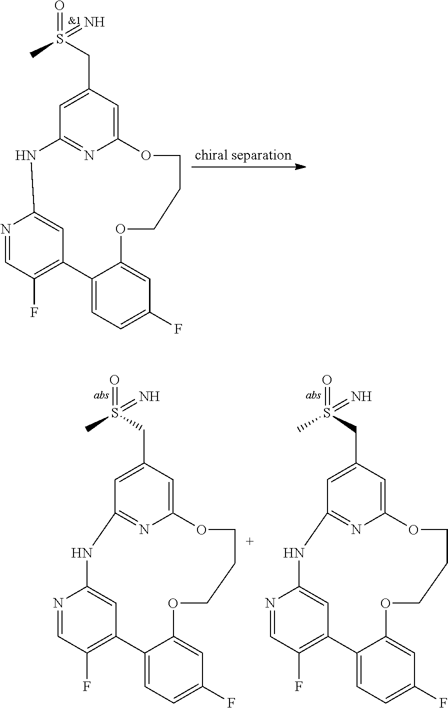 [SMILES: C[S@](=N)(=O)Cc1cc2nc(c1)OCCCOc1cc(F)ccc1-c1cc(ncc1F)N2 ; C[S@](=N)(=O)Cc1cc2nc(c1)OCCCOc1cc(F)ccc1-c1cc(ncc1F)N2 ; C[S@@](=N)(=O)Cc1cc2nc(c1)OCCCOc1cc(F)ccc1-c1cc(ncc1F)N2]

B22-4 was separated by prep-SFC(column: DAICEL CHIRALCEL OJ(250 mm*50 mm, 10 um); mobile phase: [0.1% NH₃H₂O ETOH]; B %: 60%-60%, 14 min, Rₜ1=1.67, Rₜ2=2.183) to give Building block 22 (750 mg, 1.52 mmol, 35.4% yield, 99.18% purity) as an off-white solid and Building block 23 (810 mg, 1.62 mmol, 37.7% yield, 97.77% purity) as a light-yellow solid. Note: The R/S configuration of these 2 compounds were not confirmed.

Building block 22: ¹H NMR (400 MHz, DMSO-d₆) δ 9.71 (s, 1H), 8.69 (d, J=6.0 Hz, 1H), 8.31 (d, J=2.4 Hz, 1H), 7.57 (dt, J=2.8, 4.2 Hz, 1H), 7.07 (dd, J=2.2, 11.4 Hz, 1H), 6.89 (dt, J=2.4, 8.4 Hz, 1H), 6.58 (s, 1H), 6.26 (s, 1H), 4.59-4.42 (m, 2H), 4.28 (d, J=2.2 Hz, 2H), 4.19-4.05 (m, 2H), 3.74 (s, 1H), 2.88 (s, 3H), 2.09 (br d, J=6.2 Hz, 2H). The desired enantiomer (optical rotation −4.88°±0.00°, 20 C, 589 nm) was obtained with 100% ee at Rₜ 1.63-1.83 min. LC-MS: Rₜ=2.38 min; MS (ESIpos): m/z=447 [M+H]⁺. Single (−) isomer, absolute stereochemistry unknown.

Building block 23: ¹H NMR (400 MHz, DMSO-d₆) δ 9.70 (s, 1H), 8.68 (br d, J=5.8 Hz, 1H), 8.34-8.27 (m, 1H), 7.62-7.52 (m, 1H), 7.07 (br d, J=9.8 Hz, 1H), 6.94-6.84 (m, 1H), 6.58 (s, 1H), 6.26 (s, 1H), 4.59-4.41 (m, 2H), 4.38-4.19 (m, 2H), 4.11 (br s, 2H), 3.74 (s, 1H), 2.88 (s, 3H), 2.18-2.01 (m, 2H). The desired enantiomer (optical rotation 3.860°±0.00°, 20 C, 589 nm) was obtained with 100% ee at Rₜ 2.05-2.42 min. LC-MS: Rₜ=2.38 min; MS (ESIpos): m/z=447 [M+H]⁺. Single (+) isomer, absolute stereochemistry unknown.

Example B24, Example B25, Example B26 & Example B27: Preparation of [(13R)-5,24-difluoro-13-methyl-8,14-dioxa-20,22,26-triazatetracyclo[19.3.1.115,19.02,7]hexacosa-1(24),2,4,6,15,17,19(26),21(25),22-nonaen-17-yl]methyl-imino-methyl-oxo-λ⁶-sulfane (four single isomers) (Building block 24, Building block 25, Building block 26, Building block 27)

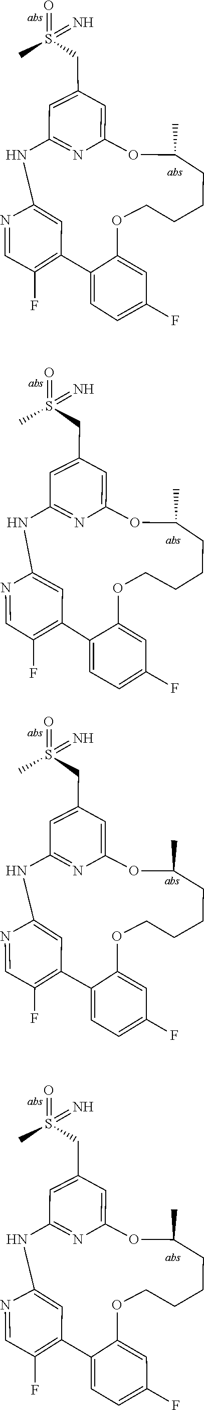

Building block 24, building block 25, building block 26 and building block 27 were prepared from commercially available intermediates according to the following steps.

Step 1: Synthesis of hexane-1,5-diol (B24-1)

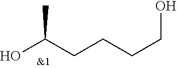

To a suspension of LiAlH₄ (19.95 g, 525.66 mmol, 1.5 eq) in THE (250 mL) was added a solution of 6-methyltetrahydropyran-2-one (40 g, 350.44 mmol, 1 eq) in THE (150 mL) at 0° C. under N₂. The mixture was stirred at 20° C. for 16 hours. The reaction mixture was quenched by the addition of NH₄C₁ (100 mL). The aqueous phase was extracted with 2-MeTHF (3×150 mL). The organic layer was washed with brine (2×100 mL), dried over Na₂SO₄, filtered, and concentrated under reduced pressure to give a crude product. The crude product was purified by column chromatography (SiO₂, petroleum ether/ethyl acetate=1:1 to 0:1) to give B24-1 (38 g, 321.56 mmol, 91.7% yield) as a light-yellow oil. ¹H NMR (400 MHz, CDCl₃) δ 3.88-3.69 (m, 1H), 3.61 (t, J=6.2 Hz, 2H), 2.69 (s, 2H), 1.62-1.36 (m, 6H), 1.17 (d, J=6.2 Hz, 3H).

Step 2: Synthesis of 6-[tert-butyl(diphenyl)silyl]oxyhexan-2-ol (B24-2)

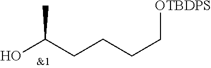

To a mixture of B24-1 (54 g, 456.95 mmol, 1 eq) in DCM (250 mL) was added imidazole (62.22 g, 913.91 mmol, 2 eq) and a solution of TBDPSCl (138.16 g, 502.65 mmol, 129.12 mL, 1.1 eq) in DCM (50 mL) at 0° C. The mixture was stirred at 15° C. for 1 hour. The reaction mixture was poured into water (100 mL). The aqueous phase was extracted with DCM (3×150 mL). The organic layer was washed with brine (2×100 mL), dried over Na₂SO₄, filtered, and concentrated under reduced pressure to give a crude product. The crude product was purified by column chromatography (SiO₂, petroleum ether:ethyl acetate=10:1 to 2:1) to give B24-2 (122 g, 334.55 mmol, 73.2% yield, 97.7% purity) as a colorless oil. ¹H NMR (400 MHz, CDCl₃) δ 7.69 (s, 4H), 7.52-7.33 (m, 6H), 3.83-3.76 (m, 1H), 3.73-3.67 (m, 2H), 1.66-1.54 (m, 2H), 1.53-1.38 (m, 5H), 1.20 (d, J=6.0 Hz, 3H), 1.09 (s, 9H).

Step 3: Synthesis of 6-[tert-butyl(diphenyl)silyl]oxyhexan-2-ol (B24-3)

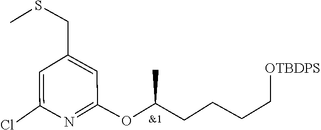

To a solution of B24-2 (30.84 g, 86.49 mmol, 1.5 eq) in THE (120 mL) was added NaH (3.46 g, 86.49 mmol, 60% purity, 1.5 eq) at 0° C. under N₂. The mixture was stirred at 20° C. for 30 minutes, then 2,6-dichloro-4-(methylsulfanylmethyl)pyridine (12 g, 57.66 mmol, 1.00 eq) in THE (25 mL) was added to the reaction mixture at 20° C. under N₂. The mixture was stirred at 70° C. for 15.5 h. The desired MS was detected by LCMS. The reaction mixture was poured into NH₄C₁ (100 mL). The aqueous phase was extracted with ethyl acetate (3×150 mL). The combined organic phase was washed with brine (2×100 mL), dried over anhydrous Na₂SO₄, filtered and concentrated in vacuo. The crude product was purified by column chromatography (SiO₂, petroleum ether/ethyl acetate=26:1 to 23:1) to give B24-3 (47 g, 88.98 mmol, 77.1% yield) as a colorless liquid.

¹H NMR (400 MHz, CDCl₃) δ 7.70 (br d, J=7.2 Hz, 4H), 7.49-7.33 (m, 6H), 6.88 (s, 1H), 6.54 (s, 1H), 5.26-5.07 (m, 1H), 3.70 (br t, J=6.2 Hz, 2H), 3.62-3.45 (m, 2H), 2.04 (s, 3H), 1.86-1.67 (m, 1H), 1.67-1.40 (m, 5H), 1.40-1.24 (m, 3H), 1.08 (s, 9H).

Step 4: Synthesis of 5-[[6-chloro-4-(methylsulfanylmethyl)-2-pyridyl]oxy]hexan-1-ol (B24-4)

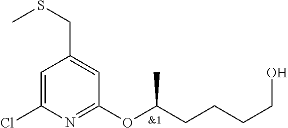 [SMILES: CSCc1cc(Cl)nc(O[C@@H](C)CCCCO)c1]

To a mixture of B24-3 (36.4 g, 68.91 mmol, 1 eq) in THE (150 mL) was added TBAF (1 M, 72.36 mL, 1.05 eq) at 20° C. under N₂. The mixture was stirred at 20° C. for 2 hours. TLC showed that the starting material had been consumed and that one new spot was formed. The desired MS was detected by LCMS. The reaction mixture was poured into water (30 mL). The aqueous phase was extracted with ethyl acetate (3×50 mL). The organic layer was washed with brine (2×40 mL), dried over Na₂SO₄, filtered, and concentrated under reduced pressure to give a crude product. The crude product was purified by column chromatography (SiO₂, petroleum ether:ethyl acetate=50:1 to 10:1) to give B24-4 (17.4 g, 60.04 mmol, 87.1% yield) as a white liquid. ¹H NMR (400 MHz, CDCl₃) δ 6.83 (s, 1H), 6.51 (s, 1H), 5.17 (s, 1H), 3.64 (t, J=6.4 Hz, 2H), 3.55-3.45 (m, 2H), 2.01 (s, 3H), 1.84-1.68 (m, 1H), 1.65-1.38 (m, 5H), 1.30 (d, J=6.2 Hz, 3H).

Step 5: Synthesis of 4-[2-[5-[[6-chloro-4-(methylsulfanylmethyl)-2-pyridyl]oxy]hexoxy]-4-fluoro-phenyl]-5-fluoro-pyridin-2-amine (B24-5)

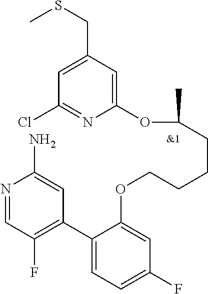 [SMILES: CSCc1cc(Cl)nc(O[C@@H](C)CCCCOc2cc(F)ccc2-c2cc(N)ncc2F)c1]

To a mixture of B24-4 (15 g, 51.76 mmol, 1 eq) and 2-(2-amino-5-fluoro-4-pyridyl)-5-fluoro-phenol (11.50 g, 51.76 mmol, 1 eq) in toluene (50 mL) was added CMBP (22.48 g, 93.16 mmol, 1.8 eq) at 20° C. under N₂. The mixture was stirred at 110° C. for 16 hours. The reaction mixture was poured into water (50 mL). The aqueous phase was extracted with ethyl acetate (3×100 mL). The combined organic phase was washed with brine (2×100 mL), dried with anhydrous Na₂SO₄, filtered, and concentrated in vacuo. The crude product was purified by column chromatography (SiO₂, petroleum ether:ethyl acetate=10:1 to 2:1) to give B24-5 (13.2 g, 25.92 mmol, 50.0% yield) as a white solid. ¹H NMR (400 MHz, CDCl₃) δ 7.94 (d, J=2.0 Hz, 1H), 7.21 (dd, J=7.2, 7.8 Hz, 1H), 6.89-6.81 (m, 1H), 6.77-6.63 (m, 2H), 6.52 (s, 1H), 6.47 (d, J=4.8 Hz, 1H), 5.23-5.12 (m, 1H), 4.37 (br s, 2H), 3.96 (t, J=6.2 Hz, 2H), 3.53 (s, 2H), 2.02 (s, 3H), 1.80-1.66 (m, 3H), 1.66-1.41 (m, 3H), 1.33-1.22 (m, 3H).

Step 6: Synthesis of 5,24-difluoro-13-methyl-17-(methylsulfanylmethyl)-8,14-dioxa-20,22,26-triazatetracyclo[19.3.1.1{15,19}0.0{2,7}]hexacosa-1(24),2,4,6,15,17,19(26),21(25),22-nonaene (B24-6)

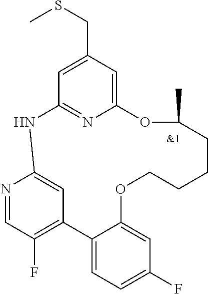 [SMILES: CSCc1cc2nc(c1)O[C@@H](C)CCCCOc1cc(F)ccc1-c1cc(ncc1F)N2]

To a solution of B24-5 (10 g, 20.24 mmol, 1 eq) in toluene (100 mL) and NMP (10 mL) was added XPhos (965.02 mg, 2.02 mmol, 0.1 eq), K₃PO₄ (21.48 g, 101.22 mmol, 5 eq) and [2-(2-aminoethyl)phenyl]-chloro-palladium;dicyclohexyl-[2-(2,4,6-triisopropylphenyl)phenyl]phosphane (747.74 mg, 1.01 mmol, 0.05 eq) at 20° C. under N₂. The mixture was stirred at 110° C. for 3 hours. The reaction mixture was poured into water (50 mL). The aqueous phase was extracted with ethyl acetate (3×100 mL). The combined organic phase was washed with brine (2×100 mL), dried over anhydrous Na₂SO₄, filtered, and concentrated in vacuo. The crude product was purified by column chromatography (SiO₂, petroleum ether/ethyl acetate=10:1 to 2:1) to give B24-6 (6.2 g, 13.55 mmol, 66.9% yield). ¹H NMR (400 MHz, CDCl₃) δ 8.25 (br s, 1H), 8.18-8.09 (m, 1H), 7.34-7.28 (m, 1H), 7.23 (br s, 1H), 6.82-6.72 (m, 2H), 6.25 (br d, J=12.4 Hz, 1H), 5.12-5.05 (m, 1H), 4.16-4.08 (m, 2H), 3.53 (s, 2H), 2.05 (s, 3H), 1.90-1.69 (m, 4H), 1.65-1.39 (m, 2H), 1.31-1.20 (m, 3H)

Step 7: Synthesis of (5,24-difluoro-13-methyl-8,14-dioxa-20,22,26-triazatetracyclo[19.3.1.115,19.02,7]hexacosa-1(24),2,4,6,15,17,19(26),21(25),22-nonaen-17-yl)methyl-imino-methyl-oxo-λ⁶-sulfane (B24-7)

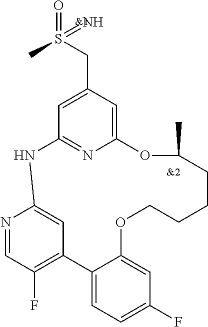 [SMILES: C[C@H]1CCCCOc2cc(F)ccc2-c2cc(ncc2F)Nc2cc(C[S@@](C)(=N)=O)cc(n2)O1]

B24-6 (500 mg, 1.09 mmol, 1 eq), PhI(OAc)₂ (1.76 g 5.46 mmol, 5 eq) was added to ammonia;carbamic acid (853.16 mg, 10.93 mmol, 10 eq) in i-PrOH (10 mL) at 20° C., then the reaction mixture was stirred at 25° C. for 20 hours. 26 reactions were conducted in parallel. The reaction mixture was poured into water (50 mL). The aqueous phase was extracted with DCM (3×100 mL). The combined organic phase was washed with brine (2×100 mL), dried over anhydrous Na₂SO₄, filtered, and concentrated in vacuo. The crude product was purified by column chromatography (SiO₂, ethyl acetate/methanol=100:1 to 50:1) to give B24-7 (8.1 g, 16.08 mmol, 58.87% yield, 97% purity). ¹H NMR (400 MHz, CDCl₃) δ 8.24 (d, J=5.6 Hz, 1H), 8.16 (d, J=1.4 Hz, 1H), 7.38 (s, 1H), 7.33-7.27 (m, 1H), 6.84-6.73 (m, 2H), 6.34 (s, 1H), 6.30-6.20 (m, 1H), 5.14-5.05 (m, 1H), 4.17-4.04 (m, 4H), 3.00 (s, 3H), 1.94-1.71 (m, 4H), 1.55-1.38 (m, 2H), 1.30-1.22 (m, 3H)

Step 8: Chiral Separation of B24-7 to Give Building Block 24 (B24), Building Block 25 (B25), Building Block 26 (B26) and Building Block 27 (B27)

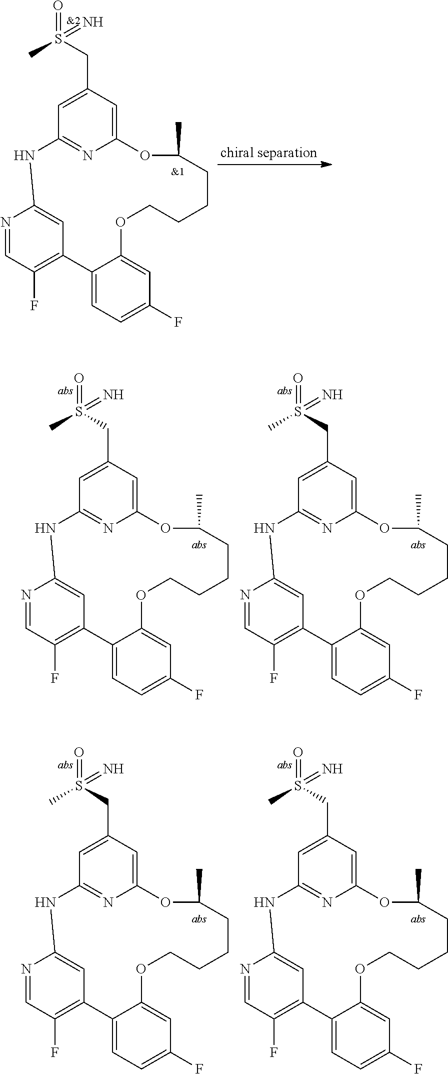

B24-7 was separated by prep-SFC (column: DAICEL CHIRALPAK AD(250 mm*50 mm, 10 um);mobile phase: [0.1% NH₃H₂O IPA];B %: 50%-50%, 7.1 min) to give mixture A (B24 and B25) and mixture B (B26 and B27).

Mixture A (B24 and B25) was separated by prep-SFC (column: DAICEL CHIRALPAK AD(250 mm*50 mm, 10 um);mobile phase: [0.10% NH₃H₂O MEOH];B %: 65%-65%, 25 min) to give Building block 24 (583.28 mg, 1.17 mmol, 7.15% yield, 98.06% purity) as a light-yellow solid, and Building block 25 (604.06 mg, 1.22 mmol, 7.46% yield, 98.86% purity) as a light-yellow solid.

Mixture B (B26 and B27) was separated by prep-SFC (column: DAICEL CHIRALCEL OD(250 mm*30 mm, 10 um);mobile phase: [0.1% NH₃H₂O IPA];B %: 30%-30%, 9 min) to give Building block 26 (632.14 mg, 1.25 mmol, 7.64% yield, 96.66% purity) as a light-yellow solid, and Building block 27 (699.91 mg, 1.41 mmol, 8.62% yield, 98.52% purity) as a light-yellow solid. Note: The exact configuration of these 4 compounds were not confirmed.

Building block 24: ¹H NMR (400 MHz, DMSO-d₆) δ 9.70 (s, 1H), 8.32-8.26 (m, 1H), 8.19 (d, J=5.4 Hz, 1H), 7.38-7.28 (m, 1H), 7.20 (br d, J=2.2 Hz, 1H), 6.89 (dt, J=2.2, 8.4 Hz, 1H), 6.62 (s, 1H), 6.28 (s, 1H), 4.89-4.79 (m, 1H), 4.31-4.20 (m, 3H), 4.20-4.02 (m, 1H), 3.75 (s, 1H), 2.87 (s, 3H), 1.78-1.52 (m, 4H), 1.49-1.32 (m, 2H), 1.19 (d, J=6.2 Hz, 3H). The desired enantiomer (optical rotation 136.770±0.00°, 20 C, 589 nm) was obtained with 100% ee at Rₜ 1.50-2.55 min. LC-MS: Rₜ=2.54 min; MS (ESIpos): m/z=489 [M+H]⁺. Single (+) isomer, absolute stereochemistry unknown.

Building block 25: ¹H NMR (400 MHz, DMSO-d₆) δ 9.70 (s, 1H), 8.29 (s, 1H), 8.19 (d, J=5.6 Hz, 1H), 7.39-7.27 (m, 1H), 7.20 (br d, J=2.0 Hz, 1H), 6.89 (dt, J=2.2, 8.4 Hz, 1H), 6.62 (s, 1H), 6.28 (s, 1H), 4.90-4.80 (m, 1H), 4.32-4.19 (m, 3H), 4.19-4.01 (m, 1H), 3.75 (s, 1H), 2.87 (s, 3H), 1.77-1.54 (m, 4H), 1.50-1.31 (m, 2H), 1.19 (d, J=6.2 Hz, 3H). The desired enantiomer (optical rotation 125.73°±0.00°, 20 C, 589 nm) was obtained with 98.8% ee at Rₜ 2.50-4.20 min. LC-MS: Rₜ=2.54 min; MS (ESIpos): m/z=489 [M+H]⁺. Single (+) isomer, absolute stereochemistry unknown.

Building block 26: ¹H NMR (400 MHz, DMSO-d₆) δ 9.72 (s, 1H), 8.29 (s, 1H), 8.19 (d, J=5.4 Hz, 1H), 7.40-7.29 (m, 1H), 7.19 (dd, J=2.0, 11.6 Hz, 1H), 6.89 (dt, J=2.2, 8.2 Hz, 1H), 6.62 (s, 1H), 6.28 (s, 1H), 4.96-4.76 (m, 1H), 4.32-4.19 (m, 3H), 4.16-4.03 (m, 1H), 3.75 (s, 1H), 2.87 (s, 2H), 1.72 (br s, 4H), 1.52-1.27 (m, 2H), 1.25-1.08 (m, 3H). The desired enantiomer (optical rotation −123.15°±0.00°, 20 C, 589 nm) was obtained with 100% ee at Rt 2.35-2.80 min. LC-MS: Rₜ=2.54 min; MS (ESIpos): m/z=489 [M+H]⁺. Single (−) isomer, absolute stereochemistry unknown.

Building block 27: ¹H NMR (400 MHz, DMSO-d₆) δ 9.72 (s, 1H), 8.29 (s, 1H), 8.19 (d, J=5.6 Hz, 1H), 7.31 (s, 1H), 7.21-7.14 (m, 1H), 7.27-7.12 (m, 1H), 6.89 (dt, J=2.4, 8.2 Hz, 1H), 6.62 (s, 1H), 6.28 (s, 1H), 4.92-4.75 (m, 1H), 4.30-4.16 (m, 3H), 4.14-4.03 (m, 1H), 3.82-3.75 (m, 1H), 2.87 (s, 3H), 1.77-1.54 (m, 4H), 1.51-1.31 (m, 2H), 1.23-1.12 (m, 3H). The desired enantiomer (optical rotation −138.54°±0.28°, 20 C, 589 nm) was obtained with 99.74% ee at Rₜ 2.85-3.40 min. LC-MS: Rₜ=2.54 min; MS (ESIpos): m/z=489 [M+H]⁺. Single (−) isomer, absolute stereochemistry unknown.

Example B28, Example B29: Preparation of (3,20-difluoro-13-oxa-5,7,18,25-tetrazatetracyclo[17.3.1.12,6.18,12]pentacosa-1(22),2,4,6(25),8,10,12(24),19(23),20-nonaen-10-yl)methyl-imino-methyl-oxo-λ⁶-sulfane (two single enantiomers) (Building block 28, Building block 29)

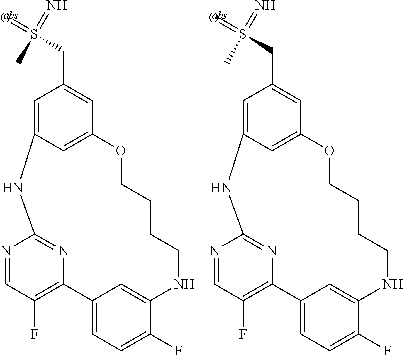

Building block 28 and 29 were prepared from commercially available intermediates according to the following steps.

Step 1: Synthesis of tert-butyl N-[[3-amino-5-(4-hydroxybutoxy)phenyl]methyl-methyl-oxo-λ⁶-sulfanylidene]carbamate (B28-1)

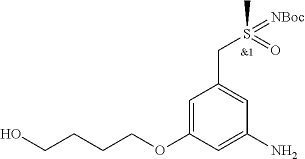

To a mixture of tert-butyl N-[[3-(4-hydroxybutoxy)-5-nitro-phenyl]methyl-methyl-oxo-λ⁶-sulfanylidene]carbamate (4 g, 9.94 mmol, 1 eq) in EtOH (60 mL) and H₂O (12 mL) was added Fe (2.78 g, 49.69 mmol, 5 eq) and NH₄C₁ (2.66 g, 49.69 mmol, 5 eq) at 20° C. under N₂. The mixture was stirred at 80° C. for 1 hour. The reaction mixture was filtered and concentrated under reduced pressure to remove EtOH. The mixture was diluted with H₂O (50 mL) and extracted with EtOAc (3×80 mL). The combined organic layers were washed with brine (2×80 mL), dried over anhydrous Na₂SO₄, filtered, and concentrated under reduced pressure to give B28-1 (3.8 g, crude) as a yellow solid. ¹H NMR (400 MHz, CDCl₃) δ 6.32-6.29 (m, 2H), 6.24 (t, J=2.0 Hz, 1H), 4.59 (s, 2H), 3.94 (t, J=6.2 Hz, 2H), 3.70 (t, J=6.2 Hz, 2H), 2.94 (s, 3H), 1.90-1.78 (m, 2H), 1.78-1.65 (m, 2H), 1.51 (s, 9H).

Step 2: Synthesis of 2-chloro-5-fluoro-4-(4-fluoro-3-nitro-phenyl)pyrimidine (B28-2)

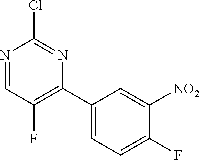 [SMILES: O=[N+]([O-])c1cc(-c2nc(Cl)ncc2F)ccc1F]

To a solution of 2,4-dichloro-5-fluoro-pyrimidine (9.48 g, 56.78 mmol, 1 eq) and (4-fluoro-3-nitro-phenyl)boronic acid (10.5 g, 56.78 mmol, 1 eq) in DME (150 mL) were added K₂CO₃ (2 M, 85.17 mL, 3 eq) and Pd(dppf)Cl₂·CH₂Cl₂ (4.64 g, 5.68 mmol, 0.1 eq) at 20° C. under N₂. The mixture was stirred at 80° C. for 2.5 hours. TLC showed that the starting material had disappeared and that a new main spot was formed. The mixture was extracted with EtOAc (2×100 mL) and H₂O (100 mL). The combined organic phase was washed with brine (3×50 mL), dried with anhydrous Na₂SO₄, filtered, and concentrated in vacuo. The residue was purified by column chromatography (SiO₂, petroleum ether:ethyl acetate=20:1 to 8:1) to give B28-2 (27 g, 99.41 mmol, 87.5% yield) as a light-yellow solid.

¹H NMR (400 MHz, DMSO-d₆) δ 9.06 (d, J=3.0 Hz, 1H), 8.73 (dd, J=2.2, 7.2 Hz, 1H), 8.47-8.39 (m, 1H), 7.83 (dd, J=8.8, 11.2 Hz, 1H).

Step 3: Synthesis of 5-(2-chloro-5-fluoro-pyrimidin-4-yl)-2-fluoro-aniline (B28-3)

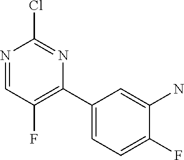 [SMILES: Nc1cc(-c2nc(Cl)ncc2F)ccc1F]

To a solution of B28-2 (19.7 g, 72.53 mmol, 1 eq) in EtOH (200 mL) and H₂O (40 mL) were added Fe (20.25 g, 362.66 mmol, 5 eq) and NH₄C₁ (19.40 g, 362.66 mmol, 5 eq) at 20° C. The mixture was stirred at 80° C. for 2 hours. The mixture was filtered through a pad of Celite, and the pad cake was washed with EtOH (5×15 mL). The mixture was concentrated under reduced pressure to remove EtOH. The mixture was diluted with H₂O (100 mL) and extracted with ethyl acetate (3×100 mL). The combined organic layers were washed with brine (2×50 mL), dried over Na₂SO₄, filtered, and concentrated under reduced pressure to give a residue. The residue was purified by column chromatography (SiO₂, petroleum ether:ethyl acetate=20:1 to 8:1) to give B28-3 (12.4 g, 51.32 mmol, 70.75% yield) as a yellow solid. ¹H NMR (400 MHz, DMSO-d₆) δ 8.89 (d, J=3.6 Hz, 1H), 7.56 (dd, J=1.8, 8.8 Hz, 1H), 7.28-7.14 (m, 2H), 5.53 (s, 2H).

Step 4: Synthesis of N-[5-(2-chloro-5-fluoro-pyrimidin-4-yl)-2-fluoro-phenyl]-2-nitro-benzenesulfonamide (B28-4)

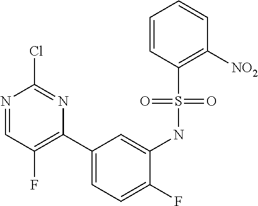 [SMILES: O=[N+]([O-])c1ccccc1S(=O)(=O)Nc1cc(-c2nc(Cl)ncc2F)ccc1F]

To a mixture of B28-3 (7.35 g, 30.42 mmol, 1 eq) and 2-nitrobenzenesulfonyl chloride (10.79 g, 48.67 mmol, 1.6 eq) in DCM (80 mL) was added DMAP (260.14 mg, 2.13 mmol, 0.07 eq) and pyridine (3.85 g, 48.67 mmol, 3.93 mL, 1.6 eq) at 20° C. under N₂. The mixture was stirred at 20° C. for 16 hours. The reaction mixture was poured into water (50 mL). The aqueous phase was extracted with ethyl acetate (3×100 mL). The combined organic phase was washed with brine (2×100 mL), dried over anhydrous Na₂SO₄, filtered, and concentrated in vacuo. The crude product was washed with DCM (2×8 mL) to give B28-4 (21 g, 49.21 mmol, 80.88% yield) as a yellow solid. ¹H NMR (400 MHz, CDCl₃) δ 8.58 (d, J=2.8 Hz, 1H), 8.49-8.41 (m, 1H), 8.05-7.93 (m, 3H), 7.73-7.62 (m, 3H), 7.18 (t, J=9.2 Hz, 1H).

Step 5: Synthesis of N-[5-(2-chloro-5-fluoro-pyrimidin-4-yl)-2-fluoro-phenyl]-2-nitro-benzenesulfonamide (B28-5)

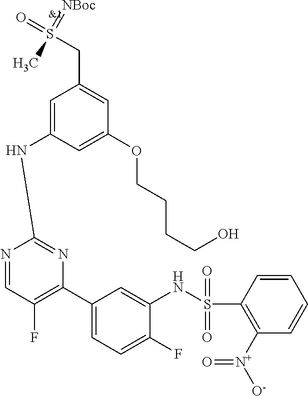

To a mixture of B28-1 (3.8 g, 10.20 mmol, 1 eq) and B28-4 (4.35 g, 10.20 mmol, 1 eq) in NMP (6 mL) and toluene (60 mL) was added Xphos Pd G1 (376.84 mg, 510.10 umol, 0.05 eq), Xphos (486.35 mg, 1.02 mmol, 0.1 eq) and K₃PO₄ (10.83 g, 51.01 mmol, 5 eq) at 20° C. under N₂. The mixture was stirred at 110° C. for 4 hours. TLC (petroleum ether:ethyl acetate=0:1, R_{f}=0.4) indicated that the starting material was consumed completely and that one new spot formed. The reaction mixture was poured into water (50 mL). The aqueous phase was extracted with ethyl acetate (2×100 mL). The combined organic phase was washed with brine (2×100 mL), dried over anhydrous Na₂SO₄, filtered, and concentrated in vacuo. The residue was purified by column chromatography (SiO₂, petroleum ether:ethyl acetate=20:1 to 1:1) to give B28-5 (6.5 g, 8.52 mmol, 83.5% yield) as a yellow solid.

¹H NMR (400 MHz, CDCl₃) δ 8.43 (dd, J=2.2, 7.6 Hz, 1H), 8.39-8.33 (m, 1H), 8.01-7.86 (m, 3H), 7.79-7.68 (m, 1H), 7.67-7.57 (m, 1H), 7.50 (s, 1H), 7.38-7.31 (m, 2H), 7.13 (t, J=9.2 Hz, 1H), 6.64 (s, 1H), 4.81-4.63 (m, 2H), 4.03 (t, J=6.2 Hz, 2H), 3.72 (t, J=6.4 Hz, 2H), 3.02 (s, 3H), 1.94-1.84 (m, 2H), 1.79-1.71 (m, 2H), 1.48 (s, 9H)

Step 6: Synthesis of tert-butyl N-[[3,20-difluoro-18-(2-nitrophenyl)sulfonyl-13-oxa-5,7,18,25-tetrazatetracyclo[17.3.1.12,6.18,12]pentacosa-1(22),2,4,6(25),8,10,12(24),19(23),20-nonaen-10-yl]methyl-methyl-oxo-λ⁶-sulfanylidene]carbamate (B28-6)

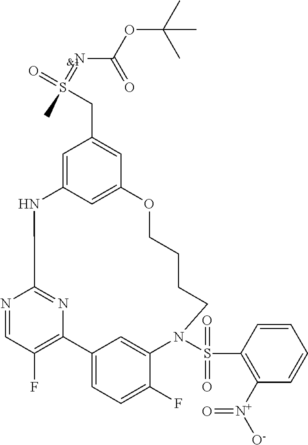 [SMILES: CC(C)(C)OC(=O)N=[S@@](C)(=O)Cc1cc2cc(c1)OCCCCN(S(=O)(=O)c1ccccc1[N+](=O)[O-])c1cc(ccc1F)-c1nc(ncc1F)N2]

To a mixture of B28-5 (5.4 g, 7.08 mmol, 1 eq) in toluene (110 mL) was added CMBP (3.42 g, 14.16 mmol, 2 eq) at 20° C. under N₂. The mixture was stirred at 110° C. for 16 hours. The reaction mixture was diluted with EtOAc (30 mL) and filtered, the solid was dried in vacuo to give B28-6 (4.6 g, 6.18 mmol, 87.2% yield) as a white solid. ¹H NMR (400 MHz, DMSO-d₆) δ 10.03 (s, 1H), 8.74 (d, J=3.6 Hz, 1H), 8.32 (br d, J=5.8 Hz, 1H), 8.15 (br d, J=4.4 Hz, 1H), 8.07 (s, 1H), 7.98-7.95 (m, 1H), 7.89 (t, J=7.6 Hz, 1H), 7.84-7.81 (m, 1H), 7.79-7.74 (m, 1H), 7.49 (t, J=9.4 Hz, 1H), 6.85 (s, 1H), 6.60 (s, 1H), 4.78-4.70 (m, 2H), 4.05-3.91 (m, 2H), 3.91-3.68 (m, 2H), 3.17 (s, 3H), 1.92-1.72 (m, 2H), 1.39 (s, 9H), 1.36-1.22 (m, 2H).

Step 7: Synthesis of tert-butyl N-[(3,20-difluoro-13-oxa-5,7,18,25-tetrazatetracyclo[17.3.1.12,6.18,12]pentacosa-1(22),2,4,6(25),8,10,12(24),19(23),20-nonaen-10-yl)methyl-methyl-oxo-λ⁶-sulfanylidene]carbamate (B28-7)

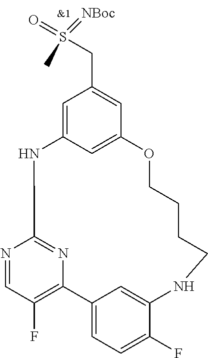

To a solution of B28-6 (4.6 g, 6.18 mmol, 1 eq) in DMF (80 mL) was added phenylsulfanylsodium (1.8 g, 13.59 mmol, 2.2 eq) at 20° C. under N₂. The mixture was stirred at 20° C. for 16 hours. The reaction mixture was poured into water (50 mL). The aqueous phase was extracted with dichloromethane (2×100 mL). The combined organic phase was washed with brine (2×100 mL), dried over anhydrous Na₂SO₄, filtered, and concentrated in vacuo. The residue was purified by column chromatography (SiO₂, petroleum ether/ethyl acetate=10:1 to 0:1) to give B28-7 (2.85 g, 4.77 mmol, 77.1% yield, 93.6% purity) as a light-yellow solid. ¹H NMR (400 MHz, DMSO-d₆) δ 9.87 (s, 1H), 8.59 (d, J=4.2 Hz, 1H), 8.08 (s, 1H), 7.64 (br d, J=7.8 Hz, 1H), 7.30-7.22 (m, 1H), 7.16 (dd, J=8.4, 11.4 Hz, 1H), 6.89 (s, 1H), 6.84-6.74 (m, 1H), 5.96 (br s, 1H), 4.77 (s, 2H), 4.13 (br t, J=4.8 Hz, 2H), 3.25-3.10 (m, 5H), 1.80 (br dd, J=7.8, 16.2 Hz, 2H), 1.71-1.54 (m, 2H), 1.39 (s, 9H).

Step 8: Synthesis of (3,20-difluoro-13-oxa-5,7,18,25-tetrazatetracyclo[17.3.1.12,6.18,12]pentacosa-1(22),2,4,6(25),8,10,12(24),19(23),20-nonaen-10-yl)methyl-imino-methyl-oxo-λ⁶-sulfane (B28-8)

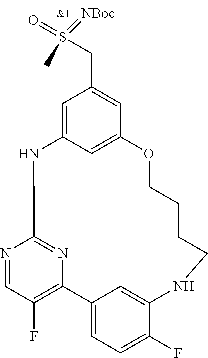

B28-7 (2.85 g, 5.09 mmol, 1 eq) was dissolved in TFA (3 mL) and DCM (30 mL) at 20° C. under N₂. The mixture was stirred at 20° C. for 16 hours. The reaction mixture was poured into NaHCO₃ (50 mL) and ethyl acetate (50 mL). The mixture was stirred for 0.5 hours, then the mixture was filtered, and the filter cake was dried in vacuo. The crude product was washed with EtOAc (30 mL) to give B28-8 (2.1 g, 4.30 mmol, 84.3% yield, 94% purity) as a light-yellow solid. ¹H NMR (400 MHz, DMSO-d₆) δ 9.81 (s, 1H), 8.59 (d, J=4.4 Hz, 1H), 8.03 (s, 1H), 7.66 (br d, J=7.6 Hz, 1H), 7.30-7.23 (m, 1H), 7.23-7.11 (m, 1H), 6.86 (s, 1H), 6.84-6.78 (m, 1H), 5.97 (br t, J=5.1 Hz, 1H), 4.37-4.24 (m, 2H), 4.14 (br t, J=4.8 Hz, 2H), 3.62 (s, 1H), 3.29-3.14 (m, 2H), 2.85 (s, 3H), 1.87-1.72 (m, 2H), 1.72-1.55 (m, 2H).

Step 9: Chiral separation of B28-8 to give Building block 28 and Building block 29

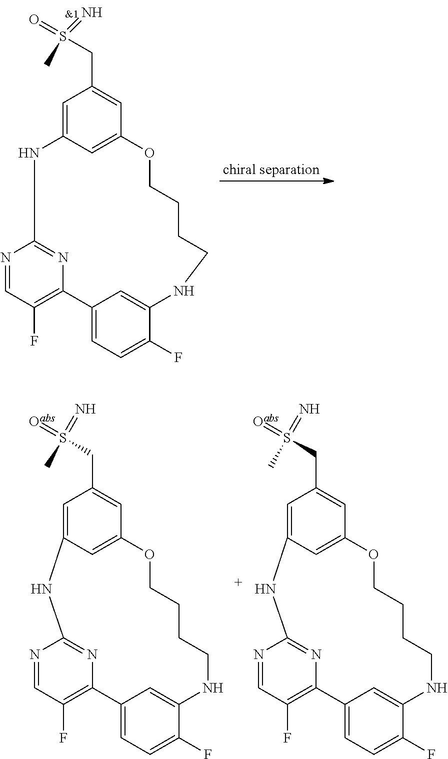 [SMILES: C[S@](=N)(=O)Cc1cc2cc(c1)OCCCCNc1cc(ccc1F)-c1nc(ncc1F)N2 ; C[S@](=N)(=O)Cc1cc2cc(c1)OCCCCNc1cc(ccc1F)-c1nc(ncc1F)N2 ; C[S@@](=N)(=O)Cc1cc2cc(c1)OCCCCNc1cc(ccc1F)-c1nc(ncc1F)N2]

Compound B28-8 was separated by prep-SFC (column: DAICEL CHIRALPAK AD (250 mm*30 mm, 10 um);mobile phase: [ACN/EtOH(0.1% NH₃H₂O)]; B %: 60%-60%, 45 min) to give B28 (803.38 mg, 38.2% yield) as a light yellow solid and B29 (762.33 mg, 36.3% yield) as a light-yellow solid. Note: The R/S configuration of these 2 compounds were not confirmed.

Building block 28: ¹H NMR (400 MHz, DMSO-d₆) δ 9.82 (s, 1H), 8.59 (d, J=4.4 Hz, 1H), 8.03 (t, J=1.8 Hz, 1H), 7.66 (dd, J=1.8, 8.8 Hz, 1H), 7.32-7.19 (m, 1H), 7.19-7.06 (m, 1H), 6.84 (br d, J=18.4 Hz, 2H), 6.02-5.92 (m, 1H), 4.39-4.20 (m, 2H), 4.20-4.08 (m, 2H), 3.61 (s, 1H), 3.26-3.11 (m, 2H), 2.84 (s, 3H), 1.86-1.68 (m, 2H), 1.67-1.52 (m, 2H). The desired enantiomer (optical rotation 3.540±0.00°, 20 C, 589 nm) was obtained with 100% ee at Rt 2.75-3.70 min. LC-MS: Rₜ=2.33 min; MS (ESIpos): m/z=460 [M+H]⁺. Single (+) isomer, absolute stereochemistry unknown.

Building block 29: ¹H NMR (400 MHz, DMSO-d₆) δ 9.81 (s, 1H), 8.59 (d, J=4.2 Hz, 1H), 8.03 (s, 1H), 7.65 (br d, J=7.4 Hz, 1H), 7.30-7.21 (m, 1H), 7.17 (dd, J=8.4, 11.6 Hz, 1H), 6.88-6.83 (m, 1H), 6.81 (s, 1H), 5.97 (br t, J=5.4 Hz, 1H), 4.37-4.21 (m, 2H), 4.21-4.06 (m, 2H), 3.61 (s, 1H), 3.28-3.14 (m, 2H), 2.84 (s, 3H), 1.95-1.73 (m, 2H), 1.66-1.52 (m, 2H). The desired enantiomer (optical rotation −4.33°±0.28°, 20 C, 589 nm) was obtained with 99.62% ee at Rₜ 3.70-4.80 min. LC-MS: Rₜ=2.33 min; MS (ESIpos): m/z=460 [M+H]⁺. Single (−) isomer, absolute stereochemistry unknown.

INTERMEDIATES

Example I1: Preparation of tert-butyl [(2S)-1-{[(R*)-[(3-{[4-(4-fluoro-2-methoxyphenyl)-1,3,5-triazin-2-yl]amino}phenyl) methyl](methyl)oxo-lambda⁶-sulfanylidene]amino}-3-methyl-1-oxobutan-2-yl]carbamate (Intermediate 1)

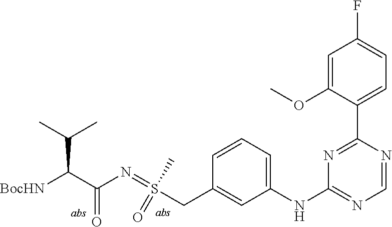 [SMILES: COc1cc(F)ccc1-c1ncnc(Nc2cccc(C[S@@](C)(=O)=NC(=O)[C@@H](NC(=O)OC(C)(C)C)C(C)C)c2)n1]

4-(4-Fluoro-2-methoxyphenyl)-N-{3-[(S-methanesulfonimidoyl)methyl]phenyl}-1,3,5-triazin-2-amine Building bock 4 (PT-1″) (70.0 mg, 181 μmol) and N-(tert-butoxycarbonyl)-L-valine (78.5 mg, 361 μmol) were dissolved in DMF (10 mL), 2.2 eq EDCI (76.2 mg, 397 μmol) and 2.5 eq HOBT hydrate (69.2 mg, 452 μmol) and 4.0 eq N,N-diisopropylethylamine (130 μl, 720 μmol) were added. After stirring overnight at rt the mixture was concentrated in vacuo and the crude product was purified by prep. HPLC then concentrated and lyophilized to yield Intermediate 1 (70.0 mg, 100% purity, 66% yield) as a light yellow solid. LC-MS: Rₜ=2.01 min; MS (ESIpos): m/z=587 [M+H]⁺.

Example I2: Preparation of trifluoroacetic acid-N—[(R*)-[(3-{[4-(4-fluoro-2-methoxyphenyl)-1,3,5-triazin-2-yl]amino} phenyl) methyl](methyl)oxo-lambda⁶-sulfanylidene]-L-valinamide (1/1) (Intermediate 2)

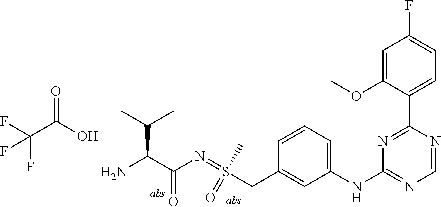 [SMILES: O=C(O)C(F)(F)F ; COc1cc(F)ccc1-c1ncnc(Nc2cccc(C[S@@](C)(=O)=NC(=O)[C@@H](N)C(C)C)c2)n1]

Tert-butyl [(2S)-1-{[(R*)-[(3-{[4-(4-fluoro-2-methoxyphenyl)-1,3,5-triazin-2-yl]amino}phenyl) methyl](methyl)oxo-lambda⁶-sulfanylidene]amino}-3-methyl-1-oxobutan-2-yl]carbamate (Intermediate 1) (70.0 mg, 119 μmol) were dissolved in DCM (8 mL), TFA (1 mL) was added and the reaction mixture was stirred for 30 min. It was concentrated in vacuo, dissolved in ACN/H₂O and lyophilized to yield Intermediate 2 in quantitative yield (74.0 mg, 100% purity, 103%). LC-MS: Rₜ=1.06 min; MS (ESIneg): m/z=485 [M−H].

Example I3: Preparation of tert-butyl (19S)-19-[(2S)-2-{[(2S)-1-{[(R*)-[(3-{[4-(4-fluoro-2-methoxyphenyl)-1,3,5-triazin-2-yl]amino}phenyl)methyl](methyl)oxo-lambda⁶-sulfanylidene]amino}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 3)

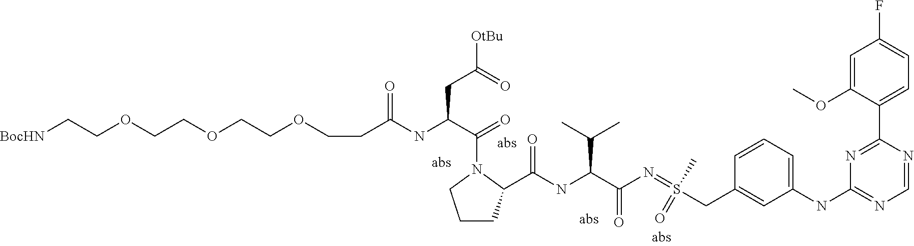 [SMILES: COc1cc(F)ccc1-c1ncnc(Nc2cccc(C[S@@](C)(=O)=NC(=O)[C@@H](NC(=O)[C@@H]3CCCN3C(=O)[C@H](CC(=O)OC(C)(C)C)NC(=O)CCOCCOCCOCCNC(=O)OC(C)(C)C)C(C)C)c2)n1]

Trifluoroacetic acid-N—[(R*)-[(3-{[4-(4-fluoro-2-methoxyphenyl)-1,3,5-triazin-2-yl]amino} phenyl)methyl](methyl)oxo-lambda⁶-sulfanylidene]-L-valinamide (1/1) (intermediate 2; 25.5 mg, 42.4 μmol) (Intermediate 2) and (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (Building block 3) (27.5 mg, 46.6 μmol) were dissolved in DMF (8 mL) and 1.5 eq EDCI (12.2 mg, 63.6 μmol), 1.6 eq HOBT hydrate (10.4 mg, 67.8 μmol) as well as 5.0 eq N,N-diisopropylethylamine (37 μl, 210 μmol) were added. After stirring for 3 h at rt, the mixture was concentrated in vacuo and the residual was purified by prep. HPLC then concentrated and lyophilized to give Intermediate 3 (32.0 mg, 94% purity, 67%).

Example I4: Preparation of N-(3-{2-[2-(2-aminoethoxy)ethoxy]ethoxy}propanoyl)-L-alpha-aspartyl-L-prolyl-N—[(R*)-[(3-{[4-(4-fluoro-2-methoxyphenyl)-1,3,5-triazin-2-yl]amino}phenyl)methyl](methyl)oxo-lambda⁶-sulfanylidene]-L-valinamide-trifluoroacetic acid (1/1) (Intermediate 4)

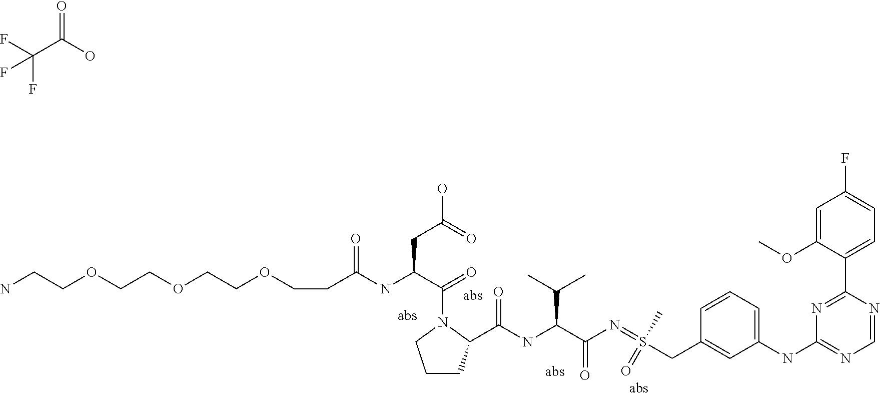 [SMILES: O=C(O)C(F)(F)F ; COc1cc(F)ccc1-c1ncnc(Nc2cccc(C[S@@](C)(=O)=NC(=O)[C@@H](NC(=O)[C@@H]3CCCN3C(=O)[C@H](CC(=O)O)NC(=O)CCOCCOCCOCCN)C(C)C)c2)n1]

Tert-butyl (19S)-19-[(2S)-2-{[(2S)-1-{[(R*)-[(3-{[4-(4-fluoro-2-methoxyphenyl)-1,3,5-triazin-2-yl]amino}phenyl)methyl](methyl)oxo-lambda⁶-sulfanylidene]amino}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 3) (32.0 mg, 28.4 μmol) was dissolved in DCM (5 mL), TFA (2 mL) was added, and the reaction mixture was stirred for 2 h. It was concentrated in vacuo, dissolved in ACN/H₂O and lyophilized to give Intermediate 4 in quantitative yield (33.0 mg, 93% purity). LC-MS: Rₜ=1.13 min; MS (ESIneg): m/z=900 [M−H]—.

Example I5: Preparation of tert-butyl [(2S)-1-{[(S)-[(2-{[5-fluoro-4-(4-fluoro-2-methoxyphenyl)pyridin-2-yl]amino}pyridin-4-yl)methyl](methyl)oxo-lambda⁶-sulfanylidene]amino}-3-methyl-1-oxobutan-2-yl]carbamate (Intermediate 5)

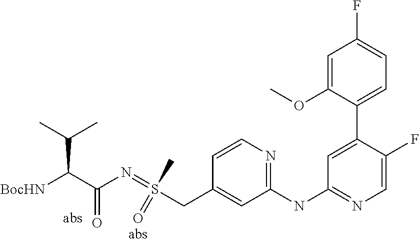 [SMILES: COc1cc(F)ccc1-c1cc(Nc2cc(C[S@](C)(=O)=NC(=O)[C@@H](NC(=O)OC(C)(C)C)C(C)C)ccn2)ncc1F]

(S)-5-Fluoro-4-(4-fluoro-2-methoxyphenyl)-N-{4-[(S-methylsulfonimidoyl)methyl]pyridin-2-yl}pyridin-2-amine (Building block 5 (PT-2′)) (100.0 mg, 247 μmol) and N-(tert-butoxycarbonyl)-L-valine (64.5 mg, 297 μmol) were dissolved in DMF (10 mL) and 1.5 eq HATU (141 mg, 371 μmol) as well as 3.0 eq N,N-diisopropylethylamine (130 μl, 740 μmol) were added. After stirring overnight at rt, the mixture was concentrated in vacuo and the residual was purified by prep. HPLC then concentrated in vacuo to yield Intermediate 5 as a colorless foam (115 mg, 100% purity, 77%). LC-MS: Rₜ=3.08 min; MS (ESIpos): m/z=604 [M+H]⁺.

Example I6: Preparation of trifluoroacetic acid-N—[(S)-[(2-{[5-fluoro-4-(4-fluoro-2-methoxyphenyl)pyridin-2-yl]amino} pyridin-4-yl)methyl](methyl)oxo-lambda⁶-sulfanylidene]-L-valinamide (1/1) (Intermediate 6)

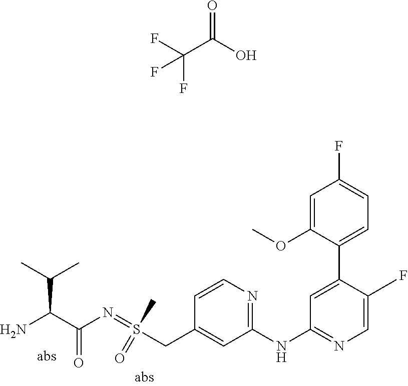 [SMILES: O=C(O)C(F)(F)F ; COc1cc(F)ccc1-c1cc(Nc2cc(C[S@](C)(=O)=NC(=O)[C@@H](N)C(C)C)ccn2)ncc1F]

Tert-butyl [(2S)-1-{[(S)-[(2-{[5-fluoro-4-(4-fluoro-2-methoxyphenyl)pyridin-2-yl]amino}pyridin-4-yl)methyl](methyl)oxo-lambda⁶-sulfanylidene]amino}-3-methyl-1-oxobutan-2-yl]carbamate (Intermediate 5) (115 mg, 190 μmol) was dissolved in DCM (10 mL), TFA (2 mL) was added and the reaction mixture was stirred for 30 min at rt. It was concentrated in vacuo, redissolved in ACN/H₂O and lyophilized to yield Intermediate 6 as a colorless foam in quantitative yield (120.0 mg, 100% purity, 100%). LC-MS: Rₜ=1.73 min; MS (ESIpos): m/z=503 [M+H]⁺.

Example I7: Preparation of tert-butyl (19S)-19-{[(2S)-2-{[(2S)-1-{[(S*)-[(2-{[5-fluoro-4-(4-fluoro-2-methoxyphenyl)pyridin-2-yl]amino}pyridin-4-yl)methyl](methyl)oxido-lambda⁶-sulfanylidene]amino}-3-methyl-1-oxobutan-2-yl]carbamoyl}pyrrolidin-1-yl]carbonyl}-2,2-dimethyl-4,17-dioxo-3,8,11,14-tetraoxa-5,18-diazahenicosan-21-oate (Intermediate 7)

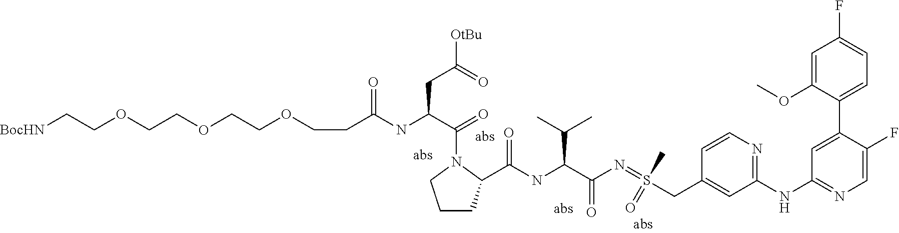 [SMILES: COc1cc(F)ccc1-c1cc(Nc2cc(C[S@](C)(=O)=NC(=O)[C@@H](NC(=O)[C@@H]3CCCN3C(=O)[C@H](CC(=O)OC(C)(C)C)NC(=O)CCOCCOCCOCCNC(=O)OC(C)(C)C)C(C)C)ccn2)ncc1F]

Trifluoroacetic acid-N—[(S)-[(2-{[5-fluoro-4-(4-fluoro-2-methoxyphenyl)pyridin-2-yl]amino} pyridin-4-yl)methyl](methyl)oxo-lambda⁶-sulfanylidene]-L-valinamide (1/1) (Intermediate 6) (26 mg, 42 μmol) and (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (Building block 3) 24.6 mg, 41.7 μmol) were dissolved in DMF (8 mL) and 1.3 eq EDCI (10.4 mg, 54.3 μmol), 1.5 eq HOBT hydrate (9.6 mg, 62.6 μmol). 3.0 eq N,N-diisopropylethylamine (22 μl, 125 μmol) were added. After stirring overnight at rt, the mixture was concentrated in vacuo and the residue was purified by prep. HPLC, concentrated, and lyophilized from ACN/H₂O to yield Intermediate 7 (44.0 mg, 93% purity, 91%). LC-MS: Rₜ=1.04 min; MS (ESIpos): m/z=1075 [M+H]⁺.

Example I8: Preparation of N-(3-{2-[2-(2-aminoethoxy)ethoxy]ethoxy}propanoyl)-L-alpha-aspartyl-L-prolyl-N—[(S*)-[(2-{[5-fluoro-4-(4-fluoro-2-methoxyphenyl)pyridin-2-yl]amino}pyridin-4-yl)methyl](methyl)oxido-lambda⁶-sulfanylidene]-L-valinamide trifluoroacetate (1:1) (Intermediate 8)

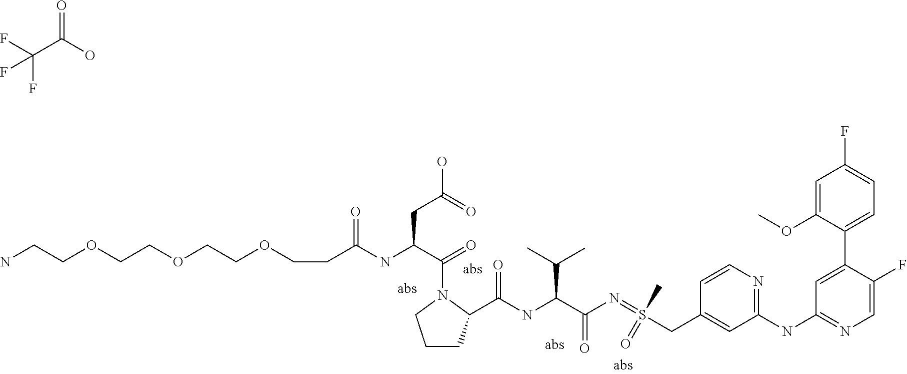 [SMILES: O=C(O)C(F)(F)F ; COc1cc(F)ccc1-c1cc(Nc2cc(C[S@](C)(=O)=NC(=O)[C@@H](NC(=O)[C@@H]3CCCN3C(=O)[C@H](CC(=O)O)NC(=O)CCOCCOCCOCCN)C(C)C)ccn2)ncc1F]

Tert-butyl (19S)-19-{[(2S)-2-{[(2S)-1-{[(S*)-[(2-{[5-fluoro-4-(4-fluoro-2-methoxyphenyl)pyridin-2-yl]amino}pyridin-4-yl)methyl](methyl)oxido-lambda⁶-sulfanylidene]amino}-3-methyl-1-oxobutan-2-yl]carbamoyl}pyrrolidin-1-yl]carbonyl}-2,2-dimethyl-4,17-dioxo-3,8,11,14-tetraoxa-5,18-diazahenicosan-21-oate (Intermediate 7) (44.0 mg, 38.0 μmol) was dissolved in DCM (5 mL). TFA (1.5 mL) was added, and the reaction mixture was stirred for 1 h at rt. It was concentrated in vacuo, the residue was dissolved in ACN/H₂O and lyophilized to give Intermediate 18 (41.0 mg, 90% purity, 94%). LC-MS: Rₜ=0.65 min; MS (ESIpos): m/z=919 [M+H]⁺.

Example I9: Preparation of tert-butyl [(2S)-1-{[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]amino}-3-methyl-1-oxobutan-2-yl]carbamate (Intermediate 9)

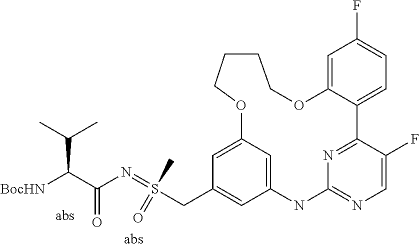 [SMILES: CC(C)[C@H](NC(=O)OC(C)(C)C)C(=O)N=[S@@](C)(=O)Cc1cc2cc(c1)OCCCCOc1cc(F)ccc1-c1nc(ncc1F)N2]

(S)-5-Fluoro-4-(4-fluoro-2-methoxyphenyl)-N-{4-[(S-methylsulfonimidoyl)methyl]pyridin-2-yl}pyridin-2-amine (Building block 6 (PT-3′)) (75.0 mg, 163 μmol) and N-(tert-butoxycarbonyl)-L-valine (42.5 mg, 195 μmol) were dissolved in DMF (7.5 mL) and 1.5 eq HATU (93 mg, 244 μmol). 3.0 eq N,N-diisopropylethylamine (85 μl, 489 μmol) were added. After stirring overnight at rt, the mixture was concentrated in vacuo and the residue was purified by prep. HPLC and concentrated to yield Intermediate 9 as a light-yellow foam. (90 mg, 100% purity, 84%). LC-MS: Rₜ=4.02 min; MS (ESIpos): m/z=660 [M+H]⁺.

Example I10: Preparation of N—[(S)-{[16,20-difluor-2,3,4,5-tetrahydro-12H-13,17-(azeno)-11,7-(metheno)-1,6,12,14-benzodioxadiazacyclononadecin-9-yl]methyl}(methyl)oxido-lambda⁶-sulfanyliden]-L-valinamidtrifluoracetat (1:1) (Intermediate 10)

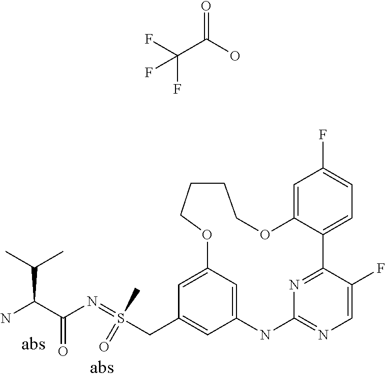 [SMILES: O=C(O)C(F)(F)F ; CC(C)[C@H](N)C(=O)N=[S@@](C)(=O)Cc1cc2cc(c1)OCCCCOc1cc(F)ccc1-c1nc(ncc1F)N2]

Tert-butyl [(2S)-1-{[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]amino}-3-methyl-1-oxobutan-2-yl]carbamate (Intermediate 9) (90 mg, 136 μmol) was dissolved in DCM (10 mL). TFA (2 mL) was added, and the reaction mixture was stirred for 1 h at rt. It was concentrated in vacuo, redissolved in ACN/H₂O and lyophilized to yield a light-yellow foam as Intermediate 10 in quantitative yield (96.0 mg, 100% purity, 100%). LC-MS: Rt=2.35 min; MS (ESIpos): m/z=560 [M+H]⁺.

Example I11: Preparation of tert-butyl-(19S)-19-{[(2S)-2-{[(2S)-1-{[(S)-{[5,23-difluor-8,13-dioxa-19,21,24-triazatetracyclo[18.3.1.1^{14,18}.0^{2,7}]pentacosa-1(24),2,4,6,14(25),15,17,20,22-nonaen-16-yl]methyl}(methyl) oxido-lambda⁶-sulfanyliden]amino}-3-methyl-1-oxobutan-2-yl]carbamoyl}pyrrolidin-1-yl] carbonyl}-2,2-dimethyl-4,17-dioxo-3,8,11,14-tetraoxa-5,18-diazahenicosan-21-oate (Intermediate 11)

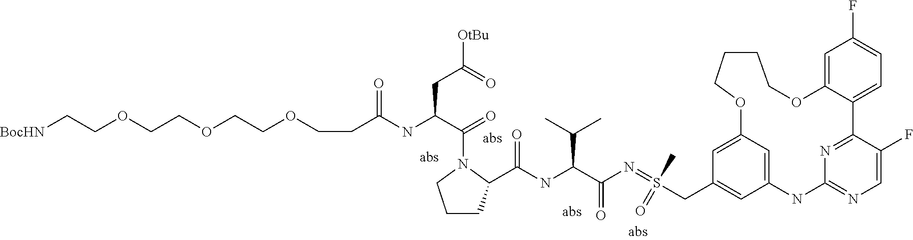 [SMILES: CC(C)[C@H](NC(=O)[C@@H]1CCCN1C(=O)[C@H](CC(=O)OC(C)(C)C)NC(=O)CCOCCOCCOCCNC(=O)OC(C)(C)C)C(=O)N=[S@@](C)(=O)Cc1cc2cc(c1)OCCCCOc1cc(F)ccc1-c1nc(ncc1F)N2]

N—[(S)-{[16,20-Difluor-2,3,4,5-tetrahydro-12H-13,17-(azeno)-11,7-(metheno)-1,6,12,14-benzodioxadiazacyclononadecin-9-yl]methyl}(methyl)oxido-lambda⁶-sulfanyliden]-L-valinamidtrifluoracetat (1:1) (Intermediate 10) (68 mg, 101 μmol) and (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid Building block 3 (71.4 mg, 121 μmol) were dissolved in DMF (8 mL). 1.3 eq HATU (57.6 mg, 151.4 μmol) and 3 eq N,N-diisopropylethylamine (53 μl, 303 μmol) were added. After stirring for 1 h at rt, the mixture was concentrated in vacuo and the residue was purified by prep. HPLC. Relevant fractions were collected and then evaporated to yield Intermediate 11 as a light-yellow foam (82.0 mg, 94% purity, 67.5%). LC-MS: Rₜ=4.1 min; MS (ESIpos): m/z=1131 [M+H]⁺.

Example I12: Preparation of N-(3-{2-[2-(2-aminoethoxy)ethoxy]ethoxy}propanoyl)-L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide trifluoroacetic acid (1/1) (Intermediate 12)

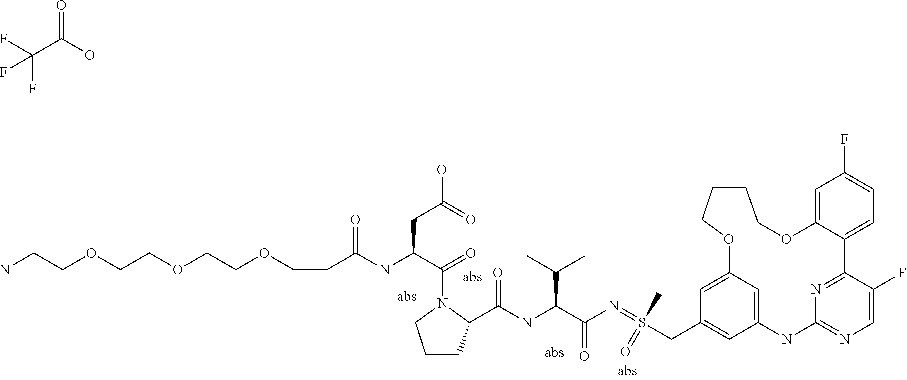 [SMILES: O=C(O)C(F)(F)F ; CC(C)[C@H](NC(=O)[C@@H]1CCCN1C(=O)[C@H](CC(=O)O)NC(=O)CCOCCOCCOCCN)C(=O)N=[S@@](C)(=O)Cc1cc2cc(c1)OCCCCOc1cc(F)ccc1-c1nc(ncc1F)N2]

Intermediate 11 (82 mg, 72.5 μmol) was dissolved in DCM (2 mL). TFA (5 mL) was added and the reaction mixture was stirred for 1 h at rt. It was concentrated in vacuo, redissolved in ACN/H₂O and lyophilized to yield a colorless foam as Intermediate 12 (78.0 mg, 91% purity, 90%). LC-MS: Rₜ=1.4 min; MS (ESIneg): m/z=973 [M−H].

Example I13: Preparation of N-{2-[{2-[(tert-butoxycarbonyl)(methyl)amino]ethyl}(methyl)amino]ethyl}-N-methylglycyl-L-asparaginyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (Intermediate 13)

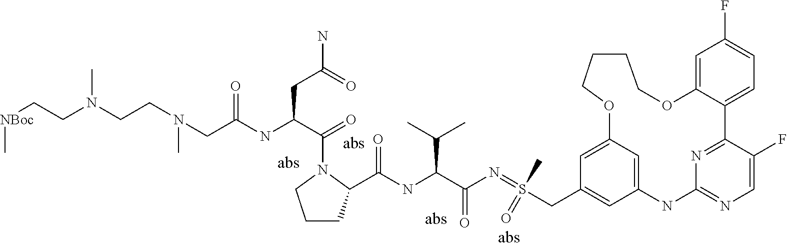 [SMILES: CC(C)[C@H](NC(=O)[C@@H]1CCCN1C(=O)[C@H](CC(N)=O)NC(=O)CN(C)CCN(C)CCN(C)C(=O)OC(C)(C)C)C(=O)N=[S@@](C)(=O)Cc1cc2cc(c1)OCCCCOc1cc(F)ccc1-c1nc(ncc1F)N2]

N—[(S)-{[16,20-Difluor-2,3,4,5-tetrahydro-12H-13,17-(azeno)-11,7-(metheno)-1,6,12,14-benzodioxadiazacyclononadecin-9-yl]methyl}(methyl)oxido-lambda⁶-sulfanyliden]-L-valinamidtrifluoracetat (1:1) (Intermediate 10) (50.0 mg, 74.2 μmol) was dissolved in DMF (3.4 mL) and butoxycarbonyl)(methyl)amino]ethyl}(methyl)amino]ethyl}-N-methylglycyl-L-asparaginyl-L-proline (Building block 9) (38.2 mg, 74.2 μmol), 1.5 eq HATU (42.3 mg, 111 μmol) and 3 eq N,N-diisopropylethylamine (39 μl, 220 μmol) were added. After stirring for 1 h at rt, the mixture was evaporated to dryness and the residue was purified by prep. HPLC. Relevant fractions were collected and then evaporated to yield Intermediate 13 (42 mg, 96% purity, 51%) as a light-yellow foam. LC-MS: Rₜ=2.74 min; MS (ESIpos): m/z=1055 [M+H]⁺.

Example I14: Preparation of trifluoroacetic acid N-methyl-N-(2-{methyl[2-(methylamino)ethyl]amino}ethyl)glycyl-L-asparaginyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (1/1) (Intermediate 14)

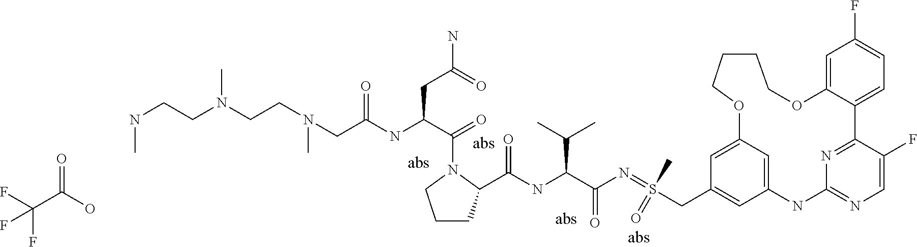 [SMILES: O=C(O)C(F)(F)F ; CNCCN(C)CCN(C)CC(=O)N[C@@H](CC(N)=O)C(=O)N1CCC[C@H]1C(=O)N[C@H](C(=O)N=[S@@](C)(=O)Cc1cc2cc(c1)OCCCCOc1cc(F)ccc1-c1nc(ncc1F)N2)C(C)C]

N-{2-[{2-[(Tert-butoxycarbonyl)(methyl)amino]ethyl}(methyl)amino]ethyl}-N-methylglycyl-L-asparaginyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (Intermediate 13) (42.0 mg, 39.8 μmol) was dissolved in DCM (5 mL). TFA (1 mL) was added, and the reaction mixture was stirred for 30 min at rt. It was concentrated in vacuo, the residue was dissolved in ACN/H₂O and lyophilized to give Intermediate 14) (40.0 mg, 96% purity, 90%) as a colorless foam. LC-MS: Rₜ=2.85 min; MS (ESIpos): m/z=956 [M+H]⁺.

Example I15: Preparation of trifluoroacetic acid N-methyl-N-(2-{methyl[2-(methylamino)ethyl]amino}ethyl)glycyl-L-asparaginyl-L-prolyl-N—[(S)-[(2-{[5-fluoro-4-(4-fluoro-2-methoxyphenyl)pyridin-2-yl]amino}pyridin-4-yl)methyl](methyl)oxo-lambda⁶-sulfanylidene]-L-valinamide (1/1) (Intermediate 15)

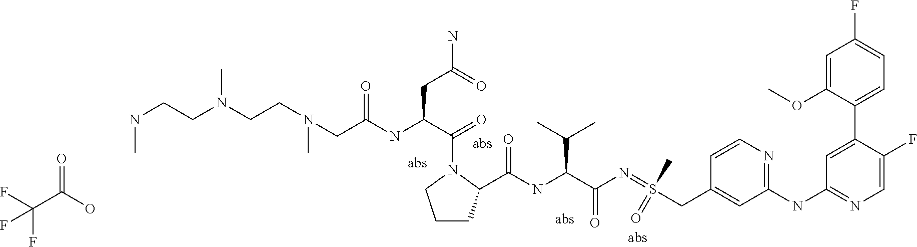 [SMILES: O=C(O)C(F)(F)F ; CNCCN(C)CCN(C)CC(=O)N[C@@H](CC(N)=O)C(=O)N1CCC[C@H]1C(=O)N[C@H](C(=O)N=[S@@](C)(=O)Cc1ccnc(Nc2cc(-c3ccc(F)cc3OC)c(F)cn2)c1)C(C)C]

Intermediate 15 synthesized in analogy to Intermediate 14 by coupling of Intermediate 6 with Building block 9 and subsequent deprotection with TFA. Yield: 46.0 mg (88% purity, 43% over 2 steps). LC-MS: Rₜ=2.35 min; MS (ESIpos): m/z=900 [M+H]⁺.

Example I16: Preparation of tert-butyl [(2S)-5-(carbamoylamino)-1-{[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]amino}-1-oxopentan-2-yl]carbamate (Intermediate 16)

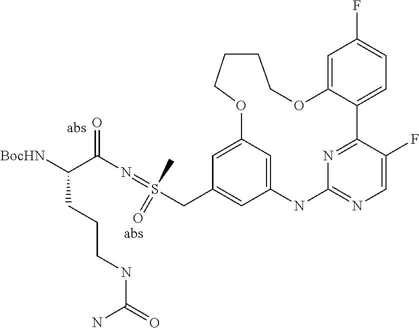 [SMILES: CC(C)(C)OC(=O)N[C@@H](CCCNC(N)=O)C(=O)N=[S@@](C)(=O)Cc1cc2cc(c1)OCCCCOc1cc(F)ccc1-c1nc(ncc1F)N2]

16,20-Difluoro-9-[(S-methylsulfonimidoyl)methyl]-2,3,4,5-tetrahydro-12H-13,17-(azeno)-11,7-(metheno)-1,6,12,14-benzodioxadiazacyclononadecine (Building block 6 (PT-3′) (30.0 mg, 65 μmol) and N²-(tert-butoxycarbonyl)-N⁵-carbamoyl-L-ornithine (35.9 mg, 130 μmol) were dissolved in DMF (10 mL) and 2.2 eq EDCI (27.5 mg, 143 μmol), 2.5 eq HOBT hydrate (24.9 mg, 163 μmol) as well as 4.0 eq N,N-diisopropylethylamine (45 μl, 260 μmol) were added. After stirring overnight at rt, 20 mg of each, N²-(tert-butoxycarbonyl)-N⁵-carbamoyl-L-ornithine, HOBT, and EDCI as well as 15 μl N,N-diisopropylethylamine were added and the mixture was stirred for another 3 h at rt. Subsequently it was concentrated in vacuo and the residue was purified by prep. HPLC. Relevant fractions were collected and concentrated to dryness to give Intermediate 16 (43 mg, 97% purity, 89%). LC-MS: Rₜ=1.84 min; MS (ESIpos): m/z=718 [M+H]⁺.

Example I17: Preparation of Trifluoroacetic acid N⁵-carbamoyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-ornithinamide (1/1) (Intermediate 17)

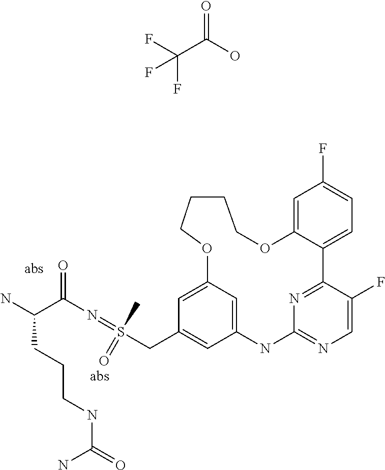 [SMILES: O=C(O)C(F)(F)F ; C[S@](=O)(Cc1cc2cc(c1)OCCCCOc1cc(F)ccc1-c1nc(ncc1F)N2)=NC(=O)[C@@H](N)CCCNC(N)=O]

Tert-butyl [(2S)-5-(carbamoylamino)-1-{[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]amino}-1-oxopentan-2-yl]carbamate (Intermediate 16) (50.0 mg, 97% purity, 67.4 μmol) was dissolved in DCM (10 mL), TFA (1.5 mL) was added and the reaction mixture was stirred for 30 min at rt. The residue was concentrated in vacuo, redissolved in ACN/H₂O and lyophilized to yield a yellow foam as Intermediate 17 (50.0 mg, 98% purity, 99%). LC-MS: Rₜ=1.26 min; MS (ESIpos): m/z=618 [M+H]⁺.

Example I18: Preparation of N-(tert-butoxycarbonyl)-L-valyl-N⁵-carbamoyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl} (methyl)oxo-lambda⁶-sulfanylidene]-L-ornithinamide (Intermediate 18)

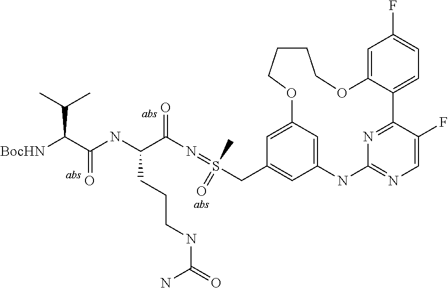 [SMILES: CC(C)[C@H](NC(=O)OC(C)(C)C)C(=O)N[C@@H](CCCNC(N)=O)C(=O)N=[S@@](C)(=O)Cc1cc2cc(c1)OCCCCOc1cc(F)ccc1-c1nc(ncc1F)N2]

Trifluoroacetic acid N⁵-carbamoyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-ornithinamide (1/1) (Intermediate 17) (50.0 mg, 98% purity, 66.8 μmol) and 2,5-dioxopyrrolidin-1-yl N-(tert-butoxycarbonyl)-L-valinate (27.3 mg, 86.9 μmol) were dissolved in DMF (10 mL) and 3 eq N,N-diisopropylethylamine (35 μl, 200 μmol) were added. After stirring overnight at rt, the mixture was concentrated in vacuo and the residue was purified by prep. HPLC. Relevant fractions were collected and then evaporated to dryness. The residue was dissolved in ACN/H₂O and lyophilized to obtain Intermediate 18 as a yellow foam (44.0 mg, 100% purity, 81%). LC-MS: Rₜ=2.06 min; MS (ESIpos): m/z=817 [M+H]⁺.

Example I19: Preparation of Trifluoroacetic acid L-valyl-N⁵-carbamoyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-ornithinamide (1/1) (Intermediate 19)

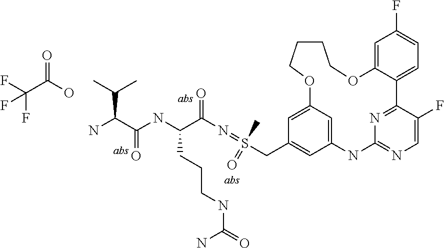

Intermediate 18 (44.0 mg, 53.9 μmol) was dissolved in DCM (10 mL), TFA (1.5 mL) was added, and the reaction mixture was stirred for 45 min at rt. It was concentrated in vacuo, dissolved in ACN/H₂O and lyophilized to yield a colorless foam as Intermediate 19 (44.0 mg, 100% purity, 98%). LC-MS: Rₜ=1.35 min; MS (ESIpos): m/z=717 [M+H]⁺.

Example I20: Preparation of N-(2,2-dimethyl-4,17-dioxo-3,8,11,14-tetraoxa-5-azaheptadecan-17-yl)-L-valyl-N⁵-carbamoyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-ornithinamide (Intermediate 20)

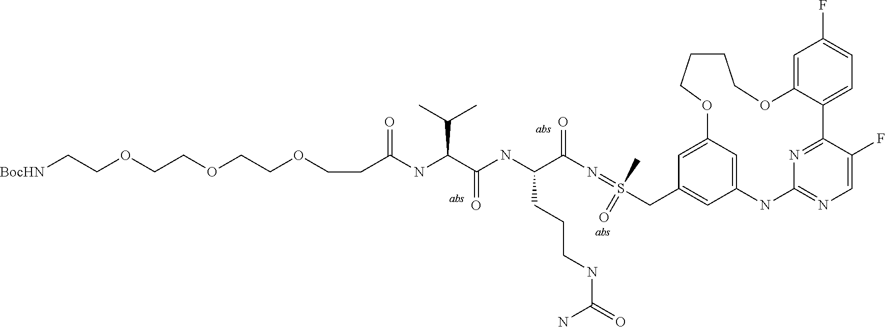 [SMILES: CC(C)[C@H](NC(=O)CCOCCOCCOCCNC(=O)OC(C)(C)C)C(=O)N[C@@H](CCCNC(N)=O)C(=O)N=[S@@](C)(=O)Cc1cc2cc(c1)OCCCCOc1cc(F)ccc1-c1nc(ncc1F)N2]

Trifluoroacetic acid L-valyl-N⁵-carbamoyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-ornithinamide (1/1) (Intermediate 19) (44.0 mg, 53.0 μmol) and tert-butyl {2-[2-(2-{3-[(2,5-dioxopyrrolidin-1-yl)oxy]-3-oxopropoxy}ethoxy)ethoxy]ethyl}carbamate (39.9 mg, 95.3 μmol) were dissolved in DMF (10 mL) and 4 eq N,N-diisopropylethylamine (37 μl, 210 μmol) were added. After stirring for 4.5 h at rt, the mixture was concentrated in vacuo and the residue was purified by prep. HPLC. Relevant fractions were collected and then evaporated to yield Intermediate 20 as a yellow foam (44.0 mg, 100% purity, 81%). LC-MS: Rₜ=1.93 min; MS (ESIpos): m/z=1020 [M+H]⁺.

Example I21: Preparation of Trifluoroacetic acid N-(3-{2-[2-(2-aminoethoxy)ethoxy]ethoxy}propanoyl)-L-valyl-N⁵-carbamoyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-ornithinamide (1/1) (Intermediate 21)

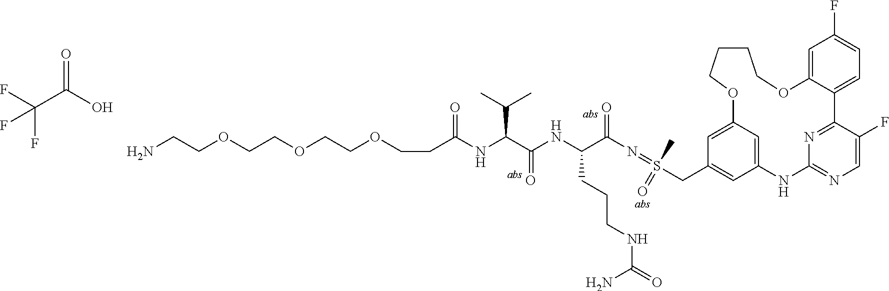 [SMILES: O=C(O)C(F)(F)F ; CC(C)[C@H](NC(=O)CCOCCOCCOCCN)C(=O)N[C@@H](CCCNC(N)=O)C(=O)N=[S@@](C)(=O)Cc1cc2cc(c1)OCCCCOc1cc(F)ccc1-c1nc(ncc1F)N2]

Intermediate 20 (44 mg, 43.1 μmol) was dissolved in DCM (10 mL). TFA (2 mL) was added, and the reaction mixture was stirred for 30 min at rt. It was concentrated in vacuo. The residue was dissolved in ACN/H₂O and lyophilized to yield Intermediate 21 as a yellow foam (44.0 mg, 100% purity, 99%). LC-MS: Rₜ=1.34 min; MS (ESIneg): m/z=918 [M−H].

Example I22: Preparation of tert-butyl {(2S)-1-{[(S)-{[5,23-difluoro-8,13-dioxa-19,21,24-triazatetracyclo[18.3.1.1^{14,18}.0^{2,7}] pentacosa-1(24),2,4,6,14(25),15,17,20,22-nonaen-16-yl]methyl} (methyl)oxo-lambda⁶-sulfanylidene]amino}-1,4-dioxo-4-[(triphenylmethyl) amino]butan-2-yl}carbamate (Intermediate 22)

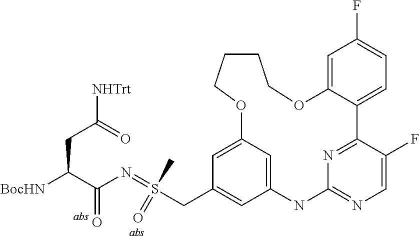

16,20-difluoro-9-[(S-methylsulfonimidoyl)methyl]-2,3,4,5-tetrahydro-12H-13,17-(azeno)-11,7-(metheno)-1,6,12,14-benzodioxadiazacyclononadecine (Building block 6 (PT-3′)) (50 mg 109 μmol) was dissolved in DMF (5 mL) and N²-(tert-butoxycarbonyl)-N-(triphenylmethyl)-L-asparagine (61.8 mg, 130 μmol) as well as 1.5 eq HATU (61.9 mg, 163 μmol) and 3 eq N,N-diisopropylethylamine (57 μl, 330 μmol) were added. After stirring for 1 h at rt, the mixture was evaporated to dryness and the residue was purified by prep. HPLC. Relevant fractions were collected and then evaporated to yield Intermediate 22 as a yellow foam in quantitative yield. (102 mg, 100%). LC-MS: Rₜ=4.82 min; MS (ESIpos): m/z=917 [M+H]⁺.

Example I23: Preparation of trifluoroacetic acid-N¹—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-aspartamide (1/1) (Intermediate 23)

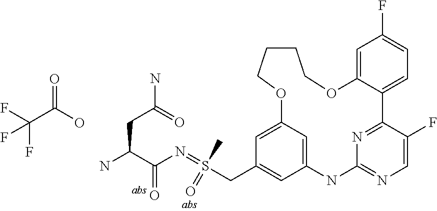 [SMILES: O=C(O)C(F)(F)F ; C[S@](=O)(Cc1cc2cc(c1)OCCCCOc1cc(F)ccc1-c1nc(ncc1F)N2)=NC(=O)[C@@H](N)CC(N)=O]

Tert-butyl {(2S)-1-{[(S)-{[5,23-difluoro-8,13-dioxa-19,21,24-triazatetracyclo[18.3.1.1^{14,18}.0^{2,7}] pentacosa-1(24),2,4,6,14(25),15,17,20,22-nonaen-16-yl]methyl} (methyl)oxo-lambda⁶-sulfanylidene]amino}-1,4-dioxo-4-[(triphenylmethyl) amino]butan-2-yl}carbamate (Intermediate 22) (101 mg, 110 μmol) was dissolved in TFA (10 mL) and the reaction mixture was stirred for 2 h at rt. It was concentrated in vacuo and the residue was purified by prep. HPLC. Relevant fractions were collected and then evaporated to yield Intermediate 23 as a colorless foam (60 mg, 100% purity, 79%). LC-MS: Rₜ=2.04 min; MS (ESIpos): m/z=575 [M+H]⁺.

Example I24: Preparation of N-(tert-butoxycarbonyl)-L-alanyl-N-methyl-L-alanyl-N¹—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-aspartamide (Intermediate 24)

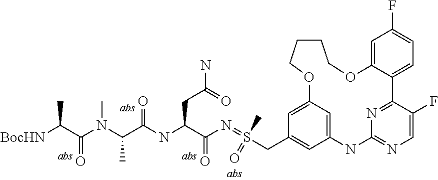 [SMILES: C[C@H](NC(=O)OC(C)(C)C)C(=O)N(C)[C@@H](C)C(=O)N[C@@H](CC(N)=O)C(=O)N=[S@@](C)(=O)Cc1cc2cc(c1)OCCCCOc1cc(F)ccc1-c1nc(ncc1F)N2]

Trifluoroacetic acid-N¹—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-aspartamide (1/1) (Intermediate 23) (60.0 mg, 87.1 μmol) and N-(tert-butoxycarbonyl)-L-alanyl-N-methyl-L-alanine (Building block 10) (28.7 mg, 105 μmol) were dissolved in DMF (10 mL) and 1.3 eq HATU (43.1 mg, 113 μmol) and 3 eq N,N-diisopropylethylamine (46 μl, 260 μmol) were added. After stirring for 30 min at rt, the mixture was evaporated to dryness and the residue was purified by prep. HPLC. Relevant fractions were collected and then evaporated to yield Intermediate 24 as a colorless foam (68 mg, 90% purity, 84%). LC-MS: Rₜ=3.26 min; MS (ESIpos): m/z=831 [M+H]⁺.

Example I25: Preparation of Trifluoroacetic acid-L-alanyl-N-methyl-L-alanyl-N¹—[(S)-{[16,20-difluoro-2,3,4,5-tetrahydro-12H-13,17-(azeno)-11,7-(metheno)-1,6,12,14-benzodioxadiazacyclononadecin-9-yl]methyl} (methyl)oxo-lambda⁶-sulfanylidene]-L-aspartamide (1/1) (Intermediate 25)

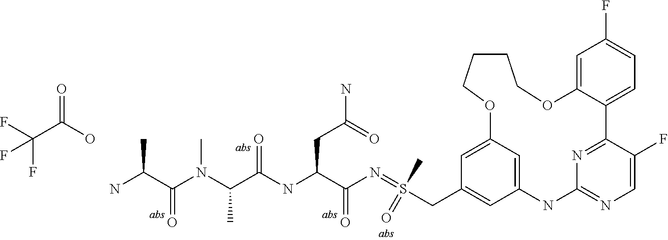 [SMILES: O=C(O)C(F)(F)F ; C[C@H](N)C(=O)N(C)[C@@H](C)C(=O)N[C@@H](CC(N)=O)C(=O)N=[S@@](C)(=O)Cc1cc2cc(c1)OCCCCOc1cc(F)ccc1-c1nc(ncc1F)N2]

Intermediate 24 (68.0 mg, 81.8 μmol) was dissolved in DCM (10 mL), TFA (2 mL) was added, and the reaction mixture was stirred for 30 min at rt. The residue was concentrated in vacuo, redissolved in ACN/H₂O and lyophilized to yield Intermediate 25 as a colorless foam (55.0 mg, 96% purity, 77%). LC-MS: Rₜ=1.29 min; MS (ESIpos): m/z=731 [M+H]⁺.

Example I26: Preparation of N-(2,2-dimethyl-4,17-dioxo-3,8,11,14-tetraoxa-5-azaheptadecan-17-yl)-L-alanyl-N-methyl-L-alanyl-N¹—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-aspartamide (Intermediate 26)

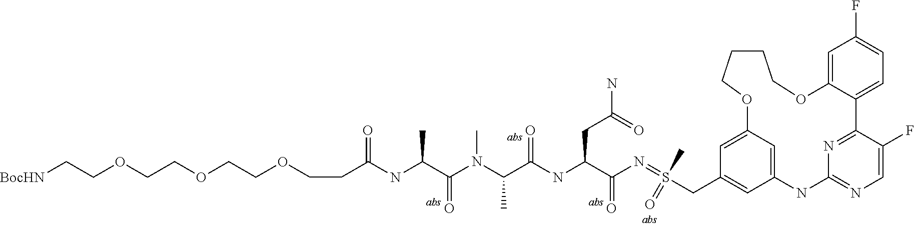 [SMILES: C[C@H](NC(=O)CCOCCOCCOCCNC(=O)OC(C)(C)C)C(=O)N(C)[C@@H](C)C(=O)N[C@@H](CC(N)=O)C(=O)N=[S@@](C)(=O)Cc1cc2cc(c1)OCCCCOc1cc(F)ccc1-c1nc(ncc1F)N2]

Intermediate 25 (55.0 mg, 65.1 μmol) and tert-butyl {2-[2-(2-{3-[(2,5-dioxopyrrolidin-1-yl)oxy]-3-oxopropoxy}ethoxy)ethoxy]ethyl}carbamate (32.7 mg, 78.1 μmol) were dissolved in DMF (8 mL) and 3 eq N,N-diisopropylethylamine (34 μl, 200 μmol) were added. After stirring for 3 h at rt, the mixture was in vacuo and the residue was purified by prep. HPLC. Relevant fractions were collected and then evaporated to yield Intermediate 26 as a colorless foam (61.0 mg, 93% purity, 84%). LC-MS: Rₜ=1.79 min; MS (ESIpos): m/z=1034 [M+H]⁺.

Example I27: Preparation of trifluoroacetic acid-N-(3-{2-[2-(2-aminoethoxy)ethoxy]ethoxy}propanoyl)-L-alanyl-N-methyl-L-alanyl-N¹—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-aspartamide (1/1) (Intermediate 27)

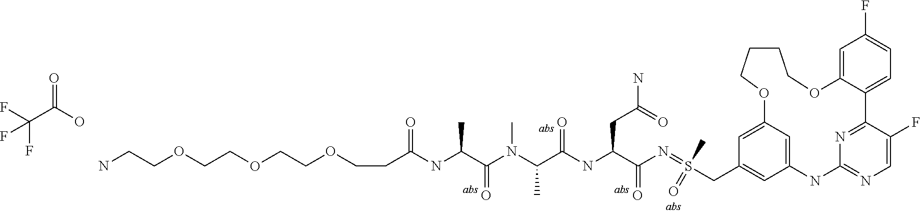 [SMILES: O=C(O)C(F)(F)F ; C[C@H](NC(=O)CCOCCOCCOCCN)C(=O)N(C)[C@@H](C)C(=O)N[C@@H](CC(N)=O)C(=O)N=[S@@](C)(=O)Cc1cc2cc(c1)OCCCCOc1cc(F)ccc1-c1nc(ncc1F)N2]

Intermediate 26 (61 mg, 59 μmol) was dissolved in DCM (7.2 mL). TFA (1.4 mL) was added, and the reaction mixture was stirred for 30 min at rt. It was subsequently concentrated in vacuo, the residue was dissolved in ACN/H₂O and lyophilized to yield Intermediate 27 as a colorless foam (47.0 mg, 100% purity, 76%). LC-MS: Rₜ=2.15 min; MS (ESIpos): m/z=934 [M+H]⁺.

Example I28: Preparation of tert-butyl [(2S)-1-{[(R or S*)—{[(4R or S*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda⁶-sulfanylidene]amino}-3-methyl-1-oxobutan-2-yl]carbamate (*single diastereoisomer) (Intermediate 28)

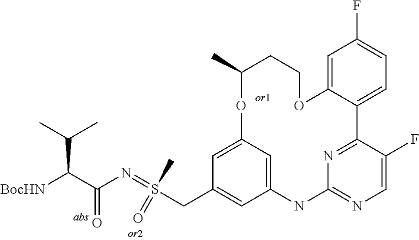 [SMILES: CC(C)[C@H](NC(=O)OC(C)(C)C)C(=O)N=[S@@](C)(=O)Cc1cc2cc(c1)O[C@@H](C)CCOc1cc(F)ccc1-c1nc(ncc1F)N2]

(4R or S*)-15,19-difluoro-8-[(R or S-methanesulfonimidoyl)methyl]-4-methyl-3,4-dihydro-2H,11H-12,16-(azeno)-10,6-(metheno)-1,5,11,13-benzodioxadiazacyclooctadecine (single diastereoisomer) Building block 7 (PT-4) (60.0 mg, 98% purity, 128 μmol) and N-(tert-butoxycarbonyl)-L-valine (41.7 mg, 192 μmol) were dissolved in DMF (12 mL) and 1.5 eq HATU (73 mg, 192 μmol) as well as 3.0 eq N,N-diisopropyl ethylamine (67 μl, 380 μmol) were added. After stirring over 3 days at rt, the mixture was concentrated in vacuo and the residue was purified by prep. HPLC the relevant fractions were collected and then concentrated. The residue was dissolved in ACN/H₂O and lyophilized to yield Intermediate 28 as a yellow foam (27 mg, 98% purity, 31%). LC-MS: Rₜ=2.41 min; MS (ESIpos): m/z=660 [M+H]⁺.

Example I29: Preparation of Trifluoroacetic acid-N—[(R or S*)—{[(4R or S*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda⁶-sulfanylidene]-L-valinamide (1/1) (*single diastereomer) (Intermediate 29)

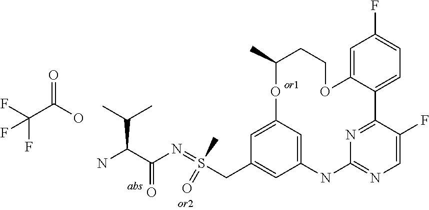

Intermediate 28 (27 mg, 40 μmol) was dissolved in DCM (8 mL). TFA (1 mL) was added and the reaction mixture was stirred for 30 min at rt. The residue was concentrated in vacuo, dissolved in ACN/H₂O and lyophilized to yield Intermediate 29 as a yellow foam (26.0 mg, 98% purity, 94%). LC-MS: Rₜ=1.56 min; MS (ESIpos): m/z=560 [M+H]⁺.

Example I30: Preparation of N-{2-[{2-[(tert-butoxycarbonyl)(methyl)amino]ethyl}(methyl)amino]ethyl}-N-methylglycyl-L-asparaginyl-L-prolyl-N—[(R or S*)—{[(4R or S*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda⁶-sulfanylidene]-L-valinamide (*single diastereomer) (Intermediate 30)

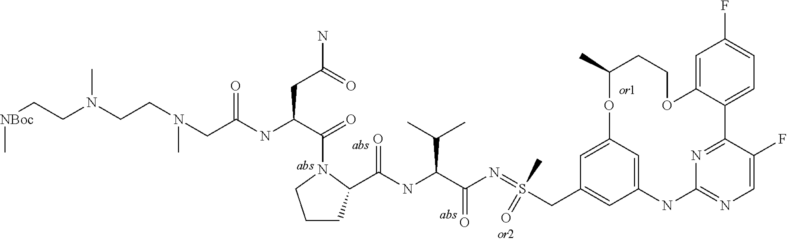 [SMILES: CC(C)[C@H](NC(=O)[C@@H]1CCCN1C(=O)[C@H](CC(N)=O)NC(=O)CN(C)CCN(C)CCN(C)C(=O)OC(C)(C)C)C(=O)N=[S@@](C)(=O)Cc1cc2cc(c1)O[C@@H](C)CCOc1cc(F)ccc1-c1nc(ncc1F)N2]

Intermediate 29 (26.0 mg, 37.7 μmol) was dissolved in DMF (6 mL) and butoxycarbonyl)(methyl)amino]ethyl}(methyl)amino]ethyl}-N-methylglycyl-L-asparaginyl-L-proline (Building block 9) (25.3 mg, 49.1 μmol), 1.5 eq HATU (21.5 mg, 56.6 μmol) and 4 eq N,N-diisopropylethylamine (26 μl, 150 μmol) were added. After stirring for 2 h at rt, the mixture was evaporated to dryness and the residue was purified by prep. HPLC. Relevant fractions were collected and concentrated in vacuo. The residue was dissolved in ACN/H₂O and lyophilized to yield Intermediate 30 as a light-yellow foam (31 mg, 100% purity, 78%). LC-MS: Rₜ=2.92 min; MS (ESIpos): m/z=1057 [M+H]⁺.

Example I31: Preparation of Trifluoroacetic acid-N-methyl-N-(2-{methyl[2-(methylamino)ethyl]amino}ethyl)glycyl-L-asparaginyl-L-prolyl-N—[(R or S*)—{[(4R or S*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda⁶-sulfanylidene]-L-valinamide (1/1) (*single diastereomer) (Intermediate 31)

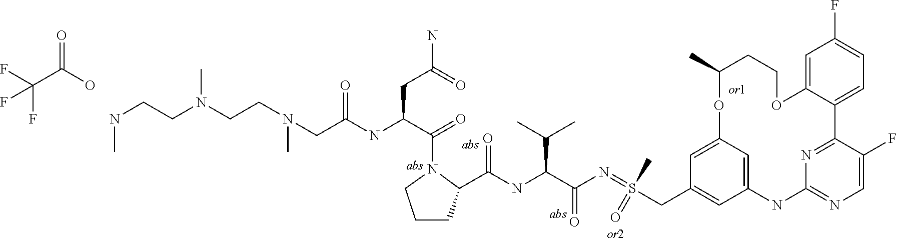

Intermediate 30 (31.0 mg, 29.3 μmol) was dissolved in DCM (6 mL). TFA (1 mL) was added, and the reaction mixture was stirred for 30 min at rt. It was concentrated in vacuo; the residue was dissolved in ACN/H₂O and lyophilized. 32 mg of Intermediate 31 was obtained as a yellow foam in quantitative yield (32.0 mg, 99% purity, 100%). LC-MS: Rₜ=1.14 min; MS (ESIpos): m/z=956 [M+H]⁺.

Example I32: Preparation of tert-butyl [(2S)-1-{[(R)-{[3,21-difluoro-13-oxa-5,7,19,26-tetraazatetracyclo[18.3.1.1^{2,6}.1^{8,12}] hexacosa-1(24),2(26),3,5,8(25),9,11,20,22-nonaen-10-yl]methyl}(methyl)oxo-lambda⁶-sulfanylidene]amino}-3-methyl-1-oxobutan-2-yl]carbamate (Intermediate 32-(R)) and of tert-butyl [(2S)-1-{[(S)-{[3,21-difluoro-13-oxa-5,7,19,26-tetraazatetracyclo[18.3.1.1^{2,6}.1^{8,12}] hexacosa-1(24),2(26),3,5,8(25),9,11,20,22-nonaen-10-yl]methyl}(methyl)oxo-lambda⁶-sulfanylidene]amino}-3-methyl-1-oxobutan-2-yl]carbamate (Intermediate 32-(S))

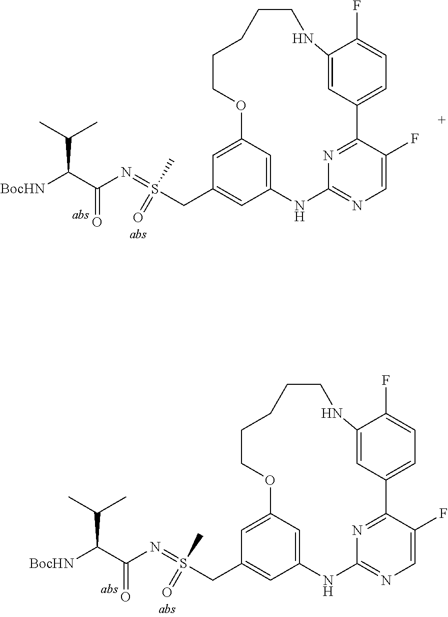 [SMILES: CC(C)[C@H](NC(=O)OC(C)(C)C)C(=O)N=[S@](C)(=O)Cc1cc2cc(c1)OCCCCCNc1cc(ccc1F)-c1nc(ncc1F)N2 ; CC(C)[C@H](NC(=O)OC(C)(C)C)C(=O)N=[S@@](C)(=O)Cc1cc2cc(c1)OCCCCCNc1cc(ccc1F)-c1nc(ncc1F)N2]

3,21-Difluoro-10-[(S-methanesulfonimidoyl)methyl]-13-oxa-5,7,19,26-tetraazatetracyclo[18.3.1.1^{2,6}.1^{8,12}]hexacosa-1(24),2(26),3,5,8(25),9,11,20,22-nonaene (Building block 13) (80.0 mg, 169 μmol) was dissolved in DMF (10 mL). N-(tert-butoxycarbonyl)-L-valine (44.0 mg, 203 μmol), HATU (96.4 mg, 253 μmol) and DIEA (88 μl, 510 μmol) were added. The reaction was stirred overnight at RT and then left over the weekend. The reaction was evaporated to dryness using a rotary evaporator. The residue was separated twice by prep. HPLC to give Intermediate 32-(S or R*) (38 mg, 33% yield) as a light-yellow foam and Intermediate 32-(R or S*) (48 mg, 39% yield). *Isolated as two separate diastereomers (S/S or S/R), absolute stereochemistry unknown.

Example I33: Preparation of Trifluoroacetic acid-N—[(S or R*)—{[3,21-difluoro-13-oxa-5,7,19,26-tetraazatetracyclo[18.3.1.1^{2,6}.1^{8,12}] hexacosa-1(24),2(26),3,5,8(25),9,11,20,22-nonaen-10-yl]methyl}(methyl)oxo-lambda⁶-sulfanylidene]-L-valinamide (1/1) (Intermediate 33)

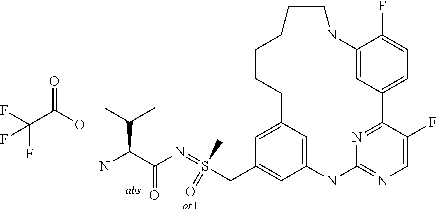

Tert-butyl [(2S)-1-{[(S or R*)—{[3,21-difluoro-13-oxa-5,7,19,26-tetraazatetracyclo[18.3.1.1^{2,6}.1^{8,12}] hexacosa-1(24),2(26),3,5,8(25),9,11,20,22-nonaen-10-yl]methyl}(methyl)oxo-lambda⁶-sulfanylidene]amino}-3-methyl-1-oxobutan-2-yl]carbamate (Intermediate 32-(S or R*)) (43.0 mg, 63.9 μmol) was dissolved in DCM (10 mL), then TFA (2.0 mL) was added. The reaction was stirred for 1 h at RT. The reaction was concentrated in vacuo. The residue was dissolved in ACN/water and freeze-dried to give Intermediate 33 (43.0 mg, 100% purity, 98% yield) as yellow foam. LC-MS: Rₜ=2.77 min; MS (ESIpos): m/z=573 [M+H]⁺.

Example I34: Preparation of N-methyl-N-(2,2,8,11-tetramethyl-4-oxo-3-oxa-5,8,11-triazatridecan-13-yl)glycyl-L-asparaginyl-L-prolyl-N—[(S or R*)—{[3,21-difluoro-13-oxa-5,7,19,26-tetraazatetracyclo[18.3.1.1^{2,6}.1^{8,12}]hexacosa-1(24),2(26),3,5,8(25),9,11,20,22-nonaen-10-yl]methyl}(methyl)oxo-lambda⁶-sulfanylidene]-L-valinamide (Intermediate 34)

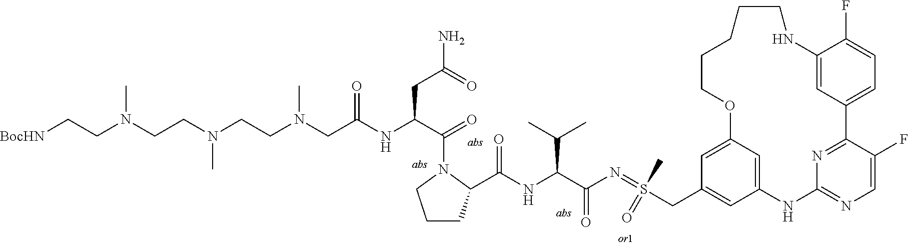 [SMILES: CC(C)[C@H](NC(=O)[C@@H]1CCCN1C(=O)[C@H](CC(N)=O)NC(=O)CN(C)CCN(C)CCN(C)CCNC(=O)OC(C)(C)C)C(=O)N=[S@@](C)(=O)Cc1cc2cc(c1)OCCCCCNc1cc(ccc1F)-c1nc(ncc1F)N2]

Trifluoroacetic acid-N—[(S or R*)—{[3,21-difluoro-13-oxa-5,7,19,26-tetraazatetracyclo[18.3.1.1^{2,6}.1^{8,12}] hexacosa-1(24),2(26),3,5,8(25),9,11,20,22-nonaen-10-yl]methyl}(methyl)oxo-lambda⁶-sulfanylidene]-L-valinamide (1/1) (Intermediate 33) (43.0 mg, 62.6 μmol) was dissolved in DMF (5 mL). N-methyl-N-(2,2,8,11-tetramethyl-4-oxo-3-oxa-5,8,11-triazatridecan-13-yl)glycyl-L-asparaginyl-L-proline (Building block 12) (34.9 mg, 62.6 μmol), HATU (35.7 mg, 93.9 μmol) and DIEA (22 μl, 130 μmol) were added. The reaction was stirred at RT for 1 h. The reaction was concentrated in vacuo. The residue was separated by prep. HPLC to give Intermediate 34 (32.0 mg, 100% purity, 46% yield) as light-yellow foam. LC-MS: Rₜ=2.56 min; MS (ESIpos): m/z=1113 [M+H]⁺.

Example I35: Preparation of trifluoroacetic acid-N-{2-[{2-[(2-aminoethyl)(methyl)amino]ethyl}(methyl)amino]ethyl}-N-methylglycyl-L-asparaginyl-L-prolyl-N—[(S or R*)—{[3,21-difluoro-13-oxa-5,7,19,26-tetraazatetracyclo[18.3.1.1^{2,6}.1^{8,12}]hexacosa-1(24),2(26),3,5,8(25),9,11,20,22-nonaen-10-yl]methyl}(methyl)oxo-lambda⁶-sulfanylidene]-L-valinamide (1/1) (Intermediate 35)

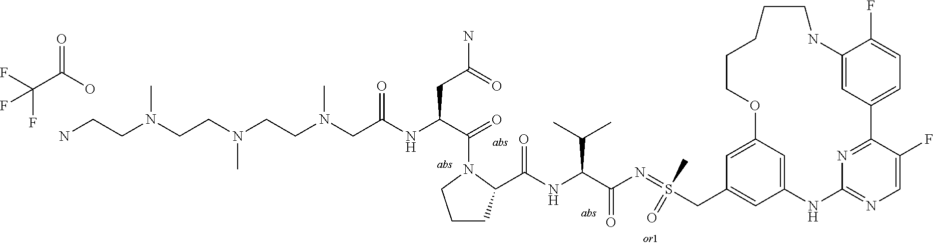

N-methyl-N-(2,2,8,11-tetramethyl-4-oxo-3-oxa-5,8,11-triazatridecan-13-yl)glycyl-L-asparaginyl-L-prolyl-N—[(S or R*)—{[3,21-difluoro-13-oxa-5,7,19,26-tetraazatetracyclo[18.3.1.1^{2,6}.1^{8,12}] hexacosa-1(24),2(26),3,5,8(25),9,11,20,22-nonaen-10-yl]methyl}(methyl) oxo-lambda⁶-sulfanylidene]-L-valinamide (Intermediate 34) (32.0 mg, 28.8 μmol) was dissolved in DCM (5.0 mL), then TFA (1.0 mL) was added. The reaction was stirred for 1 h at RT and then concentrated in vacuo. The residue was dissolved in ACN/water and freeze-dried to give Intermediate 35 (25.5 mg, 100% purity, 79% yield) as a yellow foam. LC-MS: Rₜ=2.92 min; MS (ESIpos): m/z=1013 [M+H]⁺. ¹H NMR (700 MHz, DMSO-d₆) δ ppm 0.853 (br t, J=8.21 Hz, 6H) 0.894 (br s, 1H) 1.575 (br s, 2H) 1.742-1.837 (m, 4H) 1.891 (br d, J=7.21 Hz, 2H) 1.939-2.022 (m, 1H) 2.131 (br d, J=5.62 Hz, 1H) 2.393-2.482 (m, 1H) 2.552 (br s, 3H) 2.581 (br s, 3H) 2.605-2.664 (m, 1H) 2.700 (br s, 2H) 2.734 (br s, 1H) 3.058 (br s, 6H) 3.086-3.164 (m, 4H) 3.218 (br s, 3H) 3.247 (br s, 3H) 3.618-3.657 (m, 1H) 3.680 (brs, 1H) 3.714-3.813 (m, 1H) 4.005 (br s, 2H) 4.125 (br d, J=5.93 Hz, 1H) 4.311 (br s, 1H) 4.495 (br d, J=7.95 Hz, 1H) 4.684-4.836 (m, 1H) 4.788 (br s, 2H) 4.950 (br s, 1H) 6.626 (br s, 1H) 6.817 (br s, 1H) 7.006 (br s, 1H) 7.161-7.202 (m, 1H) 7.220 (br s, 1H) 7.515 (br s, 1H) 7.556 (br d, J=8.27 Hz, 1H) 7.820 (br d, J=9.01 Hz, 1H) 8.112 (br s, 1H) 8.161 (br s, 1H) 8.366 (br s, 1H) 8.614 (br s, 1H) 8.872 (br s, 1H) 9.794 (br s, 1H).

Example I36: Preparation of Trifluoroacetic acid-N—[(R or S*)—{[3,21-difluoro-13-oxa-5,7,19,26-tetraazatetracyclo[18.3.1.1^{2,6}.1^{8,12}] hexacosa-1(24),2(26),3,5,8(25),9,11,20,22-nonaen-10-yl]methyl}(methyl)oxo-lambda⁶-sulfanylidene]-L-valinamide (1/1) (Intermediate 36)

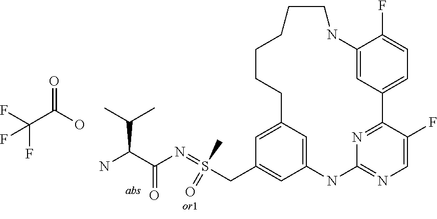

Tert-butyl [(2S)-1-{[(R or S*)—{[3,21-difluoro-13-oxa-5,7,19,26-tetraazatetracyclo[18.3.1.1^{2,6}.1^{8,12}] hexacosa-1(24),2(26),3,5,8(25),9,11,20,22-nonaen-10-yl]methyl}(methyl)oxo-lambda⁶-sulfanylidene] amino}-3-methyl-1-oxobutan-2-yl]carbamate (Intermediate 32-(R or S*)) (53.0 mg, 78.8 μmol) was dissolved in DCM (10 mL), then TFA (2.0 mL) was added. The reaction was stirred for 1 h at RT and then concentrated in vacuo. The residue was dissolved in ACN/water and lyophilized to give Intermediate 36 (47.0 mg, 100% purity, 87% yield) as a yellow foam. LC-MS: Rₜ=2.77 min; MS (ESIpos): m/z=573 [M+H]⁺.

Example I37: Preparation of N-methyl-N-(2,2,8,11-tetramethyl-4-oxo-3-oxa-5,8,11-triazatridecan-13-yl)glycyl-L-asparaginyl-L-prolyl-N—[(R or S*)—{[3,21-difluoro-13-oxa-5,7,19,26-tetraazatetracyclo[18.3.1.1^{2,6}.1^{8,12}]hexacosa-1(24),2(26),3,5,8(25),9,11,20,22-nonaen-10-yl]methyl}(methyl)oxo-lambda⁶-sulfanylidene]-L-valinamide (Intermediate 37)

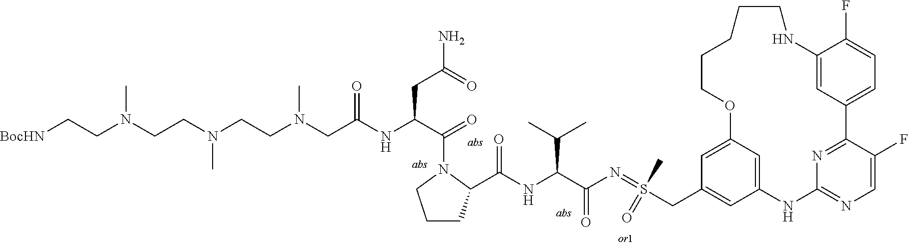 [SMILES: CC(C)[C@H](NC(=O)[C@@H]1CCCN1C(=O)[C@H](CC(N)=O)NC(=O)CN(C)CCN(C)CCN(C)CCNC(=O)OC(C)(C)C)C(=O)N=[S@@](C)(=O)Cc1cc2cc(c1)OCCCCCNc1cc(ccc1F)-c1nc(ncc1F)N2]

Trifluoroacetic acid-N—[(R or S*)—{[3,21-difluoro-13-oxa-5,7,19,26-tetraazatetracyclo[18.3.1.12,6.18,12] hexacosa-1(24),2(26),3,5,8(25),9,11,20,22-nonaen-10-yl]methyl}(methyl) oxo-lambda6-sulfanylidene]-L-valinamide (1/1) (Intermediate 36) (47.0 mg, 68.4 μmol) was dissolved in DMF (5.0 mL). N-methyl-N-(2,2,8,11-tetramethyl-4-oxo-3-oxa-5,8,11-triazatridecan-13-yl)glycyl-L-asparaginyl-L-proline (Building block 12) (38.2 mg, 68.4 μmol), HATU (39.0 mg, 103 μmol) and DIEA (24 μl, 140 μmol were added. The reaction was stirred at RT for 1 h, then it was concentrated in vacuo. The residue was separated by prep. HPLC to give Intermediate 37 (46.0 mg, 78% purity, 47% yield) as a yellow oil. LC-MS: Rₜ=2.58 min; MS (ESIpos): m/z=1113 [M+H]⁺.

Example I38: Preparation of trifluoroacetic acid-N-{2-[{2-[(2-aminoethyl)(methyl)amino]ethyl}(methyl)amino]ethyl}-N-methylglycyl-L-asparaginyl-L-prolyl-N—[(R or S*)—{[3,21-difluoro-13-oxa-5,7,19,26-tetraazatetracyclo[18.3.1.1^{2,6}.1^{8,12}]hexacosa-1(24),2(26),3,5,8(25),9,11,20,22-nonaen-10-yl]methyl}(methyl)oxo-lambda⁶-sulfanylidene]-L-valinamide (1/1) (Intermediate 38)

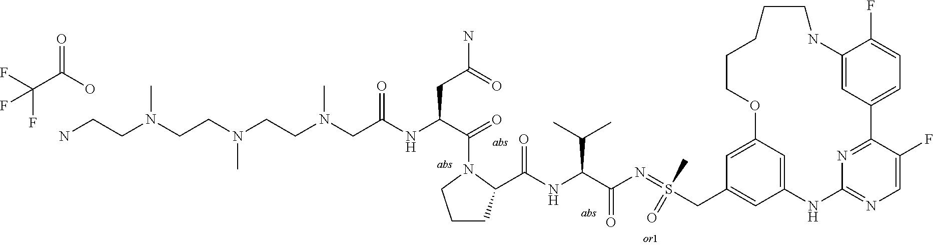

N-methyl-N-(2,2,8,11-tetramethyl-4-oxo-3-oxa-5,8,11-triazatridecan-13-yl)glycyl-L-asparaginyl-L-prolyl-N—[(R or S*)—{[3,21-difluoro-13-oxa-5,7,19,26-tetraazatetracyclo[18.3.1.1^{2,6}.1^{8,12}] hexacosa-1(24),2(26),3,5,8(25),9,11,20,22-nonaen-10-yl]methyl}(methyl) oxo-lambda⁶-sulfanylidene]-L-valinamide (Intermediate 37) (46.0 mg, 78% purity, 32.3 μmol) was dissolved in DCM (5 mL), then TFA (1.0 mL) was added. The reaction was stirred for 1 h at RT and concentrated in vacuo. The residue was separated by prep. HPLC to give Intermediate 38 (27.4 mg, 100% purity, 75% yield) as a yellow foam. LC-MS: Rₜ=2.93 min; MS (ESIpos): m/z=1013 [M+H]⁺. ¹H NMR (700 MHz, DMSO-d₆) δ ppm 0.823-0.908 (m, 6H) 1.577 (br s, 2H) 1.805 (br s, 4H) 1.886 (br s, 1H) 1.954 (br d, J=9.11 Hz, 2H) 1.982-2.066 (m, 1H) 2.161 (br dd, J=12.82, 6.25 Hz, 1H) 2.422-2.496 (m, 1H) 2.536-2.618 (m, 5H) 2.632-2.761 (m, 4H) 3.068 (br s, 5H) 3.124 (br s, 1H) 3.145 (br s, 5H) 3.180-3.272 (m, 4H) 3.572-3.708 (m, 1H) 4.006 (br d, J=18.54 Hz, 2H) 4.123 (br d, J=5.62 Hz, 1H) 4.544 (br d, J=8.05 Hz, 1H) 4.768 (br d, J=13.67 Hz, 1H) 4.935 (br d, J=13.67 Hz, 1H) 4.987 (br s, 1H) 6.696 (br s, 1H) 6.797 (br s, 1H) 7.023 (br s, 1H) 7.157-7.206 (m, 1H) 7.221 (br s, 1H) 7.523 (br s, 1H) 7.555 (br d, J=8.05 Hz, 1H) 7.883 (br d, J=8.69 Hz, 1H) 7.994-8.287 (m, 1H) 8.165 (br s, 1H) 8.368 (br s, 1H) 8.620 (br d, J=2.33 Hz, 1H) 8.892 (br s, 1H) 9.751 (s, 1H).

Example I39: Preparation of N-(3-{2-[2-(2-{[N²,N⁶-bis(tert-butoxycarbonyl)-L-lysyl]amino}ethoxy)ethoxy]ethoxy}propanoyl) -L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (Intermediate 39)

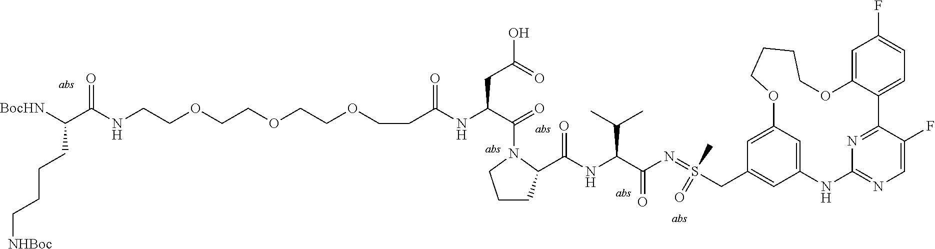 [SMILES: CC(C)[C@H](NC(=O)[C@@H]1CCCN1C(=O)[C@H](CC(=O)O)NC(=O)CCOCCOCCOCCNC(=O)[C@H](CCCCNC(=O)OC(C)(C)C)NC(=O)OC(C)(C)C)C(=O)N=[S@@](C)(=O)Cc1cc2cc(c1)OCCCCOc1cc(F)ccc1-c1nc(ncc1F)N2]

N-(3-{2-[2-(2-Aminoethoxy)ethoxy]ethoxy}propanoyl)-L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide trifluoroacetic acid (1/1) (Intermediate 12) (55.0 mg, 50.5 μmol) was dissolved in DMF (10 mL). 2,5-Dioxopyrrolidin-1-yl N²,N⁶-bis(tert-butoxycarbonyl)-L-lysinate (29.1 mg, 65.6 μmol) and DIEA (26 μl, 150 μmol) were added. The reaction was stirred at RT for 20 h and then concentrated in vacuo. The residue was separated by prep. HPLC to give Intermediate 39 (49.0 mg, 100% purity, 74% yield) as a yellow foam. LC-MS: Rₜ=3.77 min; MS (ESIpos): m/z=1304 [M+H]⁺.

Example I40: Preparation of N-[3-(2-{2-[2-(L-lysylamino)ethoxy]ethoxy}ethoxy)propanoyl]-L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide-trifluoroacetic acid (1/2) (Intermediate 40)

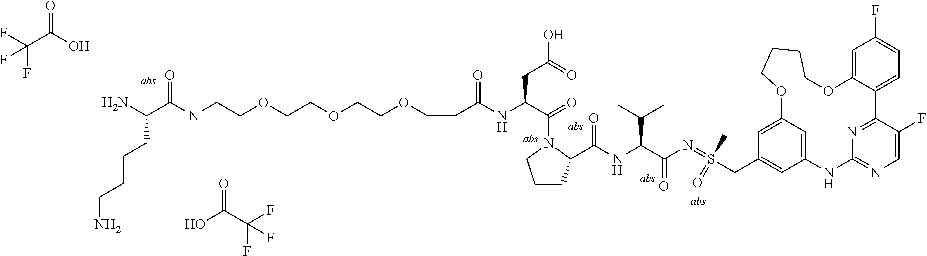 [SMILES: O=C(O)C(F)(F)F ; CC(C)[C@H](NC(=O)[C@@H]1CCCN1C(=O)[C@H](CC(=O)O)NC(=O)CCOCCOCCOCCNC(=O)[C@@H](N)CCCCN)C(=O)N=[S@@](C)(=O)Cc1cc2cc(c1)OCCCCOc1cc(F)ccc1-c1nc(ncc1F)N2 ; O=C(O)C(F)(F)F]

N-(3-{2-[2-(2-{[N²,N⁶-Bis(tert-butoxycarbonyl)-L-lysyl]amino}ethoxy) ethoxy]ethoxy} propanoyl) -L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (Intermediate 39) (49.0 mg, 37.6 μmol) was dissolved in DCM (10 mL), then TFA (2.0 mL) was added. The reaction was stirred for 1 h at RT and then concentrated in vacuo. The residue was dissolved in ACN/water and freeze-dried to give Intermediate 40 (49.0 mg, 100% purity, 98% yield) as a yellow foam. LC-MS: Rₜ=1.17 min; MS (ESIpos): m/z=1103 [M+H]⁺.

Example I41: Preparation of N-(3-{2-[2-(2-{[N²,N⁶-bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (Intermediate 41)

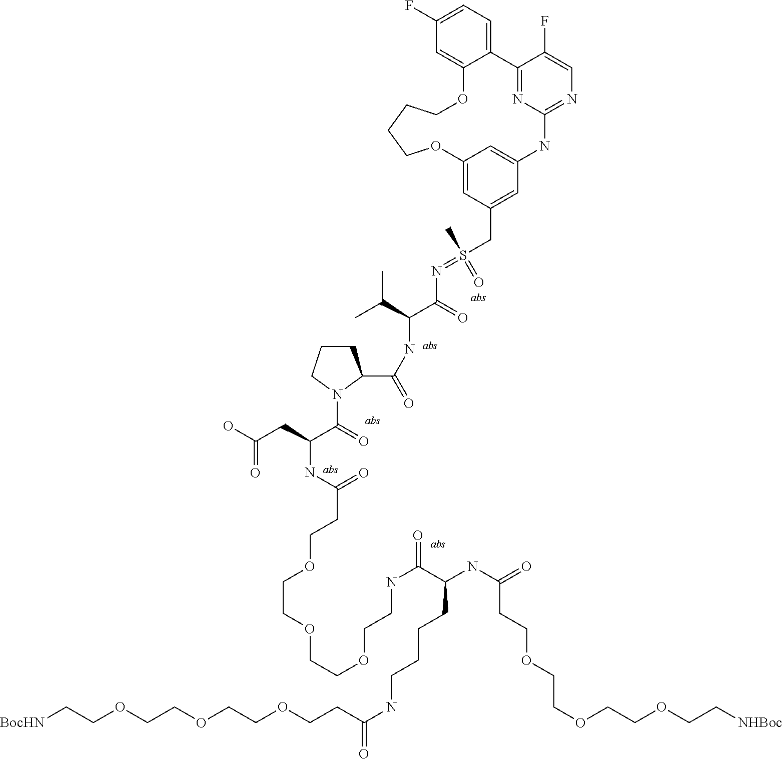

N-[3-(2-{2-[2-(L-Lysylamino)ethoxy]ethoxy}ethoxy)propanoyl]-L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide trifluoroacetic acid (1/2) (Intermediate 40) (49.0 mg, 36.8 μmol) was dissolved in DMF (10.0 mL). Tert-butyl {2-[2-(2-{3-[(2,5-dioxopyrrolidin-1-yl)oxy]-3-oxopropoxy}ethoxy) ethoxy]ethyl}carbamate (37.0 mg, 88.3 μmol) and DIEA (51 μl, 290 μmol) were added. The reaction was stirred at RT for 3 h and then concentrated in vacuo. The residue was purified by prep. HPLC to give Intermediate 41 (49.0 mg, 100% purity, 78% yield) as a colorless foam. LC-MS: Rₜ=4.71 min; MS (ESIpos): m/z=1709 [M+H]⁺.

Example I42: Preparation of N-(3-{2-[2-(2-{[N²,N⁶-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide-trifluoroacetic acid (1/2) (Intermediate 42)

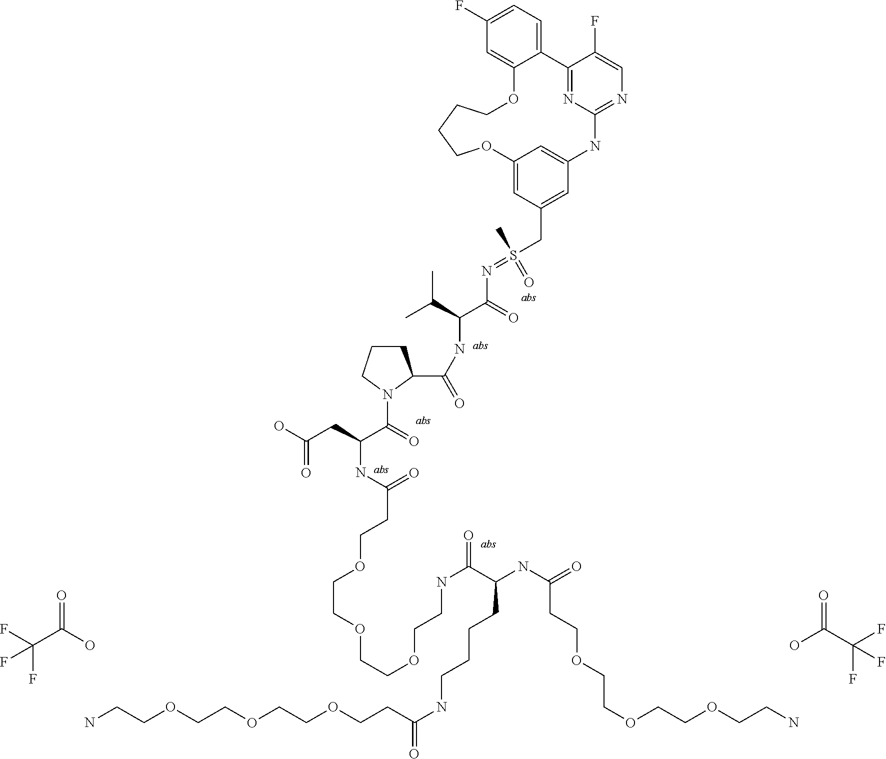

N-(3-{2-[2-(2-{[N²,N⁶-Bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiaza cyclononadecin-9-yl]methyl}(methyl)oxo-lambda⁶-sulfanylidene]-L-valinamide Intermediate 41 (49.0 mg, 28.7 μmol) was dissolved in DCM (7.6 mL), then TFA (1.5 mL) was added. The reaction was stirred for 1 h at RT and then concentrated in vacuo. The residue was dissolved in ACN/water and freeze-dried to give Intermediate 42 (49.0 mg, 100% purity, 98% yield) as a yellow foam. LC-MS: Rₜ=2.75 min; MS (ESIpos): m/z=1509 [M+H]⁺.

Example I43: Preparation of tert-butyl [(2S)-1-{[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]amino}-3-methyl-1-oxobutan-2-yl]carbamate (Intermediate 43)

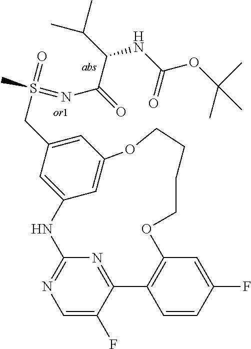 [SMILES: CC(C)[C@H](NC(=O)OC(C)(C)C)C(=O)N=[S@@](C)(=O)Cc1cc2cc(c1)OCCCCOc1cc(F)ccc1-c1nc(ncc1F)N2]

16,20-Difluoro-9-[(S-methanesulfonimidoyl)methyl]-2,3,4,5-tetrahydro-12H-13,17-(azeno)-11,7-(metheno)-1,6,12,14-benzodioxadiazacyclononadecine (300 mg, 651 μmol) (Building block 15) was dissolved in DMF (40 mL). N-(tert-butoxycarbonyl)-L-valine (170 mg, 782 μmol), HATU (372 mg, 977 μmol; CAS-RN:[148893-10-1]) and DIEA (340 μl, 2.0 mmol; CAS-RN:[7087-68-5]) were added. The reaction was stirred at RT for 24 hour and then evaporated to dryness using a rotary evaporator. The residue was separated by prep. HPLC to give Intermediate 43 (274 mg, 99% purity, 63% yield) as a light-yellow foam. LC-MS (Method 1): Rₜ=2.29 min; MS (ESIpos): m/z=660 [M+H]⁺.

Example I44: Preparation of trifluoroacetic acid N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (1/1) (Intermediate 44)

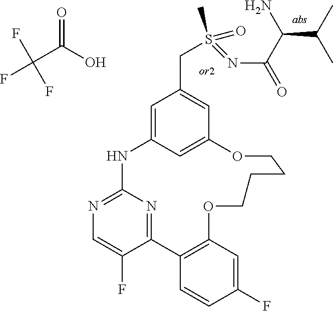 [SMILES: O=C(O)C(F)(F)F ; CC(C)[C@H](N)C(=O)N=[S@@](C)(=O)Cc1cc2cc(c1)OCCCCOc1cc(F)ccc1-c1nc(ncc1F)N2]

Tert-butyl [(2S)-1-{[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]amino}-3-methyl-1-oxobutan-2-yl]carbamate (274 mg, 415 μmol) (Example I43) was dissolved in DCM (30 ml), then TFA (5.0 ml, 65 mmol; CAS-RN:[76-05-1]) was added. The reaction was stirred for 1 hour at RT and then concentrated in vacuo. The residue was dissolved in ACN/water and freeze-dried to give Intermediate 44 (299 mg, 99% purity, 105% yield) as a light-yellow foam. LC-MS (Method 2): Rₜ=1.32 min; MS (ESIpos): m/z=560 [M+H]⁺.

Example I45: Preparation of tert-butyl (19S)-19-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 45)

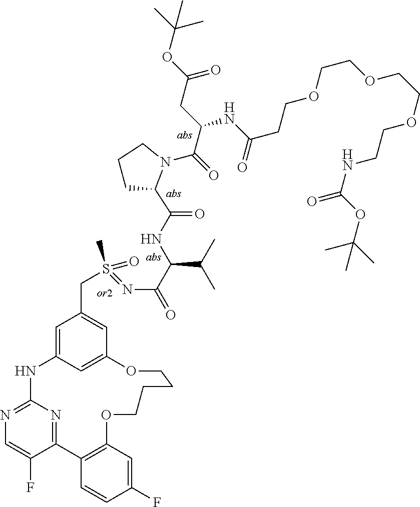 [SMILES: CC(C)[C@H](NC(=O)[C@@H]1CCCN1C(=O)[C@H](CC(=O)OC(C)(C)C)NC(=O)CCOCCOCCOCCNC(=O)OC(C)(C)C)C(=O)N=[S@@](C)(=O)Cc1cc2cc(c1)OCCCCOc1cc(F)ccc1-c1nc(ncc1F)N2]

Trifluoroacetic acid N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (1/1) (150 mg, 223 μmol) (Intermediate 44) was dissolved in DMF (20 mL). Example B3 (158 mg, 267 μmol), HATU (110 mg, 289 μmol; CAS-RN:[148893-10-1]) and DIEA (120 μl, 670 μmol; CAS-RN:[7087-68-5]) were added. The reaction was stirred at RT for 1 hour, evaporated and the residue was separated by prep. HPLC to give Intermediate 45 (199 mg, 99% purity, 79% yield) as a light-yellow foam. LC-MS (Method 3): Rₜ=5.26 min; MS (ESIpos): m/z=1132 [M+H]⁺.

Example I46: Preparation of N-(3-{2-[2-(2-aminoethoxy)ethoxy]ethoxy}propanoyl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide trifluoroacetic acid (1/1) (Intermediate 46)

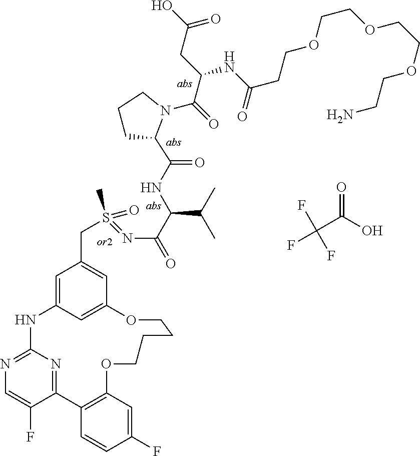 [SMILES: CC(C)[C@H](NC(=O)[C@@H]1CCCN1C(=O)[C@H](CC(=O)O)NC(=O)CCOCCOCCOCCN)C(=O)N=[S@@](C)(=O)Cc1cc2cc(c1)OCCCCOc1cc(F)ccc1-c1nc(ncc1F)N2 ; O=C(O)C(F)(F)F]

Tert-butyl (19S)-19-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (199 mg, 176 μmol) (Intermediate 45) was dissolved in DCM (20 ml), then TFA (8.0 mL) was added. The reaction was stirred for 2 hours at RT and evaporated. The residue was dissolved in ACN/water and freeze-dried to give Intermediate 46 (191 mg, 100% purity, 100% yield) as a colorless foam. LC-MS (Method 4): Rₜ=2.30 min; MS (ESIpos): m/z=975 [M+H]⁺.

Example I47: Preparation of N-(2,2-dimethyl-4,17-dioxo-3,8,11,14-tetraoxa-5-azaheptadecan-17-yl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (Intermediate 47)

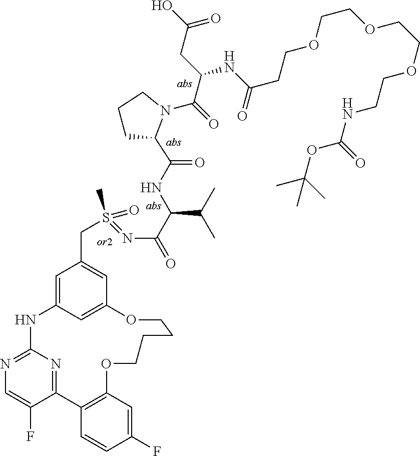 [SMILES: CC(C)[C@H](NC(=O)[C@@H]1CCCN1C(=O)[C@H](CC(=O)O)NC(=O)CCOCCOCCOCCNC(=O)OC(C)(C)C)C(=O)N=[S@@](C)(=O)Cc1cc2cc(c1)OCCCCOc1cc(F)ccc1-c1nc(ncc1F)N2]

N-(3-{2-[2-(2-aminoethoxy)ethoxy]ethoxy}propanoyl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide trifluoroacetic acid (1/1) (40.0 mg, 36.7 μmol) (Intermediate 46) was dissolved in DMF (420 μl). 1-[(tert-butoxycarbonyl)oxy]pyrrolidine-2,5-dione (9.48 mg, 44.1 μmol), DIEA (16 μl, 92 μmol; CAS-RN:[7087-68-5]) were added. The reaction was stirred for 20 h. Another 1/2 equivalent of BOC-OSu and DIEA were added and stirring was continued for 3 h at RT. The reaction was evaporated, and the residue was separated by prep. HPLC to give Intermediate 47 (36.0 mg, 100% purity, 91% yield) as a yellow foam. LC-MS (Method 3): Rₜ=4.51 min; MS (ESIpos): m/z=1075 [M+H]⁺.

Example I48: Preparation of (19S)-19-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-N,N,N,2,2-pentamethyl-4,17,21-trioxo-3,8,11,14,22-pentaoxa-5,18-diazapentacosan-25-aminium trifluoroacetate (Intermediate 48)

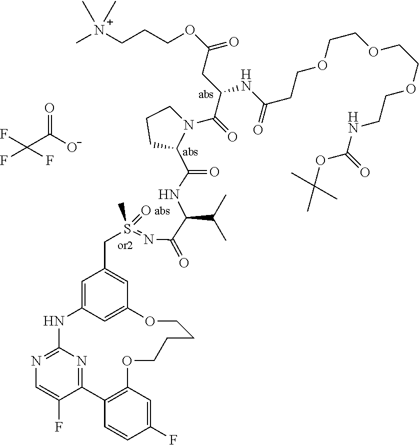 [SMILES: O=C([O-])C(F)(F)F ; CC(C)[C@H](NC(=O)[C@@H]1CCCN1C(=O)[C@H](CC(=O)OCCC[N+](C)(C)C)NC(=O)CCOCCOCCOCCNC(=O)OC(C)(C)C)C(=O)N=[S@@](C)(=O)Cc1cc2cc(c1)OCCCCOc1cc(F)ccc1-c1nc(ncc1F)N2]

N-(2,2-dimethyl-4,17-dioxo-3,8,11,14-tetraoxa-5-azaheptadecan-17-yl)-L-alpha-){[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (36.0 mg, 33.5 μmol) (Intermediate 47) was dissolved in DCM (8.3 ml). 3-Hydroxy-N,N,N-trimethylpropan-1-aminium chloride (20.6 mg, 134 μmol), NaHCO₃ (22.5 mg, 268 μmol; CAS-RN:[144-55-8]), EDCI (25.7 mg, 134 μmol; CAS-RN:[25952-53-8]) and DMAP (4.09 mg, 33.5 μmol; CAS-RN:[1 122-58-3]) were added. The reaction was stirred for 4 h. The reaction was evaporated, and the residue was separated by prep. HPLC to give Intermediate 48 (26.2 mg, 88% purity, 53% yield) as a yellow foam. LC-MS (Method 3): Rₜ=3.66 min; MS (ESIpos): m/z=1175 [M+H]⁺.

Example I49: Preparation of (14S)-1-amino-14-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-N,N,N-trimethyl-12,16-dioxo-3,6,9,17-tetraoxa-13-azaicosan-20-aminium trifluoroacetate trifluoroacetic acid (i/i/i) (Intermediate 49)

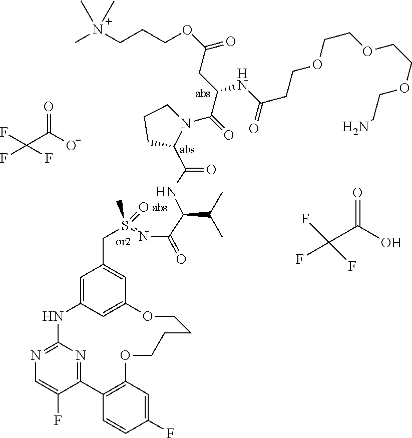 [SMILES: O=C([O-])C(F)(F)F ; CC(C)[C@H](NC(=O)[C@@H]1CCCN1C(=O)[C@H](CC(=O)OCCC[N+](C)(C)C)NC(=O)CCOCCOCCOCCN)C(=O)N=[S@@](C)(=O)Cc1cc2cc(c1)OCCCCOc1cc(F)ccc1-c1nc(ncc1F)N2 ; O=C(O)C(F)(F)F]

(19S)-19-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-N,N,N,2,2-pentamethyl-4,17,21-trioxo-3,8,11,14,22-pentaoxa-5,18-diazapentacosan-25-aminium trifluoroacetate (26.2 mg, 20.3 μmol) (Intermediate 48) was dissolved in DCM (6.0 ml), then TFA (2.0 ml) was added. The reaction was stirred for 1 hour at RT and then concentrated in vacuo. The residue was dissolved in ACN/water and freeze-dried to give Intermediate 49 (26.0 mg, 97% purity, 95% yield) as a colorless foam. LC-MS (Method 3): Rₜ=2.66 min; MS (ESIpos): m/z=1075 [M+H]⁺.

Example I50: Preparation of tert-butyl [(2S)-1-{[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]amino}-3-methyl-1-oxobutan-2-yl]carbamate (Intermediate 50)

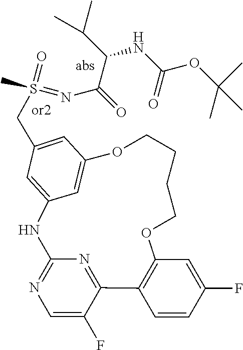 [SMILES: CC(C)[C@H](NC(=O)OC(C)(C)C)C(=O)N=[S@@](C)(=O)Cc1cc2cc(c1)OCCCCOc1cc(F)ccc1-c1nc(ncc1F)N2]

16,20-difluoro-9-[(S-methanesulfonimidoyl)methyl]-2,3,4,5-tetrahydro-12H-13,17-(azeno)-11,7-(metheno)-1,6,12,14-benzodioxadiazacyclononadecine (300 mg, 651 μmol) (Building block 14) was dissolved in DMF (40 mL). N-(tert-butoxycarbonyl)-L-valine (170 mg, 782 μmol), HATU (372 mg, 977 μmol; CAS-RN:[148893-10-1]) and DIEA (340 μl, 2.0 mmol; CAS-RN:[7087-68-5]) were added. The reaction was stirred at RT for 24 hours and then concentrated in vacuo. The residue was separated by prep. HPLC to give Intermediate 50 (351 mg, 99% purity, 81% yield) as a light-yellow foam. LC-MS (Method 1): Rₜ=2.32 min; MS (ESIpos): m/z=660 [M+H]⁺.

Example I51: Preparation of trifluoroacetic acid N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (1/1) (Intermediate 51)

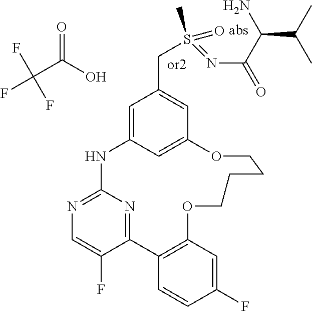 [SMILES: O=C(O)C(F)(F)F ; CC(C)[C@H](N)C(=O)N=[S@@](C)(=O)Cc1cc2cc(c1)OCCCCOc1cc(F)ccc1-c1nc(ncc1F)N2]

tert-butyl [(2S)-1-{[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]amino}-3-methyl-1-oxobutan-2-yl]carbamate (351 mg, 532 μmol) (Intermediate 50) was dissolved in DCM (30 mL), then TFA (5.0 ml) was added. The reaction was stirred for 1 hour at RT and was then concentrated in vacuo. The residue was dissolved in ACN/water and freeze-dried to give Intermediate 51 (320 mg, 100% purity, 89% yield) as a light-yellow foam. LC-MS (Method 2): Rₜ=1.30 min; MS (ESIpos): m/z=560 [M+H]⁺.

Example I52: Preparation of tert-butyl (19S)-19-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 52)

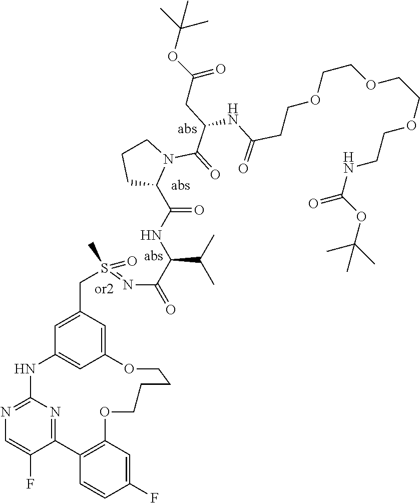 [SMILES: CC(C)[C@H](NC(=O)[C@@H]1CCCN1C(=O)[C@H](CC(=O)OC(C)(C)C)NC(=O)CCOCCOCCOCCNC(=O)OC(C)(C)C)C(=O)N=[S@@](C)(=O)Cc1cc2cc(c1)OCCCCOc1cc(F)ccc1-c1nc(ncc1F)N2]

Trifluoroacetic acid N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (1/1) (50.0 mg, 74.2 μmol) (Intermediate 51) was dissolved in DMF (8.0 ml). Example B3 (52.5 mg, 89.1 μmol), HATU (36.7 mg, 96.5 μmol; CAS-RN:[148893-10-1]) and DIEA (39 μl, 220 μmol; CAS-RN:[7087-68-5]) were added. The reaction was stirred at RT for 1 hour and was then concentrated in vacuo. The residue was separated by prep. HPLC to give Intermediate 52 (68.0 mg, 100% purity, 81% yield) as a light-yellow foam. LC-MS (Method 3): Rₜ=5.29 min; MS (ESIpos): m/z=1132 [M+H]+

Example I53: Preparation of N-(3-{2-[2-(2-aminoethoxy)ethoxy]ethoxy}propanoyl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide trifluoroacetic acid (1/1) (Intermediate 53)

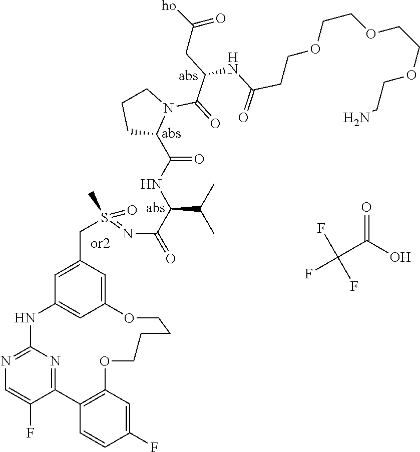

Tert-butyl (19S)-19-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (68.0 mg, 60.1 μmol) (Intermediate 52) was dissolved in DCM (6.0 ml), then TFA (3.0 ml) was added. The reaction was stirred for 2 hours at RT and was concentrated in vacuo. The residue was dissolved in ACN/water and freeze-dried to give Intermediate 53 (65.0 mg, 100% purity, 99% yield) as a colorless foam. LC-MS (Method 4): Rₜ=2.31 min; MS (ESIpos): m/z=975 [M+H]⁺.

Example I54: Preparation of N-(2,2-dimethyl-4,17-dioxo-3,8,11,14-tetraoxa-5-azaheptadecan-17-yl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (Intermediate 54)

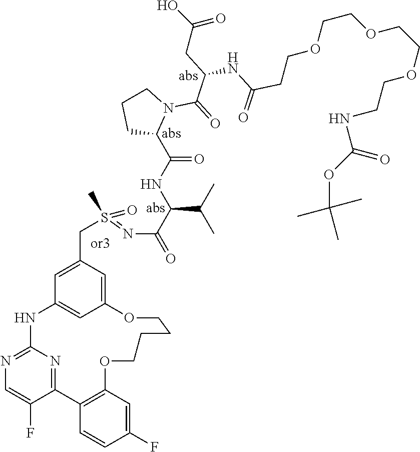 [SMILES: CC(C)[C@H](NC(=O)[C@@H]1CCCN1C(=O)[C@H](CC(=O)O)NC(=O)CCOCCOCCOCCNC(=O)OC(C)(C)C)C(=O)N=[S@@](C)(=O)Cc1cc2cc(c1)OCCCCOc1cc(F)ccc1-c1nc(ncc1F)N2]

To a solution of N-(3-{2-[2-(2-aminoethoxy)ethoxy]ethoxy}propanoyl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide trifluoroacetic acid (1/1) (45.0 mg, 41.3 μmol) (Intermediate 53) in DMF (10 ml) were added 1-[(tert-butoxycarbonyl)oxy]pyrrolidine-2,5-dione (26.7 mg, 124 μmol) and DIEA (22 μl, 120 μmol; CAS-RN:[7087-68-5]). The reaction was stirred overnight RT and concentrated in vacuo. The residue was purified via prep. HPLC to give Intermediate 54 (40.0 mg, 100% purity, 90% yield) as an amorphous residue. LC-MS (Method 3): Rₜ=4.52 min; MS (ESIpos): m/z=1075 [M+H]⁺.

Example I55: Preparation of 4-(dimethylamino)butyl (19S)-19-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 55)

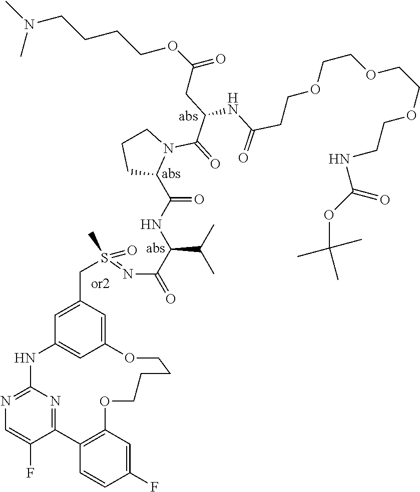 [SMILES: CC(C)[C@H](NC(=O)[C@@H]1CCCN1C(=O)[C@H](CC(=O)OCCCCN(C)C)NC(=O)CCOCCOCCOCCNC(=O)OC(C)(C)C)C(=O)N=[S@@](C)(=O)Cc1cc2cc(c1)OCCCCOc1cc(F)ccc1-c1nc(ncc1F)N2]

To a solution of N-(2,2-dimethyl-4,17-dioxo-3,8,11,14-tetraoxa-5-azaheptadecan-17-yl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (40.0 mg, 37.2 μmol) (Intermediate 54) and 4-(dimethylamino)butan-1-ol (43.6 mg, 372 μmol) in DMF (10 mL) were added EDCI (28.5 mg, 149 μmol; CAS-RN:[25952-53-8]) and DMAP (18.2 mg, 149 μmol; CAS-RN:[1122-58-3]). The solution was stirred over the weekend at RT and concentrated in vacuo. The residue was purified by prep. HPLC to give Intermediate 55 (22.5 mg, 100% purity, 51% yield) as an amorphous residue. LC-MS (Method 3): Rₜ=3.72 min; MS (ESIpos): m/z=1173 [M+H]⁺.

Example I56: Preparation of trifluoroacetic acid 4-(dimethylamino)butyl (14S)-1-amino-14-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-12-oxo-3,6,9-trioxa-13-azahexadecan-16-oate (1/1) (Intermediate 56)

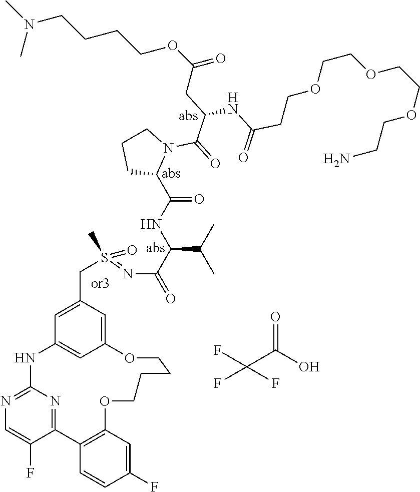 [SMILES: CC(C)[C@H](NC(=O)[C@@H]1CCCN1C(=O)[C@H](CC(=O)OCCCCN(C)C)NC(=O)CCOCCOCCOCCN)C(=O)N=[S@@](C)(=O)Cc1cc2cc(c1)OCCCCOc1cc(F)ccc1-c1nc(ncc1F)N2 ; O=C(O)C(F)(F)F]

4-(Dimethylamino)butyl (19S)-19-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (22.5 mg, 19.2 μmol) (Intermediate 55) was stirred in DCM (6.0 mL) with TFA (1.0 ml) at RT for 30 min. The reaction was concentrated in vacuo, the residue was dissolved in ACN/H₂O and lyophilized to give Intermediate 56 (23.0 mg, 96% purity, 97% yield) as an amorphous residue. LC-MS (Method 3): Rₜ=2.68 min; MS (ESIpos): m/z=1073 [M+H]⁺.

Example I57: Preparation of tert-butyl [(2S)-1-{[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]amino}-3-methyl-1-oxobutan-2-yl]carbamate (Intermediate 57)

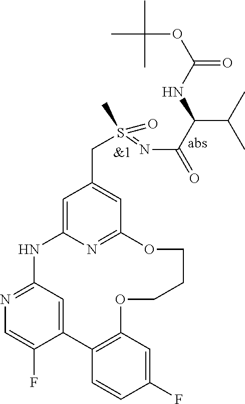 [SMILES: CC(C)[C@H](NC(=O)OC(C)(C)C)C(=O)N=[S@@](C)(=O)Cc1cc2nc(c1)OCCCOc1cc(F)ccc1-c1cc(ncc1F)N2]

15,19-Difluoro-8-[(S-methanesulfonimidoyl)methyl]-3,4-dihydro-2H,11H-10,6-(azeno)-12,16-(metheno)-1,5,11,13-benzodioxadiazacyclooctadecine (50.0 mg, 112 μmol) (B22-4) was dissolved in DMF (10 mL). N-(tert-butoxycarbonyl)-L-valine (29.2 mg, 134 μmol), HATU (63.9 mg, 168 μmol; CAS-RN:[148893-10-1]) and DIEA (59 μl, 340 μmol; CAS-RN:[7087-68-5]) were added. The reaction was stirred overnight at RT and then left to stand over the weekend. The reaction was concentrated in vacuo and purified by prep. HPLC to give Intermediate 57 (45.0 mg, 100% purity, 62% yield) as a light-yellow foam. LC-MS (Method 3): Rₜ=5.55 min; MS (ESIpos): m/z=646 [M+H]⁺.

Example I58: Preparation of trifluoroacetic acid N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (1/1) (Intermediate 58)

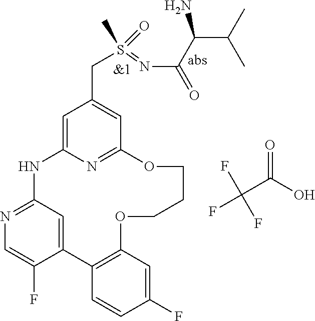

Tert-butyl [(2S)-1-{[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]amino}-3-methyl-1-oxobutan-2-yl]carbamate (40.0 mg, 61.9 μmol) (Intermediate 57) was dissolved in DCM (8.0 mL), then TFA (2.0 mL) was added. The reaction was stirred for 1 hour at RT, then concentrated in vacuo. The residue was dissolved in ACN/water and freeze-dried to give Intermediate 58 (45.1 mg, 100% purity, 110% yield) as a light-yellow foam. LC-MS (Method 3): Rₜ=3.23 min; MS (ESIpos): m/z=546 [M+H]⁺.

Example I59: Preparation of N-methyl-N-(2,2,8,11-tetramethyl-4-oxo-3-oxa-5,8,11-triazatridecan-13-yl)glycyl-L-asparaginyl-L-prolyl-N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (Intermediate 59)

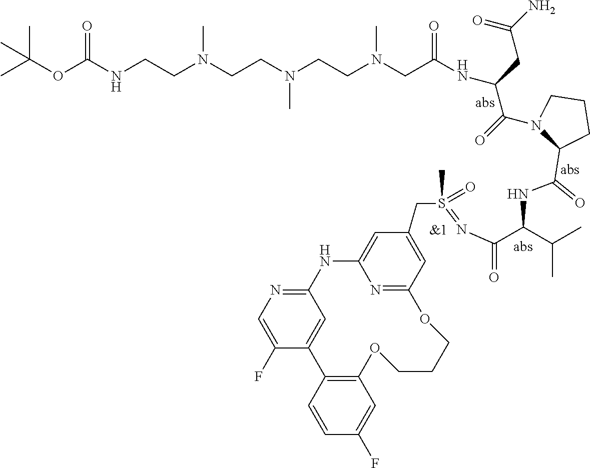 [SMILES: CC(C)[C@H](NC(=O)[C@@H]1CCCN1C(=O)[C@H](CC(N)=O)NC(=O)CN(C)CCN(C)CCN(C)CCNC(=O)OC(C)(C)C)C(=O)N=[S@@](C)(=O)Cc1cc2nc(c1)OCCCOc1cc(F)ccc1-c1cc(ncc1F)N2]

Trifluoroacetic acid N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (1/1) (30.0 mg, 45.5 μmol) (Intermediate 58) was dissolved in DMF (3.3 ml). N-methyl-N-(2,2,8,11-tetramethyl-4-oxo-3-oxa-5,8,11-triazatridecan-13-yl)glycyl-L-asparaginyl-L-proline (Building block 12) (25.4 mg, 45.5 μmol), HATU (25.9 mg, 68.2 μmol; CAS-RN:[148893-10-1]) and DIEA (16 μl, 91 μmol; CAS-RN:[7087-68-5]) were added. The reaction was stirred at RT for 3 hours and concentrated in vacuo. The residue was separated by prep. HPLC to give Intermediate 59 (22.0 mg, 100% purity, 45% yield) as a light-yellow foam. LC-MS (Method 4): Rₜ=2.32 min; MS (ESIpos): m/z=1085 [M+H]⁺.

Example I60: Preparation of trifluoroacetic acid N-{2-[{2-[(2-aminoethyl)(methyl)amino]ethyl}(methyl) amino]ethyl}-N-methylglycyl-L-asparaginyl-L-prolyl-N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (1/1) (Intermediate 60)

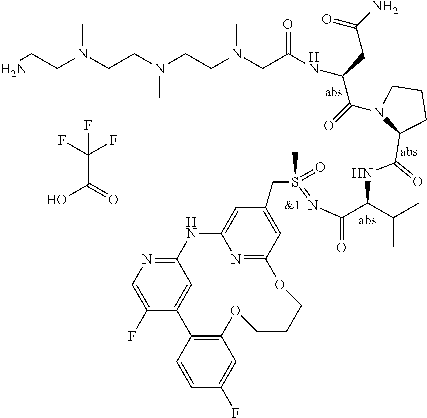 [SMILES: CC(C)[C@H](NC(=O)[C@@H]1CCCN1C(=O)[C@H](CC(N)=O)NC(=O)CN(C)CCN(C)CCN(C)CCN)C(=O)N=[S@@](C)(=O)Cc1cc2nc(c1)OCCCOc1cc(F)ccc1-c1cc(ncc1F)N2 ; O=C(O)C(F)(F)F]

N-methyl-N-(2,2,8,11-tetramethyl-4-oxo-3-oxa-5,8,11-triazatridecan-13-yl)glycyl-L-asparaginyl-L-prolyl-N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (22.0 mg, 20.3 μmol) (Intermediate 59) was dissolved in DCM (5.0 ml), then TFA (500 μl) was added. The reaction was stirred for 1 hour at RT and then concentrated in vacuo. The residue was dissolved in ACN/water and freeze-dried to give Intermediate 60 (22.0 mg, 96% purity, 95% yield) as a colorless foam. LC-MS (Method 3): Rt=2.69 min; MS (ESIpos): m/z=985 [M+H]⁺.

Example I61: Preparation of N-methyl-N-(2,2,8,11-tetramethyl-4-oxo-3-oxa-5,8,11-triazatridecan-13-yl)glycyl-L-asparaginyl-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (Intermediate 61)

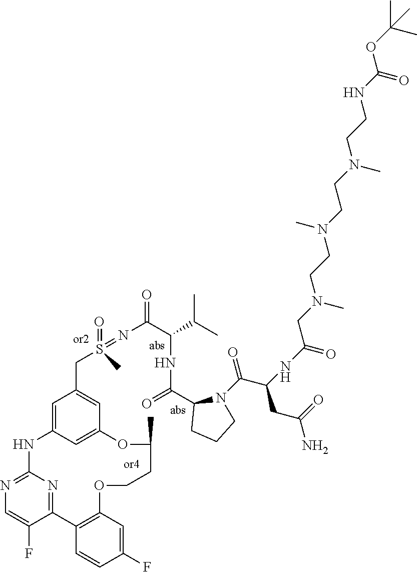 [SMILES: CC(C)[C@H](NC(=O)[C@@H]1CCCN1C(=O)[C@H](CC(N)=O)NC(=O)CN(C)CCN(C)CCN(C)CCNC(=O)OC(C)(C)C)C(=O)N=[S@@](C)(=O)Cc1cc2cc(c1)O[C@@H](C)CCOc1cc(F)ccc1-c1nc(ncc1F)N2]

To a solution of trifluoroacetic acid N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (1/1) (92.0 mg, 137 μmol) (Intermediate 29) in DMF (12 ml) were added HATU (83.1 mg, 219 μmol), N-methyl-N-(2,2,8,11-tetramethyl-4-oxo-3-oxa-5,8,11-triazatridecan-13-yl)glycyl-L-asparaginyl-L-proline (Building block 12) (83.8 mg, 150 μmol) and DIEA (71 μl, 410 μmol). Then the reaction was stirred at RT for 8 hours. The reaction was concentrated in vacuo and the residue was purified by prep. HPLC to give Intermediate 61 (93.0 mg, 100% purity, 62% yield) as a yellow amorphous residue. LC-MS (Method 1): Rₜ=1.36 min; MS (ESIpos): m/z=1099 [M+H]⁺.

Example I62: Preparation of trifluoroacetic acid N-{2-[{2-[(2-aminoethyl)(methyl)amino]ethyl}(methyl) amino]ethyl}-N-methylglycyl-L-asparaginyl-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (1/1) (Intermediate 62)

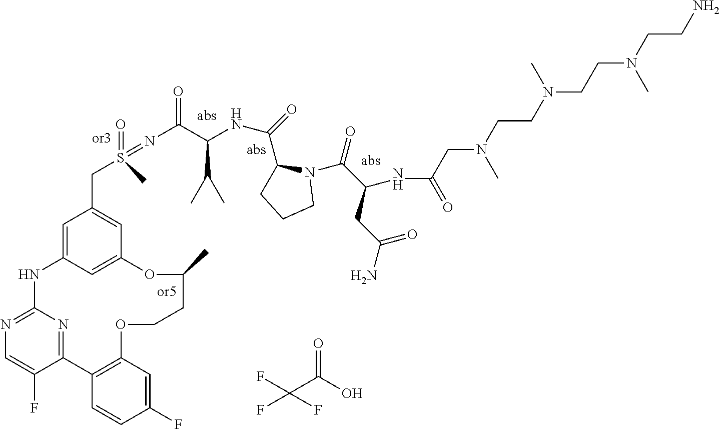 [SMILES: CC(C)[C@H](NC(=O)[C@@H]1CCCN1C(=O)[C@H](CC(N)=O)NC(=O)CN(C)CCN(C)CCN(C)CCN)C(=O)N=[S@@](C)(=O)Cc1cc2cc(c1)O[C@@H](C)CCOc1cc(F)ccc1-c1nc(ncc1F)N2 ; O=C(O)C(F)(F)F]

N-methyl-N-(2,2,8,11-tetramethyl-4-oxo-3-oxa-5,8,11-triazatridecan-13-yl)glycyl-L-asparaginyl-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (93.0 mg, 84.6 μmol) (Intermediate 61) was dissolved in DCM (10 mL) and stirred with TFA (2.0 ml) for 30 min at RT. The reaction was concentrated in vacuo, dissolved in ACN/H₂O and lyophilized to give Intermediate 62 (100 mg, 100% purity, 106% yield) as a yellow amorphous residue. LC-MS (Method 1): Rₜ=1.14 min; MS (ESIneg): m/z=997 [M−H].

Example I63: Preparation of trifluoroacetic acid (2S,22S)—N22,N24-bis(15-amino-4-oxo-7,10,13-trioxa-3-azapentadecan-1-yl)-2-[(2S)-2-{[(2S)-1-{[(S)-{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-6,9,12-trimethyl-4,16,20-trioxo-3,6,9,12,15,21-hexaazatetracosane-1,22,24-tricarboxamide (2/1) (Intermediate 63)

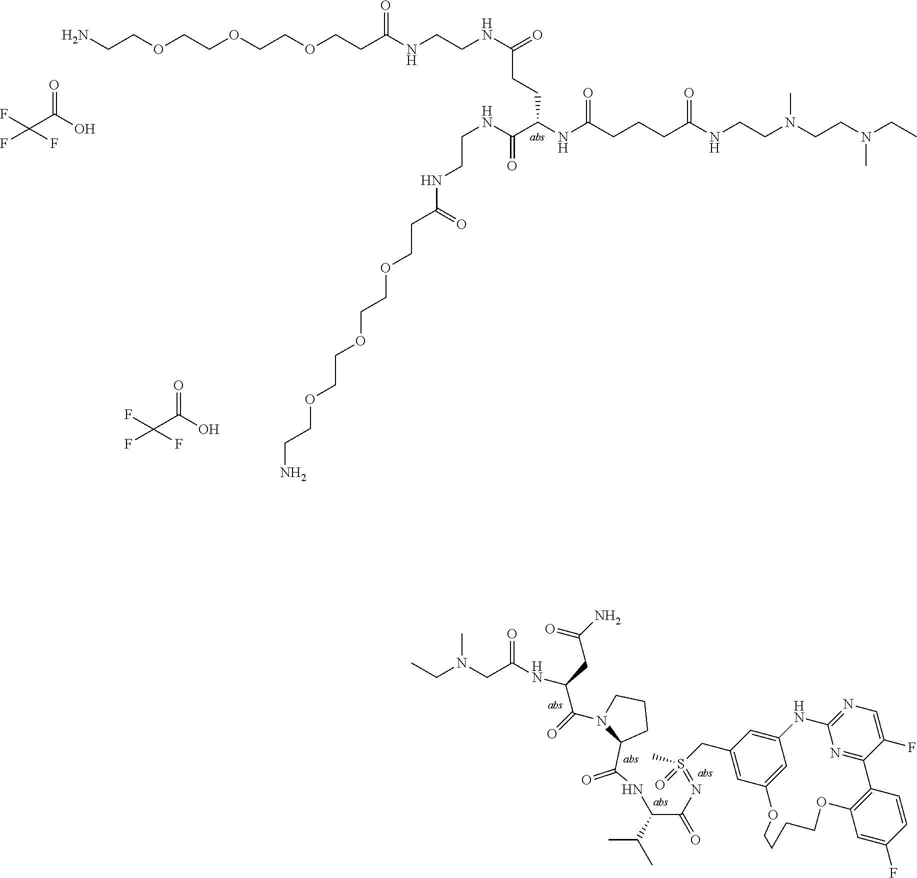

Tert-butyl [(25S)-25-({(19S)-21-amino-19-[(2S)-2-{[(2S)-1-{[(S)-{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-9,12,15-trimethyl-5,17,21-trioxo-6,9,12,15,18-pentaazahenicosanan-1-oyl}amino)-2,2-dimethyl-4,17,22,26,31-pentaoxo-3,8,11,14,34,37,40-heptaoxa-5,18,21,27,30-pentaazadotetracontan-42-yl]carbamate (9.00 mg, 98% purity, 4.56 μmol) (Intermediate 64) was dissolved in DCM (5.0 mL) and TFA (1.0 mL) was added. The reaction was stirred at RT for 30 min, concentrated in vacuo, dissolved in ACN/H₂O and lyophilized to give Intermediate 63 (7.00 mg, 95% purity, 74% yield). LC-MS (Method 3): Rₜ=2.03 min; MS (ESIpos): m/z=1731 [M+H]⁺.

Example I64: Preparation of tert-butyl [(25S)-25-({(19S)-21-amino-19-[(2S)-2-{[(2S)-1-{[(S)-{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-9,12,15-trimethyl-5,17,21-trioxo-6,9,12,15,18-pentaazahenicosanan-1-oyl}amino)-2,2-dimethyl-4,17,22,26,31-pentaoxo-3,8,11,14,34,37,40-heptaoxa-5,18,21,27,30-pentaazadotetracontan-42-yl]carbamate (Intermediate 64)

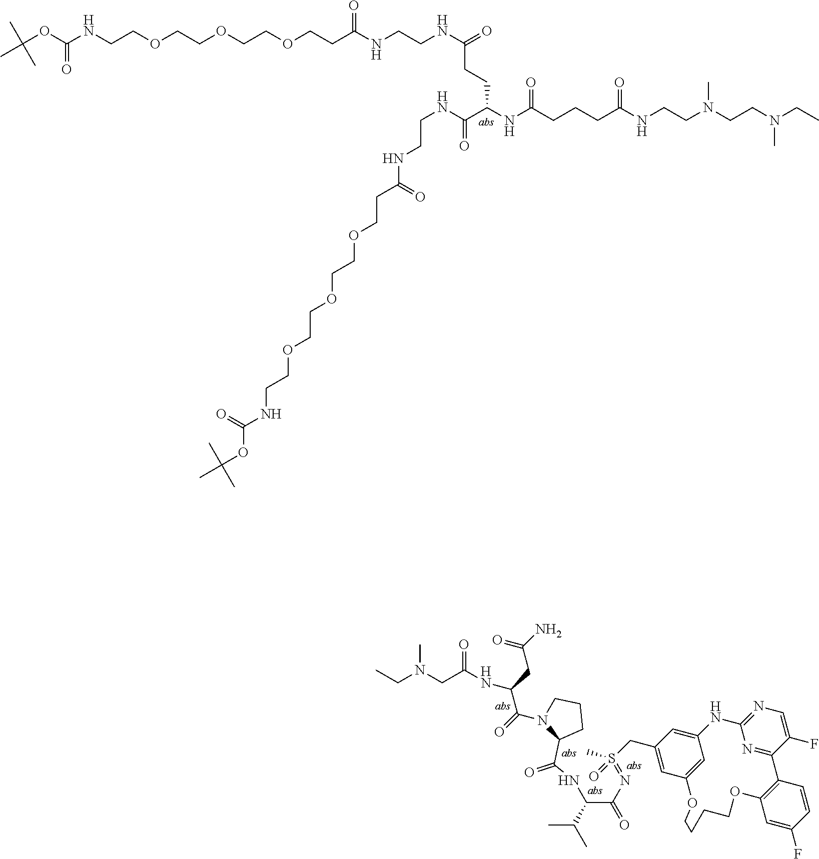

Trifluoroacetic acid (2S,22S)—N22,N24-bis(2-aminoethyl)-2-[(2S)-2-{[(2S)-1-{[(S)-{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-6,9,12-trimethyl-4,16,20-trioxo-3,6,9,12,15,21-hexaazatetracosane-1,22,24-tricarboxamide (2/1) (10.0 mg, 96% purity, 6.19 μmol) (Intermediate 65) was dissolved in DMF (5.0 mL), then educt tert-butyl {2-[2-(2-{3-[(2,5-dioxopyrrolidin-1-yl)oxy]-3-oxopropoxy}ethoxy)ethoxy]ethyl}carbamate (12.9 mg, 30.9 μmol) (Intermediate 72) and DIEA(5.4 μl, 31 μmol; CAS-RN:[7087-68-5]) were added. The reaction was stirred at RT for 1.5 h, concentrated in vacuo and the residue was purified by prep. HPLC to give Intermediate 64 (9.00 mg, 98% purity, 74% yield) as an amorphous residue. LC-MS (Method 3): Rₜ=3.33 min; MS (ESIpos): m/z=1932 [M+H]⁺.

Example 165: Preparation of trifluoroacetic acid (2S,22S)—N22,N24-bis(2-aminoethyl)-2-[(2S)-2-{[(2S)-1-{[(S)-{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-6,9,12-trimethyl-4,16,20-trioxo-3,6,9,12,15,21-hexaazatetracosane-1,22,24-tricarboxamide (2/1) (Intermediate 65)

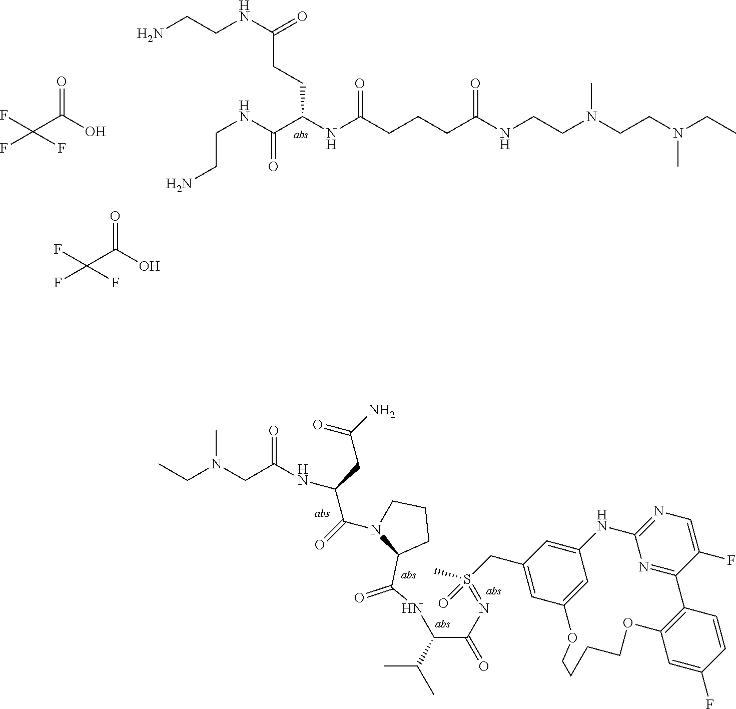

Tert-butyl {(5S,25S)-27-amino-5-[3-({2-[(tert-butoxycarbonyl)amino]ethyl}amino)-3-oxopropyl]-25-[(2S)-2-{[(2S)-1-{[(S)-{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-15,18,21-trimethyl-4,7,11,23,27-pentaoxo-3,6,12,15,18,21,24-heptaazaheptacosan-1-yl}carbamate (11.0 mg, 87% purity, 6.28 μmol) (Intermediate 66) was dissolved in DCM (5.0 mL) and TFA (1.0 mL) was added. The reaction was stirred at RT for 30 min and concentrated in vacuo. The residue was dissolved in ACN/H₂O and lyophilized to give Intermediate 65 (10.0 mg, 96% purity, 99% yield) as an amorphous residue. LC-MS (Method 3): Rₜ=2.00 min; MS (ESIpos): m/z=1325 [M+H]⁺.

Example I66: Preparation of tert-butyl {(5S,25S)-27-amino-5-[3-({2-[(tert-butoxycarbonyl)amino]ethyl}amino)-3-oxopropyl]-25-[(2S)-2-{[(2S)-1-{[(S)-{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-15,18,21-trimethyl-4,7,11,23,27-pentaoxo-3,6,12,15,18,21,24-heptaazaheptacosan-1-yl}carbamate) (Intermediate 66)

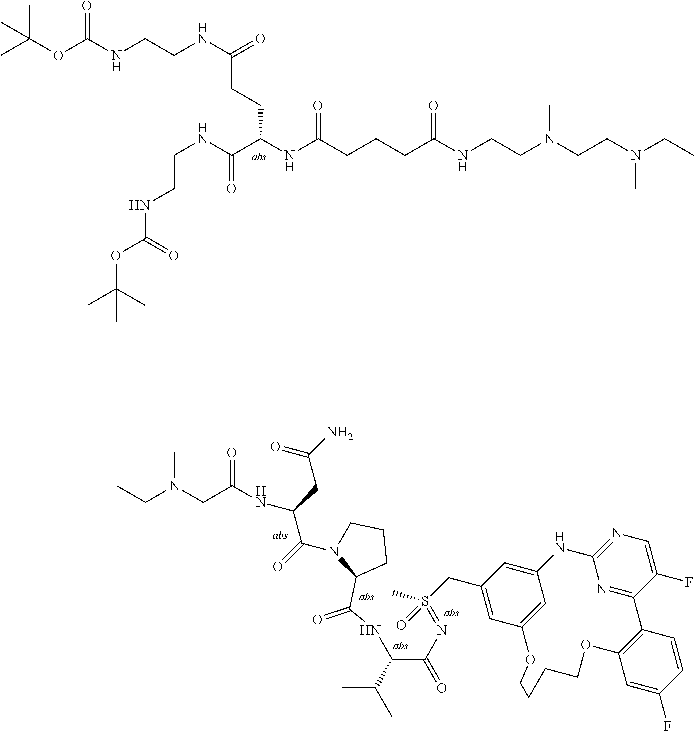

To a solution of trifluoroacetic acid N-{2-[{2-[(2-aminoethyl)(methyl)amino]ethyl}(methyl) amino]ethyl}-N-methylglycyl-L-asparaginyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (1/1) (17.0 mg, 15.3 μmol) (Intermediate 67) in DMF (6.0 mL) were added N¹,N⁵-bis{2-[(tert-butoxycarbonyl)amino]ethyl}-N²-{5-[(2,5-dioxopyrrolidin-1-yl)oxy]-5-oxopentanoyl}-L-glutamamide (17.2 mg, 74% purity, 19.9 μmol) (Intermediate 69) and DIEA (11 μl, 61 μmol; CAS-RN:[7087-68-5]). The reaction was stirred overnight at RT and concentrated in vacuo. The residue was purified by prep. HPLC and lyophilized to give Intermediate 66 (11.0 mg, 87% purity, 41% yield) as an amorphous residue. LC-MS (Method 4): Rₜ=2.43 min; MS (ESIpos): m/z=1525 [M+H]⁺.

Example I67: Preparation of trifluoroacetic acid N-{2-[{2-[(2-aminoethyl)(methyl)amino]ethyl}(methyl) amino]ethyl}-N-methylglycyl-L-asparaginyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (1/1) (Intermediate 67)

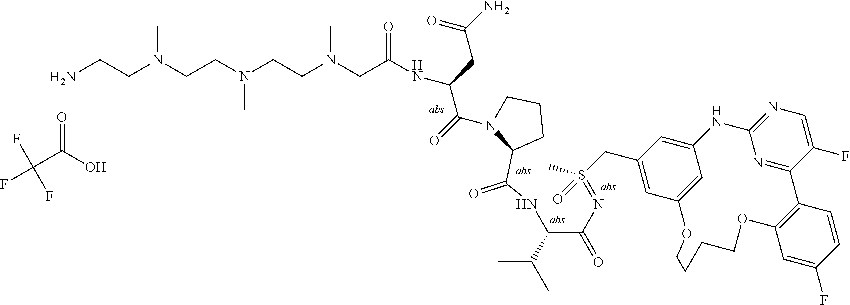

N-methyl-N-(2,2,8,11-tetramethyl-4-oxo-3-oxa-5,8,11-triazatridecan-13-yl)glycyl-L-asparaginyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (120 mg, 109 μmol) (Intermediate 68) was dissolved in DCM (8.0 mL), then TFA (2.0 mL) was added. The reaction was stirred for 1 hour at RT and concentrated in vacuo. The residue was dissolved in ACN/water and freeze-dried to give Intermediate 67 (120 mg, 100% purity, 99% yield) as a light-yellow foam. LC-MS (Method 3): Rₜ=2.59 min; MS (ESIpos): m/z=999 [M+H]⁺.

Example I68: Preparation of N-methyl-N-(2,2,8,11-tetramethyl-4-oxo-3-oxa-5,8,11-triazatridecan-13-yl)glycyl-L-asparaginyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (Intermediate 68)

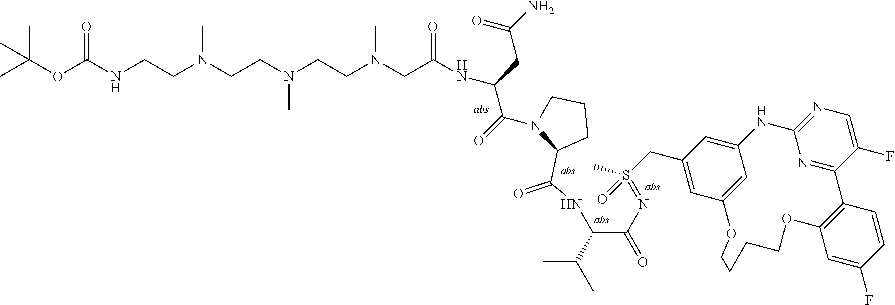 [SMILES: CC(C)[C@H](NC(=O)[C@@H]1CCCN1C(=O)[C@H](CC(N)=O)NC(=O)CN(C)CCN(C)CCN(C)CCNC(=O)OC(C)(C)C)C(=O)N=[S@@](C)(=O)Cc1cc2cc(c1)OCCCCOc1cc(F)ccc1-c1nc(ncc1F)N2]

Trifluoroacetic acid N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (1/1) (100 mg, 148 μmol) (Intermediate 10) was dissolved in DMF (11 mL). N-methyl-N-(2,2,8,11-tetramethyl-4-oxo-3-oxa-5,8,11-triazatridecan-13-yl)glycyl-L-asparaginyl-L-proline (82.8 mg, 148 μmol) (Building block 12), HATU (84.7 mg, 223 μmol; CAS-RN:[148893-10-1]) and DIEA (52 μl, 300 μmol; CAS-RN:[7087-68-5]) were added. The reaction was stirred at RT for 1 hour and then concentrated in vacuo. The residue was separated by prep. HPLC to give Intermediate 68 (120 mg, 100% purity, 74% yield) as a light-yellow foam. LC-MS (Method 4): Rₜ=2.29 min; MS (ESIpos): m/z=1099 [M+H]⁺.

Example I69: Preparation of N1,N5-bis{2-[(tert-butoxycarbonyl)amino]ethyl}-N2-{5-[(2,5-dioxopyrrolidin-1-yl)oxy]-5-oxopentanoyl}-L-glutamamide (Intermediate 69)

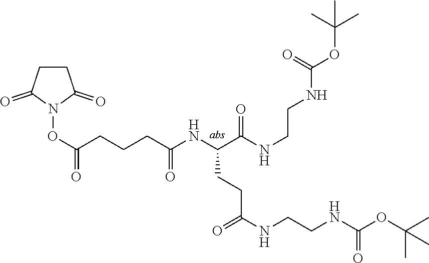 [SMILES: CC(C)(C)OC(=O)NCCNC(=O)CC[C@H](NC(=O)CCCC(=O)ON1C(=O)CCC1=O)C(=O)NCCNC(=O)OC(C)(C)C]

To a solution of N¹,N⁵-bis{2-[(tert-butoxycarbonyl)amino]ethyl}-L-glutamamide (50.0 mg, 95% purity, 110 μmol) (Intermediate 70) in DMF (10 mL) were added 1,1′-[(1,5-dioxopentane-1,5-diyl)bis(oxy)]di(pyrrolidine-2,5-dione) (72.0 mg, 221 μmol) (disuccinimidyl glutarate) and DIEA (77 μl, 440 μmol; CAS-RN:[7087-68-5]). The reaction was stirred at RT for 1 hour and was concentrated in vacuo. The residue was purified by prep. HPLC, then lyophilized to give Intermediate 69 (36.5 mg, 74% purity, 38% yield) as an amorphous residue. LC-MS (Method 1): Rₜ=1.35 min; MS (ESIpos): m/z=643 [M+H]⁺.

Example I70: Preparation of N1,N5-bis{2-[(tert-butoxycarbonyl)amino]ethyl}-L-glutamamide (Intermediate 70)

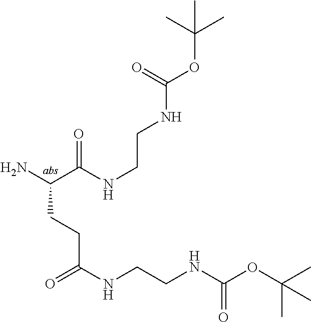 [SMILES: CC(C)(C)OC(=O)NCCNC(=O)CC[C@H](N)C(=O)NCCNC(=O)OC(C)(C)C]

Di-tert-butyl {[(2S)-2-{[(benzyloxy)carbonyl]amino}-1,5-dioxopentane-1,5-diyl]bis(azanediylethane-2,1-diyl)}biscarbamate (179 mg, 316 μmol) (Example I71) was dissolved in MeOH (40 mL) and in DCM (10 mL). Pd/C 10% was added, and the reaction was hydrogenated at RT for 1 hour. The reaction was filtered, and the mother liquor was concentrated in vacuo. The residue was dissolved in ACN/H₂O and lyophilized to give Intermediate 70 (129 mg, 95% purity, 90% yield) as a white amorphous residue. LC-MS (Method 4): Rₜ=1.37 min; MS (ESIpos): m/z=432 [M+H]⁺.

Example I71: Preparation of di-tert-butyl {[(2S)-2-{[(benzyloxy)carbonyl]amino}-1,5-dioxopentane-1,5-diyl]bis(azanediylethane-2,1-diyl)}biscarbamate (Intermediate 71)

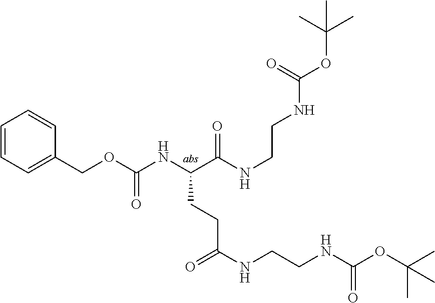 [SMILES: CC(C)(C)OC(=O)NCCNC(=O)CC[C@H](NC(=O)OCc1ccccc1)C(=O)NCCNC(=O)OC(C)(C)C]

N-[(benzyloxy)carbonyl]-L-glutamic acid (100 mg, 356 μmol) (Z-Glu-OH) was dissolved in DMF (10 mL), then tert-butyl (2-aminoethyl)carbamate (140 μl, 890 μmol), HATU (473 mg, 1.24 mmol; CAS-RN:[148893-10-1]) and DIEA (250 μl, 1.4 mmol; CAS-RN:[7087-68-5]) were added and the mixture was stirred at RT for 15 min. The reaction was concentrated in vacuo, the residue was purified by prep. HPLC to give Intermediate 71 (179 mg, 100% purity, 89% yield) as a white amorphous residue. LC-MS (Method 1): Rₜ=1.68 min; MS (ESIpos): m/z=566 [M+H]⁺.

Example I72: Preparation of tert-butyl {2-[2-(2-{3-[(2,5-dioxopyrrolidin-1-yl)oxy]-3-oxopropoxy}ethoxy)ethoxy] ethyl}carbamate (Intermediate 72)

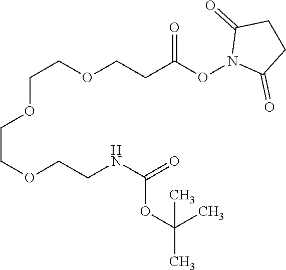 [SMILES: CC(C)(C)OC(=O)NCCOCCOCCOCCC(=O)ON1C(=O)CCC1=O]

t-Boc-N-amido-PEG3-acid: 2,2-dimethyl-4-oxo-3,8,11,14-tetraoxa-5-azaheptadecan-17-oic acid (3.00 g, 9.33 mmol) was suspended in DCM (50 ml). HO-Su: 1-hydroxypyrrolidine-2,5-dione (1.61 g, 14.0 mmol), EDCI (2.15 g, 11.2 mmol; CAS-RN:[25952-53-8]) and DMAP: 4-(dimethylamino)pyridine: (5.00 mg, 40.9 μmol; CAS-RN:[1122-58-3]) were added. The reaction was stirred for 1 hour at RT and concentrated in vacuo. The residue was purified by prep. HPLC to give Intermediate 72 (3.15 g, 90% purity, 72% yield) as a colorless oil. LC-MS (Method 1): Rₜ=1.38 min; MS (ESIpos): m/z=419 [M+H]⁺.

General Procedure for the Synthesis of PTEFb-Avb3 Integrin Conjugates with a SIL Component:

Example I73: Preparation of N-(tert-butoxycarbonyl)-L-valyl-N-[4-(hydroxymethyl)phenyl]-L-alaninamide (Intermediate 73)

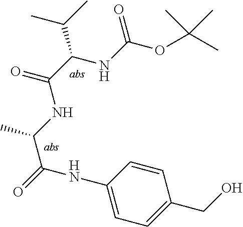 [SMILES: CC(C)[C@H](NC(=O)OC(C)(C)C)C(=O)N[C@@H](C)C(=O)Nc1ccc(CO)cc1]

To a solution of N-(tert-butoxycarbonyl)-L-valyl-L-alanine (1.00 g, 3.47 mmol) (Boc-Val-Ala-OH) in DCM (30 mL) and MeOH (15 mL) was added 4-aminobenzyl alcohol (4-aminophenyl)methanol (854 mg, 6.94 mmol) and 2-ethoxy-N-ethoxycarbonyl-1,2-dihydroquinoline (EEDQ) (1.72 g, 6.94 mmol). The reaction was stirred at RT for 24 h and then concentrated in vacuo. The residue was separated by prep. HPLC to give Intermediate 73 (1.23 g, 92% purity, 83% yield) as a colorless foam. LC-MS (Method 1): Rₜ=1.34 min; MS (ESIpos): m/z=394 [M+H]. ¹H-NMR (600 MHz, DMSO-d₆) δ[ppm]: 0.810 (2.52), 0.821 (2.66), 0.860 (3.39), 0.871 (3.31), 0.953 (0.57), 1.291 (3.92), 1.303 (3.92), 1.349 (0.61), 1.360 (1.19), 1.382 (16.00), 1.956 (0.43), 1.967 (0.42), 3.820 (0.44), 3.834 (0.61), 3.846 (0.42), 4.416 (0.60), 4.426 (6.90), 4.438 (0.62), 6.714 (0.68), 6.728 (0.65), 7.226 (2.50), 7.240 (2.88), 7.518 (3.02), 7.532 (2.70), 8.037 (0.85), 8.049 (0.84), 9.918 (1.16).

Example I74: Preparation of N-(tert-butoxycarbonyl)-L-valyl-N-[4-({[(4-nitrophenoxy)carbonyl]oxy}methyl)phenyl]-L-alaninamide (Intermediate 74)

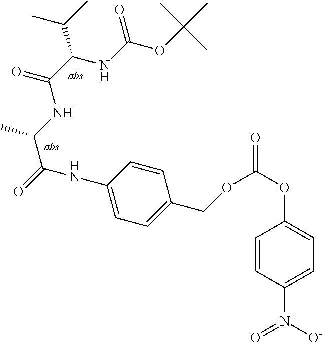 [SMILES: CC(C)[C@H](NC(=O)OC(C)(C)C)C(=O)N[C@@H](C)C(=O)Nc1ccc(COC(=O)Oc2ccc([N+](=O)[O-])cc2)cc1]

N-(tert-butoxycarbonyl)-L-valyl-N-[4-(hydroxymethyl)phenyl]-L-alaninamide (1.23 g, 3.13 mmol) (Intermediate 73) and DIEA (1.1 ml, 6.3 mmol) were dissolved in THE (200 mL), then 4-nitrophenyl carbonochloridate (945 mg, 4.69 mmol) and DMAP (10.3 mg, 83.9 μmol) were added. The reaction was stirred at RT for 1 hour, concentrated in vacuo and the residue was dissolved in ethyl acetate. The organic phase was washed with saturated NaHCO₃ solution, 5% citric acid solution and saturated sodium chloride solution. The compound was dried over MgSO₄, filtered and concentrated. The crude product was separated twice by prep. HPLC to give Intermediate 74 (380 mg, 92% purity, 20% yield) as a colorless foam. LC-MS (Method 1): Rt=2.00 min; MS (ESIpos): m/z=559 [M+H]⁺. ¹H-NMR (500 MHz, DMSO-d₆) δ[ppm]: 0.814 (2.28), 0.827 (2.43), 0.850 (0.49), 0.866 (3.00), 0.880 (2.98), 1.304 (3.66), 1.318 (3.58), 1.385 (16.00), 1.955 (0.40), 3.626 (0.73), 3.825 (0.43), 3.839 (0.59), 4.431 (0.63), 4.445 (0.44), 5.244 (5.03), 5.370 (0.50), 6.708 (0.66), 6.725 (0.63), 7.408 (2.38), 7.425 (2.67), 7.560 (3.29), 7.565 (1.01), 7.574 (1.11), 7.579 (3.55), 7.626 (2.89), 7.643 (2.32), 8.084 (0.86), 8.097 (0.79), 8.305 (3.42), 8.309 (1.01), 8.319 (1.04), 8.323 (3.25), 10.091 (1.08).

Example I75: Preparation of tert-butyl {2-[2-(2-{3-[(2,5-dioxopyrrolidin-1-yl)oxy]-3-oxopropoxy}ethoxy)ethoxy]ethyl}carbamate (Intermediate 75)

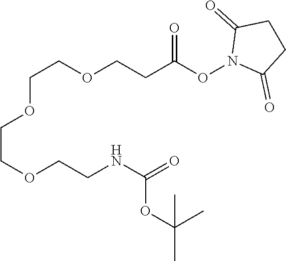 [SMILES: CC(C)(C)OC(=O)NCCOCCOCCOCCC(=O)ON1C(=O)CCC1=O]

2,2-Dimethyl-4-oxo-3,8,11,14-tetraoxa-5-azaheptadecan-17-oic acid (t-Boc-N-amido-PEG3-acid) (3.00 g, 9.33 mmol) was suspended in DCM (50 ml; CAS-RN:[75-09-2]). 1-Hydroxypyrrolidine-2,5-dione (HOSu) (1.61 g, 14.0 mmol), EDCI (2.15 g, 11.2 mmol) and DMAP (5.00 mg, 40.9 μmol; CAS-RN:[1122-58-3]) were added. The reaction was stirred for 1 hour at RT, then concentrated in vacuo. The residue was purified by prep. HPLC to give Intermediate 75 (3.15 g, 90% purity, 72% yield) as a colorless oil. LC-MS (Method 1): Rₜ=1.38 min; MS (ESIpos): m/z=419 [M+H]⁺.

Example I76: Preparation of N-(tert-butoxycarbonyl)-L-valyl-N-{4-[({[(S)-[(2-{[5-fluoro-4-(4-fluoro-2-methoxyphenyl)pyridin-2-yl]amino}pyridin-4-yl)methyl](methyl)oxo-lambda⁶-sulfanylidene]carbamoyl}oxy)methyl]phenyl}-L-alaninamide (Intermediate 76)

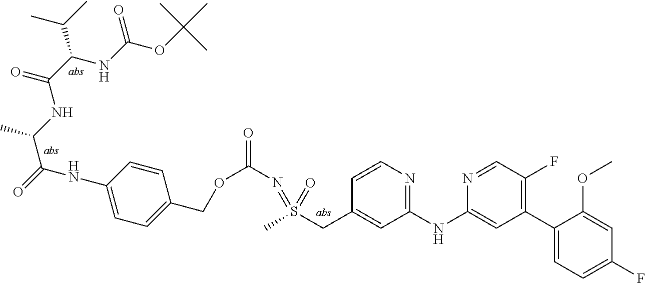 [SMILES: COc1cc(F)ccc1-c1cc(Nc2cc(C[S@](C)(=O)=NC(=O)OCc3ccc(NC(=O)[C@H](C)NC(=O)[C@@H](NC(=O)OC(C)(C)C)C(C)C)cc3)ccn2)ncc1F]

5-fluoro-4-(4-fluoro-2-methoxyphenyl)-N-{4-[(S-methanesulfonimidoyl)methyl]pyridin-2-yl}pyridin-2-amine (100 mg, 247 μmol) (Building block 5) was dissolved in DMF (20 mL). N-(tert-butoxycarbonyl)-L-valyl-N-[4-({[(4-nitrophenoxy)carbonyl]oxy}methyl)phenyl]-L-alaninamide (138 mg, 247 μmol) (Intermediate 74), 1-hydroxy-1H-benzotriazole hydrate (HOBT) (37.9 mg, 247 μmol) and DIEA (86 μl, 490 μmol) were added. The reaction was stirred at RT for 46 h. ⅓ of the amount of starting material Intermediate 74 was added again. The reaction was stirred for a further 3 h at RT and then concentrated in vacuo. The residue was separated by prep. HPLC to give Intermediate 76 (7.50 mg, 94% purity, 3% yield) as a colorless foam. LC-MS (Method 3): Rₜ=4.08 min; MS (ESIpos): m/z=824 [M+H]⁺.

Example I77: Preparation of trifluoroacetic acid L-valyl-N-{4-[({[(S)-[(2-{[5-fluoro-4-(4-fluoro-2-methoxyphenyl)pyridin-2-yl]amino}pyridin-4-yl)methyl](methyl)oxo-lambda⁶-sulfanylidene]carbamoyl}oxy)methyl]phenyl}-L-alaninamide (1/1) (Intermediate 77)

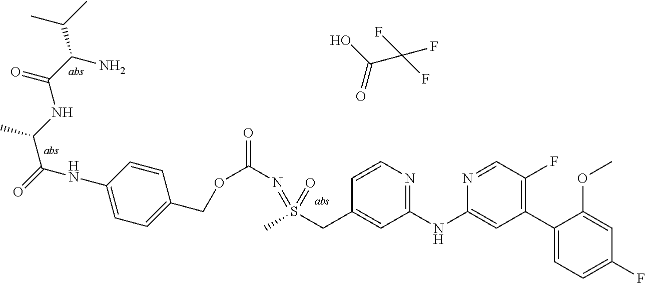 [SMILES: COc1cc(F)ccc1-c1cc(Nc2cc(C[S@](C)(=O)=NC(=O)OCc3ccc(NC(=O)[C@H](C)NC(=O)[C@@H](N)C(C)C)cc3)ccn2)ncc1F ; O=C(O)C(F)(F)F]

N-(tert-butoxycarbonyl)-L-valyl-N-{4-[({[(S)-[(2-{[5-fluoro-4-(4-fluoro-2-methoxyphenyl)pyri din-2-yl] amino}pyridin-4-yl)methyl] (methyl)oxo-lambda6-sulfanylidene]carbamoyl} oxy)methyl]phenyl}I-L-alaninamide (20.8 mg, 25.2 μmol) (Intermediate 76) was dissolved in DCM (3.1 ml), then TFA (520 μl) was added. The reaction was stirred for 1 hour at RT and then concentrated in vacuo. The residue was dissolved in ACN/water and freeze-dried to give Intermediate 77 (21.0 mg, 61% purity, 61% yield) as a yellow foam. LC-MS (Method 2): Rₜ=1.16 min; MS (ESIneg): m/z=722 [M−H]⁻

Example I78: Preparation of N-(2,2-dimethyl-4,17-dioxo-3,8,11,14-tetraoxa-5-azaheptadecan-17-yl)-L-valyl-N-{4-[({[(S)-[(2-{[5-fluoro-4-(4-fluoro-2-methoxyphenyl)pyridin-2-yl]amino}pyridin-4-yl)methyl](methyl)oxo-lambda⁶-sulfanylidene]carbamoyl}oxy)methyl]phenyl}-L-alaninamide (Intermediate 78)

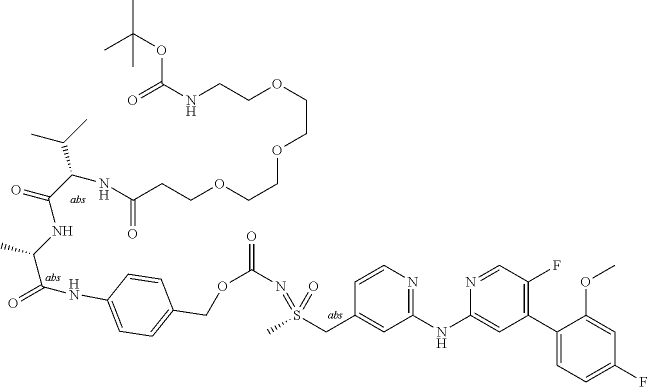 [SMILES: COc1cc(F)ccc1-c1cc(Nc2cc(C[S@](C)(=O)=NC(=O)OCc3ccc(NC(=O)[C@H](C)NC(=O)[C@@H](NC(=O)CCOCCOCCOCCNC(=O)OC(C)(C)C)C(C)C)cc3)ccn2)ncc1F]

Trifluoroacetic acid L-valyl-N-{4-[({[(S)-[(2-{[5-fluoro-4-(4-fluoro-2-methoxyphenyl)pyridin-2-yl]amino}pyridin-4-yl)methyl](methyl)oxo-lambda6-sulfanylidene]carbamoyl}oxy)methyl]phenyl}-L-alaninamide (1/1) (20.0 mg, 23.9 μmol) (Intermediate 77) was dissolved in DMF (5.0 ml). Tert-butyl {2-[2-(2-{3-[(2,5-dioxopyrrolidin-1-yl)oxy]-3-oxopropoxy}ethoxy)ethoxy]ethyl}carbamate (24.0 mg, 57.3 μmol) and DIEA (33 μl, 190 μmol) were added. The reaction was stirred at RT for 2 h and then concentrated in vacuo. The residue was separated by prep. HPLC to give Intermediate 78 (11.5 mg, 100% purity, 47% yield) as a colorless foam. LC-MS (Method 1): Rₜ=1.68 min; MS (ESIpos): m/z=1027 [M+H]⁺.

Example I79: Preparation of trifluoroacetic acid N-(3-{2-[2-(2-aminoethoxy)ethoxy]ethoxy}propanoyl)-L-valyl-N-{4-[({[(S)-[(2-{[5-fluoro-4-(4-fluoro-2-methoxyphenyl)pyridin-2-yl]amino}pyridin-4-yl)methyl](methyl)oxo-lambda⁶-sulfanylidene]carbamoyl}oxy)methyl]phenyl}-L-alaninamide (1/1) (Intermediate 79)

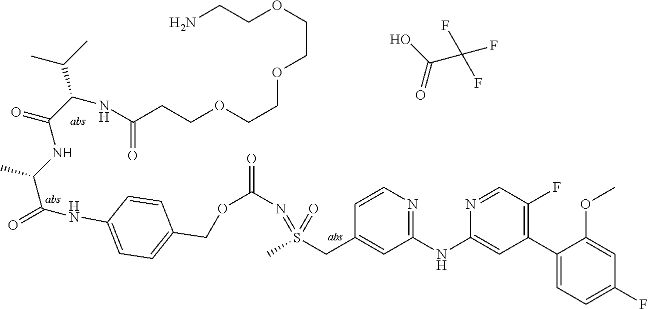 [SMILES: COc1cc(F)ccc1-c1cc(Nc2cc(C[S@](C)(=O)=NC(=O)OCc3ccc(NC(=O)[C@H](C)NC(=O)[C@@H](NC(=O)CCOCCOCCOCCN)C(C)C)cc3)ccn2)ncc1F ; O=C(O)C(F)(F)F]

N-(2,2-dimethyl-4,17-dioxo-3,8,11,14-tetraoxa-5-azaheptadecan-17-yl)-L-valyl-N-{4-[({[(S)-[(2-{[5-fluoro-4-(4-fluoro-2-methoxyphenyl)pyridin-2-yl]amino}pyridin-4-yl)methyl](methyl)oxo-lambda6-sulfanylidene]carbamoyl}oxy)methyl]phenyl}-L-alaninamide (11.5 mg, 11.2 μmol) (Intermediate 78) was dissolved in DCM (3.0 ml), then TFA (500 μl) was added. The reaction was stirred for 1 hour at RT and then concentrated in vacuo. The residue was dissolved in ACN/water and freeze-dried to give Intermediate 79 (9.20 mg, 77% purity, 61% yield) as a light-yellow foam. LC-MS (Method 4): Rₜ=1.99 min; MS (ESIpos): m/z=927 [M+H]+

General Procedure for Preparing a PTEFb-Avb3 Integrin Conjugate without a SIL Component:

Example I80: Preparation of tert-butyl [(2S)-1-{[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]amino}-3-methyl-1-oxobutan-2-yl]carbamate (Intermediate 80)

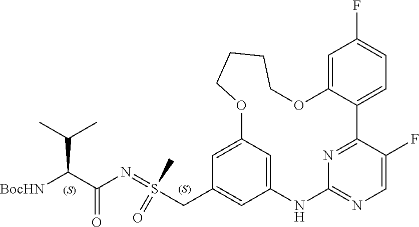

16,20-Difluoro-9-[(S-methanesulfonimidoyl)methyl]-2,3,4,5-tetrahydro-12H-13,17-(azeno)-11,7-(metheno)-1,6,12,14-benzodioxadiazacyclononadecine (300 mg, 651 μmol) (Example B14) was dissolved in DMF (40 ml). N-(tert-butoxycarbonyl)-L-valine (170 mg, 782 μmol), HATU (372 mg, 977 μmol; CAS-RN:[148893-10-1]) and DIEA (340 μl, 2.0 mmol; CAS-RN:[7087-68-5]) were added. The reaction was stirred at RT for 24 hour. The reaction was concentrated in vacuo. The residue was purified by prep. HPLC to give Intermediate 80 (351 mg, 99% purity, 81% yield) as light-yellow foam. LC-MS (Method 1): Rₜ=2.32 min; MS (ESIpos): m/z=660 [M+H]⁺.

Example I81: Preparation of trifluoroacetic acid N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (1/1) (Intermediate 81)

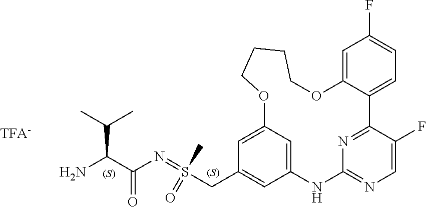

Tert-butyl [(2S)-1-{[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]amino}-3-methyl-1-oxobutan-2-yl]carbamate (351 mg, 532 μmol) (Intermediate 50) was dissolved in DCM (30 ml), then TFA (5.0 ml, 65 mmol; CAS-RN:[76-05-1]) was added. The reaction was stirred for 1 hour at RT and concentrated in vacuo. The residue was dissolved in ACN/water and freeze-dried to give Intermediate 81 (320 mg, 100% purity, 89% yield) as a light-yellow foam. LC-MS (Method 2): Rₜ=1.30 min; MS (ESIpos): m/z=560 [M+H]⁺.

Example I82: Preparation of tert-butyl (19S)-19-[(2S)-2-{[(2S)-1-{[(R*)—{[5,23-difluoro-8,13-dioxa-19,21,24-triazatetracyclo[18.3.1.1^{14,18}.0^{2,7}]pentacosa-1(24),2,4,6,14(25),15,17,20,22-nonaen-16-yl]methyl}(methyl)oxo-lambda⁶-sulfanylidene]amino}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 82)

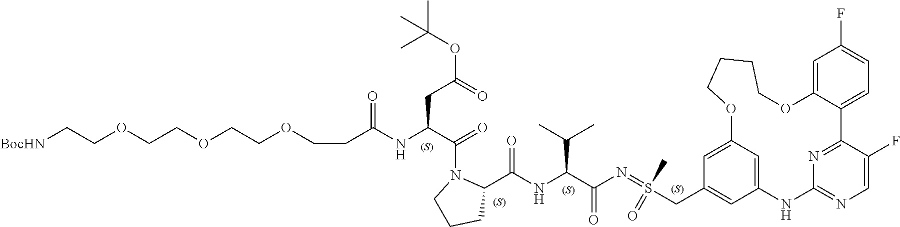

Trifluoroacetic acid N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (1/1) (50.0 mg, 74.2 μmol) (Intermediate 51) was dissolved in DMF (8.0 ml). (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (52.5 mg, 89.1 μmol) (Building block 3), HATU (36.7 mg, 96.5 μmol; CAS-RN:[148893-10-1]) and DIEA (39 μl, 220 μmol; CAS-RN:[7087-68-5]) were added. The reaction was stirred at RT for 1 hour and then concentrated in vacuo. The residue was separated by prep. HPLC to give Intermediate 82 (68.0 mg, 100% purity, 81% yield) as a light-yellow foam. LC-MS (Method 3): Rₜ=5.29 min; MS (ESIpos): m/z=1132 [M+H]⁺.

Example I83: Preparation of N-(3-{2-[2-(2-aminoethoxy)ethoxy]ethoxy}propanoyl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide trifluoroacetic acid (1/1) (Intermediate 83)

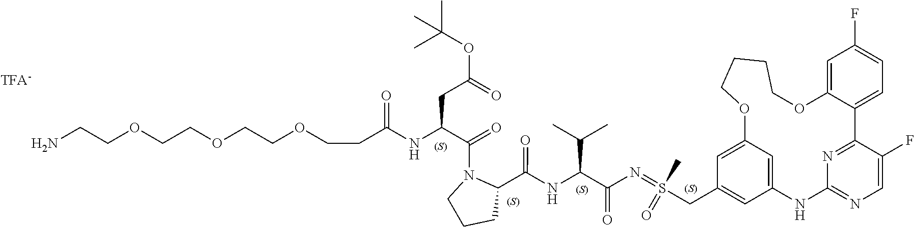

Tert-butyl (19S)-19-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (68.0 mg, 60.1 μmol) (Intermediate 52) was dissolved in DCM (6.0 ml), then TFA (3.0 ml) was added. The reaction was stirred for 2 hour at RT and concentrated in vacuo. The residue was dissolved in ACN/water and freeze-dried to give Intermediate 83 (65.0 mg, 100% purity, 99% yield) as colorless foam. LC-MS (Method 4): Rₜ=2.31 min; MS (ESIpos): m/z=975 [M+H]⁺.

Example I84: Preparation of N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (Intermediate 84)

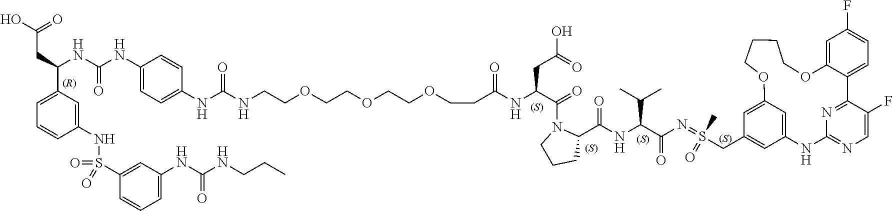

N-(3-{2-[2-(2-aminoethoxy)ethoxy]ethoxy}propanoyl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide trifluoroacetic acid (1/1) (20.0 mg, 18.4 μmol) (Intermediate 53) was dissolved in DMF (5.0 ml). (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (13.2 mg, 18.4 μmol) (Building block 1) and DIEA (64 μl, 370 μmol; CAS-RN:[7087-68-5]) were added. The reaction was stirred for 15 minutes at RT and concentrated in vacuo. The residue was separated by prep. HPLC to give Intermediate 84 (21.2 mg, 97% purity, 72% yield) as an almost colorless foam. LC-MS (Method 3): Rₜ=4.32 min; MS (ESIpos): m/z=1555 [M+H]⁺.

Compounds

Example C1. Preparation of disodium 1-[(2S)-2-(carboxylatomethyl)-17-{4-[({(1R)-2-carboxylato-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-N—[(R*)-[(3-{[4-(4-fluoro-2-methoxyphenyl)-1,3,5-triazin-2-yl]amino}phenyl)methyl](methyl) oxo-lambda⁶-sulfanylidene]-L-valinamide (Compound 1)

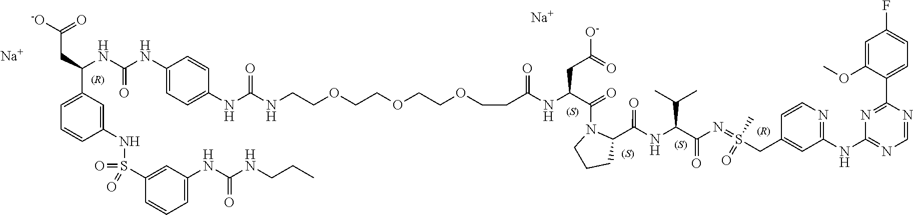

N-(3-{2-[2-(2-aminoethoxy)ethoxy]ethoxy}propanoyl)-L-alpha-aspartyl-L-prolyl-N—[(R*)-[(3-{[4-(4-fluoro-2-methoxyphenyl)-1,3,5-triazin-2-yl]amino}phenyl)methyl](methyl)oxo-lambda⁶-sulfanylidene]-L-valinamide-trifluoroacetic acid (1/1) (Intermediate 4) (33.0 mg, 93% purity, 30.2 μmol) and 1.2 eq (3R)-3-{[(4-{[(4-nitrophenoxy)carbonyl]amino}phenyl)carbamoyl] amino}-3-[3-({3-[(propylcarbamoyl)amino]benzene-1-sulfonyl}amino) phenyl] propanoic acid (Building block 1) (26.1 mg, 36.3 μmol) were dissolved in DMF (8 mL). N,N-diisopropylethylamine (110 μl) was added and the mixture was stirred at rt for 10 min. The solvent was evaporated by rotary evaporation and the residue was purified by prep. HPLC. Relevant fractions were collected, concentrated and the residue was lyophilized from ACN/H₂O (43.0 mg, 100% purity, 96%). LC-MS: Rₜ=2.80 min; MS (ESIpos): m/z=1482 [M+H]⁺. 43 mg (29.0 μmol) of the parent compound was dissolved in dioxane/H₂O 1:1 (6 mL), 120 μl of a 1.0 M sodium hydroxide solution (120 μmol) was added and the solution was lyophilized to give Compound 1 as the disodium salt (42.0 mg, 100% purity, 95% yield). LC-MS (Method 4): Rₜ=2.80 min; MS (ESIpos): m/z=1482 [M+H]⁺. ¹H NMR (600 MHz, DMSO-d₆) δ ppm 0.747-0.803 (m) 0.828 (t, J=7.43 Hz) 0.945 (d, J=6.65 Hz) 1.324-1.407 (m) 1.788-1.984 (m) 2.059-2.155 (m) 2.165-2.246 (m) 2.350-2.451 (m) 2.541 (s) 2.563-2.633 (m) 2.875-2.955 (m) 3.119-3.288 (m) 3.408-3.437 (m) 3.437-3.474 (m) 3.474-3.547 (m) 3.568 (s) 3.592-3.642 (m) 3.755-3.850 (m) 3.850-3.940 (m) 4.034-4.098 (m) 4.444-4.497 (m) 4.743-4.793 (m) 4.802 (s) 4.935-4.992 (m) 6.619-6.668 (m) 6.878-6.920 (m) 6.920-6.964 (m) 6.976-7.036 (m) 7.036-7.073 (m) 7.073-7.123 (m) 7.165 (t, J=8.27 Hz) 7.206-7.265 (m) 7.292 (t, J=8.34 Hz) 7.324-7.365 (m) 7.372-7.410 (m) 7.495 (s) 7.658-7.789 (m) 7.843-7.908 (m) 7.942-7.987 (m) 8.078 (br d, J=6.85 Hz) 8.179-8.233 (m) 8.774-8.842 (m) 8.794-8.809 (m) 9.220-9.311 (m) 9.803-9.913 (m) 10.523-10.560 (m) 11.248-11.308 (m).

Example C2. Preparation of disodium 1-[(2S)-2-(carboxylatomethyl)-17-{4-[({(1R)-2-carboxylato-1-[3-({3-[(propylcarbamoyl)amino]benzene-1-sulfonyl} amino)phenyl]ethyl}carbamoyl)amino]anilino}-4,17-dioxo-7,10,13-trioxa-3,16-diazaheptadecanan-1-oyl]-L-prolyl-N—[(S*)-[(2-{[5-fluoro-4-(4-fluoro-2-methoxyphenyl)pyridin-2-yl]amino} pyridin-4-yl)methyl](methyl)oxo-lambda⁶-sulfanylidene]-L-valinamide (Compound 2)

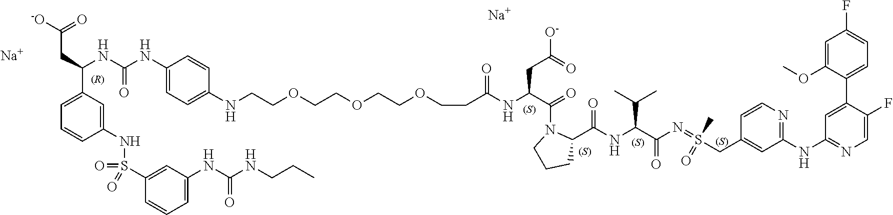

N-(3-{2-[2-(2-Aminoethoxy)ethoxy]ethoxy}propanoyl)-L-alpha-aspartyl-L-prolyl-N—[(S*)-[(2-{[5-fluoro-4-(4-fluoro-2-methoxyphenyl)pyridin-2-yl]amino}pyridin-4-yl)methyl] (methyl)oxido-lambda⁶-sulfanylidene]-L-valinamide trifluoroacetate (1:1) (Intermediate 18) (41.0 mg, 90% purity, 35.6 μmol) and 1.2 eq (3R)-3-{[(4-{[(4-nitrophenoxy)carbonyl]amino}phenyl) carbamoyl]amino}-3-[3-({3-[(propylcarbamoyl)amino]benzene-1-sulfonyl}amino) phenyl] propanoic acid (Building block 1) (29.7 mg, 95% purity, 39.2 μmol) were dissolved in DMF (6 mL). N,N-Diisopropylethylamine (124 μL) was added and the mixture was stirred overnight at rt. The solvent was evaporated by rotary evaporation and the residue was purified by prep. HPLC. Relevant fractions were collected, concentrated and the residue was lyophilized from ACN/H₂O (9.0 mg, 100% purity, 17%). LC-MS: Rₜ=2.66 min; MS (ESIpos): m/z=1499 [M+H]⁺.

9 mg (6.0 μmol) of the parent compound was dissolved in 4 ml dioxane/H₂O 1:1, 24 μl and a 1.0 M sodium hydroxide solution (24 μmol) was added, then the solution was subsequently lyophilized to give Compound 2 as the disodium salt (10.0 mg, 100% purity, 100%). LC-MS (Method 4): Rₜ=2.70 min; MS (ESIpos): m/z=1499 [M+H]⁺. ¹H NMR (600 MHz, DMSO-d₆) δ ppm 0.785 (br d, J=6.46 Hz) 0.827 (t, J=7.34 Hz) 1.323-1.398 (m) 1.810-1.984 (m) 2.070-2.150 (m) 2.168-2.270 (m) 2.333-2.460 (m) 2.518-2.565 (m) 2.565-2.656 (m) 2.879-2.968 (m) 3.140-3.292 (m) 3.224 (s) 3.364-3.476 (m) 3.476-3.540 (m) 3.564-3.641 (m) 3.728-3.862 (m) 3.794 (s) 3.949-4.071 (m) 4.405-4.479 (m) 4.754-4.809 (m) 4.852-4.943 (m) 4.943-4.991 (m) 6.600-6.686 (m) 6.882-6.926 (m) 6.926-6.970 (m) 7.008-7.071 (m) 7.071-7.130 (m) 7.174 (br d, J=8.22 Hz) 7.204-7.238 (m) 7.238-7.273 (m) 7.273-7.312 (m) 7.320-7.373 (m) 7.495 (s) 7.585 (s) 7.851-7.881 (m) 7.888 (br s) 7.906-7.956 (m) 8.075-8.144 (m) 8.182 (br s) 8.191 (s) 8.199-8.227 (m) 8.252 (s) 8.742-8.878 (m) 9.257-9.400 (m) 9.810-9.931 (m) 9.958-10.008 (m) 11.228-11.331 (m).

Example C3. Preparation of Disodium 1-[(2S)-2-(carboxylatomethyl)-17-{4-[({(1R)-2-carboxylato-1-[3-({3-[(propylcarbamoyl)amino]benzene-1-sulfonyl} amino)phenyl]ethyl}carbamoyl)amino]anilino}-4,17-dioxo-7,10,13-trioxa-3,16-diazaheptadecanan-1-oyl]-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (Compound 3)

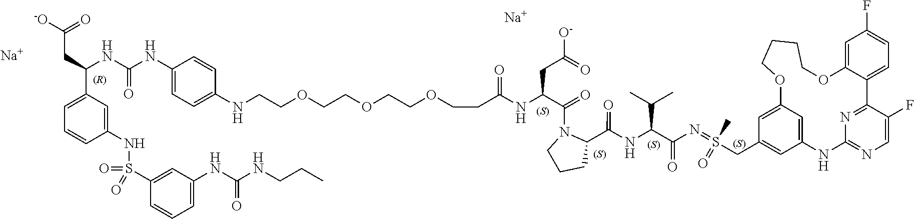

N-(3-{2-[2-(2-Aminoethoxy)ethoxy]ethoxy}propanoyl)-L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide-trifluoroacetic acid (1/1) (Intermediate 12) (12.0 mg, 11.0 μmol) and 1.2 eq (3R)-3-{[(4-{[(4-nitrophenoxy)carbonyl]amino}phenyl) carbamoyl]amino}-3-[3-({3-[(propylcarbamoyl)amino]benzene-1-sulfonyl}amino)phenyl] propanoic acid (Building block 1) (7.93 mg, 11.0 μmol) were dissolved in DMF (8 mL). N,N-Diisopropylethylamine (38 μL) was added and the mixture was stirred for 15 min at rt. The solvent was evaporated by rotary evaporation and the residue was purified by prep. HPLC. Relevant fractions were collected and evaporated to dryness. Yield: 9.5 mg (100% purity, 55% yield). LC-MS (Method 4): Rₜ=3.27 min; MS (ESIpos): m/z=1555 [M+H]⁺.

9.5 mg (6.11 μmol) of the parent compound was dissolved in 3 ml dioxane/H₂O 1:1, 12 μl of a 1.0 M sodium hydroxide solution (12 μmol) was added and the solution was subsequently lyophilized to give Compound 3 as a disodium salt. (9.0 mg, 100% purity, 92% yield). LC-MS (Method 3): Rₜ=3.27 min; MS (ESIpos): m/z=1555 [M+H]⁺. ¹H NMR (600 MHz, DMSO-d₆) δ ppm 0.801-0.873 (m) 1.359-1.434 (m) 1.791-1.846 (m) 1.863 (br s) 1.887-1.954 (m) 2.030-2.128 (m) 2.328 (td, J=6.55, 3.52 Hz) 2.372-2.446 (m) 2.541 (s) 2.564-2.654 (m) 2.685-2.751 (m) 2.937-3.027 (m) 3.177 (s) 3.194-3.243 (m) 3.407-3.445 (m) 3.445-3.475 (m) 3.475-3.509 (m) 3.518 (s) 3.548-3.602 (m) 3.602-3.661 (m) 4.054 (dd, J=8.80, 5.67 Hz) 4.084-4.190 (m) 4.271 (br s) 4.473 (dd, J=7.73, 3.62 Hz) 4.718 (s) 4.846-4.929 (m) 4.953-5.016 (m) 6.089-6.142 (m) 6.514 (s) 6.655 (s) 6.745-6.817 (m) 6.873 (t, J=8.29 Hz) 6.964 (br d, J=7.24 Hz) 7.112 (t, J=7.82 Hz) 7.150 (dd, J=11.74, 2.54 Hz) 7.192-7.243 (m) 7.284 (t, J=8.41 Hz) 7.341-7.425 (m) 7.421-7.581 (m) 7.689-7.768 (m) 7.768-7.860 (m) 7.985 (s) 8.301 (d, J=7.94 Hz) 8.342-8.418 (m) 8.578 (br s) 8.654 (d, J=1.96 Hz) 9.698 (s) 10.000-10.194 (m) 12.075-12.808 (m).

Example C4. Preparation of N-{2-[{2-[({4-[({(1R)-2-Carboxy-1-[3-({3-[(propylcarbamoyl)amino] benzene-1-sulfonyl}amino) phenyl]ethyl} carbamoyl)amino] phenyl}carbamoyl)(methyl)amino] ethyl} (methyl)amino]ethyl}-N-methylglycyl-L-asparaginyl-L-prolyl-N—[(S)-[(2-{[5-fluoro-4-(4-fluoro-2-methoxyphenyl) pyridine-2-yl]amino} pyridin-4-yl)methyl](methyl)oxo-lambda⁶-sulfanylidene]-L-valinamide (Compound 4)

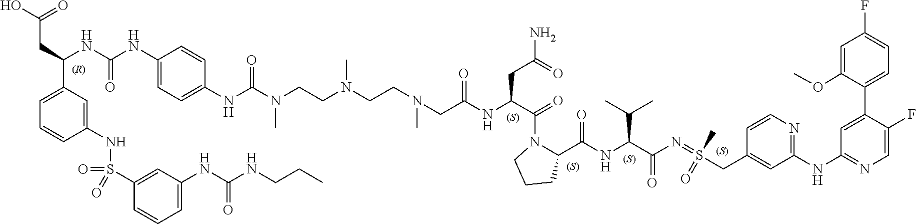

Trifluoroacetic acid N-methyl-N-(2-{methyl[2-(methylamino)ethyl]amino} ethyl)glycyl-L-asparaginyl-L-prolyl-N—[(S)-[(2-{[5-fluoro-4-(4-fluoro-2-methoxyphenyl)pyridin-2-yl]amino} pyridin-4-yl)methyl](methyl)oxo-lambda⁶-sulfanylidene]-L-valinamide (1/1) (Intermediate 15) (10 mg, 9.9 μmol) and 1.1 eq (3R)-3-{[(4-{[(4-nitrophenoxy)carbonyl]amino} phenyl) carbamoyl]amino}-3-[3-({3-[(propylcarbamoyl)amino]benzene-1-sulfonyl}amino)phenyl] propanoic acid (Building block 1) (7.81 mg, 10.8 μmol) were dissolved in DMF (4 mL). N,N-Diisopropylethylamine (34 μL) was added and the mixture was stirred for 30 min at rt. The solvent was evaporated by rotary evaporation and the residue was purified by prep. HPLC. Relevant fractions were collected and evaporated to dryness to give Compound 4 (4.4 mg, 100% purity, 30% yield). LC-MS (Method 4): Rₜ=2.39 min; MS (ESIpos): m/z=740 [M+2H]²⁺. ¹H NMR (600 MHz, DMSO-d₆) δ ppm 0.791 (d, J=6.65 Hz) 0.819 (br d, J=6.85 Hz) 0.856 (t, J=7.43 Hz) 1.218-1.255 (m) 1.398-1.459 (m) 1.830-1.916 (m) 1.942-2.003 (m) 2.071-2.132 (m) 2.382-2.452 (m) 2.522 (br d, J=1.37 Hz) 2.541 (s) 2.560-2.625 (m) 2.625-2.654 (m) 2.845 (s) 2.995 (s) 3.010-3.058 (m) 3.214-3.414 (m) 3.598-3.771 (m) 3.780-3.810 (m) 3.800 (s) 4.095 (br dd, J=9.00, 5.28 Hz) 4.463-4.494 (m) 4.862-4.901 (m) 4.901-4.950 (m) 4.975-5.021 (m) 6.263-6.288 (m) 6.707 (br d, J=8.41 Hz) 6.878-6.949 (m) 6.949-6.998 (m) 7.109 (dd, J=11.64, 2.45 Hz) 7.131-7.172 (m) 7.215-7.261 (m) 7.261-7.282 (m) 7.282-7.322 (m) 7.351 (t, J=7.60 Hz) 7.392-7.446 (m) 7.566 (s) 7.776-7.812 (m) 8.060 (s) 8.201-8.225 (m) 8.247 (s) 8.441 (s) 8.819 (s) 9.858-10.120 (m) 10.264 (s) 12.115-12.601 (m).

Example C5. Preparation of N-{2-[{2-[({4-[({(1R)-2-Carboxy-1-[3-({3-[(propylcarbamoyl)amino]benzene-1-sulfonyl}amino) phenyl]ethyl} carbamoyl)amino]phenyl}carbamoyl)(methyl)amino]ethyl} (methyl)amino]ethyl}-N-methylglycyl-L-asparaginyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (Compound 5)

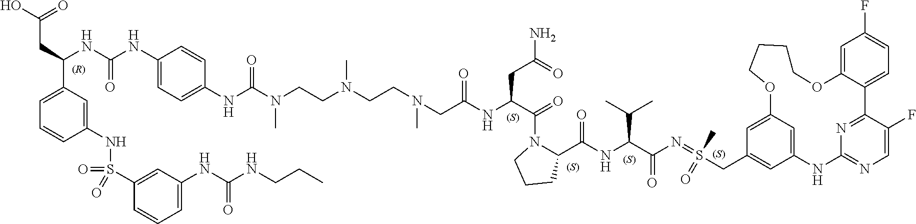

Trifluoroacetic acid-N-methyl-N-(2-{methyl[2-(methylamino)ethyl]amino}ethyl) glycyl-L-asparaginyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (1/1) (Intermediate 14) (10.0 mg, 9.34 μmol) and 1.1 eq (3R)-3-{[(4-{[(4-nitrophenoxy)carbonyl]amino}phenyl) carbamoyl]amino}-3-[3-({3-[(propylcarbamoyl)amino]benzene-1-sulfonyl}amino)phenyl] propanoic acid (Building block 1) (7.4 mg, 10.3 μmol) were dissolved in DMF (4 mL). N,N-Diisopropylethylamine (33 μL) was added and the mixture was stirred for 30 min at rt. The solvent was evaporated by rotary evaporation and the residue was purified by prep. HPLC. Relevant fractions were collected and evaporated to dryness to give Compound 5 (14 mg, 97% purity, 94%). LC-MS (Method 4): Rₜ=2.77 min; MS (ESIpos): m/z=1536 [M+H]⁺.

Example C6: Preparation of N-{2-[{2-[({4-[({(1R)-2-Carboxy-1-[3-({3-[(propylcarbamoyl)amino]benzene-1-sulfonyl}amino) phenyl]ethyl}carbamoyl)amino]phenyl}carbamoyl)(methyl)amino]ethyl}(methyl)amino]ethyl}-N-methylglycyl-L-asparaginyl-L-prolyl-N—[(R or S*)—{[(4R or S*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda⁶-sulfanylidene]-L-valinamide (*single diastereomer) (Compound 6)

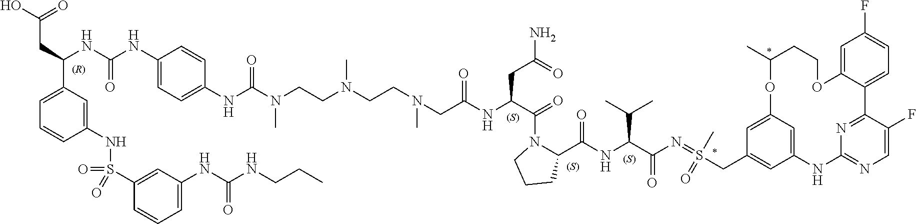

Trifluoroacetic acid-N-methyl-N-(2-{methyl[2-(methylamino)ethyl]amino}ethyl) glycyl-L-asparaginyl-L-prolyl-N—[(R or S*)—{[(4R or S*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda⁶-sulfanylidene]-L-valinamide (1/1) (* single diastereomer) (Intermediate 31) (10.0 mg, 9.3 μmol) and 1.2 eq (3R)-3-{[(4-{[(4-nitrophenoxy)carbonyl] amino}phenyl) carbamoyl]amino}-3-[3-({3-[(propylcarbamoyl)amino]benzene-1-sulfonyl} amino)phenyl]propanoic acid (Building block 1) (13.3 mg, 18.5 μmol) were dissolved in DMF (5 mL). N,N-Diisopropylethylamine (8 μL) was added and the mixture was stirred for 15 min at rt. The solvent was evaporated by rotary evaporation and the residue was purified by prep. HPLC. Relevant fractions were collected and evaporated to dryness. The residue was dissolved in ACN/H₂O and lyophilized to give Compound 6 (5.0 mg, 100% purity, 35%). LC-MS (Method 4): Rₜ=2.89 min; MS (ESIpos): m/z=1536 [M+H]⁺. ¹H NMR (600 MHz, DMSO-d₆) δ ppm 0.815-0.838 (m, 3H) 0.846 (br d, J=2.93 Hz, 3H) 0.852-0.883 (m, 3H) 1.398-1.460 (m, 5H) 1.681-1.755 (m, 1H) 1.805-1.918 (m, 3H) 1.928-2.010 (m, 1H) 2.103 (br dd, J=13.20, 6.94 Hz, 1H) 2.329 (br t, J=12.23 Hz, 1H) 2.367-2.479 (m, 3H) 2.552-2.623 (m, 2H) 2.639 (br d, J=7.04 Hz, 2H) 2.804-2.865 (m, 3H) 2.993 (s, 4H) 3.033 (q, J=6.52 Hz, 3H) 3.193 (s, 4H) 3.210-3.312 (m, 5H) 4.102 (br dd, J=8.61, 5.87 Hz, 2H) 4.365 (br dd, J=10.86, 5.97 Hz, 1H) 4.444-4.507 (m, 2H) 4.682-4.741 (m, 2H) 4.892-4.939 (m, 1H) 4.999 (q, J=7.24 Hz, 1H) 6.256 (br t, J=5.58 Hz, 1H) 6.485 (s, 1H) 6.697 (br d, J=7.82 Hz, 1H) 6.735 (s, 1H) 6.891 (br d, J=8.02 Hz, 1H) 6.927 (td, J=8.22, 2.15 Hz, 1H) 6.965-7.008 (m, 2H) 7.138-7.157 (m, 1H) 7.162 (d, J=2.15 Hz, 1H) 7.244 (br d, J=8.80 Hz, 2H) 7.243-7.261 (m, 1H) 7.258-7.291 (m, 1H) 7.291-7.323 (m, 2H) 7.345-7.383 (m, 1H) 7.384-7.421 (m, 1H) 7.421-7.447 (m, 1H) 7.627 (td, J=7.73, 4.70 Hz, 1H) 7.785 (br d, J=9.00 Hz, 1H) 8.060 (s, 1H) 8.245 (s, 1H) 8.431 (s, 1H) 8.679 (d, J=2.74 Hz, 1H) 8.706 (s, 1H) 8.797 (s, 1H) 9.744 (s, 1H) 10.263 (s, 1H) 12.107-12.475 (m, 1H).

Example C7: Preparation of sodium N-{14-[4-({[(1R)-2-carboxylato-1-{3-[({3-[(propylcarbamoyl)amino]phenyl}sulfonyl) amino]phenyl}ethyl]carbamoyl}amino)anilino]-14-oxo-4,7,10-trioxa-13-azatetradecan-1-oyl}-L-valyl-N⁵-carbamoyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxido-lambda⁶-sulfanylidene]-L-ornithinamide (Compound 7)

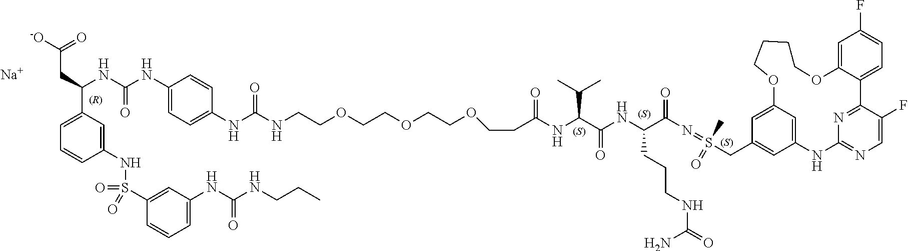

Trifluoroacetic acid N-(3-{2-[2-(2-aminoethoxy)ethoxy]ethoxy}propanoyl)-L-valyl-N⁵-carbamoyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-ornithinamide (1/1) (Intermediate 21) (10.0 mg, 9.67 μmol) and 1.8 eq (3R)-3-{[(4-{[(4-nitrophenoxy)carbonyl]amino}phenyl)carbamoyl]amino}-3-[3-({3-[(propylcarbamoyl) amino]benzene-1-sulfonyl}amino)phenyl] propanoic acid (Building block 1) (12.5 mg, 17.4 μmol) were dissolved in DMF (6 mL). N,N-Diisopropylethylamine (8 μL) was added and the mixture was stirred for 30 min at rt. The solvent was evaporated by rotary evaporation and the residue was purified by prep. HPLC. Relevant fractions were collected and evaporated to dryness. Yield: 8 mg (100% purity, 55% yield). LC-MS: Rₜ=3.19 min; MS (ESIpos): m/z=1500 [M+H]⁺.

8 mg (5.3 μmol) of the parent compound was dissolved in 4 ml dioxane/H₂O 1:1, 5.3 μl of a 1.0 M sodium hydroxide solution (5.3 μmol) was added and the solution was lyophilized to give Compound 7 as the sodium salt (6.0 mg, 100% purity, 74% yield). LC-MS (Method 4): Rₜ=3.18 min; MS (ESIpos): m/z=1500 [M+H]⁺. ¹H NMR (600 MHz, DMSO-d₆) δ ppm 0.808 (d, J=6.65 Hz) 0.844 (d, J=2.15 Hz) 0.851-0.882 (m) 1.218-1.255 (m) 1.345-1.460 (m) 1.498-1.575 (m) 1.652-1.727 (m) 1.842 (br d, J=1.17 Hz) 1.826-1.902 (m) 1.938-2.006 (m) 2.351-2.481 (m) 2.541 (s) 2.588-2.672 (m) 2.635 (br d, J=6.65 Hz) 2.860-2.945 (m) 3.031 (q, J=6.72 Hz) 3.125 (s) 3.177-3.298 (m) 3.355-3.527 (m) 3.566-3.639 (m) 4.083-4.170 (m) 4.242-4.319 (m) 4.723 (br d, J=5.67 Hz) 4.977-5.021 (m) 5.366 (br s) 5.873-5.916 (m) 6.044-6.084 (m) 6.191-6.232 (m) 6.507 (s) 6.624 (br d, J=8.02 Hz) 6.683 (s) 6.873 (t, J=8.20 Hz) 6.906 (br d, J=7.04 Hz) 6.984 (d, J=7.78 Hz) 7.129-7.172 (m) 7.188 (br s) 7.203 (s) 7.208-7.223 (m) 7.231-7.301 (m) 7.368-7.430 (m) 7.758 (d, J=9.00 Hz) 8.003 (s) 8.016-8.070 (m) 8.328 (s) 8.348 (s) 8.657 (d, J=1.96 Hz) 8.758 (s) 9.838 (s) 10.266 (s) 12.237-12.319 (m).

Example C8: Preparation of sodium N-{14-[4-({[(1R)-2-carboxylato-1-{3-[({3-[(propylcarbamoyl)amino]phenyl}sulfonyl) amino]phenyl}ethyl]carbamoyl} amino)anilino]-14-oxo-4,7,10-trioxa-13-azatetradecan-1-oyl}-L-alanyl-N-methyl-L-alanyl-N′—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxido-lambda⁶-sulfanylidene]-L-aspartamide (Compound 8)

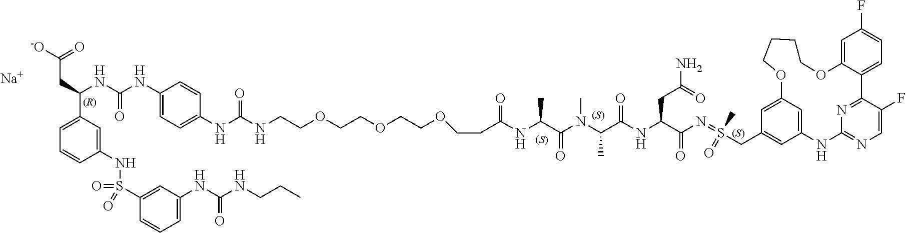

Trifluoroacetic acid N-(3-{2-[2-(2-aminoethoxy)ethoxy]ethoxy}propanoyl)-L-alanyl-N-methyl-L-alanyl-N′—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-aspartamide (1/1) (Intermediate 27) (10.0 mg, 9.54 μmol)) and 1.2 eq (3R)-3-{[(4-{[(4-nitrophenoxy)carbonyl]amino}phenyl) carbamoyl]amino}-3-[3-({3-[(propylcarbamoyl)amino] benzene-1-sulfonyl}amino)phenyl] propanoic acid (Building block 1) (7.55 mg, 10.5 μmol) were dissolved in DMF (4 mL). N,N-Diisopropylethylamine (33 μL) was added and the mixture was stirred for 30 min at rt. The solvent was evaporated by rotary evaporation and the residue was purified by prep. HPLC. Relevant fractions were collected and evaporated to dryness. Yield: 11.7 mg (100% purity, 81% yield). LC-MS: Rₜ=3.06 min; MS (ESIpos): m/z=1514 [M+H]⁺.

11.7 mg (7.7 μmol) of the parent compound was dissolved in 5 mL dioxane/H₂O 1:1, 7.7 μL of a 1.0 M sodium hydroxide solution (7.7 μmol) was added and the solution was lyophilized to give Compound 8 as the sodium salt (11.0 mg, 100% purity, 93% yield). LC-MS (Method 4): Rₜ=3.07 min; MS (ESIpos): m/z=1514 [M+H]⁺. ¹H NMR (600 MHz, DMSO-d₆) δ ppm 0.848 (t, J=7.43 Hz) 1.134-1.227 (m) 1.353-1.464 (m) 1.803-1.912 (m) 2.316-2.415 (m) 2.519-2.563 (m) 2.541 (s) 2.563-2.654 (m) 2.834 (s) 2.958-3.050 (m) 3.099-3.135 (m) 3.139 (s) 3.190-3.241 (m) 3.404-3.443 (m) 3.443-3.480 (m) 3.480-3.505 (m) 3.515 (s) 3.547-3.612 (m) 4.088-4.179 (m) 4.219-4.320 (m) 4.381-4.526 (m) 4.648-4.766 (m) 4.953-5.010 (m) 5.010-5.065 (m) 6.050-6.103 (m) 6.510 (br s) 6.671 (s) 6.795-6.868 (m) 6.868-6.926 (m) 6.974 (br d, J=7.43 Hz) 7.109-7.179 (m) 7.194 (br s) 7.192-7.231 (m) 7.216-7.259 (m) 7.270 (s) 7.275-7.304 (m) 7.337-7.448 (m) 7.510-7.675 (m) 7.742 (br d, J=8.02 Hz) 7.863-7.971 (m) 7.977-8.009 (m) 8.028 (br s) 8.156 (br d, J=8.02 Hz) 8.336 (s) 8.435-8.555 (m) 8.635-8.679 (m) 9.744 (s) 10.095-10.242 (m) 12.162-12.513 (m).

Example C9: Preparation of N-{2-[{2-[({4-[({(1S)-2-Carboxy-1-[3-({3-[(propylcarbamoyl)amino]benzene-1-sulfonyl}amino) phenyl]ethyl}carbamoyl)amino]phenyl}carbamoyl)(methyl)amino] ethyl}(methyl)amino]ethyl}-N-methylglycyl-L-asparaginyl-L-prolyl-N—[(S)-[(2-{[5-fluoro-4-(4-fluoro-2-methoxyphenyl) pyridine-2-yl]amino}pyridin-4-yl)methyl](methyl)oxo-lambda⁶-sulfanylidene]-L-valinamide (Compound 9)

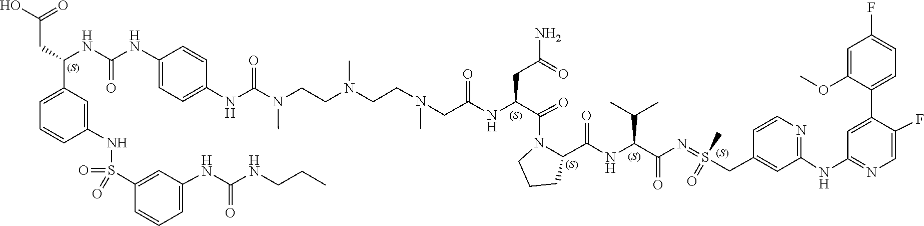

Compound 9 is the epimer of Compound 4 and was synthesized in analogy to Example C4 employing the enantiomeric Building block 2 instead of Building block 1. LC-MS (Method 4): Rₜ=2.37 min; MS (ESIpos): m/z=740 [M+2H]²⁺.

Example C10: Preparation of N-{2-[{2-[({4-[({(1S)-2-Carboxy-1-[3-({3-[(propylcarbamoyl)amino]benzene-1-sulfonyl}amino) phenyl]ethyl}carbamoyl)amino]phenyl}carbamoyl)(methyl)amino]ethyl}(methyl)amino]ethyl}-N-methylglycyl-L-asparaginyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide

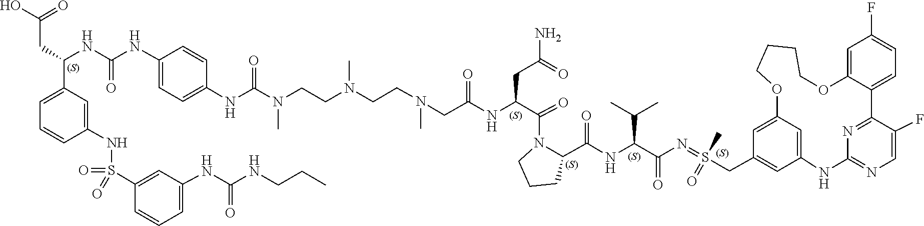

Compound 10 is the epimer of Compound 5 and was synthesized in analogy to Compound 5 using the enantiomeric Building block 2 instead of Building block 1. LC-MS (Method 4): Rₜ=2.84 min; MS (ESIpos): m/z=768 [M+2H]²⁺.

Example C11: Preparation of N-{2-[{2-[({4-[({(1S)-2-Carboxy-1-[3-({3-[(propylcarbamoyl)amino]benzene-1-sulfonyl}amino) phenyl]ethyl}carbamoyl)amino]phenyl}carbamoyl)(methyl)amino]ethyl}(methyl)amino]ethyl}-N-methylglycyl-L-asparaginyl-L-prolyl-N—[(R or S*)—{[(4R or S*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda⁶-sulfanylidene]-L-valinamide (*single diastereomer) (Compound 11)

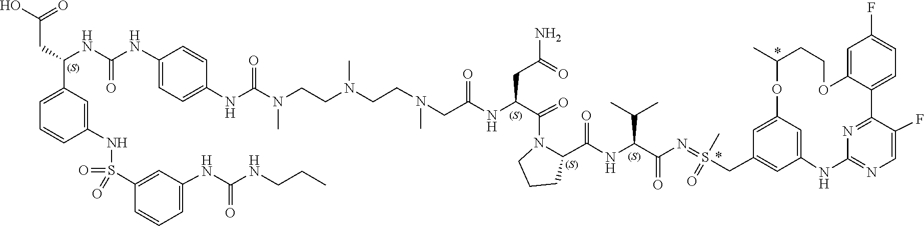

Compound 11 is the epimer of Compound 6 and was synthesized in analogy to Compound 6 using the enantiomeric Building block 2 instead of Building block 1. Yield: 5.0 mg, 100% purity, 35%. LC-MS (Method 4): Rₜ=2.89 min; MS (ESIpos): m/z=1536 [M+H]⁺.

Example C12: Preparation of Sodium N-{14-[4-({[(1S)-2-carboxylato-1-{3-[({3-[(propylcarbamoyl)amino]phenyl}sulfonyl) amino]phenyl}ethyl]carbamoyl}amino)anilino]-14-oxo-4,7,10-trioxa-13-azatetradecan-1-oyl}-L-valyl-N⁵-carbamoyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxido-lambda⁶-sulfanylidene]-L-ornithinamide (Compound 12)

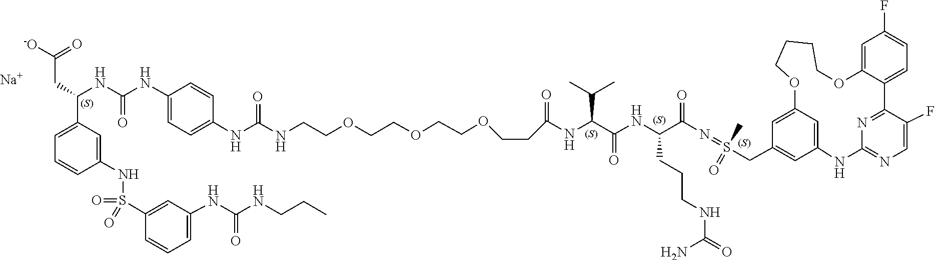

Compound 12 is the epimer of Compound 7 and was synthesized in analogy to Compound 7 employing the enantiomeric Building block 2 instead of Building block 1. Yield: 4 mg (100% purity, 27% yield). LC-MS (Method 4): Rₜ=3.19 min; MS (ESIpos): m/z=1500 [M+H]⁺.

Example C13: Preparation of Sodium N-{14-[4-({[(1S)-2-carboxylato-1-{3-[({3-[(propylcarbamoyl)amino]phenyl}sulfonyl) amino]phenyl}ethyl]carbamoyl}amino)anilino]-14-oxo-4,7,10-trioxa-13-azatetradecan-1-oyl}-L-alanyl-N-methyl-L-alanyl-N′—[(S)-{[16,20-difluoro-2,3,4,5-tetrahydro-12H-13,17-(azeno)-11,7-(metheno)-1,6,12,14-benzodioxadiazacyclononadecin-9-yl]methyl} (methyl)oxido-lambda⁶-sulfanylidene]-L-aspartamide (Compound 13)

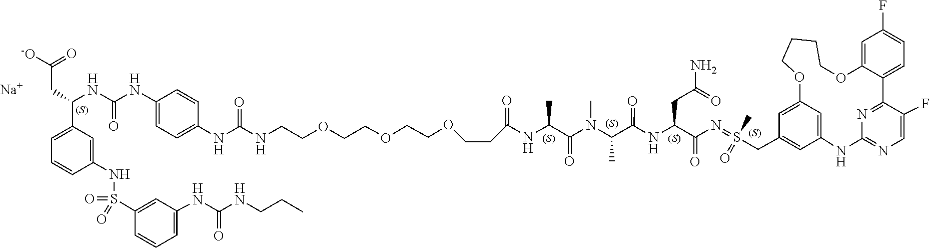

Compound 13 is the epimer of Compound 8 and was synthesized in analogy to Compound 8 using the enantiomeric Building block 2 instead of Building block 1. Yield: 10 mg (100% purity, 70% yield). LC-MS (Method 4): Rₜ=3.07 min; MS (ESIpos): m/z=1514 [M+H]⁺.

Example C1-4: Preparation of Disodium 1-[(2S)-2-(carboxylatomethyl)-17-{4-[({(1S)-2-carboxylato-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (Compound 14)

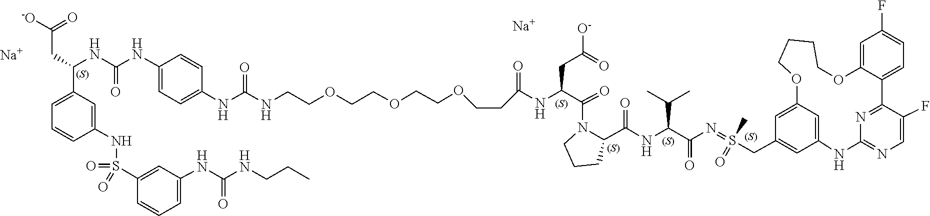

Compound 14 is the epimer of Compound 3 and was synthesized in analogy to Compound 3 using the enantiomeric Building block 2 instead of Building block 1. Yield: 9.5 mg (95% purity, 49% yield over 2 steps). LC-MS (Method 4): Rₜ=3.27 min; MS (ESIpos): m/z=1555 [M+H]⁺.

Example C15: Preparation of N-{2-[{2-[{2-[({4-[({(1R)-2-carboxy-1-[3-({3-[(propylcarbamoyl)amino]benzene-1-sulfonyl}amino)phenyl]ethyl}carbamoyl)amino]phenyl}carbamoyl)amino]ethyl}(methyl)amino]ethyl}(methyl)amino]ethyl}-N-methylglycyl-L-asparaginyl-L-prolyl-N—[(S or R*)—{[3,21-difluoro-13-oxa-5,7,19,26-tetraazatetracyclo[18.3.1.1^{2,6}.1^{8,12}]hexacosa-1(24),2(26),3,5,8(25),9,11,20,22-nonaen-10-yl]methyl}(methyl)oxo-lambda⁶-sulfanylidene]-L-valinamide (Compound 15) *single stereoisomer, absolute stereochemistry unassigned

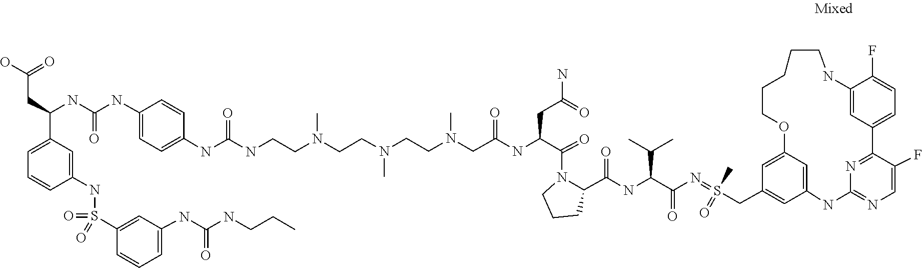 [SMILES: CCCNC(=O)Nc1cccc(S(=O)(=O)Nc2cccc([C@@H](CC(=O)O)NC(=O)Nc3ccc(NC(=O)NCCN(C)CCN(C)CCN(C)CC(=O)N[C@@H](CC(N)=O)C(=O)N4CCC[C@H]4C(=O)N[C@H](C(=O)N=[S@@](C)(=O)Cc4cc5cc(c4)OCCCCCNc4cc(ccc4F)-c4nc(ncc4F)N5)C(C)C)cc3)c2)c1]

The starting material trifluoroacetic acid-N-{2-[{2-[(2-aminoethyl)(methyl) amino]ethyl} (methyl)amino]ethyl}-N-methylglycyl-L-asparaginyl-L-prolyl-N—[(S or R*)—{[3,21-difluoro-13-oxa-5,7,19,26-tetraazatetracyclo[18.3.1.1^{2,6}.1^{8,12}]hexacosa-1(24),2(26),3,5,8(25),9,11,20,22-nonaen-10-yl]methyl}(methyl)oxo-lambda⁶-sulfanylidene]-L-valinamide (1/1) (Intermediate 35) (10.0 mg, 8.88 μmol) was dissolved in DMF (4.0 mL). (3R)-3-{[(4-{[(4-nitrophenoxy)carbonyl] amino}phenyl) carbamoyl]amino}-3-[3-({3-[(propylcarbamoyl) amino] benzene-1-sulfonyl}amino) phenyl] propanoic acid (Building block 1) (7.03 mg, 9.77 μmol) and DIEA (31 μl, 180 μmol) were added. The reaction was stirred for 30 min at RT and then evaporated in vacuo. The crude residue was separated by prep. HPLC to give Compound 15) (10.2 mg, 97% purity, 70% yield) as a yellow foam. LC-MS (Method 3): Rₜ=3.39 min; MS (ESIpos): m/z=1592 [M+H]⁺. ¹H NMR (600 MHz, DMSO-d₆) δ ppm 0.796-0.903 (m, 8H) 0.838-0.868 (m, 1H) 1.385-1.470 (m, 2H) 1.518-1.634 (m, 2H) 1.756-1.828 (m, 4H) 1.828-1.913 (m, 3H) 1.940-1.991 (m, 1H) 2.042-2.199 (m, 1H) 2.291 (br d, J=6.06 Hz, 1H) 2.309 (s, 2H) 2.357-2.477 (m, 2H) 2.542 (s, 5H) 2.545 (s, 1H) 2.592-2.679 (m, 3H) 2.730-2.778 (m, 3H) 2.788-2.845 (m, 4H) 2.845-2.893 (m, 3H) 3.003-3.069 (m, 2H) 3.151-3.193 (m, 2H) 3.193-3.231 (m, 4H) 3.237 (s, 2H) 3.279-3.357 (m, 3H) 3.435-3.474 (m, 3H) 3.579-3.645 (m, 10H) 3.813-3.898 (m, 4H) 3.967-4.027 (m, 3H) 4.119 (br dd, J=9.00, 5.48 Hz, 2H) 4.461-4.499 (m, 1H) 4.776 (br d, J=3.91 Hz, 2H) 4.932-4.974 (m, 1H) 4.974-5.023 (m, 1H) 6.265-6.328 (m, 1H) 6.492-6.567 (m, 1H) 6.618 (s, 1H) 6.710 (br d, J=7.82 Hz, 1H) 6.804 (s, 1H) 6.858-6.913 (m, 1H) 6.979 (br s, 1H) 6.992 (s, 1H) 7.137-7.157 (m, 1H) 7.165 (s, 1H) 7.173-7.204 (m, 1H) 7.204-7.225 (m, 1H) 7.225-7.239 (m, 1H) 7.243 (s, 3H) 7.260-7.298 (m, 1H) 7.411 (d, J=7.93 Hz, 1H) 7.482 (br s, 1H) 7.548 (d, J=8.26 Hz, 1H) 7.796 (br d, J=9.00 Hz, 1H) 8.047-8.086 (m, 1H) 8.334-8.385 (m, 1H) 8.441 (s, 1H) 8.609 (d, J=3.91 Hz, 1H) 8.711 (s, 1H) 8.831 (s, 1H) 8.872-8.970 (m, 1H) 9.783 (s, 1H) 10.262 (s, 1H) 12.051-12.619 (m, 1H).

Example C16: Preparation of N-{2-[{2-[{2-[({4-[({(1S)-2-carboxy-1-[3-({3-[(propylcarbamoyl)amino]benzene-1-sulfonyl}amino)phenyl]ethyl}carbamoyl)amino]phenyl}carbamoyl)amino]ethyl}(methyl)amino]ethyl}(methyl)amino]ethyl}-N-methylglycyl-L-asparaginyl-L-prolyl-N—[(S or R*)—{[3,21-difluoro-13-oxa-5,7,19,26-tetraazatetracyclo[18.3.1.1^{2,6}.1^{8,12}]hexacosa-1(24),2(26),3,5,8(25),9,11,20,22-nonaen-10-yl]methyl}(methyl)oxo-lambda⁶-sulfanylidene]-L-valinamide (Compound 16) *single stereoisomer, absolute stereochemistry unassigned

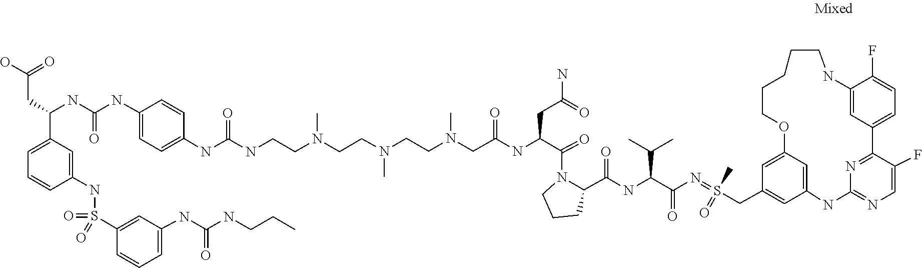 [SMILES: CCCNC(=O)Nc1cccc(S(=O)(=O)Nc2cccc([C@H](CC(=O)O)NC(=O)Nc3ccc(NC(=O)NCCN(C)CCN(C)CCN(C)CC(=O)N[C@@H](CC(N)=O)C(=O)N4CCC[C@H]4C(=O)N[C@H](C(=O)N=[S@@](C)(=O)Cc4cc5cc(c4)OCCCCCNc4cc(ccc4F)-c4nc(ncc4F)N5)C(C)C)cc3)c2)c1]

Trifluoroacetic acid-N-{2-[{2-[(2-aminoethyl)(methyl)amino]ethyl}(methyl)amino]ethyl}-N-methylglycyl-L-asparaginyl-L-prolyl-N—[(S or R*)—{[3,21-difluoro-13-oxa-5,7,19,26-tetraazatetracyclo[18.3.1.1^{2,6}.1^{8,12}]hexacosa-1(24),2(26),3,5,8(25),9,11,20,22-nonaen-10-yl]methyl}(methyl)oxo-lambda⁶-sulfanylidene]-L-valinamide (1/1) (Intermediate 35) (10.0 mg, 8.88 μmol) was dissolved in DMF (4.0 mL). (3S)-3-{[(4-{[(4-nitrophenoxy)carbonyl]amino}phenyl) carbamoyl]amino}-3-[3-({3-[(propylcarbamoyl)amino]benzene-1-sulfonyl}amino) phenyl]propanoic acid (Building block 2) (7.03 mg, 9.77 μmol) and DIEA (31 μl, 180 μmol) were added. The reaction was stirred for 30 min at RT, then the reaction was concentrated in vacuo. The residue was separated twice by prep. HPLC to give Compound 16 (2.10 mg, 100% purity, 15% yield) as a yellow foam. LC-MS (Method 4): Rₜ=2.66 min; MS (ESIpos): m/z=1592 [M+H]⁺.

Example C17: Preparation of N-{2-[{2-[{2-[({4-[({(1R)-2-carboxy-1-[3-({3-[(propylcarbamoyl)amino]benzene-1-sulfonyl}amino)phenyl]ethyl}carbamoyl)amino]phenyl}carbamoyl)amino]ethyl}(methyl)amino]ethyl}(methyl)amino]ethyl}-N-methylglycyl-L-asparaginyl-L-prolyl-N—[(R or S*)—{[3,21-difluoro-13-oxa-5,7,19,26-tetraazatetracyclo[18.3.1.1^{2,6}.1^{8,12}]hexacosa-1(24),2(26),3,5,8(25),9,11,20,22-nonaen-10-yl]methyl}(methyl)oxo-lambda⁶-sulfanylidene]-L-valinamide (Compound 17) *single stereoisomer, absolute stereochemistry unassigned

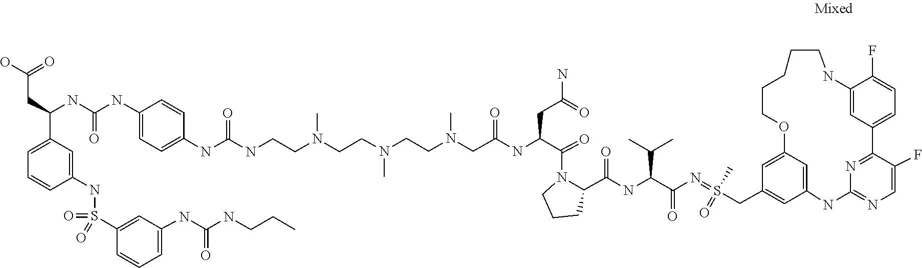 [SMILES: CCCNC(=O)Nc1cccc(S(=O)(=O)Nc2cccc([C@@H](CC(=O)O)NC(=O)Nc3ccc(NC(=O)NCCN(C)CCN(C)CCN(C)CC(=O)N[C@@H](CC(N)=O)C(=O)N4CCC[C@H]4C(=O)N[C@H](C(=O)N=[S@](C)(=O)Cc4cc5cc(c4)OCCCCCNc4cc(ccc4F)-c4nc(ncc4F)N5)C(C)C)cc3)c2)c1]

Trifluoroacetic acid-N-{2-[{2-[(2-aminoethyl)(methyl)amino]ethyl}(methyl) amino]ethyl}-N-methylglycyl-L-asparaginyl-L-prolyl-N—[(R or S*)—{[3,21-difluoro-13-oxa-5,7,19,26-tetraazatetracyclo[18.3.1.1^{2,6}.1^{8,12}]hexacosa-1(24),2(26),3,5,8(25),9,11,20,22-nonaen-10-yl]methyl}(methyl) oxo-lambda⁶-sulfanylidene]-L-valinamide (1/1) (Intermediate 38) (10.0 mg, 8.88 μmol) was dissolved in DMF (4.0 mL). (3R)-3-{[(4-{[(4-nitrophenoxy)carbonyl]amino}phenyl)carbamoyl]amino}-3-[3-({3-[(propylcarbamoyl)amino] benzene-1-sulfonyl}amino)phenyl]propanoic acid (Building block 1) (7.03 mg, 9.77 μmol) and DIEA (31 μl, 180 μmol) were added. The reaction was stirred for 30 min at RT, then the reaction was concentrated in vacuo. The residue was separated by prep. HPLC to give Compound 17 (2.50 mg, 100% purity, 18% yield) as a yellow foam. LC-MS (Method 4): Rₜ=2.66 min; MS (ESIpos): m/z=1592 [M+H]⁺.

Example C18: Preparation of trisodium 1-[(2S)-15-{[N²,N⁶-bis(14-{4-[({(1R)-2-carboxylato-1-[3-({3-[(propylcarbamoyl) amino]benzene-1-sulfonyl}amino)phenyl]ethyl}carbamoyl)amino]anilino}-14-oxo-4,7,10-trioxa-13-azatetradecanan-1-oyl)-L-lysyl]amino}-2-(carboxylatomethyl)-4-oxo-7,10,13-trioxa-3-azapentadecanan-1-oyl]-L-prolyl-N—[(S)-{[16,20-difluoro-2,3,4,5-tetrahydro-12H-17,13-(azeno)-11,7-(metheno)-1,6,12,14-benzodioxadiazacyclo-nonadecin-9-yl]methyl}(methyl)oxo-lambda⁶-sulfanylidene]-L-valinamide (Compound 18)

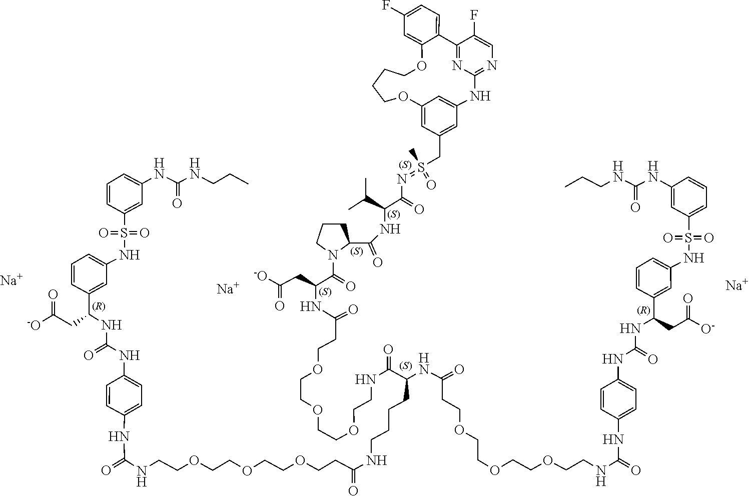

N-(3-{2-[2-(2-{[N²,N⁶-Bis(3-{2-[2-(2-aminoethoxy)ethoxy]ethoxy}propanoyl)-L-lysyl] amino} ethoxy) ethoxy]ethoxy}propanoyl)-L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclo-nonadecin-9-yl] methyl} (methyl)oxo-lambda⁶-sulfanylidene]-L-valinamide trifluoroacetic acid (1/2) (Intermediate 42) (35.0 mg, 20.1 μmol) was dissolved in DMF (10.0 mL). (3R)-3-{[(4-{[(4-Nitrophenoxy) carbonyl]amino}phenyl)carbamoyl]amino}-3-[3-({3-[(propylcarbamoyl)amino]benzene-1-sulfonyl} amino) phenyl]propanoic acid (Building block 1) (29.0 mg, 40.3 μmol) and DIEA (70 μl, 400 μmol) were added. The reaction was stirred for 30 min at RT and then concentrated in vacuo. The residue was separated by prep. HPLC to give the parent compound (18.0 mg, 97% purity, 33% yield) as a yellow foam. LC-MS: Rₜ=4.45 min; MS (ESIpos): m/z=1336 [M+2H]²⁺.

The parent compound (18.0 mg, 6.74 μmol) was dissolved in dioxane/water (1:1, 10 mL). A sodium hydroxide solution (200 μl, 0.10 M, 20 μmol) was added. The solution was lyophilized to give Compound 18 as a trisodium salt (18.0 mg, 100% purity, 98% yield). LC-MS (Method 4): Rₜ=3.41 min; MS (ESIpos): m/z=1336 [M+2H]²⁺. ¹H NMR (600 MHz, DMSO-d₆) δ ppm 0.798-0.859 (m, 1H) 0.804-0.823 (m, 1H) 1.187-1.264 (m, 1H) 1.322-1.396 (m, 1H) 1.420-1.503 (m, 1H) 1.549-1.634 (m, 1H) 1.819-1.891 (m, 1H) 1.897-1.962 (m, 1H) 2.046-2.128 (m, 1H) 2.284 (br t, J=6.55 Hz, 1H) 2.301-2.353 (m, 1H) 2.353-2.404 (m, 1H) 2.522-2.535 (m, 1H) 2.541 (s, 1H) 2.581-2.626 (m, 1H) 2.626-2.681 (m, 1 H) 2.914-2.960 (m, 1H) 2.960-3.010 (m, 1H) 3.150-3.189 (m, 1H) 3.177-3.191 (m, 1H) 3.189-3.235 (m, 1H) 3.368-3.398 (m, 1H) 3.424 (br t, J=5.67 Hz, 1H) 3.440-3.505 (m, 1H) 3.468-3.487 (m, 1H) 3.514 (s, 1H) 3.547-3.600 (m, 1H) 3.671-3.728 (m, 1H) 4.033-4.062 (m, 1H) 4.112-4.151 (m, 1H) 4.172-4.210 (m, 1H) 4.251-4.280 (m, 1H) 4.455-4.497 (m, 1H) 4.690-4.730 (m, 1H) 4.832-4.860 (m, 1H) 4.952-4.987 (m, 1H) 6.226-6.261 (m, 1H) 6.517 (s, 1H) 6.645-6.684 (m, 1H) 6.870 (t, J=8.03 Hz, 1H) 6.941 (br d, J=7.63 Hz, 1H) 7.064 (t, J=7.80 Hz, 1H) 7.134-7.166 (m, 1H) 7.166-7.182 (m, 1H) 7.191 (br d, J=3.13 Hz, 1H) 7.208 (s, 1H) 7.247 (s, 1H) 7.262 (s, 1H) 7.295 (t, J=8.02 Hz, 1H) 7.314-7.344 (m, 1H) 7.374-7.405 (m, 1H) 7.539 (br s, 1H) 7.796-7.822 (m, 1H) 7.800-7.879 (m, 1H) 7.945 (br t, J=5.77 Hz, 1H) 7.997 (br s, 1H) 8.002-8.016 (m, 1H) 8.132-8.158 (m, 1H) 8.187-8.251 (m, 1H) 8.467-8.497 (m, 1H) 8.541-8.662 (m, 1H) 8.674 (s, 1H) 8.811-8.835 (m, 1H) 9.770 (br s, 1H) 9.852-10.059 (m, 1H) 11.015-11.222 (m, 1H).

Example C19: Preparation of trisodium 1-[(2S)-15-{[N²,N⁶-bis(14-{4-[({(1S)-2-carboxylato-1-[3-({3-[(propylcarbamoyl) amino] benzene-1-sulfonyl}amino)phenyl]ethyl}carbamoyl)amino]anilino}-14-oxo-4,7,10-trioxa-13-azatetradecanan-1-oyl)-L-lysyl]amino}-2-(carboxylatomethyl)-4-oxo-7,10,13-trioxa-3-azapentadecanan-1-oyl]-L-prolyl-N—[(S)-{[16,20-difluoro-2,3,4,5-tetrahydro-12H-17,13-(azeno)-11,7-(metheno)-1,6,12,14-benzodioxadiazacyclononadecin-9-yl]methyl}(methyl)oxo-lambda⁶-sulfanylidene]-L-valinamide (Compound 19)

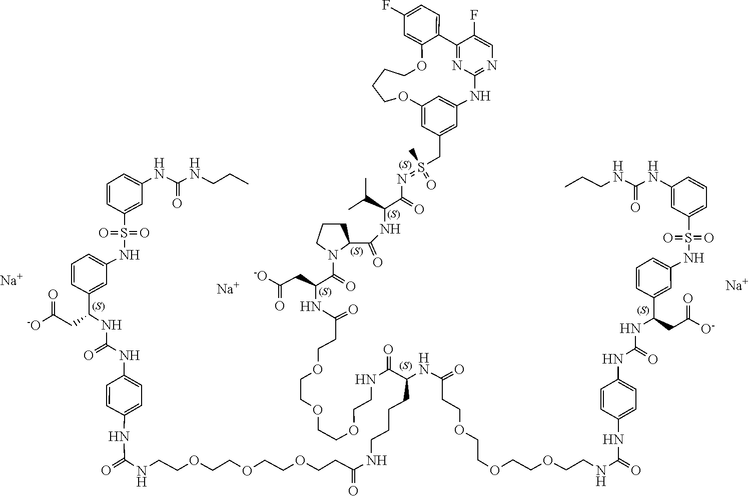

(3-{2-[2-(2-{[N²,N⁶-Bis(3-{2-[2-(2-aminoethoxy)ethoxy]ethoxy}propanoyl)-L-lysyl] amino}ethoxy)ethoxy]ethoxy}propanoyl)-L-alpha-aspartyl-L-prolyl-N—[(S)-{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide trifluoroacetic acid (1/2) (Intermediate 42) (15.0 mg, 8.63 μmol) was dissolved in DMF (5.0 mL). (3S)-3-{[(4-{[(4-nitrophenoxy) carbonyl] amino} phenyl)carbamoyl]amino}-3-[3-({3-[(propylcarbamoyl) amino]benzene-1-sulfonyl} amino) phenyl]propanoic acid (Building block 2) (12.4 mg, 17.3 μmol) and DIEA (30 μl, 170 μmol) were added. The reaction was stirred for 30 min at RT and then concentrated in vacuo. The residue was separated by prep. HPLC to give the parent compound (9.00 mg, 100% purity, 39% yield) as a yellow foam. LC-MS: Rₜ=4.45 min; MS (ESIpos): m/z=1335 [M+2H]²⁺.

The parent compound (9.00 mg, 3.37 μmol) was dissolved in dioxane/water (1:1, 5.0 mL). A sodium hydroxide solution (100 μl, 0.10 M, 10 μmol) was added. The solution was lyophilized to give Compound 19 as the trisodium salt. (9.00 mg, 100% purity, 98% yield) LC-MS (Method 4): Rₜ=3.41 min; MS (ESIpos): m/z=1335 [M+2H]²⁺. ¹H NMR (600 MHz, DMSO-d₆) δ ppm 0.069 (s, 1H) 0.806-0.864 (m, 3H) 0.816-0.842 (m, 1H) 1.168-1.274 (m, 1H) 1.310-1.411 (m, 1H) 1.321-1.405 (m, 1H) 1.418-1.499 (m, 1H) 1.535-1.630 (m, 1H) 1.811-1.938 (m, 2H) 2.062-2.110 (m, 1H) 2.270-2.302 (m, 1H) 2.302-2.349 (m, 1H) 2.349-2.425 (m, 1H) 2.541 (s, 5H) 2.584-2.635 (m, 1H) 2.658-2.732 (m, 1H) 2.918-2.970 (m, 1H) 2.970-3.020 (m, 1H) 3.178 (s, 1H) 3.192-3.238 (m, 2H) 3.361-3.395 (m, 2H) 3.426 (br t, J=5.48 Hz, 1H) 3.441-3.503 (m, 6H) 3.468-3.486 (m, 1H) 3.514 (s, 2H) 3.537-3.619 (m, 3H) 3.556-3.592 (m, 1H) 3.623-3.669 (m, 1H) 3.828 (br s, 1H) 3.963-4.021 (m, 1H) 4.051 (dd, J=8.71, 5.58 Hz, 1H) 4.108-4.148 (m, 1H) 4.167-4.221 (m, 1H) 4.250-4.279 (m, 1H) 4.446-4.497 (m, 1H) 4.712 (s, 1H) 4.839 (t, J=2.54 Hz, 1H) 4.850-4.907 (m, 1H) 4.954-4.992 (m, 1H) 6.130-6.161 (m, 1H) 6.515 (s, 1H) 6.654-6.674 (m, 1H) 6.674-6.706 (m, 1H) 6.871 (t, J=8.10 Hz, 1H) 6.946 (br d, J=7.24 Hz, 1H) 7.077 (t, J=7.84 Hz, 1H) 7.148 (dd, J=11.74, 2.35 Hz, 1H) 7.175-7.188 (m, 1H) 7.190 (br s, 1H) 7.197 (br d, J=1.56 Hz, 1H) 7.203-7.215 (m, 1H) 7.236-7.248 (m, 1H) 7.236-7.259 (m, 1H) 7.292 (t, J=8.01 Hz, 1H) 7.393 (t, J=7.89 Hz, 1H) 7.596-7.626 (m, 1H) 7.705-7.745 (m, 1H) 7.745-7.779 (m, 1H) 7.779-7.808 (m, 1H) 7.907-7.932 (m, 1H) 7.945-7.977 (m, 1H) 7.977-8.009 (m, 1H) 8.009-8.064 (m, 1H) 8.275 (br d, J=8.22 Hz, 1H) 8.373-8.408 (m, 1H) 8.662 (d, J=1.56 Hz, 1H) 8.735-8.768 (m, 1H) 9.722 (s, 1H) 9.865-10.080 (m, 1H) 10.555-11.042 (m, 1H) 12.023 (s, 1H).

Example C20: Preparation of (16S)-1-{4-[({(1R)-2-carboxy-1-[3-({3-[(propylcarbamoyl)amino]benzene-1-sulfonyl}amino)phenyl]ethyl}carbamoyl)amino]anilino}-16-[(2S)-2-{[(2S)-1-{[(R*)—{[5,23-difluoro-8,13-dioxa-19,21,24-triazatetracyclo[18.3.1.1^{14,18}.0^{2,7}]pentacosa-1(24),2,4,6,14(25),15,17,20,22-nonaen-16-yl]methyl} (methyl)oxo-lambda⁶-sulfanylidene]amino}-3-methyl-1-oxobutan-2-yl]carbamoyl}pyrrolidine-1-carbonyl]-N,N,N-trimethyl-1,14,18-trioxo-5,8,11,19-tetraoxa-2,15-diazadocosan-22-aminium trifluoroacetate (Compound 20)

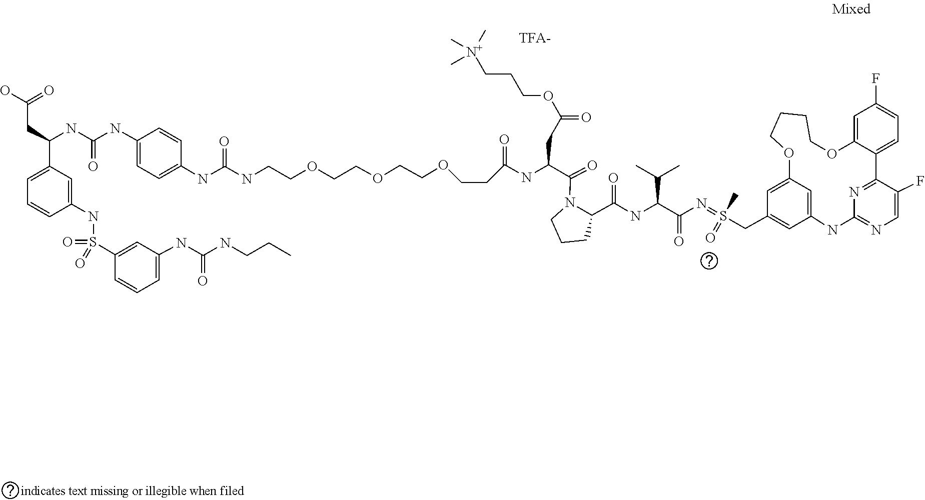 [SMILES: CCCNC(=O)Nc1cccc(S(=O)(=O)Nc2cccc([C@@H](CC(=O)O)NC(=O)Nc3ccc(NC(=O)NCCOCCOCCOCCC(=O)N[C@@H](CC(=O)OCCC[N+](C)(C)C)C(=O)N4CCC[C@H]4C(=O)N[C@H](C(=O)N=[S@@](C)(=O)Cc4cc5cc(c4)OCCCCOc4cc(F)ccc4-c4nc(ncc4F)N5)C(C)C)cc3)c2)c1]

(14S)-1-Amino-14-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-N,N,N-trimethyl-12,16-dioxo-3,6,9,17-tetraoxa-13-azaicosan-20-aminium trifluoroacetate trifluoroacetic acid (Intermediate 49) (26.0 mg, 20.0 μmol) was dissolved in DMF (10 mL). Intermediate B1 (14.4 mg, 20.0 μmol) and DIEA (35 μl, 200 μmol; CAS-RN:[7087-68-5]) were added. The reaction was stirred for 30 minutes at RT. The reaction was evaporated to dryness and the residue was separated by prep. HPLC to give Compound 20 (23.2 mg, 98% purity, 64% yield) as a colourless foam. LC-MS (Method 3): Rt=3.64 min; MS (ESIpos): m/z=1655 [M+H]⁺; ¹H-NMR (600 MHz, DMSO-d₆) δ[ppm]: 0.831 (2.96), 0.836 (3.21), 0.844 (4.51), 0.848 (3.18), 0.856 (5.57), 0.869 (2.55), 1.222 (0.48), 1.233 (0.50), 1.411 (0.85), 1.423 (1.49), 1.435 (1.47), 1.447 (0.79), 1.817 (0.47), 1.868 (1.48), 2.010 (0.48), 2.020 (0.57), 2.028 (0.56), 2.037 (0.45), 2.087 (0.41), 2.337 (0.60), 2.347 (1.08), 2.357 (0.57), 2.519 (0.65), 2.631 (1.33), 2.643 (1.23), 2.750 (0.47), 2.761 (0.47), 2.776 (0.40), 3.017 (0.77), 3.029 (1.84), 3.039 (16.00), 3.050 (1.45), 3.187 (5.17), 3.215 (0.96), 3.223 (0.95), 3.332 (0.80), 3.346 (0.75), 3.360 (0.74), 3.421 (1.16), 3.430 (1.91), 3.439 (0.92), 3.468 (1.39), 3.471 (1.84), 3.481 (1.97), 3.485 (1.25), 3.488 (1.10), 3.508 (0.80), 3.575 (1.15), 3.585 (2.24), 3.596 (1.37), 4.036 (0.98), 4.045 (1.57), 4.054 (1.76), 4.060 (1.25), 4.064 (1.09), 4.131 (0.84), 4.273 (1.03), 4.456 (0.51), 4.462 (0.49), 4.471 (0.51), 4.475 (0.47), 4.717 (1.81), 4.983 (0.69), 4.994 (0.83), 5.006 (0.64), 6.091 (0.41), 6.265 (0.60), 6.510 (1.11), 6.651 (0.67), 6.668 (1.48), 6.878 (0.64), 6.882 (0.67), 6.892 (0.85), 6.896 (0.89), 6.907 (0.65), 6.981 (0.67), 6.994 (0.76), 7.143 (1.73), 7.147 (1.88), 7.156 (1.41), 7.169 (1.01), 7.210 (7.66), 7.240 (0.58), 7.242 (0.45), 7.253 (1.16), 7.267 (1.03), 7.280 (1.38), 7.293 (0.57), 7.399 (1.01), 7.410 (0.72), 7.742 (0.64), 7.757 (0.60), 8.002 (0.97), 8.062 (0.93), 8.065 (1.50), 8.069 (0.86), 8.353 (1.22), 8.378 (1.23), 8.394 (0.72), 8.408 (0.65), 8.677 (1.64), 8.681 (1.48), 8.804 (1.36), 9.732 (1.41), 10.269 (1.94).

Example C21: Preparation of (3R)-3-[({4-[({(10S)-10-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-2-methyl-8,12-dioxo-7,15,18,21-tetraoxa-2,11-diazatricosan-23-yl}carbamoyl)amino]phenyl}carbamoyl)amino]-3-[3-({3-[(propylcarbamoyl)amino]benzene-1-sulfonyl}amino)phenyl]propanoic acid (Compound 21)

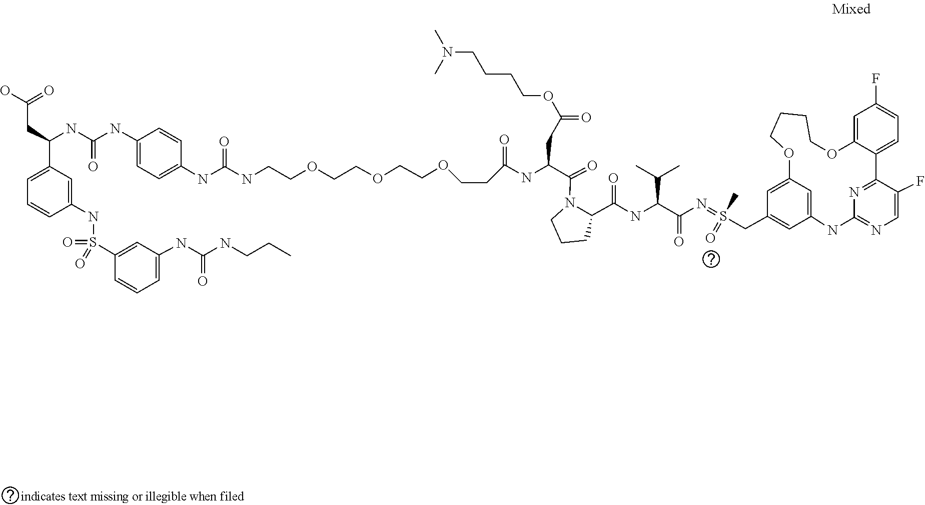 [SMILES: CCCNC(=O)Nc1cccc(S(=O)(=O)Nc2cccc([C@@H](CC(=O)O)NC(=O)Nc3ccc(NC(=O)NCCOCCOCCOCCC(=O)N[C@@H](CC(=O)OCCCCN(C)C)C(=O)N4CCC[C@H]4C(=O)N[C@H](C(=O)N=[S@@](C)(=O)Cc4cc5cc(c4)OCCCCOc4cc(F)ccc4-c4nc(ncc4F)N5)C(C)C)cc3)c2)c1]

To a solution of trifluoroacetic acid 4-(dimethylamino)butyl (14S)-1-amino-14-[(2S)-2-{[(2S)-1-{[(R*)—{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-12-oxo-3,6,9-trioxa-13-azahexadecan-16-oate (Intermediate 56) (23.0 mg, 96% purity, 18.6 μmol) in DMF (8.0 mL) were added (3R)-3-{[(4-{[(4-nitrophenoxy)carbonyl]amino} phenyl)carbamoyl] amino}-3-[3-({3-[(propylcarbamoyl)amino]benzene-1-sulfonyl}amino)phenyl]propanoic acid (Building block 1) (21.1 mg, 95% purity, 27.9 μmol) and (5.0 eq) N,N-diisopropylethylamine (16 μl, 93 μmol; CAS-RN:[7087-68-5]), then the reaction was stirred at RT for 1 h and concentrated in vacuo. The residue was purified by prep. HPLC and lyophilized to give the title compound as an amorphous residue (20.0 mg, 88% purity, 57% yield). LC-MS (Method 3): Rₜ=3.66 min; MS (ESIpos): m/z=1653 [M+H]⁺.

Example C22: Preparation of N-{2-[{2-[{2-[({4-[({(1R)-2-carboxy-1-[3-({3-[(propylcarbamoyl)amino]benzene-1-sulfonyl}amino)phenyl]ethyl}carbamoyl)amino]phenyl}carbamoyl)amino]ethyl}(methyl)amino]ethyl}(methyl)amino]ethyl}-N-methylglycyl-L-asparaginyl-L-prolyl-N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (Compound 22)

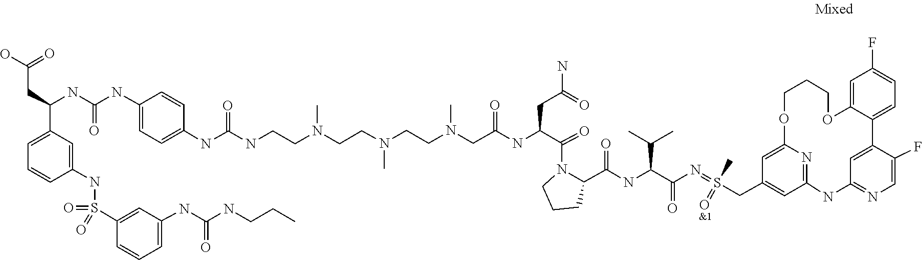 [SMILES: CCCNC(=O)Nc1cccc(S(=O)(=O)Nc2cccc([C@@H](CC(=O)O)NC(=O)Nc3ccc(NC(=O)NCCN(C)CCN(C)CCN(C)CC(=O)N[C@@H](CC(N)=O)C(=O)N4CCC[C@H]4C(=O)N[C@H](C(=O)N=[S@@](C)(=O)Cc4cc5nc(c4)OCCCOc4cc(F)ccc4-c4cc(ncc4F)N5)C(C)C)cc3)c2)c1]

Trifluoroacetic acid N-{2-[{2-[(2-aminoethyl)(methyl)amino]ethyl}(methyl)amino]ethyl}-N-methylglycyl-L-asparaginyl-L-prolyl-N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (1/1) (12.0 mg, 10.9 μmol) (Intermediate 60) was dissolved in DMF (5.0 mL). (3R)-3-{[(4-{[(4-nitrophenoxy)carbonyl]amino}phenyl) carbamoyl]amino}-3-[3-({3-[(propylcarbamoyl)amino]benzene-1-sulfonyl}amino)phenyl] propanoic acid (7.86 mg, 10.9 μmol) (Building block 1) and 20 eq DIEA (38 μl, 220 μmol; CAS-RN:[11-5]) were added. The reaction was stirred for 30 minutes at RT and then evaporated to dryness. The residue was separated by prep. HPLC to give Compound 22 (8.55 mg, 100% purity, 50% yield) as a colorless foam. LC-MS (Method 3): Rₜ=3.21 min; MS (ESIpos): m/z=1565 [M+H]⁺. ¹H-NMR (600 MHz, DMSO-d₆) δ[ppm]: 0.069 (1.44), 0.821 (3.14), 0.828 (3.78), 0.832 (4.14), 0.839 (6.14), 0.843 (6.35), 0.850 (3.83), 0.856 (11.20), 0.862 (3.63), 0.868 (6.28), 0.872 (3.40), 1.236 (0.43), 1.411 (1.81), 1.423 (3.24), 1.434 (3.22), 1.447 (1.67), 1.859 (0.76), 1.937 (0.74), 2.080 (1.70), 2.090 (1.96), 2.123 (0.75), 2.305 (3.43), 2.386 (1.20), 2.412 (0.89), 2.425 (1.16), 2.520 (1.89), 2.631 (3.50), 2.643 (3.00), 2.753 (2.83), 2.804 (2.27), 2.863 (3.23), 3.016 (1.35), 3.027 (2.88), 3.038 (2.90), 3.049 (1.36), 3.188 (6.71), 3.240 (0.99), 3.261 (1.02), 3.304 (1.48), 3.449 (1.90), 3.628 (1.74), 3.681 (1.70), 3.725 (1.84), 3.852 (1.95), 4.088 (0.96), 4.098 (1.58), 4.114 (2.86), 4.123 (3.14), 4.516 (1.60), 4.758 (1.18), 4.773 (1.79), 4.797 (0.55), 4.866 (0.75), 4.889 (0.66), 4.947 (0.64), 4.978 (0.72), 4.990 (1.28), 5.003 (1.27), 5.015 (0.53), 6.261 (2.42), 6.277 (1.67), 6.315 (2.00), 6.487 (0.71), 6.557 (1.75), 6.585 (1.79), 6.692 (1.32), 6.706 (1.19), 6.878 (1.41), 6.890 (2.17), 6.903 (1.40), 6.917 (0.74), 6.979 (2.19), 6.992 (2.55), 7.074 (1.33), 7.090 (1.32), 7.138 (1.57), 7.151 (2.67), 7.164 (3.73), 7.242 (16.00), 7.264 (1.94), 7.277 (2.72), 7.291 (1.12), 7.406 (1.67), 7.419 (1.40), 7.468 (1.02), 7.577 (0.95), 7.789 (0.65), 7.804 (0.63), 7.861 (0.65), 7.875 (0.60), 8.064 (3.12), 8.323 (2.76), 8.431 (2.23), 8.684 (2.15), 8.692 (2.81), 8.702 (1.24), 8.815 (3.11), 8.926 (0.54), 9.624 (1.55), 9.681 (1.60), 10.262 (4.04).

Example C23: Preparation of N-{2-[{2-[{2-[({4-[({(1S)-2-carboxy-1-[3-({3-[(propylcarbamoyl)amino] benzene-1-sulfonyl}amino)phenyl]ethyl}carbamoyl)amino]phenyl}carbamoyl)amino]ethyl} (methyl)amino]ethyl}(methyl)amino]ethyl}-N-methylglycyl-L-asparaginyl-L-prolyl-N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (Compound 23)

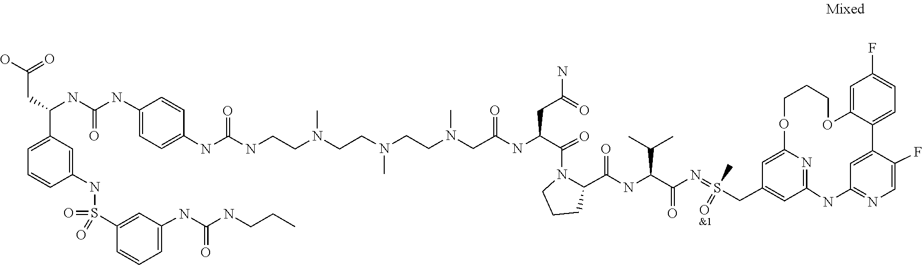 [SMILES: CCCNC(=O)Nc1cccc(S(=O)(=O)Nc2cccc([C@H](CC(=O)O)NC(=O)Nc3ccc(NC(=O)NCCN(C)CCN(C)CCN(C)CC(=O)N[C@@H](CC(N)=O)C(=O)N4CCC[C@H]4C(=O)N[C@H](C(=O)N=[S@@](C)(=O)Cc4cc5nc(c4)OCCCOc4cc(F)ccc4-c4cc(ncc4F)N5)C(C)C)cc3)c2)c1]

Trifluoroacetic acid N-{2-[{2-[(2-aminoethyl)(methyl)amino]ethyl}(methyl) amino]ethyl}-N-methylglycyl-L-asparaginyl-L-prolyl-N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (1/1) (10.0 mg, 9.10 μmol) (1/1) (Intermediate 60) was dissolved in DMF (5.0 mL). (3S)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (6.55 mg, 9.10 μmol) (Building block 2) and 20 eq DIEA (32 μl, 180 μmol; CAS-RN:[7087-68-5]) were added. The reaction was stirred for 30 minutes at RT and then evaporated to dryness. The residue was separated by prep. HPLC to give Compound 23 (7.50 mg, 100% purity, 53% yield) as a colorless foam. LC-MS (Method 3): Rₜ=3.21 min; MS (ESIpos): m/z=1565 [M+H]⁺. ¹H-NMR (600 MHz, DMSO-d₆) δ[ppm]: 0.069 (0.65), 0.822 (3.48), 0.828 (3.98), 0.833 (4.46), 0.839 (6.31), 0.843 (6.85), 0.851 (4.17), 0.856 (11.48), 0.862 (3.64), 0.868 (6.24), 0.873 (3.34), 0.894 (0.65), 1.236 (0.52), 1.399 (0.60), 1.411 (1.91), 1.423 (3.23), 1.434 (3.16), 1.447 (1.72), 1.459 (0.48), 1.756 (0.42), 1.889 (0.96), 2.080 (1.84), 2.090 (2.05), 2.112 (1.24), 2.123 (0.92), 2.135 (0.68), 2.316 (4.65), 2.386 (1.57), 2.413 (1.50), 2.425 (1.71), 2.438 (1.41), 2.517 (2.39), 2.520 (2.36), 2.523 (2.17), 2.614 (0.80), 2.631 (2.96), 2.642 (2.25), 2.731 (0.81), 2.757 (4.28), 2.819 (2.00), 2.867 (5.23), 2.891 (0.88), 3.016 (1.13), 3.027 (2.27), 3.038 (2.25), 3.049 (0.99), 3.188 (6.47), 3.214 (1.89), 3.240 (0.74), 3.261 (0.84), 3.315 (1.39), 3.451 (1.54), 3.628 (0.72), 3.729 (0.53), 3.856 (1.68), 4.089 (1.24), 4.099 (1.82), 4.114 (2.95), 4.123 (3.16), 4.509 (1.74), 4.736 (0.63), 4.758 (1.16), 4.774 (1.68), 4.797 (0.53), 4.866 (0.64), 4.889 (0.57), 4.962 (0.56), 4.979 (0.67), 4.991 (1.16), 5.004 (1.05), 6.261 (2.16), 6.280 (0.92), 6.290 (1.33), 6.299 (0.72), 6.316 (1.75), 6.518 (0.87), 6.558 (1.50), 6.585 (1.60), 6.699 (1.20), 6.713 (1.05), 6.880 (1.47), 6.890 (1.98), 6.904 (1.25), 6.918 (0.64), 6.979 (2.07), 6.992 (2.20), 7.074 (1.22), 7.089 (1.22), 7.137 (1.64), 7.151 (2.62), 7.163 (3.33), 7.243 (16.00), 7.264 (1.80), 7.277 (2.43), 7.290 (0.86), 7.407 (1.32), 7.420 (1.09), 7.472 (0.78), 7.576 (0.74), 7.788 (0.53), 7.803 (0.44), 7.861 (0.45), 7.875 (0.40), 8.066 (2.85), 8.323 (2.46), 8.437 (2.00), 8.684 (1.36), 8.693 (2.05), 8.703 (1.79), 8.825 (2.73), 8.923 (0.53), 9.624 (1.24), 9.681 (1.39), 10.262 (3.70).

Example C24: Preparation of N-{2-[{2-[{2-[({4-[({(1R)-2-carboxy-1-[3-({3-[(propylcarbamoyl)amino]benzene-1-sulfonyl}amino)phenyl]ethyl}carbamoyl)amino]phenyl}carbamoyl)amino]ethyl}(methyl)amino]ethyl}(methyl)amino]ethyl}-N-methylglycyl-L-asparaginyl-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (Compound 24)

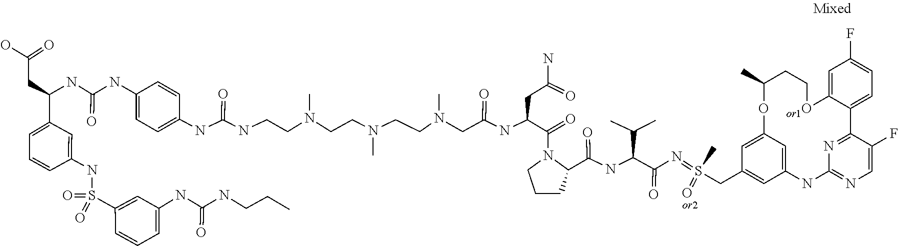

To a solution of trifluoroacetic acid N-{2-[{2-[(2-aminoethyl)(methyl)amino]ethyl}(methyl)amino]ethyl}-N-methylglycyl-L-asparaginyl-L-prolyl-N—[(R*)—{[(4R*)-15,19-difluoro-4-methyl-3,4-dihydro-2H, 11H-12,16-(azeno)-10,6-(metheno)-1,5,11,13-benzodioxadiazacyclooctadecin-8-yl]methyl}(methyl)oxo-lambda6-sulfanylidene]-L-valinamide (1/1) (10.0 mg, 8.98 μmol) (Intermediate 62) in DMF (5.0 mL) was added (3R)-3-{[(4-{[(4-nitrophenoxy)carbonyl]amino}phenyl) carbamoyl]amino}-3-[3-({3-[(propylcarbamoyl)amino]benzene-1-sulfonyl}amino) phenyl]propanoic acid (9.70 mg, 13.5 μmol) (Building block 1) and N,N-diisopropylethylamine (7.8 μl, 45 μmol; CAS-RN.[7087-68-5]), then the reaction was stirred at RT for 2 h. The reaction was concentrated in vacuo. The residue was purified by prep. HPLC, then lyophilized to give Compound 24 (5.00 mg, 100% purity, 35% yield) as an amorphous residue. LC-MS (Method 3): Rₜ=3.38 min; MS (ESIpos): m/z=1578 [M+H]⁺. ¹H-NMR (600 MHz, DMSO-d₆) δ[ppm]: −0.100 (1.11), −0.061 (0.48), 0.067 (0.79), 0.097 (1.13), 0.828 (5.85), 0.840 (11.04), 0.843 (9.11), 0.852 (7.12), 0.856 (13.08), 0.868 (5.98), 0.879 (1.04), 0.890 (0.91), 1.236 (0.52), 1.399 (0.59), 1.411 (2.15), 1.427 (6.10), 1.435 (7.75), 1.447 (2.29), 1.720 (0.93), 1.893 (1.27), 1.978 (0.68), 2.094 (0.70), 2.105 (1.02), 2.115 (0.97), 2.300 (3.44), 2.328 (1.25), 2.386 (1.54), 2.403 (0.86), 2.415 (0.93), 2.424 (1.09), 2.516 (2.45), 2.519 (2.42), 2.522 (2.29), 2.614 (1.45), 2.631 (3.78), 2.643 (3.10), 2.751 (2.76), 2.797 (2.56), 2.863 (3.08), 3.016 (1.56), 3.028 (3.29), 3.038 (3.24), 3.049 (1.54), 3.110 (0.59), 3.165 (2.38), 3.194 (12.62), 3.301 (1.81), 3.853 (1.43), 4.085 (1.11), 4.100 (2.22), 4.109 (1.68), 4.115 (1.45), 4.124 (1.22), 4.371 (0.88), 4.473 (1.97), 4.712 (3.76), 4.946 (0.86), 4.991 (1.36), 5.003 (1.36), 6.263 (1.75), 6.485 (2.61), 6.683 (1.34), 6.697 (1.25), 6.734 (2.76), 6.877 (1.43), 6.889 (1.56), 6.914 (1.02), 6.931 (1.63), 6.942 (0.91), 6.978 (2.38), 6.990 (2.52), 7.137 (1.77), 7.150 (2.86), 7.164 (4.19), 7.241 (16.00), 7.257 (1.18), 7.265 (2.40), 7.278 (2.99), 7.291 (1.22), 7.353 (1.41), 7.370 (1.41), 7.405 (1.75), 7.419 (1.45), 7.477 (1.50), 7.610 (0.63), 7.621 (1.02), 7.629 (1.02), 7.643 (0.63), 7.779 (1.41), 7.793 (1.31), 8.062 (3.35), 8.424 (2.40), 8.673 (3.99), 8.678 (3.20), 8.705 (2.67), 8.801 (3.40), 9.745 (3.17), 10.261 (4.35).

Example C25: Preparation of (3R)-3-[({4-[({(20S,40S)-42-amino-20-[(17-{4-[({(1R)-2-carboxy-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecan-1-yl)carbamoyl]-40-[(2S)-2-{[(2S)-1-{[(S)-{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-30,33,36-trimethyl-12,17,22,26,38,42-hexaoxo-3,6,9-trioxa-13,16,21,27,30,33,36,39-octaazadotetracontan-1-yl}carbamoyl)amino]phenyl}carbamoyl)amino]-3-[3-({3-[(propylcarbamoyl)amino]benzene-1-sulfonyl}amino)phenyl]propanoic acid (Compound 25)

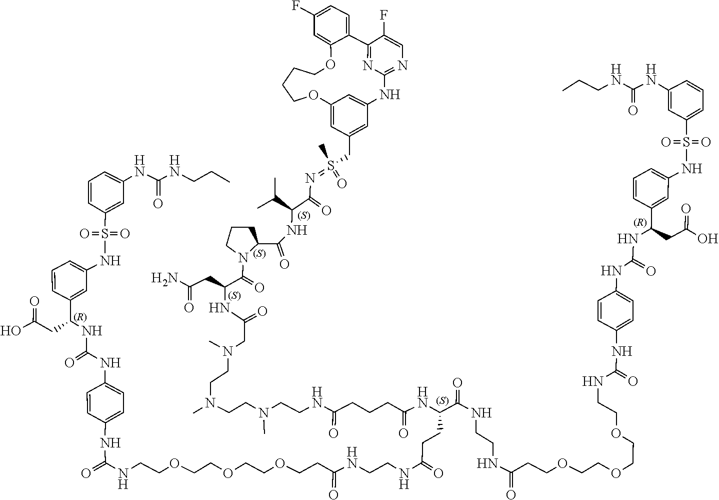

To a solution of trifluoroacetic acid (2S,22S)—N22,N24-bis(15-amino-4-oxo-7,10,13-trioxa-3-azapentadecan-1-yl)-2-[(2S)-2-{[(2S)-1-{[(S)-{[5,23-difluoro-8,13-dioxa-19,21,24-triazatetracyclo[18.3.1.114,18.02,7]pentacosa-1(24),2,4,6,14(25),15,17,20,22-nonaen-16-yl]methyl}(methyl)oxo-lambda6-sulfanylidene]amino}-3-methyl-1-oxobutan-2-yl]carbamoyl}pyrrolidine-1-carbonyl]-6,9,12-trimethyl-4,16,20-trioxo-3,6,9,12,15,21-hexaazatetracosane-1,22,24-tricarboxamide (2/1) (7.00 mg, 95% purity, 3.39 μmol) (Intermediate 63) in DMF (2.0 mL) were added (3R)-3-{[(4-{[(4-nitrophenoxy)carbonyl]amino}phenyl) carbamoyl]amino}-3-[3-({3-[(propylcarbamoyl)amino]benzene-1-sulfonyl}amino)phenyl]propanoic acid (6.83 mg, 9.49 μmol) (Building block 1) and N,N-diisopropylethylamine (3.0 μl, 17 μmol; CAS-RN: [7087-68-5]). The reaction was stirred for 3.5 hours at RT, concentrated in vacuo and the residue was purified by prep HPLC. Another by-product was seen in the HPLC and LC-MS. The product was repurified by HPLC and lyophilized to give the title compound Compound 25 (2.70 mg, 100% purity, 28% yield) as an amorphous residue. LC-MS (Method 3): Rₜ=3.41 min; MS (ESIpos): m/z=2892 [M+H]⁺.

Example C28: Preparation of N-{2-[{2-[(2-{[N2,N6-bis(14-{4-[({(1R)-2-carboxy-1-[3-({3-[(propylcarbamoyl)amino] benzene-1-sulfonyl}amino)phenyl]ethyl}carbamoyl)amino]anilino}-14-oxo-4,7,10-trioxa-13-azatetradecanan-1-oyl)-L-lysyl]amino}ethyl)(methyl)amino]ethyl}(methyl)amino]ethyl}-N-methylglycyl-L-asparaginyl-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (Compound 28)

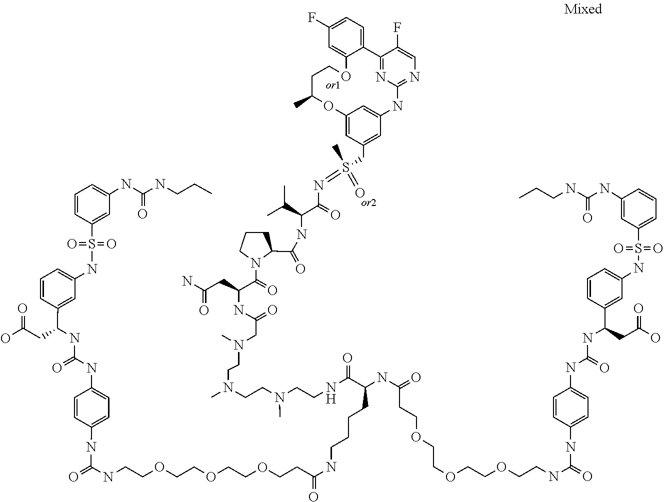

To a solution of trifluoroacetic acid N-{2-[{2-[(2-{[N2,N6-bis(3-{2-[2-(2-aminoethoxy)ethoxy]ethoxy}propanoyl)-L-lysyl]amino}ethyl)(methyl)amino]ethyl}(methyl)amino]ethyl}-N-methylglycyl-L-asparaginyl-L-prolyl-N—[(R*)—{[(4R*)-15,19-difluoro-4-methyl-3,4-dihydro-2H, 11H-12,16-(azeno)-10,6-(metheno)-1,5,11,13-benzodioxadiazacyclooctadecin-8-yl]methyl}(methyl)oxo-lambda6-sulfanylidene]-L-valinamide (2/1) (16.0 mg, 9.08 μmol) (synthesized in analogous fashion to Compound 25) in DMF (6.0 mL) were added Building block 1 (16.3 mg, 22.7 μmol) and N,N-diisopropylethylamine (7.9 μl, 45 μmol; CAS-RN:[7087-68-5]). The reaction was stirred at RT overnight and concentrated in vacuo. The residue was purified by prep. HPLC, then lyophilized to give Compound 28 (12.0 mg, 100% purity, 49% yield) as an amorphous residue. LC-MS (Method 3): Rₜ=3.60 min; MS (ESIpos): m/z=2693 [M+H]⁺. ¹H-NMR (600 MHz, DMSO-d₆) δ[ppm]: 0.833 (2.71), 0.845 (9.02), 0.856 (12.43), 0.866 (5.58), 0.881 (0.79), 1.094 (0.62), 1.104 (0.57), 1.236 (0.77), 1.283 (0.46), 1.363 (0.93), 1.402 (0.54), 1.413 (1.97), 1.423 (4.05), 1.433 (5.74), 1.510 (0.50), 1.615 (0.46), 1.721 (0.59), 1.897 (0.89), 1.989 (0.45), 2.109 (0.56), 2.117 (0.58), 2.127 (0.42), 2.291 (2.92), 2.300 (2.13), 2.332 (0.93), 2.345 (0.87), 2.366 (0.76), 2.407 (0.67), 2.426 (0.94), 2.447 (0.55), 2.603 (0.80), 2.632 (3.84), 2.642 (3.65), 2.800 (2.23), 3.019 (2.20), 3.029 (3.87), 3.038 (3.86), 3.047 (1.81), 3.166 (1.57), 3.197 (6.45), 3.216 (3.94), 3.223 (4.24), 3.421 (16.00), 3.464 (8.57), 3.472 (8.58), 3.487 (8.79), 3.514 (13.77), 3.518 (13.87), 3.570 (2.47), 3.579 (3.87), 3.590 (3.36), 3.682 (0.91), 3.864 (0.45), 4.101 (1.80), 4.109 (1.50), 4.122 (1.01), 4.370 (0.64), 4.460 (0.45), 4.476 (1.31), 4.489 (1.01), 4.717 (2.14), 4.952 (0.53), 4.994 (1.44), 5.005 (1.46), 6.116 (1.20), 6.304 (1.09), 6.488 (1.53), 6.677 (1.38), 6.736 (1.49), 6.896 (1.66), 6.907 (1.91), 6.920 (0.78), 6.932 (1.15), 6.944 (0.60), 6.983 (2.88), 6.994 (2.28), 7.148 (4.30), 7.156 (3.16), 7.168 (1.41), 7.211 (13.86), 7.241 (1.60), 7.252 (2.91), 7.269 (2.05), 7.281 (2.98), 7.292 (1.31), 7.355 (0.90), 7.372 (0.87), 7.400 (2.00), 7.411 (1.59), 7.484 (0.87), 7.627 (0.66), 7.631 (0.65), 7.793 (0.79), 7.805 (0.77), 7.830 (1.04), 8.071 (3.27), 8.089 (0.73), 8.139 (1.09), 8.379 (1.89), 8.399 (1.74), 8.676 (1.50), 8.679 (1.65), 8.708 (1.53), 8.817 (0.89), 8.840 (1.70), 9.748 (1.70), 10.274 (4.74).

Example C29: Preparation of N-{2-[{2-[(2-{[N2,N6-bis(14-{4-[({(1S)-2-carboxy-1-[3-({3-[(propylcarbamoyl) amino]benzene-1-sulfonyl}amino)phenyl]ethyl}carbamoyl)amino]anilino}-14-oxo-4,7,10-trioxa-13-azatetradecanan-1-oyl)-L-lysyl]amino}ethyl)(methyl)amino]ethyl}(methyl) amino]ethyl}-N-methylglycyl-L-asparaginyl-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (Compound 29)

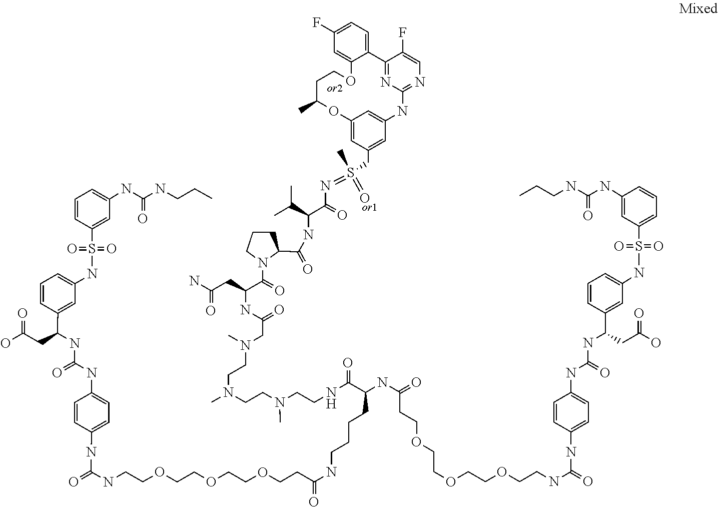

To a solution of trifluoroacetic acid N-{2-[{2-[(2-{[N2,N6-bis(3-{2-[2-(2-aminoethoxy)ethoxy]ethoxy}propanoyl)-L-lysyl]amino}ethyl)(methyl)amino]ethyl}(methyl)amino]ethyl}-N-methylglycyl-L-asparaginyl-L-prolyl-N—[(R*)—{[(4R*)-15,19-difluoro-4-methyl-3,4-dihydro-2H, 11H-12,16-(azeno)-10,6-(metheno)-1,5,11,13-benzodioxadiazacyclooctadecin-8-yl]methyl}(methyl)oxo-lambda6-sulfanylidene]-L-valinamide (2/1) (16.0 mg, 9.08 μmol) (synthesized in analogous fashion to Compound 25) in DMF (6.0 mL) were added Building block 2 (16.3 mg, 22.7 μmol) and N,N-diisopropylethylamine (7.9 μl, 45 μmol; CAS-RN:[7087-68-5]). The reaction was stirred at RT overnight, concentrated in vacuo. The residue was purified by prep. HPLC and lyophilised to give Compound 29 (15.0 mg, 100% purity, 61% yield) as an amorphous residue. LC-MS (Method 3): Rₜ=3.60 min; MS (ESIpos): m/z=2693 [M+H]⁺.

Example C30: Preparation of trisodium 1-[(2S)-15-{[N2,N6-bis(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}-2-(carboxylatomethyl)-4-oxo-7,10,13-trioxa-3-azapentadecanan-1-oyl]-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (Compound 30)

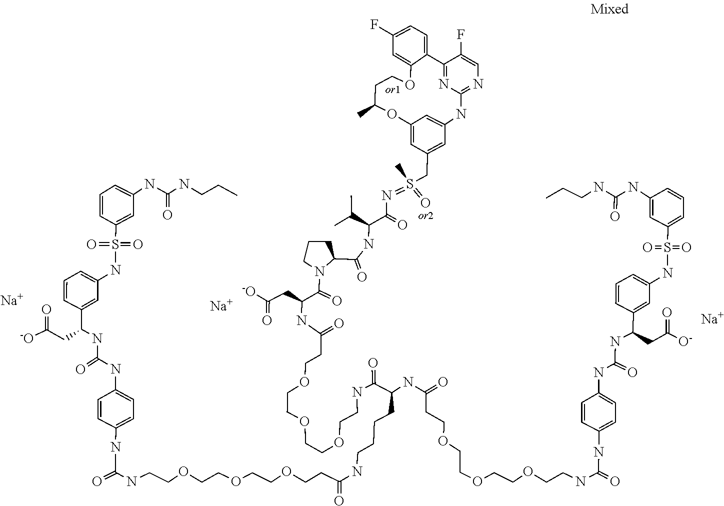

N-(3-{2-[2-(2-{[N2,N6-bis(14-{4-[({(1R)-2-carboxy-1-[3-({3-[(propylcarbamoyl)amino] benzene-1-sulfonyl}amino)phenyl]ethyl}carbamoyl) amino]anilino}-14-oxo-4,7,10-trioxa-13-azatetradecanan-1-oyl)-L-lysyl]amino}ethoxy)ethoxy]ethoxy}propanoyl)-L-alpha-aspartyl-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (55.0 mg, 20.6 μmol) (synthesized in analogous fashion to Compound 25 and using Intermediate 77 and Boc-Lys(Boc)-OSu as a building block) was dissolved in dioxane/water (1:1, 10 mL). A sodium hydroxide solution (62 μl, 1.0 M, 62 μmol) was added. The solution was freeze-dried to give Compound 30 (53.0 mg, 920% purity, 860% yield) as a colorless foam. The payload is building block 18 in this compound. LC-MS (Method 3): Rₜ=4.51 min; MS (ESIpos): 15 m/z=1336 [M+H]e. ¹H-NMR (600 MHz, DMSO-d₆) δ[ppm]: 0.809 (3.08), 0.818 (6.24), 0.830 (10.53), 0.842 (8.09), 0.854 (3.34), 1.234 (1.23), 1.354 (2.72), 1.366 (3.76), 1.378 (3.42), 1.390 (1.82), 1.426 (3.08), 1.436 (3.24), 1.704 (0.68), 1.967 (0.97), 2.105 (0.71), 2.283 (2.31), 2.326 (1.63), 2.368 (1.47), 2.383 (1.73), 2.600 (1.46), 2.938 (2.80), 2.949 (2.79), 3.096 (6.01), 3.210 (3.60), 3.220 (3.66), 3.375 (3.80), 3.416 (3.19), 3.425 (5.00), 3.435 (3.10), 3.476 (10.76), 3.491 (4.73), 3.514 (16.00), 3.567 (7.30), 3.573 (4.44), 3.583 (3.54), 4.080 (1.29), 4.197 (0.67), 4.499 (0.81), 4.709 (1.03), 4.831 (0.78), 4.974 (1.13), 6.151 (0.90), 6.532 (1.63), 6.678 (1.47), 6.744 (1.75), 6.939 (2.06), 6.952 (2.18), 7.062 (1.68), 7.075 (2.72), 7.088 (1.18), 7.192 (4.26), 7.206 (4.93), 7.244 (6.28), 7.258 (3.37), 7.279 (1.75), 7.292 (2.87), 7.306 (1.29), 7.342 (0.88), 7.795 (1.30), 7.922 (1.05), 8.264 (0.75), 8.396 (1.47), 8.709 (2.85), 8.764 (1.22), 9.736 (1.43).

Example C31: Preparation of trisodium 1-[(2S)-15-{[N2,N6-bis(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}-2-(carboxylatomethyl)-4-oxo-7,10,13-trioxa-3-azapentadecanan-1-oyl]-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (Compound 31)

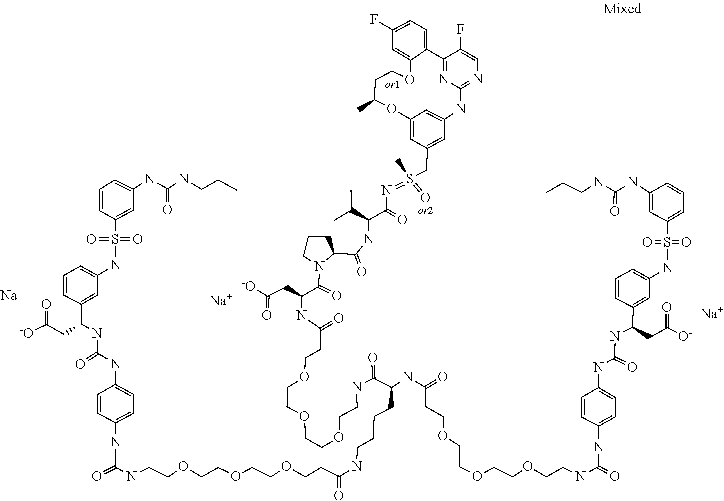

N-(3-{2-[2-(2-{[N2,N6-bis(14-{4-[({(1R)-2-carboxy-1-[3-({3-[(propylcarbamoyl)amino] benzene-1-sulfonyl}amino)phenyl]ethyl}carbamoyl) amino]anilino}-14-oxo-4,7,10-trioxa-13-azatetradecanan-1-oyl)-L-lysyl]amino}ethoxy)ethoxy]ethoxy}propanoyl)-L-alpha-aspartyl-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda6-sulfanylidene]-L-valinamide (50.0 mg, 18.7 μmol) (synthesized in analogous fashion to Compound 25 and using Intermediate 77 and Boc-Lys(Boc)-OSu as a building block) was dissolved in dioxane/water (1:1, 12 mL), then added the NaOH (37 μl, 1.0 M, 37 μmol), dissolved in an ultrasonic bath, then lyophilized to give Compound 31 (53.0 mg, 93% purity, 96% yield) as an amorphous residue. The payload is building block 19 in this compound. LC-MS (Method 3): Rₜ=4.51 min; MS (ESIpos): m/z=2668 [M+H]⁺. ¹H-NMR (600 MHz, DMSO-d₆) δ[ppm]: 0.822 (6.98), 0.840 (11.55), 0.859 (5.29), 1.236 (1.15), 1.373 (2.26), 1.391 (3.30), 1.409 (3.34), 1.425 (4.11), 1.438 (3.18), 1.580 (0.54), 1.842 (0.88), 1.926 (1.15), 2.073 (1.80), 2.283 (2.49), 2.298 (1.80), 2.328 (3.03), 2.367 (2.42), 2.380 (2.30), 2.606 (2.42), 2.670 (1.42), 2.711 (0.88), 2.981 (3.80), 2.996 (3.76), 3.195 (8.29), 3.209 (5.10), 3.223 (5.06), 3.412 (4.83), 3.426 (5.87), 3.457 (7.29), 3.478 (14.54), 3.514 (16.00), 3.573 (3.91), 3.630 (1.30), 4.083 (1.27), 4.187 (0.65), 4.360 (0.58), 4.477 (1.15), 4.707 (2.26), 4.890 (0.61), 4.975 (1.23), 6.080 (1.61), 6.483 (1.46), 6.744 (1.61), 6.808 (1.23), 6.924 (1.30), 6.956 (2.03), 6.976 (2.11), 7.098 (1.46), 7.117 (2.34), 7.137 (1.15), 7.210 (8.90), 7.217 (8.44), 7.266 (1.92), 7.286 (2.65), 7.306 (1.15), 7.344 (0.92), 7.372 (1.07), 7.625 (0.77), 7.690 (1.15), 7.712 (1.23), 7.787 (1.23), 7.837 (0.96), 7.907 (1.11), 7.924 (1.23), 7.946 (0.92), 8.339 (3.03), 8.545 (0.84), 8.681 (1.61), 8.688 (1.57), 8.706 (1.46), 9.748 (1.57), 10.113 (0.73).

Example C32: Preparation of trisodium 1-[(2S)-2-(carboxylatomethyl)-15-{[N6-(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-N2-(14-{4-[({(1S)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}-4-oxo-7,10,13-trioxa-3-azapentadecanan-1-oyl]-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (Compound 32)

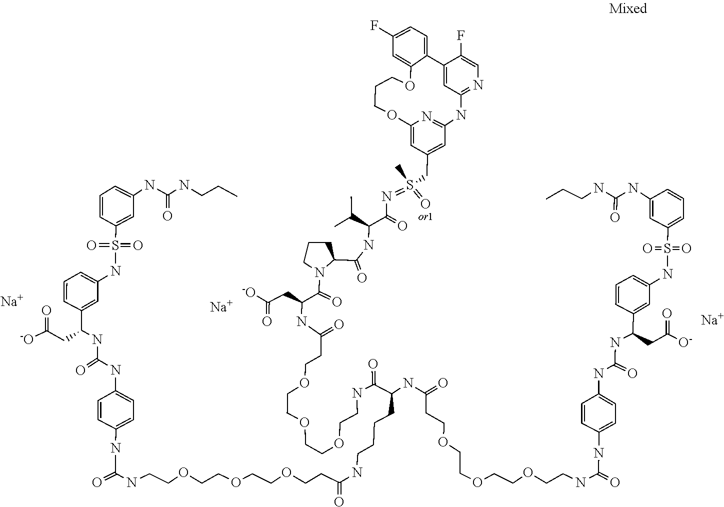

N-(3-{2-[2-(2-{[N6-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-N2-(14-{4-[({(1S)-2-carboxy-1-[3-({3-[(propylcarbamoyl)amino] benzene-1-sulfonyl}amino)phenyl]ethyl}carbamoyl)amino]anilino}-14-oxo-4,7,10-trioxa-13-azatetradecanan-1-oyl)-L-lysyl]amino}ethoxy)ethoxy]ethoxy}propanoyl)-L-alpha-aspartyl-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (41.2 mg, 100% purity, 15.5 μmol) (synthesized in analogous fashion to Compound 25) was dissolved in dioxane/water (1:1, 15 ml). A sodium hydroxide solution (47 μl, 1.0 M, 47 μmol) was added. The solution was freeze-dried to give Compound 32 (41.7 mg, 100% purity, 99% yield) as a colorless foam. Compound 32 contains building block 22 as the payload. LC-MS (Method 3): Rₜ=4.51 min; MS (ESIpos): m/z=2653 [M+H]⁺. ¹H-NMR (600 MHz, DMSO-d₆) δ[ppm]: 0.777 (4.77), 0.793 (5.04), 0.805 (4.48), 0.823 (7.90), 0.842 (4.35), 1.235 (1.09), 1.332 (2.27), 1.350 (3.56), 1.368 (3.19), 1.386 (1.61), 1.465 (0.49), 1.581 (0.49), 1.899 (0.72), 1.987 (0.69), 2.106 (1.22), 2.223 (0.66), 2.281 (2.24), 2.375 (2.01), 2.603 (0.63), 2.670 (0.53), 2.898 (1.25), 2.915 (2.60), 2.930 (2.57), 2.945 (1.35), 2.976 (1.12), 3.192 (3.33), 3.207 (3.16), 3.294 (7.05), 3.402 (3.75), 3.416 (5.10), 3.465 (11.59), 3.507 (16.00), 3.569 (3.46), 3.580 (3.09), 3.805 (0.49), 3.904 (0.49), 4.020 (0.53), 4.037 (0.72), 4.059 (0.56), 4.117 (1.28), 4.190 (0.63), 4.498 (1.32), 4.727 (1.98), 4.776 (0.63), 4.955 (1.09), 6.292 (1.94), 6.472 (0.69), 6.514 (0.69), 6.601 (0.99), 6.665 (1.81), 6.893 (1.28), 6.911 (0.99), 7.050 (1.19), 7.079 (0.86), 7.152 (1.32), 7.170 (1.74), 7.194 (2.04), 7.217 (4.54), 7.237 (5.37), 7.260 (2.63), 7.380 (0.99), 7.501 (2.37), 7.572 (0.69), 7.858 (2.01), 8.003 (1.38), 8.097 (1.48), 8.116 (1.42), 8.175 (0.92), 8.335 (1.38), 8.684 (1.88), 8.701 (1.35), 8.717 (1.22), 8.775 (0.92), 8.848 (1.38), 9.884 (1.55), 11.238 (0.63).

Example C33: Preparation of trisodium 1-[(2S)-2-(carboxylatomethyl)-15-{[N6-(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-N2-(14-{4-[({(1S)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}-4-oxo-7,10,13-trioxa-3-azapentadecanan-1-oyl]-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (Compound 33)

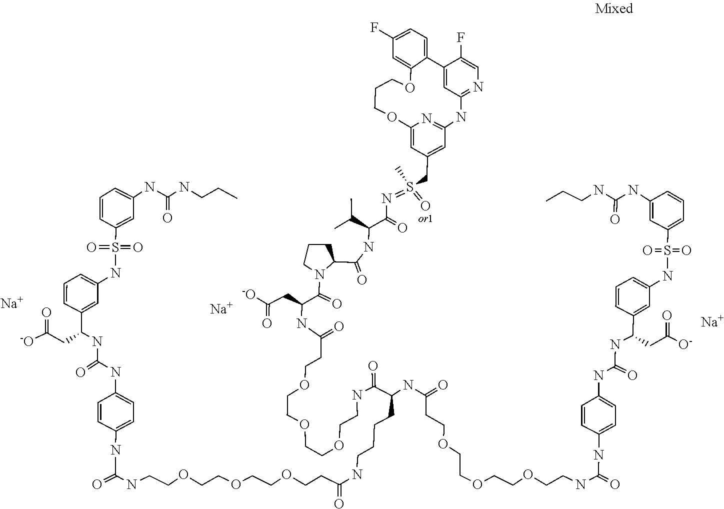

N-(3-{2-[2-(2-{[N6-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-N²-(14-{4-[({(1S)-2-carboxy-1-[3-({3-[(propylcarbamoyl)amino] benzene-1-sulfonyl}amino)phenyl]ethyl}carbamoyl)amino]anilino}-14-oxo-4,7,10-trioxa-13-azatetradecanan-1-oyl)-L-lysyl]amino}ethoxy)ethoxy]ethoxy}propanoyl)-L-alpha-aspartyl-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda⁶-sulfanylidene]-L-valinamide (38.6 mg, 14.5 μmol) (synthesized in analogous fashion to Compound 25, but using Boc-Lys(Boc)-OSu as a building block) was dissolved in dioxane/water (1:1, 10 ml). A sodium hydroxide solution (44 μl, 1.0 M, 44 μmol) was added. The solution was freeze-dried to give Compound 33 (39.2 mg, 100% purity, 99% yield). Compound 33 contains building block 23 as the payload. LC-MS (Method 3): Rₜ=4.51 min; MS (ESIpos): m/z=2656061 [M+H]. ¹H-NMR (600 MHz, DMSO-d₆) δ[ppm]: 0.812 (6.40), 0.822 (9.44), 0.840 (4.52), 1.223 (0.96), 1.332 (2.37), 1.351 (3.63), 1.368 (3.23), 1.387 (1.62), 1.586 (0.52), 1.875 (1.03), 1.963 (0.94), 2.093 (1.34), 2.281 (2.69), 2.372 (2.08), 2.580 (2.27), 2.616 (1.19), 2.670 (0.70), 2.709 (0.47), 2.916 (2.79), 2.932 (2.83), 2.991 (1.41), 3.187 (7.40), 3.210 (4.52), 3.419 (7.64), 3.470 (13.56), 3.508 (16.00), 3.566 (5.76), 3.733 (0.61), 3.833 (0.54), 4.000 (0.75), 4.117 (1.36), 4.191 (0.68), 4.485 (1.29), 4.763 (0.56), 4.797 (1.12), 4.842 (1.15), 4.967 (1.24), 6.306 (1.99), 6.630 (2.93), 6.650 (1.83), 6.889 (0.87), 6.927 (1.57), 6.948 (1.97), 7.039 (1.66), 7.057 (2.46), 7.077 (1.64), 7.166 (1.78), 7.188 (3.89), 7.212 (4.59), 7.239 (4.92), 7.261 (2.39), 7.272 (1.94), 7.294 (2.23), 7.313 (1.27), 7.345 (1.24), 7.508 (2.74), 7.572 (0.73), 7.820 (0.98), 7.853 (2.65), 7.901 (0.66), 7.968 (0.91), 8.043 (0.68), 8.167 (2.11), 8.186 (2.01), 8.339 (1.34), 8.583 (0.84), 8.698 (1.64), 8.843 (2.16), 9.813 (0.98), 11.194 (1.36).

Example C34: Preparation of N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-valyl-N-{4-[({[(S)-[(2-{[5-fluoro-4-(4-fluoro-2-methoxyphenyl)pyridin-2-yl]amino}pyridin-4-yl)methyl](methyl)oxo-lambda6-sulfanylidene]carbamoyl}oxy)methyl]phenyl}-L-alaninamide (Compound 34)

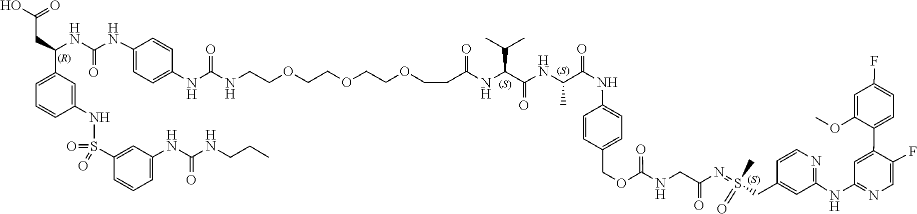

Trifluoroacetic acid N-(3-{2-[2-(2-aminoethoxy)ethoxy]ethoxy}propanoyl)-L-valyl-N-{4-[({[(S)-[(2-{[5-fluoro-4-(4-fluoro-2-methoxyphenyl)pyridin-2-yl]amino}pyridin-4-yl)methyl](methyl)oxo-lambda6-sulfanylidene]carbamoyl}oxy)methyl]phenyl}-L-alaninamide (1/1) (9.20 mg, 8.84 μmol) (Intermediate 79) was dissolved in DMF (3.0 mL). (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (6.36 mg, 8.84 μmol) (Building block 1) and DIEA (15 μl, 88 μmol) were added. The reaction was stirred for 30 minutes at RT and then concentrated in vacuo. The residue was separated by prep. HPLC to give Compound 34 (4.00 mg, 94% purity, 28% yield) as a colorless foam. LC-MS (Method 4): Rₜ=2.81 min; MS (ESIpos): m/z=1507 [M+H]⁺. ¹H-NMR (600 MHz, DMSO-d₆) δ[ppm]: 0.823 (5.91), 0.835 (6.12), 0.842 (5.51), 0.854 (10.83), 0.863 (7.01), 0.867 (7.32), 0.874 (5.77), 1.234 (1.42), 1.292 (5.33), 1.304 (5.11), 1.396 (0.46), 1.408 (1.66), 1.420 (2.86), 1.432 (2.81), 1.445 (1.50), 1.941 (0.62), 1.952 (0.99), 1.963 (0.96), 1.975 (0.60), 2.355 (0.47), 2.366 (0.87), 2.380 (1.03), 2.386 (1.04), 2.390 (1.54), 2.401 (0.66), 2.425 (0.56), 2.444 (0.77), 2.455 (1.42), 2.466 (1.19), 2.516 (1.42), 2.520 (1.49), 2.523 (1.36), 2.614 (0.80), 2.629 (2.71), 2.641 (2.49), 2.654 (0.51), 3.015 (1.14), 3.026 (2.33), 3.036 (2.27), 3.047 (1.06), 3.184 (5.22), 3.212 (2.22), 3.221 (2.30), 3.288 (10.20), 3.415 (3.25), 3.424 (4.94), 3.434 (3.13), 3.465 (3.36), 3.475 (4.54), 3.482 (7.36), 3.486 (8.50), 3.493 (9.08), 3.582 (4.44), 3.586 (4.35), 3.593 (4.97), 3.597 (4.82), 3.800 (16.00), 3.892 (0.55), 3.901 (0.44), 4.195 (1.12), 4.207 (1.36), 4.221 (1.01), 4.372 (0.94), 4.383 (1.36), 4.395 (0.86), 4.425 (0.51), 4.926 (1.80), 4.933 (1.64), 4.960 (5.16), 4.977 (0.66), 4.990 (1.15), 5.003 (1.08), 5.015 (0.43), 6.067 (0.96), 6.208 (0.79), 6.218 (1.43), 6.228 (0.74), 6.622 (1.35), 6.636 (1.25), 6.898 (1.24), 6.911 (2.00), 6.925 (1.56), 6.929 (1.68), 6.939 (1.58), 6.943 (1.55), 6.979 (1.54), 6.992 (1.61), 7.097 (1.43), 7.101 (1.42), 7.116 (1.46), 7.121 (1.45), 7.138 (2.88), 7.141 (3.10), 7.155 (2.54), 7.168 (1.21), 7.188 (0.86), 7.205 (13.12), 7.221 (0.67), 7.240 (1.39), 7.253 (2.51), 7.265 (2.70), 7.270 (3.49), 7.278 (3.45), 7.284 (3.40), 7.291 (1.36), 7.345 (1.14), 7.356 (1.36), 7.370 (1.05), 7.397 (1.41), 7.410 (1.11), 7.570 (3.63), 7.584 (2.90), 7.662 (1.98), 7.872 (1.49), 7.887 (1.41), 8.051 (1.79), 8.054 (2.88), 8.058 (1.70), 8.176 (1.49), 8.187 (1.43), 8.213 (1.93), 8.221 (1.70), 8.248 (2.26), 8.333 (2.78), 8.353 (2.54), 8.765 (3.03), 9.930 (2.50), 10.269 (3.92).

Example C35: Preparation of N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-valyl-N-{4-[({[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]carbamoyl}oxy)methyl]phenyl}-L-alaninamide (Compound 35)

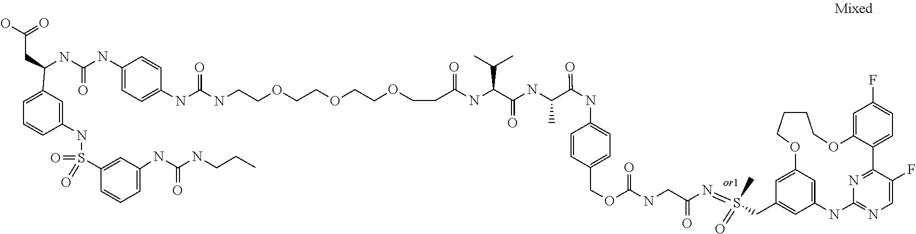

Compound 35 was synthesized using Building block 14 following the same general procedure as described previously to synthesize Compound 34. Compound 35 was obtained as a colorless foam (9.10 mg, 9400 purity, 35% yield).

LC-MS (Method 4): Rₜ=3.45 min; MS (ESIpos): m/z=1563 [M+H]⁺

¹H-NMR (600 MHz, DMSO-d₆) δ[ppm]: 0.827 (6.78), 0.839 (7.37), 0.842 (6.68), 0.855 (11.74), 0.867 (11.74), 0.879 (6.32), 1.299 (6.35), 1.311 (6.07), 1.409 (2.04), 1.421 (3.37), 1.433 (3.34), 1.446 (1.83), 1.859 (3.08), 1.958 (1.36), 1.969 (1.15), 2.393 (1.74), 2.458 (1.65), 2.630 (3.51), 2.642 (2.97), 3.036 (2.21), 3.169 (13.52), 3.217 (2.21), 3.416 (2.46), 3.426 (4.44), 3.435 (2.12), 3.488 (6.28), 3.494 (6.36), 3.595 (2.76), 3.853 (2.53), 4.132 (1.83), 4.201 (1.28), 4.215 (1.64), 4.226 (1.25), 4.268 (2.74), 4.391 (1.68), 4.403 (1.26), 4.728 (5.54), 4.933 (1.04), 4.953 (2.59), 4.967 (2.84), 4.989 (1.78), 5.004 (1.46), 6.061 (0.85), 6.201 (1.21), 6.507 (2.71), 6.614 (1.44), 6.629 (1.46), 6.714 (3.41), 6.876 (1.78), 6.896 (1.57), 6.910 (1.62), 6.981 (1.65), 6.994 (1.82), 7.142 (4.72), 7.156 (3.84), 7.169 (1.56), 7.206 (16.00), 7.241 (1.36), 7.255 (2.92), 7.266 (2.80), 7.272 (4.14), 7.279 (3.61), 7.286 (4.27), 7.396 (2.58), 7.408 (1.95), 7.558 (4.62), 7.573 (3.64), 7.876 (1.89), 7.890 (1.68), 8.009 (2.62), 8.053 (3.41), 8.175 (1.71), 8.186 (1.89), 8.327 (3.01), 8.347 (3.01), 8.662 (3.98), 8.665 (3.78), 8.748 (3.61), 9.849 (4.09), 9.918 (3.07), 10.268 (4.73).

Example C36: Preparation of disodium 1-[(2S)-2-(carboxylatomethyl)-17-{4-[[({(1R)-2-carboxylato-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda⁶-sulfanylidene]-L-valinamide (Compound 36)

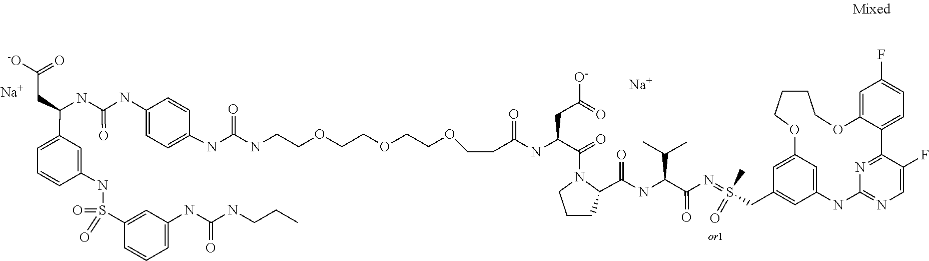

N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxo-lambda6-sulfanylidene]-L-valinamide (21.2 mg, 13.6 μmol) (Intermediate 84) was dissolved in dioxane/water (1:1; 5.0 ml). A sodium hydroxide solution (27 μl, 1.0 M, 27 μmol) was added. The solution was freeze-dried to give Compound 36 (21.5 mg, 100% purity, 9900 yield) as a colorless foam. LC-MS (Method 3): Rₜ=4.32 min; MS (ESIpos): m/z=1553 [M+H]⁺. ¹H-NMR (600 MHz, DMSO-d6) [ppm]: 0.814 (8.11), 0.827 (16.00), 0.839 (10.20), 1.235 (0.47), 1.343 (1.54), 1.355 (3.23), 1.367 (3.01), 1.379 (1.42), 1.823 (0.98), 1.874 (2.13), 1.886 (1.93), 1.935 (0.48), 1.949 (0.98), 1.961 (1.23), 2.111 (0.51), 2.121 (0.84), 2.133 (0.83), 2.144 (0.46), 2.225 (0.76), 2.385 (1.20), 2.396 (0.63), 2.408 (0.47), 2.424 (0.53), 2.519 (2.46), 2.581 (1.39), 2.610 (1.21), 2.909 (0.84), 2.919 (2.15), 2.931 (2.16), 2.941 (0.74), 3.093 (10.79), 3.163 (0.60), 3.173 (0.59), 3.212 (0.68), 3.221 (0.78), 3.244 (0.45), 3.418 (1.45), 3.426 (2.36), 3.433 (2.22), 3.441 (1.82), 3.450 (2.08), 3.457 (1.74), 3.464 (1.63), 3.470 (1.76), 3.491 (3.27), 3.499 (3.71), 3.515 (9.14), 3.531 (1.97), 3.540 (0.68), 3.568 (0.43), 3.604 (0.47), 3.618 (0.68), 3.627 (0.70), 3.715 (0.52), 3.727 (0.53), 3.800 (0.54), 4.013 (1.04), 4.023 (1.09), 4.027 (1.15), 4.037 (0.99), 4.131 (0.91), 4.264 (1.90), 4.474 (0.89), 4.480 (1.04), 4.487 (0.97), 4.492 (0.89), 4.716 (1.06), 4.739 (1.45), 4.800 (0.70), 4.811 (0.70), 4.838 (1.41), 4.861 (1.06), 4.964 (0.82), 6.553 (2.52), 6.636 (1.19), 6.650 (1.26), 6.675 (2.51), 6.844 (0.66), 6.848 (0.68), 6.858 (1.30), 6.862 (1.41), 6.872 (0.81), 6.875 (0.83), 6.932 (1.27), 6.944 (1.56), 7.043 (1.41), 7.056 (2.30), 7.069 (1.06), 7.130 (1.25), 7.145 (1.25), 7.168 (1.57), 7.181 (1.82), 7.209 (2.16), 7.223 (3.80), 7.249 (3.95), 7.264 (1.93), 7.278 (1.73), 7.292 (2.44), 7.305 (1.42), 7.332 (0.91), 7.347 (0.93), 7.361 (0.86), 7.366 (0.92), 7.378 (1.20), 7.387 (0.88), 7.392 (0.80), 7.501 (2.18), 7.866 (2.01), 7.925 (0.72), 8.016 (2.40), 8.125 (0.72), 8.186 (1.18), 8.200 (1.19), 8.738 (2.36), 8.793 (0.75), 8.822 (1.79), 9.822 (1.42), 9.875 (0.70), 11.270 (1.00).

Example C37: Preparation of N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda⁶-sulfanylidene]-L-valinamide (Compound 37)

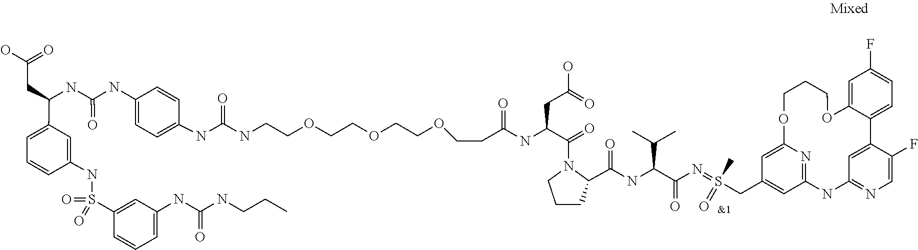

Compound 37 was synthesized using Building block B22-4 following the same general procedure as described previously to synthesize Compound 36. Compound 37 was obtained as a yellow foam (8.60 mg, 96% purity, 72% yield). LC-MS (Method 3): Rₜ=4.46 min; MS (ESIpos): m/z=1542 [M+H]⁺. ¹H-NMR (600 MHz, DMSO-d6) δ[ppm]: 0.070 (1.08), 0.814 (3.70), 0.822 (4.75), 0.825 (4.84), 0.833 (7.08), 0.843 (8.18), 0.855 (15.24), 0.867 (9.79), 1.397 (0.52), 1.410 (2.13), 1.422 (3.81), 1.433 (3.75), 1.446 (2.00), 1.458 (0.43), 1.832 (0.61), 1.841 (0.81), 1.850 (0.91), 1.901 (0.52), 1.915 (0.96), 1.925 (1.29), 1.936 (1.04), 1.946 (0.89), 1.957 (1.21), 1.966 (0.83), 2.083 (2.09), 2.095 (2.07), 2.332 (2.47), 2.342 (1.27), 2.386 (0.49), 2.397 (0.63), 2.402 (0.64), 2.410 (0.66), 2.416 (0.69), 2.425 (1.13), 2.430 (0.83), 2.438 (0.72), 2.444 (0.67), 2.520 (0.97), 2.614 (0.69), 2.631 (3.48), 2.642 (3.32), 2.654 (0.66), 2.681 (0.66), 2.691 (0.73), 2.697 (0.71), 2.706 (0.88), 2.718 (0.58), 2.724 (0.67), 2.733 (0.54), 3.016 (1.15), 3.027 (2.39), 3.037 (2.35), 3.048 (1.08), 3.156 (0.60), 3.182 (7.01), 3.217 (2.45), 3.266 (0.72), 3.279 (7.05), 3.417 (2.77), 3.426 (4.83), 3.436 (2.51), 3.459 (2.84), 3.467 (4.06), 3.480 (4.53), 3.484 (3.61), 3.488 (3.05), 3.504 (1.39), 3.560 (2.05), 3.571 (3.63), 3.581 (1.92), 3.625 (1.70), 3.637 (1.32), 3.675 (0.78), 3.948 (2.27), 4.064 (1.87), 4.072 (2.26), 4.080 (2.12), 4.087 (2.12), 4.095 (1.68), 4.114 (2.67), 4.121 (3.22), 4.475 (0.93), 4.485 (1.52), 4.495 (1.40), 4.504 (1.55), 4.514 (2.09), 4.523 (1.53), 4.723 (0.65), 4.746 (1.85), 4.770 (1.47), 4.788 (0.56), 4.853 (0.41), 4.865 (1.45), 4.876 (0.95), 4.887 (1.52), 4.897 (0.74), 4.979 (0.62), 4.991 (1.41), 5.004 (1.39), 5.016 (0.59), 6.062 (0.71), 6.203 (1.21), 6.266 (2.39), 6.317 (2.56), 6.580 (2.22), 6.603 (2.25), 6.615 (1.68), 6.629 (1.51), 6.882 (0.78), 6.886 (0.90), 6.900 (3.16), 6.914 (2.52), 6.981 (1.77), 6.994 (1.99), 7.063 (1.45), 7.067 (1.47), 7.082 (1.59), 7.086 (1.42), 7.139 (3.30), 7.143 (3.44), 7.156 (3.29), 7.169 (1.54), 7.189 (0.71), 7.205 (16.00), 7.221 (0.71), 7.241 (1.41), 7.255 (3.01), 7.267 (2.60), 7.280 (3.43), 7.293 (1.43), 7.397 (1.80), 7.411 (1.53), 7.567 (0.58), 7.579 (1.05), 7.598 (0.61), 7.687 (0.91), 7.702 (0.88), 7.761 (0.97), 7.776 (0.88), 8.054 (3.76), 8.319 (2.14), 8.326 (6.22), 8.347 (3.09), 8.686 (1.58), 8.690 (1.77), 8.696 (1.59), 8.700 (1.57), 8.751 (4.00), 9.631 (2.01), 9.685 (1.99), 10.267 (5.07).

Example C38: Preparation of N-{14-[4-({[(1S)-2-carboxy-1-{3-[({3-[(propylcarbamoyl)amino]phenyl}sulfonyl)amino]phenyl}ethyl]carbamoyl}amino)anilino]-14-oxo-4,7,10-trioxa-13-azatetradecan-1-oyl}-L-alpha-aspartyl-L-prolyl-N—[(RS)—{[15,19-difluoro-3,4-dihydro-2H,11H-10,6-(azeno)-12,16-(metheno)-1,5,11,13-benzodioxadiazacyclooctadecin-8-yl]methyl}(methyl)oxido-lambda⁶-sulfanylidene]-L-valinamide (Compound 38)

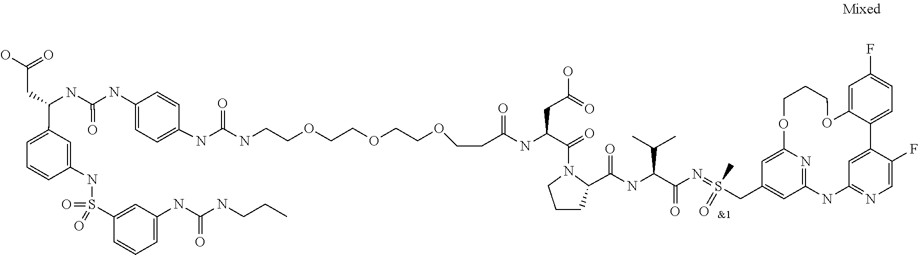

Compound 38 was synthesized using Building block B22-4 following the same general procedure as described previously to synthesize Compound 36. Compound 38 was obtained as a yellow foam (8.12 mg, 100% purity, 71% yield). LC-MS (Method 3): Rₜ=4.46 min; MS (ESIpos): m/z=1541 [M+H]⁺. ¹H-NMR (600 MHz, DMSO-d₆) δ[ppm]: 0.814 (3.63), 0.823 (4.60), 0.825 (4.83), 0.834 (7.00), 0.843 (7.90), 0.855 (14.59), 0.868 (9.50), 1.236 (0.47), 1.398 (0.47), 1.410 (2.04), 1.422 (3.67), 1.434 (3.67), 1.446 (1.92), 1.458 (0.44), 1.841 (0.76), 1.850 (0.91), 1.915 (0.96), 1.926 (1.29), 1.935 (1.03), 1.947 (0.87), 1.957 (1.19), 1.966 (0.82), 2.084 (2.08), 2.094 (2.06), 2.332 (2.39), 2.386 (0.50), 2.397 (0.57), 2.403 (0.61), 2.410 (0.65), 2.416 (0.60), 2.425 (1.08), 2.430 (0.81), 2.437 (0.67), 2.444 (0.61), 2.520 (1.07), 2.615 (0.68), 2.631 (3.48), 2.642 (3.33), 2.654 (0.63), 2.681 (0.65), 2.691 (0.78), 2.696 (0.70), 2.706 (0.85), 2.718 (0.57), 2.724 (0.68), 2.733 (0.54), 3.016 (1.12), 3.027 (2.37), 3.037 (2.37), 3.048 (1.08), 3.157 (0.60), 3.182 (6.80), 3.217 (2.41), 3.267 (0.74), 3.279 (6.99), 3.418 (2.72), 3.427 (4.78), 3.436 (2.52), 3.459 (2.79), 3.467 (4.04), 3.480 (4.48), 3.484 (3.61), 3.488 (3.06), 3.504 (1.42), 3.561 (2.02), 3.571 (3.66), 3.582 (1.91), 3.625 (1.75), 3.638 (1.43), 3.675 (0.90), 3.896 (2.59), 4.064 (1.48), 4.072 (1.89), 4.080 (1.83), 4.087 (1.81), 4.095 (1.39), 4.114 (2.44), 4.122 (3.03), 4.476 (0.87), 4.485 (1.53), 4.495 (1.34), 4.504 (1.49), 4.514 (2.09), 4.523 (1.54), 4.723 (0.70), 4.747 (1.82), 4.770 (1.46), 4.788 (0.60), 4.865 (1.46), 4.876 (0.96), 4.888 (1.50), 4.897 (0.70), 4.980 (0.57), 4.992 (1.40), 5.005 (1.41), 5.016 (0.56), 6.062 (0.70), 6.203 (1.22), 6.266 (2.39), 6.317 (2.53), 6.580 (2.16), 6.603 (2.25), 6.615 (1.70), 6.629 (1.48), 6.882 (0.74), 6.886 (0.91), 6.900 (3.14), 6.914 (2.50), 6.981 (1.70), 6.994 (1.99), 7.064 (1.43), 7.068 (1.45), 7.083 (1.56), 7.087 (1.39), 7.139 (3.27), 7.143 (3.42), 7.156 (3.18), 7.169 (1.49), 7.190 (0.67), 7.206 (16.00), 7.222 (0.73), 7.242 (1.35), 7.255 (2.99), 7.267 (2.55), 7.280 (3.35), 7.293 (1.36), 7.398 (1.74), 7.411 (1.46), 7.567 (0.56), 7.586 (1.00), 7.598 (0.61), 7.688 (0.90), 7.703 (0.87), 7.761 (0.91), 7.776 (0.89), 8.054 (3.70), 8.057 (2.27), 8.319 (2.12), 8.326 (6.16), 8.347 (3.11), 8.686 (1.61), 8.690 (1.74), 8.696 (1.61), 8.700 (1.50), 8.751 (3.89), 9.631 (1.95), 9.685 (1.93), 10.268 (4.94).

Example C39: Preparation of N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxo-lambda⁶-sulfanylidene]-D-valinamide (Compound 39)

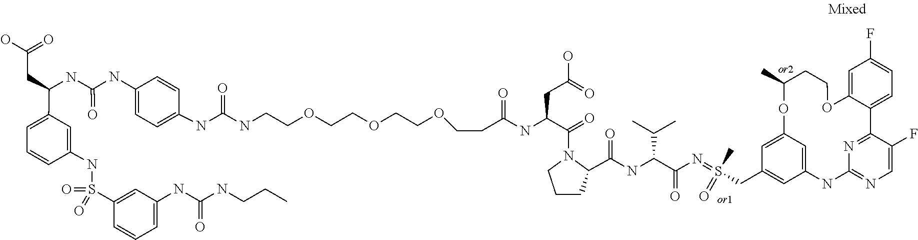

Compound 39 was synthesized using Example B7 following the same general procedure as described previously to synthesize Compound 36. Compound 39 was obtained as an amorphous residue (8.00 mg, 100% purity, 56% yield). LC-MS (Method 3): Rₜ=4.46 min; MS (ESIpos): m/z=1556 [M+H]⁺. ¹H-NMR (600 MHz, DMSO-d₆) δ[ppm]: −0.100 (0.86), 0.097 (0.91), 0.824 (6.72), 0.836 (11.47), 0.842 (7.45), 0.849 (7.86), 0.854 (12.51), 0.867 (6.15), 1.397 (0.59), 1.409 (2.23), 1.421 (4.67), 1.427 (6.45), 1.436 (6.66), 1.445 (2.44), 1.717 (1.02), 1.827 (1.02), 1.839 (1.19), 1.916 (1.28), 1.927 (2.00), 2.075 (0.80), 2.085 (1.09), 2.096 (1.09), 2.106 (0.71), 2.316 (2.21), 2.327 (3.97), 2.338 (2.57), 2.396 (1.16), 2.409 (1.19), 2.424 (2.03), 2.437 (1.27), 2.519 (2.39), 2.629 (3.51), 2.641 (3.35), 2.653 (0.96), 2.684 (1.18), 2.694 (1.30), 2.712 (1.14), 2.721 (0.98), 3.015 (1.37), 3.026 (2.87), 3.036 (2.83), 3.047 (1.34), 3.161 (1.03), 3.196 (13.26), 3.212 (2.67), 3.220 (2.78), 3.417 (3.37), 3.426 (5.20), 3.436 (2.87), 3.459 (3.71), 3.467 (5.08), 3.480 (5.59), 3.488 (4.31), 3.558 (6.68), 3.569 (10.05), 3.580 (8.77), 3.609 (8.64), 3.620 (8.57), 4.070 (1.60), 4.079 (2.05), 4.084 (2.53), 4.094 (1.96), 4.362 (0.94), 4.453 (0.80), 4.474 (2.21), 4.486 (1.51), 4.681 (0.62), 4.706 (4.65), 4.867 (0.68), 4.880 (1.30), 4.890 (1.23), 4.903 (0.52), 4.977 (0.61), 4.990 (1.46), 5.003 (1.37), 5.015 (0.61), 6.057 (1.02), 6.197 (1.62), 6.484 (2.85), 6.611 (1.62), 6.624 (1.57), 6.742 (2.99), 6.898 (1.60), 6.912 (2.57), 6.926 (1.64), 6.936 (0.89), 6.980 (1.76), 6.993 (2.03), 7.137 (3.28), 7.141 (3.15), 7.155 (3.10), 7.168 (1.39), 7.204 (16.00), 7.240 (1.39), 7.253 (2.85), 7.266 (2.42), 7.279 (3.15), 7.292 (1.30), 7.348 (1.51), 7.369 (1.43), 7.396 (1.84), 7.410 (1.51), 7.613 (0.71), 7.626 (1.10), 7.633 (1.07), 7.647 (0.66), 7.677 (1.71), 7.692 (1.66), 8.052 (3.60), 8.303 (1.94), 8.316 (2.42), 8.323 (3.40), 8.343 (3.17), 8.681 (3.30), 8.686 (3.14), 8.705 (2.87), 8.744 (3.96), 9.744 (3.63), 10.266 (4.92).

Example C40: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[16,20-difluoro-2,3,4,5-tetrahydro-12H-13,17-(azeno)-11,7-(metheno)-1,6,12,14-benzodioxadiazacyclononadecin-9-yl]methyl}(methyl)oxido-lambda⁶-sulfanylidene]-L-valinamide (Compound 40)

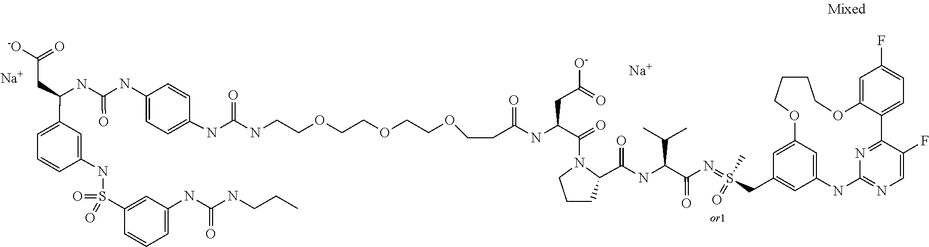

Compound 40 was synthesized using Building block 15 following the same general procedure as described previously to synthesize Compound 36. Compound 40 was obtained as a colorless foam (10.00 mg, 98% purity, 98% yield). LC-MS (Method 3): Rₜ=4.31 min; MS (ESIpos): m/z=1554 [M+H]⁺. ¹H-NMR (600 MHz, DMSO-d₆) δ[ppm]: 0.820 (5.43), 0.824 (5.60), 0.831 (9.69), 0.837 (9.76), 0.842 (5.44), 0.849 (4.91), 1.223 (0.43), 1.234 (1.01), 1.356 (0.53), 1.368 (1.19), 1.380 (2.12), 1.392 (2.03), 1.404 (1.09), 1.861 (2.38), 1.898 (1.14), 1.909 (1.02), 1.924 (0.63), 2.064 (0.52), 2.075 (0.76), 2.085 (0.73), 2.096 (0.53), 2.310 (0.77), 2.321 (1.63), 2.330 (1.63), 2.341 (0.66), 2.386 (1.14), 2.399 (0.60), 2.413 (0.73), 2.425 (0.83), 2.517 (1.65), 2.520 (1.67), 2.523 (1.60), 2.567 (0.86), 2.578 (0.72), 2.600 (0.90), 2.609 (1.15), 2.627 (0.53), 2.635 (0.43), 2.654 (0.45), 2.694 (0.62), 2.703 (0.72), 2.721 (0.63), 2.731 (0.84), 2.890 (0.41), 2.950 (0.77), 2.960 (1.57), 2.971 (1.53), 3.162 (0.90), 3.176 (9.44), 3.210 (2.02), 3.219 (2.08), 3.228 (0.90), 3.419 (2.12), 3.428 (3.51), 3.437 (1.78), 3.458 (2.24), 3.466 (3.10), 3.482 (3.28), 3.486 (2.26), 3.490 (2.40), 3.506 (1.61), 3.517 (16.00), 3.558 (1.67), 3.568 (3.18), 3.579 (1.54), 3.616 (1.04), 3.627 (1.68), 3.638 (0.98), 4.041 (1.02), 4.050 (1.05), 4.055 (1.02), 4.064 (0.95), 4.131 (1.28), 4.269 (1.82), 4.463 (0.87), 4.469 (0.84), 4.476 (0.88), 4.482 (0.79), 4.717 (3.30), 4.865 (0.41), 4.879 (0.80), 4.888 (0.79), 4.972 (0.77), 4.985 (0.74), 6.513 (2.15), 6.655 (2.17), 6.749 (0.48), 6.857 (0.62), 6.861 (0.60), 6.871 (1.14), 6.875 (1.18), 6.885 (0.60), 6.889 (0.62), 6.949 (1.15), 6.961 (1.30), 7.083 (0.93), 7.096 (1.47), 7.109 (0.73), 7.138 (1.09), 7.142 (1.09), 7.158 (1.05), 7.195 (2.86), 7.210 (4.98), 7.230 (3.80), 7.245 (1.54), 7.272 (1.39), 7.285 (2.06), 7.299 (1.01), 7.378 (0.62), 7.383 (0.67), 7.392 (0.95), 7.404 (0.62), 7.409 (0.55), 7.729 (1.25), 7.744 (1.11), 7.984 (1.81), 8.290 (1.05), 8.304 (0.95), 8.401 (0.42), 8.655 (2.89), 8.658 (2.68), 9.704 (2.22).

Example C41: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxido-lambda⁶-sulfanylidene]-L-valinamide (Compound 41)

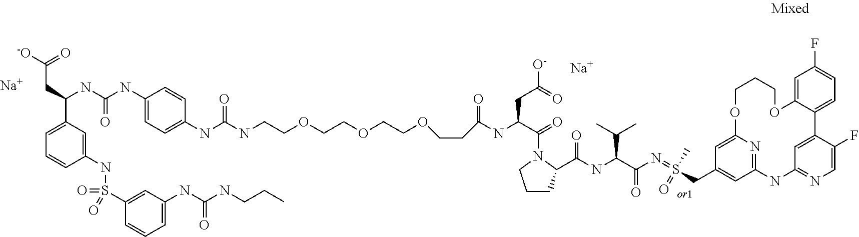

Compound 41 was synthesized using Building block 23 following the same general procedure as described previously to synthesize Compound 36. Compound 41 was obtained as an amorphous residue (47.70 mg, 100% purity, 97% yield). LC-MS (Method 3): Rₜ=4.42 min; MS (ESIpos): m/z=1540 [M+H]⁺.

Example C42: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxido-lambda⁶-sulfanylidene]-L-valinamide (Compound 42)

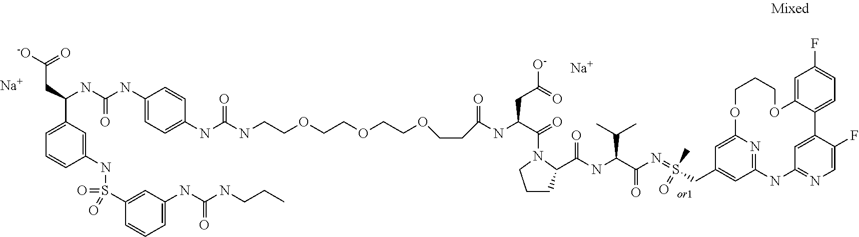

Compound 42 was synthesized using Building block 22 following the same general procedure as described previously to synthesize Compound 36. Compound 42 was obtained as an amorphous residue (28.00 mg, 100% purity, 97% yield). LC-MS (Method 3): Rₜ=4.45 min; MS (ESIpos): m/z=1540 [M+H]⁺.

Example C43: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[18,22-difluoro-2,3,4,5,6,7-hexahydro-14H-13,9-(azeno)-15,19-(metheno)-1,8,14,16-benzodioxadiazacyclohenicosin-11-yl]methyl}(methyl)oxido-lambda⁶-sulfanylidene]-L-valinamide (Compound 43)

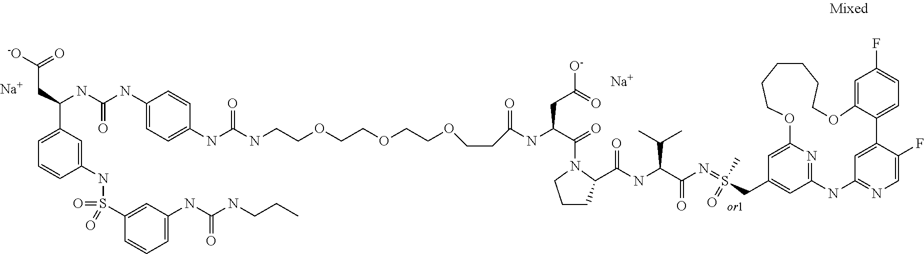

Compound 43 was synthesized using Building block 20 following the same general procedure as described previously to synthesize Compound 36. Compound 43 was obtained as an amorphous residue (16.00 mg, 99% purity, 96% yield). LC-MS (Method 3): Rₜ=4.68 min; MS (ESIpos): m/z=1582 [M+H]⁺.

Example C44: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[18,22-difluoro-2,3,4,5,6,7-hexahydro-14H-13,9-(azeno)-15,19-(metheno)-1,8,14,16-benzodioxadiazacyclohenicosin-11-yl]methyl}(methyl)oxido-lambda⁶-sulfanylidene]-L-valinamide (Compound 44)

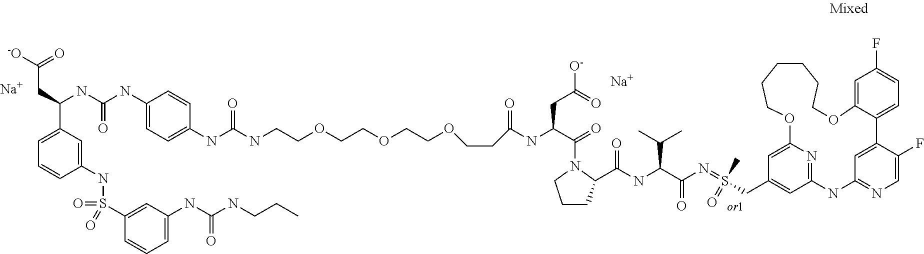

Compound 44 was synthesized using Building block 21 following the same general procedure as described previously to synthesize Compound 36. Compound 44 was obtained as an amorphous residue (24.0 mg, 99% purity, 96% yield). LC-MS (Method 3): Rₜ=4.68 min; MS (ESIpos): m/z=1582 [M+H]⁺.

Example C45: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxido-lambda⁶-sulfanylidene]-L-valinamide (Compound 45)

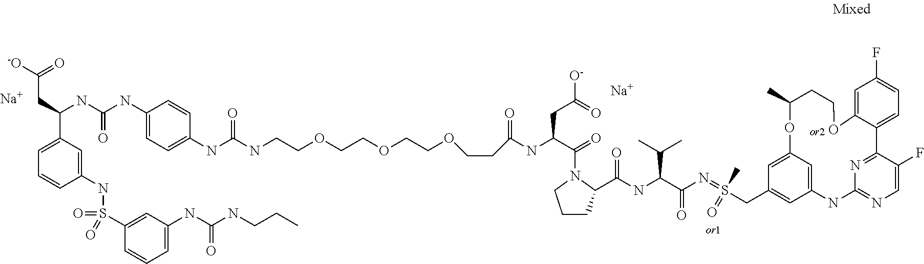

Compound 45 was synthesized using Building block 16 following the same general procedure as described previously to synthesize Compound 36. Compound 45 was obtained as an amorphous residue (21.0 mg, 98% purity, 96% yield). LC-MS (Method 3): Rₜ=4.45 min; MS (ESIpos): m/z=1554 [M+H]⁺. ¹H-NMR (600 MHz, DMSO-d₆) δ[ppm]: 0.830 (4.48), 0.837 (5.75), 0.842 (9.18), 0.848 (5.52), 0.855 (4.45), 0.869 (5.02), 0.881 (4.86), 0.901 (0.46), 1.370 (0.41), 1.382 (1.19), 1.394 (2.12), 1.406 (2.14), 1.418 (1.61), 1.426 (4.49), 1.436 (4.29), 1.730 (0.71), 1.846 (0.65), 1.923 (0.55), 1.965 (1.06), 1.977 (1.06), 2.105 (0.55), 2.116 (0.75), 2.126 (0.72), 2.137 (0.53), 2.322 (1.16), 2.331 (2.27), 2.341 (1.38), 2.357 (0.85), 2.386 (0.88), 2.411 (0.69), 2.425 (1.16), 2.439 (0.93), 2.453 (0.94), 2.516 (1.94), 2.520 (1.83), 2.523 (1.68), 2.590 (0.84), 2.610 (1.43), 2.617 (1.38), 2.635 (0.46), 2.653 (0.49), 2.710 (0.75), 2.719 (0.87), 2.737 (0.77), 2.746 (0.62), 2.983 (1.58), 2.994 (1.56), 3.051 (0.74), 3.084 (8.90), 3.200 (1.00), 3.210 (2.08), 3.219 (2.17), 3.228 (0.93), 3.397 (0.66), 3.417 (2.06), 3.426 (3.52), 3.436 (1.90), 3.458 (2.28), 3.462 (2.24), 3.466 (3.18), 3.480 (3.95), 3.485 (2.34), 3.488 (2.33), 3.516 (16.00), 3.560 (1.83), 3.568 (6.54), 3.581 (1.69), 3.610 (0.68), 3.623 (0.85), 3.659 (0.71), 4.051 (1.03), 4.060 (1.15), 4.065 (1.16), 4.074 (1.15), 4.085 (0.83), 4.102 (0.77), 4.352 (0.66), 4.461 (0.55), 4.481 (0.91), 4.500 (0.46), 4.524 (0.87), 4.530 (0.90), 4.536 (0.91), 4.670 (1.16), 4.692 (1.37), 4.871 (1.18), 4.894 (1.50), 4.908 (0.91), 4.921 (0.47), 4.978 (0.91), 4.991 (0.93), 5.002 (0.44), 6.105 (0.63), 6.559 (2.12), 6.691 (2.21), 6.805 (0.52), 6.909 (0.74), 6.923 (1.21), 6.933 (0.74), 6.937 (0.75), 6.959 (1.25), 6.972 (1.38), 7.103 (0.78), 7.117 (1.28), 7.129 (0.63), 7.195 (1.28), 7.210 (5.23), 7.218 (4.80), 7.233 (1.33), 7.269 (1.50), 7.282 (2.14), 7.295 (1.06), 7.352 (1.12), 7.369 (1.12), 7.600 (0.59), 7.614 (0.84), 7.621 (0.83), 7.633 (0.65), 7.808 (1.28), 7.822 (1.28), 8.315 (1.27), 8.328 (1.33), 8.365 (0.99), 8.674 (2.45), 8.679 (2.42), 8.713 (1.97), 9.746 (2.46).

Example C46: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxido-lambda⁶-sulfanylidene]-L-valinamide (Compound 46)

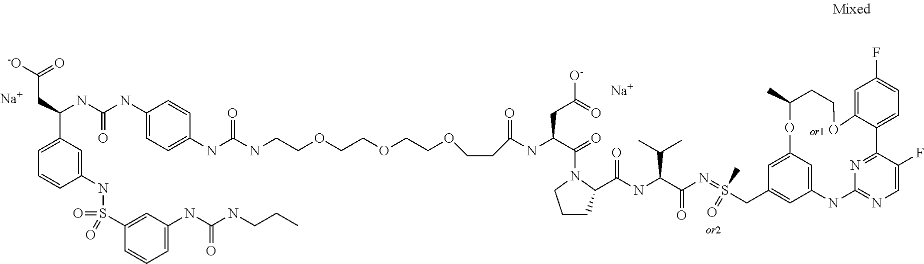

Compound 46 was synthesized using Building block 17 following the same general procedure as described previously to synthesize Compound 36. Compound 46 was obtained as an amorphous residue (20.00 mg, 98% purity, 98% yield). LC-MS (Method 3): Rₜ=4.44 min; MS (ESIpos): m/z=1554 [M+H]⁺. ¹H-NMR (600 MHz, DMSO-d₆) δ[ppm]: 0.817 (5.15), 0.821 (5.41), 0.833 (11.13), 0.842 (5.34), 0.845 (5.55), 0.860 (0.90), 0.871 (0.76), 1.359 (1.25), 1.371 (2.32), 1.383 (2.29), 1.395 (1.19), 1.426 (4.35), 1.436 (4.29), 1.718 (0.73), 1.736 (0.41), 1.830 (0.59), 1.838 (0.70), 1.868 (0.58), 1.881 (0.58), 1.909 (0.60), 1.922 (0.95), 1.935 (1.10), 2.071 (0.50), 2.082 (0.72), 2.093 (0.72), 2.103 (0.47), 2.303 (0.66), 2.314 (1.15), 2.323 (1.28), 2.334 (1.31), 2.344 (1.24), 2.358 (0.64), 2.375 (0.59), 2.389 (0.96), 2.403 (0.61), 2.416 (0.59), 2.425 (0.48), 2.517 (1.77), 2.520 (1.71), 2.523 (1.57), 2.562 (0.84), 2.594 (0.93), 2.602 (1.02), 2.614 (0.78), 2.620 (0.74), 2.629 (0.55), 2.659 (0.68), 2.669 (0.72), 2.687 (0.62), 2.696 (0.55), 2.935 (0.79), 2.946 (1.72), 2.956 (1.70), 2.967 (0.72), 3.209 (2.68), 3.220 (10.58), 3.418 (2.09), 3.427 (3.49), 3.436 (1.75), 3.455 (2.31), 3.463 (3.29), 3.480 (3.35), 3.488 (2.35), 3.499 (1.57), 3.515 (16.00), 3.556 (1.24), 3.568 (7.69), 3.579 (1.12), 3.636 (1.43), 4.056 (1.02), 4.065 (1.05), 4.071 (1.10), 4.080 (1.38), 4.103 (0.75), 4.351 (0.52), 4.368 (0.63), 4.453 (0.53), 4.471 (0.92), 4.484 (1.05), 4.491 (1.30), 4.657 (0.86), 4.679 (1.42), 4.716 (1.47), 4.739 (0.89), 4.843 (0.40), 4.856 (0.83), 4.865 (0.82), 4.969 (0.74), 6.474 (2.04), 6.700 (0.63), 6.734 (2.15), 6.911 (0.66), 6.924 (1.12), 6.939 (1.41), 6.954 (1.37), 7.070 (1.00), 7.083 (1.69), 7.096 (0.78), 7.186 (1.41), 7.197 (3.18), 7.212 (3.62), 7.236 (3.90), 7.251 (1.73), 7.273 (1.34), 7.286 (2.03), 7.300 (0.99), 7.349 (1.01), 7.365 (1.05), 7.607 (0.61), 7.622 (0.92), 7.627 (0.91), 7.634 (0.88), 7.641 (0.78), 7.730 (0.86), 7.744 (0.79), 8.291 (0.99), 8.304 (0.91), 8.678 (2.58), 8.683 (2.39), 8.710 (2.20), 9.790 (1.87).

Example C47: Preparation of trisodium 1-[(2S)-15-[(N²,N⁶-bis{14-[4-({[(1R)-2-carboxylato-1-{3-[({3-[(propylcarbamoyl)amino]phenyl}sulfonyl)amino]phenyl}ethyl]carbamoyl}amino)anilino]-14-oxo-4,7,10-trioxa-13-azatetradecan-1-oyl}-L-lysyl)amino]-2-(carboxylatomethyl)-4-oxo-7,10,13-trioxa-3-azapentadecan-1-oyl]-L-prolyl-N—[(R*)—{[16,20-difluoro-2,3,4,5-tetrahydro-12H-17,13-(azeno)-11,7-(metheno)-1,6,12,14-benzodioxadiazacyclononadecin-9-yl]methyl}(methyl)oxido-lambda⁶-sulfanylidene]-L-valinamide (Compound 47)

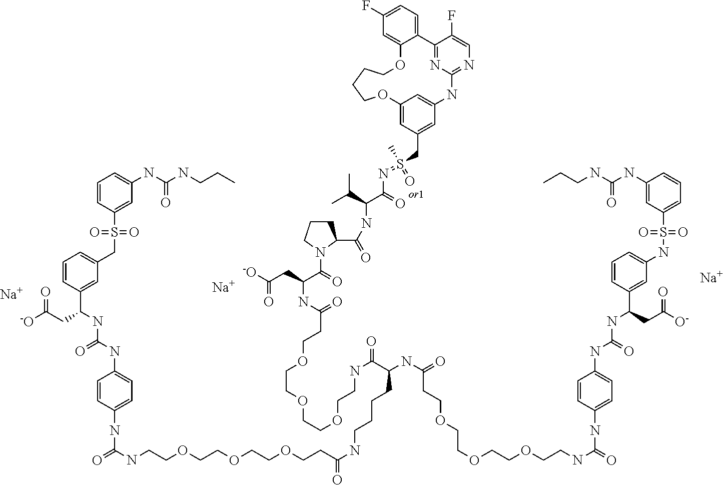

Compound 47 was synthesized analogously to Compound 26 but using Building block 15 instead. Compound 47 was obtained as a colorless foam (149 mg, 91.6% purity, 91% yield). LC-MS (Method 3): Rₜ=4.42 mi; MS (ESIpos): m/z=2669 [M+H]. ¹H-NMR (600 MHz, DMSO-d₆) δ[ppm]: 0.818 (3.76), 0.827 (4.25), 0.831 (7.23), 0.838 (2.38), 0.843 (3.61), 1.347 (0.66), 1.359 (1.30), 1.371 (2.01), 1.383 (1.89), 1.395 (0.98), 1.859 (1.03), 1.907 (0.54), 2.271 (0.70), 2.281 (1.40), 2.292 (0.75), 2.320 (0.66), 2.330 (0.73), 2.340 (0.47), 2.353 (0.42), 2.365 (0.67), 2.381 (0.88), 2.515 (0.72), 2.518 (0.69), 2.521 (0.61), 2.564 (0.59), 2.595 (0.71), 2.603 (0.78), 2.622 (0.44), 2.938 (0.68), 2.948 (1.47), 2.959 (1.51), 2.969 (0.80), 2.986 (0.61), 3.157 (0.53), 3.174 (4.08), 3.188 (0.64), 3.200 (0.96), 3.209 (2.01), 3.219 (2.06), 3.228 (0.89), 3.365 (1.42), 3.375 (1.74), 3.385 (0.91), 3.415 (1.74), 3.424 (2.93), 3.433 (1.67), 3.451 (1.98), 3.458 (2.44), 3.469 (3.11), 3.476 (6.50), 3.490 (2.62), 3.512 (8.54), 3.549 (0.80), 3.561 (2.44), 3.566 (16.00), 3.571 (2.81), 3.582 (1.87), 3.592 (0.71), 3.637 (0.58), 4.047 (0.42), 4.052 (0.42), 4.130 (0.54), 4.267 (0.78), 4.713 (1.37), 4.968 (0.64), 4.981 (0.60), 6.110 (0.60), 6.512 (0.95), 6.656 (0.94), 6.707 (0.58), 6.720 (0.59), 6.872 (0.51), 6.942 (1.05), 6.955 (1.18), 7.071 (0.89), 7.084 (1.43), 7.097 (0.67), 7.139 (0.62), 7.159 (0.66), 7.189 (2.90), 7.203 (3.85), 7.236 (3.11), 7.251 (1.57), 7.275 (1.09), 7.288 (1.65), 7.302 (0.81), 7.390 (0.43), 7.661 (0.57), 7.739 (0.45), 7.753 (0.42), 7.789 (0.61), 7.914 (0.64), 7.941 (0.61), 7.955 (0.63), 7.985 (0.92), 8.280 (0.44), 8.293 (0.40), 8.363 (1.17), 8.655 (1.10), 8.658 (1.05), 9.710 (0.90).

Example C48: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxido-lambda⁶-sulfanylidene]-L-valinamide (Compound 48)

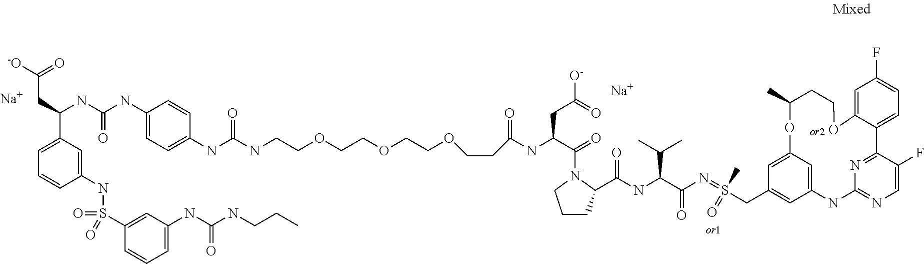

Compound 48 was synthesized using Building block 18 following the same general procedure as described previously to synthesize Compound 36. Compound 48 was obtained as an amorphous residue (39.60 mg, 100% purity, 97% yield). LC-MS (Method 3): Rₜ=4.43 min; MS (ESIpos): m/z=1554 [M+H]⁺. ¹H-NMR (600 MHz, DMSO-d₆) δ[ppm]: 0.814 (6.85), 0.823 (6.93), 0.827 (11.15), 0.839 (6.63), 0.842 (6.00), 0.854 (5.51), 1.233 (0.70), 1.346 (1.54), 1.358 (2.91), 1.370 (2.91), 1.382 (1.48), 1.394 (0.42), 1.424 (4.79), 1.434 (4.76), 1.703 (0.82), 1.722 (0.49), 1.871 (0.79), 1.886 (0.72), 1.900 (0.63), 1.955 (1.58), 1.967 (1.31), 2.097 (0.60), 2.109 (0.79), 2.118 (0.84), 2.129 (0.55), 2.299 (0.99), 2.345 (1.61), 2.370 (0.88), 2.381 (1.16), 2.423 (0.55), 2.515 (3.15), 2.518 (2.58), 2.521 (2.04), 2.590 (1.24), 2.612 (1.64), 2.652 (1.01), 2.667 (0.52), 2.915 (0.91), 2.926 (2.12), 2.937 (2.12), 2.948 (0.93), 3.093 (10.15), 3.145 (0.57), 3.195 (1.19), 3.205 (1.72), 3.215 (1.37), 3.415 (2.28), 3.424 (3.61), 3.433 (1.87), 3.457 (2.37), 3.463 (3.27), 3.481 (3.88), 3.488 (2.99), 3.495 (2.30), 3.512 (16.00), 3.551 (0.96), 3.566 (5.07), 3.580 (1.28), 3.591 (0.84), 3.661 (0.69), 3.743 (0.63), 4.062 (1.15), 4.072 (1.39), 4.077 (1.75), 4.086 (1.63), 4.375 (0.75), 4.443 (0.58), 4.462 (1.06), 4.487 (1.00), 4.496 (1.33), 4.506 (0.88), 4.684 (1.16), 4.707 (1.57), 4.837 (1.34), 4.859 (1.34), 4.873 (0.75), 4.884 (0.75), 4.967 (0.87), 6.530 (2.37), 6.656 (0.97), 6.669 (1.03), 6.743 (2.45), 6.902 (0.64), 6.916 (1.16), 6.930 (1.85), 6.944 (1.64), 7.052 (1.31), 7.065 (2.19), 7.078 (1.00), 7.172 (1.30), 7.186 (1.69), 7.206 (1.48), 7.221 (3.33), 7.236 (4.63), 7.251 (1.73), 7.274 (1.70), 7.287 (2.69), 7.301 (1.63), 7.341 (1.21), 7.357 (1.19), 7.556 (0.90), 7.611 (0.79), 7.806 (1.15), 8.125 (0.46), 8.248 (0.57), 8.706 (2.42), 8.711 (2.93), 8.716 (2.36), 8.791 (0.96), 9.743 (1.43), 9.906 (0.72).

Example C49: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[(4R*)-15,19-difluoro-4-methyl-3,4-dihydro-2H,11H-12,16-(azeno)-10,6-(metheno)-1,5,11,13-benzodioxadiazacyclooctadecin-8-yl]methyl}(methyl)oxido-lambda⁶-sulfanylidene]-L-valinamide (Compound 49)

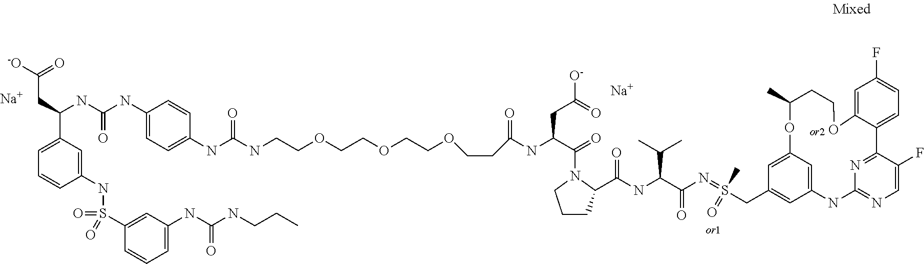

Compound 49 was synthesized using Building block 19 following the same general procedure as described previously to synthesize Compound 36. Compound 49 was obtained as an amorphous residue (38.10 mg, 100% purity, 96% yield). LC-MS (Method 3): Rₜ=4.43 min; MS (ESIpos): m/z=1554 [M+H]⁺. ¹H-NMR (600 MHz, DMSO-d₆) δ[ppm]: 0.815 (2.20), 0.820 (2.42), 0.827 (6.39), 0.831 (3.07), 0.839 (3.70), 1.347 (0.63), 1.359 (1.16), 1.371 (1.14), 1.383 (0.59), 1.426 (1.85), 1.436 (1.85), 1.849 (0.54), 1.931 (0.64), 1.941 (0.55), 2.338 (0.67), 2.352 (0.46), 2.363 (0.45), 2.515 (1.05), 2.518 (0.82), 2.521 (0.64), 2.591 (0.46), 2.612 (0.58), 2.927 (0.85), 2.938 (0.85), 3.200 (4.25), 3.214 (0.66), 3.414 (0.90), 3.424 (1.45), 3.433 (0.78), 3.441 (0.51), 3.449 (0.90), 3.453 (0.90), 3.460 (1.08), 3.478 (1.48), 3.485 (1.15), 3.510 (5.90), 3.555 (0.61), 3.566 (16.00), 3.575 (0.57), 3.675 (0.42), 4.062 (0.46), 4.072 (0.53), 4.077 (0.64), 4.086 (0.69), 4.466 (0.65), 4.475 (0.72), 4.485 (0.50), 4.696 (0.81), 4.704 (0.82), 6.485 (0.90), 6.657 (0.41), 6.669 (0.40), 6.754 (0.91), 6.917 (0.49), 6.920 (0.49), 6.931 (0.75), 6.945 (0.62), 7.053 (0.58), 7.066 (0.90), 7.079 (0.42), 7.173 (0.55), 7.186 (0.68), 7.206 (0.66), 7.221 (1.44), 7.237 (1.89), 7.252 (0.70), 7.274 (0.70), 7.287 (1.06), 7.301 (0.61), 7.341 (0.47), 7.361 (0.47), 7.365 (0.45), 8.693 (1.10), 8.697 (1.10), 8.705 (0.96), 9.787 (0.67).

Example C50: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[3,20-difluoro-13-oxa-5,7,18,25-tetraazatetracyclo[17.3.1.1^{2,6}.1^{8,12}]pentacosa-1(23),2(25),3,5,8(24),9,11,19,21-nonaen-10-yl]methyl}(methyl)oxido-lambda⁶-sulfanylidene]-L-valinamide (Compound 50)

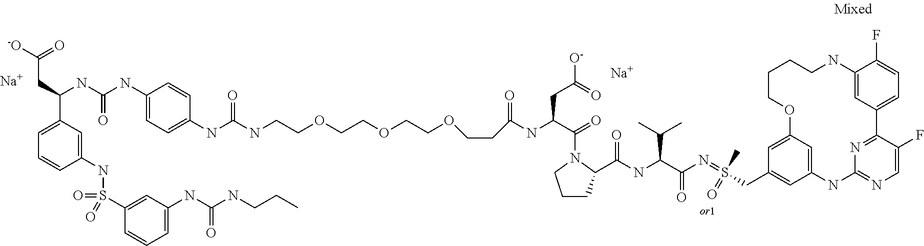

Compound 50 was synthesized using Building block 28 following the same general procedure as described previously to synthesize Compound 36. Compound 50 was obtained as a colorless foam (18.00 mg, 100% purity, 98% yield). LC-MS (Method 3): Rₜ=4.43 min; MS (ESIpos): m/z=1552 [M+H]⁺. ¹H-NMR (600 MHz, DMSO-d₆) δ[ppm]: 0.814 (6.59), 0.833 (11.08), 0.840 (5.47), 0.851 (5.99), 0.857 (4.91), 1.235 (0.71), 1.351 (1.31), 1.368 (2.41), 1.386 (2.34), 1.404 (1.23), 1.627 (1.27), 1.849 (1.07), 1.863 (1.31), 1.971 (1.50), 1.986 (1.20), 2.106 (0.53), 2.123 (0.74), 2.136 (0.75), 2.152 (0.55), 2.325 (1.73), 2.335 (1.76), 2.369 (0.72), 2.394 (0.65), 2.415 (0.69), 2.435 (0.62), 2.569 (1.01), 2.591 (1.05), 2.601 (1.05), 2.630 (0.49), 2.672 (0.40), 2.718 (0.69), 2.730 (0.81), 2.758 (0.68), 2.772 (0.58), 2.927 (0.91), 2.943 (1.93), 2.959 (1.93), 2.975 (0.87), 3.120 (9.07), 3.139 (0.94), 3.205 (3.02), 3.220 (3.00), 3.413 (3.13), 3.426 (4.00), 3.441 (2.42), 3.468 (3.98), 3.478 (4.01), 3.484 (2.96), 3.490 (2.97), 3.514 (16.00), 3.556 (1.69), 3.568 (4.18), 3.572 (3.16), 3.588 (1.54), 3.621 (0.68), 3.645 (0.78), 3.723 (0.66), 4.058 (0.98), 4.071 (1.02), 4.079 (1.00), 4.092 (1.00), 4.141 (1.64), 4.503 (0.79), 4.518 (1.37), 4.532 (0.75), 4.753 (1.05), 4.787 (1.28), 4.922 (1.80), 4.955 (1.23), 5.962 (0.94), 6.209 (0.42), 6.693 (0.88), 6.714 (0.94), 6.817 (2.21), 6.872 (2.08), 6.939 (1.20), 6.958 (1.50), 7.064 (1.31), 7.083 (2.01), 7.103 (0.92), 7.142 (0.97), 7.163 (1.50), 7.171 (1.30), 7.192 (3.38), 7.203 (2.57), 7.215 (4.59), 7.235 (4.98), 7.258 (2.39), 7.269 (2.24), 7.289 (2.42), 7.309 (1.08), 7.625 (1.34), 7.643 (1.93), 7.700 (0.58), 7.789 (1.14), 7.810 (1.05), 8.002 (0.42), 8.055 (2.41), 8.281 (1.13), 8.301 (1.10), 8.462 (0.55), 8.603 (2.45), 8.614 (2.27), 8.721 (0.87), 9.742 (2.24).

Example C51: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[3,20-difluoro-13-oxa-5,7,18,25-tetraazatetracyclo[17.3.1.1^{2,6}.1^{8,12}]pentacosa-1(23),2(25),3,5,8(24),9,11,19,21-nonaen-10-yl]methyl}(methyl)oxido-lambda⁶-sulfanylidene]-L-valinamide (Compound 51)

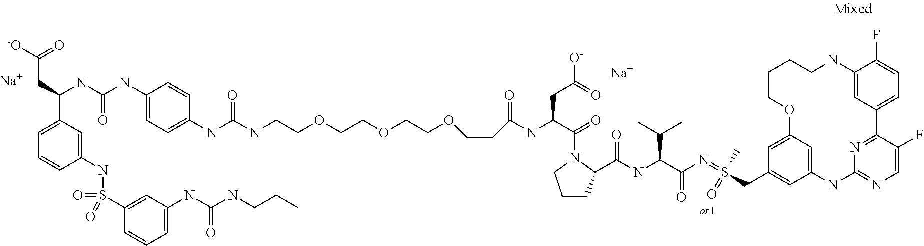

Compound 51 was synthesized using Building block 29 following the same general procedure as described previously to synthesize Compound 36. Compound 51 was obtained as a colorless foam (16.00 mg, 96% purity, 96% yield). LC-MS (Method 3): Rₜ=3.36 min; MS (ESIpos): m/z=1554 [M+H]⁺. ¹H-NMR (600 MHz, DMSO-d₆) δ[ppm]: 0.069 (0.63), 0.812 (6.78), 0.819 (6.21), 0.825 (6.94), 0.831 (11.81), 0.836 (6.00), 0.849 (5.10), 1.234 (0.61), 1.345 (1.38), 1.363 (2.54), 1.381 (2.48), 1.399 (1.28), 1.629 (1.36), 1.799 (1.12), 1.847 (1.37), 1.864 (1.17), 1.912 (0.98), 1.927 (1.49), 1.942 (1.08), 2.080 (0.57), 2.096 (0.78), 2.111 (0.78), 2.128 (0.52), 2.269 (0.45), 2.290 (0.61), 2.306 (1.09), 2.320 (1.12), 2.336 (1.48), 2.353 (0.90), 2.373 (0.73), 2.381 (0.77), 2.400 (0.66), 2.560 (1.00), 2.586 (1.03), 2.598 (1.06), 2.627 (0.52), 2.667 (0.82), 2.681 (0.80), 2.707 (0.63), 2.721 (0.53), 2.918 (0.94), 2.934 (1.96), 2.950 (1.99), 2.966 (0.90), 3.214 (3.68), 3.244 (10.55), 3.412 (3.89), 3.426 (4.63), 3.440 (3.10), 3.459 (3.37), 3.466 (3.99), 3.478 (3.78), 3.483 (3.39), 3.512 (16.00), 3.548 (1.26), 3.568 (5.42), 3.589 (1.07), 3.680 (1.56), 4.061 (1.02), 4.075 (1.10), 4.083 (1.05), 4.097 (1.02), 4.143 (2.04), 4.469 (0.83), 4.484 (1.29), 4.497 (0.78), 4.774 (3.12), 4.835 (0.40), 4.853 (0.86), 4.870 (0.87), 4.963 (0.70), 5.960 (0.95), 6.671 (1.07), 6.690 (1.11), 6.811 (2.11), 6.846 (2.19), 6.935 (1.20), 6.955 (1.55), 7.056 (1.48), 7.075 (2.24), 7.095 (0.98), 7.141 (0.96), 7.162 (1.36), 7.170 (1.18), 7.177 (1.44), 7.198 (3.30), 7.222 (4.72), 7.238 (5.87), 7.261 (2.77), 7.271 (2.75), 7.291 (2.58), 7.311 (1.17), 7.593 (1.47), 7.633 (1.12), 7.651 (1.10), 7.768 (1.22), 7.785 (1.14), 7.808 (0.95), 8.049 (2.52), 8.086 (0.66), 8.245 (1.02), 8.264 (1.02), 8.590 (3.04), 8.601 (2.68), 8.769 (1.44), 9.809 (2.03).

Example C52: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[(6R*)-17,21-difluoro-6-methyl-3,4,5,6-tetrahydro-2H-8,12-(azeno)-18,14-(metheno)-1,7,13,15-benzodioxadiazacycloicosin-10(13H)-yl]methyl}(methyl)oxido-lambda⁶-sulfanylidene]-L-valinamide (Compound 52)

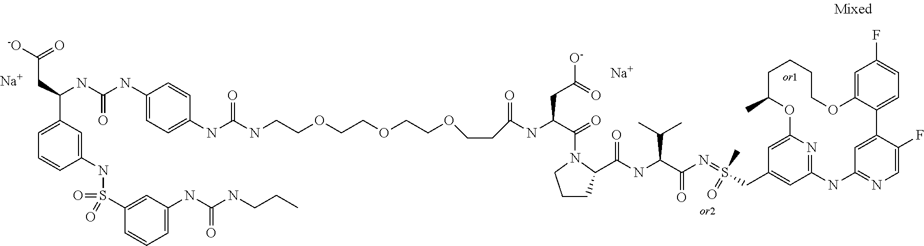

Compound 52 was synthesized using Building block 27 following the same general procedure as described previously to synthesize Compound 36. Compound 52 was obtained as a colorless foam (17.00 mg, 100% purity, 93% yield). LC-MS (Method 3): Rₜ=4.68 min; MS (ESIpos): m/z=1583 [M+H]⁺. ¹H-NMR (600 MHz, DMSO-d₆) δ[ppm]: 0.794 (2.62), 0.811 (4.95), 0.817 (3.46), 0.827 (2.89), 0.836 (6.01), 0.854 (3.08), 1.180 (2.80), 1.196 (2.77), 1.236 (0.48), 1.357 (1.05), 1.375 (1.59), 1.393 (1.50), 1.411 (0.90), 1.608 (0.48), 1.700 (0.60), 1.847 (0.52), 1.906 (0.89), 2.063 (0.43), 2.077 (0.44), 2.319 (0.90), 2.330 (1.11), 2.346 (0.41), 2.367 (0.54), 2.384 (0.41), 2.406 (0.43), 2.425 (0.42), 2.525 (0.83), 2.565 (0.60), 2.580 (0.49), 2.594 (0.55), 2.608 (0.58), 2.654 (0.47), 2.668 (0.64), 2.711 (0.47), 2.938 (0.47), 2.955 (0.99), 2.970 (1.00), 2.986 (0.49), 3.208 (1.16), 3.222 (1.28), 3.235 (0.69), 3.284 (6.33), 3.414 (1.58), 3.428 (2.23), 3.442 (1.44), 3.461 (1.64), 3.467 (2.18), 3.478 (2.20), 3.484 (1.58), 3.504 (1.37), 3.516 (9.73), 3.552 (1.03), 3.568 (16.00), 3.585 (0.91), 3.638 (0.90), 4.047 (0.59), 4.061 (0.62), 4.069 (0.67), 4.083 (0.79), 4.467 (0.44), 4.481 (0.75), 4.494 (0.42), 4.737 (1.00), 4.755 (0.95), 4.833 (0.41), 4.847 (0.71), 4.988 (0.43), 6.273 (1.88), 6.633 (1.64), 6.725 (0.42), 6.743 (0.46), 6.874 (0.41), 6.889 (0.73), 6.896 (0.80), 6.910 (0.41), 6.916 (0.47), 6.943 (0.67), 6.963 (0.81), 7.075 (0.79), 7.094 (1.24), 7.114 (0.62), 7.174 (0.70), 7.179 (0.81), 7.190 (1.54), 7.213 (3.46), 7.229 (2.87), 7.252 (0.86), 7.267 (1.01), 7.287 (1.32), 7.307 (0.68), 7.318 (0.42), 7.340 (0.74), 7.357 (0.41), 7.715 (0.87), 7.736 (0.68), 8.181 (1.21), 8.196 (1.17), 8.299 (2.13), 8.303 (1.90), 8.315 (0.67), 8.420 (0.43), 9.688 (1.38).

Example C53: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[(6S*)-17,21-difluoro-6-methyl-3,4,5,6-tetrahydro-2H-8,12-(azeno)-18,14-(metheno)-1,7,13,15-benzodioxadiazacycloicosin-10(13H)-yl]methyl}(methyl)oxido-lambda⁶-sulfanylidene]-L-alaninamide (Compound 53)

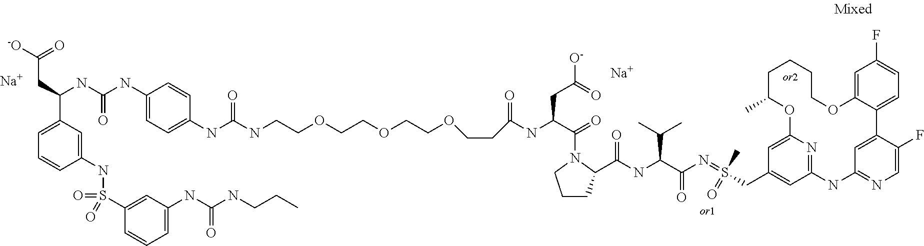

Compound 53 was synthesized using Building block 24 following the same general procedure as described previously to synthesize Compound 36. Compound 53 was obtained as an amorphous residue (11.00 mg, 94% purity, 72% yield). LC-MS (Method 3): Rₜ=4.54 min; MS (ESIpos): m/z=1554 [M+H]⁺. ¹H-NMR (600 MHz, DMSO-d₆) δ[ppm]: 0.815 (5.87), 0.827 (12.46), 0.839 (6.20), 1.184 (7.74), 1.194 (7.74), 1.227 (6.95), 1.239 (6.94), 1.266 (0.69), 1.278 (0.64), 1.334 (0.62), 1.346 (2.32), 1.358 (4.50), 1.370 (4.44), 1.382 (2.57), 1.432 (0.72), 1.446 (0.83), 1.454 (0.94), 1.598 (1.42), 1.624 (0.91), 1.694 (1.71), 1.861 (1.45), 1.873 (1.24), 1.967 (2.18), 1.979 (1.82), 2.251 (0.65), 2.276 (1.01), 2.348 (0.69), 2.359 (1.01), 2.371 (0.87), 2.383 (1.31), 2.425 (0.47), 2.516 (2.31), 2.519 (2.25), 2.523 (2.27), 2.526 (2.14), 2.587 (1.55), 2.614 (1.56), 2.647 (0.88), 2.654 (0.98), 2.659 (0.97), 2.673 (0.84), 2.685 (0.73), 2.913 (1.30), 2.924 (3.03), 2.935 (3.03), 2.946 (1.28), 3.163 (13.24), 3.182 (1.70), 3.197 (2.09), 3.204 (2.56), 3.212 (2.11), 3.421 (3.12), 3.430 (4.73), 3.439 (2.71), 3.447 (1.84), 3.456 (3.45), 3.463 (3.87), 3.470 (2.89), 3.487 (5.36), 3.495 (4.78), 3.517 (16.00), 3.532 (1.49), 3.542 (1.45), 3.547 (1.44), 3.558 (1.80), 3.568 (7.10), 3.581 (0.86), 3.592 (1.45), 3.604 (1.16), 3.717 (0.84), 3.832 (0.75), 4.079 (1.80), 4.091 (2.75), 4.103 (2.10), 4.225 (1.13), 4.381 (1.23), 4.390 (2.13), 4.399 (1.23), 4.756 (1.52), 4.778 (1.96), 4.833 (1.05), 4.845 (1.20), 4.854 (1.22), 4.865 (1.59), 4.876 (2.60), 4.899 (1.31), 4.967 (1.22), 6.299 (4.61), 6.651 (4.24), 6.864 (0.84), 6.868 (0.93), 6.878 (1.67), 6.882 (1.74), 6.892 (1.04), 6.931 (1.89), 6.944 (2.27), 7.048 (1.84), 7.061 (3.11), 7.075 (1.40), 7.172 (3.54), 7.186 (3.56), 7.234 (14.72), 7.250 (1.12), 7.276 (2.10), 7.289 (3.19), 7.303 (2.31), 7.312 (1.80), 7.326 (2.43), 7.338 (1.17), 7.532 (1.85), 7.839 (1.59), 8.165 (1.59), 8.193 (3.15), 8.202 (3.05), 8.275 (0.77), 8.336 (4.41), 8.695 (0.40), 8.806 (1.98), 9.894 (0.80).

Example C54: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[(6S*)-17,21-difluoro-6-methyl-3,4,5,6-tetrahydro-2H-8,12-(azeno)-18,14-(metheno)-1,7,13,15-benzodioxadiazacycloicosin-10(13H)-yl]methyl}(methyl)oxido-lambda⁶-sulfanylidene]-L-valinamide (Compound 54)

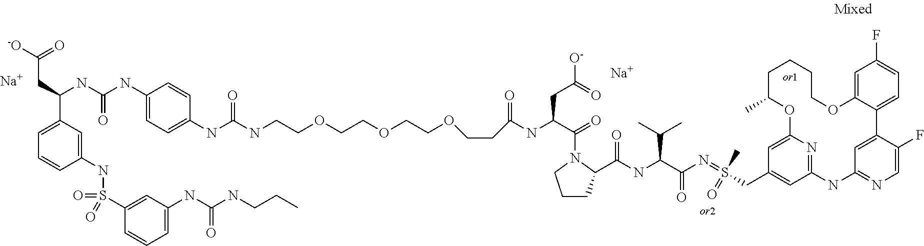

Compound 54 was synthesized using Building block 24 following the same general procedure as described previously to synthesize Compound 36. Compound 54 was obtained as an amorphous residue (15.00 mg, 96% purity, 82% yield). LC-MS (Method 3): Rₜ=4.69 min; MS (ESIpos): m/z=1582 [M+H]⁺. ¹H-NMR (600 MHz, DMSO-d₆) δ[ppm]: 0.800 (6.75), 0.815 (10.57), 0.827 (16.00), 0.839 (6.27), 1.184 (7.07), 1.195 (7.07), 1.235 (0.77), 1.345 (2.12), 1.357 (4.08), 1.369 (4.08), 1.381 (2.47), 1.454 (0.93), 1.596 (1.38), 1.694 (1.64), 1.874 (1.77), 1.947 (1.80), 1.958 (1.80), 2.096 (0.77), 2.107 (1.09), 2.117 (1.06), 2.267 (0.71), 2.358 (0.96), 2.370 (0.84), 2.385 (1.64), 2.425 (0.67), 2.588 (1.77), 2.614 (2.63), 2.653 (0.96), 2.912 (1.29), 2.922 (2.80), 2.934 (2.86), 2.944 (1.22), 3.189 (13.62), 3.206 (2.22), 3.417 (3.02), 3.426 (4.31), 3.435 (2.63), 3.442 (1.86), 3.451 (3.24), 3.459 (3.79), 3.465 (2.89), 3.483 (4.56), 3.492 (4.53), 3.512 (15.55), 3.550 (1.67), 3.568 (5.69), 3.593 (1.38), 3.709 (0.87), 3.778 (0.74), 4.023 (1.03), 4.035 (1.19), 4.046 (1.00), 4.096 (1.22), 4.226 (1.12), 4.476 (1.35), 4.766 (1.38), 4.788 (1.83), 4.853 (3.41), 4.875 (2.02), 4.967 (1.16), 6.323 (4.82), 6.640 (3.86), 6.657 (1.70), 6.871 (0.87), 6.885 (1.67), 6.896 (0.90), 6.930 (1.70), 6.944 (2.12), 7.048 (1.64), 7.061 (2.83), 7.073 (1.25), 7.171 (3.28), 7.183 (2.54), 7.190 (1.86), 7.233 (13.69), 7.276 (1.86), 7.290 (2.89), 7.303 (1.86), 7.317 (1.86), 7.329 (2.47), 7.527 (1.77), 7.846 (1.93), 8.171 (1.03), 8.193 (3.34), 8.202 (3.31), 8.323 (4.14), 8.810 (1.80), 9.738 (0.64), 9.889 (0.87).

Example C55: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[(6S*)-17,21-difluoro-6-methyl-3,4,5,6-tetrahydro-2H-8,12-(azeno)-18,14-(metheno)-1,7,13,15-benzodioxadiazacycloicosin-10(13H)-yl]methyl}(methyl)oxido-lambda⁶-sulfanylidene]-L-valinamide (Compound 55)

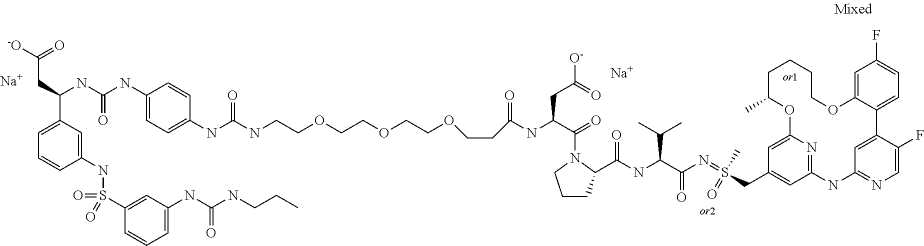

Compound 55 was synthesized using Building block 25 following the same general procedure as described previously to synthesize Compound 36. Compound 55 was obtained as an amorphous residue (16.00 mg, 94% purity, 88% yield).

LC-MS (Method 3): Rₜ=4.69 min; MS (ESIpos): m/z=1582 [M+H]⁺

¹H-NMR (600 MHz, DMSO-d₆) δ[ppm]: 0.777 (7.55), 0.787 (12.70), 0.798 (7.55), 0.814 (5.77), 0.826 (12.43), 0.838 (6.52), 1.188 (8.03), 1.198 (7.97), 1.235 (0.62), 1.331 (0.55), 1.343 (2.20), 1.354 (4.74), 1.366 (4.74), 1.379 (2.95), 1.390 (1.37), 1.445 (0.89), 1.454 (0.96), 1.595 (1.44), 1.684 (1.65), 1.694 (1.85), 1.706 (1.44), 1.841 (0.82), 1.902 (1.03), 1.919 (1.24), 1.931 (0.89), 1.960 (1.10), 2.110 (0.89), 2.121 (1.30), 2.132 (1.30), 2.143 (0.82), 2.174 (1.10), 2.185 (1.44), 2.197 (1.99), 2.212 (1.37), 2.223 (0.96), 2.385 (2.06), 2.401 (1.17), 2.415 (0.89), 2.424 (1.44), 2.613 (1.44), 2.653 (0.82), 2.731 (0.76), 2.890 (0.89), 2.907 (1.24), 2.917 (3.02), 2.928 (2.95), 2.939 (1.17), 3.152 (1.17), 3.162 (1.10), 3.199 (1.24), 3.209 (1.30), 3.235 (1.03), 3.300 (16.00), 3.407 (2.47), 3.416 (3.91), 3.427 (2.88), 3.435 (1.99), 3.444 (1.92), 3.463 (2.47), 3.478 (4.46), 3.486 (6.32), 3.492 (9.00), 3.500 (8.38), 3.512 (3.30), 3.518 (2.40), 3.567 (4.46), 3.611 (0.76), 3.626 (1.24), 3.634 (1.10), 3.795 (0.89), 3.864 (1.10), 4.042 (1.37), 4.053 (1.65), 4.057 (1.65), 4.068 (1.44), 4.096 (1.24), 4.107 (1.03), 4.224 (1.10), 4.469 (1.44), 4.482 (1.51), 4.700 (0.62), 4.724 (3.50), 4.751 (1.24), 4.762 (1.79), 4.775 (1.79), 4.826 (0.82), 4.837 (1.44), 4.847 (1.44), 4.949 (0.69), 6.295 (5.29), 6.701 (4.53), 6.873 (1.17), 6.884 (1.99), 6.887 (1.92), 6.901 (1.10), 7.171 (2.47), 7.190 (2.47), 7.209 (3.85), 7.224 (5.63), 7.253 (4.46), 7.267 (2.54), 7.319 (1.58), 7.330 (2.40), 7.342 (1.37), 7.502 (0.62), 7.871 (0.48), 8.012 (1.79), 8.027 (1.58), 8.068 (1.85), 8.080 (1.79), 8.192 (3.36), 8.201 (3.23), 8.315 (4.67), 8.812 (0.62), 9.897 (3.16).

Example C56: Preparation of disodium 1-{(2S)-2-(carboxylatomethyl)-17-[4-({[(1R)-2-carboxylato-1-{3-[({3-[(propylcarbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-N—[(R*)—{[(6R*)-17,21-difluoro-6-methyl-3,4,5,6-tetrahydro-2H-8,12-(azeno)-18,14-(metheno)-1,7,13,15-benzodioxadiazacycloicosin-10(13H)-yl]methyl}(methyl)oxido-lambda⁶-Fcx35 ] sulfanylidene]-L-valinamide (Compound 56)

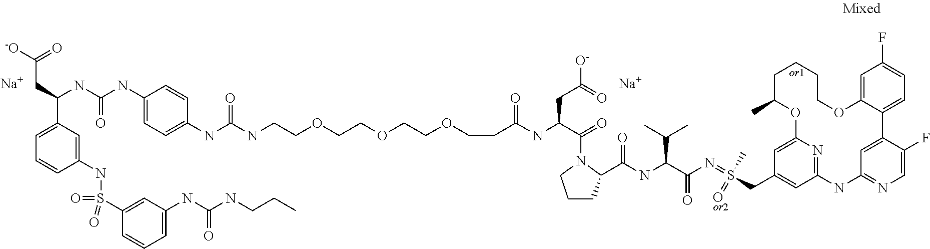

Compound 56 was synthesized using Building block 26 following the same general procedure as described previously to synthesize Compound 36. Compound 56 was obtained as a colorless foam (15.00 mg, 100% purity, 97% yield). LC-MS (Method 5): Rₜ=1.07 min; MS (ESIpos): m/z=1582 [M+H]⁺. ¹H-NMR (600 MHz, DMSO-d₆) δ[ppm]: 0.068 (0.61), 0.805 (6.08), 0.816 (10.90), 0.823 (7.16), 0.828 (12.29), 0.835 (6.43), 0.840 (6.56), 0.870 (0.48), 0.881 (0.43), 1.184 (6.52), 1.195 (6.24), 1.235 (0.55), 1.335 (0.55), 1.347 (2.09), 1.359 (3.81), 1.371 (3.85), 1.383 (2.02), 1.395 (0.70), 1.438 (0.75), 1.448 (0.83), 1.600 (1.39), 1.687 (1.24), 1.701 (1.22), 1.711 (0.97), 1.864 (1.31), 1.875 (1.34), 1.888 (0.84), 1.930 (0.71), 1.944 (1.66), 1.955 (1.63), 2.092 (0.68), 2.103 (1.03), 2.114 (0.99), 2.125 (0.70), 2.258 (0.60), 2.271 (0.62), 2.282 (0.88), 2.341 (1.09), 2.352 (1.51), 2.363 (0.98), 2.377 (0.80), 2.386 (0.90), 2.589 (1.42), 2.617 (1.48), 2.653 (0.80), 2.916 (1.15), 2.926 (2.58), 2.937 (2.63), 2.948 (1.07), 3.171 (1.16), 3.188 (12.16), 3.203 (1.95), 3.216 (1.58), 3.239 (0.55), 3.419 (2.64), 3.428 (4.14), 3.437 (2.34), 3.445 (1.68), 3.453 (2.93), 3.461 (3.13), 3.484 (4.27), 3.492 (4.22), 3.515 (16.00), 3.539 (1.19), 3.545 (1.15), 3.555 (1.59), 3.568 (9.66), 3.586 (1.41), 3.597 (1.07), 3.612 (0.48), 3.686 (0.77), 3.700 (0.86), 3.747 (0.74), 4.023 (1.02), 4.033 (1.22), 4.047 (1.00), 4.088 (0.80), 4.095 (1.08), 4.231 (0.94), 4.483 (1.02), 4.491 (1.28), 4.501 (0.99), 4.763 (1.34), 4.786 (1.70), 4.813 (0.74), 4.823 (1.43), 4.850 (2.84), 4.872 (1.38), 4.970 (1.05), 6.323 (4.35), 6.631 (3.40), 6.651 (1.48), 6.663 (1.53), 6.877 (0.79), 6.887 (1.44), 6.891 (1.54), 6.904 (0.82), 6.933 (1.68), 6.945 (1.98), 7.050 (1.59), 7.063 (2.60), 7.076 (1.19), 7.176 (2.00), 7.185 (2.49), 7.201 (1.74), 7.212 (1.61), 7.228 (5.59), 7.235 (6.90), 7.250 (1.54), 7.276 (1.73), 7.290 (2.77), 7.303 (2.07), 7.320 (1.48), 7.335 (1.72), 7.346 (0.96), 7.542 (1.78), 7.830 (1.56), 7.851 (1.00), 7.865 (0.88), 8.153 (0.86), 8.198 (2.47), 8.207 (2.63), 8.315 (3.73), 8.802 (1.77), 9.747 (1.10).

BIOLOGICAL EXAMPLES

Example B1: Cytotoxicity In Vitro in the Presence (+) and Absence (−) of Elastase

Cultivation of cells was performed according to standard procedures with the media recommended by the provider. The cells, in a total volume of 100 μL, were seeded in a 96-well plate with a white bottom (#3610). After a 24 h incubation period at 37° C. and 500 CO₂, the medium was exchanged by adding 90 μL fresh medium. The treatment started by adding the test compound in 10 μL of culture medium to the cells in triplicates. Concentrations ranging from 10⁻⁵ M to 10⁻¹³ M were chosen. Two identically treated sets of samples were prepared. One set was treated with the test compound alone, whereas to the second set the compound and 10 nM elastase was added followed by a 72 h incubation period at 37° C. and 50% CO₂. The proliferation was detected using the MTT assay (ATCC). At the end of the incubation period the MTT reagent was added to all samples for 4 h. Lysis of the cells followed by addition of the detergent was done overnight. The formed dye was detected at 570 nm. The proliferation of untreated but otherwise identically handled cells was define as the 100% value. The dose response curves allowed the determination of the respective IC₅₀ values, which are summarized in Table B1.

TABLE B1
Summary of IC₅₀ Values With (+) or
Without (−) Neutrophil Elastase

| | | | | | NCI | | | |
|---|---|---|---|---|---|---|---|---|
| | 786-O | | HT29 | | H292 | | SUM149 | |
| | IC₅₀ | | IC₅₀ | | IC₅₀ | | IC₅₀ | |
| Compound | − | + | − | + | − | + | − | + |
| 1 | C | C | D | C | D | C | D | C |
| 2 | D | C | D | B | D | C | D | C |
| 3 | D | A | D | A | D | A | D | A |
| 4 | D | C | D | C | D | C | D | C |
| 5 | D | A | D | A | D | B | D | B |
| 6 | n.t. | n.t. | D | A | D | A | C | A |
| 9 | D | B | D | C | D | C | D | C |
| 10 | D | A | C | A | D | A | D | B |
| 11 | n.t. | n.t. | C | A | D | A | C | A |
| 14 | D | A | D | A | D | B | D | A |
| 15 | D | A | D | A | C | A | C | A |
| 16 | D | B | D | A | C | A | C | B |
| 17 | D | B | C | A | C | A | C | B |
| 18 | D | B | C | A | D | B | C | A |
| 19 | D | B | C | A | C | B | B | A |

A ≤ 1 nM
B = 1.1-10 nM
C = 11-100 nM
D > 100 nM
n.t. = not tested

The cytotoxicity assay (Version 2) was performed as described above. Some changes were applied as the assays were performed in an automated fashion and the IC₅₀ values determination was changed. Here the change in metabolic activity of the cells, in percent, was calculated by normalization of the measured values to the absorbance values of medium containing wells without cells (═−100%) and the absorbance of the DMSO-treated cells (═0%). AC₅₀ values (activity concentration at −50%) were determined by means of a 4 parameter Hill-fit using GeneData Screener software. Values are summarized in Table B1b.

TABLE B1b
Summary of AC₅₀ Values With (+) or
Without (−) Neutrophil Elastase

| | | | | | NCI | | | |
|---|---|---|---|---|---|---|---|---|
| | 786-O | | HT29 | | H292 | | SUM149 | |
| | AC₅₀ | | AC₅₀ | | AC₅₀ | | AC₅₀ | |
| Compound | − | + | − | + | − | + | − | + |
| 1 | D | D | D | D | D | B | D | C |
| 2 | C | C | C | C | D | C | C | C |
| 3 | D | B | D | B | n.t. | B | C | B |
| 4 | D | B | D | C | D | D | D | C |
| 5 | D | B | D | B | C | B | D | B |
| 6 | A | A | A | A | C | A | A | A |
| 7 | B | B | B | B | C | C | B | B |
| 8 | D | C | D | B | C | C | D | C |
| 9 | D | B | D | B | D | B | D | B |
| 10 | D | A | D | A | C | B | C | A |
| 11 | C | C | C | C | C | S | C | C |
| 12 | C | A | D | A | C | C | C | A |
| 13 | D | B | D | B | C | C | D | B |
| 14 | D | B | D | B | C | B | C | B |
| 15 | C | C | C | C | C | B | C | C |
| 16 | D | B | D | A | C | B | D | B |
| 17 | C | A | D | A | C | A | C | A |
| 18 | D | B | D | B | C | B | C | A |
| 19 | C | C | C | C | C | B | C | C |
| 20 | D | B | D | B | D | B | C | B |
| 21 | D | B | D | B | C | B | C | B |
| 22 | C | A | C | A | C | A | C | A |
| 23 | C | A | C | A | C | A | C | A |
| 24 | C | A | C | A | C | A | C | A |
| 25 | D | B | D | B | n.t. | B | C | B |
| 28 | D | A | C | A | C | A | C | A |
| 29 | D | A | C | A | C | A | C | A |
| 30 | D | A | D | A | C | A | C | A |
| 31 | D | A | D | A | C | A | C | A |
| 32 | C | A | D | A | C | A | C | A |
| 33 | D | A | D | A | C | A | C | A |
| 36 | C | B | C | B | C | B | C | B |
| 37 | C | A | C | A | C | A | C | A |
| 38 | C | A | C | A | C | A | C | A |
| 39 | C | A | C | A | C | A | C | A |
| 40 | C | B | C | B | C | B | C | B |
| 41 | C | A | C | A | C | A | C | A |
| 42 | C | A | D | A | C | A | C | A |
| 43 | D | B | D | B | D | B | D | B |
| 44 | D | B | D | B | D | B | D | B |
| 45 | D | C | D | C | D | C | D | B |
| 46 | D | D | D | D | D | D | D | C |
| 47 | D | B | D | B | C | B | D | B |
| 48 | D | A | C | A | C | A | C | A |
| 49 | D | A | D | A | C | A | C | A |
| 50 | D | A | D | A | C | A | C | A |
| 51 | D | A | D | A | C | A | C | A |
| 52 | D | B | D | B | D | B | D | B |
| 53 | D | B | D | B | D | B | C | B |
| 54 | D | B | D | B | D | B | D | B |
| 55 | D | B | D | B | D | B | D | A |
| 56 | D | B | D | B | D | B | C | B |

A ≤ 10 nM
B = 11-100 nM
C = 0.1-1 μM
D > 1 μM
n.t. = not tested

Example B2: Cytotoxicity In Vitro in the Presence (+) and Absence (−) of Cathepsin B

Using the same assay design for examples with cathepsin B cleavable linker reveals that addition of cathepsin B allows no optimal generation of the payload due to cell culture conditions. The pH value for the cytotoxicity assay differs significantly from the pH optimum of cathepsin B, which is reflected in the minor improvement of the measured cytotoxic potency in Table B2.

TABLE B2
Summary of IC₅₀ Values With (+) or Without (−) Cathepsin

| | | | | | NCI | | | |
|---|---|---|---|---|---|---|---|---|
| | 786-O | | HT29 | | H292 | | SUM149 | |
| | IC₅₀ | | IC₅₀ | | IC₅₀ | | IC₅₀ | |
| Compound | − | + | − | + | − | + | − | + |
| 7 | n.t. | n.t. | D | D | C | C | D | C |
| 12 | n.t. | n.t. | D | D | D | D | C | C |

A ≤ 1 nM
B = 1.1-10 nM
C = 11-100 nM
D > 100 nM
n.t. = not tested

Example B3: Cytotoxicity In Vitro in the Presence (+) and Absence (−) of Legumain

Using the same assay design for examples with legumain cleavable linker reveals that addition of legumain allows no optimal generation of the payload due to cell culture conditions. The pH value for the cytotoxicity assay differs significantly from the pH optimum of legumain, which is reflected in the minor improvement of the measured cytotoxic potency in Table B3.

TABLE B3
Summary of IC₅₀ Values With (+) or Without (−) Legumain

| | | | | | NCI | | | |
|---|---|---|---|---|---|---|---|---|
| | 786-O | | HT29 | | H292 | | SUM149 | |
| Compound | IC₅₀ | | IC₅₀ | | IC₅₀ | | IC₅₀ | |
| legumain | − | + | − | + | − | + | − | + |
| 8 | n.t. | n.t. | C | C | C | B | C | C |
| 13 | n.t. | n.t. | C | C | C | C | C | C |

A < 1 nM
B = 1.1-10 nM
C = 11-100 nM
D > 100 nM
n.t. = not tested

Example B4: Biochemical Legumain Cleavage Assay and Cathepsin Cleavage Assay

### Cathepsin B Assay

Incubation of samples starts with addition of cathepsin B solution (2.5 μg in 250 ml 50 mM sodium phosphate buffer, pH 6.0/2 mM DTT). The incubation is performed at 40° C., and after 16 h incubation a 40 μL sample is taken and added to 80 μL ice-cold methanol. For the untreated control (0 h) ice-cold methanol is pipetted in a tube before the cathepsin B and compound solution are added. Samples can be stored at −20° C. or analyzed directly by HPLC. The metabolite (product) and the parent compound (educt) is measured.

### Legumain Assay

10 μg rec. human legumain are added to 100 μL activation buffer (50 mM sodium acetate buffer/100 mM NaCl, pH 4,0) and incubated for 2 h at 37° C., before 4.9 mL assay buffer (50 mM MES buffer/250 mM NaCl, pH 5,0) is added (final concentration of rec. hum. legumain: 2 μg/mL). For the selected sample, activated legumain is added to the compound solution. Then the solution is incubated at 37° C. At 16 h-a 50 μL sample is taken and added to 100 μL ice-cold methanol. For the untreated control (0 h) ice-cold methanol is pipetted in a tube before the legumain and compound solution are added. Samples can be stored at −20° C. or analyzed directly by HPLC or LCMS. The metabolite and the parent compound (educt) are measured.

### RP-HPLC Analysis

Samples from enzymatic assays are added to the double volume of methanol and incubated for at least 30 min at −20° C. to precipitate proteins. Samples are then centrifuged for 10 min, 16100×g at 4° C. and analyzed by RP-HPLC using a gradient (PLRP-S-A1 79.0% PLRP-S—B1 21.0%, duration 24 min). After the centrifugation the supernatant is collected, calibration curves are obtained using the respective compound and the corresponding metabolite solution. A 10 mM DMSO stock solution is diluted with Methanol to achieve the chosen end concentration.

### Metabolite Detection

Example C7 and C12 were evaluated in the cathepsin B assay described herein. As illustrated in FIG. 4, without addition of active cathepsin B, no metabolite formation is detected for either Example C7 or C12 (i.e., educt example C₇). The formation of metabolite after 16 h incubation with cathepsin B is shown in FIG. 4 (right). After incubation, 16% of the metabolite was detected for compound C7, and 24% metabolite was detected for compound C12 (data not shown).

Compounds C8 and C13 were evaluated in the legumain assay described herein. As illustrated in FIG. 5, without addition of active legumain, no metabolite formation is detected for either Compound C8 or C13 data not shown) (i.e., educt examples C₈). The formation of metabolite after 16 h incubation with legumain is shown in FIG. 5. After incubation, complete metabolite formation was detected for both compounds.

Example B6: αᵥβ₃ Binding Assay

Binding affinity of the respective αᵥβ₃ inhibitor is tested in an ELISA-like manner. 96-well plates are coated with vitronectin (1 μg/ml) or fibronectin 0,5 μg/ml) and afterwards uncoated surface space is blocked with BSA. After a 2 h pre-incubation of a serial dilution of the antagonist of interest (starting at 20 μM in a 1:5 dilution) with the extracellular domain of the integrin (2 μg/ml, αvβ3 or α5β1), the solution is added to the coated plates. After an 1 h incubation at RT and several washing steps, an integrin specific antibody is added for 1 h at RT. After three washing steps the secondary peroxidase-labeled antibody is added to the plate. After an 1 h incubation at RT the plate is developed by quick addition of SeramunBlau (50 μL per well, Seramun Diagnostic GmbH, Heidesee, Germany) and incubated for 5 min at RT in the dark. The reaction is stopped with 0.3M H₂SO₄ (50 μL/well), and the absorbance is measured at 450 nm with a plate reader. IC₅₀ of each compound is tested in duplicate, and the resulting inhibition curves are analyzed. As a reference standard (Ref-1), cilengitide is used. The αᵥβ₃ binding is used as 100% value.

Example B7: Compound Stability Assays in Buffer and Plasma

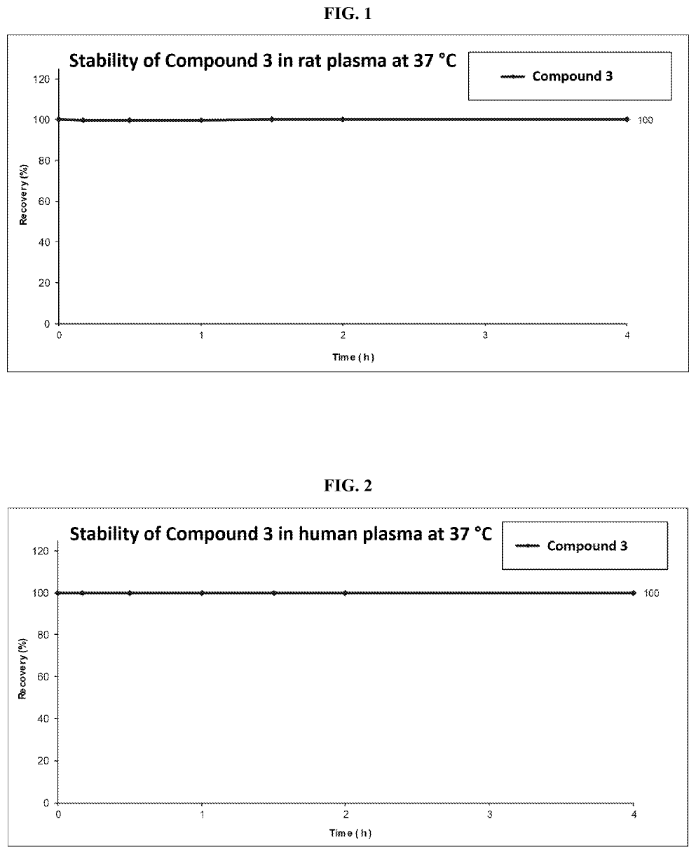

Method for measurement of stability in rat and human plasma: 1 mg of the test compound was dissolved in 0.5 ml acetonitrile/dimethylsulfoxide 1:1. For complete dissolution the HPLC vial was shaken and sonicated. While vortexing, 20 μl of this solution was added to 1 ml 37° C. warm plasma. After 10 min, 0.5 h, 1 h, 1.5 h, 2 h, and 4 h, the assay was stopped by adding 100 μl of the compound plasma solution to a vial containing 300 μl acetonitrile/buffer pH 3 (80:20) at RT. The mixture was centrifuged at 5000 rpm for 10 minutes. The supernatant was analyzed by HPLC to determine the amount of the test compound and up to two byproducts. To values resulted from a processed sample immediately taken after vortexing with plasma at RT. The peak areas (in percentage) were used for quantification. All data was given as percent area of the initial compound at to. The results of these assays are shown in FIG. 1 and FIG. 2 for rat and human plasma, respectively.

Method for measurement of stability in buffer: 0.15 mg of the test compound was dissolved in 0.1 ml dimethylsulfoxide and 0.4 ml acetonitrile. For complete dissolution, the HPLC vial with the sample solution was shaken and sonicated. Then 1.0 ml of the respective buffer solution was added, and the sample was vortexed. The sample solution was analysed by HPLC to determine the amount of the test compound and up to two byproducts at a particular time over a period of 24 h at 37° C. To values resulted from a sample immediately taken after vortexing with buffer at RT. The peak areas (in percentage) were used for quantification.

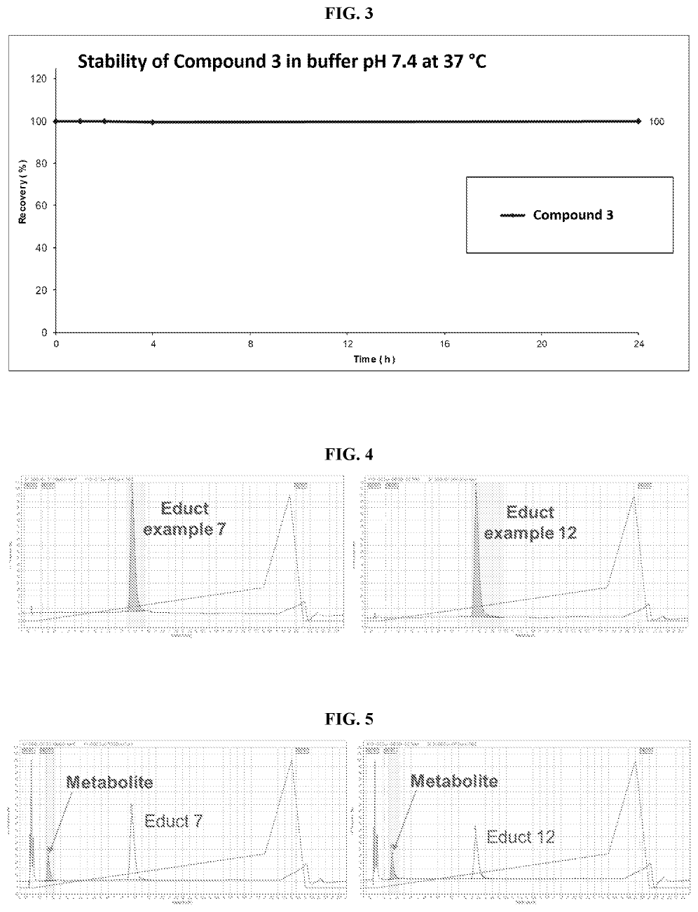

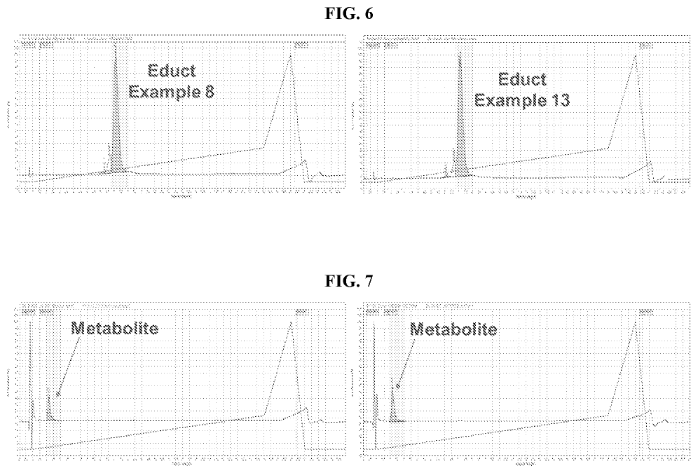

Compound 3 was evaluated for stability in rat plasma (FIG. 1), human plasma (FIG. 2), and buffer at pH 7.4 (FIG. 3), as described below. As indicated in FIGS. 1-3, Compound 3 is stable in plasma for at least 4 h, and in buffer for at least 24 h, with no significant degradation of test compound observed

Example B8: Pharmacokinetics

Male rats (n=3) were given an intravenous 0.5 or 2.0 mg/kg bolus dose of test article dissolved in plasma with 1% DMSO and 2% ethanol (C30 only). Serial blood samples were collected up to 24 hours post-dose. Plasma was harvested from blood samples and stored frozen until sample analysis. Test article plasma concentrations were analyzed by LC/MS/MS. Pharmacokinetic parameters were calculated using non-compartmental analysis and are presented in Table B8.

TABLE B8
Pharmacokinetics in rat after intravenous
administration of of test articles

| | IV Dose | CL | Vss | AUC | t_{1/2} |
|---|---|---|---|---|---|
| Compound | (mg/kg) | (mL/min/kg) | (L/kg) | (mg · h/L) | (h) |
| C41 | 2.0 | B | B | C* | A |
| C33 | 2.0 | B | B | A* | A |
| C48 | 0.5 | C | A | C | C |
| C30 | 0.5 | A | B | A | A |

*Dosed normalized to 0.5 mg/kg IV from a 2 mg/kg IV study
wherein
A: 0.05-0.5
B: 0.5-5
C: >5
wherein
A: 0.03-0.1
B: 0.1-0-3
C: 0.3-1
wherein
A: 50-150
B: 10-50
C: 1-10
wherein
A: 10-20
B: 1-10
C: 0.1-1

Example B9: In Vivo Xenograft Cancer Mouse Model Study

The anti-tumor activities of test compounds are examined in murine xenograft models of human cancer. For this purpose, immunocompromised mice are implanted subcutaneously with tumor cells or tumor fragments. At a mean tumor size of 20-40 mm², animals are randomized split into treatment and control groups (n=10 animals/group) and treatment starts with vehicle-only, or test compound (formulation: phosphate buffered saline (“PBS”); application route: intravenously into the tail vein (“i.v.”)). Intravenous treatments are performed on two consecutive days once daily followed by five days of drug holiday without any treatment. The tumor size and the body weight are determined twice weekly. The tumor area is detected by means of an electronic caliper [length (mm)×width (mm)]. The experimental groups are ended when the groups reached the pre-determined ethical endpoint based on German and European animal welfare regulations. In vivo anti-tumor efficacy is presented as T/C ratio of mean tumor area measured for treatment and control group on the last day at which the vehicle control remains in study (Treatment/Control; mean tumor area of treatment group/mean tumor area of control group. A compound having a T/C below 0.5 is defined as active (i.e., effective). Statistical analysis is assessed using SigmaStat software. A one-way analysis of variance is performed and differences to the control are compared by a pair-wise comparison procedure (Dunn's method).

1. A compound, or a pharmaceutically acceptable salt thereof, or a stereoisomer or mixture of stereoisomers thereof, comprising a PTEFb inhibitor conjugated to one or more integrin binders via a linker.

2. The compound of claim 1, or a pharmaceutically acceptable salt thereof, or a stereoisomer or mixture of stereoisomers thereof, having a structure of Formula (V):
IN-L-EL-PT Formula (V)
wherein:
IN is an integrin binder;
L is a linker, optionally further comprising a second integrin binder;
EL is a peptide linker, optionally further comprising a self-immolative group; and
PT is a PTEFb inhibitor.

3. The compound of claim 1 or 2, or a pharmaceutically acceptable salt thereof, or a stereoisomer or mixture of stereoisomers thereof, wherein EL is enzymatically cleavable.

4. The compound of any one of claims 1-3, or a pharmaceutically acceptable salt thereof, or a stereoisomer or mixture of stereoisomers thereof, having a structure of Formula (V-0):
IN-L-AA¹-AA²-(AA³)₀₋₁-(SIL)₀₋₁-PT Formula (V-0)
wherein:
PT is a PTEFb inhibitor;
SIL is a self-immolative linker;
AA¹ is an amino acid;
AA² is an amino acid;
AA³ is an amino acid;
L is a linker, optionally further comprising a second integrin binder; and
IN is an integrin binder.

5. The compound of any one of claims 1-4, or a pharmaceutically acceptable salt thereof, or a stereoisomer or mixture of stereoisomers thereof, having a structure of Formula (V-1), Formula (V-2), Formula (V-3), or Formula (V-4):
IN-L-AA¹-AA²-AA³SIL-PT Formula (V-1)
IN-L-AA¹-AA²-AA³-PT Formula (V-2)
IN-L-AA¹-AA²-SIL-PT Formula (V-3)
IN-L-AA¹-AA²-PT Formula (V-4)
wherein:
PT is a PTEFb inhibitor;
SIL is a self-immolative linker;
AA¹ is an amino acid;
AA² is an amino acid;
AA³ is an amino acid;
L is a linker, optionally further comprising a second integrin binder; and
IN is an integrin binder.

6. The compound of any one of claims 1-5, or a pharmaceutically acceptable salt thereof, or a stereoisomer or mixture of stereoisomers thereof, having a structure of Formula (V-A2), Formula (V-A3), Formula (V-C₂), or Formula (V-C₃): 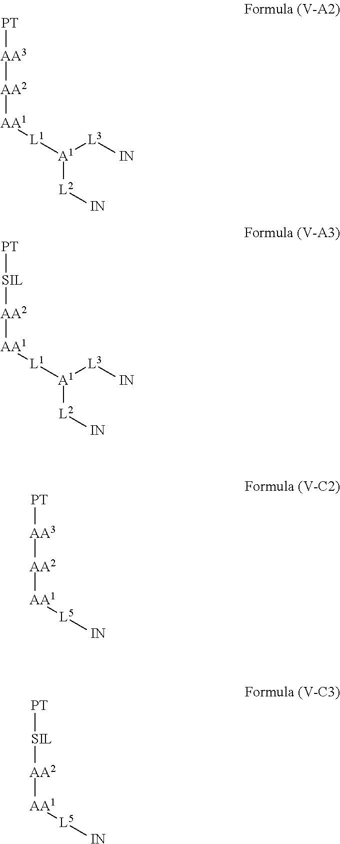
wherein:
PT is a PTEFb inhibitor;
SIL is a self-immolative linker (e.g., a PABC linker);
each AA¹, AA², and AA³ is independently an amino acid,
each L¹, L², L³, and L⁵ is independently a bivalent linker,
A¹ is a trivalent linker; and
IN is an integrin binder.

7. The compound of any one of claims 1-6, or a pharmaceutically acceptable salt thereof, or a stereoisomer or mixture of stereoisomers thereof, wherein IN is a peptidic or peptidomimetic integrin binder.

8. The compound of any one of claims 1-7, or a pharmaceutically acceptable salt thereof, or a stereoisomer or mixture of stereoisomers thereof, wherein IN is small molecule integrin binder.

9. The compound of any one of claims 1-8, or a pharmaceutically acceptable salt thereof, or a stereoisomer or mixture of stereoisomers thereof, wherein IN is a small molecule αᵥβ₃ integrin binder.

10. The compound of any one of claims 1-9, or a pharmaceutically acceptable salt thereof, or a stereoisomer or mixture of stereoisomers thereof, having a structure of Formula (VI-A) or Formula (VI-C): 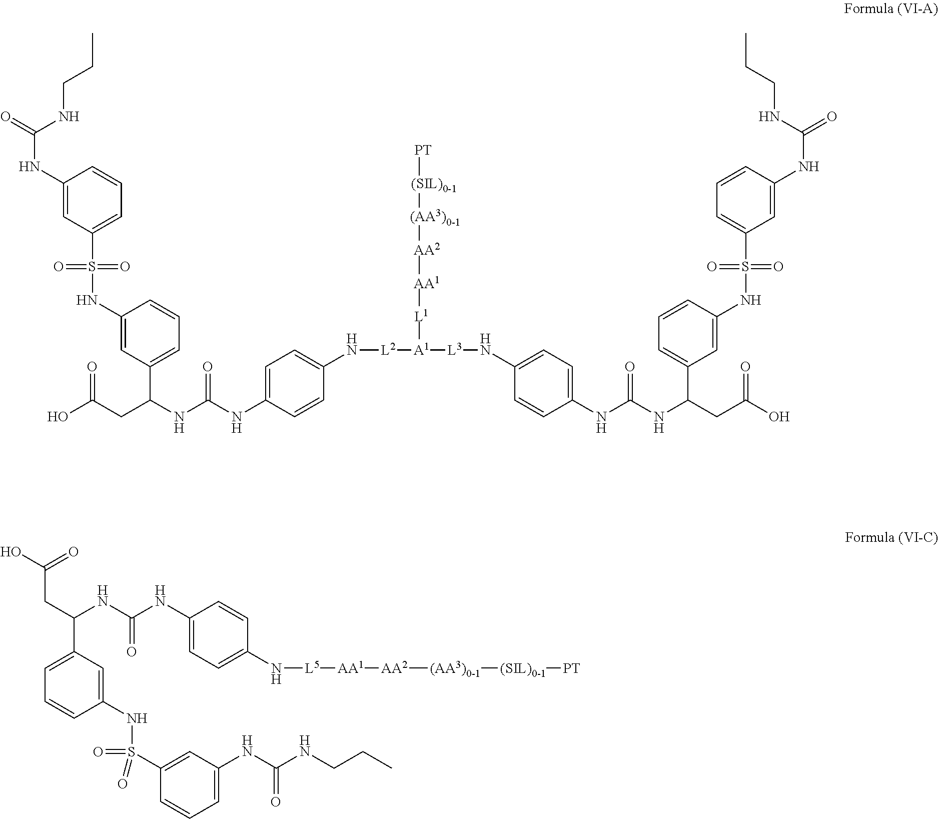
wherein:
PT is a PTEFb inhibitor;
each AA¹, AA², and AA³ is independently an amino acid,
SIL is a self-immolative linker (e.g., a PABC linker);
L¹, L², L³, and L⁵ are bivalent linkers,
A¹ is a trivalent linker; and
IN is an integrin binder.

11. The compound of any one of claims 1-10, or a pharmaceutically acceptable salt thereof, wherein EL is cleaved by cathepsin B, legumain, or neutrophil elastase.

12. The compound of any one of claims 1-11, or a pharmaceutically acceptable salt thereof, wherein SIL is absent, and EL is cleaved by neutrophil elastase.

13. The compound of any one of claims 1-12, or a pharmaceutically acceptable salt thereof, wherein EL has the formula: L-Asp-L-Pro-L-Val, L-Asn-L-Pro-L-Val, -Gly-L-Pro-L-Val-, L-Ala-L-Pro-L-Val-, L-Nva-L-Pro-L-Val-, L-His-L-Pro-L-Val-, L-Asp-L-Pro-L-Ala, L-Asn-L-Pro-L-Ala, L-Asp-L-Pro-L-Ile, L-Asn-L-Pro-L-Ile, -Gly-L-Pro-L-Ile-, L-Ala-L-Pro-L-Ile-, L-Nva-L-Pro-L-Ile-, L-His-L-Pro-L-Ile-, L-Asp-L-Pro-L-Leu, L-Asn-L-Pro-L-Leu, -Gly-L-Pro-L-Leu-, L-Ala-L-Pro-L-Leu-, L-Nva-L-Pro-L-Leu-, or L-His-L-Pro-L-Leu-.

14. The compound of any one of claims 1-11, or a pharmaceutically acceptable salt thereof, wherein SIL is absent, and EL is cleaved by legumain.

15. The compound of any one of claims 1-11 or 14, or a pharmaceutically acceptable salt thereof, wherein EL has the formula: -L-Ala-L-Ala-L-Asp-, -L-Ala-L-Ala-L-Asn-, -L-Ala-L-Asp-L-Asn, -L-Ala-L-Asn-, -L-Asp-L-Asn-, -L-Ala-N-Me L-Ala-L-Asn-, -L-Ala-D-His-L-Asn-, -L-Ala-D-Asp-L-Asn-, -L-Ala-D-Ala-L-Asn-, or -L-Ala-D-Ser-L-Asn-.

16. The compound of any one of claims 1-11, or a pharmaceutically acceptable salt thereof, wherein EL is cleaved by cathepsin.

17. The compound of any one of claims 1-11 or 16, or a pharmaceutically acceptable salt thereof, wherein EL has the formula: -L-Val-L-Cit-, L-Phe-L-Cit-, -L-Leu-L-Cit-, -L-Val-L-Ala-, -L-Phe-L-Lys-, -L-Ala-L-Lys-, or -L-Val-L-Lys-.

18. The compound of any one of claims 1-13, or a pharmaceutically acceptable salt thereof; or a stereoisomer or mixture of stereoisomers thereof, having a structure of Formula (II-A) or Formula (II-C): 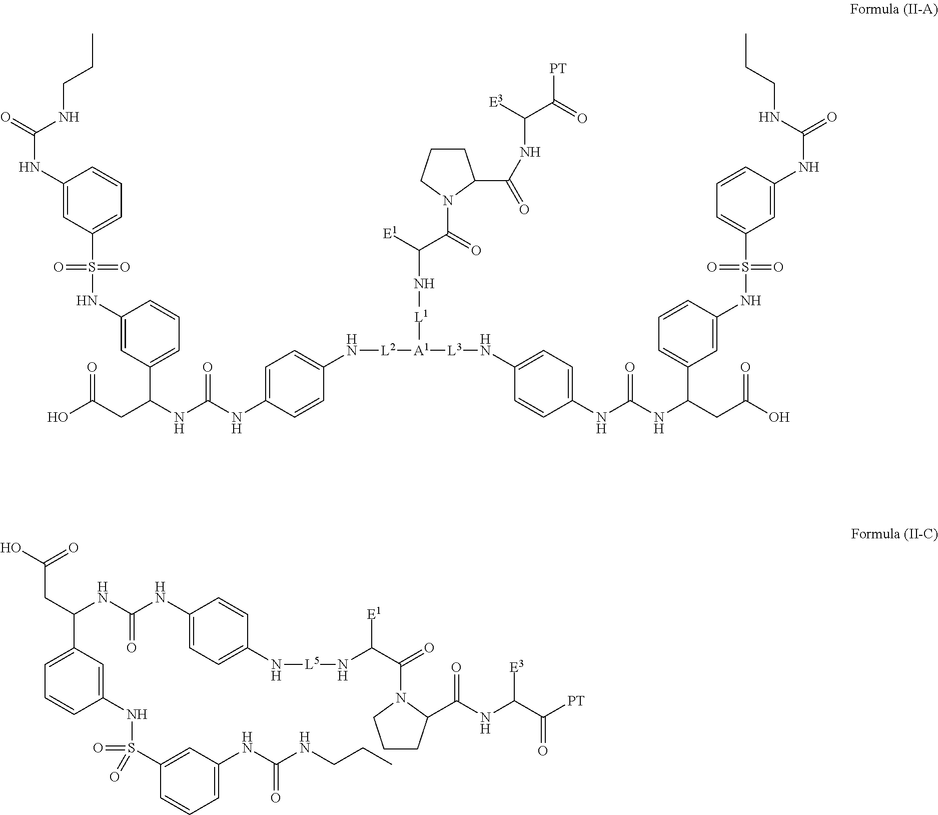
wherein:
PT is a PTEFb inhibitor;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
E³ is —CH₃, —CH(CH₃)₂, —CH₂CH(CH₃)₂, or —CH(CH₃)CH₂CH₃;
A¹ is a trivalent linker;
each of L¹, L², L³, and L⁵ is independently a bivalent linker;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
wherein:
L⁶ is a bivalent linker; and
IN is an integrin binder.

19. The compound of any one of claims 1-11, or 11, or a pharmaceutically acceptable salt thereof, or a stereoisomer or mixture of stereoisomers thereof, having a structure represented by Formula (XI-C): 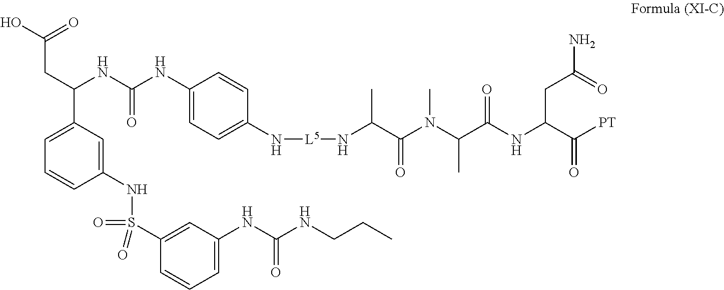
wherein:
PT is a PTEFb inhibitor; and
L⁵ is a bivalent linker.

20. The compound of any one of claims 1-11, or 16-17, or a pharmaceutically acceptable salt thereof, or a stereoisomer or mixture of stereoisomers thereof, having a structure represented by Formula (VI-C2): 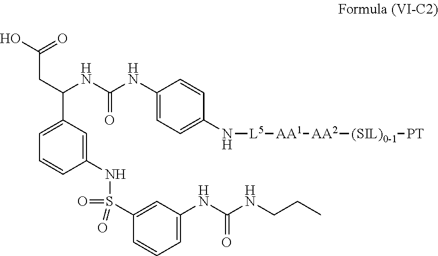
wherein:
PT is a PTEFb inhibitor;
SIL is a self-immolative linker;
AA¹ is Ala, Leu, Phe, or Val;
AA² is Ala, Cit, or Lys; and
L⁵ is a bivalent linker.

21. The compound of any one of claims 1-20, or a pharmaceutically acceptable salt thereof; or a stereoisomer or mixture of stereoisomers thereof, wherein PT has a structure of the formula: 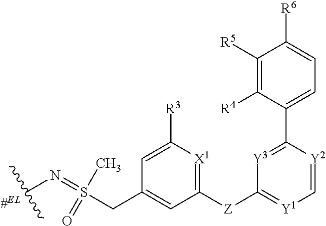
or a pharmaceutically acceptable salt thereof; or a stereoisomer or mixture of stereoisomers thereof, wherein:
#^{EL} denotes a bond to EL (or SIL);
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl; or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl; or R³ and R⁵ are taken together to form a bivalent radical of the formula —O—C₂₋₁₀ alkyl-O—, —NH—C₂₋₁₀ alkyl-O—, or —NH—C₂₋₁₀ alkyl-NH—; and
R⁶ is hydrogen, halogen, —OH, or —O—C₁₋₄alkyl.

22. The compound of any one of claims 1-13, 18, or 21, or a pharmaceutically acceptable salt thereof; or a stereoisomer or mixture of stereoisomers thereof, having a structure of Formula (III-A) or Formula (III-C): 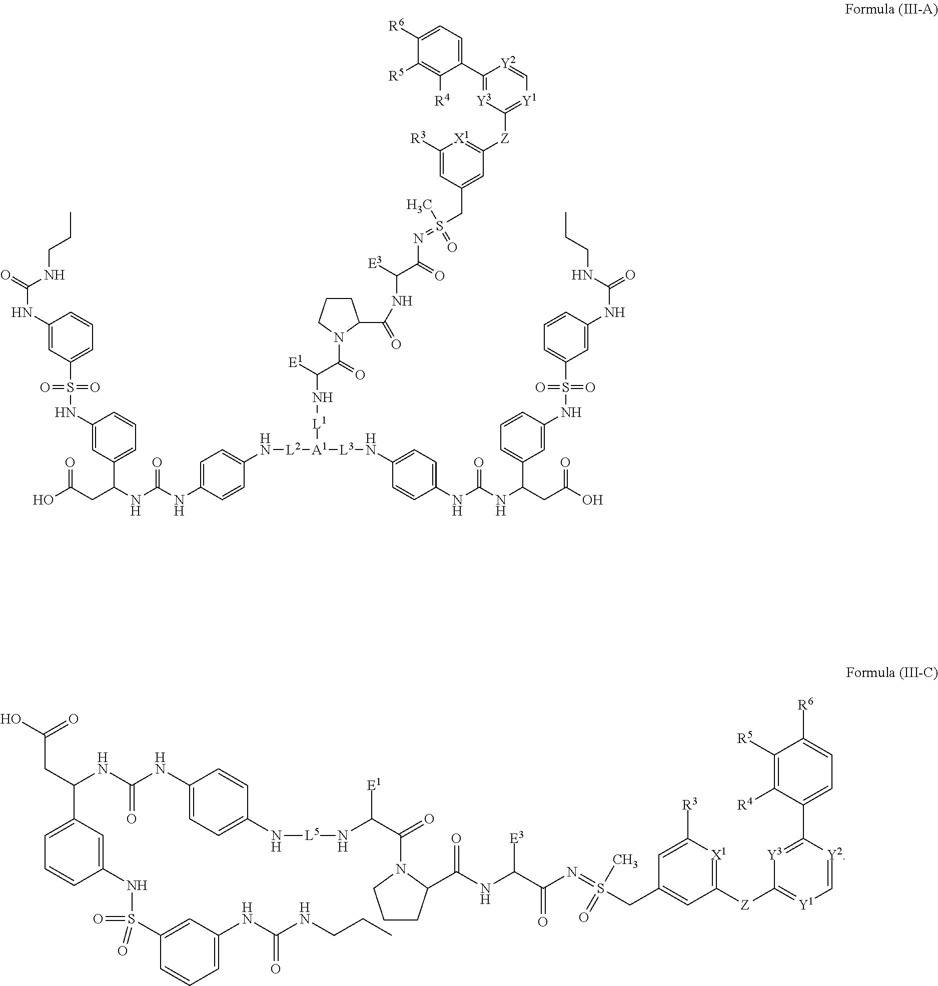
wherein:
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH, or —CH₂CH₂C(O)OR¹;
R¹ is C₁₋₆alkyl substituted with —NH₂, —N(CH₃)₂, —N(CH₃)₃⁺, or -NHL⁶-IN;
IN is an integrin binder;
E³ is —CH₃, —CH(CH₃)₂, —CH₂CH(CH₃)₂, or —CH(CH₃)CH₂CH₃;
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl; or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl; or R³ and R⁵ are taken together to form a bivalent radical of the formula —O—C₂₋₁₀ alkyl-O—, —O—C₂₋₁₀ alkyl-NH—, or —NH—C₂₋₁₀ alkyl-NH—;
R⁶ is hydrogen, halogen, —OH, or —O—C₁₋₄alkyl;
A¹ is a trivalent linker; and
each of L¹, L², L³, and L⁵ is independently a bivalent linker;

23. The compound of any one of claims 1-11, 14-15, 19, or 21, or a pharmaceutically acceptable salt thereof, or a stereoisomer or mixture of stereoisomers thereof, having a structure represented by Formula (XII-C): 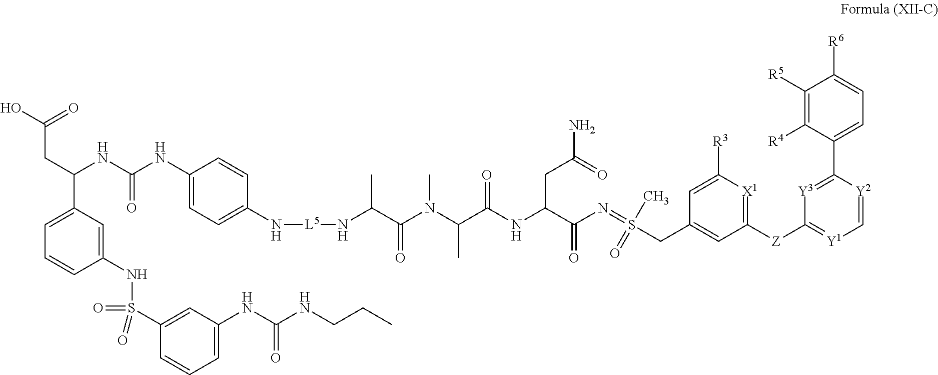
wherein:
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl; or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl; or R³ and R⁵ are taken together to form a bivalent radical of the formula —O—C₂₋₁₀ alkyl-O—, —O—C₂₋₁₀ alkyl-NH—, or —NH—C₂₋₁₀ alkyl-NH—;
R⁶ is hydrogen, halogen, —OH, or —O—C₁₋₄alkyl; and
L⁵ is a bivalent linker.

24. The compound of any one of claims 1-11, 16-17, or 20-21, or a pharmaceutically acceptable salt thereof, or a stereoisomer or mixture of stereoisomers thereof, having a structure represented by Formula (VII-C): 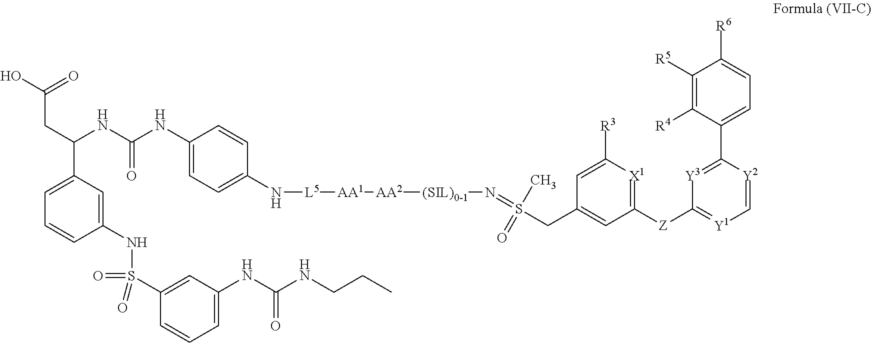
wherein:
X¹ is CH, CF, or N;
Y¹ is CH, CF, or N;
Y² is CH, CF, or N;
Y³ is CH, CF, or N;
Z is —CH₂—, —C(═O)NH— —NH—, —N(CH₃)—, —NHC(═O)—, —O—, or —S—;
R³ is hydrogen, halogen, —OH, or —O—C₁₋₄alkyl;
R⁴ is hydrogen, halogen, —OH, or —O—C₁₋₄alkyl;
or R³ and R⁴ are taken together to form a bivalent radical of the formula —O—C₂₋₁₀ alkyl-O—, —NH—C₂₋₁₀ alkyl-O—, or —NH—C₂₋₁₀ alkyl-NH—;
R⁵ is hydrogen, halogen, —OH, or —O—C₁₋₄alkyl;
or R³ and R⁵ are taken together to form a bivalent radical of the formula —O—C₂₋₁₀ alkyl-O—, —O—C₂₋₁₀ alkyl-NH—, or —NH—C₂₋₁₀ alkyl-NH—;
R⁶ is hydrogen, halogen, —OH, or —O—C₁₋₄alkyl;
AA¹ is Ala or Val;
AA² is Ala, Cit, or Lys;
SIL is a self-immolative linker; and
L⁵ is a linker (e.g., a simple spacer or a polymeric spacer, defined herein).

25. The compound of any one of the preceding claims, or a pharmaceutically acceptable salt thereof; or a stereoisomer or mixture of stereoisomers thereof, wherein each of L¹, L², L³, and L⁵ is a bivalent linker having a structure represented by formula (i), (ii), or (iii) below:
—(CO)ₘ(CH₂)ₙ(OC₂₋₆ alkyl)ₒ(NH)ₚ(CO)_{q}—; (i)
—(CO)ᵣ(CH₂)ₛ(NR¹⁰C₁₋₆ alkyl)ₜ(NR¹¹)ᵤ(CO)ᵥ—; or (ii)
—(CO)ᵣ(CH₂)ₛ(NR¹⁰C(O)C₁₋₆ alkyl)ₜ(NR¹¹)ᵤ(CO)ᵥ—; (iii)
wherein:
R¹⁰ is, in each instance, independently selected from hydrogen or C₁₋₃ alkyl;
R¹¹ is, in each instance, independently selected from hydrogen or C₁₋₃ alkyl;
m is 0 or 1; n is 0 to 10; o is 1 to 10; p is 0 or 1; and q is 0 or 1; and
r is 0 or 1; s is 0 to 10; t is 1 to 10; u is 0 or 1; and v is 0 or 1.

26. The compound of any one of the preceding claims, or a pharmaceutically acceptable salt thereof; or a stereoisomer or mixture of stereoisomers thereof, wherein:
L¹ is —C(O)—C₂₋₄ alkyl-[O—C₂₋₆ alkyl]₁₋₈-NH—; or
—C(O)—C₁₋₄ alkyl-[N(CH₃)—C₁₋₆ alkyl]₁₋₈-NH—,
L² is —C(O)—C₂₋₄ alkyl-[O—C₂₋₆ alkyl]₁₋₈-NHC(O)—; or
—C(O)—C₁₋₄ alkyl-[N(CH₃)—C₁₋₆ alkyl]₁₋₈-NHC(O)—,
L³ is —C(O)—C₂₋₄ alkyl-[O—C₂₋₆ alkyl]₁₋₈-NHC(O)—; or
—C(O)—C₁₋₄ alkyl-[N(CH₃)—C₁₋₆ alkyl]₁₋₈-NHC(O)—; and
L⁵ is —C(O)—C₂₋₄ alkyl-[O—C₂₋₆ alkyl]₁₋₈-NHC(O)—;
—C(O)—C₁₋₄ alkyl-[N(CH₃)—C₁₋₆ alkyl]₁₋₈-NHC(O)—;
—C(O)—C₁₋₄ alkyl-[N(CH₃)—C₁₋₆ alkyl]₁₋₈-N(CH₃)C(O)—;
—C(O)—C₁₋₄ alkyl-[N(CH₃)C(O)—C₁₋₆ alkyl]₁₋₈-NHC(O)—; or
—C(O)—C₁₋₄ alkyl-[N(CH₃)C(O)—C₁₋₆ alkyl]₁₋₈-N(CH₃)C(O)—.

27. The compound of any one of the preceding claims, or a pharmaceutically acceptable salt thereof; or a stereoisomer or mixture of stereoisomers thereof, wherein:
L¹ is —C(O)—C₂₋₄ alkyl-[O—C₂₋₄ alkyl]₂₋₄-NH—*;
or —C(O)—C₁₋₄ alkyl-[N(CH₃)—C₁₋₄ alkyl]₂₋₄-NH—*,
L² is **—C(O)—C₂₋₄ alkyl-[O—C₂₋₄ alkyl]₂₋₄-NHC(O)—;
or **—C(O)—C₁₋₄ alkyl-[N(CH₃)—C₂₋₄ alkyl]₂₋₄-NHC(O)—,
L³ is ***—C(O)—C₂₋₄ alkyl-[O—C₂₋₄ alkyl]₂₋₄-NHC(O)—;
or ***—C(O)—C₁₋₄ alkyl-[N(CH₃)—C₂₋₄ alkyl]₂₋₄-NHC(O)—;
A¹ is a trivalent linker of the formula: 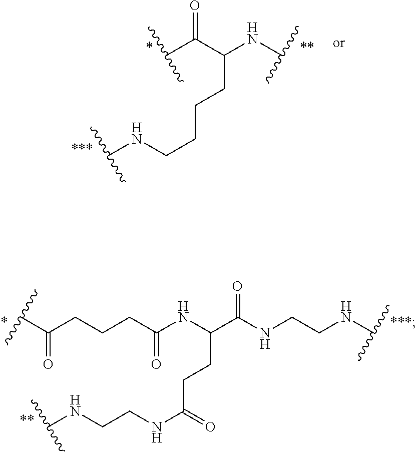 [SMILES: *NCCCCC(N*)C(*)=O ; *NCCNC(=O)CCC(NC(=O)CCCC(*)=O)C(=O)NCCN*]
wherein:
* denotes a bond from L¹ to A¹;
** denotes a bond from A¹ to L²; and
*** denotes a bond from A¹ to L³.

28. The compound of any one of the preceding claims, or a pharmaceutically acceptable salt thereof; or a stereoisomer or mixture of stereoisomers thereof, wherein:
L⁵ is —C(O)—C₂₋₄ alkyl-[O—C₂₋₄ alkyl]₂₋₄-NHC(O)—^{#};
—C(O)—C₁₋₄ alkyl-[N(CH₃)—C₁₋₄ alkyl]₂₋₄-NHC(O)—^{#};
—C(O)—C₁₋₄ alkyl-[N(CH₃)—C₁₋₄ alkyl]₂₋₄-N(CH₃)C(O)—^{#};
—C(O)—C₁₋₄ alkyl-[N(CH₃)C(O)—C₁₋₄ alkyl]₂₋₄-NHC(O)—^{#}; or
—C(O)—C₁₋₄ alkyl-[N(CH₃)C(O)—C₁₋₄ alkyl]₂₋₄-N(CH₃)C(O)—^{#};
wherein ^{#} denotes a bond from L⁵ to the integrin binder.

29. The compound of any one of the preceding claims, or a pharmaceutically acceptable salt thereof; or a stereoisomer or mixture of stereoisomers thereof, wherein PT has a structure: 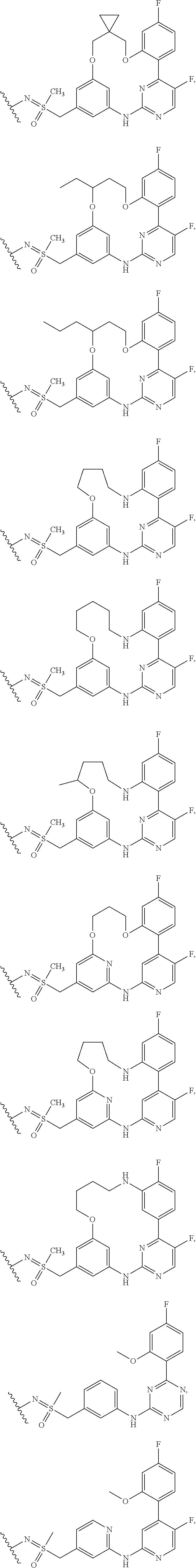 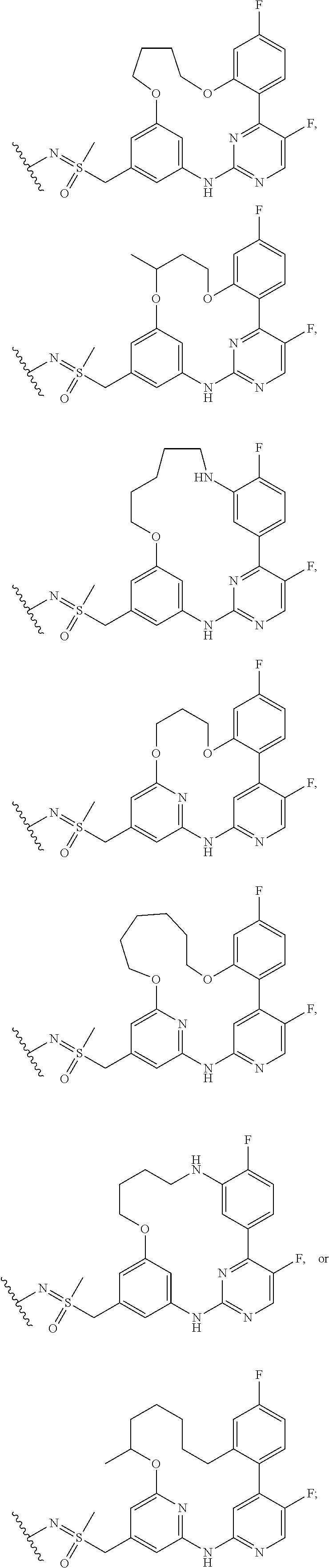
or a pharmaceutically acceptable salt thereof, or a stereoisomer or mixture of stereoisomers thereof.

30. The compound of any one of the preceding claims, or a pharmaceutically acceptable salt thereof, or a stereoisomer or mixture of stereoisomers thereof, wherein PT has a structure: 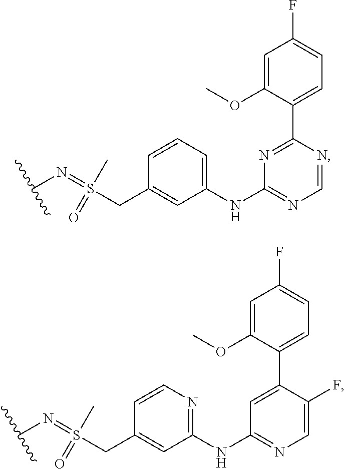 [SMILES: *N=S(C)(=O)Cc1cccc(Nc2ncnc(-c3ccc(F)cc3OC)n2)c1 ; *N=S(C)(=O)Cc1ccnc(Nc2cc(-c3ccc(F)cc3OC)c(F)cn2)c1] 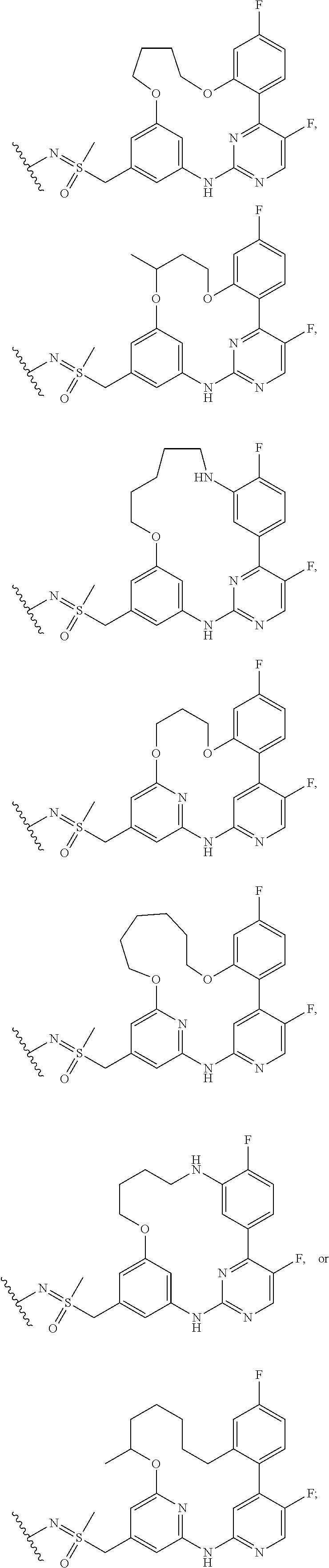
or a pharmaceutically acceptable salt thereof; or a stereoisomer or mixture of stereoisomers thereof.

31. The compound of any one of the preceding claims, or a pharmaceutically acceptable salt thereof; or a stereoisomer or mixture of stereoisomers thereof, wherein:
E¹ is —CH₂C(O)OH or —CH₂C(O)OR¹;
R¹ is C₁₋₆ alkyl substituted with —NH₂, —N(CH₃)₂, or —N(CH₃)₃⁺;
E³ is —CH₃ or —CH(CH₃)₂;
L¹ is —C(O)—C₂₋₄ alkyl-[O—C₂₋₄ alkyl]₂₋₄-NH—*;
L² is **—C(O)—C₂₋₄ alkyl-[O—C₂₋₄ alkyl]₂₋₄-NHC(O)—;
L³ is ***—C(O)—C₂₋₄ alkyl-[O—C₂₋₄ alkyl]₂₋₄-NHC(O)—;
L⁵ is —C(O)—C₂₋₄ alkyl-[O—C₂₋₄ alkyl]₂₋₄-NHC(O)—^{#};
A¹ is a trivalent linker of the formula: 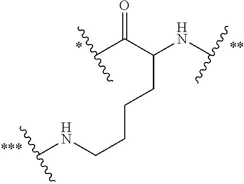 [SMILES: *NCCCCC(N*)C(*)=O]
wherein:
** denotes a bond from L¹ to A¹;
** denotes a bond from A¹ to L²;
*** denotes a bond from A¹ to L³; and
^{#} denotes a bond from L⁵ to the integrin binder.

32. The compound of any one of the preceding claims, or a pharmaceutically acceptable salt thereof, or a stereoisomer or mixture of stereoisomers thereof, having a structure represented by Formula (III-A3), Formula (III-C3), or Formula (III-C4): 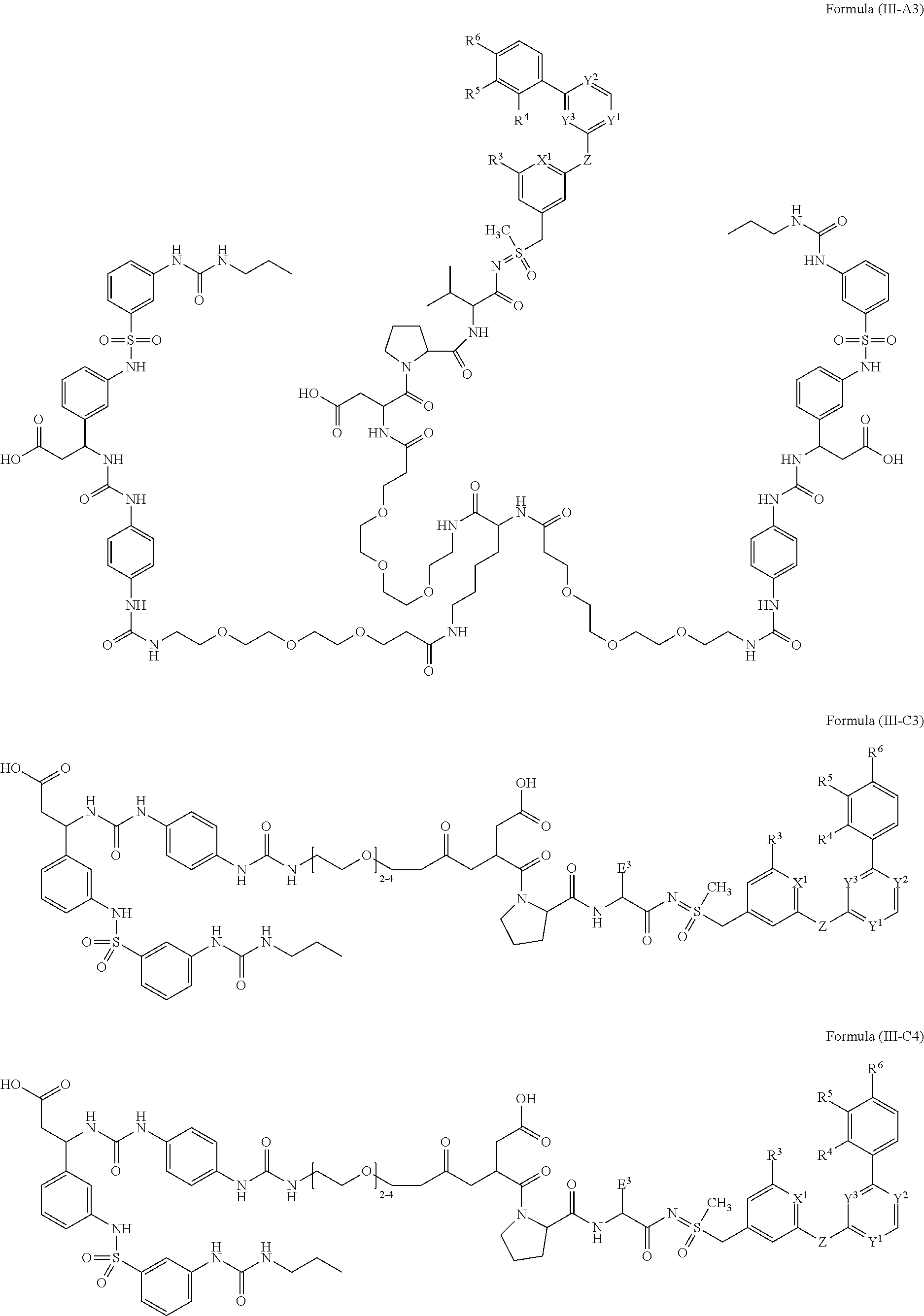
wherein:
R¹ is —C₁₋₆ alkyl-NH₂, —C₁₋₆ alkyl-N(CH₃)₂, or —C₁₋₆ alkyl-N(CH₃)₃⁺; and
E³ is —CH₃ or —CH(CH₃)₂.

33. The compound of any one of the preceding claims, or a pharmaceutically acceptable salt thereof; or a stereoisomer or mixture of stereoisomers thereof, having the structure: 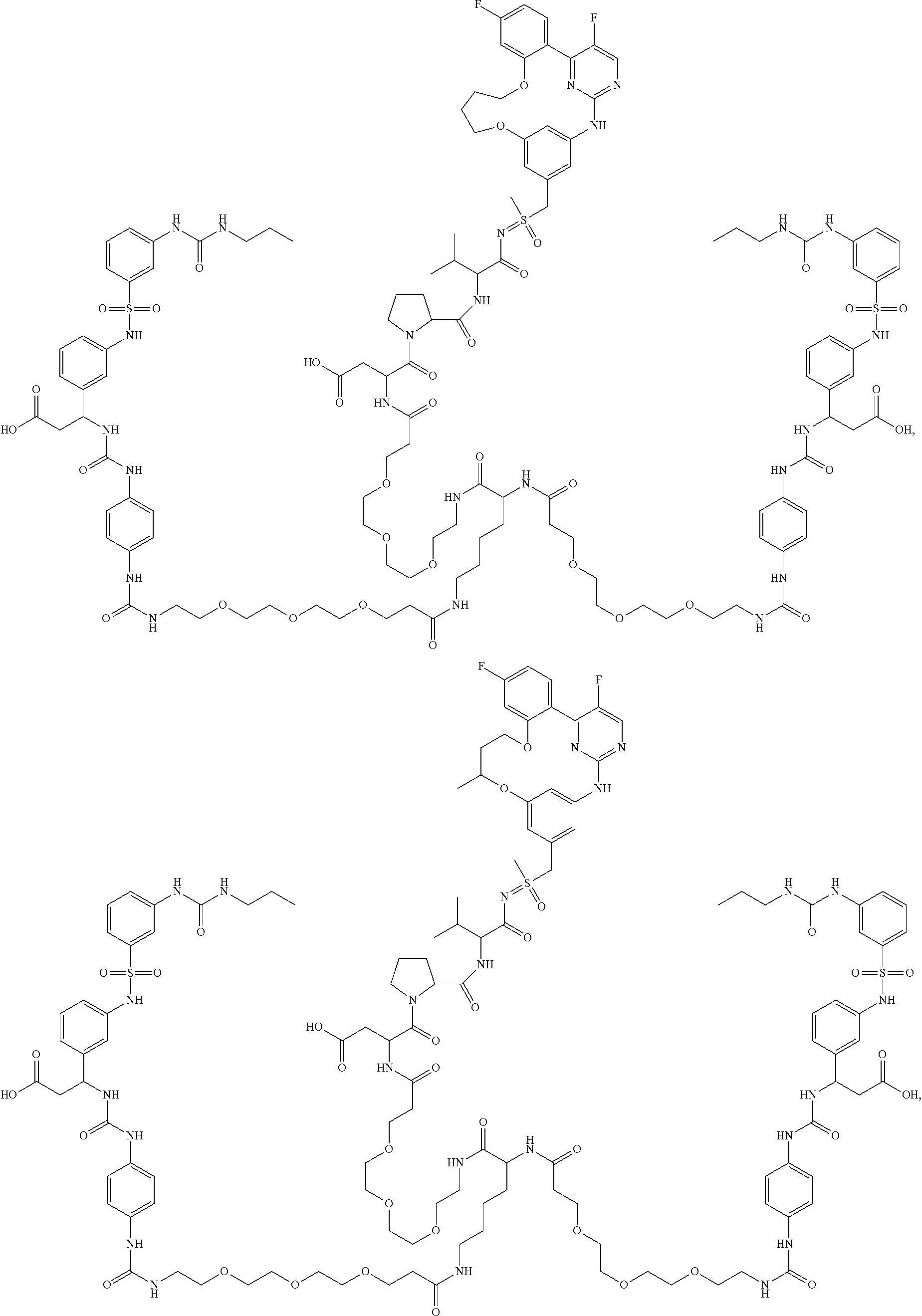 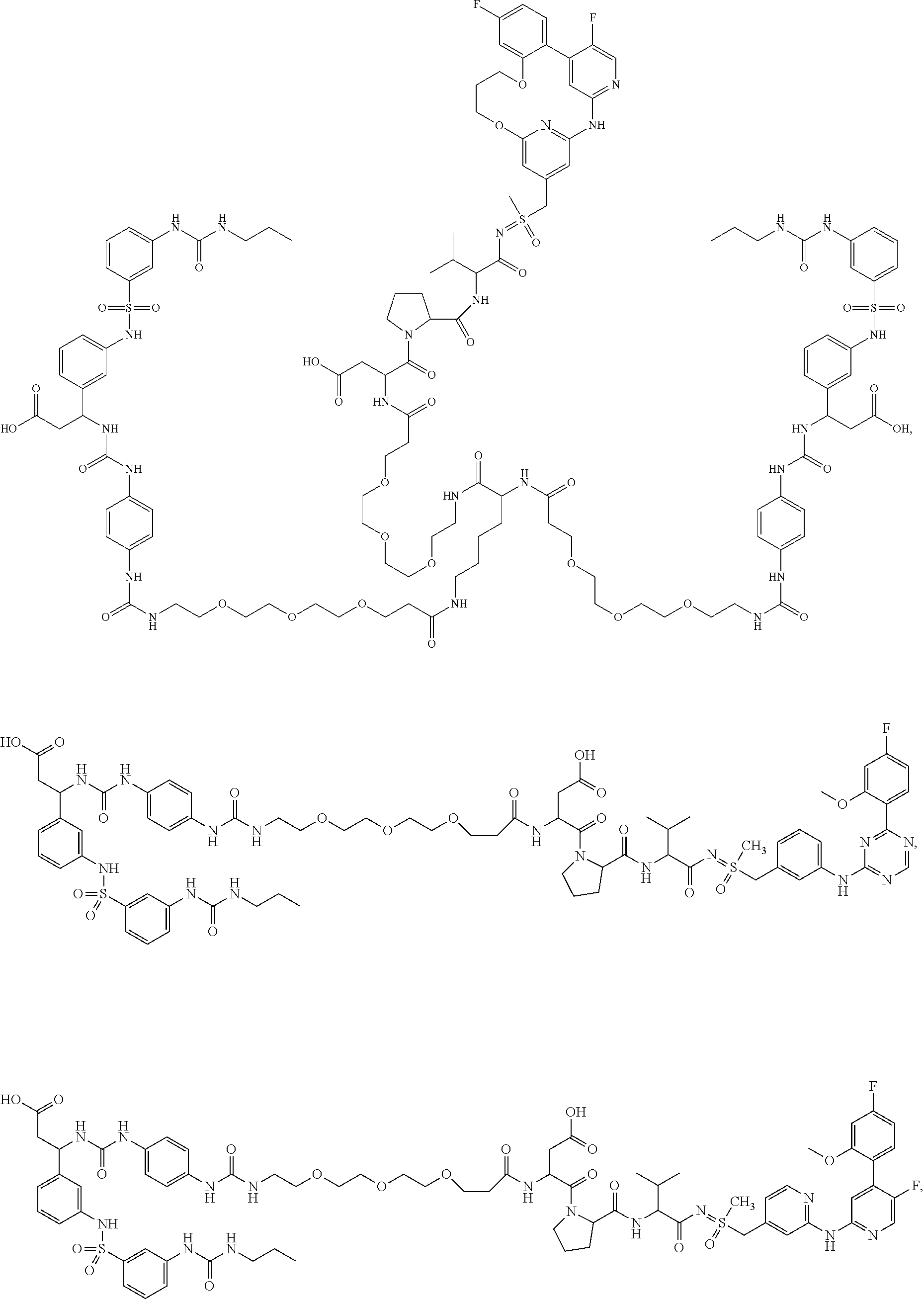 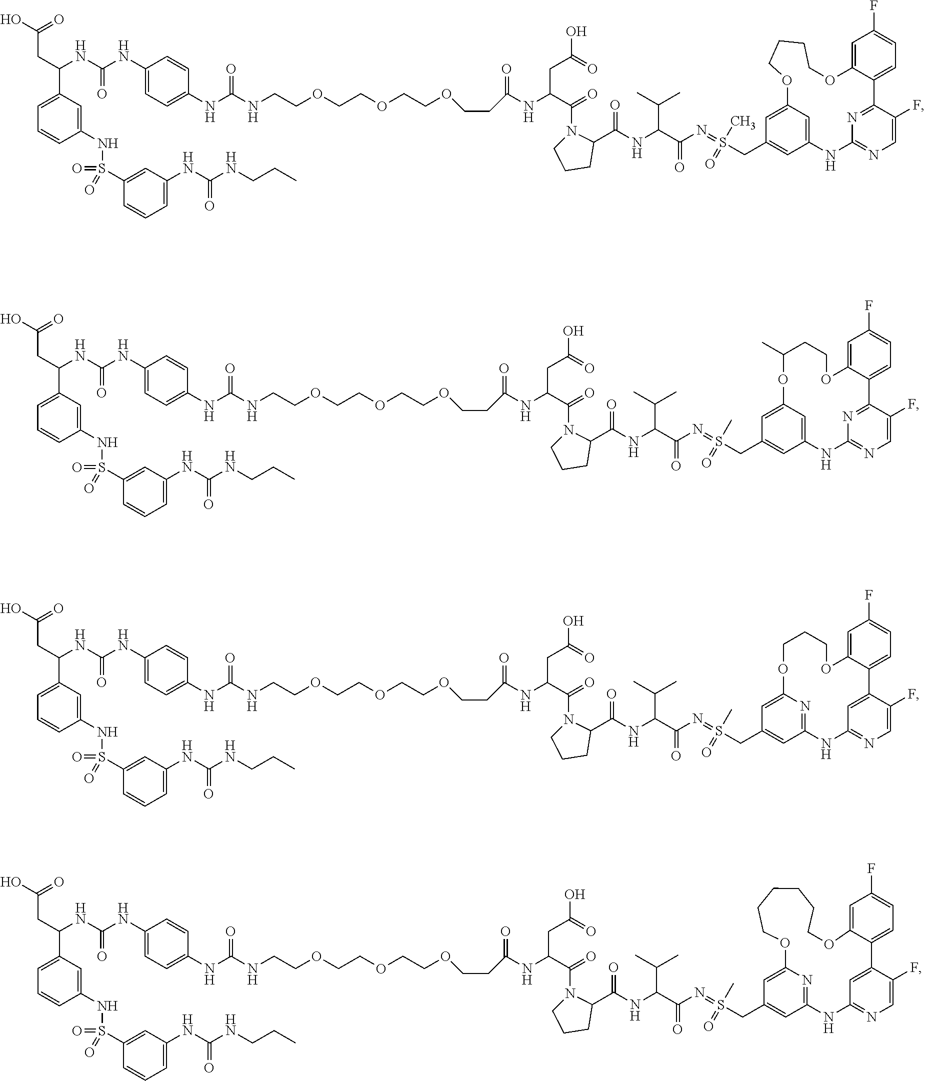 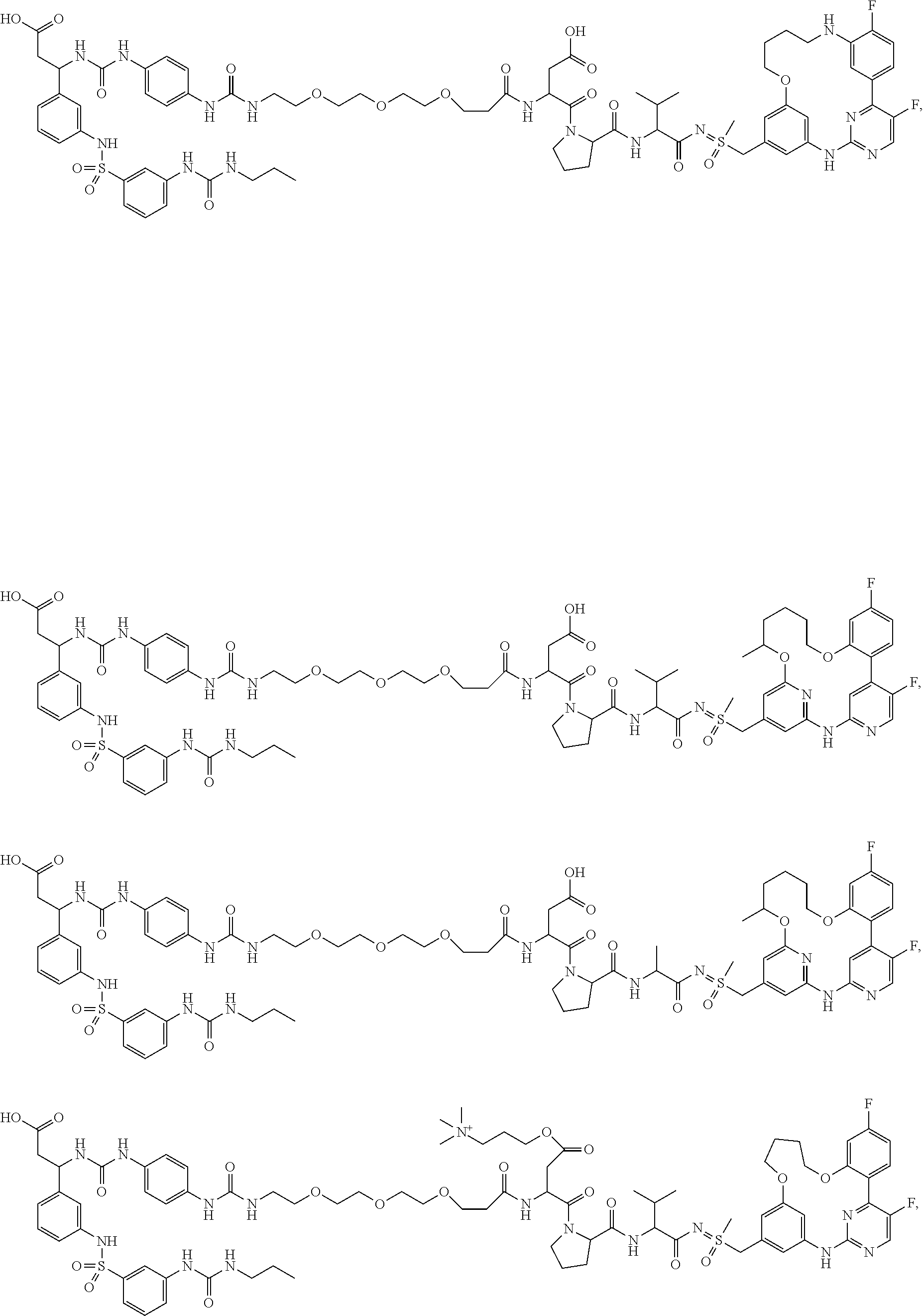 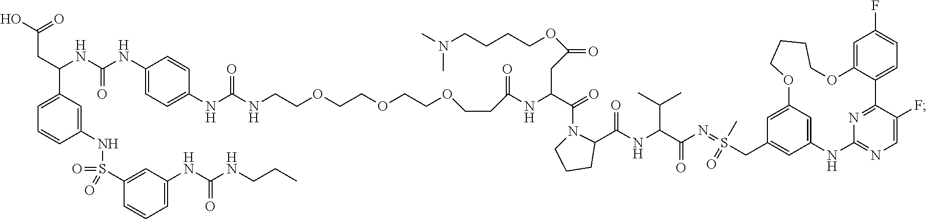 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)OCCCCN(C)C)C(=O)N4CCCC4C(=O)NC(C(=O)N=S(C)(=O)Cc4cc5cc(c4)OCCCCOc4cc(F)ccc4-c4nc(ncc4F)N5)C(C)C)cc3)c2)c1]
or a pharmaceutically acceptable salt thereof; or a stereoisomer or mixture of stereoisomers thereof.

34. The compound of any one of the preceding claims, or a pharmaceutically acceptable salt thereof; or a stereoisomer or mixture of stereoisomers thereof, wherein:
E¹ is hydrogen or —CH₂C(O)NH₂;
E³ is —CH(CH₃)₂; and
L¹ is —C(O)—C₁₋₄ alkyl-[N(CH₃)—C₁₋₄ alkyl]₂₋₄-NHC(O)—*;
L² is **—C(O)—C₁₋₄ alkyl-[N(CH₃)—C₁₋₄ alkyl]₂₋₄-NHC(O)—;
L³ is ***—C(O)—C₁₋₄ alkyl-[N(CH₃)—C₁₋₄ alkyl]₂₋₄-NHC(O)—;
L⁵ is —C(O)—C₁₋₄ alkyl-[N(CH₃)—C₁₋₄ alkyl]₂₋₄-NHC(O)—^{#};
or —C(O)—C₁₋₄ alkyl-[N(CH₃)—C₁₋₄ alkyl]₂₋₄-N(CH₃)C(O)—^{#};
A¹ is a trivalent linker of the formula: 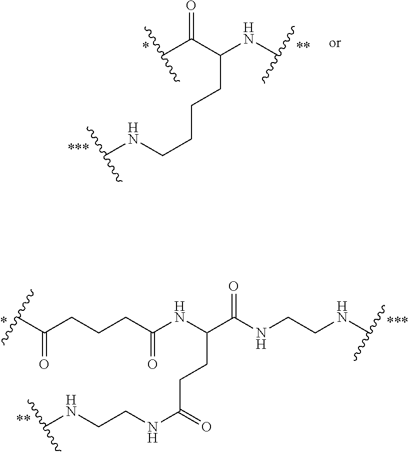 [SMILES: *NCCCCC(N*)C(*)=O ; *NCCNC(=O)CCC(NC(=O)CCCC(*)=O)C(=O)NCCN*]
wherein:
** denotes a bond from L¹ to A¹;
** denotes a bond from A¹ to L²;
*** denotes a bond from A¹ to L³; and
^{#} denotes a bond from L⁵ to the integrin binder.

35. The compound of any one of the preceding claims, or a pharmaceutically acceptable salt thereof, or a stereoisomer or mixture of stereoisomers thereof, having a structure represented by Formula (III-A4), Formula (III-A5), Formula (III-C4), or Formula (III-C5):

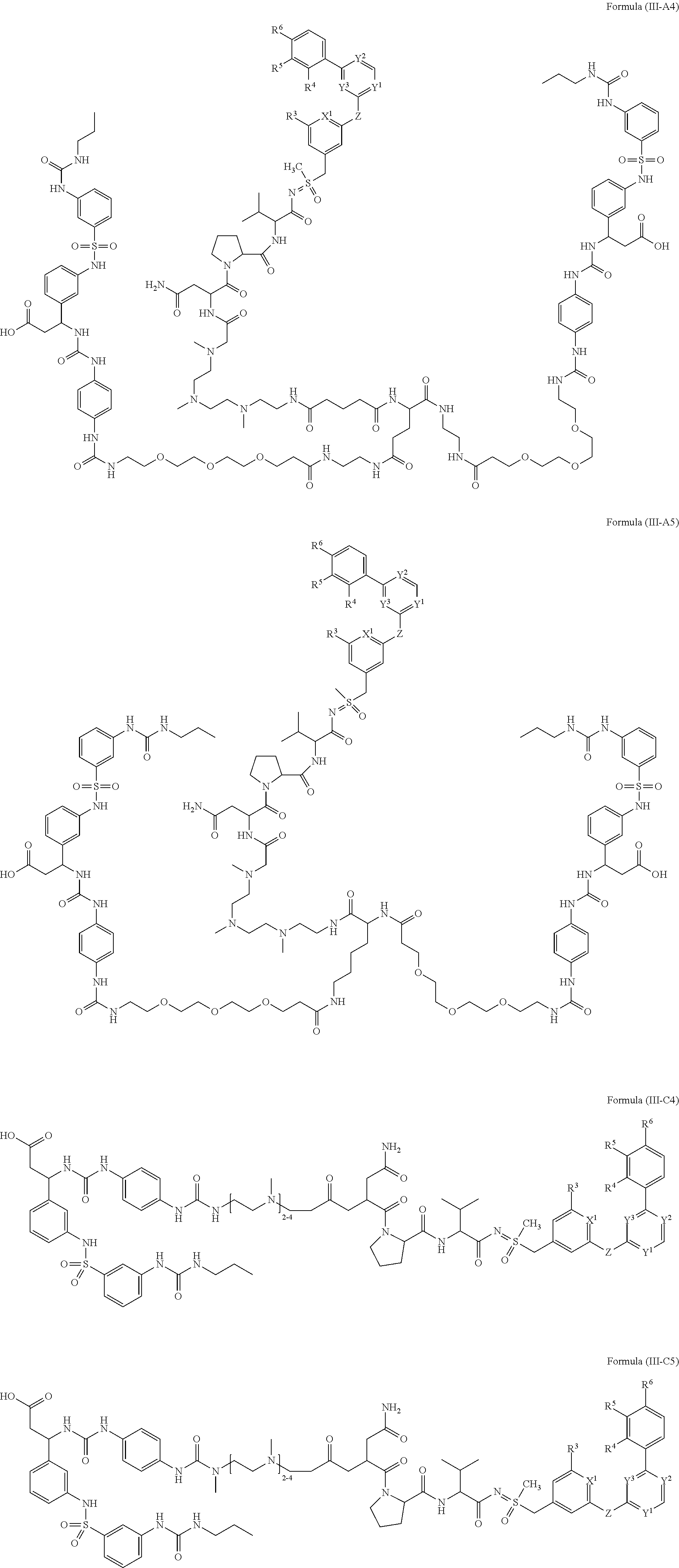

36. The compound of any one of the preceding claims, or a pharmaceutically acceptable salt thereof, or a stereoisomer or mixture of stereoisomers thereof, having the structure: 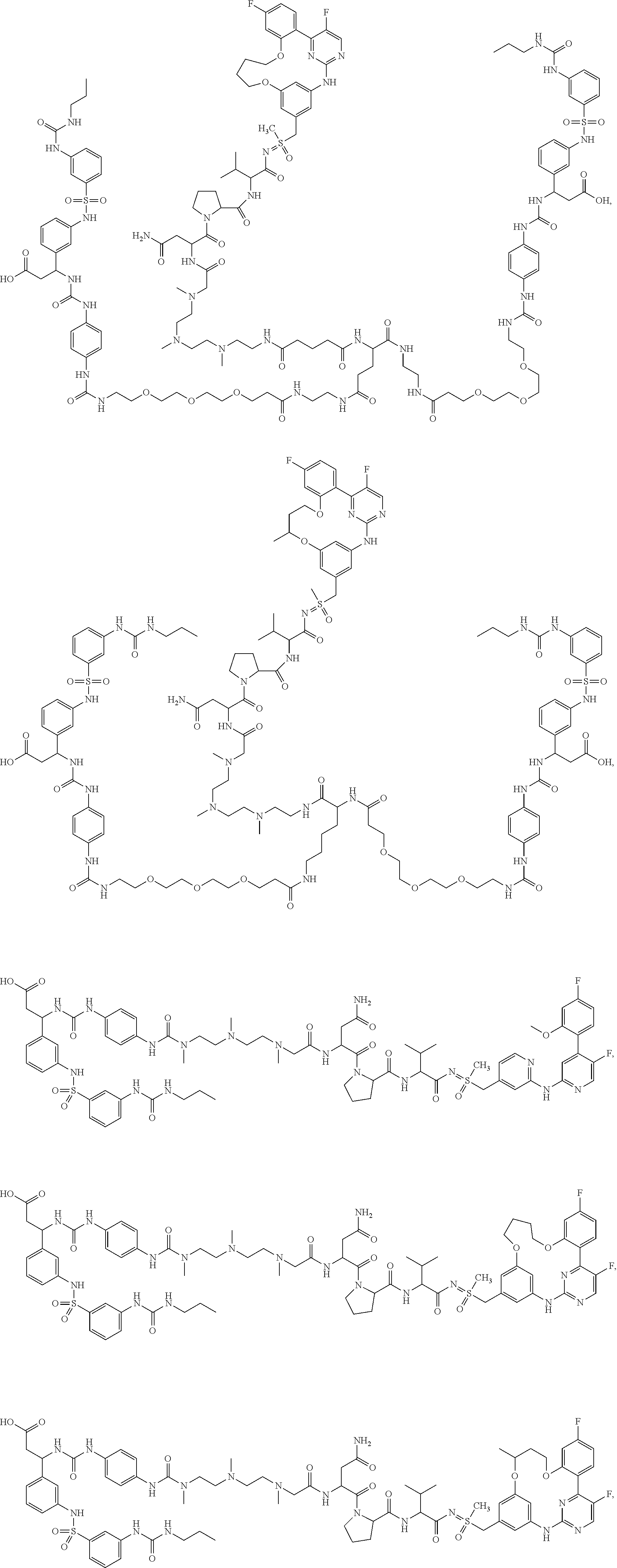 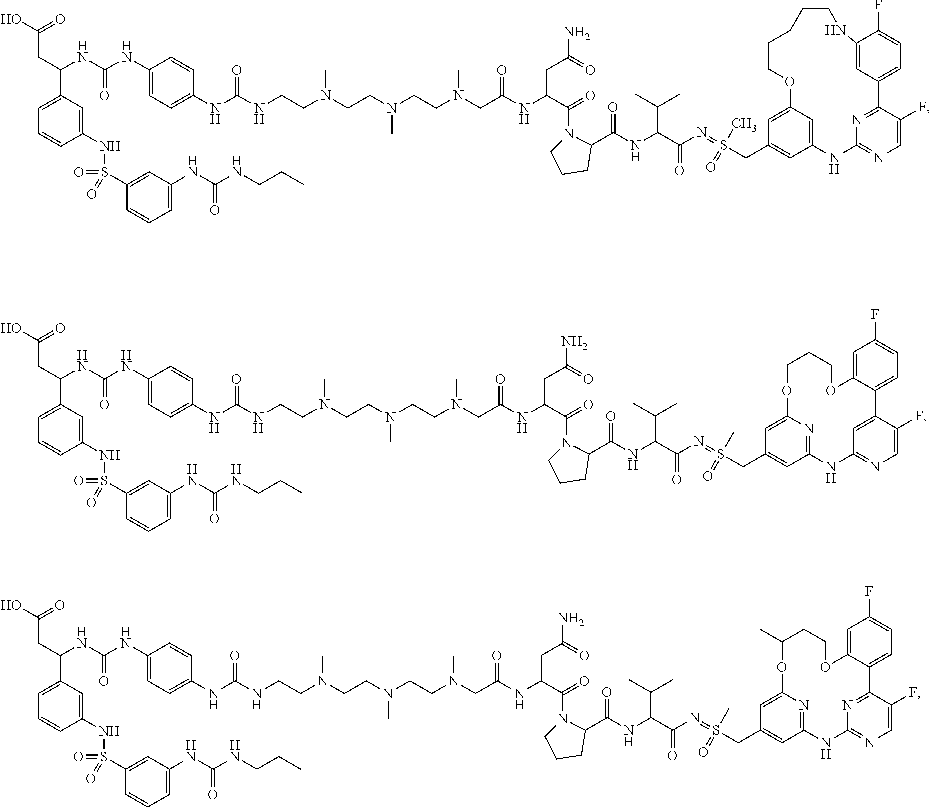
or a pharmaceutically acceptable salt thereof; or a stereoisomer or mixture of stereoisomers thereof.

37. The compound of any one of the preceding claims, or a pharmaceutically acceptable salt thereof, or a stereoisomer or mixture of stereoisomers thereof, having a structure represented by Formula (XII-C3) or Formula (XII-C4): 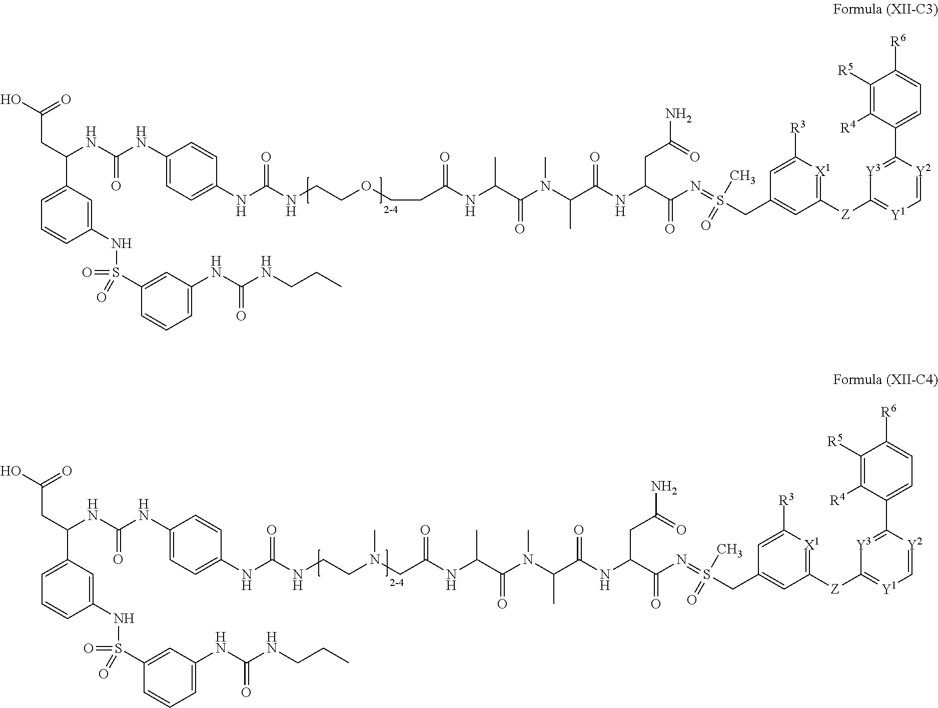
or a pharmaceutically acceptable salt thereof, or a stereoisomer or mixture of stereoisomers thereof.

38. The compound of any one of the preceding claims, or a pharmaceutically acceptable salt thereof; or a stereoisomer or mixture of stereoisomers thereof, having the structure: 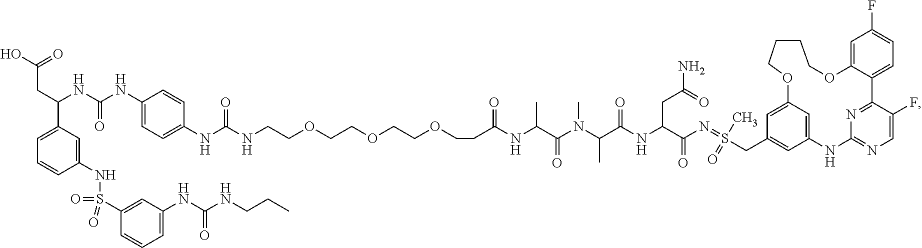 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(C)C(=O)N(C)C(C)C(=O)NC(CC(N)=O)C(=O)N=S(C)(=O)Cc4cc5cc(c4)OCCCCOc4cc(F)ccc4-c4nc(ncc4F)N5)cc3)c2)c1]
or a pharmaceutically acceptable salt thereof; or a stereoisomer or mixture of stereoisomers thereof.

39. The compound of any one of the preceding claims, or a pharmaceutically acceptable salt thereof, or a stereoisomer or mixture of stereoisomers thereof, having a structure represented by Formula (VII-C1), Formula (VII-C2), Formula (VII-C4), or Formula (VII-C5): 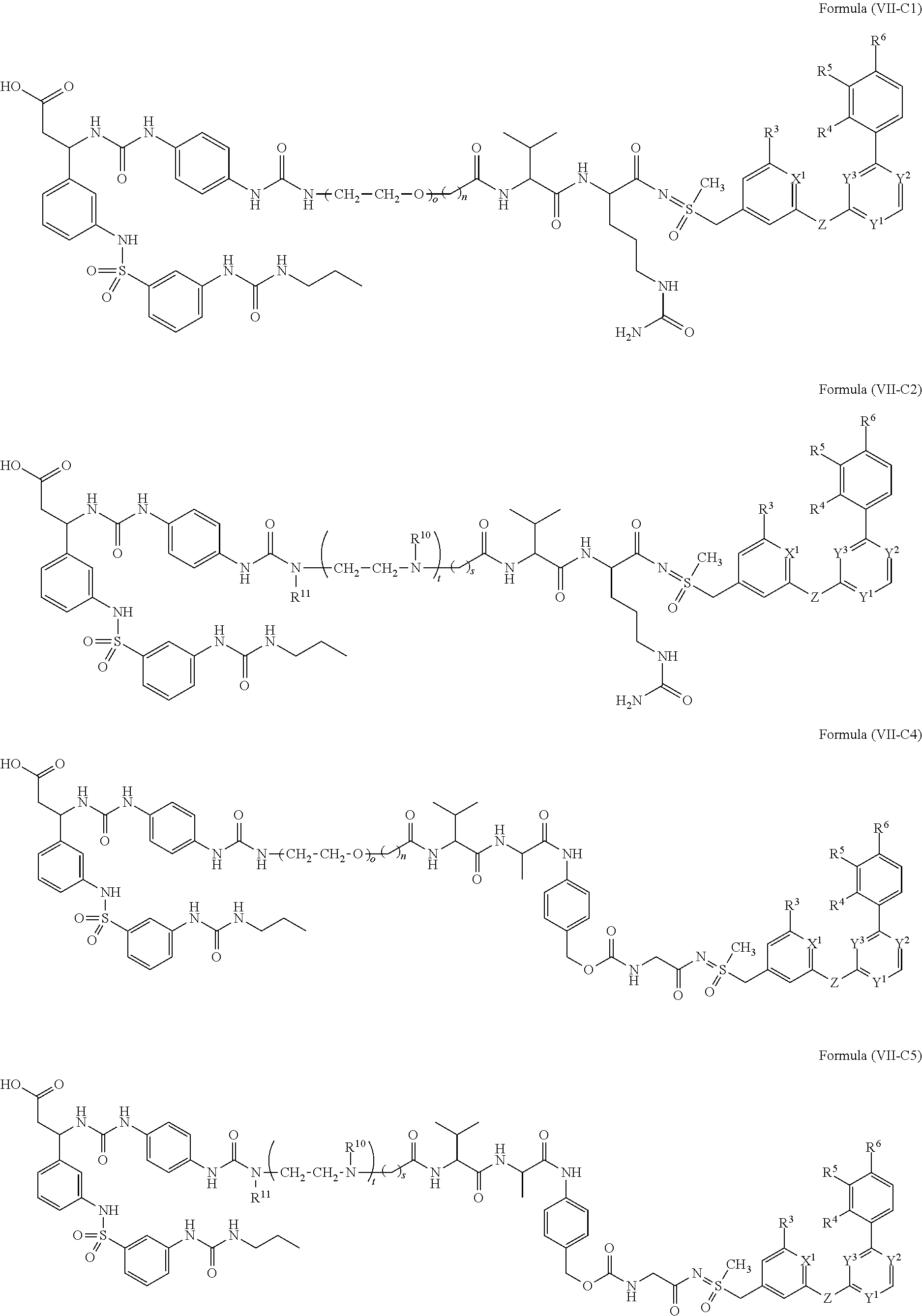
or a pharmaceutically acceptable salt thereof; or a stereoisomer or mixture of stereoisomers thereof, wherein:
X¹ is CH or N;
Y¹ is N;
Y² is CF or N;
Y³ is CH or N;
Z is —NH—;
R³ is hydrogen;
R⁴ is —OCH₃; or
R³ and R⁴ are taken together to form —O—C₂₋₁₀ alkyl-O— or —O—C₂₋₁₀ alkyl-NH—;
R⁵ is hydrogen; or
R³ and R⁵ are taken together to form —O—C₂₋₁₀ alkyl-O— or —O—C₂₋₁₀ alkyl-NH—;
R⁶ is halogen;
R¹⁰ is —CH₃;
R¹¹ is hydrogen or —CH₃;
n is 2 to 4;
o is 1 to 8;
s is 1 to 4; and
t is 1 to 8.

40. The compound of any one of the preceding claims, or a pharmaceutically acceptable salt thereof; or a stereoisomer or mixture of stereoisomers thereof, having the structure: 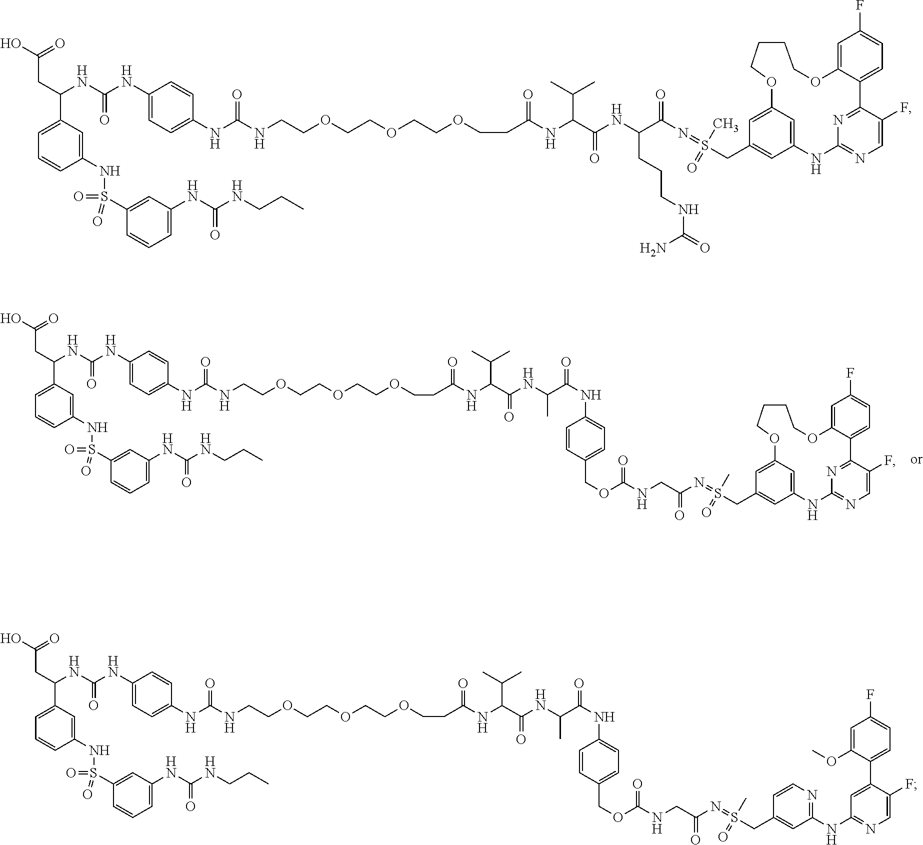
or a pharmaceutically acceptable salt thereof; or a stereoisomer or mixture of stereoisomers thereof.

41. A pharmaceutical composition comprising a compound of any one of the preceding claims, or a pharmaceutically acceptable salt thereof; or a stereoisomer or mixture of stereoisomers thereof, and at least one pharmaceutically acceptable excipient.

42. The compound of any one of the preceding claims, or a pharmaceutically acceptable salt thereof; or a stereoisomer or mixture of stereoisomers thereof; for use as a medicament.

43. The compound of any one of the preceding claims, or a pharmaceutically acceptable salt thereof; or a stereoisomer or mixture of stereoisomers thereof; for use in a method of treating a disease or disorder.

44. The compound of claim 43, or a pharmaceutically acceptable salt thereof; or a stereoisomer or mixture of stereoisomers thereof; wherein the disease or disorder is a hyperproliferative disorder.

45. The compound of claim 44, or a pharmaceutically acceptable salt thereof; or a stereoisomer or mixture of stereoisomers thereof; wherein the hyperproliferative disorder is an autoimmune disorder.

46. The compound of claim 44, or a pharmaceutically acceptable salt thereof; or a stereoisomer or mixture of stereoisomers thereof; wherein the hyperproliferative disorder is a cancer.

47. The compound of claim 46, or a pharmaceutically acceptable salt thereof; or a stereoisomer or mixture of stereoisomers thereof; wherein the cancer is a solid tumor.

48. The compound of claim 46, or a pharmaceutically acceptable salt thereof; or a stereoisomer or mixture of stereoisomers thereof; wherein the cancer is a hematological malignancy.

49. The compound of claim 46, or a pharmaceutically acceptable salt thereof; or a stereoisomer or mixture of stereoisomers thereof; wherein the cancer is a B-cell malignancy.

50. The compound of claim 46, or a pharmaceutically acceptable salt thereof; or a stereoisomer or mixture of stereoisomers thereof; wherein the cancer is a MYC-driven cancer.

51. The compound of claim 46, or a pharmaceutically acceptable salt thereof; or a stereoisomer or mixture of stereoisomers thereof; wherein the cancer is a MCL1-driven cancer.

52. The compound of claim 46, or a pharmaceutically acceptable salt thereof; or a stereoisomer or mixture of stereoisomers thereof; wherein the cancer is a tumor overexpressing MYC, MYB or MCL1 mRNA; or MYC, MCL1, or MYB proteins associated therewith.

53. The compound of claim 46, or a pharmaceutically acceptable salt thereof; or a stereoisomer or mixture of stereoisomers thereof; wherein the cancer is a transcriptionally addicted tumor.

54. The compound of claim 46, or a pharmaceutically acceptable salt thereof; or a stereoisomer or mixture of stereoisomers thereof; wherein the cancer is aggressive non-Hodgkin lymphoma (NHL), double-hit diffuse large B-cell lymphoma (DH-DLBCL), high grade B-cell lymphoma (HGBCL), transformed follicular lymphoma (FL), mantle cell lymphoma (MCL), chronic lymphocytic leukemia (CLL), small lymphocytic lymphoma (SLL), or Richter syndrome (RS).

55. The compound of claim 46, or a pharmaceutically acceptable salt thereof; or a stereoisomer or mixture of stereoisomers thereof; wherein the cancer is relapsed/refractory (r/r) aggressive non-Hodgkin lymphoma (r/r NHL), relapsed/refractory double-hit diffuse large B-cell lymphoma (r/r DH-DLBCL), relapsed/refractory high grade B-cell lymphoma (r/r HGBCL), relapsed/refractory transformed follicular lymphoma (r/r FL), relapsed/refractory mantle cell lymphoma (r/r MCL), relapsed/refractory chronic lymphocytic leukemia (r/r CLL), relapsed/refractory small lymphocytic lymphoma (r/r SLL), or relapsed/refractory Richter syndrome (r/r RS).

56. The compound of claim 46, or a pharmaceutically acceptable salt thereof; or a stereoisomer or mixture of stereoisomers thereof; wherein the cancer is ovarian cancer, breast cancer, or prostate cancer.

57. The compound of claim 46, or a pharmaceutically acceptable salt thereof; or a stereoisomer or mixture of stereoisomers thereof; wherein the cancer is advanced ovarian cancer, triple negative breast cancer, or castration-resistant neuroendocrine prostate cancer.

58. The compound of claim 46, or a pharmaceutically acceptable salt thereof; or a stereoisomer or mixture of stereoisomers thereof; wherein the cancer is neuroblastoma.

59. The compound of claim 46, or a pharmaceutically acceptable salt thereof; or a stereoisomer or mixture of stereoisomers thereof; wherein the cancer is osteosarcoma.

60. The compound of claim 43, or a pharmaceutically acceptable salt thereof; or a stereoisomer or mixture of stereoisomers thereof; wherein the disease or disorder is an ophthalmic condition.

61. The compound of claim 60, or a pharmaceutically acceptable salt, thereof, or a stereoisomer or mixture of stereoisomers thereof; wherein the ophthalmic condition is macular degeneration.

62. The compound or composition of claim 43, or a pharmaceutically acceptable salt, thereof; or a stereoisomer or mixture of stereoisomers thereof, wherein the disease or disorder is a cardiovascular condition.

63. The compound or composition of claim 62, or a pharmaceutically acceptable salt thereof, or a stereoisomer or mixture of stereoisomers thereof, wherein the cardiovascular condition is cardiac hypertrophy.

64. A method of treating a disease or disorder in a subject, comprising administering a therapeutically effective amount of a compound of any one of claims 1-40, or a pharmaceutically acceptable salt thereof; or a stereoisomer or mixture of stereoisomers thereof; or the pharmaceutical composition of claim 41, to an individual in need thereof.

65. The method of claim 64, wherein the disease or disorder is a hyperproliferative disorder.

66. The method of claim 65, wherein the hyperproliferative disorder is a cancer.

67. The method of claim 66, wherein the cancer is a MYC-, MCL1-, or MYB-driven cancer.
